(19) United States
(12) Patent Application Publication
(10) Pub. No.: US 2022/0396813 A1
(43) Pub. Date: Dec. 15, 2022

(54) RECOMBINASE COMPOSITIONS AND METHODS OF USE

(71) Applicant: Flagship Pioneering Innovations VI, LLC, Cambridge, MA (US)

(72) Inventors: Jacob Feala, Franklin, MA (US); Yanfang Fu, Arlington, MA (US); Jacob Rosenblum Rubens, Cambridge, MA (US); Robert James Citorik, Somerville, MA (US); Michael Travis Mee, Montreal (CA); Molly Krisann Gibson, Medford, MA (US)

(21) Appl. No.: 17/577,942

(22) Filed: Jan. 18, 2022

Related U.S. Application Data
(63) Continuation of application No. PCT/US2020/042511, filed on Jul. 17, 2020.
(60) Provisional application No. 63/039,328, filed on Jun. 15, 2020.
(60) Provisional application No. 62/876,165, filed on Jul. 19, 2019.

(51) Int. Cl. C12N 15/90; C12N 15/86

(52) U.S. Cl. CPC C12N 15/907; C12N 15/86; C12N 2750/14143; C12N 2800/40

(57) ABSTRACT

Methods and compositions for modulating a target genome are disclosed.

### RELATED APPLICATIONS

This application is a continuation of International Application PCT/US2020/042511, filed Jul. 17, 2020, which claims priority to U.S. Ser. No. 62/876,165 filed Jul. 19, 2019 and U.S. Ser. No. 63/039,328 filed Jun. 15, 2020, the entire contents of each of which is incorporated herein by reference.

### SEQUENCE LISTING

The instant application contains a Sequence Listing which has been submitted electronically in ASCII format and is hereby incorporated by reference in its entirety. Said ASCII copy, created on Jul. 16, 2020, is named V2065-7003WO_SL.txt and is 2,102,102 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits and are less effective at integrating longer sequences. Other existing approaches, like Cre/loxP, require a first step of inserting a loxP site into the genome and then a second step of inserting a sequence of interest into the loxP site. There is a need in the art for improved compositions (e.g., proteins and nucleic acids) and methods for inserting, altering, or deleting sequences of interest in a genome.

### SUMMARY OF THE INVENTION

This disclosure relates to novel compositions, systems and methods for altering a genome at one or more locations in a host cell, tissue or subject, in vivo or in vitro. In particular, the invention features compositions, systems and methods for the introduction of exogenous genetic elements into a host genome using a recombinase polypeptide (e.g., a tyrosine recombinase, e.g., as described herein).

### ENUMERATED EMBODIMENTS

1. A system for modifying DNA comprising:

a) a recombinase polypeptide comprising an amino acid sequence of Table 1 or 2, or an amino acid sequence having at least 70%, 75%, 80%, 85%, 90%, 95%, 96%, 97%, 98%, or 99% identity thereto, or a nucleic acid encoding the recombinase polypeptide; and

b) a double-stranded insert DNA comprising:
(i) a DNA recognition sequence that binds to the recombinase polypeptide of (a),
   said DNA recognition sequence having a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto, and
   said DNA recognition sequence further comprises a core sequence of about 5-10 nucleotides, e.g., about 8 nucleotides, wherein the core sequence is situated between the first and second parapalindromic sequences, and
(ii) a heterologous object sequence.
   2. A system for modifying DNA comprising:

a) a recombinase polypeptide comprising an amino acid sequence of Table 1 or 2, or an amino acid sequence having at least 70%, 75%, 80%, 85%, 90%, 95%, 96%, 97%, 98%, or 99% identity thereto, or a nucleic acid encoding the recombinase polypeptide; and

b) an insert DNA comprising:
(i) a human first parapalindromic sequence and a human second parapalindromic sequence of Table 1 that bind to the recombinase polypeptide of (a), and
(ii) optionally, a heterologous object sequence.
   3. The system of embodiment 1 or 2, wherein the recombinase polypeptide comprises an amino acid sequence having at least 70% sequence identity to an amino acid sequence of Table 2.
   4. The system of embodiment 1 or 2, wherein the recombinase polypeptide comprises an amino acid sequence having at least 75% sequence identity to an amino acid sequence of Table 2.
   5. The system of embodiment 1 or 2, wherein the recombinase polypeptide comprises an amino acid sequence having at least 80% sequence identity to an amino acid sequence of Table 2.
   6. The system of embodiment 1 or 2, wherein the recombinase polypeptide comprises an amino acid sequence having at least 85% sequence identity to an amino acid sequence of Table 2.
   7. The system of embodiment 1 or 2, wherein the recombinase polypeptide comprises an amino acid sequence having at least 90% sequence identity to an amino acid sequence of Table 2.
   8. The system of embodiment 1 or 2, wherein the recombinase polypeptide comprises an amino acid sequence having at least 95% sequence identity to an amino acid sequence of Table 2.
   9. The system of embodiment 1 or 2, wherein the recombinase polypeptide comprises an amino acid sequence having at least 96% sequence identity to an amino acid sequence of Table 2.
   10. The system of embodiment 1 or 2, wherein the recombinase polypeptide comprises an amino acid sequence having at least 97% sequence identity to an amino acid sequence of Table 2.
   11. The system of embodiment 1 or 2, wherein the recombinase polypeptide comprises an amino acid sequence having at least 98% sequence identity to an amino acid sequence of Table 2.
   12. The system of embodiment 1 or 2, wherein the recombinase polypeptide comprises an amino acid sequence having at least 99% sequence identity to an amino acid sequence of Table 2.
   13. The system of embodiment 1 or 2, wherein the recombinase polypeptide comprises an amino acid sequence having 100% sequence identity to an amino acid sequence of Table 2.
   14. The system of any of embodiments 1-13, wherein (a) and (b) are in separate containers.
   15. The system of any of embodiments 1-13, wherein (a) and (b) are admixed.
   16. A cell (e.g., a eukaryotic cell, e.g., a mammalian cell, e.g., human cell; or a prokaryotic cell) comprising: a recombinase polypeptide comprising an amino acid sequence of Table 1 or 2, or an amino acid sequence having at least 70%, 75%, 80%, 85%, 90%, 95%, 96%, 97%, 98%, or 99% identity thereto, or a nucleic acid encoding the recombinase polypeptide.
   17. The cell of embodiment 16, which further comprises an insert DNA comprising:

(i) a DNA recognition sequence that binds to the recombinase polypeptide, said DNA recognition sequence comprising a first parapalindromic sequence and a second parapalindromic sequence,

wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto,

wherein said DNA recognition sequence further comprises a core sequence of about 5-10 nucleotides, e.g., about 8 nucleotides, and wherein the core sequence is situated between the first and second parapalindromic sequences; and

(ii) optionally, a heterologous object sequence.

18. A cell (e.g., eukaryotic cell, e.g., mammalian cell, e.g., human cell; or a prokaryotic cell) comprising:

(i) a DNA recognition sequence, said DNA recognition sequence comprising a first parapalindromic sequence and a second parapalindromic sequence,

wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto,

wherein said DNA recognition sequence further comprises a core sequence of about 5-10 nucleotides, e.g., about 8 nucleotides, and wherein the core sequence is situated between the first and second parapalindromic sequences; and

(ii) a heterologous object sequence.

19. The cell of embodiment 18, wherein the DNA recognition sequence is within 1, 2, 3, 4, 5, 6, 7, 8, 9, 10, 15, 20, 25, 30, 40, 50, 60, 70, 80, 90, or 100 nucleotides of the heterologous object sequence.
20. The cell of embodiment 18 or 19, wherein the DNA recognition sequence and heterologous object sequence are in a chromosome or are extrachromosomal.
21. The cell of any of embodiments 16-20, wherein the cell is a eukaryotic cell.
22. The cell of embodiment 21, wherein the cell is a mammalian cell.
23. The cell of embodiment 22, wherein the cell is a human cell.
24. The cell of any of embodiments 16-20, wherein the cell is a prokaryotic cell (e.g., a bacterial cell).
25. An isolated eukaryotic cell comprising a heterologous object sequence stably integrated into its genome at a genomic location listed in column 2 or 3 of Table 1.
26. The isolated eukaryotic cell of embodiment 25, wherein the cell is an animal cell (e.g., a mammalian cell) or a plant cell.
27. The isolated eukaryotic cell of embodiment 26, wherein the mammalian cell is a human cell.
28. The isolated eukaryotic cell of embodiment 26, wherein the animal cell is a bovine cell, horse cell, pig cell, goat cell, sheep cell, chicken cell, or turkey cell.
29. The isolated eukaryotic cell of embodiment 26, wherein the plant cell is a corn cell, soy cell, wheat cell, or rice cell.
30. A method of modifying the genome of a eukaryotic cell (e.g., mammalian cell, e.g., human cell) comprising contacting the cell with:

a) a recombinase polypeptide comprising an amino acid sequence of Table 1 or 2, or a sequence having at least 70%, 75%, 80%, 85%, 90%, 95%, 96%, 97%, 98%, or 99% identity thereto, or a nucleic acid encoding the recombinase polypeptide; and

b) an insert DNA comprising:
(i) a DNA recognition sequence that binds to the recombinase polypeptide of (a), said DNA recognition sequence comprising a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto,
wherein said DNA recognition sequence further comprises a core sequence of about 5-10 nucleotides, e.g., about 8 nucleotides, and wherein the core sequence is situated between the first and second parapalindromic sequences, and
(ii) a heterologous object sequence, thereby modifying the genome of the eukaryotic cell.
   31. A method of inserting a heterologous object sequence into the genome of a eukaryotic cell (e.g., mammalian cell, e.g., human cell) comprising contacting the cell with:

a) a recombinase polypeptide comprising an amino acid sequence of Table 1 or 2, or a sequence having at least 70%, 75%, 80%, 85%, 90%, 95%, 96%, 97%, 98%, or 99% identity thereto, or a nucleic acid encoding the polypeptide; and

b) an insert DNA comprising:
(i) a DNA recognition sequence that binds to the recombinase polypeptide of (a), said DNA recognition sequence comprising a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1 or 2, and
wherein said DNA recognition sequence further comprises a core sequence of about 5-10 nucleotides, e.g., about 8 nucleotides, and wherein the core sequence is situated between the first and second parapalindromic sequences, and
(ii) a heterologous object sequence,
thereby inserting the heterologous object sequence into the genome of the eukaryotic cell, e.g., at a frequency of at least about 0.1% (e.g., at least about 0.1%, 0.5%, 1%, 5%, 10%, 20%, 30%, 40%, 50%, 60%, 70%, 80%, 85%, 90%, 95%, 96%, 97%, 98%, 99%, or 100% of a population of the eukaryotic cell, e.g., as measured in an assay of Example 5.
   32. The method of embodiment 30 or 31, wherein (a) and (b) are administered separately or together.
   33. The method of embodiment 30 or 31, wherein (a) is administered prior to, concurrently with, or after administration of (b).
   34. The method of any of embodiments 30-33, wherein (a) comprises the nucleic acid encoding the polypeptide.
   35. The method of embodiment 34, wherein the nucleic acid of (a) and the insert DNA of (b) are situated on the same nucleic acid molecule, e.g., are situated on the same vector.
   36. The method of embodiment 34, wherein the nucleic acid of (a) and the insert DNA of (b) are situated on separate nucleic acid molecules.
   37. The method of any of embodiments 30-36, wherein the cell has only one endogenous DNA recognition sequence that is compatible with the DNA recognition sequence of the insert DNA.
   38. The method of any of embodiments 30-36, wherein the cell has two or more endogenous DNA recognition sequences that are compatible with the DNA recognition sequence of the insert DNA.
   39. An isolated recombinase polypeptide comprising an amino acid sequence of Table 1 or 2, or a sequence having at least 70%, 75%, 80%, 85%, 90%, 95%, 96%, 97%, 98%, or 99% identity thereto.
   40. The isolated recombinase polypeptide of embodiment 39, which comprises at least one insertion, deletion, or substitution relative to a recombinase sequence of Table 1 or 2.
   41. The isolated recombinase polypeptide of embodiment 40, wherein the synthetic recombinase polypeptide binds a eukaryotic (e.g., mammalian, e.g., human) genomic locus (e.g., a sequence of Table 1).
   42. The isolated recombinase polypeptide of embodiment 40 or 41, wherein the synthetic recombinase polypeptide has at least a 2-, 3-, 4-, or 5-fold increase in affinity for the genomic locus, relative to the corresponding unmodified amino acid sequence of Table 1 or 2.
   43. An isolated nucleic acid encoding a recombinase polypeptide comprising an amino acid sequence of Table 1 or 2, or an amino acid sequence having at least 70%, 75%, 80%, 85%, 90%, 95%, 96%, 97%, 98%, or 99% identity thereto.
   44. The isolated nucleic acid of embodiment 43, which encodes a recombinase polypeptide comprising at least one insertion, deletion, or substitution relative to a recombinase sequence of Table 1 or 2.
   45. The isolated nucleic acid sequence of embodiment 43 or 44, which is codon-optimized for mammalian cells, e.g., human cells.
   46. The isolated nucleic acid of any of embodiments 43-45, which further comprises a heterologous promoter (e.g., a mammalian promoter, e.g., a tissue-specific promoter), microRNA (e.g., a tissue-specific restrictive miRNA), polyadenylation signal, or a heterologous payload.
   47. An isolated nucleic acid (e.g., DNA) comprising:

(i) a DNA recognition sequence, said DNA recognition sequence comprising a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, and

said DNA recognition sequence further comprises a core sequence of about 5-10 nucleotides, e.g., about 8 nucleotides, wherein the core sequence is situated between the first and second parapalindromic sequences, and

(ii) a heterologous object sequence.

48. The isolated nucleic acid of embodiment 47, which binds to a recombinase polypeptide of Table 1 or 2.
49. A method of making a recombinase polypeptide, the method comprising:

a) providing a nucleic acid encoding a recombinase polypeptide comprising an amino acid sequence of Table 1 or 2, or a sequence having at least 70%, 75%, 80%, 85%, 90%, 95%, 96%, 97%, 98%, or 99% identity thereto, and

b) introducing the nucleic acid into a cell (e.g., a eukaryotic cell or a prokaryotic cell, e.g., as described herein) under conditions that allow for production of the recombinase polypeptide,

thereby making the recombinase polypeptide.

50. A method of making a recombinase polypeptide, the method comprising:

a) providing a cell (e.g., a prokaryotic or eukaryotic cell) comprising a nucleic acid encoding a recombinase polypeptide comprising an amino acid sequence of Table 1 or 2, or a sequence having at least 70%, 75%, 80%, 85%, 90%, 95%, 96%, 97%, 98%, or 99% identity thereto, and

b) incubating the cell under conditions that allow for production of the recombinase polypeptide,

thereby making the recombinase polypeptide.

51. A method of making an insert DNA that comprises a DNA recognition sequence and a heterologous sequence, comprising:

a) providing a nucleic acid comprising:
(i) a DNA recognition sequence that binds to a recombinase polypeptide comprising an amino acid sequence of Table 1 or 2, or a sequence having at least 70%, 75%, 80%, 85%, 90%, 95%, 96%, 97%, 98%, or 99% identity thereto, said DNA recognition sequence comprising a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1, and
said DNA recognition sequence further comprises a core sequence of about 5-10 nucleotides, e.g., about 8 nucleotides, wherein the core sequence is situated between the first and second parapalindromic sequences, and
(ii) a heterologous object sequence, and

b) introducing the nucleic acid into a cell (e.g., a eukaryotic cell or a prokaryotic cell, e.g., as described herein) under conditions that allow for replication of the nucleic acid,

thereby making the insert DNA.

52. The system, cell, method, isolated recombinase polypeptide, or isolated nucleic acid of any of the preceding embodiments, wherein the recombinase polypeptide comprises at least one insertion, deletion, or substitution relative to the amino acid sequence of Table 1 or 2.
53. The system, cell, method, isolated recombinase polypeptide, or isolated nucleic acid of any of the preceding embodiments, wherein the recombinase polypeptide comprises a truncation at the N-terminus, C-terminus, or both of the N- and C-termini relative to the amino acid sequence of Table 1 or 2.
54. The system, cell, method, isolated recombinase polypeptide, or isolated nucleic acid of any of the preceding embodiments, wherein the recombinase polypeptide comprises a nuclear localization sequence, e.g., an endogenous nuclear localization sequence or a heterologous nuclear localization sequence.
55. The system, cell, method, isolated recombinase polypeptide, or isolated nucleic acid of any of the preceding embodiments, wherein the heterologous object sequence is inserted into the genome of the cell at an efficiency of at least about 0.1% (e.g., at least about 0.1%, 0.5%, 1%, 5%, 10%, 20%, 30%, 40%, 50%, 60%, 70%, 80%, 85%, 90%, 95%, 96%, 97%, 98%, 99%, or 100%) of a population of the cell, e.g., as measured in an assay of Example 5.
56. The system, cell, method, isolated recombinase polypeptide, or isolated nucleic acid of any of the preceding embodiments, wherein the heterologous object sequence is inserted into a site within the genome of the cell (e.g., a locus listed in column 4 of Table 1, e.g., corresponding to the row for a recombinase listed in column 1 of Table 1) in at least about 1%, (e.g., at least about 1%, 5%, 10%, 15%, 20%, 25%, 30%, 40%, 50%, 60%, 70%, 80%, 85%, 90%, 95%, 96%, 97%, 98%, 99%, 99.9%, or 100%) of insertion events, e.g., as measured by an assay of Example 4.
57. The system, cell, method, isolated recombinase polypeptide, or isolated nucleic acid of any of the preceding embodiments, wherein, in a population of the cells (e.g., contacted with the system), the heterologous object sequence is inserted into between 1-10, e.g., 1-9, 1-8, 1-7, 1-6, 1-5, 1-4, 1-3, 2-10, 2-5, 2-4, 3-10, 3-5, or 5-10 sites within the genome of the cell (e.g., a locus listed in column 4 of Table 1, e.g., corresponding to the row for a recombinase listed in column 1 of Table 1), in at least 1%, 5%, 10%, 15%, 20%, 25%, 30%, 40%, 50%, 60%, 70%, 80%, 85%, 90%, 95%, 96%, 97%, 98%, 99%, 99.9%, or 100%) of the cells in the population, e.g., as measured by an assay of Example 4.
58. The system, cell, method, isolated recombinase polypeptide, or isolated nucleic acid of any of the preceding embodiments, wherein, in a population of cells contacted with the system, the heterologous object sequence is inserted into exactly one site within the genome of the cell (e.g., a locus listed in column 4 of Table 1, e.g., corresponding to the row for a recombinase listed in column 1 of Table 1), in at least 1%, 5%, 10%, 15%, 20%, 25%, 30%, 40%, 50%, 60%, 70%, 80%, 85%, 90%, 95%, 96%, 97%, 98%, 99%, 99.9%, or 100%) of the cells in the population, e.g., as measured by an assay of Example 4.
59. The system, cell, method, isolated recombinase polypeptide, or isolated nucleic acid of any of the preceding embodiments, wherein the heterologous object sequence is inserted into between 1-10, e.g., 1-9, 1-8, 1-7, 1-6, 1-5, 1-4, 1-3, 2-10, 2-5, 2-4, 3-10, 3-5, or 5-10 sites within the genome of the cell (e.g., a locus listed in column 4 of Table 1, e.g., corresponding to the row for a recombinase listed in column 1 of Table 1), e.g., as measured by an assay of Example 4.
60. The system, cell, method, isolated recombinase polypeptide, or isolated nucleic acid of any of the preceding embodiments, wherein the recombinase polypeptide is bound to the insert DNA.
61. The system, cell, method, isolated recombinase polypeptide, or isolated nucleic acid of any of the preceding embodiments, wherein the recombinase polypeptide is provided by providing a nucleic acid encoding the recombinase polypeptide.
62. The system, cell, method, isolated recombinase polypeptide, or isolated nucleic acid of any of the preceding embodiments, which results in an insert frequency of the heterologous object sequence into the genome of at least about 0.1% (e.g., at least about 0.1%, 0.5%, 1%, 5%, 10%, 20%, 30%, 40%, 50%, 60%, 70%, 80%, 85%, 90%, 95%, 96%, 97%, 98%, 99%, or 100%) of a population of the cells, e.g., as measured in an assay of Example 5.
63. The system, cell, method, isolated recombinase polypeptide, or isolated nucleic acid of any of the preceding embodiments, wherein the first parapalindromic sequence comprises a sequence comprising the first 10-30, 12-27, or 10-15, e.g., 10, 11, 12, 13, 14, or 15 nucleotides of the nucleotide sequence of column 2 or column 3 of Table 1, or a sequence having no more than 1, 2, or 3 substitutions, insertions, or deletions relative thereto.
64. The system, cell, method, isolated recombinase polypeptide, or isolated nucleic acid of embodiment 63, wherein the second parapalindromic sequence further comprises a second sequence comprising the last 10-30, 12-27, or 10-15, e.g., 10, 11, 12, 13, 14, or 15 nucleotides of the same nucleotide sequence of column 2 or column 3 of Table 1, or a sequence having no more than 1, 2, or 3 substitutions, insertions, or deletions relative thereto.
65. The system, cell, method, isolated recombinase polypeptide, or isolated nucleic acid of any of the preceding embodiments, wherein the insert DNA further comprises a core sequence comprising the 8 nucleotides situated between the parapalindromic regions of column 3 of Table 1, or a sequence having no more than 1, 2, or 3 substitutions, insertions, or deletions relative thereto.
66. The system, cell, method, isolated recombinase polypeptide, or isolated nucleic acid of any of the preceding embodiments, wherein the first and second parapalindromic sequences comprise a perfectly palindromic sequence.
67. The system, cell, method, isolated recombinase polypeptide, or isolated nucleic acid of any of the preceding embodiments, wherein the parapalindromic sequence comprises 1, 2, 3, 4, 5, or 6 non-palindromic positions.
68. The system, cell, method, isolated recombinase polypeptide, or isolated nucleic acid of any of the preceding embodiments, wherein the parapalindromic region comprises a 5′ region of 10-30, 12-27, or 10-15, e.g., about 13 nucleotides and/or a 3′ region of 10-30, 12-27, or 10-15, e.g., about 13 nucleotides.
69. The system, cell, method, isolated recombinase polypeptide, or isolated nucleic acid of any of the preceding embodiments, wherein the first and second parapalindromic sequences are the same length.
70. The system, cell, method, isolated recombinase polypeptide, or isolated nucleic acid of any of the preceding embodiments, wherein the core sequence is 5-10 nucleotides (e.g., about 8 nucleotides) in length.
71. The system, cell, method, isolated recombinase polypeptide, or isolated nucleic acid of any of the preceding embodiments, wherein the core sequence is capable of hybridizing to a corresponding sequence in the human genome, or the reverse complement thereof.
72. The system, cell, method, isolated recombinase polypeptide, or isolated nucleic acid of any of the preceding embodiments, wherein the core sequence has at least 10%, 20%, 30%, 40%, 50%, 60%, 70%, 80%, or 90% identity to a corresponding sequence in the human genome.
73. The system, cell, method, isolated recombinase polypeptide, or isolated nucleic acid of any of the preceding embodiments, wherein the core sequence has no more than 1, 2, 3, 4, 5, 6, 7, 8, or 9 mismatches to a corresponding sequence in the human genome.
74. The system, cell, method, isolated recombinase polypeptide, or isolated nucleic acid of any of the preceding embodiments, wherein the core sequence, when cleaved by the recombinase, forms a sticky end that is capable of hybridizing to a corresponding sequence in the human genome.
75. The system, cell, method, isolated recombinase polypeptide, or isolated nucleic acid of any of the preceding embodiments, wherein the heterologous object sequence comprises a eukaryotic gene, e.g., a mammalian gene, e.g., human gene, e.g., a blood factor (e.g., genome factor I, II, V, VII, X, XI, XII or XIII) or enzyme, e.g., lysosomal enzyme, or synthetic human gene (e.g. a chimeric antigen receptor).
76. The system, cell, method, isolated recombinase polypeptide, or isolated nucleic acid of any of the preceding embodiments, wherein the insert DNA comprises a heterologous object sequence and a DNA recognition sequence.
77. The system, cell, method, isolated recombinase polypeptide, or isolated nucleic acid of any of the preceding embodiments, wherein the insert DNA comprises a nucleic acid sequence encoding the recombinase polypeptide.
78. The system, cell, method, isolated recombinase polypeptide, or isolated nucleic acid of any of the preceding embodiments, wherein the insert DNA and a nucleic acid encoding the recombinase polypeptide are present in separate nucleic acid molecules.
79. The system, cell, method, isolated recombinase polypeptide, or isolated nucleic acid of any of embodiments 1-77, wherein the insert DNA and a nucleic acid encoding the recombinase polypeptide are present in the same nucleic acid molecule.
80. The system, cell, method, isolated recombinase polypeptide, or isolated nucleic acid of any of the preceding embodiments, wherein the insert DNA further comprises 1, 2, 3, 4, 5, or all of:
(a) an open reading frame, e.g., a sequence encoding a polypeptide, e.g., an enzyme (e.g., a lysosomal enzyme), a blood factor, an exon.
(b) a non-coding and/or regulatory sequence, e.g., a sequence that binds a transcriptional modulator, e.g., a promoter (e.g., a heterologous promoter), an enhancer, an insulator.
(c) a splice acceptor site;
(d) a polyA site;
(e) an epigenetic modification site; or
(f) a gene expression unit.
   81. The system, cell, method, isolated recombinase polypeptide, or isolated nucleic acid of any of the preceding embodiments, wherein the insert DNA comprises a plasmid, viral vector (e.g., lentiviral vector or episomal viral vector), or other self-replicating vector.
   82. The system, cell, method, isolated recombinase polypeptide, or isolated nucleic acid of any of the preceding embodiments, wherein the cell does not comprise an endogenous human gene comprised by the heterologous object sequence, or does not comprise a protein encoded by said gene.
   83. The system, cell, method, isolated recombinase polypeptide, or isolated nucleic acid of any of the preceding embodiments, wherein the cell is from an organism that does not comprise an endogenous human gene comprised by the heterologous object sequence, or does not comprise a protein encoded by said gene.
   84. The system, cell, method, isolated recombinase polypeptide, or isolated nucleic acid of any of the preceding embodiments, wherein the cell comprises an endogenous human DNA recognition sequence.
   85. The system, cell, method, isolated recombinase polypeptide, or isolated nucleic acid of embodiment 84, wherein the endogenous human DNA recognition sequence is operably linked to, e.g., is situated in a site within the human genome having at least 1, 2, 3, 4, 5, 6, 7, 8 or 9 of the following criteria:
   (i) is located >300 kb from a cancer-related gene;
   (ii) is >300 kb from a miRNA/other functional small RNA;
   (iii) is >50 kb from a 5′ gene end;
   (iv) is >50 kb from a replication origin;
   (v) is >50 kb away from any ultraconserved element;
   (vi) has low transcriptional activity (i.e. no mRNA+/−25 kb); (vii) is not in copy number variable region;
   (viii) is in open chromatin; and/or
   (ix) is unique, e.g., with 1 copy in the human genome.
   86. The system, cell, method, isolated recombinase polypeptide, or isolated nucleic acid of any of the preceding embodiments, wherein the cell is an animal cell, e.g., a mammalian cell, e.g., a human cell.
   87. The system, cell, method, isolated recombinase polypeptide, or isolated nucleic acid of any of the preceding embodiments, wherein the cell is a plant cell.
   88. The system, cell, method, isolated recombinase polypeptide, or isolated nucleic acid of any of the preceding embodiments, wherein the cell is not genetically modified.
   89. The system, cell, method, isolated recombinase polypeptide, or isolated nucleic acid of any of the preceding embodiments, wherein the cell does not comprise a loxP site.
   90. The system or method of any of the preceding embodiments, wherein the nucleic acid encoding the recombinase polypeptide is in a viral vector, e.g., an AAV vector.
   91. The system or method of any of the preceding embodiments, wherein the double-stranded insert DNA is in a viral vector, e.g., an AAV vector.
   92. The system or method of any of the preceding embodiments, wherein the nucleic acid encoding the recombinase polypeptide is an mRNA, wherein optionally the mRNA is in an LNP.
   93. The system or method of any of the preceding embodiments, wherein the double-stranded insert DNA is not in a viral vector, e.g., wherein the double-stranded insert DNA is naked DNA or DNA in a transfection reagent.
   94. The system or method of any of the preceding embodiments, wherein:

the nucleic acid encoding the recombinase polypeptide is in a first viral vector, e.g., a first AAV vector, and

the insert DNA is in a second viral vector, e.g., a second AAV vector.

95. The system or method of any of the preceding embodiments, wherein:

the nucleic acid encoding the recombinase polypeptide is an mRNA, wherein optionally the mRNA is in an LNP, and

the insert DNA is in a viral vector, e.g., an AAV vector.

96. The system or method of any of the preceding embodiments, wherein:

the nucleic acid encoding the recombinase polypeptide is an mRNA, and

the double-stranded insert DNA is not in a viral vector, e.g., wherein the double-stranded insert DNA is naked DNA or DNA in a transfection reagent.

97. The system or method of any of the preceding embodiments, wherein the insert DNA has a length of at least 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, 130 kb, 140 kb, or 150 kb.
98. The system or method of any of the preceding embodiments, wherein the insert DNA does not comprise an antibiotic resistance gene or any other bacterial genes or parts.
99. The system, cell, polypeptide, nucleic acid, or method of any of the preceding embodiments, wherein the recombinase polypeptide is a recombinase selected from Rec17 (SEQ ID NO: 1231), Rec19 (SEQ ID NO: 1233), Rec20 (SEQ ID NO: 1234), Rec27 (SEQ ID NO: 1241), Rec29 (SEQ ID NO: 1243), Rec30 (SEQ ID NO: 1244), Rec31 (SEQ ID NO: 1245), Rec32 (SEQ ID NO: 1246), Rec33 (SEQ ID NO: 1247), Rec34 (SEQ ID NO: 1248), Rec35 (SEQ ID NO: 1249), Rec36 (SEQ ID NO: 1250), Rec37 (SEQ ID NO: 1251), Rec38 (SEQ ID NO: 1252), Rec39 (SEQ ID NO: 1253), Rec338 (SEQ ID NO: 1552), or Rec589 (SEQ ID NO: 1803), or a recombinase polypeptide having an amino acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 15, 20, 25, 30, 35, 40, 45, or 50 sequence alterations (e.g., substitutions, insertions, or deletions) relative thereto.
100. The system, cell, polypeptide, nucleic acid, or method of any of the preceding embodiments, wherein when the polypeptide, system, or nucleic acid is used in a reporter gene inversion assay, e.g., an assay of Example 13, it results in reporter gene expression in at least 1, 2, 3, 4, 5, 6, 7, 8, 9, 10, 11, 12, 13, 14, 15, 20, 25, 30, 35, 40, 45, 50, 55, or 60% of cells.
101. The system, cell, polypeptide, nucleic acid, or method of any of the preceding embodiments, wherein the reporter gene inversion assay comprises:

i) introducing the polypeptide, system, or nucleic acid into a test population of cells,

ii) introducing into the test population of cells a nucleic acid comprising from 5′ to 3′ a promoter, a first DNA recognition sequence that binds the recombinase polypeptide, a GFP gene in antisense orientation, and a second DNA recognition sequence that binds the recombinase polypeptide (e.g., wherein the first and second DNA recognition sequences each comprise one or more sequences from column 3 of Table 1 from the same row as the corresponding recombinase polypeptide),

iii) incubating the test population of cells for a time sufficient to allow for inversion of the GFP gene, e.g., for 2 days at 37° C., e.g., as described in Example 13, and

iv) determining a value for the percentage of cells in the test population that display GFP fluorescence, e.g., wherein the threshold for GFP fluorescence is at least 1.7× (1.7 times), 1.8×, 1.9×, 2×, 2.1×, 2.2×, or 2.3× (e.g., 2×) the background fluorescence, e.g., as described in Example 13.

102. The system, cell, polypeptide, nucleic acid, or method of any of the preceding embodiments, wherein when the polypeptide, system, or nucleic acid is used in a reporter gene integration assay, e.g., an assay of Example 14, it results in an average reporter gene copy number of at least 0.05, 0.1, 0.15, 0.2, 0.25, 0.3, 0.35, 0.4, 0.45, 0.5, 0.55, 0.6, 0.7, 0.8, 0.9, or 0.95 per cell.
103. The system, cell, polypeptide, nucleic acid, or method of any of the preceding embodiments, wherein the reporter gene integration assay comprises:

i) introducing the polypeptide, system, or nucleic acid into a test population of cells,

ii) introducing into the test population of cells a nucleic acid comprising from 5′ to 3′ a first DNA recognition sequence that binds the recombinase polypeptide, a GFP gene, and a second DNA recognition sequence that binds the recombinase polypeptide (e.g., wherein the first and second DNA recognition sequences each comprise one or more sequences from column 3 of Table 1 from the same row as the corresponding recombinase polypeptide),

iii) incubating the test population of cells for a time sufficient to allow for integration of the GFP gene into the genomic DNA of the test population of cells, e.g., for 2-5 days at 37° C., e.g., as described in Example 14, and

iv) determining a value for the average copy number of GFP gene per cell in the genomic DNA of the test population of cells, e.g., wherein the threshold copy number is at least 1.7× (1.7 times), 1.8×, 1.9×, 2×, 2.1×, 2.2×, or 2.3× (e.g., 2×) the background copy number detected, e.g., as described in Example 14.

104. The system, cell, polypeptide, nucleic acid, or method of any of the preceding embodiments, wherein the nucleic acid (e.g., isolated nucleic acid), insert DNA (e.g., double-stranded insert DNA), or heterologous object sequence comprises an artificial chromosome, e.g., a bacterial artificial chromosome.
105. The system, cell, polypeptide, or nucleic acid of any of the preceding embodiments for use as a laboratory or research tool, or in a laboratory method or research method.
106. The method of any of embodiments 30-38 or 52-104, wherein the method is used as a laboratory or research method or as part of a laboratory or research method.
107. The system, cell, polypeptide, nucleic acid, or method of either of embodiments 105 or 106, wherein the laboratory or research tool or laboratory or research method is used to modify an animal cell, e.g., a mammalian cell (e.g., a human cell), a plant cell, or a fungal cell.
108. The system, cell, polypeptide, nucleic acid, or method of any of embodiments 105-107, wherein the laboratory or research tool or laboratory or research method is used in vitro.

The disclosure contemplates all combinations of any one or more of the foregoing aspects and/or embodiments, as well as combinations with any one or more of the embodiments set forth in the detailed description and examples.

### Definitions

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, a nuclear localization sequence, a recombinase domain, a DNA recognition domain (e.g., that binds to or is capable of binding to a recognition site, e.g. as described herein), a tyrosine recombinase N-terminal domain, and a tyrosine recombinase C-terminal domain; an example of a domain of a nucleic acid is a regulatory domain, such as a transcription factor binding domain, a parapalindromic sequence, a parapalindromic region, a core sequence, or an object sequence (e.g., a heterologous object sequence). In some embodiments, a recombinase polypeptide comprises one or more domains (e.g., a recombinase domain, or a DNA recognition domain) of a polypeptide of Table 1 or 2, or a fragment or variant thereof.

Exogenous: As used herein, the term exogenous, when used with reference to a biomolecule (such as a nucleic acid sequence or polypeptide) means that the biomolecule was introduced into a host genome, cell or organism by the hand of man. For example, a nucleic acid that is as added into an existing genome, cell, tissue or subject using recombinant DNA techniques or other methods is exogenous to the existing nucleic acid sequence, cell, tissue or subject.

Genomic safe harbor site (GSH site): A genomic safe harbor site is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA+/−25 kb); (vii) is not in a copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C-C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

Heterologous: The term heterologous, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art.

Nucleic acid molecule: Nucleic acid molecule refers to both RNA and DNA molecules including, without limitation, cDNA, genomic DNA and mRNA, and also includes synthetic nucleic acid molecules, such as those that are chemically synthesized or recombinantly produced, such as DNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ ID NO:,” “nucleic acid comprising SEQ ID NO:1” refers to a nucleic acid, at least a portion which has either (i) the sequence of SEQ ID NO:1, or (ii) a sequence complimentary to SEQ ID NO:1. The choice between the two is dictated by the context in which SEQ ID NO:1 is used. For instance, if the nucleic acid is used as a probe, the choice between the two is dictated by the requirement that the probe be complimentary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids.

Gene expression unit: a gene expression unit is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

Host: The terms host genome or host cell, as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a bovine cell, horse cell, pig cell, goat cell, sheep cell, chicken cell, or turkey cell. In certain instances, a host cell may be a corn cell, soy cell, wheat cell, or rice cell.

Recombinase polypeptide: As used herein, a recombinase polypeptide refers to a polypeptide having the functional capacity to catalyze a recombination reaction of a nucleic acid molecule (e.g., a DNA molecule). A recombination reaction may include, for example, one or more nucleic acid strand breaks (e.g., a double-strand break), followed by joining of two nucleic acid strand ends (e.g., sticky ends). In some instances, the recombination reaction comprises insertion of an insert nucleic acid, e.g., into a target site, e.g., in a genome or a construct. In some instances, a recombinase polypeptide comprises one or more structural elements of a naturally occurring recombinase (e.g., a tyrosine recombinase, e.g., Cre recombinase or Flp recombinase). In certain instances, a recombinase polypeptide comprises an amino acid sequence having at least 70%, 75%, 80%, 85%, 90%, 95%, 96%, 97%, 98%, 99%, or 100% sequence identity to a recombinase described herein (e.g., as listed in Table 1 or 2). In some instances, a recombinase polypeptide has one or more functional features of a naturally occurring recombinase (e.g., a tyrosine recombinase, e.g., Cre recombinase or Flp recombinase). In some instances, a recombinase polypeptide recognizes (e.g., binds to) a recognition sequence in a nucleic acid molecule (e.g., a recognition sequence listed in Table 1 or 2, or a sequence having at least 70%, 75%, 80%, 85%, 90%, 95%, 96%, 97%, 98%, or 99% identity thereto). In some embodiments, a recombinase polypeptide is not active as an isolated monomer. In some embodiments, a recombinase polypeptide catalyzes a recombination reaction in concert with one or more other recombinase polypeptides (e.g., four recombinase polypeptides per recombination reaction).

Insert nucleic acid molecule: As used herein, an insert nucleic acid molecule (e.g., an insert DNA) is a nucleic acid molecule (e.g., a DNA molecule) that is or will be inserted, at least partially, into a target site within a target nucleic acid molecule (e.g., genomic DNA). An insert nucleic acid molecule may include, for example, a nucleic acid sequence that is heterologous relative to the target nucleic acid molecule (e.g., the genomic DNA). In some instances, an insert nucleic acid molecule comprises an object sequence (e.g., a heterologous object sequence). In some instances, an insert nucleic acid molecule comprises a DNA recognition sequence, e.g., a cognate to a DNA recognition sequence present in a target nucleic acid. In some embodiments, the insert nucleic acid molecule is circular, and in some embodiments, the insert nucleic acid molecule is linear. In some embodiments, an insert nucleic acid molecule is also referred to as a template nucleic acid molecule (e.g., a template DNA).

Recognition sequence: A recognition sequence (e.g., DNA recognition sequence) generally refers to a nucleic acid (e.g., DNA) sequence that is recognized (e.g., capable of being bound by) a recombinase polypeptide, e.g., as described herein. In some instances, a recognition sequence comprises two parapalindromic sequences, e.g., as described herein. In certain instances, the two parapalindromic sequences together form a parapalindromic region or a portion thereof. In some instances, the recognition sequence further comprises a core sequence, e.g., as described herein, positioned between the two parapalindromic sequences. In some instances, a recognition sequence comprises a nucleic acid sequence listed in Table 1, or a sequence having at least 70%, 75%, 80%, 85%, 90%, 95%, 96%, 97%, 98%, or 99% identity thereto.

Core sequence: A core sequence, as used herein, refers to a nucleic acid sequence positioned between two parapalindromic sequences. In some instances, a core sequence can be cleaved by a recombinase polypeptide (e.g., a recombinase polypeptide that recognizes a recognition sequence comprising the two parapalindromic sequences), e.g., to form sticky ends. In some embodiments, the core sequence is about 5-10 nucleotides, e.g., about 8 nucleotides in length.

Object sequence: As used herein, the term object sequence refers to a nucleic acid segment that can be desirably inserted into a target nucleic acid molecule, e.g., by a recombinase polypeptide, e.g., as described herein. In some embodiments, an insert DNA comprises a DNA recognition sequence and an object sequence that is heterologous to the DNA recognition sequence, generally referred to herein as a “heterologous object sequence.” An object sequence may, in some instances, be heterologous relative to the nucleic acid molecule into which it is inserted. In some instances, an object sequence comprises a nucleic acid sequence encoding a gene (e.g., a eukaryotic gene, e.g., a mammalian gene, e.g., a human gene) or other cargo of interest (e.g., a sequence encoding a functional RNA, e.g., an siRNA or miRNA), e.g., as described herein. In certain instances, the gene encodes a polypeptide (e.g., a blood factor or enzyme). In some instances, an object sequence comprises one or more of a nucleic acid sequence encoding a selectable marker (e.g., an auxotrophic marker or an antibiotic marker), and/or a nucleic acid control element (e.g., a promoter, enhancer, silencer, or insulator).

Parapalindromic: As used herein, the term parapalindromic refers to a property of a pair of nucleic acid sequences, wherein one of the nucleic acid sequences is either a palindrome relative to the other nucleic acid sequence, or has at least 50% (e.g., at least 50%, 55%, 60%, 65%, 70%, 75%, 80%, 85%, 90%, 95%, 96%, 97%, 98%, 99%, or 100%) sequence identity to a palindrome relative to the other nucleic acid sequence, or has no more than 1, 2, 3, 4, 5, 6, 7, or 8 sequence mismatches relative to the other nucleic acid sequence. “Parapalindromic sequences,” as used herein, refer to at least one of a pair of nucleic acid sequences that are parapalindromic relative to each other. A “parapalindromic region,” as used herein, refers to a nucleic acid sequence, or the portions thereof, that comprise two parapalindromic sequences. In some instances, a parapalindromic region comprises two paralindromic sequences flanking a nucleic acid segment, e.g., comprising a core sequence.

### BRIEF DESCRIPTION OF THE FIGURES

FIG. 1 shows a diagram of an exemplary recombinase reporter plasmid. An inactive reporter plasmid containing an inverted GFP gene flanked by recombinase recognition sites (e.g., loxP) in inverted orientation can be activated by the presence of a cognate recombinase (e.g., Cre), which results in flipping of the GFP gene into an orientation in which transcription of the coding sequence is driven by the upstream promoter (e.g., CMV).

FIG. 2 shows diagrams describing exemplary recombinase-mediated integration into the human genome. In the top diagram, a recombinase expressed from the recombinase expression plasmid recognizes a first target site on the insert DNA plasmid and a second target site in the human genome and catalyzes recombination between these two sites, resulting in integration of the insert DNA plasmid into the human genome at the second target site. In the bottom diagram, primer and probe positions for a ddPCR assay to quantify genomic integration events are shown.

### DETAILED DESCRIPTION

This disclosure relates to compositions, systems and methods for targeting, editing, modifying or manipulating a DNA sequence (e.g., inserting a heterologous object DNA sequence into a target site of a mammalian genome) at one or more locations in a DNA sequence in a cell, tissue or subject, e.g., in vivo or in vitro. The object DNA sequence may include, e.g., a coding sequence, a regulatory sequence, a gene expression unit.

### Gene-Writer™ Genome Editors

The present invention provides recombinase polypeptides (e.g., tyrosine recombinase polypeptides, e.g., as listed in Table 1 or 2) that can be used to modify or manipulate a DNA sequence, e.g., by recombining two DNA sequences comprising cognate recognition sequences that can be bound by the recombinase polypeptide. A Gene Writer™ gene editor system may, in some embodiments, comprise: (A) a polypeptide or a nucleic acid encoding a polypeptide, wherein the polypeptide comprises (i) a domain that contains recombinase activity, and (ii) a domain that contains DNA binding functionality (e.g., a DNA recognition domain that, for example, binds to or is capable of binding to a recognition sequence, e.g., as described herein); and (B) an insert DNA comprising (i) a sequence that binds the polypeptide (e.g., a recognition sequence as described herein) and, optionally, (ii) an object sequence (e.g., a heterologous object sequence). In some embodiments, the domain that contains recombinase activity and the domain that contains DNA binding functionality is the same domain. For example, the Gene Writer genome editor protein may comprise a DNA-binding domain and a recombinase domain. In certain embodiments, the elements of the Gene Writer™ gene editor polypeptide can be derived from sequences of a recombinase polypeptide (e.g., a tyrosine recombinase), e.g., as described herein, e.g., as listed in Table 1 or 2. In some embodiments the Gene Writer genome editor is combined with a second polypeptide. In some embodiments the second polypeptide is derived from a recombinase polypeptide (e.g., a tyrosine recombinase), e.g., as described herein, e.g., as listed in Table 1 or 2.

### Recombinase Polypeptide Component of Gene Writer Gene Editor System

An exemplary family of recombinase polypeptides that can be used in the systems, cells, and methods described herein includes the tyrosine recombinases. Generally, tyrosine recombinases are enzymes that catalyze site-specific recombination between two recognition sequences. The two recognition sequences may be, e.g., on the same nucleic acid (e.g., DNA) molecule, or may be present in two separate nucleic acid (e.g., DNA) molecules. In some embodiments, a tyrosine recombinase polypeptide comprises two domains, an N-terminal domain that comprises DNA contact sites, and a C-terminal domain that comprises the active site.

Tyrosine recombinases generally operate by concomitant binding of two recombinase polypeptide monomers to each of the recognition sequences, such that four monomers are involved in a single recombinase reaction. As described, for example, in Gaj et al. (2014; Biotechnol. Bioeng. 111(1): 1-15; incorporated herein by reference in its entirety), after binding of each pair of tyrosine recombinase monomers to the recognition sequences, the DNA-bound dimers then undergo DNA strand breaks, strand exchange, and rejoining to form Holliday junction intermediates, followed by an additional round of DNA strand breaks and ligation to form the recombined strands. Non-limiting examples of tyrosine recombinase include Cre recombinase and Flp recombinase, as well as the recombinase polypeptides listed in Table 1 or 2.

A skilled artisan can determine the nucleic acid and corresponding polypeptide sequences of a recombinase polypeptide (e.g., tyrosine recombinase) and domains thereof, e.g., by using routine sequence analysis tools as Basic Local Alignment Search Tool (BLAST) or CD-Search for conserved domain analysis. Other sequence analysis tools are known and can be found, e.g., at https://molbiol-tools.ca, for example, at https://molbiol-tools.ca/Motifs.htm.

Exemplary Recombinase Polypeptides

In some embodiments, a Gene Writer™ gene editor system comprises a recombinase polypeptide (e.g., a tyrosine recombinase polypeptide), e.g., as described herein. Generally, a recombinase polypeptide (e.g., a tyrosine recombinase polypeptide) specifically binds to a nucleic acid recognition sequence and catalyzes a recombination reaction at a site within the recognition sequence (e.g., a core sequence within the recognition sequence). In some embodiments, a recombinase polypeptide catalyzes recombination between a recognition sequence, or a portion thereof (e.g., a core sequence thereof) and another nucleic acid sequence (e.g., an insert DNA comprising a cognate recognition sequence and, optionally, an object sequence, e.g., a heterologous object sequence). For example, a recombinase polypeptide (e.g., a tyrosine recombinase polypeptide) may catalyze a recombination reaction that results in insertion of an object sequence, or a portion thereof, into another nucleic acid molecule (e.g., a genomic DNA molecule, e.g., a chromosome or mitochondrial DNA).

Table 1 below provides exemplary bidirectional tyrosine recombinase polypeptide amino acid sequences (see column 1), and their corresponding DNA recognition sequences (see columns 2 and 3), which were identified bioinformatically. Tables 1 and 2 comprise amino acid sequences that had not previously been identified as bidirectional tyrosine recombinases, and also includes corresponding DNA recognition sequences of tyrosine recombinases for which the DNA recognition sequences were previously unknown. The amino acid sequence of each accession number in column 1 of Table 1 is hereby incorporated by reference in its entirety.

More specifically, column 2 provides the native DNA recognition sequence (e.g., from bacteria or archaea), and column 3 provides a corresponding human DNA recognition sequence for the recombinase listed in that row. Column 4 indicates the genomic location of the human DNA recognition sequence of column 3. Column 5 provides the safe harbor score of the human DNA recognition sequence, indicating the number of safe harbor criteria met by the site.

The DNA recognition sequences of Table 1 have the following domains: a first parapalindromic sequence, a core sequence, and a second parapalindromic sequence. Without wishing to be bound by theory, in some embodiments, a tyrosine recombinase recognizes a DNA recognition sequence based on the parapalindromic region (the first and second parapalindromic sequences), and does not have any particular sequence requirements for the core sequence. Thus, in some embodiments, a tyrosine recombinase can insert DNA into a target site in the human genome, wherein the target site has a core sequence that may diverge substantially or completely from the native core sequence. Consequently, Table 1, column 2 includes Ns in these positions. In some embodiments, a core overlap sequence in an insert DNA may be chosen to match, at least partially, the corresponding sequence in the human genome. In some embodiments the recombinase only has a single human DNA recognition sequence.

TABLE 1
Exemplary tyrosine recombinases, corresponding recognition sequences, human
genomic locations thereof, and safe harbor score of the genomic location. As listed in the
DNA sequences, “N” can be any nucleotide (e.g., any one of A, C, G, or T).

| 1. | | | | | 4. Genomic | |
|---|---|---|---|---|---|---|
| Bidirectional | SEQ | 2. Native DNA | SEQ | 3. Human DNA | location of | 5. Safe |
| Tyrosine | ID | recognition | ID | recognition | human DNA | Harbor |
| Recombinase | NO: | sequence | NO: | sequence | sequence | Score |
| WP_0067171 | 1 | AATAAAGGGAATNN | 608 | AATAAAGGGAATAT | chr1:186448978- | 3 |
| 73.1 | | NNNNNNATTCCCTTT | | CTTATCATTCCCTTT | 186449009 | |
| | | ATT | | ATT | | |
| WP_0067185 | 2 | AATAAAGGGAATNN | 609 | AATAAAGGGAATAT | chr1:186448978- | 3 |
| 80.1 | | NNNNNNATTCCCTTT | | CTTATCATTCCCTTT | 186449009 | |
| | | ATT | | ATT | | |
| WP_0067192 | 3 | AATAAAGGGAATNN | 610 | AATAAAGGGAATAT | chr1:186448978- | 3 |
| 34.1 | | NNNNNNATTCCCTTT | | CTTATCATTCCCTTT | 186449009 | |
| | | ATT | | ATT | | |
| WP_1098591 | 4 | AATAAAGGGAATNN | 611 | AATAAAGGGAATAT | chr1:186448978- | 3 |
| 98.1 | | NNNNNNATTCCCTTT | | CTTATCATTCCCTTT | 186449009 | |
| | | ATT | | ATT | | |
| WP_0067171 | 5 | AATAAAGGGAATNN | 612 | AATAAAGGGAATAT | chr1:186448978- | 3 |
| 95.1 | | NNNNNNATTCCCTTT | | CTTATCATTCCCTTT | 186449009 | |
| | | ATT | | ATT | | |
| WP_0057157 | 6 | AATAAAGGGAATNN | 613 | AATAAAGGGAATAT | chr1:186448978- | 3 |
| 99.1 | | NNNNNNATTCCCTTT | | CTTATCATTCCCTTT | 186449009 | |
| | | ATT | | ATT | | |
| WP_1201665 | 7 | TTTTTTTGTATTNNNN | 614 | TTTTTTTGTATTTAA | chr15:98195234- | 5 |
| 65.1 | | NNNNNAAAAGAAAA | | AGAGGCAAAAGAA | 98195266 | |
| | | AAA | | AAAAA | | |
| WP_0613297 | 8 | TCTCTATATATANNN | 615 | TCTCTATATATATAT | chr18:34123564- | 5 |
| 56.1 | | NNNNNTATATATAGA | | GAGAATATATATAG | 34123595 | |
| | | GA | | AGA | | |
| WP_0104972 | 9 | AAAAATAAAACTGNN | 616 | AAAAATAAAACTGG | chr20:31321773- | 5 |
| 71.1 | | NNNNNNNTAGTTTTA | | GAAAAAAATAGTTT | 31321807 | |
| | | TTTTT | | TATTTTT | | |
| WP_0381509 | 10 | CACTGATATATANNN | 617 | CACTGATATATATC | chr3:164894717- | 6 |
| 96.1 | | NNNNTATATATCAGT | | ACTGATATATATCA | 164894747 | |
| | | G | | GTG | | |
| WP_0381508 | 11 | CACTGATATATANNN | 618 | CACTGATATATATC | chr3:164894717- | 6 |
| 98.1 | | NNNNTATATATCAGT | | ACTGATATATATCA | 164894747 | |
| | | G | | GTG | | |
| WP_0177400 | 12 | CAATTTTTGAAANNN | 619 | CAATTTTTGAAATTT | chr4:127054362- | 4 |
| 00.1 | | NNNNTTTCAAAAATT | | TCAATTTCAAAAATT | 127054392 | |
| | | G | | G | | |
| WP_0177442 | 13 | CAATTTTTGAAANNN | 620 | CAATTTTTGAAATTT | chr4:127054362- | 4 |
| 57.1 | | NNNNTTTCAAAAATT | | TCAATTTCAAAAATT | 127054392 | |
| | | G | | G | | |
| WP_0177461 | 14 | CAATTTTTGAAANNN | 621 | CAATTTTTGAAATTT | chr4:127054362- | 4 |
| 51.1 | | NNNNTTTCAAAAATT | | TCAATTTCAAAAATT | 127054392 | |
| | | G | | G | | |
| WP_1260450 | 15 | TAATGTTCTATANNN | 622 | TAATGTTCTATAATG | chr4:13893338- | 5 |
| 42.1 | | NNNNNTATAAAACAC | | TGGTTTATAAAACA | 13893369 | |
| | | TA | | CTA | | |
| XP_0123333 | 16 | TGCATATACATANNN | 623 | TGCATATACATATAT | chr5:127323005- | 6 |
| 05.1 | | NNNNNTATATATATG | | ATGCATATATATAT | 127323036 | |
| | | TA | | GTA | | |
| WP_0730250 | 17 | TTATGTCCAATANNN | 624 | TTATGTCCAATATAA | chr1:88050039- | 7 |
| 39.1 | | NNNNNTATTGGACAT | | AGCTATATTGGACA | 88050070 | |
| | | AG | | TAA | | |
| WP_0076355 | 18 | TTATGTCCAATANNN | 625 | TTATGTCCAATATAA | chr1:88050039- | 7 |
| 52.1 | | NNNNNTATTGGACAT | | AGCTATATTGGACA | 88050070 | |
| | | AG | | TAA | | |
| WP_0589581 | 19 | TGACTTCGTATANNN | 626 | TGACTTCGTATAAT | chr1:106584230- | 6 |
| 35.1 | | NNNNNTATACGAAGC | | AAACTTTATAGGAG | 106584261 | |
| | | CA | | GCCA | | |
| WP_0909670 | 20 | TGACTTCGTATANNN | 627 | TGACTTCGTATAAT | chr1:106584230- | 6 |
| 54.1 | | NNNNNTATACGAAGC | | AAACTTTATAGGAG | 106584261 | |
| | | CA | | GCCA | | |
| WP_0103653 | 21 | TAATGTCCAATANNN | 628 | TTATGTCCAATATAA | chr1:88050039- | 7 |
| 36.1 | | NNNNNTATCGGACAT | | AGCTATATTGGACA | 88050070 | |
| | | AA | | TAA | | |
| WP_0163928 | 22 | GACCACTCCAGANNN | 629 | GACCACTTCAGACA | chr13:80495061- | 7 |
| 93.1 | | NNNNNNTCTGGAGT | | AGATTGGTCTGGAA | 80495093 | |
| | | GGTG | | TGGTG | | |
| WP_0478245 | 23 | GGACATGTGATANNN | 630 | GGACATGTGATAAT | chr15:73681757- | 7 |
| 97.1 | | NNNNNTATCACATGT | | TCAATTTTGCACATG | 73681788 | |
| | | TG | | TTG | | |
| WP_0464074 | 24 | GCACTAGCGATANNN | 631 | GCACTAGCTATAGG | chr18:26615767- | 7 |
| 94.1 | | NNNNNTATCACTAGT | | AATTGGGATCACTA | 26615798 | |
| | | GC | | GTGC | | |
| WP_0037125 | 25 | CCCCTAACTAGANNN | 632 | CCCCTAATTAGAAC | chr2:211644330- | 6 |
| 23.1 | | NNNNTCTAATTAGGG | | ACATTTCTAATTATG | 211644360 | |
| | | G | | GG | | |
| WP_0050276 | 26 | CAGCCTCTTAGANNN | 633 | CAGCCTCTTAGCAA | chr3:39477201- | 7 |
| 58.1 | | NNNNTCTAAGGGGCT | | AAATTTTTAAGGGG | 39477231 | |
| | | T | | CTT | | |
| WP_0211703 | 27 | TAACTAATGATANNN | 634 | TAACTAGTGATAGA | chr5:110266294- | 7 |
| 77.1 | | NNNNNNTATCACTAG | | TAACAGTTATCACT | 110266326 | |
| | | TTG | | AGTTA | | |
| WP_0151699 | 28 | CTAAAGTAAGAGANN | 635 | CTGAAGTAAGAAAT | chr8:82693106- | 6 |
| 02.1 | | NNNNNNTTTCTTACT | | TTGCAAATTTCTTAC | 82693139 | |
| | | TCAG | | TTCAG | | |
| WP_0894151 | 29 | ATGACTTCGTATANN | 636 | ATGACTTCGTATAA | chr1:106584229- | 6 |
| 06.1 | | NNNNNNTATACGAA | | TAAACTTTATAGGA | 106584262 | |
| | | GTCAT | | GGCCAT | | |
| WP_0226242 | 30 | TGACTTCGTATANNN | 637 | TGACTTCGTATAAT | chr1:106584230- | 6 |
| 68.1 | | NNNNNTATACGAAGT | | AAACTTTATAGGAG | 106584261 | |
| | | CA | | GCCA | | |
| WP_0461030 | 31 | TGACTTCGTATANNN | 638 | TGACTTCGTATAAT | chr1:106584230- | 6 |
| 89.1 | | NNNNNTATACGAAGT | | AAACTTTATAGGAG | 106584261 | |
| | | CA | | GCCA | | |
| WP_0690271 | 32 | TGACTTCGTATANNN | 639 | TGACTTCGTATAAT | chr1:106584230- | 6 |
| 20.1 | | NNNNNTATACGAAGT | | AAACTTTATAGGAG | 106584261 | |
| | | CA | | GCCA | | |
| WP_0106719 | 33 | TGACTTCGTATANNN | 640 | TGACTTCGTATAAT | chr1:106584230- | 6 |
| 27.1 | | NNNNNTATACGAAGT | | AAACTTTATAGGAG | 106584261 | |
| | | CA | | GCCA | | |
| WP_1096537 | 34 | TGACTTCGTATANNN | 641 | TGACTTCGTATAAT | chr1:106584230- | 6 |
| 47.1 | | NNNNNTATACGAAGT | | AAACTTTATAGGAG | 106584261 | |
| | | CA | | GCCA | | |
| WP_1341619 | 35 | TGACTTCGTATANNN | 642 | TGACTTCGTATAAT | chr1:106584230- | 6 |
| 39.1 | | NNNNNTATACGAAGT | | AAACTTTATAGGAG | 106584261 | |
| | | CA | | GCCA | | |
| WP_1115348 | 36 | TGACTTCGTATANNN | 643 | TGACTTCGTATAAT | chr1:106584230- | 6 |
| 63.1 | | NNNNNTATACGAAGT | | AAACTTTATAGGAG | 106584261 | |
| | | CA | | GCCA | | |
| WP_1280855 | 37 | TGACTTCGTATANNN | 644 | TGACTTCGTATAAT | chr1:106584230- | 6 |
| 08.1 | | NNNNNTATACGAAGT | | AAACTTTATAGGAG | 106584261 | |
| | | CA | | GCCA | | |
| WP_1157646 | 38 | TGACTTCGTATANNN | 645 | TGACTTCGTATAAT | chr1:106584230- | 6 |
| 42.1 | | NNNNNTATACGAAGT | | AAACTTTATAGGAG | 106584261 | |
| | | CA | | GCCA | | |
| WP_1111383 | 39 | TGACTTCGTATANNN | 646 | TGACTTCGTATAAT | chr1:106584230- | 6 |
| 05.1 | | NNNNNTATACGAAGT | | AAACTTTATAGGAG | 106584261 | |
| | | CA | | GCCA | | |
| WP_0088397 | 40 | TCATGTCCGATANNN | 647 | TCATGACCTATATAC | chr1:165167590- | 5 |
| 47.1 | | NNNNNNTACCGGAC | | TTCTGGTACCAGAC | 165167622 | |
| | | ATAA | | ATAA | | |
| WP_0654178 | 41 | GATTTTTTTAACANNN | 648 | GATTTTTTTAACAAA | chr1:170443548- | 6 |
| 88.1 | | NNNNNNTATTATAAA | | AAATATATAATTAA | 170443582 | |
| | | AATC | | AAAATC | | |
| WP_0584139 | 42 | TGAGACGGGATANN | 649 | TGAGACTGCATAAA | chr1:190843617- | 6 |
| 92.1 | | NNNNNNNTATCCCAT | | TTATAAATATCCTAT | 190843649 | |
| | | CTGA | | CTGA | | |
| WP_0992351 | 43 | TGAGACGGGATANN | 650 | TGAGACTGCATAAA | chr1:190843617- | 6 |
| 64.1 | | NNNNNNNTATCCCAT | | TTATAAATATCCTAT | 190843649 | |
| | | CTGA | | CTGA | | |
| WP_0031395 | 44 | AAGCCATAGACANNN | 651 | AAGCCATAAAGATG | chr1:208272467- | 6 |
| 53.1 | | NNNNNTGTGTATGGC | | GGGCCTTGTGTCTG | 208272498 | |
| | | TT | | GCTT | | |
| WP_1328984 | 45 | GCTTGGTGCACANNN | 652 | GCATAGTGCACATT | chr1:212042241- | 7 |
| 17.1 | | NNNNTGTGACCCAAG | | AGACCTCTGACCCA | 212042271 | |
| | | C | | AGC | | |
| WP_1208099 | 46 | AAAAGCGTGATANNN | 653 | CAAAGCAGGATATT | chr1:214115937- | 5 |
| 06.1 | | NNNNNNTATCACGCC | | ATCAGGCTATCACG | 214115969 | |
| | | TTT | | CCTTT | | |
| WP_0757581 | 47 | CCGGCGCAAACANNN | 654 | CCGGCGCAGAAAG | chr1:21651977- | 4 |
| 85.1 | | NNNNNTGTTTGCGCC | | GGCCGCTTGTTCGC | 21652008 | |
| | | GC | | GCCGC | | |
| WP_0633139 | 48 | TGGCAAGCTATANNN | 655 | TGGCAAGCTATAAA | chr1:217009498- | 6 |
| 27.1 | | NNNNNNTATATCTTG | | ACAAGCATAAAACT | 217009530 | |
| | | CCA | | TCCCA | | |
| WP_0382026 | 49 | AAAGAAGCGATANN | 656 | AAAGAAGTGATAA | chr1:218206501- | 7 |
| 23.1 | | NNNNNNNTATCGCTT | | GAATTATTCATCTCT | 218206533 | |
| | | TTTT | | TTTTT | | |
| WP_1105609 | 50 | CTACTTCCGATANNN | 657 | CTCCTTCCAATAAA | chr1:236983188- | 6 |
| 45.1 | | NNNNNTGTCGGAAG | | GCCTTGTGTTGGAA | 236983219 | |
| | | TAG | | GTAG | | |
| WP_1023257 | 51 | CTACTTCCGATANNN | 658 | CTCCTTCCAATAAA | chr1:236983188- | 6 |
| 37.1 | | NNNNNTGTCGGAAG | | GCCTTGTGTTGGAA | 236983219 | |
| | | TAG | | GTAG | | |
| WP_1100959 | 52 | CTACTTCCGATANNN | 659 | CTCCTTCCAATAAA | chr1:236983188- | 6 |
| 79.1 | | NNNNNTGTCGGAAG | | GCCTTGTGTTGGAA | 236983219 | |
| | | TAG | | GTAG | | |
| WP_0141069 | 53 | CTACTTCCGATANNN | 660 | CTCCTTCCAATAAA | chr1:236983188- | 6 |
| 07.1 | | NNNNNTGTCGGAAG | | GCCTTGTGTTGGAA | 236983219 | |
| | | TAG | | GTAG | | |
| WP_0704062 | 54 | CTACTTCCGATANNN | 661 | CTCCTTCCAATAAA | chr1:236983188- | 6 |
| 27.1 | | NNNNNTGTCGGAAG | | GCCTTGTGTTGGAA | 236983219 | |
| | | TAG | | GTAG | | |
| WP_0396836 | 55 | TCTATATCCCATANNN | 662 | TCTATATACTATATA | chr1:239232551- | 6 |
| 93.1 | | NNNNNTATAGGATAT | | TAAGTATATAGTAT | 239232584 | |
| | | AGA | | ATAGA | | |
| WP_0581019 | 56 | ATTAGTCCCACANNN | 663 | TTTAGTCCCACAAAT | chr1:240346758- | 4 |
| 78.1 | | NNNNNNTGTGTGACT | | TTAAAATATGTGAC | 240346790 | |
| | | ACT | | TGCT | | |
| WP_0732883 | 57 | TTTAGGTATCATANN | 664 | TTTAGGCATCATGA | chr1:37227820- | 6 |
| 22.1 | | NNNNNNNTATGATG | | TGCTGGCATATGAT | 37227854 | |
| | | CCTAAA | | CCCTAAA | | |
| WP_1029063 | 58 | TTAGGTCTCATANNN | 665 | TTAGGTCTCTTTTTA | chr1:44815049- | 5 |
| 31.1 | | NNNNNTATGAGACCT | | CCTTGTAAGAGACC | 44815080 | |
| | | TA | | TTA | | |
| WP_0455723 | 59 | TCACTGTCCATANNN | 666 | TCACTGTCCTTATCT | chr1:58905291- | 7 |
| 21.1 | | NNNNCATGGACAGT | | ACAACATGGAGATT | 58905321 | |
| | | GA | | GA | | |
| WP_0413384 | 60 | CAATGTCCAATANNN | 667 | TTATGTCCAATATAA | chr1:88050039- | 7 |
| 71.1 | | NNNNNTATTGGACAT | | AGCTATATTGGACA | 88050070 | |
| | | TA | | TAA | | |
| WP_0110437 | 61 | CTATGTCCGATANNN | 668 | TTATGTCCAATATAA | chr1:88050039- | 7 |
| 09.1 | | NNNNNTATTGGACAT | | AGCTATATTGGACA | 88050070 | |
| | | AG | | TAA | | |
| WP_0417369 | 62 | CTATGTCCGATANNN | 669 | TTATGTCCAATATAA | chr1:88050039- | 7 |
| 50.1 | | NNNNNTATTGGACAT | | AGCTATATTGGACA | 88050070 | |
| | | AG | | TAA | | |
| WP_0703749 | 63 | CTATGTCCGATANNN | 670 | TTATGTCCAATATAA | chr1:88050039- | 7 |
| 86.1 | | NNNNNTATTGGACAT | | AGCTATATTGGACA | 88050070 | |
| | | AG | | TAA | | |
| WP_0330821 | 64 | CTATGTCCGATANNN | 671 | TTATGTCCAATATAA | chr1:88050039- | 7 |
| 29.1 | | NNNNNTATTGGACAT | | AGCTATATTGGACA | 88050070 | |
| | | AG | | TAA | | |
| WP_0571809 | 65 | CTATGTCCGATANNN | 672 | TTATGTCCAATATAA | chr1:88050039- | 7 |
| 66.1 | | NNNNNTATTGGACAT | | AGCTATATTGGACA | 88050070 | |
| | | AG | | TAA | | |
| WP_0517439 | 66 | TTATGTCCGATANNN | 673 | TTATGTCCAATATAA | chr1:88050039- | 7 |
| 15.1 | | NNNNNTATCGGACAT | | AGCTATATTGGACA | 88050070 | |
| | | AT | | TAA | | |
| WP_0725989 | 67 | TTATGTCCGATANNN | 674 | TTATGTCCAATATAA | chr1:88050039- | 7 |
| 06.1 | | NNNNNTCTCGGACAT | | AGCTATATTGGACA | 88050070 | |
| | | AA | | TAA | | |
| WP_0693376 | 68 | TTATGTCCGATANNN | 675 | TTATGTCCAATATAA | chr1:88050039- | 7 |
| 75.1 | | NNNNNTCTCGGACAT | | AGCTATATTGGACA | 88050070 | |
| | | AA | | TAA | | |
| WP_0607342 | 69 | GCTTGCGACATANNN | 676 | GGTTGCGACATACA | chr1:94419447- | 5 |
| 94.1 | | NNNNNTATGTCGCAA | | GGTATGTATGTCAC | 94419478 | |
| | | AC | | ATAC | | |
| WP_0363653 | 70 | TTTGTTGGTATANNN | 677 | TTTGAGGGTATTTA | chr1:99638466- | NA |
| 62.1 | | NNNNNTATACCAACA | | TTTTGCTATACCAAC | 99638497 | |
| | | AA | | AAA | | |
| WP_0886525 | 71 | CTATGTCCAATANNN | 678 | CTATGTACATTATCT | chr10:107928889- | 5 |
| 86.1 | | NNNNNNTATTGGAC | | TATATTTATTGGACA | 107928921 | |
| | | ATGA | | TGT | | |
| PLX79396.1 | 72 | TCAGCCGGAAGANN | 679 | TCAGCCGGAAGGTG | chr10:111439026- | 6 |
| | | NNNNNTCTTGCGGCT | | GAACTTCTGGCAGC | 111439056 | |
| | | GC | | TGC | | |
| WP_0128527 | 73 | AAACCCTACAGANNN | 680 | AAACCCTACAGAAT | chr10:112359538- | 4 |
| 32.1 | | NNNNNTCTGTAGGGT | | TGTACTTCTGAAGG | 112359569 | |
| | | TA | | ATCA | | |
| WP_0128527 | 74 | AAACCCTACAGANNN | 681 | AAACCCTACAGAAT | chr10:112359538- | 4 |
| 33.1 | | NNNNNTCTGTAGGGT | | TGTACTTCTGAAGG | 112359569 | |
| | | TA | | ATCA | | |
| WP_0659354 | 75 | TTAGGTCTGATANNN | 682 | TTAGGTCTGATATA | chr10:120864993- | 5 |
| 87.1 | | NNNNNNTATCCGACC | | AATGAAGTCTTTGA | 120865025 | |
| | | CAA | | CCCAA | | |
| WP_0104523 | 76 | TCACATGGGATANNN | 683 | TTACTTGGGATACA | chr10:121206254- | 6 |
| 01.1 | | NNNNNNTACCCCGTG | | AAATCTGTACCCAG | 121206286 | |
| | | TGA | | TGTGA | | |
| WP_0902087 | 77 | TCACATGGGATANNN | 684 | TTACTTGGGATACA | chr10:121206254- | 6 |
| 26.1 | | NNNNNNTACCCCGTG | | AAATCTGTACCCAG | 121206286 | |
| | | TGA | | TGTGA | | |
| WP_0621521 | 78 | TCATCGTACATANNN | 685 | TCCTCTTACATACTT | chr10:131836361- | 5 |
| 19.1 | | NNNNNTATGTATGAT | | TAAAATATGTATGA | 131836392 | |
| | | GA | | TTA | | |
| WP_0131963 | 79 | TATGACTCCAGANNN | 686 | TATGACTTCAAACT | chr10:32990424- | 7 |
| 26.1 | | NNNNNNTCTGGAGT | | GTTATTCTCTGGAG | 32990456 | |
| | | CACA | | TCATA | | |
| WP_0135778 | 80 | TATGACTCCAGANNN | 687 | TATGACTTCAAACT | chr10:32990424- | 7 |
| 22.1 | | NNNNNNTCTGGAGT | | GTTATTCTCTGGAG | 32990456 | |
| | | CACA | | TCATA | | |
| WP_0393899 | 81 | TATGACTCCAGANNN | 688 | TATGACTTCAAACT | chr10:32990424- | 7 |
| 14.1 | | NNNNNNTCTGGAGT | | GTTATTCTCTGGAG | 32990456 | |
| | | CACA | | TCATA | | |
| WP_0337689 | 82 | TGTGACTCCAGANNN | 689 | TATGACTTCAAACT | chr10:32990424- | 7 |
| 26.1 | | NNNNNNTCTGGAGT | | GTTATTCTCTGGAG | 32990456 | |
| | | CATA | | TCATA | | |
| WP_0567737 | 83 | TGTGACTCCAGANNN | 690 | TATGACTTCAAACT | chr10:32990424- | 7 |
| 90.1 | | NNNNNNTCTGGAGT | | GTTATTCTCTGGAG | 32990456 | |
| | | CATA | | TCATA | | |
| WP_0120758 | 84 | TTAAGTCTGATANNN | 691 | TTAAGTCAAATATCT | chr10:60537494- | 6 |
| 09.1 | | NNNNNNTATCCGACC | | ACTAGATATCCCAC | 60537526 | |
| | | TAA | | CTAA | | |
| WP_0339867 | 85 | TTAAGTCTGATANNN | 692 | TTAAGTCAAATATCT | chr10:60537494- | 6 |
| 89.1 | | NNNNNNTATCCGACC | | ACTAGATATCCCAC | 60537526 | |
| | | TAA | | CTAA | | |
| WP_0057522 | 86 | TTGCAAGGAACANNN | 693 | TTGCAAGGAACTGT | chr10:61854428- | 5 |
| 18.1 | | NNNNNTGCTCCTTGC | | TAAGAATTTTCCTTG | 61854459 | |
| | | AT | | CAT | | |
| WP_0112718 | 87 | TTGCAAGGAACANNN | 694 | TTGCAAGGAACTGT | chr10:61854428- | 5 |
| 67.1 | | NNNNNTGCTCCTTGC | | TAAGAATTTTCCTTG | 61854459 | |
| | | AT | | CAT | | |
| WP_0694813 | 88 | CTTATTAATTAATANN | 695 | CTTGATAATTAATA | chr10:63808356- | 7 |
| 44.1 | | NNNNNTATTAATTAA | | ATGAGGTTATTAAT | 63808390 | |
| | | TAAG | | TAATAAT | | |
| WP_0928377 | 89 | TCACTCACGATANNN | 696 | TCACCCACGTCACC | chr10:86883137- | 4 |
| 35.1 | | NNNNNNTATCGTGG | | CTTGGATTATCGTG | 86883169 | |
| | | GTAA | | GGTAA | | |
| WP_0572029 | 90 | TTACCCACGATANNN | 697 | TCACCCACGTCACC | chr10:86883137- | 4 |
| 84.1 | | NNNNNNTATCGTGG | | CTTGGATTATCGTG | 86883169 | |
| | | GTAA | | GGTAA | | |
| WP_0572675 | 91 | TTACCCACGATANNN | 698 | TCACCCACGTCACC | chr10:86883137- | 4 |
| 49.1 | | NNNNNNTATCGTGG | | CTTGGATTATCGTG | 86883169 | |
| | | GTAA | | GGTAA | | |
| WP_0770196 | 92 | TACGGGGAAAGANN | 699 | TAGGAGGAAAGAC | chr11:100278888- | 5 |
| 34.1 | | NNNNNTCTTTCCCCG | | TTTCAGTCTTTCCCC | 100278918 | |
| | | TT | | ATT | | |
| WP_0837688 | 93 | TCAAGATGAACANNN | 700 | TCAAGATGAACAAA | chr11:134140724- | 5 |
| 87.1 | | NNNNNNTGTTTATCT | | CCACATATGTGTTTT | 134140756 | |
| | | TGA | | TTGA | | |
| ACZ42745.1 | 94 | TCAAGATGAACANNN | 701 | TCAAGATGAACAAA | chr11:134140724- | 5 |
| | | NNNNNNTGTTTATCT | | CCACATATGTGTTTT | 134140756 | |
| | | TGA | | TTGA | | |
| WP_0590616 | 95 | TTAACTTGAATANNN | 702 | TTAATTTGAATATAA | chr11:21310918- | 6 |
| 37.1 | | NNNNNCATTCAAGCT | | TCTGTCATTCAAGTT | 21310949 | |
| | | AA | | GA | | |
| WP_0569745 | 96 | AATCGTTGATATANN | 703 | AATCATTCATATATA | chr11:39698382- | 6 |
| 19.1 | | NNNNNNTATATTAAC | | TATATATATATTAAC | 39698415 | |
| | | GTTT | | ATTT | | |
| WP_0033308 | 97 | AACAAGAGCAGANN | 704 | AACAGGAACACACA | chr11:72593387- | 6 |
| 82.1 | | NNNNNNCCTGCTCTT | | CTTACACCTGCTCTT | 72593418 | |
| | | GCT | | GCT | | |
| WP_0008767 | 98 | TGAGTATTTATATAN | 705 | TGAGTATTTATATAT | chr11:95634315- | 6 |
| 35.1 | | NNNNNNNTATGTAA | | ACTTGAGTATATAT | 95634350 | |
| | | ATACTCA | | ATACACA | | |
| WP_0198215 | 99 | TGATCGATAACANNN | 706 | TGATCAATAACACC | chr11:98224565- | 5 |
| 68.1 | | NNNNTGTTATCGATT | | AAGCCTGTCATCAA | 98224595 | |
| | | A | | TTA | | |
| WP_0112393 | 100 | TTACATTCGATANNN | 707 | TTAGATTCAATATTT | chr12:103480844- | 4 |
| 95.1 | | NNNNNNTATCGGAT | | TTGAATTATTGGAT | 103480876 | |
| | | GTAA | | GTAA | | |
| WP_0136957 | 101 | TTACTTCCGATANNN | 708 | TTACATCTGATAAG | chr12:105057007- | 5 |
| 83.1 | | NNNNNNTATCGGAA | | GATCTAGTATCGAA | 105057039 | |
| | | ATAT | | AATAT | | |
| YP_0091255 | 102 | GCCCTGGTCAGANNN | 709 | GCCCTGGTGACAGG | chr12:119742033- | 7 |
| 17.1 | | NNNNNTCTGACCGG | | GGAGTCTCTGACCT | 119742064 | |
| | | GGC | | GGGC | | |
| WP_0620417 | 103 | GCGTGACGCAGANN | 710 | GCGTGAGGAAGAG | chr12:15116187- | 6 |
| 33.1 | | NNNNNNNTCTGCGTC | | CAGCCCATTCTGCA | 15116219 | |
| | | ACGC | | TCACGC | | |
| WP_0448784 | 104 | CACCTCCAAATANNN | 711 | AACCCCCAAATAGT | chr12:23398673- | 4 |
| 38.1 | | NNNNNNTATTAGGA | | TAACCTATATTAGG | 23398705 | |
| | | GGTC | | TGGTC | | |
| KPU82353.1 | 105 | TTATTTCCGATANNN | 712 | TTATTTCCTATATTT | chr12:29882634- | 7 |
| | | NNNNNNTATCGGAA | | TAAGTTTATAAGAA | 29882666 | |
| | | AAAA | | AAAA | | |
| WP_0484992 | 106 | ATLTTTGTCAGANNN | 713 | ATATTTGTCAGAAA | chr12:30608656- | 7 |
| 02.1 | | NNNNCCCGACAAAG | | AAAAATCTGACAAA | 30608686 | |
| | | AT | | GAT | | |
| YP_195916.1 | 107 | TCTATGGACATANNN | 714 | TCTATGTACATAGG | chr12:31904100- | 6 |
| | | NNNNNAATGTCCATA | | TATGTCTATGTACAT | 31904131 | |
| | | GA | | AGA | | |
| WP_0133971 | 108 | TCTATGGACATANNN | 715 | TCTATGTACATAGG | chr12:31904100- | 6 |
| 05.1 | | NNNNNAATGTCCATA | | TATGTCTATGTACAT | 31904131 | |
| | | GA | | AGA | | |
| WP_0575912 | 109 | TCTATGGACATANNN | 716 | TCTATGTACATAGG | chr12:31904100- | 6 |
| 91.1 | | NNNNNAATGTCCATA | | TATGTCTATGTACAT | 31904131 | |
| | | GA | | AGA | | |
| WP_1140706 | 110 | ATTAGTTATGATANN | 717 | ATTAGTTATGATAA | chr12:33682974- | 4 |
| 45.1 | | NNNNNNNTATCGTA | | ATATGACATAACAC | 33683008 | |
| | | AGTAAT | | AAGTAAT | | |
| WP_1201285 | 111 | TAGAAAGCCATANNN | 718 | AAGAAAGCCATGG | chr12:48381088- | 7 |
| 27.1 | | NNNNNNTATGGCTTC | | ACATCAATTATGGC | 48381120 | |
| | | CTG | | TTCATG | | |
| WP_0147866 | 112 | TTACCTCCGACANNN | 719 | TTCCCTCAGACAAT | chr12:50098705- | 6 |
| 80.1 | | NNNNNTGTCGTGGG | | GACTGATGTGGTGG | 50098736 | |
| | | TAA | | GTAA | | |
| WP_0656537 | 113 | TTACTTCCGATANNN | 720 | GTACTTCCCATAGG | chr12:53017915- | 5 |
| 36.1 | | NNNNNTATCGGAAG | | TGTTGGTATCTGAA | 53017946 | |
| | | TAG | | GTAC | | |
| WP_0823040 | 114 | TTACTTCCGATANNN | 721 | GTACTTCCCATAGG | chr12:53017915- | 5 |
| 40.1 | | NNNNNTATCGGAAG | | TGTTGGTATCTGAA | 53017946 | |
| | | TAC | | GTAC | | |
| WP_0767290 | 115 | CAACGTCTGATANNN | 722 | CTAAGTCTGATAGG | chr12:61149603- | 7 |
| 31.1 | | NNNNNNTATCAGAC | | ACTTTTTTATCAGAC | 61149635 | |
| | | GTAG | | TTAG | | |
| WP_0123298 | 116 | CAACGTCTGATANNN | 723 | CTAAGTCTGATAGG | chr12:61149603- | 7 |
| 41.1 | | NNNNNNTATCAGAC | | ACTTTTTTATCAGAC | 61149635 | |
| | | GTAG | | TTAG | | |
| KIU27889.1 | 117 | CAACGTCTGATANNN | 724 | CTAAGTCTGATAGG | chr12:61149603- | 7 |
| | | NNNNNNTATCAGAC | | ACTTTTTTATCAGAC | 61149635 | |
| | | GTAG | | TTAG | | |
| WP_0293617 | 118 | CTACGTCTGATANNN | 725 | CTAAGTCTGATAGG | chr12:61149603- | 7 |
| 46.1 | | NNNNNNTATCAGAC | | ACTTTTTTATCAGAC | 61149635 | |
| | | GTTG | | TTAG | | |
| WP_0123298 | 119 | CTACGTCTGATANNN | 726 | CTAAGTCTGATAGG | chr12:61149603- | 7 |
| 56.1 | | NNNNNNTATCAGAC | | ACTTTTTTATCAGAC | 61149635 | |
| | | GTTG | | TTAG | | |
| WP_0120104 | 120 | AAGCATGACACANNN | 727 | AAGCATGAAACAGA | chr12:69370960- | 5 |
| 52.1 | | NNNNCGTGCCATGCT | | ATGTAAGTGCCATG | 69370990 | |
| | | T | | CAT | | |
| WP_0853611 | 121 | TAGGTATTGATANNN | 728 | TAGGTATTGATATG | chr12:89090193- | 5 |
| 67.1 | | NNNNNTCTCACTACC | | GTTTGGTGTCCCTA | 89090224 | |
| | | TA | | CCCA | | |
| WP_0078582 | 122 | AAATACCACAGANNN | 729 | AAATAACACAGCAA | chr12:90787740- | 6 |
| 08.1 | | NNNNNTCTGCGGTAC | | CTCCACTCTGGGGT | 90787771 | |
| | | TT | | ACTT | | |
| WP_0460272 | 123 | TTAGGTTGGATANNN | 730 | TTAGGTTGGCTAAG | chr13:54916637- | 7 |
| 27.1 | | NNNNNNTATCAGACC | | ATAAGAAAATCAGA | 54916669 | |
| | | TAA | | CCAAA | | |
| OUV98802.1 | 124 | ATTACTATTGATANN | 731 | AATAATATTGATAT | chr13:63134582- | 5 |
| | | NNNNNNNTATCATTA | | CAACTAATTATCATC | 63134616 | |
| | | GTAAT | | AGTAAT | | |
| WP_0755008 | 125 | ATTACTATTGATANN | 732 | AATAATATTGATAT | chr13:63134582- | 5 |
| 61.1 | | NNNNNNNTATCATTA | | CAACTAATTATCATC | 63134616 | |
| | | GTAAT | | AGTAAT | | |
| WP_0119065 | 126 | GATAACAAGATANNN | 733 | TATAACAAGATACA | chr13:75289152- | 6 |
| 04.1 | | NNNNNNTATCTTGTT | | GCCTGTTTATCTTG | 75289184 | |
| | | ATC | | GTATA | | |
| WP_0142690 | 127 | TATCCAATGTATANN | 734 | TATACATTGTATATA | chr13:82628490- | 6 |
| 99.1 | | NNNNNNNTATACATT | | CATTGTATATACATT | 82628524 | |
| | | GGATA | | GTATA | | |
| WP_0023288 | 128 | GGAAAACGTAGANN | 735 | GGAAAACTTAGAAA | chr13:84656932- | 6 |
| 98.1 | | NNNNNNTCTACGTTT | | GAATCTTCCACTTTT | 84656963 | |
| | | TCC | | TCC | | |
| WP_0512794 | 129 | CTAGTCATGATANNN | 736 | GTAGTCATGATATT | chr13:93786373- | 5 |
| 02.1 | | NNNNNTATCGTGACT | | TCTTACTATTATGAC | 93786404 | |
| | | AT | | TAT | | |
| WP_0580022 | 130 | CCTTAATAGACANNN | 737 | CACTAATAGACATA | chr14:102832746- | 5 |
| 97.1 | | NNNNNNTATCTATTA | | GCAGTAATATATAT | 102832778 | |
| | | AGC | | TAAGC | | |
| WP_0140808 | 131 | GGTGCAACCACANNN | 738 | GGTGCCACCACATG | chr14:105806860- | 7 |
| 79.1 | | NNNNTGTGGCTGCAC | | TCATGTATGGCTGC | 105806890 | |
| | | C | | CCC | | |
| WP_0344654 | 132 | CTTTCGGACAGANNN | 739 | CGTTGGGACAGATG | chr14:106294549- | 5 |
| 37.1 | | NNNNNTATGTCTGAA | | TGTGTACATGTCTG | 106294580 | |
| | | AG | | AAAG | | |
| WP_0150459 | 133 | TAATCCGTAATANNN | 740 | TAATCCTTAATACTA | chr14:37532388- | 6 |
| 88.1 | | NNNNTTTAACGGATT | | ACACTTTAACGCAT | 37532418 | |
| | | A | | AA | | |
| WP_1254404 | 134 | TTAGTACCGATANNN | 741 | TTACTACCAATATAA | chr14:52339287- | 6 |
| 93.1 | | NNNNNNTATCGGTAC | | CAACACTACCAGTA | 52339319 | |
| | | TAA | | CTAA | | |
| TDN36797.1 | 135 | TTAGTACCGATANNN | 742 | TTACTACCAATATAA | chr14:52339287- | 6 |
| | | NNNNNNTATCAGTAC | | CAACACTACCAGTA | 52339319 | |
| | | TAA | | CTAA | | |
| WP_1336591 | 136 | TTAGTACCGATANNN | 743 | TTACTACCAATATAA | chr14:52339287- | 6 |
| 53.1 | | NNNNNNTATCAGTAC | | CAACACTACCAGTA | 52339319 | |
| | | TAA | | CTAA | | |
| OUW60929.1 | 137 | TTTTTTCCGATANNNN | 744 | TTTTTTCCTATAGTT | chr14:63944046- | NA |
| | | NNNNNTATCGGAAAT | | TTCTGGTATTTGAA | 63944078 | |
| | | AT | | ATAT | | |
| WP_0089163 | 138 | AAAGTACCAACANNN | 745 | AAAGGACCAACTTT | chr14:66956028- | 5 |
| 47.1 | | NNNNTGTTGATACTT | | GATTTTGTTGATTCT | 66956058 | |
| | | T | | TT | | |
| WP_0168003 | 139 | CAAAAGGCGACANN | 746 | CAAATGTAGACAGT | chr14:67334559- | 6 |
| 55.1 | | NNNNNTGTCGCCTTT | | TTATATGTCGCCTTT | 67334589 | |
| | | TT | | TT | | |
| WP_0292037 | 140 | CAAAAGGCGACANN | 747 | CAAATGTAGACAGT | chr14:67334559- | 6 |
| 06.1 | | NNNNNTGTCGCCTTT | | TTATATGTCGCCTTT | 67334589 | |
| | | TT | | TT | | |
| WP_0300647 | 141 | TGACTCCTGATANNN | 748 | TAACTCCTGGTAAA | chr14:71732258- | 6 |
| 47.1 | | NNNNNTCTCTGGAGT | | CAGGTCTTTCTGGA | 71732289 | |
| | | CA | | GTCA | | |
| WP_0484742 | 142 | CCGTCATGGATANNN | 749 | CCGTCATGGGGCTT | chr14:93647060- | 6 |
| 44.1 | | NNNNNTATCCATGAA | | ATAGTCTATCCATG | 93647091 | |
| | | GC | | AAGC | | |
| WP_1093140 | 143 | TTACACATGATANNN | 750 | TTATACATGATATAC | chr14:94716806- | 5 |
| 41.1 | | NNNNNNTATCATGTG | | ATAACATATCATGT | 94716838 | |
| | | TAA | | ATTA | | |
| WP_0292243 | 144 | CAAAAGGCGACANN | 751 | CAAAAGGAGACAG | chr14:97951200- | 7 |
| 90.1 | | NNNNNNTGTCGCCTT | | GCATATTTTTCCCCT | 97951231 | |
| | | TTT | | TTTT | | |
| WP_0106467 | 145 | CAAAAGGCGACANN | 752 | CAAAAGGAGACAG | chr14:97951200- | 7 |
| 15.1 | | NNNNNNTGTCGCCTT | | GCATATTTTTCCCCT | 97951231 | |
| | | TTT | | TTTT | | |
| WP_0217104 | 146 | CAAAAGGCGACANN | 753 | CAAAAGGAGACAG | chr14:97951200- | 7 |
| 15.1 | | NNNNNNTGTCGCCTT | | GCATATTTTTCCCCT | 97951231 | |
| | | TTT | | TTTT | | |
| WP_0119992 | 147 | CAAAAGGCGACANN | 754 | CAAAAGGAGACAG | chr14:97951200- | 7 |
| 82.1 | | NNNNNNTGTCGCCTT | | GCATATTTTTCCCCT | 97951231 | |
| | | TTT | | TTTT | | |
| WP_0506492 | 148 | CAAAAGGCGACANN | 755 | CAAAAGGAGACAG | chr14:97951200- | 7 |
| 39.1 | | NNNNNNTGTCGCCTT | | GCATATTTTTCCCCT | 97951231 | |
| | | TTT | | TTTT | | |
| WP_0519410 | 149 | TTGAGTGCTACANNN | 756 | CTGGGTGCTCCAGG | chr15:23506248- | 6 |
| 91.1 | | NNNNNNTGTAGCACT | | GGCTCTCTGTAGCA | 23506280 | |
| | | CAA | | CTCAA | | |
| WP_0653470 | 150 | TTGAGTGCTACANNN | 757 | CTGGGTGCTCCAGG | chr15:23506248- | 6 |
| 10.1 | | NNNNNNTGTAGCACT | | GGCTCTCTGTAGCA | 23506280 | |
| | | CAA | | CTCAA | | |
| WP_0496814 | 151 | GAACCCTTGATANNN | 758 | GAACACTTTATAAG | chr15:43410177- | 6 |
| 75.1 | | NNNNTATCAAGGGTT | | TTATATATGAAGGG | 43410207 | |
| | | T | | TTT | | |
| WP_0253152 | 152 | AACAGATCAATANNN | 759 | AAAAGATCAATAAA | chr15:47468716- | 6 |
| 61.1 | | NNNNGATTGATCTGT | | GCACAGATTGAATT | 47468746 | |
| | | T | | GTT | | |
| WP_0380697 | 153 | TTATGTCCAATANNN | 760 | TTATTTCCAATAAAT | chr15:54938190- | 8 |
| 93.1 | | NNNNNNTATCGGAC | | CAGAATTATAGCAC | 54938222 | |
| | | ATGA | | ATGA | | |
| WP_0068610 | 154 | AACAACCACATANNN | 761 | AAAAACCACATATT | chr15:58808569- | 6 |
| 39.1 | | NNNNNTATGTGGTTG | | ATAAAATATATGGT | 58808600 | |
| | | TT | | TTTT | | |
| WP_1023690 | 155 | TCAGATGGGATANNN | 762 | TCAGTTGGGATACA | chr15:90749251- | 7 |
| 17.1 | | NNNNNNTATCCCGTG | | ATTAATGTAACCTG | 90749283 | |
| | | TGA | | TGTGA | | |
| WP_0032125 | 156 | TCAGATGGGATANNN | 763 | TCAGTTGGGATACA | chr15:90749251- | 7 |
| 74.1 | | NNNNNNTATCCCGTG | | ATTAATGTAACCTG | 90749283 | |
| | | TGA | | TGTGA | | |
| WP_1026049 | 157 | TCAGATGGGATANNN | 764 | TCAGTTGGGATACA | chr15:90749251- | 7 |
| 09.1 | | NNNNNNTATCCCGTG | | ATTAATGTAACCTG | 90749283 | |
| | | TGA | | TGTGA | | |
| WP_0084325 | 158 | TCAGATGGGATANNN | 765 | TCAGTTGGGATACA | chr15:90749251- | 7 |
| 17.1 | | NNNNNNTATCCCGTG | | ATTAATGTAACCTG | 90749283 | |
| | | TGA | | TGTGA | | |
| WP_0028923 | 159 | AAAATAGCGATANNN | 766 | AAAATAGGGATAAC | chr16:13245429- | 5 |
| 42.1 | | NNNNTATCGCTATTA | | AATAGTATCTCTATC | 13245459 | |
| | | T | | AT | | |
| WP_0028871 | 160 | AAAATAGCGATANNN | 767 | AAAATAGGGATAAC | chr16:13245429- | 5 |
| 64.1 | | NNNNTATCGCTATTA | | AATAGTATCTCTATC | 13245459 | |
| | | T | | AT | | |
| WP_0705783 | 161 | AAAATAGCGATANNN | 768 | AAAATAGGGATAAC | chr16:13245429- | 5 |
| 46.1 | | NNNNTATCGCTATTA | | AATAGTATCTCTATC | 13245459 | |
| | | T | | AT | | |
| WP_0115302 | 162 | CTACTCCGCAGANNN | 769 | CTCCTCCGCAGAAG | chr16:19016625- | 5 |
| 52.1 | | NNNNNTCTGCGGAG | | TCTGTGTCTGGGGA | 19016656 | |
| | | TAA | | GCAA | | |
| WP_0058340 | 163 | TTAGGGAGAAGANN | 770 | TTAGGGAGGAGAC | chr16:35081954- | 5 |
| 81.1 | | NNNNNNNTCTTCTCC | | AAGGCTGTTCTTTTC | 35081986 | |
| | | CTAC | | CCTCC | | |
| WP_1002941 | 164 | CAAGTATCGATANNN | 771 | CATGTATAGATATA | chr16:48917302- | 4 |
| 15.1 | | NNNNNTATCGATACT | | TATGCATATAGATA | 48917333 | |
| | | TA | | CTTA | | |
| WP_0412342 | 165 | CAAGTATCGATANNN | 772 | CATGTATAGATATA | chr16:48917302- | 4 |
| 71.1 | | NNNNNTATCGATACT | | TATGCATATAGATA | 48917333 | |
| | | TA | | CTTA | | |
| WP_0412020 | 166 | CAAGTATCGATANNN | 773 | CATGTATAGATATA | chr16:48917302- | 4 |
| 99.1 | | NNNNNTATCGATACT | | TATGCATATAGATA | 48917333 | |
| | | TA | | CTTA | | |
| WP_0888689 | 167 | CAAGTATCGATANNN | 774 | CATGTATAGATATA | chr16:48917302- | 4 |
| 73.1 | | NNNNNTATCGATACT | | TATGCATATAGATA | 48917333 | |
| | | TA | | CTTA | | |
| WP_0695548 | 168 | CAAGTATCGATANNN | 775 | CATGTATAGATATA | chr16:48917302- | 4 |
| 70.1 | | NNNNNTATCGATACT | | TATGCATATAGATA | 48917333 | |
| | | TA | | CTTA | | |
| WP_1032520 | 169 | CAAGTATCGATANNN | 776 | CATGTATAGATATA | chr16:48917302- | 4 |
| 06.1 | | NNNNNTATCGATACT | | TATGCATATAGATA | 48917333 | |
| | | TA | | CTTA | | |
| WP_1270056 | 170 | CAAGTATCGATANNN | 777 | CATGTATAGATATA | chr16:48917302- | 4 |
| 24.1 | | NNNNNTATCGATACT | | TATGCATATAGATA | 48917333 | |
| | | TA | | CTTA | | |
| SIQ01063.1 | 171 | CAAGTATCGATANNN | 778 | CATGTATAGATATA | chr16:48917302- | 4 |
| | | NNNNNTATCGATACT | | TATGCATATAGATA | 48917333 | |
| | | TA | | CTTA | | |
| WP_1006458 | 172 | CAAGTATCGATANNN | 779 | CATGTATAGATATA | chr16:48917302- | 4 |
| 80.1 | | NNNNNTATCGATACT | | TATGCATATAGATA | 48917333 | |
| | | TA | | CTTA | | |
| WP_1006537 | 173 | CAAGTATCGATANNN | 780 | CATGTATAGATATA | chr16:48917302- | 4 |
| 72.1 | | NNNNNTATCGATACT | | TATGCATATAGATA | 48917333 | |
| | | TA | | CTTA | | |
| WP_0419154 | 174 | CAAGTATCGATANNN | 781 | CATGTATAGATATA | chr16:48917302- | 4 |
| 08.1 | | NNNNNTATCGATACT | | TATGCATATAGATA | 48917333 | |
| | | TA | | CTTA | | |
| WP_1295040 | 175 | CAAGTATCGATANNN | 782 | CATGTATAGATATA | chr16:48917302- | 4 |
| 75.1 | | NNNNNTATCGATACT | | TATGCATATAGATA | 48917333 | |
| | | TA | | CTTA | | |
| WP_0946984 | 176 | CAAGTATCGATANNN | 783 | CATGTATAGATATA | chr16:48917302- | 4 |
| 59.1 | | NNNNNTATCGATACT | | TATGCATATAGATA | 48917333 | |
| | | TA | | CTTA | | |
| WP_1068867 | 177 | CAAGTATCGATANNN | 784 | CATGTATAGATATA | chr16:48917302- | 4 |
| 83.1 | | NNNNNTATCGATACT | | TATGCATATAGATA | 48917333 | |
| | | TA | | CTTA | | |
| WP_0177853 | 178 | CAAGTATCGATANNN | 785 | CATGTATAGATATA | chr16:48917302- | 4 |
| 58.1 | | NNNNNTATCGATACT | | TATGCATATAGATA | 48917333 | |
| | | TA | | CTTA | | |
| WP_1008583 | 179 | CAAGTATCGATANNN | 786 | CATGTATAGATATA | chr16:48917302- | 4 |
| 03.1 | | NNNNNTATCGATACT | | TATGCATATAGATA | 48917333 | |
| | | TA | | CTTA | | |
| WP_1232461 | 180 | CAAGTATCGATANNN | 787 | CATGTATAGATATA | chr16:48917302- | 4 |
| 39.1 | | NNNNNTATCGATACT | | TATGCATATAGATA | 48917333 | |
| | | TA | | CTTA | | |
| WP_0431627 | 181 | CAAGTATCGATANNN | 788 | CATGTATAGATATA | chr16:48917302- | 4 |
| 17.1 | | NNNNNTATCGATACT | | TATGCATATAGATA | 48917333 | |
| | | TA | | CTTA | | |
| WP_1242494 | 182 | CAAGTATCGATANNN | 789 | CATGTATAGATATA | chr16:48917302- | 4 |
| 52.1 | | NNNNNTATCGATACT | | TATGCATATAGATA | 48917333 | |
| | | TA | | CTTA | | |
| WP_0961195 | 183 | CAAGTATCGATANNN | 790 | CATGTATAGATATA | chr16:48917302- | 4 |
| 02.1 | | NNNNNTATCGATACT | | TATGCATATAGATA | 48917333 | |
| | | TA | | CTTA | | |
| WP_0842026 | 184 | CAAGTATCGATANNN | 791 | CATGTATAGATATA | chr16:48917302- | 4 |
| 52.1 | | NNNNNTATCGATACT | | TATGCATATAGATA | 48917333 | |
| | | TA | | CTTA | | |
| WP_0392158 | 185 | CAAGTATCGATANNN | 792 | CATGTATAGATATA | chr16:48917302- | 4 |
| 13.1 | | NNNNNTATCGATACT | | TATGCATATAGATA | 48917333 | |
| | | TA | | CTTA | | |
| WP_1242514 | 186 | CAAGTATCGATANNN | 793 | CATGTATAGATATA | chr16:48917302- | 4 |
| 91.1 | | NNNNNTATCGATACT | | TATGCATATAGATA | 48917333 | |
| | | TA | | CTTA | | |
| WP_0252017 | 187 | CAAGTATCGATANNN | 794 | CATGTATAGATATA | chr16:48917302- | 4 |
| 27.1 | | NNNNNTATCGATACT | | TATGCATATAGATA | 48917333 | |
| | | TA | | CTTA | | |
| WP_1257299 | 188 | CAAGTATCGATANNN | 795 | CATGTATAGATATA | chr16:48917302- | 4 |
| 07.1 | | NNNNNTATCGATACT | | TATGCATATAGATA | 48917333 | |
| | | TA | | CTTA | | |
| WP_0431229 | 189 | CAAGTATCGATANNN | 796 | CATGTATAGATATA | chr16:48917302- | 4 |
| 83.1 | | NNNNNTATCGATACT | | TATGCATATAGATA | 48917333 | |
| | | TA | | CTTA | | |
| WP_0733502 | 190 | CAAGTATCGATANNN | 797 | CATGTATAGATATA | chr16:48917302- | 4 |
| 84.1 | | NNNNNTATCGATACT | | TATGCATATAGATA | 48917333 | |
| | | TA | | CTTA | | |
| WP_1034707 | 191 | CAAGTATCGATANNN | 798 | CATGTATAGATATA | chr16:48917302- | 4 |
| 61.1 | | NNNNNTATCGATACT | | TATGCATATAGATA | 48917333 | |
| | | TA | | CTTA | | |
| WP_0431348 | 192 | CAAGTATCGATANNN | 799 | CATGTATAGATATA | chr16:48917302- | 4 |
| 01.1 | | NNNNNTATCGATACT | | TATGCATATAGATA | 48917333 | |
| | | TA | | CTTA | | |
| WP_1256066 | 193 | CAAGTATCGATANNN | 800 | CATGTATAGATATA | chr16:48917302- | 4 |
| 95.1 | | NNNNNTATCGATACT | | TATGCATATAGATA | 48917333 | |
| | | TA | | CTTA | | |
| WP_0989840 | 194 | CAAGTATCGATANNN | 801 | CATGTATAGATATA | chr16:48917302- | 4 |
| 54.1 | | NNNNNTATCGATACT | | TATGCATATAGATA | 48917333 | |
| | | TA | | CTTA | | |
| WP_1011491 | 195 | CAAGTATCGATANNN | 802 | CATGTATAGATATA | chr16:48917302- | 4 |
| 34.1 | | NNNNNTATCGATACT | | TATGCATATAGATA | 48917333 | |
| | | TA | | CTTA | | |
| WP_0877557 | 196 | CAAGTATCGATANNN | 803 | CATGTATAGATATA | chr16:48917302- | 4 |
| 18.1 | | NNNNNTATCGATACT | | TATGCATATAGATA | 48917333 | |
| | | TA | | CTTA | | |
| WP_0808913 | 197 | CAAGTATCGATANNN | 804 | CATGTATAGATATA | chr16:48917302- | 4 |
| 34.1 | | NNNNNTATCGATACT | | TATGCATATAGATA | 48917333 | |
| | | TA | | CTTA | | |
| WP_1115878 | 198 | CAAGTATCGATANNN | 805 | CATGTATAGATATA | chr16:48917302- | 4 |
| 63.1 | | NNNNNTATCGATACT | | TATGCATATAGATA | 48917333 | |
| | | TA | | CTTA | | |
| ABO90113.1 | 199 | CAAGTATCGATANNN | 806 | CATGTATAGATATA | chr16:48917302- | 4 |
| | | NNNNNTATCGATACT | | TATGCATATAGATA | 48917333 | |
| | | TA | | CTTA | | |
| WP_1032431 | 200 | CAAGTATCGATANNN | 807 | CATGTATAGATATA | chr16:48917302- | 4 |
| 21.1 | | NNNNNTATCGATACT | | TATGCATATAGATA | 48917333 | |
| | | TA | | CTTA | | |
| WP_1242438 | 201 | CAAGTATCGATANNN | 808 | CATGTATAGATATA | chr16:48917302- | 4 |
| 12.1 | | NNNNNTATCGATACT | | TATGCATATAGATA | 48917333 | |
| | | TA | | CTTA | | |
| WP_0428784 | 202 | CAAGTATCGATANNN | 809 | CATGTATAGATATA | chr16:48917302- | 4 |
| 86.1 | | NNNNNTATCGATACT | | TATGCATATAGATA | 48917333 | |
| | | TA | | CTTA | | |
| WP_0053470 | 203 | CAAGTATCGATANNN | 810 | CATGTATAGATATA | chr16:48917302- | 4 |
| 25.1 | | NNNNNTATCGATACT | | TATGCATATAGATA | 48917333 | |
| | | TA | | CTTA | | |
| WP_0420629 | 204 | CAAGTATCGATANNN | 811 | CATGTATAGATATA | chr16:48917302- | 4 |
| 22.1 | | NNNNNTATCGATACT | | TATGCATATAGATA | 48917333 | |
| | | TA | | CTTA | | |
| WP_0420550 | 205 | CAAGTATCGATANNN | 812 | CATGTATAGATATA | chr16:48917302- | 4 |
| 87.1 | | NNNNNTATCGATACT | | TATGCATATAGATA | 48917333 | |
| | | TA | | CTTA | | |
| WP_0751136 | 206 | CAAGTATCGATANNN | 813 | CATGTATAGATATA | chr16:48917302- | 4 |
| 48.1 | | NNNNNTATCGATACT | | TATGCATATAGATA | 48917333 | |
| | | TA | | CTTA | | |
| WP_0695268 | 207 | CAAGTATCGATANNN | 814 | CATGTATAGATATA | chr16:48917302- | 4 |
| 84.1 | | NNNNNTATCGATACT | | TATGCATATAGATA | 48917333 | |
| | | TA | | CTTA | | |
| WP_0505478 | 208 | CAAGTATCGATANNN | 815 | CATGTATAGATATA | chr16:48917302- | 4 |
| 38.1 | | NNNNNTATCGATACT | | TATGCATATAGATA | 48917333 | |
| | | TA | | CTTA | | |
| WP_0764917 | 209 | CAAGTATCGATANNN | 816 | CATGTATAGATATA | chr16:48917302- | 4 |
| 68.1 | | NNNNNTATCGATACT | | TATGCATATAGATA | 48917333 | |
| | | TA | | CTTA | | |
| SQH59660.1 | 210 | CAAGTATCGATANNN | 817 | CATGTATAGATATA | chr16:48917302- | 4 |
| | | NNNNNTATCGATACT | | TATGCATATAGATA | 48917333 | |
| | | TA | | CTTA | | |
| WP_0719101 | 211 | CAAGTATCGATANNN | 818 | CATGTATAGATATA | chr16:48917302- | 4 |
| 68.1 | | NNNNNTATCGATACT | | TATGCATATAGATA | 48917333 | |
| | | TA | | CTTA | | |
| OFC44115.1 | 212 | CAAGTATCGATANNN | 819 | CATGTATAGATATA | chr16:48917302- | 4 |
| | | NNNNNTATCGATACT | | TATGCATATAGATA | 48917333 | |
| | | TA | | CTTA | | |
| AHV35191.2 | 213 | CAAGTATCGATANNN | 820 | CATGTATAGATATA | chr16:48917302- | 4 |
| | | NNNNNTATCGATACT | | TATGCATATAGATA | 48917333 | |
| | | TA | | CTTA | | |
| EKB28734.1 | 214 | CAAGTATCGATANNN | 821 | CATGTATAGATATA | chr16:48917302- | 4 |
| | | NNNNNTATCGATACT | | TATGCATATAGATA | 48917333 | |
| | | TA | | CTTA | | |
| OCA67852.1 | 215 | CAAGTATCGATANNN | 822 | CATGTATAGATATA | chr16:48917302- | 4 |
| | | NNNNNTATCGATACT | | TATGCATATAGATA | 48917333 | |
| | | TA | | CTTA | | |
| KMK90327.1 | 216 | CAAGTATCGATANNN | 823 | CATGTATAGATATA | chr16:48917302- | 4 |
| | | NNNNNTATCGATACT | | TATGCATATAGATA | 48917333 | |
| | | TA | | CTTA | | |
| APJ17493.1 | 217 | CAAGTATCGATANNN | 824 | CATGTATAGATATA | chr16:48917302- | 4 |
| | | NNNNNTATCGATACT | | TATGCATATAGATA | 48917333 | |
| | | TA | | CTTA | | |
| WP_0591677 | 218 | CAAGTATCGATANNN | 825 | CATGTATAGATATA | chr16:48917302- | 4 |
| 96.1 | | NNNNNTATCGATACT | | TATGCATATAGATA | 48917333 | |
| | | TA | | CTTA | | |
| PKD25755.1 | 219 | CAAGTATCGATANNN | 826 | CATGTATAGATATA | chr16:48917302- | 4 |
| | | NNNNNTATCGATACT | | TATGCATATAGATA | 48917333 | |
| | | TA | | CTTA | | |
| WP_0521011 | 220 | CAAGTATCGATANNN | 827 | CATGTATAGATATA | chr16:48917302- | 4 |
| 92.1 | | NNNNNTATCGATACT | | TATGCATATAGATA | 48917333 | |
| | | TA | | CTTA | | |
| WP_0521590 | 221 | CAAGTATCGATANNN | 828 | CATGTATAGATATA | chr16:48917302- | 4 |
| 26.1 | | NNNNNTATCGATACT | | TATGCATATAGATA | 48917333 | |
| | | TA | | CTTA | | |
| AGM44110.1 | 222 | CAAGTATCGATANNN | 829 | CATGTATAGATATA | chr16:48917302- | 4 |
| | | NNNNNTATCGATACT | | TATGCATATAGATA | 48917333 | |
| | | TA | | CTTA | | |
| WP_0426547 | 223 | CAAGTATCGATANNN | 830 | CATGTATAGATATA | chr16:48917302- | 4 |
| 58.1 | | NNNNNTATCGATACT | | TATGCATATAGATA | 48917333 | |
| | | TA | | CTTA | | |
| WP_0426383 | 224 | CAAGTATCGATANNN | 831 | CATGTATAGATATA | chr16:48917302- | 4 |
| 08.1 | | NNNNNTATCGATACT | | TATGCATATAGATA | 48917333 | |
| | | TA | | CTTA | | |
| WP_0464007 | 225 | CAAGTATCGATANNN | 832 | CATGTATAGATATA | chr16:48917302- | 4 |
| 08.1 | | NNNNNTATCGATACT | | TATGCATATAGATA | 48917333 | |
| | | TA | | CTTA | | |
| ARW82171.1 | 226 | CAAGTATCGATANNN | 833 | CATGTATAGATATA | chr16:48917302- | 4 |
| | | NNNNNTATCGATACT | | TATGCATATAGATA | 48917333 | |
| | | TA | | CTTA | | |
| WP_0424673 | 227 | CAAGTATCGATANNN | 834 | CATGTATAGATATA | chr16:48917302- | 4 |
| 53.1 | | NNNNNTATCGATACT | | TATGCATATAGATA | 48917333 | |
| | | TA | | CTTA | | |
| WP_0511637 | 228 | CAAGTATCGATANNN | 835 | CATGTATAGATATA | chr16:48917302- | 4 |
| 65.1 | | NNNNNTATCGATACT | | TATGCATATAGATA | 48917333 | |
| | | TA | | CTTA | | |
| KOG94732.1 | 229 | CAAGTATCGATANNN | 836 | CATGTATAGATATA | chr16:48917302- | 4 |
| | | NNNNNTATCGATACT | | TATGCATATAGATA | 48917333 | |
| | | TA | | CTTA | | |
| EKB19089.1 | 230 | CAAGTATCGATANNN | 837 | CATGTATAGATATA | chr16:48917302- | 4 |
| | | NNNNNTATCGATACT | | TATGCATATAGATA | 48917333 | |
| | | TA | | CTTA | | |
| EKB18370.1 | 231 | CAAGTATCGATANNN | 838 | CATGTATAGATATA | chr16:48917302- | 4 |
| | | NNNNNTATCGATACT | | TATGCATATAGATA | 48917333 | |
| | | TA | | CTTA | | |
| WP_0820325 | 232 | CAAGTATCGATANNN | 839 | CATGTATAGATATA | chr16:48917302- | 4 |
| 88.1 | | NNNNNTATCGATACT | | TATGCATATAGATA | 48917333 | |
| | | TA | | CTTA | | |
| AEB50024.1 | 233 | CAAGTATCGATANNN | 840 | CATGTATAGATATA | chr16:48917302- | 4 |
| | | NNNNNTATCGATACT | | TATGCATATAGATA | 48917333 | |
| | | TA | | CTTA | | |
| EQC05143.1 | 234 | CAAGTATCGATANNN | 841 | CATGTATAGATATA | chr16:48917302- | 4 |
| | | NNNNNTATCGATACT | | TATGCATATAGATA | 48917333 | |
| | | TA | | CTTA | | |
| RAJ07841.1 | 235 | CAAGTATCGATANNN | 842 | CATGTATAGATATA | chr16:48917302- | 4 |
| | | NNNNNTATCGATACT | | TATGCATATAGATA | 48917333 | |
| | | TA | | CTTA | | |
| WP_1137395 | 236 | CAAGTATTGATANNN | 843 | CATGTATAGATATA | chr16:48917302- | 4 |
| 60.1 | | NNNNNTATCGATACT | | TATGCATATAGATA | 48917333 | |
| | | TA | | CTTA | | |
| WP_0615205 | 237 | CCAGCCCCTACANNN | 844 | CCAGCCCCTCCAGA | chr16:66346513- | 6 |
| 10.1 | | NNNNNTGTAGGGGC | | GAGCCCTGATGGG | 66346544 | |
| | | TGT | | GCTGT | | |
| WP_0069513 | 238 | TGCAAATATTACANN | 845 | TGCAAATTTTACAA | chr16:66394313- | 7 |
| 58.1 | | NNNNNNNTGTAATTT | | CCTTTACTTTTAATT | 66394347 | |
| | | TTGCA | | TTTCCA | | |
| WP_0400655 | 239 | TAAGTATCGATANNN | 846 | TAACTATCAATAGTT | chr17:10781706- | 6 |
| 15.1 | | NNNNNTATCGATACT | | ACTATTATCGATAG | 10781737 | |
| | | TG | | TTG | | |
| WP_1015315 | 240 | TAAGTATCGATANNN | 847 | TAACTATCAATAGTT | chr17:10781706- | 6 |
| 73.1 | | NNNNNTATCGATACT | | ACTATTATCGATAG | 10781737 | |
| | | TG | | TTG | | |
| WP_0412350 | 241 | TAAGTATCGATANNN | 848 | TAACTATCAATAGTT | chr17:10781706- | 6 |
| 50.1 | | NNNNNTATCGATACT | | ACTATTATCGATAG | 10781737 | |
| | | TG | | TTG | | |
| WP_0820386 | 242 | TAAGTATCGATANNN | 849 | TAACTATCAATAGTT | chr17:10781706- | 6 |
| 47.1 | | NNNNNTATCGATACT | | ACTATTATCGATAG | 10781737 | |
| | | TG | | TTG | | |
| WP_1085882 | 243 | TAAGTATCGATANNN | 850 | TAACTATCAATAGTT | chr17:10781706- | 6 |
| 31.1 | | NNNNNTATCGATACT | | ACTATTATCGATAG | 10781737 | |
| | | TG | | TTG | | |
| KRV94096.1 | 244 | TAAGTATCGATANNN | 851 | TAACTATCAATAGTT | chr17:10781706- | 6 |
| | | NNNNNTATCGATACT | | ACTATTATCGATAG | 10781737 | |
| | | TG | | TTG | | |
| WP_0993594 | 245 | TAAGTATCGATANNN | 852 | TAACTATCAATAGTT | chr17:10781706- | 6 |
| 35.1 | | NNNNNTATCGATACT | | ACTATTATCGATAG | 10781737 | |
| | | TG | | TTG | | |
| WP_1204142 | 246 | TAAGTATCGATANNN | 853 | TAACTATCAATAGTT | chr17:10781706- | 6 |
| 55.1 | | NNNNNTATCGATACT | | ACTATTATCGATAG | 10781737 | |
| | | TG | | TTG | | |
| WP_1013472 | 247 | TAAGTATCGATANNN | 854 | TAACTATCAATAGTT | chr17:10781706- | 6 |
| 86.1 | | NNNNNTATCGATACT | | ACTATTATCGATAG | 10781737 | |
| | | TG | | TTG | | |
| WP_1068436 | 248 | TAAGTATCGATANNN | 855 | TAACTATCAATAGTT | chr17:10781706- | 6 |
| 96.1 | | NNNNNTATCGATACT | | ACTATTATCGATAG | 10781737 | |
| | | TG | | TTG | | |
| WP_1242429 | 249 | TAAGTATCGATANNN | 856 | TAACTATCAATAGTT | chr17:10781706- | 6 |
| 06.1 | | NNNNNTATCGATACT | | ACTATTATCGATAG | 10781737 | |
| | | TG | | TTG | | |
| WP_0412027 | 250 | TAAGTATCGATANNN | 857 | TAACTATCAATAGTT | chr17:10781706- | 6 |
| 00.1 | | NNNNNTATCGATACT | | ACTATTATCGATAG | 10781737 | |
| | | TG | | TTG | | |
| WP_1231730 | 251 | TAAGTATCGATANNN | 858 | TAACTATCAATAGTT | chr17:10781706- | 6 |
| 50.1 | | NNNNNTATCGATACT | | ACTATTATCGATAG | 10781737 | |
| | | TG | | TTG | | |
| WP_1076829 | 252 | TAAGTATCGATANNN | 859 | TAACTATCAATAGTT | chr17:10781706- | 6 |
| 50.1 | | NNNNNTATCGATACT | | ACTATTATCGATAG | 10781737 | |
| | | TG | | TTG | | |
| WP_1288215 | 253 | TAAGTATCGATANNN | 860 | TAACTATCAATAGTT | chr17:10781706- | 6 |
| 47.1 | | NNNNNTATCGATACT | | ACTATTATCGATAG | 10781737 | |
| | | TG | | TTG | | |
| WP_0821806 | 254 | TAAGTATCGATANNN | 861 | TAACTATCAATAGTT | chr17:10781706- | 6 |
| 60.1 | | NNNNNTATCGATACT | | ACTATTATCGATAG | 10781737 | |
| | | TG | | TTG | | |
| WP_0820299 | 255 | TAAGTATCGATANNN | 862 | TAACTATCAATAGTT | chr17:10781706- | 6 |
| 42.1 | | NNNNNTATCGATACT | | ACTATTATCGATAG | 10781737 | |
| | | TG | | TTG | | |
| WP_0810132 | 256 | TAAGTATCGATANNN | 863 | TAACTATCAATAGTT | chr17:10781706- | 6 |
| 37.1 | | NNNNNTATCGATACT | | ACTATTATCGATAG | 10781737 | |
| | | TG | | TTG | | |
| WP_0249417 | 257 | TAAGTATCGATANNN | 864 | TAACTATCAATAGTT | chr17:10781706- | 6 |
| 85.1 | | NNNNNTATCGATACT | | ACTATTATCGATAG | 10781737 | |
| | | TG | | TTG | | |
| WP_0650175 | 258 | TAAGTATCGATANNN | 865 | TAACTATCAATAGTT | chr17:10781706- | 6 |
| 96.1 | | NNNNNTATCGATACT | | ACTATTATCGATAG | 10781737 | |
| | | TG | | TTG | | |
| WP_0428890 | 259 | TAAGTATCGATANNN | 866 | TAACTATCAATAGTT | chr17:10781706- | 6 |
| 28.1 | | NNNNNTATCGATACT | | ACTATTATCGATAG | 10781737 | |
| | | TG | | TTG | | |
| WP_1119106 | 260 | TAAGTATCGATANNN | 867 | TAACTATCAATAGTT | chr17:10781706- | 6 |
| 13.1 | | NNNNNTATCGATACT | | ACTATTATCGATAG | 10781737 | |
| | | TG | | TTG | | |
| WP_1268818 | 261 | TAAGTATCGATANNN | 868 | TAACTATCAATAGTT | chr17:10781706- | 6 |
| 46.1 | | NNNNNTATCGATACT | | ACTATTATCGATAG | 10781737 | |
| | | TG | | TTG | | |
| WP_0177790 | 262 | TAAGTATCGATANNN | 869 | TAACTATCAATAGTT | chr17:10781706- | 6 |
| 21.1 | | NNNNNTATCGATACT | | ACTATTATCGATAG | 10781737 | |
| | | TG | | TTG | | |
| WP_0807688 | 263 | TAAGTATCGATANNN | 870 | TAACTATCAATAGTT | chr17:10781706- | 6 |
| 65.1 | | NNNNNTATCGATACT | | ACTATTATCGATAG | 10781737 | |
| | | TG | | TTG | | |
| WP_0809731 | 264 | TAAGTATCGATANNN | 871 | TAACTATCAATAGTT | chr17:10781706- | 6 |
| 38.1 | | NNNNNTATCGATACT | | ACTATTATCGATAG | 10781737 | |
| | | TG | | TTG | | |
| WP_0249447 | 265 | TAAGTATCGATANNN | 872 | TAACTATCAATAGTT | chr17:10781706- | 6 |
| 68.1 | | NNNNNTATCGATACT | | ACTATTATCGATAG | 10781737 | |
| | | TG | | TTG | | |
| WP_1065525 | 266 | TAAGTATCGATANNN | 873 | TAACTATCAATAGTT | chr17:10781706- | 6 |
| 88.1 | | NNNNNTATCGATACT | | ACTATTATCGATAG | 10781737 | |
| | | TG | | TTG | | |
| WP_1139950 | 267 | TAAGTATCGATANNN | 874 | TAACTATCAATAGTT | chr17:10781706- | 6 |
| 02.1 | | NNNNNTATCGATACT | | ACTATTATCGATAG | 10781737 | |
| | | TG | | TTG | | |
| WP_1306323 | 268 | TAAGTATCGATANNN | 875 | TAACTATCAATAGTT | chr17:10781706- | 6 |
| 56.1 | | NNNNNTATCGATACT | | ACTATTATCGATAG | 10781737 | |
| | | TG | | TTG | | |
| WP_1137216 | 269 | TAAGTATCGATANNN | 876 | TAACTATCAATAGTT | chr17:10781706- | 6 |
| 56.1 | | NNNNNTATCGATACT | | ACTATTATCGATAG | 10781737 | |
| | | TG | | TTG | | |
| WP_0888462 | 270 | TAAGTATCGATANNN | 877 | TAACTATCAATAGTT | chr17:10781706- | 6 |
| 17.1 | | NNNNNTATCGATACT | | ACTATTATCGATAG | 10781737 | |
| | | TG | | TTG | | |
| WP_0763607 | 271 | TAAGTATCGATANNN | 878 | TAACTATCAATAGTT | chr17:10781706- | 6 |
| 55.1 | | NNNNNTATCGATACT | | ACTATTATCGATAG | 10781737 | |
| | | TG | | TTG | | |
| WP_1317306 | 272 | TAAGTATCGATANNN | 879 | TAACTATCAATAGTT | chr17:10781706- | 6 |
| 94.1 | | NNNNNTATCGATACT | | ACTATTATCGATAG | 10781737 | |
| | | TG | | TTG | | |
| WP_1032439 | 273 | TAAGTATCGATANNN | 880 | TAACTATCAATAGTT | chr17:10781706- | 6 |
| 80.1 | | NNNNNTATCGATACT | | ACTATTATCGATAG | 10781737 | |
| | | TG | | TTG | | |
| WP_0813046 | 274 | TAAGTATCGATANNN | 881 | TAACTATCAATAGTT | chr17:10781706- | 6 |
| 08.1 | | NNNNNTATCGATACT | | ACTATTATCGATAG | 10781737 | |
| | | TG | | TTG | | |
| WP_1188812 | 275 | TAAGTATCGATANNN | 882 | TAACTATCAATAGTT | chr17:10781706- | 6 |
| 29.1 | | NNNNNTATCGATACT | | ACTATTATCGATAG | 10781737 | |
| | | TG | | TTG | | |
| WP_0293008 | 276 | TAAGTATCGATANNN | 883 | TAACTATCAATAGTT | chr17:10781706- | 6 |
| 82.1 | | NNNNNTATCGATACT | | ACTATTATCGATAG | 10781737 | |
| | | TG | | TTG | | |
| WP_1029887 | 277 | TAAGTATCGATANNN | 884 | TAACTATCAATAGTT | chr17:10781706- | 6 |
| 85.1 | | NNNNNTATCGATACT | | ACTATTATCGATAG | 10781737 | |
| | | TG | | TTG | | |
| WP_0345236 | 278 | TAAGTATCGATANNN | 885 | TAACTATCAATAGTT | chr17:10781706- | 6 |
| 32.1 | | NNNNNTATCGATACT | | ACTATTATCGATAG | 10781737 | |
| | | TG | | TTG | | |
| WP_0117061 | 279 | TAAGTATCGATANNN | 886 | TAACTATCAATAGTT | chr17:10781706- | 6 |
| 13.1 | | NNNNNTATCGATACT | | ACTATTATCGATAG | 10781737 | |
| | | TG | | TTG | | |
| WP_0810861 | 280 | TAAGTATCGATANNN | 887 | TAACTATCAATAGTT | chr17:10781706- | 6 |
| 91.1 | | NNNNNTATCGATACT | | ACTATTATCGATAG | 10781737 | |
| | | TG | | TTG | | |
| WP_0457898 | 281 | TAAGTATCGATANNN | 888 | TAACTATCAATAGTT | chr17:10781706- | 6 |
| 55.1 | | NNNNNTATCGATACT | | ACTATTATCGATAG | 10781737 | |
| | | TG | | TTG | | |
| WP_1016174 | 282 | TAAGTATCGATANNN | 889 | TAACTATCAATAGTT | chr17:10781706- | 6 |
| 48.1 | | NNNNNTATCGATACT | | ACTATTATCGATAG | 10781737 | |
| | | TG | | TTG | | |
| WP_0999932 | 283 | TAAGTATCGATANNN | 890 | TAACTATCAATAGTT | chr17:10781706- | 6 |
| 15.1 | | NNNNNTATCGATACT | | ACTATTATCGATAG | 10781737 | |
| | | TG | | TTG | | |
| WP_1044559 | 284 | TAAGTATCGATANNN | 891 | TAACTATCAATAGTT | chr17:10781706- | 6 |
| 33.1 | | NNNNNTATCGATACT | | ACTATTATCGATAG | 10781737 | |
| | | TG | | TTG | | |
| WP_0428638 | 285 | TAAGTATCGATANNN | 892 | TAACTATCAATAGTT | chr17:10781706- | 6 |
| 72.1 | | NNNNNTATCGATACT | | ACTATTATCGATAG | 10781737 | |
| | | TG | | TTG | | |
| WP_0412057 | 286 | TAAGTATCGATANNN | 893 | TAACTATCAATAGTT | chr17:10781706- | 6 |
| 82.1 | | NNNNNTATCGATACT | | ACTATTATCGATAG | 10781737 | |
| | | TG | | TTG | | |
| WP_0431527 | 287 | TAAGTATCGATANNN | 894 | TAACTATCAATAGTT | chr17:10781706- | 6 |
| 10.1 | | NNNNNTATCGATACT | | ACTATTATCGATAG | 10781737 | |
| | | TG | | TTG | | |
| WP_1038589 | 288 | TAAGTATCGATANNN | 895 | TAACTATCAATAGTT | chr17:10781706- | 6 |
| 36.1 | | NNNNNTATCGATACT | | ACTATTATCGATAG | 10781737 | |
| | | TG | | TTG | | |
| WP_1242393 | 289 | TAAGTATCGATANNN | 896 | TAACTATCAATAGTT | chr17:10781706- | 6 |
| 32.1 | | NNNNNTATCGATACT | | ACTATTATCGATAG | 10781737 | |
| | | TG | | TTG | | |
| WP_1032618 | 290 | TAAGTATCGATANNN | 897 | TAACTATCAATAGTT | chr17:10781706- | 6 |
| 85.1 | | NNNNNTATCGATACT | | ACTATTATCGATAG | 10781737 | |
| | | TG | | TTG | | |
| WP_1032601 | 291 | TAAGTATCGATANNN | 898 | TAACTATCAATAGTT | chr17:10781706- | 6 |
| 30.1 | | NNNNNTATCGATACT | | ACTATTATCGATAG | 10781737 | |
| | | TG | | TTG | | |
| WP_1118092 | 292 | TAAGTATCGATANNN | 899 | TAACTATCAATAGTT | chr17:10781706- | 6 |
| 97.1 | | NNNNNTATCGATACT | | ACTATTATCGATAG | 10781737 | |
| | | TG | | TTG | | |
| WP_0813318 | 293 | TAAGTATCGATANNN | 900 | TAACTATCAATAGTT | chr17:10781706- | 6 |
| 71.1 | | NNNNNTATCGATACT | | ACTATTATCGATAG | 10781737 | |
| | | TG | | TTG | | |
| WP_0412151 | 294 | TAAGTATCGATANNN | 901 | TAACTATCAATAGTT | chr17:10781706- | 6 |
| 62.1 | | NNNNNTATCGATACT | | ACTATTATCGATAG | 10781737 | |
| | | TG | | TTG | | |
| WP_1266233 | 295 | TAAGTATCGATANNN | 902 | TAACTATCAATAGTT | chr17:10781706- | 6 |
| 23.1 | | NNNNNTATCGATACT | | ACTATTATCGATAG | 10781737 | |
| | | TG | | TTG | | |
| WP_0504900 | 296 | TAAGTATCGATANNN | 903 | TAACTATCAATAGTT | chr17:10781706- | 6 |
| 04.1 | | NNNNNTATCGATACT | | ACTATTATCGATAG | 10781737 | |
| | | TG | | TTG | | |
| WP_0420309 | 297 | TAAGTATCGATANNN | 904 | TAACTATCAATAGTT | chr17:10781706- | 6 |
| 57.1 | | NNNNNTATCGATACT | | ACTATTATCGATAG | 10781737 | |
| | | TG | | TTG | | |
| WP_0420832 | 298 | TAAGTATCGATANNN | 905 | TAACTATCAATAGTT | chr17:10781706- | 6 |
| 30.1 | | NNNNNTATCGATACT | | ACTATTATCGATAG | 10781737 | |
| | | TG | | TTG | | |
| WP_0643400 | 299 | TAAGTATCGATANNN | 906 | TAACTATCAATAGTT | chr17:10781706- | 6 |
| 28.1 | | NNNNNTATCGATACT | | ACTATTATCGATAG | 10781737 | |
| | | TG | | TTG | | |
| WP_0419807 | 300 | TAAGTATCGATANNN | 907 | TAACTATCAATAGTT | chr17:10781706- | 6 |
| 81.1 | | NNNNNTATCGATACT | | ACTATTATCGATAG | 10781737 | |
| | | TG | | TTG | | |
| WP_0426558 | 301 | TAAGTATCGATANNN | 908 | TAACTATCAATAGTT | chr17:10781706- | 6 |
| 14.1 | | NNNNNTATCGATACT | | ACTATTATCGATAG | 10781737 | |
| | | TG | | TTG | | |
| WP_0524471 | 302 | TAAGTATCGATANNN | 909 | TAACTATCAATAGTT | chr17:10781706- | 6 |
| 16.1 | | NNNNNTATCGATACT | | ACTATTATCGATAG | 10781737 | |
| | | TG | | TTG | | |
| PHS84353.1 | 303 | TAAGTATCGATANNN | 910 | TAACTATCAATAGTT | chr17:10781706- | 6 |
| | | NNNNNTATCGATACT | | ACTATTATCGATAG | 10781737 | |
| | | TG | | TTG | | |
| WP_0420378 | 304 | TAAGTATCGATANNN | 911 | TAACTATCAATAGTT | chr17:10781706- | 6 |
| 44.1 | | NNNNNTATCGATACT | | ACTATTATCGATAG | 10781737 | |
| | | TG | | TTG | | |
| OEG05223.1 | 305 | TAAGTATCGATANNN | 912 | TAACTATCAATAGTT | chr17:10781706- | 6 |
| | | NNNNNTATCGATACT | | ACTATTATCGATAG | 10781737 | |
| | | TG | | TTG | | |
| KLV47629.1 | 306 | TAAGTATCGATANNN | 913 | TAACTATCAATAGTT | chr17:10781706- | 6 |
| | | NNNNNTATCGATACT | | ACTATTATCGATAG | 10781737 | |
| | | TG | | TTG | | |
| AXV34415.1 | 307 | TAAGTATCGATANNN | 914 | TAACTATCAATAGTT | chr17:10781706- | 6 |
| | | NNNNNTATCGATACT | | ACTATTATCGATAG | 10781737 | |
| | | TG | | TTG | | |
| OCA59831.1 | 308 | TAAGTATCGATANNN | 915 | TAACTATCAATAGTT | chr17:10781706- | 6 |
| | | NNNNNTATCGATACT | | ACTATTATCGATAG | 10781737 | |
| | | TG | | TTG | | |
| SUU28072.1 | 309 | TAAGTATCGATANNN | 916 | TAACTATCAATAGTT | chr17:10781706- | 6 |
| | | NNNNNTATCGATACT | | ACTATTATCGATAG | 10781737 | |
| | | TG | | TTG | | |
| KWR69035.1 | 310 | TAAGTATCGATANNN | 917 | TAACTATCAATAGTT | chr17:10781706- | 6 |
| | | NNNNNTATCGATACT | | ACTATTATCGATAG | 10781737 | |
| | | TG | | TTG | | |
| WP_0524491 | 311 | TAAGTATCGATANNN | 918 | TAACTATCAATAGTT | chr17:10781706- | 6 |
| 73.1 | | NNNNNTATCGATACT | | ACTATTATCGATAG | 10781737 | |
| | | TG | | TTG | | |
| WP_0507171 | 312 | TAAGTATCGATANNN | 919 | TAACTATCAATAGTT | chr17:10781706- | 6 |
| 34.1 | | NNNNNTATCGATACT | | ACTATTATCGATAG | 10781737 | |
| | | TG | | TTG | | |
| OJW69670.1 | 313 | TAAGTATCGATANNN | 920 | TAACTATCAATAGTT | chr17:10781706- | 6 |
| | | NNNNNTATCGATACT | | ACTATTATCGATAG | 10781737 | |
| | | TG | | TTG | | |
| VEG96551.1 | 314 | TAAGTATCGATANNN | 921 | TAACTATCAATAGTT | chr17:10781706- | 6 |
| | | NNNNNTATCGATACT | | ACTATTATCGATAG | 10781737 | |
| | | TG | | TTG | | |
| WP_0842022 | 315 | TAAGTATCGATANNN | 922 | TAACTATCAATAGTT | chr17:10781706- | 6 |
| 79.1 | | NNNNNTATCGATACT | | ACTATTATCGATAG | 10781737 | |
| | | TG | | TTG | | |
| WP_0807412 | 316 | TAAGTATCGATANNN | 923 | TAACTATCAATAGTT | chr17:10781706- | 6 |
| 49.1 | | NNNNNTATCGATACT | | ACTATTATCGATAG | 10781737 | |
| | | TG | | TTG | | |
| EKB22195.1 | 317 | TAAGTATCGATANNN | 924 | TAACTATCAATAGTT | chr17:10781706- | 6 |
| | | NNNNNTATCGATACT | | ACTATTATCGATAG | 10781737 | |
| | | TG | | TTG | | |
| WP_0810429 | 318 | TAAGTATCGATANNN | 925 | TAACTATCAATAGTT | chr17:10781706- | 6 |
| 09.1 | | NNNNNTATCGATACT | | ACTATTATCGATAG | 10781737 | |
| | | TG | | TTG | | |
| EKB14410.1 | 319 | TAAGTATCGATANNN | 926 | TAACTATCAATAGTT | chr17:10781706- | 6 |
| | | NNNNNTATCGATACT | | ACTATTATCGATAG | 10781737 | |
| | | TG | | TTG | | |
| ANT70015.1 | 320 | TAAGTATCGATANNN | 927 | TAACTATCAATAGTT | chr17:10781706- | 6 |
| | | NNNNNTATCGATACT | | ACTATTATCGATAG | 10781737 | |
| | | TG | | TTG | | |
| EHI53752.1 | 321 | TAAGTATCGATANNN | 928 | TAACTATCAATAGTT | chr17:10781706- | 6 |
| | | NNNNNTATCGATACT | | ACTATTATCGATAG | 10781737 | |
| | | TG | | TTG | | |
| WP_0459721 | 322 | AGACACCTCAGANNN | 929 | GGACACCTCAAATC | chr17:19544976- | 7 |
| 72.1 | | NNNNNTCTGAGGTGT | | AGTCTCTCTGAGGA | 19545007 | |
| | | TT | | GTTT | | |
| WP_0736140 | 323 | CAAGAGATCACANNN | 930 | CAAGAGATCAAACT | chr17:54312893- | 4 |
| 59.1 | | NNNNTGTGGTCTCTT | | CCCCTTGTAGCCTCT | 54312923 | |
| | | T | | TT | | |
| WP_0605948 | 324 | GTGCCACAGATANNN | 931 | GTGCCACAGACATT | chr17:72851130- | 3 |
| 81.1 | | NNNNNNTATCCGTG | | CATGGGCCATCCGT | 72851162 | |
| | | GCAC | | AGCAC | | |
| WP_0617708 | 325 | GCTGATTTCAGANNN | 932 | GCTGAGTTGAGCCC | chr18:1279099- | 5 |
| 12.1 | | NNNNNTCTGAAATCA | | AGATCTTCTGAAAT | 1279130 | |
| | | TC | | CATC | | |
| WP_0759387 | 326 | TAAATAACGATANNN | 933 | AAAATAAAAATAAA | chr18:39171014- | 5 |
| 37.1 | | NNNNNNTATCGTTAT | | AATAATTTATCGTTA | 39171046 | |
| | | TTA | | TTTA | | |
| ETI84668.1 | 327 | TAAATAACGATANNN | 934 | AAAATAAAAATAAA | chr18:39171014- | 5 |
| | | NNNNNNTATCGTTAT | | AATAATTTATCGTTA | 39171046 | |
| | | TTA | | TTTA | | |
| WP_0997384 | 328 | TGACTATCGATANNN | 935 | TGACTATCGAAAAT | chr18:70607702- | 6 |
| 55.1 | | NNNNNNTATCGATAT | | TGGAAGAGATCGTT | 70607734 | |
| | | TTA | | ATTTA | | |
| WP_0660138 | 329 | TAATGTCCAATANNN | 936 | GAATGTCCAATAAT | chr19:11489967- | 6 |
| 27.1 | | NNNNNNTATCGGAC | | TCAATCCAATCTGA | 11489999 | |
| | | ATTA | | CATTA | | |
| WP_0061208 | 330 | GATAATAAGATANNN | 937 | GATAATAAGATAAG | chr19:23120611- | 3 |
| 90.1 | | NNNNNNCATCTTATT | | TGGTTATTATCTTAT | 23120643 | |
| | | ATC | | TAAA | | |
| PQV52181.1 | 331 | CAGCTATTGATANNN | 938 | TAGCTATTGATATTT | chr19:54357168- | 6 |
| | | NNNNNTATCAATAGT | | AAATTTATCCAAAG | 54357199 | |
| | | TG | | TTG | | |
| WP_1055081 | 332 | CAGCTATTGATANNN | 939 | TAGCTATTGATATTT | chr19:54357168- | 6 |
| 22.1 | | NNNNNTATCAATAGT | | AAATTTATCCAAAG | 54357199 | |
| | | TG | | TTG | | |
| EJT85494.1 | 333 | TCAGGTTCGAGANNN | 940 | TCAGGTTAGAGTTA | chr19:8046629- | 6 |
| | | NNNNNNTCTCGAAC | | ACCAAATTCTCGAA | 8046661 | |
| | | GTCA | | CATCA | | |
| WP_0354129 | 334 | CTACTTGTGATANNN | 941 | CTACTTGAGATATTT | chr2:112731169- | 5 |
| 14.1 | | NNNNNNTATCACAA | | TTCAGATAACACAA | 112731201 | |
| | | GTAG | | GTAT | | |
| WP_0053316 | 335 | AAAAGGTACTATANN | 942 | TAAAGCTACTATAC | chr2:126383828- | 6 |
| 70.1 | | NNNNNNNTATAGTA | | AGAGGAACTATAGT | 126383862 | |
| | | CCTTTT | | ACCATTT | | |
| WP_0107368 | 336 | ATACAATAGACANNN | 943 | ATACAATATACAAT | chr2:143143340- | 5 |
| 91.1 | | NNNNNAGCCTATTGT | | TAACATAGTATATT | 143143371 | |
| | | AT | | GTAT | | |
| WP_0107523 | 337 | ATACAATAGACANNN | 944 | ATACAATATACAAT | chr2:143143340- | 5 |
| 16.1 | | NNNNNAGCCTATTGT | | TAACATAGTATATT | 143143371 | |
| | | AT | | GTAT | | |
| PKP94160.1 | 338 | AGAGTGTTGATANNN | 945 | AGAGTGTTGATAAA | chr2:16118225- | 6 |
| | | NNNNNNTATCAACAC | | TTAGTGATATCAAG | 16118257 | |
| | | TAG | | TTTAG | | |
| WP_0149532 | 339 | ATTACTATCGATANN | 946 | ATTATTATCGATAAT | chr2:161938519- | 4 |
| 67.1 | | NNNNNNNTATCGTTA | | AATCTATTATCGATA | 161938553 | |
| | | GTAAT | | ATAAT | | |
| WP_0659972 | 340 | TAACTATCGATANNN | 947 | TTATTATCGATAATA | chr2:161938520- | 6 |
| 27.1 | | NNNNNNTATCGATAA | | ATCTATTATCGATAA | 161938552 | |
| | | TGA | | TAA | | |
| WP_0152415 | 341 | TCACTATCGATANNN | 948 | TTATTATCGATAATA | chr2:161938520- | 6 |
| 50.1 | | NNNNNNTATCGATAA | | ATCTATTATCGATAA | 161938552 | |
| | | TGA | | TAA | | |
| WP_1134800 | 342 | TCACTATCGATANNN | 949 | TTATTATCGATAATA | chr2:161938520- | 6 |
| 34.1 | | NNNNNNTATCGATAA | | ATCTATTATCGATAA | 161938552 | |
| | | TGA | | TAA | | |
| WP_1048400 | 343 | TCACTATCGATANNN | 950 | TTATTATCGATAATA | chr2:161938520- | 6 |
| 46.1 | | NNNNNNTATCGATA | | ATCTATTATCGATAA | 161938552 | |
| | | GTAA | | TAA | | |
| PZN95492.1 | 344 | TTACTATCGATANNN | 951 | TTATTATCGATAATA | chr2:161938520- | 6 |
| | | NNNNNNTATCGATA | | ATCTATTATCGATAA | 161938552 | |
| | | GTGA | | TAA | | |
| WP_0577957 | 345 | CTATGTCCAATANNN | 952 | ATATGTCCAATATG | chr2:166851262- | 5 |
| 42.1 | | NNNNNNTATCGGAC | | GGGTTAATATCTAA | 166851294 | |
| | | ATAT | | CATAT | | |
| WP_0894235 | 346 | CTATGTCCAATANNN | 953 | ATATGTCCAATATG | chr2:166851262- | 5 |
| 62.1 | | NNNNNNTATCGGAC | | GGGTTAATATCTAA | 166851294 | |
| | | ATAT | | CATAT | | |
| WP_0237219 | 347 | AAACGAATGATANNN | 954 | AAATAAATGATAGA | chr2:176201656- | 4 |
| 97.1 | | NNNNNNTATCATTCG | | TAAGGTCTATCATTC | 176201688 | |
| | | TTT | | ATTT | | |
| WP_0660522 | 348 | AAAACCTCCATANNN | 955 | AAACCCTGCATAAA | chr2:179830412- | 5 |
| 21.1 | | NNNNNNCATGGAGG | | AAATGATTATGGAG | 179830444 | |
| | | TTTT | | GTTTT | | |
| WP_0471389 | 349 | GGGCCCGCGAGANN | 956 | GGGCCCGCGAGAC | chr2:181684163- | 8 |
| 03.1 | | NNNNNGCTCGCGGG | | CGTGGGGCTCAGG | 181684193 | |
| | | CCC | | GGCCG | | |
| WP_0058241 | 350 | ACAAACCCTATANNN | 957 | ACATAGCCTATATCT | chr2:190037319- | 7 |
| 23.1 | | NNNNTATAGGGTTAC | | TCATTATAGGGTTA | 190037349 | |
| | | T | | TT | | |
| WP_0008178 | 351 | TACACGTTACATANN | 958 | TATACTTTACATACT | chr2:203639620- | 6 |
| 56.1 | | NNNNNNTATGTAAAT | | TTATTGTATGTAAAT | 203639653 | |
| | | TGTA | | TATA | | |
| WP_0152177 | 352 | CTACCCAAGAGANNN | 959 | CTACCCAAGAGATA | chr2:21047490- | 5 |
| 82.1 | | NNNNNNACTGTTGG | | AGGTCAGAATGTTG | 21047522 | |
| | | GTAG | | AGTCG | | |
| WP_0707260 | 353 | ATAAGTTATGATANN | 960 | ATAAGTAATGATAA | chr2:214027139- | 6 |
| 79.1 | | NNNNNNNTATCATAA | | AATATTAGTATGAT | 214027173 | |
| | | CCTAT | | AACCTTT | | |
| WP_0000596 | 354 | CTATTAGCCACANNN | 961 | CCAGTAGCCACAAG | chr2:217887121- | 6 |
| 22.1 | | NNNNNTGTAGCAAAT | | TGATAGTCTAGCAA | 217887152 | |
| | | AG | | ATAG | | |
| WP_0153698 | 355 | CTATTAGCCACANNN | 962 | CCAGTAGCCACAAG | chr2:217887121- | 6 |
| 06.1 | | NNNNNTGTAGCAAAT | | TGATAGTCTAGCAA | 217887152 | |
| | | AG | | ATAG | | |
| WP_0130588 | 356 | TCTGTAACAAGANNN | 963 | TCTGTAAGAAGAAG | chr2:223156070- | 8 |
| 85.1 | | NNNNNTCTTGTTACA | | GAACACACTTCTTA | 223156101 | |
| | | GA | | CAGA | | |
| WP_0130582 | 357 | TCTGTAACAAGANNN | 964 | TCTGTAAGAAGAAG | chr2:223156070- | 8 |
| 63.1 | | NNNNNTCTTGTTACA | | GAACACACTTCTTA | 223156101 | |
| | | GA | | CAGA | | |
| WP_0569221 | 358 | GGCGGCCCGACANN | 965 | GGCGGCCCGGCTTG | chr2:231037589- | 7 |
| 10.1 | | NNNNNNNTGCCGGG | | CGCGCCCTGCCGAG | 231037621 | |
| | | CCGCC | | CCGCC | | |
| WP_0544480 | 359 | AACAGCCGAAGANN | 966 | AACAGCCCAAGAAT | chr2:23112541- | 6 |
| 37.1 | | NNNNNNTCTTCGGCC | | TTGTGTTCCTCGGC | 23112572 | |
| | | TTT | | CATT | | |
| WP_0107446 | 360 | CCCTTGCAAAGANNN | 967 | CCCTTGCAAAGGCT | chr2:236703920- | 7 |
| 10.1 | | NNNNNNTCATTTCAA | | TCAACCATCATTTCA | 236703952 | |
| | | GGG | | GGTG | | |
| WP_0161799 | 361 | CCCTTGCAAAGANNN | 968 | CCCTTGCAAAGGCT | chr2:236703920- | 7 |
| 37.1 | | NNNNNNTCATTTCAA | | TCAACCATCATTTCA | 236703952 | |
| | | GGG | | GGTG | | |
| WP_0492204 | 362 | CCCTTGCAAAGANNN | 969 | CCCTTGCAAAGGCT | chr2:236703920- | 7 |
| 44.1 | | NNNNNNTCATTTCAA | | TCAACCATCATTTCA | 236703952 | |
| | | GGG | | GGTG | | |
| WP_0889323 | 363 | CCCTTGCAAAGANNN | 970 | CCCTTGCAAAGGCT | chr2:236703920- | 7 |
| 58.1 | | NNNNNNTCATTTCAA | | TCAACCATCATTTCA | 236703952 | |
| | | GGG | | GGTG | | |
| WP_0212680 | 364 | GACTGGCAAAGANN | 971 | GACTGAGAAAGAG | chr2:25905759- | 5 |
| 46.1 | | NNNNNGCTTTGTCAG | | AAAGCCACTTTGTC | 25905789 | |
| | | TC | | AGTC | | |
| WP_0515175 | 365 | AGCGGCGGGAGANN | 972 | GGCGGCGGGAGGT | chr2:29921526- | 5 |
| 28.1 | | NNNNNNGCTCCCACC | | ACCAGCTGCTACCA | 29921557 | |
| | | GCT | | CCGCT | | |
| WP_1002517 | 366 | CTACGTCTGATANNN | 973 | CTACGTCTGAGAAC | chr2:36563545- | 7 |
| 39.1 | | NNNNNNTATCAGAC | | GTGCTCCTATCAAA | 36563577 | |
| | | GCTG | | CGCTT | | |
| WP_0200945 | 367 | CTACGTCTGATANNN | 974 | CTACGTCTGAGAAC | chr2:36563545- | 7 |
| 36.1 | | NNNNNNTATCAGAC | | GTGCTCCTATCAAA | 36563577 | |
| | | GCTG | | CGCTT | | |
| WP_1039851 | 368 | CTACGTCTGATANNN | 975 | CTACGTCTGAGAAC | chr2:36563545- | 7 |
| 18.1 | | NNNNNNTATCAGAC | | GTGCTCCTATCAAA | 36563577 | |
| | | GCTG | | CGCTT | | |
| WP_0143509 | 369 | ATACCCCAGATANNN | 976 | ATATGCCAGATAAG | chr2:37280854- | 8 |
| 44.1 | | NNNNNNTATCCGGG | | GGACTAGTATCCAG | 37280886 | |
| | | GTAT | | GGTAT | | |
| WP_0245455 | 370 | AAGCTTACGATANNN | 977 | AAGCTTACCATAAT | chr2:50517209- | 6 |
| 67.1 | | NNNNNTTTCGTAAGC | | CTGATTTATGGTAA | 50517240 | |
| | | TT | | GCTT | | |
| WP_0226149 | 371 | GGTAGTAACAGANN | 978 | GGTAGCAACTGAAG | chr2:66826551- | 7 |
| 60.1 | | NNNNNACTGTTACTA | | GCTGGACTGTTTCT | 66826581 | |
| | | CC | | ACC | | |
| WP_0719741 | 372 | ACATGTCCGATANNN | 979 | ACATGTACAATAAA | chr2:88631593- | 6 |
| 81.1 | | NNNNNNTATTGGAC | | CTGAACCTATTGGA | 88631625 | |
| | | ATAT | | AATAT | | |
| WP_0095572 | 373 | TAGTTGGTGATANNN | 980 | TGGATGGTGATACA | chr2:94826665- | 7 |
| 65.1 | | NNNNNTATCACCAAC | | GATATTTATCATCAA | 94826696 | |
| | | TC | | CTC | | |
| WP_0698556 | 374 | GGGCCTGCGAGANN | 981 | GAGCCTGGGAGAA | chr20:33704755- | 6 |
| 69.1 | | NNNNNACTCGCAGG | | ATGCAGACTCTCAG | 33704785 | |
| | | CCC | | GCCC | | |
| WP_0854213 | 375 | AAACGACCGATANNN | 982 | AAATTACCGATAAT | chr20:34466535- | 6 |
| 89.1 | | NNNNNNTATCGTTCA | | ATTATTCTATCATTC | 34466567 | |
| | | TTT | | ATTT | | |
| WP_0624461 | 376 | TAGTGTCTGAGANNN | 983 | TAGTGTCTGTGTTT | chr21:15870374- | 6 |
| 29.1 | | NNNNNTCTCAGACAC | | ATTAGCTCTCAAAC | 15870405 | |
| | | TA | | ACTA | | |
| WP_0087262 | 377 | AGAACCCGGACANN | 984 | GGAACCCGGCCATC | chr22:23385516- | NA |
| 05.1 | | NNNNNNGTTCCGGG | | CCTCTGGTTCCTGG | 23385547 | |
| | | TTCT | | TTCT | | |
| WP_0545289 | 378 | AGGGTGTTGATANNN | 985 | AGGGTGTTGACAGC | chr22:32751606- | NA |
| 82.1 | | NNNNNNTATCACCAC | | AGTGGGATATCACC | 32751638 | |
| | | TCT | | ACCTT | | |
| KPL69881.1 | 379 | AGGGTGTTGATANNN | 986 | AGGGTGTTGACAGC | chr22:32751606- | NA |
| | | NNNNNNTATCACCAC | | AGTGGGATATCACC | 32751638 | |
| | | TCT | | ACCTT | | |
| SEM26217.1 | 380 | TTATGTCCGATANNN | 987 | TTAGGTCAGATACA | chr3:110856754- | 5 |
| | | NNNNNNTATTGGAC | | TTCCAAGTATTGGA | 110856786 | |
| | | ATAG | | AATAG | | |
| WP_1061655 | 381 | TTATGTCCGATANNN | 988 | TTAGGTCAGATACA | chr3:110856754- | 5 |
| 51.1 | | NNNNNNTATTGGAC | | TTCCAAGTATTGGA | 110856786 | |
| | | ATAG | | AATAG | | |
| WP_0083358 | 382 | TTATGTCCGATANNN | 989 | TTAGGTCAGATACA | chr3:110856754- | 5 |
| 38.1 | | NNNNNNTATTGGAC | | TTCCAAGTATTGGA | 110856786 | |
| | | ATAG | | AATAG | | |
| WP_0290696 | 383 | TTGGTTGGAATANNN | 990 | TTGGTGGGAATAAA | chr3:111817292- | 5 |
| 76.1 | | NNNNNNTATTCAAAC | | CAAACAGTATCCAA | 111817324 | |
| | | CAA | | ACCAC | | |
| WP_0118869 | 384 | GAATACAACATANNN | 991 | GAATACAACAAATA | chr3:117712816- | 6 |
| 69.1 | | NNNNNTATGTTGCAT | | TTTTTCTATGTAGCA | 117712847 | |
| | | TC | | TTT | | |
| WP_0478214 | 385 | AACTCGACAATANNN | 992 | AACTAGACAAGAAC | chr3:127281819- | 6 |
| 48.1 | | NNNNTAATGTCGAGT | | TTTAATAATGTCTAG | 127281849 | |
| | | T | | TT | | |
| WP_0478251 | 386 | AACTCGACAATANNN | 993 | AACTAGACAAGAAC | chr3:127281819- | 6 |
| 38.1 | | NNNNTAATGTCGAGT | | TTTAATAATGTCTAG | 127281849 | |
| | | T | | TT | | |
| WP_1165468 | 387 | ATTAACTTCATATANN | 994 | ATTAACCTCATATAT | chr3:150147140- | 5 |
| 38.1 | | NNNNNNTATATGAA | | GGGATCCAAAATGA | 150147175 | |
| | | GTTAAT | | AGTTAAT | | |
| WP_0869047 | 388 | TCTACCAGTGATANN | 995 | TCTTCCAGTGATAA | chr3:158595931- | 6 |
| 34.1 | | NNNNNNTATCACTGG | | AACCTAAAATCAGT | 158595964 | |
| | | TAGA | | GGTAGA | | |
| WP_1331810 | 389 | TCTACCAGTGATANN | 996 | TCTTCCAGTGATAA | chr3:158595931- | 6 |
| 36.1 | | NNNNNNTATCACTGG | | AACCTAAAATCAGT | 158595964 | |
| | | TAGA | | GGTAGA | | |
| WP_1092859 | 390 | TCTACCAGTGATANN | 997 | TCTTCCAGTGATAA | chr3:158595931- | 6 |
| 90.1 | | NNNNNNTATCACTGG | | AACCTAAAATCAGT | 158595964 | |
| | | TAGA | | GGTAGA | | |
| WP_1139404 | 391 | TCTACCAGTGATANN | 998 | TCTTCCAGTGATAA | chr3:158595931- | 6 |
| 03.1 | | NNNNNNTATCACTGG | | AACCTAAAATCAGT | 158595964 | |
| | | TAGA | | GGTAGA | | |
| ACK46586.1 | 392 | TCTACCAGTGATANN | 999 | TCTTCCAGTGATAA | chr3:158595931- | 6 |
| | | NNNNNNTATCACTGG | | AACCTAAAATCAGT | 158595964 | |
| | | TAGA | | GGTAGA | | |
| AEG11408.1 | 393 | TCTACCAGTGATANN | 1000 | TCTTCCAGTGATAA | chr3:158595931- | 6 |
| | | NNNNNNTATCACTGG | | AACCTAAAATCAGT | 158595964 | |
| | | TAGA | | GGTAGA | | |
| WP_0812484 | 394 | TCTACCAGTGATANN | 1001 | TCTTCCAGTGATAA | chr3:158595931- | 6 |
| 13.1 | | NNNNNNTATCACTGG | | AACCTAAAATCAGT | 158595964 | |
| | | TAGA | | GGTAGA | | |
| WP_0122771 | 395 | TCTACCAGTGATANN | 1002 | TCTTCCAGTGATAA | chr3:158595931- | 6 |
| 58.1 | | NNNNNNTATCACTGG | | AACCTAAAATCAGT | 158595964 | |
| | | TAGA | | GGTAGA | | |
| WP_0125868 | 396 | TCTACCAGTGATANN | 1003 | TCTTCCAGTGATAA | chr3:158595931- | 6 |
| 24.1 | | NNNNNNTATCACTGG | | AACCTAAAATCAGT | 158595964 | |
| | | TAGA | | GGTAGA | | |
| WP_0817290 | 397 | TCTACCAGTGATANN | 1004 | TCTTCCAGTGATAA | chr3:158595931- | 6 |
| 30.1 | | NNNNNNTATCACTGG | | AACCTAAAATCAGT | 158595964 | |
| | | TAGA | | GGTAGA | | |
| KZK70296.1 | 398 | TCTACCAGTGATANN | 1005 | TCTTCCAGTGATAA | chr3:158595931- | 6 |
| | | NNNNNNTATCACTGG | | AACCTAAAATCAGT | 158595964 | |
| | | TAGA | | GGTAGA | | |
| WP_0121545 | 399 | CTACCAGTGATANNN | 1006 | CTTCCAGTGATAAA | chr3:158595932- | 6 |
| 34.1 | | NNNNNTATCACTGGT | | ACCTAAAATCAGTG | 158595963 | |
| | | AG | | GTAG | | |
| ABV87414.1 | 400 | CTACCAGTGATANNN | 1007 | CTTCCAGTGATAAA | chr3:158595932- | 6 |
| | | NNNNNTATCACTGGT | | ACCTAAAATCAGTG | 158595963 | |
| | | AG | | GTAG | | |
| WP_0116227 | 401 | CTACCAGTGATANNN | 1008 | CTTCCAGTGATAAA | chr3:158595932- | 6 |
| 13.1 | | NNNNNTATCACTGGT | | ACCTAAAATCAGTG | 158595963 | |
| | | AG | | GTAG | | |
| WP_0517141 | 402 | CTACCAGTGATANNN | 1009 | CTTCCAGTGATAAA | chr3:158595932- | 6 |
| 41.1 | | NNNNNTATCACTGGT | | ACCTAAAATCAGTG | 158595963 | |
| | | AG | | GTAG | | |
| WP_0777514 | 403 | CTACCAGTGATANNN | 1010 | CTTCCAGTGATAAA | chr3:158595932- | 6 |
| 11.1 | | NNNNNTATCACTGGT | | ACCTAAAATCAGTG | 158595963 | |
| | | AG | | GTAG | | |
| WP_0130514 | 404 | CTACCAGTGATANNN | 1011 | CTTCCAGTGATAAA | chr3:158595932- | 6 |
| 10.1 | | NNNNNTATCACTGGT | | ACCTAAAATCAGTG | 158595963 | |
| | | AG | | GTAG | | |
| WP_1153345 | 405 | CTACCAGTGATANNN | 1012 | CTTCCAGTGATAAA | chr3:158595932- | 6 |
| 56.1 | | NNNNNTATCACTGGT | | ACCTAAAATCAGTG | 158595963 | |
| | | AG | | GTAG | | |
| WP_1264918 | 406 | CTACCAGTGATANNN | 1013 | CTTCCAGTGATAAA | chr3:158595932- | 6 |
| 84.1 | | NNNNNTATCACTGGT | | ACCTAAAATCAGTG | 158595963 | |
| | | AG | | GTAG | | |
| WP_0209126 | 407 | CTACCAGTGATANNN | 1014 | CTTCCAGTGATAAA | chr3:158595932- | 6 |
| 17.1 | | NNNNNTATCACTGGT | | ACCTAAAATCAGTG | 158595963 | |
| | | AG | | GTAG | | |
| WP_0882111 | 408 | CTACCAGTGATANNN | 1015 | CTTCCAGTGATAAA | chr3:158595932- | 6 |
| 52.1 | | NNNNNTATCACTGGT | | ACCTAAAATCAGTG | 158595963 | |
| | | AG | | GTAG | | |
| WP_0116261 | 409 | CTACCAGTGATANNN | 1016 | CTTCCAGTGATAAA | chr3:158595932- | 6 |
| 97.1 | | NNNNNTATCACTGGT | | ACCTAAAATCAGTG | 158595963 | |
| | | AG | | GTAG | | |
| WP_0110723 | 410 | CTACCAGTGATANNN | 1017 | CTTCCAGTGATAAA | chr3:158595932- | 6 |
| 65.1 | | NNNNNTATCACTGGT | | ACCTAAAATCAGTG | 158595963 | |
| | | AG | | GTAG | | |
| WP_0694554 | 411 | CTACCAGTGATANNN | 1018 | CTTCCAGTGATAAA | chr3:158595932- | 6 |
| 45.1 | | NNNNNTATCACTGGT | | ACCTAAAATCAGTG | 158595963 | |
| | | AG | | GTAG | | |
| WP_0509913 | 412 | CTACCAGTGATANNN | 1019 | CTTCCAGTGATAAA | chr3:158595932- | 6 |
| 48.1 | | NNNNNTATCACTGGT | | ACCTAAAATCAGTG | 158595963 | |
| | | AG | | GTAG | | |
| WP_0556473 | 413 | CTACCAGTGATANNN | 1020 | CTTCCAGTGATAAA | chr3:158595932- | 6 |
| 63.1 | | NNNNNTATCACTGGT | | ACCTAAAATCAGTG | 158595963 | |
| | | AG | | GTAG | | |
| WP_1123527 | 414 | CTACCAGTGATANNN | 1021 | CTTCCAGTGATAAA | chr3:158595932- | 6 |
| 96.1 | | NNNNNTATCACTGGT | | ACCTAAAATCAGTG | 158595963 | |
| | | AG | | GTAG | | |
| WP_1052525 | 415 | CTACCAGTGATANNN | 1022 | CTTCCAGTGATAAA | chr3:158595932- | 6 |
| 41.1 | | NNNNNTATCACTGGT | | ACCTAAAATCAGTG | 158595963 | |
| | | AG | | GTAG | | |
| WP_0120892 | 416 | CTACCAGTGATANNN | 1023 | CTTCCAGTGATAAA | chr3:158595932- | 6 |
| 73.1 | | NNNNNTATCACTGGT | | ACCTAAAATCAGTG | 158595963 | |
| | | AG | | GTAG | | |
| WP_0719394 | 417 | CTACCAGTGATANNN | 1024 | CTTCCAGTGATAAA | chr3:158595932- | 6 |
| 73.1 | | NNNNNTATCACTGGT | | ACCTAAAATCAGTG | 158595963 | |
| | | AG | | GTAG | | |
| WP_0143580 | 418 | CTACCAGTGATANNN | 1025 | CTTCCAGTGATAAA | chr3:158595932- | 6 |
| 05.1 | | NNNNNTATCACTGGT | | ACCTAAAATCAGTG | 158595963 | |
| | | AG | | GTAG | | |
| WP_1066505 | 419 | CTACCAGTGATANNN | 1026 | CTTCCAGTGATAAA | chr3:158595932- | 6 |
| 61.1 | | NNNNNTATCACTGGT | | ACCTAAAATCAGTG | 158595963 | |
| | | AG | | GTAG | | |
| WP_0764115 | 420 | CTACCAGTGATANNN | 1027 | CTTCCAGTGATAAA | chr3:158595932- | 6 |
| 19.1 | | NNNNNTATCACTGGT | | ACCTAAAATCAGTG | 158595963 | |
| | | AG | | GTAG | | |
| WP_0123250 | 421 | CTACCAGTGATANNN | 1028 | CTTCCAGTGATAAA | chr3:158595932- | 6 |
| 03.1 | | NNNNNTATCACTGGT | | ACCTAAAATCAGTG | 158595963 | |
| | | AG | | GTAG | | |
| WP_1010902 | 422 | CTACCAGTGATANNN | 1029 | CTTCCAGTGATAAA | chr3:158595932- | 6 |
| 09.1 | | NNNNNTATCACTGGT | | ACCTAAAATCAGTG | 158595963 | |
| | | AG | | GTAG | | |
| WP_1151369 | 423 | CTACCAGTGATANNN | 1030 | CTTCCAGTGATAAA | chr3:158595932- | 6 |
| 67.1 | | NNNNNTATCACTGGT | | ACCTAAAATCAGTG | 158595963 | |
| | | AG | | GTAG | | |
| WP_0647913 | 424 | CTACCAGTGATANNN | 1031 | CTTCCAGTGATAAA | chr3:158595932- | 6 |
| 49.1 | | NNNNNTATCACTGGT | | ACCTAAAATCAGTG | 158595963 | |
| | | AG | | GTAG | | |
| WP_0121425 | 425 | CTACCAGTGATANNN | 1032 | CTTCCAGTGATAAA | chr3:158595932- | 6 |
| 88.1 | | NNNNNTATCACTGGT | | ACCTAAAATCAGTG | 158595963 | |
| | | AG | | GTAG | | |
| WP_1265205 | 426 | CTACCAGTGATANNN | 1033 | CTTCCAGTGATAAA | chr3:158595932- | 6 |
| 63.1 | | NNNNNTATCACTGGT | | ACCTAAAATCAGTG | 158595963 | |
| | | AG | | GTAG | | |
| WP_1089465 | 427 | CTACCAGTGATANNN | 1034 | CTTCCAGTGATAAA | chr3:158595932- | 6 |
| 65.1 | | NNNNNTATCACTGGT | | ACCTAAAATCAGTG | 158595963 | |
| | | AG | | GTAG | | |
| WP_0374112 | 428 | CTACCAGTGATANNN | 1035 | CTTCCAGTGATAAA | chr3:158595932- | 6 |
| 15.1 | | NNNNNTATCACTGGT | | ACCTAAAATCAGTG | 158595963 | |
| | | AG | | GTAG | | |
| OIO40422.1 | 429 | CCGTACTATATANNN | 1036 | CGCTACTATATAAA | chr3:162275981- | 5 |
| | | NNNNNTATATAATGC | | GAGTAATATATAAT | 162276012 | |
| | | GG | | GCAG | | |
| WP_0479148 | 430 | GAAACGTTGATANNN | 1037 | GAAATGTTCATAAT | chr3:164474658- | 5 |
| 82.1 | | NNNNNNTATTAACGT | | ATTCCTTTATTAATG | 164474690 | |
| | | TTT | | TTTT | | |
| WP_0107292 | 431 | GAAACGTTGATANNN | 1038 | GAAATGTTCATAAT | chr3:164474658- | 5 |
| 68.1 | | NNNNNNTATTAACGT | | ATTCCTTTATTAATG | 164474690 | |
| | | TTT | | TTTT | | |
| WP_0031719 | 432 | AAACCCTCAACANNN | 1039 | AAACCCTCAACAAA | chr3:166919839- | 7 |
| 84.1 | | NNNNTGTCAAGGGTT | | CTAAGTATCAAAGG | 166919869 | |
| | | T | | TAT | | |
| WP_0336601 | 433 | AAACCCTCAACANNN | 1040 | AAACCCTCAACAAA | chr3:166919839- | 7 |
| 84.1 | | NNNNTGTCAAGGGTT | | CTAAGTATCAAAGG | 166919869 | |
| | | T | | TAT | | |
| WP_0020768 | 434 | AAACCCTCAACANNN | 1041 | AAACCCTCAACAAA | chr3:166919839- | 7 |
| 80.1 | | NNNNTGTCAAGGGTT | | CTAAGTATCAAAGG | 166919869 | |
| | | T | | TAT | | |
| WP_0161158 | 435 | AAACCCTCAACANNN | 1042 | AAACCCTCAACAAA | chr3:166919839- | 7 |
| 18.1 | | NNNNTGTCAAGGGTT | | CTAAGTATCAAAGG | 166919869 | |
| | | T | | TAT | | |
| WP_0117361 | 436 | TCGGTATATATANNN | 1043 | TCTGTATATATAAG | chr3:174585052- | 5 |
| 63.1 | | NNNNCACATATACCG | | AATAACACATATTCT | 174585082 | |
| | | A | | GA | | |
| WP_0444023 | 437 | CATCAAGTGATANNN | 1044 | CTTCAAGTGATATT | chr3:27705115- | 5 |
| 40.1 | | NNNNNTATCGCTTGA | | ATATTATACCACTTG | 27705146 | |
| | | TG | | ATG | | |
| WP_0084001 | 438 | GCAGAGTGAAGANN | 1045 | TCAGAGGGAAGAA | chr3:48141565- | 5 |
| 48.1 | | NNNNNNTCCTCGCTC | | TACCTGCTCCTGGC | 48141596 | |
| | | TGC | | TCTGC | | |
| WP_0568715 | 439 | AAAAACGGCATANNN | 1046 | AAAAATGGTATAAG | chr3:50885338- | 6 |
| 37.1 | | NNNNNTATGCCGTTT | | CTTTTGTATGCAGTT | 50885369 | |
| | | TT | | TTT | | |
| WP_0029908 | 440 | TTAATGAGTAGANNN | 1047 | TTAATGAGTACACA | chr3:54189864- | 6 |
| 81.1 | | NNNNNTCTACTCATT | | TAATTTTLTALTTTT | 54189895 | |
| | | AA | | TAA | | |
| WP_0418906 | 441 | TTAATGAGTAGANNN | 1048 | TTAATGAGTACACA | chr3:54189864- | 6 |
| 31.1 | | NNNNNTCTACTCATT | | TAATTTTLTALTTTT | 54189895 | |
| | | AA | | TAA | | |
| WP_0112793 | 442 | AGGTTAATATAGANN | 1049 | AGGTTAAAATAGAC | chr3:60883844- | 4 |
| 65.1 | | NNNNNNTTTATATTA | | AAATGGGATTATAT | 60883877 | |
| | | AGCT | | CAAGCT | | |
| YP_0092216 | 443 | ATAAGACATAGANNN | 1050 | ATAAGCCATAGAGC | chr3:64770759- | 6 |
| 49.1 | | NNNNNNTCTATGTCT | | CCCCATCTCTGTGTC | 64770791 | |
| | | TAT | | CTAT | | |
| WP_0763847 | 444 | CTGGCAAGCCATANN | 1051 | CTGGCAAGGCATAA | chr3:86065715- | 5 |
| 67.1 | | NNNNNNNTATATCTT | | AGGTACGTTATATT | 86065749 | |
| | | GCCAG | | TAGCCAG | | |
| WP_0171356 | 445 | CTGGCAAGCCATANN | 1052 | CTGGCAAGGCATAA | chr3:86065715- | 5 |
| 69.1 | | NNNNNNNTATATCTT | | AGGTACGTTATATT | 86065749 | |
| | | GCCAG | | TAGCCAG | | |
| WP_1026053 | 446 | TGACCCACGATANNN | 1053 | TGAACCACAATATT | chr3:95971700- | 5 |
| 25.1 | | NNNNNNTATCGTGG | | TCTCAACTATCTTGG | 95971732 | |
| | | GTGA | | GTGA | | |
| WP_0028277 | 447 | GAAGTTGGGACANN | 1054 | CAGGTTGGGACCAT | chr4:108054576- | 5 |
| 82.1 | | NNNNNNTGTTCCAAC | | TTCTGCTGTTCCAAC | 108054607 | |
| | | TTC | | TTC | | |
| WP_0695521 | 448 | TTAGGTCTGATANNN | 1055 | CTAGGTCTGATATC | chr4:143442555- | 5 |
| 41.1 | | NNNNNNTATCCGACA | | ACTCATGTATCCCAC | 143442587 | |
| | | TAA | | ATTA | | |
| AZE17458.1 | 449 | TTAGGTCTGATANNN | 1056 | CTAGGTCTGATATC | chr4:143442555- | 5 |
| | | NNNNNNTATCCGACC | | ACTCATGTATCCCAC | 143442587 | |
| | | TTA | | ATTA | | |
| SDY43398.1 | 450 | TTAGGTCTGATANNN | 1057 | CTAGGTCTGATATC | chr4:143442555- | 5 |
| | | NNNNNNTATCCGACC | | ACTCATGTATCCCAC | 143442587 | |
| | | TTA | | ATTA | | |
| AZD92641.1 | 451 | TTAGGTCTGATANNN | 1058 | CTAGGTCTGATATC | chr4:143442555- | 5 |
| | | NNNNNNTATCCGACC | | ACTCATGTATCCCAC | 143442587 | |
| | | TTA | | ATTA | | |
| WP_0821432 | 452 | TTAGGTCTGATANNN | 1059 | CTAGGTCTGATATC | chr4:143442555- | 5 |
| 26.1 | | NNNNNNTATCCGACC | | ACTCATGTATCCCAC | 143442587 | |
| | | TTA | | ATTA | | |
| WP_1106236 | 453 | TTAGGTCTGATANNN | 1060 | CTAGGTCTGATATC | chr4:143442555- | 5 |
| 42.1 | | NNNNNNTATCCGACC | | ACTCATGTATCCCAC | 143442587 | |
| | | TTA | | ATTA | | |
| RIA35947.1 | 454 | TTAGGTCTGATANNN | 1061 | CTAGGTCTGATATC | chr4:143442555- | 5 |
| | | NNNNNNTATCCGACC | | ACTCATGTATCCCAC | 143442587 | |
| | | TTA | | ATTA | | |
| AZC51718.1 | 455 | TTAGGTCTGATANNN | 1062 | CTAGGTCTGATATC | chr4:143442555- | 5 |
| | | NNNNNNTATCCGACC | | ACTCATGTATCCCAC | 143442587 | |
| | | TTA | | ATTA | | |
| WP_0034523 | 456 | TTAGGTCTGATANNN | 1063 | CTAGGTCTGATATC | chr4:143442555- | 5 |
| 52.1 | | NNNNNNTATCCGACC | | ACTCATGTATCCCAC | 143442587 | |
| | | TTA | | ATTA | | |
| WP_1080997 | 457 | TTAGGTCTGATANNN | 1064 | CTAGGTCTGATATC | chr4:143442555- | 5 |
| 39.1 | | NNNNNNTATCCGACC | | ACTCATGTATCCCAC | 143442587 | |
| | | TTA | | ATTA | | |
| WP_1106375 | 458 | TTAGGTCTGATANNN | 1065 | CTAGGTCTGATATC | chr4:143442555- | 5 |
| 60.1 | | NNNNNNTATCCGACC | | ACTCATGTATCCCAC | 143442587 | |
| | | TTA | | ATTA | | |
| WP_0452178 | 459 | TTAGGTCTGATANNN | 1066 | CTAGGTCTGATATC | chr4:143442555- | 5 |
| 96.1 | | NNNNNNTATCCGACC | | ACTCATGTATCCCAC | 143442587 | |
| | | TTA | | ATTA | | |
| WP_1283253 | 460 | TTAGGTCTGATANNN | 1067 | CTAGGTCTGATATC | chr4:143442555- | 5 |
| 17.1 | | NNNNNNTATCCGACC | | ACTCATGTATCCCAC | 143442587 | |
| | | TTA | | ATTA | | |
| OWK92550.1 | 461 | TTAGGTCTGATANNN | 1068 | CTAGGTCTGATATC | chr4:143442555- | 5 |
| | | NNNNNNTATCCGACC | | ACTCATGTATCCCAC | 143442587 | |
| | | TTA | | ATTA | | |
| WP_0247174 | 462 | TTAGGTCTGATANNN | 1069 | CTAGGTCTGATATC | chr4:143442555- | 5 |
| 80.1 | | NNNNNNTATCCGACC | | ACTCATGTATCCCAC | 143442587 | |
| | | TTA | | ATTA | | |
| WP_1012936 | 463 | TTAGGTCTGATANNN | 1070 | CTAGGTCTGATATC | chr4:143442555- | 5 |
| 15.1 | | NNNNNNTATCCGACC | | ACTCATGTATCCCAC | 143442587 | |
| | | TTA | | ATTA | | |
| WP_0316426 | 464 | TTAGGTCTGATANNN | 1071 | CTAGGTCTGATATC | chr4:143442555- | 5 |
| 20.1 | | NNNNNNTATCCGACC | | ACTCATGTATCCCAC | 143442587 | |
| | | TTA | | ATTA | | |
| WP_0429487 | 465 | TTAGGTCTGATANNN | 1072 | CTAGGTCTGATATC | chr4:143442555- | 5 |
| 96.1 | | NNNNNNTATCCGACC | | ACTCATGTATCCCAC | 143442587 | |
| | | TTA | | ATTA | | |
| WP_1033260 | 466 | TGACAGTGGATANNN | 1073 | TGAAAGTGGAGAA | chr4:160047452- | 4 |
| 70.1 | | NNNNNNTATCCAATC | | ATAAGAACAATCCA | 160047484 | |
| | | TCA | | ATCTCA | | |
| WP_0764496 | 467 | TTAGTTATGATANNN | 1074 | TTAGTTATTATAACT | chr4:172157070- | 7 |
| 57.1 | | NNNNGATCATAACTA | | TTCCTATTATAACTA | 172157100 | |
| | | A | | A | | |
| WP_0746356 | 468 | GCTATCTGAACANNN | 1075 | GCTATATGAACAGA | chr4:176324510- | 4 |
| 93.1 | | NNNNNTGTTCAGATT | | CGTTAATGTTCATAT | 176324541 | |
| | | GA | | TCA | | |
| WP_0346339 | 469 | GATGACTTTACANNN | 1076 | GATGACTTTACCCT | chr4:187632588- | 5 |
| 66.1 | | NNNNNTGTAAAGTCA | | ATTTCTTGTGAAGT | 187632619 | |
| | | TC | | GATC | | |
| WP_0125492 | 470 | CTCAATTTCACANNN | 1077 | CTCAATTACACACCT | chr4:46313749- | 6 |
| 23.1 | | NNNNTGTGAAATTGA | | GAGATTTGAAATTC | 46313779 | |
| | | G | | AG | | |
| WP_0161104 | 471 | AAGGGGAACAGANN | 1078 | AAGAGGAACAGAT | chr4:74631209- | 6 |
| 51.1 | | NNNNNTCCGTTCCCC | | ATTCTTTCCCTTCCC | 74631239 | |
| | | TT | | ATT | | |
| WP_0486588 | 472 | AGCTAGGTAAGANN | 1079 | AGATAGGTAAGATT | chr4:76517527- | 6 |
| 60.1 | | NNNNNNTCTTACCTA | | TAGGATTCTTATCCA | 76517558 | |
| | | TGT | | TGT | | |
| WP_0699453 | 473 | GAAATCGTAATANNN | 1080 | GAAATATTAATAAC | chr4:80833020- | 5 |
| 92.1 | | NNNNNTATTACGATT | | TGAAAGTATTACGT | 80833051 | |
| | | TG | | TTTG | | |
| WP_0850707 | 474 | TATTACTATTGATANN | 1081 | TATAACTAGTGATA | chr5:110266292- | 5 |
| 31.1 | | NNNNNNNTATCACTA | | GATAACAGTTATCA | 110266328 | |
| | | GTAATA | | CTAGTTATA | | |
| OCW82643.1 | 475 | ATTACTATTGATANN | 1082 | ATAACTAGTGATAG | chr5:110266293- | 5 |
| | | NNNNNNNTATCACTA | | ATAACAGTTATCAC | 110266327 | |
| | | GTAAT | | TAGTTAT | | |
| WP_0374128 | 476 | ACTGAGCTAATANNN | 1083 | ACTGAATAAATATT | chr5:112739101- | 5 |
| 68.1 | | NNNNTATTAATTCAG | | TAAGATATTAATTC | 112739131 | |
| | | T | | AGT | | |
| WP_0765913 | 477 | ATCACACAGGATANN | 1084 | AACAAACAGGATAT | chr5:114709938- | 5 |
| 09.1 | | NNNNNNNTATCCTGT | | AAAGTGGTAATCCT | 114709972 | |
| | | TTTAT | | GTTTTAT | | |
| WP_0135253 | 478 | TAACGAACGATANNN | 1085 | TAACTAACGATACT | chr5:125436112- | 6 |
| 33.1 | | NNNNNNTATCATTCG | | TCTCAGATATAATTC | 125436144 | |
| | | TTG | | CTTG | | |
| WP_1274026 | 479 | TAACGAACGATANNN | 1086 | TAACTAACGATACT | chr5:125436112- | 6 |
| 74.1 | | NNNNNNTATCATTCG | | TCTCAGATATAATTC | 125436144 | |
| | | TTG | | CTTG | | |
| WP_0666056 | 480 | AGAATGGGCAGANN | 1087 | AGAATGGGCAGAA | chr5:129423741- | 5 |
| 81.1 | | NNNNNNTCTGACCCT | | AGAATGTTCTGGGA | 129423772 | |
| | | TCT | | CTTCT | | |
| WP_0809570 | 481 | TAGCTCTGGAGANNN | 1088 | TAGCTCTGGAGATA | chr5:13238067- | 7 |
| 39.1 | | NNNNNNTCTCCGGA | | GAGAGGCCCTTCAG | 13238099 | |
| | | GTTA | | AGTTA | | |
| KKX62373.1 | 482 | TAGCTCTGGAGANNN | 1089 | TAGCTCTGGAGATA | chr5:13238067- | 7 |
| | | NNNNNNTCTCCGGA | | GAGAGGCCCTTCAG | 13238099 | |
| | | GTTA | | AGTTA | | |
| WP_0400411 | 483 | AAGGGCTACAGANN | 1090 | GAGGGCTGAAGAC | chr5:13815922- | 7 |
| 54.1 | | NNNNNTCTGTAACCC | | AGAGGCTCTGTAAC | 13815952 | |
| | | TT | | CCTT | | |
| WP_0046914 | 484 | TGTTTGTTGATANNN | 1091 | TCTTTGTTGATAAGT | chr5:156255946- | 6 |
| 81.1 | | NNNNTATGGACAAAC | | ATTTTTTGTACAAAC | 156255976 | |
| | | A | | A | | |
| WP_0490066 | 485 | CCAGCGCTCAGANNN | 1092 | CCAGAGCACAGAG | chr5:168937193- | 6 |
| 36.1 | | NNNNNGCTGAGTGC | | GCCAAGGGGTGAG | 168937224 | |
| | | TGG | | TGCTGG | | |
| WP_1044604 | 486 | CCAGCGCTCAGANNN | 1093 | CCAGAGCACAGAG | chr5:168937193- | 6 |
| 35.1 | | NNNNNGCTGAGTGC | | GCCAAGGGGTGAG | 168937224 | |
| | | TGG | | TGCTGG | | |
| WP_0041869 | 487 | CCAGCGCTCAGANNN | 1094 | CCAGAGCACAGAG | chr5:168937193- | 6 |
| 33.1 | | NNNNNGCTGAGTGC | | GCCAAGGGGTGAG | 168937224 | |
| | | TGG | | TGCTGG | | |
| WP_0943201 | 488 | CCAGCGCTCAGANNN | 1095 | CCAGAGCACAGAG | chr5:168937193- | 6 |
| 39.1 | | NNNNNGCTGAGTGC | | GCCAAGGGGTGAG | 168937224 | |
| | | TGG | | TGCTGG | | |
| WP_0324356 | 489 | CCAGCGCTCAGANNN | 1096 | CCAGAGCACAGAG | chr5:168937193- | 6 |
| 50.1 | | NNNNNGCTGAGTGC | | GCCAAGGGGTGAG | 168937224 | |
| | | TGG | | TGCTGG | | |
| WP_0143865 | 490 | CCAGCGCTCAGANNN | 1097 | CCAGAGCACAGAG | chr5:168937193- | 6 |
| 29.1 | | NNNNNGCTGAGTGC | | GCCAAGGGGTGAG | 168937224 | |
| | | TGG | | TGCTGG | | |
| WP_0179011 | 491 | CCAGCGCTCAGANNN | 1098 | CCAGAGCACAGAG | chr5:168937193- | 6 |
| 02.1 | | NNNNNGCTGAGTGC | | GCCAAGGGGTGAG | 168937224 | |
| | | TGG | | TGCTGG | | |
| WP_1102048 | 492 | CCAGCGCTCAGANNN | 1099 | CCAGAGCACAGAG | chr5:168937193- | 6 |
| 72.1 | | NNNNNGCTGAGTGC | | GCCAAGGGGTGAG | 168937224 | |
| | | TGG | | TGCTGG | | |
| WP_0041975 | 493 | CCAGCGCTCAGANNN | 1100 | CCAGAGCACAGAG | chr5:168937193- | 6 |
| 71.1 | | NNNNNGCTGAGTGC | | GCCAAGGGGTGAG | 168937224 | |
| | | TGG | | TGCTGG | | |
| WP_0877285 | 494 | CCAGCGCTCAGANNN | 1101 | CCAGAGCACAGAG | chr5:168937193- | 6 |
| 82.1 | | NNNNNGCTGAGTGC | | GCCAAGGGGTGAG | 168937224 | |
| | | TGG | | TGCTGG | | |
| WP_0324132 | 495 | CCAGCGCTCAGANNN | 1102 | CCAGAGCACAGAG | chr5:168937193- | 6 |
| 33.1 | | NNNNNGCTGAGTGC | | GCCAAGGGGTGAG | 168937224 | |
| | | TGG | | TGCTGG | | |
| WP_0969037 | 496 | CCAGCGCTCAGANNN | 1103 | CCAGAGCACAGAG | chr5:168937193- | 6 |
| 42.1 | | NNNNNGCTGAGTGC | | GCCAAGGGGTGAG | 168937224 | |
| | | TGG | | TGCTGG | | |
| WP_1309532 | 497 | CCAGCGCTCAGANNN | 1104 | CCAGAGCACAGAG | chr5:168937193- | 6 |
| 38.1 | | NNNNNGCTGAGTGC | | GCCAAGGGGTGAG | 168937224 | |
| | | TGG | | TGCTGG | | |
| VGI65087.1 | 498 | CCAGCGCTCAGANNN | 1105 | CCAGAGCACAGAG | chr5:168937193- | 6 |
| | | NNNNNGCTGAGTGC | | GCCAAGGGGTGAG | 168937224 | |
| | | TGG | | TGCTGG | | |
| WP_0853533 | 499 | CCAGCGCTCAGANNN | 1106 | CCAGAGCACAGAG | chr5:168937193- | 6 |
| 66.1 | | NNNNNGCTGAGTGC | | GCCAAGGGGTGAG | 168937224 | |
| | | TGG | | TGCTGG | | |
| WP_0809229 | 500 | CCAGCGCTCAGANNN | 1107 | CCAGAGCACAGAG | chr5:168937193- | 6 |
| 91.1 | | NNNNNGCTGAGTGC | | GCCAAGGGGTGAG | 168937224 | |
| | | TGG | | TGCTGG | | |
| WP_1157936 | 501 | CCAGCGCTCAGANNN | 1108 | CCAGAGCACAGAG | chr5:168937193- | 6 |
| 42.1 | | NNNNNGCTGAGTGC | | GCCAAGGGGTGAG | 168937224 | |
| | | TGG | | TGCTGG | | |
| WP_0853544 | 502 | CCAGCGCTCAGANNN | 1109 | CCAGAGCACAGAG | chr5:168937193- | 6 |
| 69.1 | | NNNNNGCTGAGTGC | | GCCAAGGGGTGAG | 168937224 | |
| | | TGG | | TGCTGG | | |
| WP_1261239 | 503 | CCAGCGCTCAGANNN | 1110 | CCAGAGCACAGAG | chr5:168937193- | 6 |
| 82.1 | | NNNNNGCTGAGTGC | | GCCAAGGGGTGAG | 168937224 | |
| | | TGG | | TGCTGG | | |
| WP_1079476 | 504 | TTACCAGTGATANNN | 1111 | TTACCAGTGAAAGA | chr5:17974903- | 6 |
| 08.1 | | NNNNNTATCACTGGT | | AGATAATAAAACTG | 17974934 | |
| | | AG | | GTAG | | |
| WP_0839159 | 505 | GTACAGGTGATANNN | 1112 | GTACAGGTGATACA | chr5:21040341- | 6 |
| 96.1 | | NNNNNNTATCACCTG | | TACTGGATATCCCC | 21040373 | |
| | | TTG | | TGATA | | |
| YP_0038569 | 506 | GCCCTGGTCAGANNN | 1113 | GCCGTGGCCAGAGT | chr5:38448769- | 6 |
| 19.1 | | NNNNNNTCTGACCG | | GTGCAGCTCTGACC | 38448801 | |
| | | GGGC | | TGGGC | | |
| WP_1329781 | 507 | TAACATGGGATANNN | 1114 | TAAAATATGATACC | chr5:45155486- | 5 |
| 17.1 | | NNNNNNTATCCCATG | | TTCAGTGTATCCCAT | 45155518 | |
| | | TTA | | GTTA | | |
| WP_0482200 | 508 | CTTACGAATAGANNN | 1115 | CTTACGAATAAACA | chr5:45667699- | 5 |
| 40.1 | | NNNNAATATTCGTAA | | CAACTAACATTAGT | 45667729 | |
| | | G | | AAG | | |
| WP_0023515 | 509 | AACGGCAAAATANNN | 1116 | AATGGCAAAATAAA | chr5:56153488- | 6 |
| 52.1 | | NNNNNTATTTTGACG | | TGGGGGTATTTTGA | 56153519 | |
| | | TT | | TATT | | |
| ORE41776.1 | 510 | TGAGCACTGATANNN | 1117 | TGAGCACTAATCCC | chr5:68330222- | 8 |
| | | NNNNNNTATCAGTGC | | AAAATCTTATCATTG | 68330254 | |
| | | TTA | | CTTA | | |
| WP_0127298 | 511 | AAGCCCGGTAGANN | 1118 | TAGCCCGGTAGAGG | chr5:81344667- | 6 |
| 69.1 | | NNNNNTCTACCGGGC | | TGAGGTCTTCAGGG | 81344697 | |
| | | TT | | CTT | | |
| WP_1034222 | 512 | CAAGTATCGATANNN | 1119 | TAAGTATCTATATTT | chr6:120945709- | 5 |
| 07.1 | | NNNNNTATCGATATT | | CTATATATAGATATT | 120945740 | |
| | | TA | | TA | | |
| WP_0857349 | 513 | CAAGTATCGATANNN | 1120 | TAAGTATCTATATTT | chr6:120945709- | 5 |
| 74.1 | | NNNNNTATCGATATT | | CTATATATAGATATT | 120945740 | |
| | | TA | | TA | | |
| WP_0486675 | 514 | ATAGTGTGATATANN | 1121 | ATAGTGTAATATAA | chr6:126292077- | 6 |
| 03.1 | | NNNNNNTATATCACA | | TATAAATTATATAAC | 126292110 | |
| | | TTAT | | AATAT | | |
| WP_0764996 | 515 | GGCTTAGCTATANNN | 1122 | GGCTTAGCAATAAA | chr6:130946245- | 6 |
| 65.1 | | NNNNNGTTAGCTAA | | CCTATTGTTACATAA | 130946276 | |
| | | GCC | | GCC | | |
| WP_0458292 | 516 | TAATAGCGAATANNN | 1123 | TAATAGTGAATATG | chr6:133420190- | 6 |
| 69.1 | | NNNNNTATTCGCTAT | | CATTCATATTCACTA | 133420221 | |
| | | TG | | TTA | | |
| KJV34819.1 | 517 | TAATAGCGAATANNN | 1124 | TAATAGTGAATATG | chr6:133420190- | 6 |
| | | NNNNNTATTCGCTAT | | CATTCATATTCACTA | 133420221 | |
| | | TG | | TTA | | |
| WP_0732857 | 518 | TAAGGTATGATANNN | 1125 | GAAGATATTATATT | chr6:134634933- | 4 |
| 21.1 | | NNNNNNTATCATACC | | ATCTGTATATCATAC | 134634965 | |
| | | TTA | | CTTA | | |
| WP_1254233 | 519 | TAAGGTATGATANNN | 1126 | GAAGATATTATATT | chr6:134634933- | 4 |
| 73.1 | | NNNNNNTATCATACC | | ATCTGTATATCATAC | 134634965 | |
| | | TTA | | CTTA | | |
| WP_0355601 | 520 | TAAGGTATGATANNN | 1127 | GAAGATATTATATT | chr6:134634933- | 4 |
| 63.1 | | NNNNNNTATCATACC | | ATCTGTATATCATAC | 134634965 | |
| | | TTA | | CTTA | | |
| WP_1114806 | 521 | TAAGGTATGATANNN | 1128 | GAAGATATTATATT | chr6:134634933- | 4 |
| 23.1 | | NNNNNNTATCATACC | | ATCTGTATATCATAC | 134634965 | |
| | | TTA | | CTTA | | |
| WP_1254406 | 522 | TAAGGTATGATANNN | 1129 | GAAGATATTATATT | chr6:134634933- | 4 |
| 09.1 | | NNNNNNTATCATACC | | ATCTGTATATCATAC | 134634965 | |
| | | TTA | | CTTA | | |
| WP_0652356 | 523 | TTGGGATAGATANNN | 1130 | CTGAGATATATATA | chr6:146027378- | 4 |
| 45.1 | | NNNNNTATCTACCCC | | CAAAGATATCTACC | 146027409 | |
| | | AA | | CCAA | | |
| WP_0094081 | 524 | AGAGAGTAGATANN | 1131 | AGAGAGTATATATA | chr6:152603807- | 6 |
| 53.1 | | NNNNNNGATCTACTC | | TATATAGATATACT | 152603838 | |
| | | TCT | | ATCT | | |
| WP_1332888 | 525 | TAACACACCATANNN | 1132 | AAACACACCATATT | chr6:152964488- | 7 |
| 65.1 | | NNNNNNTATAGCGT | | CCCTTCATAGAGCG | 152964520 | |
| | | GTTA | | TATTA | | |
| WP_0114150 | 526 | AGACATGTGATANNN | 1133 | GGACAAGTGTTATT | chr6:153314283- | 6 |
| 80.1 | | NNNNNNTATCACATG | | TAATTCCTATCACAT | 153314315 | |
| | | TTG | | GTTG | | |
| YP_239821.1 | 527 | TATCCCTTGATANNN | 1134 | AATCCCTTGAAATT | chr6:22061867- | 4 |
| | | NNNNNNTTTCAAGG | | GTCAGTATTTCAAG | 22061899 | |
| | | GGTA | | GGTTA | | |
| WP_0186216 | 528 | TTATCTACGATANNN | 1135 | TTATCTAGGATAGG | chr6:25581730- | 5 |
| 39.1 | | NNNNNNTATCGTAG | | AAATCCTTATTCTAG | 25581762 | |
| | | ATAA | | ATAA | | |
| WP_0262423 | 529 | CTATGTCCGATANNN | 1136 | CTATGTCCGATTTCT | chr6:30376959- | 5 |
| 20.1 | | NNNNNNTATCGGAC | | TCTCATTATTGGACT | 30376991 | |
| | | ATAA | | TAA | | |
| AVC45611.1 | 530 | CTATGTCCGATANNN | 1137 | CTATGTCCGATTTCT | chr6:30376959- | 5 |
| | | NNNNNNTATCGGAC | | TCTCATTATTGGACT | 30376991 | |
| | | ATAA | | TAA | | |
| WP_0154946 | 531 | TTATGTCCGATANNN | 1138 | CTATGTCCGATTTCT | chr6:30376959- | 5 |
| 05.1 | | NNNNNNTATTGGAC | | TCTCATTATTGGACT | 30376991 | |
| | | GTAA | | TAA | | |
| WP_0056103 | 532 | TTATGTCCGATANNN | 1139 | CTATGTCCGATTTCT | chr6:30376959- | 5 |
| 02.1 | | NNNNNNTATTGGAC | | TCTCATTATTGGACT | 30376991 | |
| | | GTAA | | TAA | | |
| WP_0932201 | 533 | TCACACGGGATANNN | 1140 | TCTCACAGGATACT | chr6:44113713- | 7 |
| 83.1 | | NNNNNNTACCCCGTG | | ACACTGTTACCCAG | 44113745 | |
| | | TGA | | TGTGA | | |
| WP_0655408 | 534 | AAAAACCACAGANNN | 1141 | AAAAACAACAGAAC | chr6:45110522- | 5 |
| 14.1 | | NNNNNTCTGTGGTTT | | CCCTTTTCAGTGCTT | 45110553 | |
| | | CT | | TCT | | |
| WP_0445439 | 535 | TATTGATGGATANNN | 1142 | TATTGATGGAAATT | chr6:48808288- | 4 |
| 06.1 | | NNNNNTATCCATCAA | | CTGCAATATCCATCC | 48808319 | |
| | | CC | | AAC | | |
| WP_0343966 | 536 | AAAGCCCGCAGANN | 1143 | AAAAGCCGCAGAG | chr6:71263114- | 6 |
| 20.1 | | NNNNNNCCTGCGGG | | GGCTCAGCCTGCCG | 71263145 | |
| | | CTTT | | GCTTT | | |
| WP_0484445 | 537 | TTATGACCGATANNN | 1144 | TTATGACGGATAAC | chr6:78996573- | 7 |
| 47.1 | | NNNNNTATCGGTCAT | | TGGGCATATTTGTC | 78996604 | |
| | | AA | | ATAA | | |
| WP_0034997 | 538 | TGGTACAACATANNN | 1145 | AGGTACAATATAAG | chr6:82026247- | 6 |
| 34.1 | | NNNNNTATGTTGTAT | | CCAAGATATGTTTT | 82026278 | |
| | | AA | | ATAA | | |
| WP_0010669 | 539 | TAGCATGTTACANNN | 1146 | TAGCAAGTTAAAGT | chr6:85617220- | 7 |
| 53.1 | | NNNNAGTAACATGCC | | ACGAAAGTAACATG | 85617250 | |
| | | A | | CAA | | |
| WP_0010669 | 540 | TAGCATGTTACANNN | 1147 | TAGCAAGTTAAAGT | chr6:85617220- | 7 |
| 42.1 | | NNNNAGTAACATGCC | | ACGAAAGTAACATG | 85617250 | |
| | | A | | CAA | | |
| WP_0154697 | 541 | ACCCCAATAAGANNN | 1148 | ACCCCAATGAGAAA | chr6:87787506- | 6 |
| 49.1 | | NNNNNTCTTGTTGGG | | ATACTTTCTCGTTGG | 87787537 | |
| | | GT | | GGA | | |
| WP_0121873 | 542 | ATATGTCCGATANNN | 1149 | ATATGTCTGACATTC | chr6:95103635- | 7 |
| 69.1 | | NNNNNNTATTGGAC | | CTTAGGTATTGGAC | 95103667 | |
| | | ATAG | | ATAA | | |
| WP_0565151 | 543 | GCTATGTTTTACANN | 1150 | AATATGTTTTACATT | chr7:106052119- | 5 |
| 34.1 | | NNNNNNNAATAAAA | | ACAACACAATATAA | 106052153 | |
| | | CATAGC | | CATAGC | | |
| WP_0514720 | 544 | CAAGTAGCGATANNN | 1151 | GAAGTAGAAATAG | chr7:116214710- | 8 |
| 36.1 | | NNNNNTATTGCTACT | | GAATTTATATTGCTA | 116214741 | |
| | | GG | | CTGG | | |
| WP_0163917 | 545 | CACCACTCCAGANNN | 1152 | CACCACTGCAGACT | chr7:125316538- | 5 |
| 64.1 | | NNNNNNTCTGGAGT | | GAAGTGCTCTGGTG | 125316570 | |
| | | GGTC | | TGGTA | | |
| WP_0529591 | 546 | TGTGATTCCATANNN | 1153 | TGTGAGTTCATACA | chr7:152786802- | 5 |
| 63.1 | | NNNNNNTATGGAAT | | TTTCCAATATGGTAT | 152786834 | |
| | | CACA | | CACA | | |
| AGC72343.1 | 547 | TAGCTTATGATANNN | 1154 | TAGCTTAAAATAGA | chr7:80489324- | 6 |
| | | NNNNNTATCAAAAG | | TTTACCTATCAAAA | 80489355 | |
| | | GTA | | GCTA | | |
| WP_1173167 | 548 | TAACCAACGATANNN | 1155 | TAACTAACAATATTC | chr7:81194736- | 5 |
| 04.1 | | NNNNNTATCGAAGG | | TTATTTATCGAAGTT | 81194767 | |
| | | TTA | | TA | | |
| WP_0207447 | 549 | TAACCAACGATANNN | 1156 | TAACTAACAATATTC | chr7:81194736- | 5 |
| 56.1 | | NNNNNTATCGAAGG | | TTATTTATCGAAGTT | 81194767 | |
| | | TTA | | TA | | |
| WP_0174370 | 550 | GGGCTACTAATANNN | 1157 | GGGCTACTTATAGA | chr7:82506117- | 3 |
| 96.1 | | NNNNNNATTTAGTAG | | ATTCTATATTTACTA | 82506149 | |
| | | CCC | | GACC | | |
| WP_0542920 | 551 | GAATTCATGCATANN | 1158 | GAATTAATGCATAG | chr7:8610238- | 7 |
| 66.1 | | NNNNNNTATGCATG | | GTTGATATATGCAG | 8610271 | |
| | | AAACC | | AAAACC | | |
| WP_0128621 | 552 | CATCAAACAATANNN | 1159 | AATCATACAATATA | chr7:86573735- | 5 |
| 44.1 | | NNNNTATTGCTTAAT | | TGACATATTGCTTA | 86573765 | |
| | | G | | ATT | | |
| WP_0226843 | 553 | GGATATGTGATANNN | 1160 | GGATATGTGATTAC | chr7:86824639- | 7 |
| 52.1 | | NNNNNTATCACATGT | | CATAATTCTCACATG | 86824670 | |
| | | TC | | TAC | | |
| WP_0767979 | 554 | GGTGTGCACAGANN | 1161 | GATGTGCAAAAACT | chr7:91397008- | 5 |
| 08.1 | | NNNNNNNTTTGTGCA | | TTGGCATTTTGTGC | 91397040 | |
| | | CACC | | ACACC | | |
| WP_0974526 | 555 | CTAACTTTAAATANN | 1162 | CTAACTTAAATTTTA | chr8:112961297- | 7 |
| 09.1 | | NNNNNNTATTTAAAG | | CTTTTCTATTTAAAG | 112961330 | |
| | | TTAG | | TTAG | | |
| WP_0162624 | 556 | CTAACTTTAAATANN | 1163 | CTAACTTAAATTTTA | chr8:112961297- | 7 |
| 25.1 | | NNNNNNTATTTAAAG | | CTTTTCTATTTAAAG | 112961330 | |
| | | TTAG | | TTAG | | |
| WP_0775433 | 557 | CTAACTTTAAATANN | 1164 | CTAACTTAAATTTTA | chr8:112961297- | 7 |
| 56.1 | | NNNNNNTATTTAAAG | | CTTTTCTATTTAAAG | 112961330 | |
| | | TTAG | | TTAG | | |
| WP_0321528 | 558 | CTAACTTTAAATANN | 1165 | CTAACTTAAATTTTA | chr8:112961297- | 7 |
| 54.1 | | NNNNNNTATTTAAAG | | CTTTTCTATTTAAAG | 112961330 | |
| | | TTAG | | TTAG | | |
| WP_0131603 | 559 | GCCCTGGTGAGANNN | 1166 | GCCCTGGTGAGAGT | chr8:143044855- | 6 |
| 48.1 | | NNNNTCTCACCAGGG | | CCCATGCCCACAAG | 143044885 | |
| | | C | | GGC | | |
| EHJ58476.1 | 560 | AGGGTGTTGATANNN | 1167 | ATGGTGATGATAAT | chr8:24207531- | 5 |
| | | NNNNNNTATCAACAC | | AATTCCTAATCAAC | 24207563 | |
| | | TGT | | ACTGT | | |
| WP_0398585 | 561 | AGGGTGTTGATANNN | 1168 | ATGGTGATGATAAT | chr8:24207531- | 5 |
| 63.1 | | NNNNNNTATCAACAC | | AATTCCTAATCAAC | 24207563 | |
| | | TGT | | ACTGT | | |
| WP_0535590 | 562 | ATCCCCCAGATANNN | 1169 | ATCTCCCAGATGAT | chr8:24330870- | 6 |
| 35.1 | | NNNNNNTATCTGGG | | CTAAGATTATCTGG | 24330902 | |
| | | GAAG | | AGAAG | | |
| SEC15746.1 | 563 | CAATGTCCGATANNN | 1170 | CAATCTCCTATACTT | chr8:32597229- | 8 |
| | | NNNNNNTATCGGAC | | TGATTTTATAGGAC | 32597261 | |
| | | ATTA | | ATTA | | |
| WP_0903301 | 564 | CAATGTCCGATANNN | 1171 | CAATCTCCTATACTT | chr8:32597229- | 8 |
| 26.1 | | NNNNNNTATCGGAC | | TGATTTTATAGGAC | 32597261 | |
| | | ATTA | | ATTA | | |
| WP_0250314 | 565 | CAATGTCCGATANNN | 1172 | CAATCTCCTATACTT | chr8:32597229- | 8 |
| 21.1 | | NNNNNNTATCGGAC | | TGATTTTATAGGAC | 32597261 | |
| | | ATTA | | ATTA | | |
| WP_0701745 | 566 | GCCCGCCTGAGANNN | 1173 | GTCTGCCTGAGAGG | chr8:40628155- | 6 |
| 36.1 | | NNNNNACTCAAGCG | | GTATAAACTCAAGA | 40628186 | |
| | | GGC | | GGGC | | |
| WP_0393287 | 567 | TAACTTCATATANNN | 1174 | TACATTTATATATAA | chr8:62042573- | 7 |
| 73.1 | | NNNNNTATATGAAGT | | ATGTATATATGAAG | 62042604 | |
| | | TG | | TTG | | |
| WP_1050800 | 568 | TAACTTCATATANNN | 1175 | TACATTTATATATAA | chr8:62042573- | 7 |
| 92.1 | | NNNNNTATATGAAGT | | ATGTATATATGAAG | 62042604 | |
| | | TG | | TTG | | |
| WP_0425961 | 569 | TTTGTATGTCTATANN | 1176 | TTTGTATGTATATAC | chr8:62870333- | 6 |
| 86.1 | | NNNNNNNTATAGAT | | ACAAAATATATGCA | 62870369 | |
| | | ATACTAA | | TATACTAA | | |
| WP_1132334 | 570 | TCACTATCGATANNN | 1177 | TCAATATCTATATAT | chr8:68696565- | 5 |
| 96.1 | | NNNNNNTATCGATA | | AGTTTATATCTATAG | 68696597 | |
| | | GTGA | | TGA | | |
| WP_1108804 | 571 | TCACTATCGATANNN | 1178 | TCAATATCTATATAT | chr8:68696565- | 5 |
| 04.1 | | NNNNNNTATCGATA | | AGTTTATATCTATAG | 68696597 | |
| | | GTGA | | TGA | | |
| WP_1200192 | 572 | TCACTATCGATANNN | 1179 | TCAATATCTATATAT | chr8:68696565- | 5 |
| 18.1 | | NNNNNNTATCGATA | | AGTTTATATCTATAG | 68696597 | |
| | | GTGA | | TGA | | |
| WP_0696942 | 573 | TCACTATCGATANNN | 1180 | TCAATATCTATATAT | chr8:68696565- | 5 |
| 92.1 | | NNNNNNTATCGATA | | AGTTTATATCTATAG | 68696597 | |
| | | GTGA | | TGA | | |
| WP_0921773 | 574 | TCACTATCGATANNN | 1181 | TCAATATCTATATAT | chr8:68696565- | 5 |
| 45.1 | | NNNNNNTATCGATA | | AGTTTATATCTATAG | 68696597 | |
| | | GTGA | | TGA | | |
| WP_0571937 | 575 | TCACTATCGATANNN | 1182 | TCAATATCTATATAT | chr8:68696565- | 5 |
| 06.1 | | NNNNNNTATCGATA | | AGTTTATATCTATAG | 68696597 | |
| | | GTGA | | TGA | | |
| WP_1335653 | 576 | TCACTATCGATANNN | 1183 | TCAATATCTATATAT | chr8:68696565- | 5 |
| 15.1 | | NNNNNNTATCGATA | | AGTTTATATCTATAG | 68696597 | |
| | | GTGA | | TGA | | |
| KSV89580.1 | 577 | TCACTATCGATANNN | 1184 | TCAATATCTATATAT | chr8:68696565- | 5 |
| | | NNNNNNTATCGATA | | AGTTTATATCTATAG | 68696597 | |
| | | GTGA | | TGA | | |
| WP_0583233 | 578 | TCACTATCGATANNN | 1185 | TCAATATCTATATAT | chr8:68696565- | 5 |
| 47.1 | | NNNNNNTATCGATA | | AGTTTATATCTATAG | 68696597 | |
| | | GTGA | | TGA | | |
| WP_1326658 | 579 | TCACTATCGATANNN | 1186 | TCAATATCTATATAT | chr8:68696565- | 5 |
| 65.1 | | NNNNNNTATCGATA | | AGTTTATATCTATAG | 68696597 | |
| | | GTGA | | TGA | | |
| WP_0696942 | 580 | TCACTATCGATANNN | 1187 | TCAATATCTATATAT | chr8:68696565- | 5 |
| 93.1 | | NNNNNNTATCGATA | | AGTTTATATCTATAG | 68696597 | |
| | | GTGA | | TGA | | |
| RWE07715.1 | 581 | TCACTATCGATANNN | 1188 | TCAATATCTATATAT | chr8:68696565- | 5 |
| | | NNNNNNTATCGATA | | AGTTTATATCTATAG | 68696597 | |
| | | GTGA | | TGA | | |
| WP_0115788 | 582 | TCACTATCGATANNN | 1189 | TCAATATCTATATAT | chr8:68696565- | 5 |
| 06.1 | | NNNNNNTATCGATA | | AGTTTATATCTATAG | 68696597 | |
| | | GTGA | | TGA | | |
| RWD51833.1 | 583 | TCACTATCGATANNN | 1190 | TCAATATCTATATAT | chr8:68696565- | 5 |
| | | NNNNNNTATCGATA | | AGTTTATATCTATAG | 68696597 | |
| | | GTGA | | TGA | | |
| WP_0964596 | 584 | TCACTATCGATANNN | 1191 | TCAATATCTATATAT | chr8:68696565- | 5 |
| 80.1 | | NNNNNNTATCGATA | | AGTTTATATCTATAG | 68696597 | |
| | | GTGA | | TGA | | |
| RWD87033.1 | 585 | TCACTATCGATANNN | 1192 | TCAATATCTATATAT | chr8:68696565- | 5 |
| | | NNNNNNTATCGATA | | AGTTTATATCTATAG | 68696597 | |
| | | GTGA | | TGA | | |
| WP_0162108 | 586 | CTACTTCCGATANNN | 1193 | CTACTTCAGATATA | chr8:92445006- | 7 |
| 37.1 | | NNNNNTATCGGAAG | | ACAAAATATCCGAA | 92445037 | |
| | | TAA | | GAAA | | |
| WP_0732881 | 587 | TAAGTTATGATANNN | 1194 | TAAGTTATGATAAT | chr9:102580364- | 5 |
| 06.1 | | NNNNNNTATCATAAC | | AGAAGTTTATAATT | 102580396 | |
| | | TTA | | ACTTG | | |
| WP_0927431 | 588 | TAAGTTATGATANNN | 1195 | TAAGTTATGATAAT | chr9:102580364- | 5 |
| 58.1 | | NNNNNNTATCATAAC | | AGAAGTTTATAATT | 102580396 | |
| | | TTA | | ACTTG | | |
| WP_0263515 | 589 | TAAGTTATGATANNN | 1196 | TAAGTTATGATAAT | chr9:102580364- | 5 |
| 76.1 | | NNNNNNTATCATAAC | | AGAAGTTTATAATT | 102580396 | |
| | | TTA | | ACTTG | | |
| WP_0893342 | 590 | TAAGTTATGATANNN | 1197 | TAAGTTATGATAAT | chr9:102580364- | 5 |
| 12.1 | | NNNNNNTATCATAAC | | AGAAGTTTATAATT | 102580396 | |
| | | TTA | | ACTTG | | |
| WP_0865970 | 591 | TAAGTTATGATANNN | 1198 | TAAGTTATGATAAT | chr9:102580364- | 5 |
| 10.1 | | NNNNNNTATCATAAC | | AGAAGTTTATAATT | 102580396 | |
| | | TTA | | ACTTG | | |
| WP_0925112 | 592 | TAACATAGGATANNN | 1199 | TAACATGAGATAAG | chr9:124694620- | 6 |
| 77.1 | | NNNNNTATCCCATGT | | CCACTAAATCCCAT | 124694651 | |
| | | TA | | GTTA | | |
| WP_0557393 | 593 | GGCTTAGGGATANNN | 1200 | GGTTTAGGGATACA | chr9:1707914- | 6 |
| 75.1 | | NNNNTATCTCTAAGC | | TGGGCAGTCTCTAA | 1707944 | |
| | | C | | GCC | | |
| WP_0580665 | 594 | TTTGTGGGGTAGANN | 1201 | TTTGTGGGGCAGG | chr9:1996891- | 4 |
| 17.1 | | NNNNNNTCTGCCCCA | | GAGATTTTCCTGCC | 1996924 | |
| | | CAAA | | CCACAAA | | |
| WP_0021875 | 595 | AATTACCGAATANNN | 1202 | AATTACAGAAGAGG | chr9:20409384- | 3 |
| 15.1 | | NNNNNNTATTTGGTT | | TGAAAGATATTTGG | 20409416 | |
| | | ATT | | TTTTT | | |
| WP_1276221 | 596 | TGACTATCGATANNN | 1203 | TGACTATCCATAAA | chr9:30689863- | 5 |
| 66.1 | | NNNNNNTATCGATA | | GAGGCTATAGCGAT | 30689895 | |
| | | GTGA | | AGAGA | | |
| WP_1012009 | 597 | ATTATTCTAGATANN | 1204 | ATTATTATAGTTACA | chr9:42127049- | 3 |
| 24.1 | | NNNNNNTATCTGGA | | TAGTTTTATCTGGA | 42127082 | |
| | | ATAAT | | AGAAT | | |
| WP_0683316 | 598 | TAGGTAGCGATANNN | 1205 | TATGTGGCTATATTT | chr9:7299781- | 6 |
| 37.1 | | NNNNNNTATCACTAC | | GTTTTCTATCACTAC | 7299813 | |
| | | CTA | | CTA | | |
| WP_0232747 | 599 | GCTTGTAAAATANNN | 1206 | CCTTGTAAAATATG | chr9:83685793- | 6 |
| 85.1 | | NNNNNNTATCTTACA | | AAATGGTTATCTGA | 83685825 | |
| | | AGC | | CAATC | | |
| WP_0184094 | 600 | CCATGTCCGATANNN | 1207 | CCATTTCAGATAGA | chrX:109132372- | 6 |
| 63.1 | | NNNNNNTATCGGAC | | GAACATGTATTGGA | 109132404 | |
| | | ATGA | | CATGA | | |
| WP_0103052 | 601 | GACTTATCTAATANN | 1208 | GACTTATTTAATAA | chrX:123330942- | 6 |
| 36.1 | | NNNNNNTATTAAATA | | ATAGACTTATTTAAT | 123330975 | |
| | | AATC | | AAATA | | |
| WP_0087370 | 602 | GTGGTGGGCAGANN | 1209 | ATGGTGGGCATAG | chrX:123955891- | 6 |
| 17.1 | | NNNNNNNTTTGCCCA | | GACTATTGTATGCC | 123955923 | |
| | | CCAT | | CACCAT | | |
| WP_0065260 | 603 | TTGAGTGTTACANNN | 1210 | TTAAGTGTTACACA | chrX:140388413- | 7 |
| 94.1 | | NNNNNNTGTTACACT | | TATTTTATTTTACCC | 140388445 | |
| | | CAC | | TCAC | | |
| WP_1276571 | 604 | TAAGATACGATANNN | 1211 | TAACATGCGATATA | chrX:15022673- | 5 |
| 23.1 | | NNNNNNTATCGTATC | | TACTATATATCGTAT | 15022705 | |
| | | TAA | | ATAA | | |
| WP_0718572 | 605 | AGCTCCTTTATANNN | 1212 | AGCTCCTCTATGATT | chrX:16696196- | 6 |
| 25.1 | | NNNNNTATAAATCAG | | AAAACTAAAAATCA | 16696227 | |
| | | CT | | GCT | | |
| WP_1076761 | 606 | TCACTAGCGATANNN | 1213 | TCACTAGAGATAGA | chrX:21966067- | 5 |
| 28.1 | | NNNNTATCGATAGTG | | CTCTTTATGCATAGT | 21966097 | |
| | | A | | GA | | |
| WP_0031322 | 607 | AAGTTACTGACANNN | 1214 | AAGTTACTGAGATG | chrX:41824012- | 6 |
| 98.1 | | NNNNTGTCAGTAACT | | CAAGATGTCAAAAA | 41824042 | |
| | | C | | CTC | | |

Non-limiting examples of amino acid sequences of tyrosine recombinases are provided in Table 1, column 1 by accession number. Table 1 further provides, in column 2, exemplary native non-human (e.g., bacterial, viral, or archaeal) recognition sequence(s) to which a given exemplary tyrosine recombinase binds. Each of the native recognition sequences listed in Table 1 typically comprises three segments: (i) a first parapalindromic sequence, (ii) a spacer (e.g., a core sequence) that generally does not include a defined nucleic acid sequence, and (iii) a second parapalindromic sequence, wherein the first and second paralindromic sequences are parapalindromic relative to each other. Table 1 further provides, in column 3, exemplary recognition sequence(s) for each exemplary tyrosine recombinase in the human genome. Generally, the human recognition sequences listed in column 3 of Table 1 each comprises three segments: (i) a first parapalindromic sequence, (ii) a spacer (e.g., a core sequence) that generally includes a defined nucleic acid sequence, and (iii) a second parapalindromic sequence, wherein the first and second paralindromic sequences are parapalindromic relative to each other. Table 1 includes, in column 4, genomic locations of the exemplary human recognition sequences in the human genome.

TABLE 2
Amino acid sequences of the tyrosine recombinases of Table 1.

| SEQ | |
|---|---|
| ID | |
| NO: | Bidirectional Tyrosine Recombinase |
| 1215 | WP_006717173.1 |
| | MAKKVKPLVDTEIKKAKASDKPYTLTDGYGLFLIISPTGSKSWRFNYYRPLTKKRAKIALGVYPAITLSK |
| | ARELREQYRQLLALKIDPQEHIKQNELLQLQRQQNTFFAIATQWKQKKVSEIKEATLKSRWRTIEKYVFP |
| | YLGDNPIADITPQQLHDIAMPLFERGVSHTGKLVIALVNEIMGFAVNKGVIEFNKCVNVSKAFNVNRTTH |
| | HPTIRPEQLPEFMSALRNSHIDLMVKYLIEFSLLTMTRPSEAANALWDEIDFEKSLWNIPAERMKMKKAF |
| | TVPLSPQVLKILNKLKNISGRSRFIFOSQRYPERSLHSSSANAAIKRVGYKDQLTSHGLRSIASTYLSET |
| | FTEMNLEILEACLSHQSKNQVRNAYNRSTYLEQRKLLMNAWGNFVEECMKKSI |
| 1216 | WP_006718580.1 |
| | MLTDTKIKSLKPKDKVYKVADRDGLYVSVSTAGTITFRYDYRINGRRETLTIGKYGADGINLAEARERLM |
| | IARKQVSEGISPATEKRAERNKIRNADRFCVFAEKYLADVQLADSTKALRVATYERDIKDTFGNRLMTEI |
| | TADEIRSHCEKIKERGAPSTAIFVRDLIANVYRYAIQRGHKFANPADEIANSSIATFKKRERVLTPREIK |
| | LFFNTLEETQSDFALKKAVKFILLTMVRKGELVNATWNEVDFKNKVWTIPAERMKAKRAHNVYLSEQALD |
| | LIIAFQIYSEGSPYLLPGRINRRQPIANSSLNRVIANCIKFINKDEQRIDEFTVHDLRRTGSTLLHEMGE |
| | NSDWIEKSLAHEQQGVRAVYNKAEYAEQRKEMMQRWADQVDEWINDNSL |
| 1217 | WP_006719234.1 |
| | MPKITKPLTNTEVERSKPKAKEYTLTDGYGLFLLVLPTGVKSWRFNYIRPLTKKRTKVSLGTYPALSLAQ |
| | ARSIREEYRSLLAQGIDPQEHKEQEQKAAIEHIENSLLSVANRWKAKKVQKVEAETLKKDWRRMEIYLFP |
| | FIGDMPINEILPKVVIEALESLYNQGKGDTLKRTIRLLNEVLNFAVNYGLIAFNPCLRINEVFNFGKSTN |
| | NPAITPKELPELIKAVMYSSAAIQTKLLFKFQLLTMVRPAEASNATWSEIDFKKSLWTIPANRMKKRHPF |
| | VIPLSSQAMAILNKMKSISVKSEYVFQSWIKSNQPMSSQTINKMLVDLGYKNKQTAHGLRTIGHTYLADL |
| | RIDYEVAEMCISHKTGTQTGKIYDRADFLEQRKPVMQLWGDYVEQCER |
| 1218 | WP_109859198.1 |
| | MNDLTLLDLFLNELWIGKGLSPNTVQSYRLDLTALCDWLGERKLSLLDLDSVDLQTFLGERVEQGYKATS |
| | TARLLSAIRKLFQYLYQEKYRTDDPSAVLSSPKLPSRLPKYLTEQQVTDLLNVQSLEQPIELRDKAMLEL |
| | LYATGLRVTELVSLHTDSISLNQGVVRVIGKGNKERIVPMGEEATHWVKQFMLFARPILLDGQSSDVLFP |
| | SRRGTOMTRQTFWHRIKHYAVLAEIDSNMLSPHVLRHAFATHLVNHGADLRVVOMLLGHSDLSTTQIYTH |
| | VAKERLKRLHERYHPRG |
| 1219 | WP_006717195.1 |
| | MNDLTLLDLFLNELWIGKGLSPNTVQSYRLDLTALCDWLSERKLSLLDLDSVDLQTFLGERVEQGYKATS |
| | TARLLSAIRKLFQYLYQEKYRTDDPSAVLSSPKLPSRLPKYLTEQQVTDLLNVQSLEQPIELRDKAMLEL |
| | LYATGLRVTELVSLHTDSISLNQGVVRVIGKGNKERIVPMGEEATHWVKQFMLFARPILLNGQSSDVLFP |
| | SRRGTQMTRQTFWHRIKHYAVLAEIDSNMLSPHVLRHAFATHLVNHGADLRVVQMLLGHSDLSTTQIYTH |
| | VAKERLKRLHERYHPRG |
| 1220 | WP_005715799.1 |
| | MQNELQKYLTYLRIERQVSPHTLTNYQHQLVRVIAILQDAGIQQWQQVTLSVVRYVLAQSSKQDGLKEKS |
| | LALRLSALRRFLSYLVYQGQLKVNPAVGVSAPKQPKHLPKNIDRDQIQLLLANDSKEPIDIRDRAMIELF |
| | YSSGLRLSELQGLNLNSINLRVREVRVIGKGNKERIVPLGRYASHAIQQWLKVRLLFNPKDDALFVSQLG |
| | NRMSTRTIQMRLERWGIRQGLNSHLNPHKLRHSFATHMLEASSDLRAVQELLGHSHLSTTQIYTHLNFQH |
| | LADVYDAAHPRAKRKK |
| 1221 | WP_120166565.1 |
| | MESIVLKFIEYLKNEKELSKNTIESYNRDLRQFKEYISDNKINDITGVNKTAIIKYLMHLQKIGKSTSTV |
| | SRNLASLRSFYQYLLNKGIINQDPTLNLQSPKPEKKLPDILTPKEVDILLRQPDITTSKGIRDKAMLELL |
| | YASGIRVSELIDLNLEDINLDLGYLVCSKNNSNERIIPIGKIALNILKTYIKDYRKKFIKDKNVKSLFVN |
| | YHGNKMTRQGFWKIVKSYAKKANINKKITPHTLRHSFATHLLQNGADLKSVQEMLGHSDISTTQVYAQIT |
| | KNNIKEVYKKAHPRA |
| 1222 | WP_061329756.1 |
| | MRVQEVKLENNQRRYLLVDDIGLPVIPVAKYLKYIDNSGKSFNTQKTYCYSLKLYFEYLQEIAVDYRSVN |
| | INILSDFVGWLRNPYANNKVVNLKPTIAKRTEKTVNLTVTVVTNFYDYLYRTEELNNDMIDKLMKQVFTG |
| | GNKHYKDFLYHINKDKPTNKNILKIKEPRRKIKVLTKEEIQSVYNATTNIRDEFLIKLLFEAGLRMGEAL |
| | SLFIEDIIFDHNNGHRIRLVNRGELPNGARLKTGEREIHISQELIDLFDDYAYDILDELEIDTNFVFVKL |
| | RGKNKGTPLEYQDVSDLFKRLKKKTGIDVHAHLLRHTHATIYYQTTKDIKQVQERLGHSQIQTTMNMYLH |
| | PSDEDMRANWEIAQPSFKITKRGTNDN |
| 1223 | WP_010497271.1 |
| | MSVIKNFPAHAKPYQATYTNGSGRGRIRKIKSFVSSKDAQLWLKQMETNFINGETYAKSQMLFVDYFQEW |
| | YRLYKAPVVSPPTLDSYYNSWRHFKEHGLGHVKMENLTRDKIQTYLNDLAYAKETTRKDLNHLRACLRDA |
| | YDDGVISRNPAAGTLHVIADPAKSKSKDRKFMAETDFRKVQDFLLNYNYRLSDVNRAVLLVISQTALRVG |
| | EALALRYDDLNQLNCTIRVDESWDAKHLMFGKPKTESGYRTIPVSRQAMKKIITWQNFHRRELFRRGIPN |
| | PGNLLFLNRQKNLPRASAINSCYHQLQLRLGIEAKFSTHTMRHTLASILLGSGEVSIQYISYFLGHANVA |
| | ITQKYYIGLLPEQVEKEDQEVVKIVGAL |
| 1224 | WP_038150996.1 |
| | MASYSISTRQKDKNWQVIVSYKDRYGRWRQKSKQGFLTKRTAKDYGDIIVKEIKENLLLTNNEELANITF |
| | LEFSKIYFNDVKDTLRANSLITYQNLIKYVSPLYNLQLHEITPLIINTTLKNITSSTTSKKFIVSILKRI |
| | FSHAIKEYNLLSKNPVTATVPSEKINKPIRVITNEELDLYYNTISTSNQIYVAIKILQYTGIRIGELFAL |
| | TQDDIDYKEMTISINKQFVTVGKNKNGIGPLKTKNSYRTIPIPKSLAVILSEYTSTCTTDRIITYKSTNA |
| | LRKHIKKHINNHAPHDFRHTYATKLLANGMDVKTVAALLGDTVTTVINTYIHYSDEMRQSAKKDIQRIFD |
| 1225 | WP_038150898.1 |
| | MKLIEKMKGATKRPYVAYKIVGYYRTYDEAVDALQNASKKYTLYQLYTSWLSTHRNSVTSTTISNYHSAI |
| | AHATSIHNTYIDEITYIQLQSIIDTMLRNHLSYSSCKKVRSLLSQLFDYAIINNLISTNYAHYVKIGTNT |
| | PVRPHVTFTTRQINKLWRLSSPLRDIPLILLYTGMRATELINLTSKNVNRKQRTIRITSAKTKAGIRTIP |
| | IHDRIYDIIINRLDSQYVIEECRTYQSLAHQFNQAMKAINAKHTTHDCRHTFATRLDDVGANYNAKRLLL |
| | GHASSNVTDGVYTHKSLVQLRKAIRMLK |
| 1226 | WP_017740000.1 |
| | MRSKKGEVSISLRNGNYQLYWRYKGEKFYLSPGLSESKVNAIAVEKLANQIKLDIIFENFDETLKKYKPE |
| | KTVEKVNKAKKELDIDSRLENYFTVRGIKSKGTKDVYLAVVKRYKSFFYGKKEPNLTDLQKFLEHLKNEG |
| | LSLVTIKSYLIKLAAVFDNTEPWKIIKKQIKPNPVQPKPFTKEEVFSIIENCPEHYRNFVKFLFYSGCRI |
| | GEAINLKWENVTEDFSSVWILADKTKKARKLILTEELKAVIRDSKDKAKSNIYVFTAKTRKSEQVSRKYF |
| | CDYIWKPLLIKLNISYRKPYYTRATMISHSLEAGLSPLKLAKITGHSQSTMWNHYYADLGIENKIPDIFN |
| | QE |
| 1227 | WP_017744257.1 |
| | MHIVTFKGRIRFNLPRQWFGGKQQQWNLKLEATEVNMALASRVARRLEMDFQDGKLTVALPDGSTAFNKE |
| | HYNKVLAEYNIEGNLRTDLKLITGGLPSDEIPPKPQMSLLDVWDMYCEHKFKNGKLAKTTYGQYKSQYRN |
| | YLISAMEANGGEDAIKIKNWLLENRNREIVCKILSGLEQAYKVALRQKLVSFNPYEGIMEDVSRIKRETE |
| | IDVTKESDEDLLNKSKAYTWDEAQVIMEYLKDSPSYGHWYHFVAFKFLTGCRTGEAIGLCWMDVKWDGQC |
| | VVISKTWTRLKFYKPTKTEKEKRVFPMPVDGELWNLLKSLPQGNPSEPVFKSKNEKMIHIDIFGTAWRGR |
| | ESKRNKGIIPTLIEQGKLSKYLPPYNTRHTFVTHQIFDLGRDEKIVSAWCGHSEAVSSKHYQDIADRASQ |
| | INPELPVNNQQVQQVSNEMDEMRNIIKSLQEQLKTQSEVIASLQEQLKNK |
| 1228 | WP_O17746151.1 |
| | MYETGKPSSRVPKITPRNNNGGIIIRFQYQGKQYSISPGGKYSDKLAIANANKIASQIKTDILAGYFDPT |
| | LEKYQPKVKQPDNVVSINKDVALSLKELWEQYKLAKRASVAETTQKEKWSQIDRCLTKVSPEILNPENAR |
| | LLIPELLKAYSSTTLERIINDIHACSHWAFETGLISINPWRRLKQQLPDKPOSSRTKKAYSRDEVNAIIQ |
| | AFRGDWYCNSKSAFKDSWYADFIEFLFLTGCRPEDAIALTWEQVKERVIVFDKAYSCGVLKSTKNNKARM |
| | FPITPQIRELLDRRLTSVSTIPTKLVFPAQNGNYINLRNFTQRYTKRIIENLVSEGKVKQYLPTYNLRNT |
| | SITHYLRQGVDIATVAALMETSEEMINQHYWSPDDDIINNNVQLPEI |
| 1229 | WP_126045042.1 |
| | MNNFININNDKNSIIVANLQEKVKDYARHAFAKNTIKNYQSDWKIFCTWCESLNINPLNITHNTLIAYIT |
| | FLAEENYKASTIQRKISAIYKYCETKNIHINLQDKEFKIVWQGIRRKIGIVKQGKDPILLKDLEDILQHI |
| | SKNTHMGIRDRALLTFGWFSAMRRSELVKLNWQDISFIKEGIIINIRQSKTDKFGEGQKIAILKRKIFCP |
| | IKHLKAWQKINNNEAVFCSVNKADKVTGIRLSCIDVARITKKHSAKIDFDTSKIAGHSLRRGFVTTAVSS |
| | GIRNHIIMKTTRHKSSKMIDDYTHDNSLLENNATNMIITSNSSSKKFNSILKNYLQFKAAYKLYNKVKTN |
| | IKKLYFFCIPPTL |
| 1230 | XP_012333305.1 |
| | MHHIGKALCFFFILNCMDKTTSFFINKVHIFHLTTFRNGGNLTHIRKKCPNSMGVTVKRKGACLNSHDEE |
| | EAEDIDEAETEDEEEMQEEDELEETSDVDETDASDGRLSPRSTKKVTTGGKRVKAGKIKKRKRKKKTTNA |
| | AKCRTCNKILKPRVKFCVHCGTNVSVEKIKLKKYIEDIYLPLRKEEVSYNTYRVEKGFWNDILPKLGKYE |
| | LHELGPNNWESFLKYLKWKNCSPRTMALYQSTYQQSLKYALYRDYLKSVHNFRKIKNSTIPRRKITPLSP |
| | KEIELLLINSGDMHRAIFALSIGIGLRPSEVLRILWEDVNFEKKEIFIKGQKTKYSNTAIPMTNFAYNEL |
| | VKWWEIEKKPLKGLCFYSETIKNKFNYTNTKTPLKTFKTALKGAAKRAGLEISEDGKKRRIFPYLLRHSF |
| | ATIAATSNPPVPLPVAQAIMRHSSSKMLLDTYTKAGNNIIRDGLDNFKI |
| 1231 | WP_073025039.1 |
| | MAIHKPVALYPTFKELKEIPLDEFPELSSFLSSGPGWRKQSWLWGQEFLSYIGRNKSQHTYTRFRSEIEK |
| | FLLWSFVIKESPCDEFRKTDILDYADFCWKPPQTWISLTNHDKFQPKGDGTYIQNKAWSPYRLVVSKGDN |
| | STPDKKKYRPSQQTLRATFTAIIAFYKYLMDEEYCVGNPAQLAKKDCRHFIKDAQVKDVKRLSEEQWLFL |
| | LETVTAMADDNSRFERNLFLIAALKTLFLRISEFSERPDWIPVMGHFWEDTDKNWWLKVYGKGRKLRDIT |
| | VPSSFMPYLKRYRLYRGMSSLPLDGEKHPIVEKLRGSGGMTARQLSRLVQEVFDHAYETMKKQQGEEIAR |
| | KFREVSTHWLRHTGASLEIERGRALKDLSEDLGHSSMATTDTVYVQSEDRKRAESGKNREV |
| 1232 | WP_007635552.1 |
| | MLLKKPVPLYPPYLDLCDFDFKDYPELKEIFSSNESWWLEQFNWGKVFLNYIGRNKSTHTYDRFRNDVER |
| | FLLWSFIEKKKPIDQLRKTDLLEFADFCWHPPVSWIGTSNQERFKIMNGYSCANEFWFPYKIQAPKSQKT |
| | QFIIDKKKYRPSQQTLSSMFTAIIVFYNYLMAEDFCIGNPAQIAKKDCRHFIIDSQVKEIKRLTGSQWQY |
| | VLDTAVEMADDNPVFERNLFVIVALKTLFLRISELSERTNWSPTMGHFWQDDDENWWLKIFGKSRKIRDI |
| | TVPIDFLPFLERYRISRGLIGLPSSNENLVLVEKIRGQGGMTSRHLRRLVQSVLDQAHENMRTSEGENKA |
| | LKLKEASAHWLRHTGASMEIERGRPLKDISEDLGHASMATTDTVYVQSENKKRAESGKQRKVD |
| 1233 | WP_058958135.1 |
| | MTELVPLTELQMNRSGDIAERLRQFVQDKEAFSPNTWRQLLSVMRICNQWSEENQRSFLPMSADDLRDYL |
| | TFLAESGRASSTVTSHAALISMLHRNAGLPVPNTSPQVFRAMKKINRVAVMSGERAGQAVPFRLSDLLAL |
| | DRQWSGADSLQARRDLAFLHVAYATLLRISELSRLRVRDVMRAGDGRIILDVAWTKTVVQTGGLIKALST |
| | RSTQRLEEWLDASGLSGQPDAYLFTAVHRSGRSLPAEKPMSTRALEQIFERAWRCAGKAGGVKANKNRYT |
| | GWSGHSARVGAAQDMADKGYPIARIMQEGTWKKPETLMRYIRHVEAHKGAMVEFMEQHADGTLPD |
| 1234 | WP_090967054.1 |
| | MSELVPITSLEASRNSDDITERLRQFVQDKEAFSPNTWRQLLSVMRICNRWSEDNQRSFLPMSAEDLRDY |
| | LSFLAESGRASSTITSHAALLSMLHRNAGLPVPNVSPLVFRTMKKINRVAVMNGERAGQAVPFRLTDLLA |
| | LDGEWSGSESLQALRDLAFLHVAYATLLRISELSRLRVRDVMRAGDGRIILDVAWTKTIVQTGGLIKALS |
| | ARSTQRLEKWIEASGLFSQPDAYLFSAVHRSGRALIAEKPISTRALEQIFSRAWLTAGKSGAVKANKNRY |
| | TGWSGHSARVGAAQDMADKGYPIARIMQEGTWKKPETLMRYIRHVDAHKGAMVEFMEQHADTDFPG |
| 1235 | WP_010365336.1 |
| | MLSPLVDTLKQLRYQIAHIEDGTLTNEYPELESFLSHVVRSVPNARDDIEFLYQFLYVYGRKSEATFNRF |
| | RNELERFYLWAWEWRALSVFELKREDIEAYVEFVVEPDNRWISDSVQWRFKDHEGLRVVNKLWRPFAFKE |
| | NGVSQQTFSAMFTALNVFYKFAILEEKTFTNFIPVVKKNSPYLIVQSQIKLPDTLSNLQWEYVFGVTRDK |
| | CEENPSLERNLFTLACLKGLYLRISELSERPQWSPVMSHFWQDPDGFWYLRIMGKGNKLRDVTLSEDFII |
| | YLRRYRQYRALPALPRVDEPHPIIHKLRGQGGMNVRQIRRIVQQSFDLAVDSLAADGFSDESEQLKAATA |
| | HWLRHTGATHDAQHRPLKHLSEDLGHAKIATTDQIYIQTNIKDRAKSGSKRKL |
| 1236 | WP_016392893.1 |
| | MARTVTPLSDSKCEAAKPRDKDYKLFDGQGLFLLIKPSGVKTWRFKFIRPDGREGLATFGNYPALGLKAA |
| | RDRRADFLELLAAGRDPIEAGKVAKMDAANARINTFEALARVWHSTCARKWKPHHAATVLRRMELHLFPS |
| | LGARPIADLKARDLLAPLKAAERRDTLETASRLRQYIAGILRMAVQHGIIDINPANDLQGATATRKTAHR |
| | PALPLERLPELLTRMDAYNGRQLTRMAVQLSLLVFTRSSELRFARWDEIDFERALWTIPAERQPIEGVKH |
| | STRGAKMATPHLVPLSRQALALLAEVHQLTGNYELVFAGDHHYWKPMSENTVNAALRRMGYDTKADVCGH |
| | GFRAMACSSLVESGLWSRDAVERQMSHQERNGVRAAYIHKAEHIEERRLMCQWWADYLDASRKKYATPYD |
| | FANCGRDAGNVVSIMRG |
| 1237 | WP_047824597.1 |
| | MAPETALDDDRPDRGEALSLSRDLALVAHGPGAGPSPELLAAYVRAAAPNTLRAFRSDVLAFDAWCRSRG |
| | EKSIPASPQIVADWLSTRASGGAAPASLSRYKASIARLHRLCGLADPTGDELVRLTLAAYRREKGVAQKQ |
| | ARALRFRGAVKDPLSDTPRGINVRAVLASLGDGLTDLRDKALLSLAYDTGLRASELVAVQVEDIGEAIDA |
| | DARLLAIPRSKGDQEGEGATAYLSPRTVRALEAWLKAAVIGEGPVFRRVVVRRYAARQARKARNGKERGW |
| | NARWVPERFAAKDAEPVRIESDVGEGALHPGSITPLIRSMLRRAFDVGAFGDLDAATFEKQVREISAHST |
| | RVGVNQDYFAAGEDLAGIMDALRWKSPRMPLQYNRNLAAEQGAAGRLLGKLR |
| 1238 | WP_046407494.1 |
| | MNALLPFADDVTGSGIVAIDADVIDAARRAMSPNSWRALRADIRVFAGWCAARGLMTLPALPATVATFLA |
| | DQADHGKKAATLARYTASIARLHALADQPDPTRTERVRLELKAQRRALGVRQRQARGLRFRGEVADPLAA |
| | AGPVGVCVEAMLAATGDDLPGQRNRALLSLAFDTGLRRSEIVAIRWPHVERGGAGGGRLFVPRSKADQEG |
| | AGAYAYLSARTMTALGEWRAACGGRSDGALFRRLHRTRDKSGADIWSVGAALSAQSVTLIYRAMLDAAHA |
| | AGLLGMIDSADFDIWRASLTAHSTRVGLTQDLFASGQDLAGIMQALRWKSPAQPARYAQALAVESNAAAK |
| | VVGKL |
| 1239 | WP_003712523.1 |
| | MKQLVLPIKDSNVLHEVQDTLLNNFRFGRRNYTIFQFGKATLLRVSDVLALRRNEIFTDDGLIKKNAYIR |
| | DKKTNKPNILYLKPIKQDLSQYYSWLDENSIHSEWLFPSLKHPERHISEKQFYKEKQFYKIMAKTGDLLN |
| | INYLGTHTMRKTGAYRVYTQTNFNIGLVMSLLNHSSEAMTLKYLGLDQVSREQMLDEVKFD |
| 1240 | WP_005027658.1 |
| | MPLTDTHIRSLKPDVKPRKYFDGGGLFLFVPANGSKLWRMAYRFDGKSKLLSFGEYPTISLKDARERREE |
| | AKRMLSKGIDPSDHKRQLRQARAIAERDSFQNIAREWHETRMAEFSEKHQGTVMYRLETYIFPAIGKTHI |
| | AKLETRDVMEVVKPLEQRGNYETSRRVLQIISQVFRYAVITGRAKHNVAADLRGALRPRKTVHRAAVLEP |
| | EKVGQLLRDIDAYEGYFPLVCALKLAPLVFTRPTELRAAQWKEFDLEAGEWRIPAERMKMRRQHLVPLSR |
| | QAMSILRELQKCSGEGKYLFPSIRTEARSISDATMLNALRRMGYQKHEMSVHGFRSIASTLLNELGYNRD |
| | WIERQLAHGEQDEVRAAYNYAEYLPERRKMMQAWADYLDGLRNTQQKRIREEA |
| 1241 | WP_021170377.1 |
| | MNSNDKDFVLRKNNFIQNNKKLSIKSKKRLQKSKSDNTLRAYEADWMDFYDWCTYHSLQALPAEPETIVN |
| | YINDLADHAKANTVSRRVSAISENHKAAGCVDNNPCRGGLVRNALDAIRREKGTLQRGKAPILMEDLRNI |
| | TAYFDTTDIAGIRDKALLLVGFMGAFRRSELVQIDIEDLTFTQEGVIILVAQSKGDQLGQGAQVAIPYSS |
| | NLDICAVTALKSWIHRANLASGPLFRPVNKYKQIRNRRLTNQSVAIIVKKYTKLSGLNPDNFAGHSLRRG |
| | FATSAAQHDVDERSIMQQTRHKSEKMVRRYIEQGNLFKNNPLNKMF |
| 1242 | WP_015169902.1 |
| | MAKNNRHGQAEILKDLELDRIYRQLQSDSHRLFFNIARYTGERFGAICQLQVCDVYVCYSGIKEPLNEIT |
| | FRAMTRKASPNGERKTRQAYVCDRLREYLSSYRGELGKVYLFPSSIKKDDPITFSAADKWLRTAVDRAGL |
| | EHRGISTHTFRRSFITKLYEEGALDIYAIQQLIGHASILTTQRYLGVSKQKIQSAMNRIYN |
| 1243 | WP_089415106.1 |
| | MSELVPLTPLTVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLSDLLAL |
| | DEEWSGSDNLQALRDLAFLHVAYATLLRISELSRLRVRDVMRAGDGRIILDVAWTKTIVQTGGLIKALSA |
| | RSTQRLEEWIEASGLSSQPDAWLFTAVHRSGRPLIAEKPMSTRALEQIFSRAWRTAGKEGAVKANKNRYT |
| | GWSGHSARVGAAQDMADKGYPIARIMQEGTWKKPETLMRYIRHVDAHKGAMVEFMEQYGDPDYPG |
| 1244 | WP_022624268.1 |
| | MSELVPLTPLTVDRNSDITERLRQFVQDKEAFSPNTWRQLLSVMRICNRWSEDNQRSFLPMSADDLRDYL |
| | SFLAESGRASSTVTSHAALISMLHRNAELPVPNVSPLVFRTMKKINRVAVINGERAGQAVPFRLSDLLAL |
| | DKEWSGSDNLQALRDLAFLHVAYATLLRISELSRLRVRDVMRAGDGRIILDVAWTKTIVQTGGLIKALSA |
| | RSTQRLEEWIEASGLSSQPDAWLFTAVHRSGRPLIAEKPMSTRALEQIFSRAWRTAGKEGAVKANKNRYT |
| | GWSGHSARVGAAQDMADKGYPIARIMQEGTWKKPETLMRYIRHVDAHKGAMVEFMEQYGDPDYPG |
| 1245 | WP_046103089.1 |
| | MTELVPLTELQMNRSGDIAERLRQFVQDKEAFSPNTWRQLLSVMRICNQWSEENQRSFLPMSADDLRDYL |
| | TFLAESGRASSTVTSHAALISMLHRNAGLPVPNTSPQVFRAMKKINRVAVMSGERAGQAVPFRLTDLLAL |
| | DRQWSGADSLQARRDLAFLHVAYATLLRISELSRLRVRDVMRAGDGRIILDVAWTKTVVQTGGLIKALST |
| | RSTQRLEEWLDASGLSGQPDAYLFTAVHRSGRSLPAEKPMSTRALEQIFERAWRCAGKAGGVKANKNRYT |
| | GWSGHSARVGAAQDMADKGYPIARIMQEGTWKKPETLMRYIRHVEAHKGAMVEFMEQHADDALPD |
| 1246 | WP_069027120.1 |
| | MSELVPLTPLTVDRNSDITERLRQFVQDKEAFSPNTWRQLLSVMRICNCWSEDNQRSFLPMSADDLRDYL |
| | SFLAQSGRASSTVTSHAALISMLHRNAGLPVPNVSPLVFRTMKKINRVAVINGERAGQAVPFRLTDLLAL |
| | DKEWAGSDNLQALRDLAFLHVAYATLLRISELSRLRVRDVMRAGDGRIILDVAWTKTIVQTGGLIKALST |
| | RSTQRLEEWIEASGISSQPDAWLFTAVHRSGRPQIAEKPMSTRSLEQIFSRAWRTAGKEGAVKANKNRYT |
| | GWSGHSARVGAAQDMADKGYPIARIMQEGTWKKPETLMRYIRHVDAHKGAMVEFMEQYSDPDYPG |
| 1247 | WP_010671927.1 |
| | MSELVPLTPLTVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLSDLLAL |
| | DKEWSGSDNLQALRDLAFLHVAYATLLRISELSRLRVRDVMRAGDGRIILDVAWTKTIVQTGGLIKALSA |
| | RSTQRLEEWIEASGLSSQPDAWLFTAVHRSGRPLIAEKPMSTRALEQIFSRAWRTAGKEGAVKANKNRYT |
| | GWSGHSARVGAAQDMADKGYPIARIMQEGTWKKPETLMRYIRHVDAHKGAMVEFMEQYGDPDYPG |
| 1248 | WP_109653747.1 |
| | MSELVPLTPLTVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLTDLLAL |
| | DKEWAGSDNLQALRDLAFLHVAYATLLRISELSRLRVRDVMRAGDGRIILDVAWTKTIVQTGGLIKALST |
| | RSTQRLEEWIEASGISSQPDAWLFTAVHRSGRPLIAEKPMSTRSLEQIFSRAWRTAGKEGAVKANKNRYT |
| | GWSGHSARVGAAQDMADKGYPIARIMQEGTWKKPETLMRYIRHVDAHKGAMVEFMEQYSDPDYPG |
| 1249 | WP_134161939.1 |
| | MSELVPLTPQTVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLTDLLAL |
| | DKEWAGSDNLQALRDLAFLHVAYATLLRISELSRLRVRDVMRAGDGRIILDVAWTKTIVQTGGLIKALST |
| | RSTQRLEEWIEASGISSQPDVWLFTAVHRSGRPLIAEKPMSTRSLEQIFSRAWRTAGKEGAVKANKNRYT |
| | GWSGHSARVGAAQDMADKGYPIARIMQEGTWKKPETLMRYIRHVDAHKGAMVEFMEQYSDPDYPG |
| 1250 | WP_111534863.1 |
| | MSELVPLTPLTVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLSDLLAL |
| | DEEWSGSDNLQALRDLAFLHVAYATLLRISELSRLRVRDVMRAGDGRIILDVAWTKTIVQTGGLIKALSA |
| | RSTQRLEEWIEASGLSSQPDAWLFTAVHRSGRPLIAEKPMSTRALEQIFSRAWRTAGKEGAVKANKNRYT |
| | GWSGHSARVGAAQDMADKGYPIARIMQEGTWKKPETLMRYIRHVDAHKGAMVEFMEQYGDPDYPD |
| 1251 | WP_128085508.1 |
| | MRESASLINLTVNRSDDIAERLRQFVQDKEAFSPNTWRQLISVMRICHQWSEVNQRTFLPMRAEDLRDYL |
| | AFLAESGRASSTVTSHAALISMLHRNAGLDVPNASPLVFRTMKKINRVAVINGERAGQAVPFRLRDLLMV |
| | DRHWSGSENLQSLRDLAFLHVAYATLLRISELSRLRVRDVMRAGDGRIILDVAWTKTIVQTGGLIKALSR |
| | HSTQRLEEWITVSGLASHPDAYLFSAVHRSGRAQITDKPMTTRALEQIFSRAWAIAGKSGAVKANKNRYT |
| | GWSGHSARVGAAQDMADKGYSIARIMQEGTWKKPETLMRYIRHVDAHKGAMVEFMEQIADGDHSGOSS |
| 1252 | WP_115764642.1 |
| | MSELVPLTPLMVDRNSDITERLRQFVQDKEAFSPNTWRQLLSVMRICNRWSEDNQRSFLPMSADDLRDYL |
| | SFLAESGRASSTVTSHAALISMLHRNAGLPVPNVSPLVFRTMKKINRVAVINGERAGQAVPFRLTDLLAL |
| | DKEWAGSENLQSLRDLAFLHVAYATLLRISELSRLRVRDVMRAGDGRIILDVAWTKTIVQTGGLIKALST |
| | RSTQRLEEWIEASGISSQPDAWLFTAVHRSGRPLIAEKPMSTRSLEQIFSRAWRTAGKEGAVKANKNRYT |
| | GWSGHSARVGAAQDMADKGYPIARIMQEGTWKKPETLMRYIRHVDAHKGAMVEFMEQYSDPDYPG |
| 1253 | WP_111138305.1 |
| | MRKSAPLTNLTVTRNSDIAERLRQFVQDKEAFSPNTWRQLISVMRICHQWSEDNQRTFLPMSAEDLRDYL |
| | AFLAESGRASSTVTSHAALISMLHRNAGLAVPNASPLVFRAMKKINRVAVINGERAGQAVPFRLGDLLLL |
| | DQRWSGSDNPQWLRDLAFLHVAYATLLRISELSRLRVRDVMRAADGRIILDVAWTKTVVQTGGLIKALSS |
| | RSTQRLEEWMEVSGLAAHPDAYLFCAVHRSGRAQIMEKPMSTRALEQIFSRAWDIAGKCGAIKANKNRYT |
| | GWSGHSARVGAAQDMADKGYPIARIMQEGTWKKPETLMRYIRHVDAHKGAMVEFMEQIADSDVPG |
| 1254 | WP_008839747.1 |
| | MQDARKTDDTADDDLPDIVDLVVEMGHVAGSPARVDTLVEAATGFAKPARSENTQAAYAKDWRHFTGWCR |
| | REGFDPLPPSSQVIGLYIGACAAGDPKHGAPALSVATIERRLSGLAWNFAHRGQPMDRVDGHIATVLAGV |
| | RKKHAKAPRQKEPLLGDDLLAMIAMLGQDLRGMRDRAILLLGFAGSLRRSEIVGLDVVRNENGDGAGWVE |
| | IYPDKGALVTLRDRTGWREVEVGRGSSDQSCPVVALETWIKFGRIARGPLFRRISKDNKTVYVERLSDKH |
| | VARLVKKTALAAGIRADLAEGEREQLFAGHSLRAGLASSAEIEVRVQEQWGHASAGMTQKYQRRRDRFRV |
| | NRTKASGL |
| 1255 | WP_065417888.1 |
| | METVNGVLKYAQKSKLIYNLPTDIEKQPMNKPKVEFWAKEEIDFYLDKIHDSYLYTPILIEIFTGLRVGE |
| | LCGLRWCDIDFEDRYLTVNNQVIYDRELKMLVFSKILKTDTSHRKITMPKILTDYLKSIKSDALDTDFVV |
| | LDREGSMCNPRNLSMNFTKSIHKYKKSIDDLKIEDRSIPENYMQLKQITFHALRHTHATLLIFNGENIKV |
| | ISERLGHKNISTTLDTYTHVMEDMKNSTADLLDNIFRYIPSTT |
| 1256 | WP_058413992.1 |
| | MSDLDRYLNAATRDNTRRSYRAAIEHFEVSWGGFLPATSDSVARYLVAHAGVLAVNTLKLRLSALAQWHT |
| | SQGFPDPTKAPVVRKVLKGIRAVHPAREKQAEPLQLKHLEQVVGFLQEDANAAREAYDQPRLLRAKRDTA |
| | LILLGFWRGFRSDELCRLAIEHVQATPGAGISLYLPRSKSDRENIGKTYQTPALLRLCPVQAYSEWLSAS |
| | ALVRGPVFRAVDRWGNLGEEGLHPNSVIPLLRQALERAGIPADQYTSHSLRRGFASWAHRSGWDLKSLMS |
| | YVGWSDIKSAMRYVEAAPFLGMTLATPALV |
| 1257 | WP_099235164.1 |
| | MSDLDRYLNAATRDNTRRSYRAAIEHFEVSWGGFLPATSDSVARYLVAHAGVLAVNTLKLRLSALAQWHT |
| | SQGFPDPTKAPVVRKVLKGIRAVHPAREKQAEPLQLKHLEQVVGFLQEDANAAREADDQPRLLRAKRDTA |
| | LILLGFWRGFRSDELCRLAIEHVQATPGAGISLYLPRSKSDRENLGKTYQTPALLRLCPVQAYSEWLSAS |
| | ALVRGPVFRAVDRWGNLGEEGLHPNSVIPLLRQALERAGIPADQYTSHSLRRGFASWAHRSGWDLKSLMS |
| | YVGWSDIKSAMRYVEAAPFLGMTLATPALI |
| 1258 | WP_003139553.1 |
| | MASARYRQRGKKKLWLVEIRQGDKTLDSKSGFRTKKDAQKYAEPILQKIRNGNTLRPDMTLVDLYQEWLD |
| | LKIIPSSRQQTTINKFILRKKIIKKYFGNKKVSEIKPSDYQKAMNEYGNHINRNGLGRLNNDIHNAISMA |
| | IADKVLIDDFTINVELYSTKVAQAVDDKYLQSEADYNAVIEFITQKLDYHKSVVPYVIYFLFRTGMTYAE |
| | LIAVTWKDIDFTKSVLKTYRRYNTGTHKFVPPKNKTSIRTVPIDAKSLIILKSLQSQQKKANQELGVDNN |
| | ENFIFQHHSLRYDIPLIETVSKAIKEMLKTLKITPLLSTKGARHTYGSVLLHRGIDMGVIAKLLGHKDIS |
| | MLIEVYGHTLQERVEEEYQEVRNVLK |
| 1259 | WP_132898417.1 |
| | MSDDLDDTALTRISSTPLIPLLLDEEIEAARAYVAAARAPATRRAYESDWRIFLAWCAAHAIDPLPAAPG |
| | AVAIFLSGEAQEGARPSTIGRRLAAIGYMHAQAGLDPPQQQAGAIAIRNVVAGIRRTHGVKKVQKRAADG |
| | DMLRDMLRACDGDSIRDVRDRALLAIGMAAALRRSELVALNIDDVAITPDGLLITIRKSKTDQEGEGATI |
| | AVPEGRRIRPKALLLAWIACAGFGDGPVFRKLTPQGRITAKPMSDRGVALVVKARASGAGYDSAHVAGHS |
| | LRAGFLTEAARQGATVFKMKEVSRHKSLEILSDYVRNHELFRDHAGERFL |
| 1260 | WP_120809906.1 |
| | MEKIAHYLAAATRDNTRRSYAAAIRHFEVEWGGFLPATADSMARYLADHAETLSVSTLKQRLAALAQWHQ |
| | QQGFPDPTKAPVVRQVLKGIRALHPAQQKQALPLQIRQLEQLLAWLDGAIELAIQQQDHAARLRCRRDKA |
| | LLLLGFWRGFRGDELLRLQIENIALVAGEGMNCYLAQSKGDRQLQGRVFRVPQLSRLCPVSAYGEWLADS |
| | GLREGAVFRGISRWGVIGEDGLHINSLIPLLRRLFAAAGLAEAARFSGHSFRRGFANWASANGWDLKTLM |
| | AYVGWKDIQSAMRYIDAADPFARQRIENSLPPAPALPPVAD |
| 1261 | WP_075758185.1 |
| | MAKRANGEGTICKRKDGLWTGAVTIGRDAETGKLIRKYFYGKSKTEVQEKKAAQLEKTKGLAYLDADKLS |
| | VSQWLNKWLTLYARTTVRQNTLEGYQFIVDNHVIPALGAVKLGKLQSNQIQGMVNAILDKGGSPRLAEFS |
| | FAVLRRSLRQALKEELIYRDPTLAVSLPKKQKKEIVPLTDEEWTALLATAAKPVFRSLYAALLLEWGTGI |
| | RRSELLGLRWPDIDFARGAVSICHAAISTKDGPQLAEPKSKKSRRTLPVPPTVLAELKKHKSRQAARQLK |
| | AKTWENNNLVFPTRSGGLQDPRVFSRRFARLVKAAGITSGLTFHGLRHDHATRLFAQGEHPRDVQDRLGH |
| | ASITLTMDTYTHSMPSRQQAIASRLEANLPGRKPQADTAAAETAATAPTAAAVQQPVLQ |
| 1262 | WP_063313927.1 |
| | MVSKADRYLEASVRQNTSKSYAAALSHFEVTWGGFLPTTTESVVRYIAEYADQLALSTLKQRLAALANWH |
| | QSNGFPDPTKAPKVRQLLKGIRAVHPVQQKQAAPLALLHLEKAVAHLEDEVVQAKAAGNMGALLKATRDI |
| | ALLTIGFWRGFRGDELARLTIENTHAERYVGIRFYLGSSKGDRHNTGREYKTPSLSKLCPVEAYLNWIEA |
| | AGLTRGGIFRGIDRWGNISDRPLAAHSLVPLLRDTLNRCGLPSEIYSAHSIRRGFATWAASSGWDIKTLM |
| | EYVGWSDMKSALRYVEPAQQFGGLIRKLEG |
| 1263 | WP_038202623.1 |
| | MPIYKRSNKYWIDVSAPNGERIRRSTGTEDKLKAQEYHDKVKHELWQLERLDKQPERYFEEMIIMALRDA |
| | EPQSCFANKQIYARYFLSIFKGRKISSITSEEITNSLPTHSNETKSKLSNATQNRYRAFIMRSFSLAYKM |
| | GWITKPHHVTRLREAKVRVRWLERHQAVELINNLSLDWMKKLVSFALLTGARKGEIFSLIWRNVNLDRRI |
| | AVITAENAKSGKARAVPLNDEVVSILRNLPRECEFVFSSNAKRIKQISRTDFDRALKKSGIDDFRFHDLR |
| | HTWASWHAQSGTPLMALKEMGGWETLEMVNKYAHLSGEHLAKYSGVVTFLTQTDKCSSQKQHLKLLTG |
| 1264 | WP_110560945.1 |
| | MLSDVRILGTSRQAQAALHARVDPLTQQRLAETQDPARWREILSTARFTPPLPLLLAGIELPDGSYSPDT |
| | PLAQDVPYASAAQQMAQDHVADIPSGFELAIGLEIDDGTPCFLAWFRPLQPVGSCSGTVDAAPPAPVGQP |
| | AAAVAQWFSVVSAQPVPEHDGRLATARQAADAYMHRSKAENTLRTYRAAVRSWCRWAAGHALPALPARSE |
| | DVAAYLADMALQGRRTSTIDLHRAALRYLHHLAQTAVPTAHPMVTATLAGIRREAKETLPRQKTALTWDR |
| | LVRVVEAISPHDLVGARDRAILLLGFAGAFRRSELAALKVEDITVDEDGMQIRLGRSKGDPQRKGALIGI |
| | PRGLTRNCPVRAYETWLRQAGITEGPVFRRIWSARDRRAGATPVGTPPRIGPHALSDRAVTDIIRKRCGD |
| | THLEGDFGGHSLRRGAITTGAKDGYDLLELKRFSRHKSLQVVETYIDEASIKARHPGRSRF |
| 1265 | WP_102325737.1 |
| | MLSDVRILGASKRAQAALHDRVDPLTRQRLAETQDPARWREILSTARFTPPLPLLLAGIELPDGTYSPDT |
| | PLAQNVPYASAAQQMAQDHVADIPSGFELAVGLEIDDGTPCFLAWFRPLQPVEPCPGMADAAPPPAPVGQ |
| | PAAAVAQWFSVVSAQPVPEHDGRLATARQAADAYMHRSKAENTLRTYRAAVRSWCRWAAGHALPALPARS |
| | EDVAAYLADMALQGRRTSTIDLHRAALRYLHHLAQIAVPTAHPMVTATLAGIRREAKETLPRQKTALTWD |
| | RLVRVVEAISPHDLVGARDRAILLLGFAGAFRRSELAALKVDDITVDEDGMQIRLGRSKGDPQRKGTLIG |
| | IPRGLTRNCPVLAYETWLRQAGITEGPVFRRIWSARGHRAGATPVGTSPRIGPHALSDRAVTDIIRKRCG |
| | DTHLEGDFGGHSLRRGAITTGAKDGYDLLELKRFSRHKSLQVVETYIDEASIKARHPGRSRF |
| 1266 | WP_110095979.1 |
| | MLSDVRILGSSRRAQAALHARVDPLTRQRLAETQDPARWREILSTACFTPPLPLLLAGIELPDGSYSPDT |
| | PLAQGVPYASAAQQMAQDHVADIPSGFELAVGLEIDDGVPSFLAWFRPLQSVGSRSETADAAPPAPVGQP |
| | AAAVAQWFSVVSAQPLPEHDGRLATARQAADAYMHRSKAENTLRTYRAAVRSWCRWAAGHALPALPARSE |
| | DVAAYLADMALQGRRTSTIDLHRAALRYLHHLAQIAVPTAHPMVTATLAGIRREAKETLPRQKTALTWDR |
| | LVRVVEAISSHDLVGARDRAILLLGFAGAFRRSELAALKVDDITVDEDGMQIRLGRSKGDPQRKGTLIGI |
| | PRGLTRNCPVLAYETWLRQAGITEGPVFRRIWSARDRRAGATPVGAPPRIGPHALSDRAVTDIIRRRCGD |
| | THLEGDFGGHSLRRGAITTGAKDGYDLLELKRFSRHKSLQVVETYIDAACIKARHPGRSRF |
| 1267 | WP_014106907.1 |
| | MLSDVRILGTSRRAQAALHARVDPLTQQRLAETQDPARWREILSTARFTPPLPLLLAGIELPDGTYSPDT |
| | PLAQNVPYASAAQQMAQDHVADIPSGFELAVGLEIDDGMPSFLAWFRPLQSVGACPGTADAAPPAPVGQP |
| | AAAVAQWFSVVSAQPVPEHDGRLATARQAADAYMHRSKAENTLRTYRAAVRSWCRWAASHALPALPARSE |
| | DVAAYLADMALQGRRTSTIDLHRAALRYLHHLAQIAVPTAHPMVTATLAGIRREAKEVLPRQKTALTWDR |
| | LVRVVEAISSHDLVGARDRVILLLGFAGAFRRSELAALKVDDITVDEDGMQIRLGRSKGDPQRKGTLIGI |
| | PRGLTRNCPVLAYETWLRQAGITEGPVFRRIWSARGYRAGATPVGTPPRIGPHALSDRAVTDIIRKRCGD |
| | THLEGDFGGHSLRRGAITTGAKDGYDLLELKRFSRHKSLQVVETYIDAASIKARHPGRSRF |
| 1268 | WP_070406227.1 |
| | MLSDVRVLGSAVHARRALLKRVDPRTQARLDGVDPLAAAPILSSARFTPPLPLLLAGHALADGNETPDYM |
| | IGAAFPDAATAEQAARRHLGDAPSGFDVAVGLEIEVDAPRFVAWLRRQERVSVHASDPPSLPPAPVGQAP |
| | ATVARWFALVSSQPVPQPDGTLRTARQAVEAYVQRSKAVNTLRSYRAAVRSWCQWASAHDLPALPARSED |
| | VAAYLADMALRQRKTRTLDLHRAALRYLHHLAHITVPTSHPLVSATLAGIRREADHPAPLQKTALTWEKL |
| | TQAIDAMEGDDLVALRDRAILLLGFAGAFRRSELAGLAIQDIAIDEEGLQIRLTRSKGDPSAKGVFIGIP |
| | RGITRHCPVRAYEAWLRASCLTEGPVFRRVWRSRLPTPGVVPPRSKIGAAALSDRSVAEIVRQRCGGAGL |
| | EGDFSGHSLRRGAISTGAQDGYDLLELKRFSRHKSLQVVETYVDAASVKKRHPGRSRF |
| 1269 | WP_039683693.1 |
| | MTEGALVLASRWSNAANRRREGLRAAHEQNADALTDLLVTYMRLKSSRGARVSQLTLDHYCESVRRFLAF |
| | TGPPESPERALNQLAAEDFEVWMLTMQQASLSASSIKRHLYGVRNLMKALVWAGALASDPSAGVRPPSDT |
| | TPAHAKKQALSVARYAELLALPASMHPGDTLRAHRDTLLLELGGSLGLRAAELVGLNATDIDLNERQLRV |
| | LGKGSKGRTVPMTARVERSLRLWLMSRSSLQALNKLETPALLVSLSGRNYGGRLTTKGARTIAATYYQEL |
| | GLAPELWGLHTLRRTAGTHLYRATRDLHVVADVLGHASVNTSAIYAKMDTEVRREAMEAMERLRDSND |
| 1270 | WP_058101978.1 |
| | MARRKTPTVEYTINGVTRERKKRTETFGTLEMLKSGKWRVKYYLNGHRYATSAFDDKMEAERYRAELEAE |
| | RRAGTLKPPAAIKATNFKEYAHTWIEQHRTSKGKPLAPRTKAEILRMLEHGLSYFDPYSLTVIDAPLIRK |
| | WHAKRCKDAGATTAGNEARVLKAILQTAVNDDVLEKNPVPGELTRSKTGKEHRAPTTGELKRILDHLEGQ |
| | WRVAVLIAAFGGLRAGELSALERQDIEVRNGRVVIHVTKQAQWLDGEWIVKPPKSVDGVRFVTLPEWITP |
| | DVETHLRRNVSQFPNCRVFVTSRGAKYVSTATWGRVLHKAMADAGIDAPIHWHDLRHFFGTNLAKSGVGI |
| | KELQAALGHGTPAASLSYLEQEHGLTAELANRLPRLDDSSSLIVFPRKATA |
| 1271 | WP_073288322.1 |
| | MSTEITRIPDEPQALGSQLSTAAANVARYIKAGLEGADNTVLAYSADLKSFGDFCQLHGLNQLPADVATL |
| | ARYVADLADIPRKLSTIRRHLAAIHKHHQLRGYLSPVRADELALVMEGITRTLGKRQKQAPAFTVEELKE |
| | SIRRLDVTTTAGLRDRALLLLGFAGAFRRSELVALDVEHLEFTEKALIVHLAKSKTNQAGEVEDKAVFYA |
| | ATSAFCPVRCTRAWLQQLGRNTGPLFVSLKRGKVKGQAMPTLKRLSPLRVNELVQLHLNHDEDGHKVPEK |
| | NYSAHSLRVSFITISVLRGQSNRFIKNQTKQKTDAMIDRYSRLDDVVSFNAAQNLGL |
| 1272 | WP_102906331.1 |
| | MQPDSLPAVLSVHPVLDPARLSRLTEESARELIRQGQSANTRASYQGAMRYWAAWFAARYGQELKLPMPV |
| | PVVVQFIVDHAERELVLEDADEAAAPAGKKTRRKVAKKVPLVFDLPPEVDQVLVAHGYKKKLGAYAQNTL |
| | VHRLAVLSKAHQNVNVDNPCNHTQVRELIKNVRSGNAKRGVKPHKQAALTKAPMDALLATCDDSPRGKRD |
| | RALLLFAWASGGRRRSEVADAIMENLRKVDSRGYLYKLGHSKTNQDGKENPDDAKPVSGKAAAAMDAWLE |
| | VSGITEGPIFRRILKGGKVLDEPLDPTAVRKIVKRRCLQAGLPGDFSAHSLRSGFVTEAGRRKMDPADAM |
| | AMTGHRHYETFMGYYRAEDPLDRKASRMLDGDDAAVE |
| 1273 | WP_045572321.1 |
| | MTYLVYSSDVFKETELRKLDDGTFHCQPTNDNIGSLPTLFYQNGIFNYEANSYLFYLKAIKKAEDLSPCA |
| | QALRAYYQFLEDNGLNWDNFPPVKRLKPTYLFRSHLLKQIKQGELAHSTASVRMNQIVNYYKWLMHDGYL |
| | CIKNEKEAPFKMEFVSIQNNGTLAHISPTFTIETSDLRIKVPRDADSKNIRPLSPLSIDALSVLTHHLLR |
| | TSEELRLQSLLAIDTGMRIEEVATFTLDALDTAIPLAESQYRFEMLLCPRSTGVQTKFLKTRTVEISSNL |
| | LQLLNQYRVSERRLKRVAKLNEKIEQLDNEVPPFTQKKIELLDRSKRHEPLFISQQGNPVTGKIIESRWV |
| | EFRAEIRQAEPSFSHRFHDLRATYGTYRLNDLLEANLPVVECMELLMGWMGHKNESTTWKYLRFLKRKEA |
| | FKVKFGILDSIMHEALGGEDE |
| 1274 | WP_041338471.1 |
| | MTSKARFPGYPLFDTAELIHEQADLELYPGLQAALMALPQSHRDDFHIAQRFLVKYSDVSGTYNRFRSEI |
| | QRFLNYTWHIAKRHLSQADSDLLSSYFSFLKTPPASWVSRGIYPAFFDSNDQRHQNPDWRPMAQRSKDSN |
| | APYSVTQASLNASRTALQTFFKYLMAQDYLQRNPLLDVRKRDRNAKPSLDKDADAEVRRLTDWQWSYLLE |
| | TLTQLASANPKCERNLFVIVTMKSLFLRVSELAPRPVDRGQMRTPSFSDFRRTIVDGEAYWIYSIFGKGD |
| | KTRQVTLPDAYLSYLKRWRLHLGLTSPLPVPGESTPILPSAKGDAIGKRQVQRIYEQSIVATADRMEQEG |
| | YGDEARQLLAIRTETHYLRHTGASQAIEAGGDIRHISEELGHANATFTESVYVNSEQARRRTEGRRRLV |
| 1275 | WP_011043709.1 |
| | MARKVKPLTNTEVKQAKPKDKIYKLSDGDGLQLRIMPNGSKQWLLDYFKPYTKKRTSFSLGSYPDVTLAN |
| | ARAKRASSRELLAQDIDPKEHKEDHHREQLLIASHTLKSVAEDWFAIKKTTITEVTAKSLWRKFENHVFP |
| | KLGHRPIDKILAPEAIEALKPLAAKGNLETTGKIIGHLNNIMTHAVNTGILHHNPLSGIRSAFSAPKVTN |
| | MPTIKPNELGKLMKVISYASIKLVTRCLIEWQLHTMTRPSESAKAEWSEIDLENRLWVIPAERMKMRLEH |
| | KVPLTKQSIEILERLKPITGHRTHLFPSHINHHKHCNVETANKALIRMGYKNRLVAHGLRALASTTLNEQ |
| | EFNADVIESALSHVDKNEVRRAYNRAEYLDSRRELMCWWSEHIEQAVSGNLPVSTLKEQKIICNE |
| 1276 | WP_041736950.1 |
| | MLLTKPVPLYPPYIDLCDFDFDDYPQLDKIFSSNEPWWLEQFNWGKIFLTYIGRNKSAHTYERFRNDVER |
| | FLLWSFIVKKKPIDQLRKSDLLEYADFCWQPPVDWIGTSNQERFKITNGYSAANELWFPYKIQAPKSLKS |
| | QFVIDKKKYRPSQQTLSSMFTAIIVFYNYLMAEDFCIGNPAQIAKKDCRHFIIDSQVKEIKRLTGSQWQF |
| | VLDTAVEMADENAMFERNLFVIASLKTLFLRISELSERPNWSPTMGHFWQDDDENWWLKIFGKSRKLRDI |
| | TVPIDFLPFLERYRASRGLLGLPSSNENSILVEKVRGQGGMTSRHLRRLVQSVFDQAHENMRRSEGENKA |
| | LKLKEASAHWLRHTGASMEIERGRPLKDISEDLGHASMATTDTVYVOSENKKRAESGKRRKVD |
| 1277 | WP_070374986.1 |
| | MPIKSKITVTNIKNLVPSDKRLNDTDISGFHARITPLGLITYYLFYRLNGKQVNYRLGVDGQMTPAQARD |
| | LAKSKIADVTQGVDVQALRKQERTSTKYSKLSSLQYFLDEKYTPWLKSRNPKTAEKTVKAFKSSFPKLMD |
| | FQLSDINAWEIEKWRNKRLADGVKPATTNRQINTIKGCLSRAVEWGVIDSHDLRNVKTLTVDNSKVRYLS |
| | KDEESRLRESLKSCDTAFLEVIVLLAMNTGMRKGELLSLQWHDINFDNKILTVDFQNAKSGNTRHLPLNT |
| | EAFNQLIHWQKLSGSEGYVFKGRNNEPLKDFPSLWAEILDEANITHFRFHDLRHHFASKLVMASVDLNTV |
| | RELLGHSDLKMTLRYAHLAPEHKAAAVNLIG |
| 1278 | WP_033082129.1 |
| | MSLTKPIPLYPPYIDLCDFVLEDYPQLEKIFSSNEPWWLEQFNWGKLFLTYIGRNKSNHTYDRFRNDVER |
| | FLLWSFIEKKKPIDQLRKSDLLEYADFCWQPPVTWIGTSNQERFKITNGYSAANEFWFPFKIQAPKSLKS |
| | QYIIDKKKYRPSQQTLSSMFTALIVFYNHLMAEDFCIGNPAQIAKKDCRHFIIDSQVKEIKRLTASQWQY |
| | VLDTAVEMADGDPVFERSLFVIASLKTLFLRISELSERPTWSPTMGHFWQDDDENWWLKIFGKSRKIRDI |
| | TVPIDFLPFLERYRGSRGLLGLPARNENSVLVEKVRGQGGMTSRHLRRIVQSVFDLAHDNMRRSEGENRA |
| | LKLKEASAHWLRHTGASMEIERGRPLKDISEDLGHASMATTDTVYVOSENKKRAESGKRRKVD |
| 1279 | WP_057180966.1 |
| | MKLTELSLADLNVVVPSKHQEAANKYFTDIFNLLPANTQRSYKSDLKQYYDFCFANDMPGLTPDMDLTET |
| | SIKAYVLAMCESQLAHNTIRHRMATLSKFMAIAKFPNPLKNSEYLRDFIKLQMKAHDIYARANQAPALRL |
| | RDLEEINTHVIPKTLLDFRDLAMINIMFDGLLRADEVAPVQLKHIDYKQNKLLVPTSKTDQSGKGSLRYI |
| | SNTSISYVTAYIAEANIDRKSKREKVKDDPTRINKGILFRGISPKGTTMLPFDETVTRLAHMQKIAYVNI |
| | YKSLKRIAKKAGIDLPITCHSPRVGAAVTMAENGVSMKKIQDAGDWKSPDMPARYTEQADIGNGMSDIAN |
| | IFKR |
| 1280 | WP_051743915.1 |
| | MASEAPDPDGTLPATVPQSALPDILRADLERAAAYKKAARSSATHRAYGSDWTIYTDWCAARGLAPMPAH |
| | PEQIAAFVANQADAGFKPTTIERRVAAIGHYHRASNYPAPTAHPEAGGLREALAGIRNDKRVKKVRKNAA |
| | DASALRHMLAEIKGASLRALRDRAILAIGMAAALRRSELVALTLQSVGILEHGLELYLGATKTDQAGEGA |
| | TIAIPEGTRIRPKSLLLDWITAVRALEADVERAPADEAAMPLFRRLTRSDQLTGEPMSDKAVARLVKRYA |
| | ASAGYDASKFSGHSLRAGFLTEAASQGATIFKMQEVSRHKTVQILSEYVRSADRFRDHAGDKFL |
| 1281 | WP072598906.1 |
| | MASDDPSDTGNLPVTVPQPALPDILRAEVDRAADYAKASRSAATQRAYASDWDIFTAWCDVRGMESLPAT |
| | PAAVATFLASEADSGLKVPTIGRRLAAIGYHHRQAGFDPPQEMAGASAIKEVLAGIRREVGTRPERKAPA |
| | DADALRDMIRTIEGDDLRAVRDRAMLAIGMAAALRRSELAGLLIDDVELPPEGLRLLIGRSKTDQSGEGA |
| | VIAIPEGRRIRPKALLLAWIDAAMEAARNLNNPLITFESGPLFRRLTRGGELTADPVSDRAVARLVQRCA |
| | AAAGFDPTDYAGHSLRSGFLTEAARQGASIFKMRDVSRHKSVQVLADYVRDFEMFRDHAGEKFL |
| 1282 | WP_069337675.1 |
| | MASDDPSGSDNLPATVLQPTLPDILRAEVERAATYAKASRSPATQRAYASDWEIFTAWCDARGLASLPTT |
| | PAIVATFLAFEADRGIKANTIGRRLAAIGYHHRQADVDPPQEQSGAGAMLEVLAGIRNALGTRKDRKTPA |
| | HADALGAMLATIIGNDLRALRDRAVLAIGMAAALRRSELVALWIEDVELPTEGLRLWIGRSKTDQTGEGA |
| | VIAIPEGRRIRPKALLLAWTEAAMAGARELNNPLITFETGPLFRRLTRGGELTADPMSDRAVARLVQRCA |
| | ANAGFNPAEFAGHSLRSGFLTEAARQGASIFKMRDVSRHKSVQVLSDYVRDAELFRDHAGEKFL |
| 1283 | WP_060734294.1 |
| | MVPRPDMVVASPELDGRSGSNRAVRRSLLTAETDREAIDAWVSSYDSPNTRETYRREAYRLWLWAVLECR |
| | KAFSSLGHEDLLEYRGFLLDPQPAHLWVSEGGQKFPRADPRWRPFYRKLNKAGQQQAMTILNVLFSWLVE |
| | SRYLEGNPLSLSRRRKKPTEPQVHRHLSPEMWRQTLEYVEELPRGTSREQRHYHRARWLVSLFYLTGARI |
| | SEVVSTSMGQFYAAQGEDGEIRWWLRIQGKGEKARDVPATSDLMAELAVYRESYGLSPIPHRDEVIPLMM |
| | RYGERMLPMTRSSAHVAIKQVFKGAAVRLRAKGPEWKNRADLLEAASAHWFRHTAGSHMASKMNLVTVRD |
| | NLGHGNISTTNTYLHTGNDARHQETEQHFKIEWPRPVK |
| 1284 | WP_036365362.1 |
| | MLTDTAIKRLKPSTDCTPNKPDKYSDGNGLQLIVRPTGTKVWLVAYRYHGRQTNITLGRYPTISLQQARL |
| | QALEIKQKLAQGIDPKTAKPNTVLFGDIANEYHTQRDRNNPINKGKYTVSKVTHKKDLSQYNNDIAPHIA |
| | HLDINAVTPVMILDIAKRIEKRGAYDMAKRAIRQIGAIFRHARDKGLYDRLPPTDGLEKRLTKRKQEHFA |
| | RLEFHELPQFFSHVHHSTCEPLTKLAFKFICLTFVRTIEMRFMQWAEIDWDNYLWRIPPERMKMDKPHIV |
| | PLAPQAIEILHQIKAMGLSDEFVFYNPKTKKPVSENFLTQALKRLGYQGRMTGHGFRGIASTKLHELQYN |
| | HECIELQLAHAKADKVSMAYNGAEHLPYRVQMMKEWAKLIEHACQ |
| 1285 | WP_088652586.1 |
| | MPSEAEKSTSAPSGDFEDARIDDRDHDERGDIALPAHVAGTGTLDRLVNTARDYARVASSENTLKAYATD |
| | WTHFTRWCRMKGAEPLPPSPEIVALYLADLASGSGPSPALAVSTIDRRLSGLAWNYAQRGFILDRKNRHI |
| | ATVLAGIKRKHARPSVQKEAILAEDILAMVATLTYDLRGLRDRAILLLGYAGGLRRSELVSLDVHKDDTP |
| | DSGGWVEIMEKGALLTLNAKTGWREVEIGRGSKDQTCPVHALEQWLHFAKIDFGPVFVGTSRDGKRASKT |
| | RLNDKHVARLIKRTVLDAGIRSELPEKDRLALFSGHSLRAGLASSAEVDERYVQKHLGHASAEMTRRYQR |
| | RRDRFRVNLTKAAGL |
| 1286 | PLX79396.1 |
| | MTADSDPVLLSFKCYLRDERNLSPHTRSAYMRDLLEFRQVITSLSGRENGFDWVAVDHLTIRRYLAYLHK |
| | RNRRTTIARKLSALRTCFRFLVREGVVQSNPADLVATPRRETFLPQTMTIDEVFALLEGKGLGESSRLRD |
| | KAIFELLYSSGLRIGELTSLDIGRVDMEQRLVRVVGKGSKERIVPIGSKAREALVAYLEARSWPAEKEPL |
| | FLNFRGGRLSARSVQRHLKQLLLAAGLSTELTPHSLRHSFATHLLDGGADLRAIQELLGHSSLSTTQRYT |
| | HVSMEQLTAVYDKAHPRSRKK |
| 1287 | WP_012852732.1 |
| | MDGPTLQDLAERWLDHKRASGRGMSDNTEAAYRADLNAWGRALADHHAIDTPDQTRPLEALHTGHLTAEA |
| | LTAAAASFYREGKTAATRSRRISALRGWCAWLVRTGHLTADPTTDLETPRLPRRLPVALTDAQLAAIVQA |
| | ASTPWQGARAQWVRLDRALLALFAGAGARTGEVVALRVGDVICEEDGGGLLRLRGKGGAHRNVPLHADAM |
| | QPVTDYLDERRALLGPFDAEDPLLVARNGKAITTGMIEYRVDQWFRRAAVRRPEGELAHVFRHTYAVGVL |
| | QNGASLNELQAVLGHQNLATTSIYTKVAAEGLKDVARVAPVLRHLRATRPAPTSAPPG |
| 1288 | WP_012852733.1 |
| | MRPAEFEPICVQEAVDRYVEMVRAKALTGQFSPATAEVYCRDMAVFAELAGPGRLLDDLDGADVDAVLLA |
| | FARRPDGRRRRHDPPPAGRALQSAASQARFRRSVSVFFRYAATAGWVRLDPMRAVTVMPRQRGGLRAERR |
| | ALTAEQAGGLVQAARRLAECGPAEARTGRAARRDQRTEIRDGLVVLLLATVGPRVSELTGANVEDFFVND |
| | GRWYWRIFGKGGRTRDVPLPEAVARVLQAYLERGRPLLDRGVEPKALLLSWRGRRLARGDVQAVIDRVLA |
| | RVEPSRRRAVTPHGLRHTTATHLLAAATDMDAVRRVLGHADLATLSRYRDELPGELEAAMRVHPLLKDQA |
| | PGG |
| 1289 | WP_065935487.1 |
| | MDVLNITNQISQVDETPLDLHFLTLNAQEAAADFIAAGTAANTVRSYRSALAYWSAWLQLRYGHALGDTH |
| | LPVEVAVQFVVDHLARPTDDGKWVHLLPASIDAALIRAKVKAKPGALAYNTVSHRLSVLGKWHRLNSWDS |
| | PTDAPVLKSLLREARKAQSRQGLSVRKKTAIVIESLQALLATCTDGLRGQRDRALLLLAWSGGGRRRSEV |
| | VNLQISDVRQLDTDTWLYALGVTKTNTGGVRREKPLRGPAAEALSAWLLAAPAESGPLFRRMYKGDKVGS |
| | TGLSADQVARIVQRRAKLAGLKGDWAAHSLRSGFVTEAGRQGVPLGDVMAMTEHRSVSTVMGYFQAGALL |
| | ESRATTLLKFSTVENEDTSGGHHLASDSKNQA |
| 1290 | WP_010452301.1 |
| | MSELDRYLHAATRDNTRRSYQAAIEHFEVGWGGFLPATSDSVARYLAAHAGVLSINTLKLRLSALAQWHN |
| | SQGFADPTKSPVVRQVFKGIRALHPVQEKQAQPLQLQHLEQVIASLDGEVQAALALQDRPRLLRARRDTA |
| | LILLGFWRGFRSDELCRLEVGNVMAQAGAGITLYLPRSKSDRDNLGRRYQTPALQRLCPVQAYIEWINCA |
| | ALVHGPVFRGIDRWGNLGEEGLHANSIIPLLRQALGRAGIAAEHYTSHSLRRGFATWAHRSGWDLKSLMS |
| | YVGWKDLKSAMRYVEASPFEGMSLAVEKPVAQES |
| 1291 | WP_090208726.1 |
| | MGKADLYLKAGARENTRKSYRAAIEHFEMDWGGYLPTTGDGIVRYLANYAGHHSINTLKQRLAALSQWHI |
| | TQGFPDPTKTPDVRRVLKGIRAVHPAKTKQAAPLQLSQLQQVVGWLDTEANGAHHRGDHKCEVRHRRSIA |
| | LVLIGFWRGFRGDELARLEIEHTHAVSGEGISFFLPYTKSDREHQGATYHTPALKMLCPVEAYINWITIA |
| | GLASGPVFRGIDRWGNLSTEGINPHSLIPMLRRILAEAGLPAAMYSSHSLRRGFATWATANGWDIKALMT |
| | YVGWKDMQSALRYIDASASFAGLAVGKRGSELQIGR |
| 1292 | WP_062152119.1 |
| | MATSSTFIVPAIVADTSDDAGERFLEFFAATIRNANTRSAYMRAVEHFLGWRGVAGLASLGDIRPLHIAA |
| | YIEECQGLFSAPTVKLRLAGLRSLFDWLVRTGVMASNPTTSVRGPSHDVQRGKTPILAADEAKRLIASIP |
| | ADTPVGLRDRALIALMTYSFARVSAATGMNVEDLIQTAGRSWVRLHEKRGKVHELPVHHKLLDHLDAYLA |
| | VAGHRDQPKAPLFRSAKGRSGALSNGRLSRHDAYAMVRRRAVAAGIVAKIGNHSFRGTGITTFLLNEGTL |
| | ELAQEMANHSSPRTTKLYDSRRDGITQDAIERIRIE |
| 1293 | WP_013196326.1 |
| | MAALKRATGNDVITDSTITAARSAHVGRHVLIWLEQVKAASLSELDNFGDEGTVEQVMKVWVKLSLLISR |
| | RRPEIAVSSLLKHVLPNIGSQPLKTLNRLRLNRLYNILIADGKKEEARRVFALTKQFLAWAEMQGYLDHS |
| | PIASMKKRDVAGRATPPRSRQLTDAEIWVFWHGLDNWALSEQARWALRLCLVSARRPDEIVQAQKGEFDL |
| | QLGLWMQGTRNKSQREHVLPISPLMRQCIEALLNAADPDSPWLVSAPRDPQQPLSKGALNQALRRMIRAP |
| | RGLGLEPFTPRDLRRTARSKLSALDTPNDVARKIMNHALEGIDRVYDTHDYLSQMRSAMNTFSDAVKQI1 |
| | ECESYHLLRHRYDGETLILSNLSIMAMSR |
| 1294 | WP_013577822.1 |
| | MSKIGSVTTVEGDFAAGNVGQHVLAYLQNVKMTPLAKLDDFDEEGNATVGQVINIWIRLSLILTRRRPEI |
| | AVSSLMKHVLPVIGEVPLNKITRLRLNRLFNVLLADGKVSEAKRVFALCKQFFGWAETQGYLAHSPLSTM |
| | KRRDVGGRNTPPRERTLTDAEIWVFWHSLDLWDISEQCRWALRLCLLTARRPDEVVRARKDEFHLQIGIW |
| | RQGTRNKSARDHNLPLTPLMITCINALLSASPKHSPWLVPSPLDAQRPLSRGAVTQVIRRLLRAERGPGI |
| | DAFTTRDLRRTARSKLSSLNVPNDVARKIMNHSLEGIDRVYDTHDYLPQMKQALEAFSDNIQGIIDAPDY |
| | YDLRHHFEGESLHVRESSLLFMER |
| 1295 | WP_039389914.1 |
| | MTPDLTQIPARSAHVGRHVLIWLEQVKKASLSELDNFGDEGTVEQVMKVWVKLSLLISRRRPEIAISSLL |
| | KHVLPNIGSQPLKTLNRLRLNRLYNILIADGKKEEARRVFALTKQFLAWAEMQGYLDHSPIASMKKRDVA |
| | GRATPPRSRQLTDAEIWVFWHGLDNWALSEQARWALRLCLVSARRPDEIVQAQKAEFDLQLGLWMQGTRN |
| | KSQREHVLPISPLMRLCIEALLRAADPDSPWLVPAPRDPQQPLSKGALNQALRRMIRAPRGLGLEAFTPR |
| | DLRRTARSKLSALDTPNDVARKIMNHALEGIDRVYDTHDYLSQMRSAMTIFSNAVEQIIRCESYHLLRHR |
| | YDGETLTLDDLSVMAMSR |
| 1296 | WP_033768926.1 |
| | MTPDLTQIPARSAHVGRHVLIWLEQVKKASLSELDNFGDEGSVEQVMKVWVKLSLLISRRRPEIAISSLL |
| | KHVLPNIGSQPLKTLNRLRLNRLYNILIADGKKEEARRVFALTKQFLAWAEMQGYLDHSPIASMKKRDVA |
| | GRATPPRSRQLTDAEIWVFWHGLDNWALSEQARWALRLCLVSARRPDEIVQAQKAEFDLQLGLWMQGTRN |
| | KSQREHVLPISPLMRQCIEALLRAADPASPWLVPAPRDPQQPLSKGALNQALRRMNRAPRGLGLEAFTPR |
| | DLRRTARSKLSALDTPNDVARKIMNHALEGIDRVYDTHDYLSQMRSAMTIFSDAVEQIIECESYHLLRHR |
| | YDGETLTLDDLSLMAMSR |
| 1297 | WP_056773790.1 |
| | MTEQISETETDFAAENVGRHVLVYLQQIKATPLAKLDDFDEEGNATVGQVINVWIRLSLILTRRRPEIAV |
| | SSIMKHVLPVIGDVPLNKITRLRLSRLFNVLLAEGKISEAKRVFALCKQFFSWAETQGYLPHSPLGSMKR |
| | RDVGGRNTPPRERTLTDAEIWIFWHGLDLWDISEQCRWALRLCLLTARRPDEVVRARKDEFNLRISVWRQ |
| | GKRNKSARDHSLPLTPLMLVCINALIAASPKNSPWLVPSPKDPGKPLSRGAITQVIRRMLRAERGLGIAP |
| | FTTRDLRRTARSKLSALDVSNDVARKIMNHSLEGIDRVYDTHDYLPQMKQALDAFSDNIHDIINAPDYLS |
| | LRHKFDGEFLQIPQISLLYMEN |
| 1298 | WP_012075809.1 |
| | MNTTLLPLHSGIAPLSVDRLDADARTAAAAFVAAGTAANTVRSYRSALAYWAGWLQLRYHRHLEDSALPE |
| | AVAVQFILDHLARPADGDWVHLLPPEQDAALVDAGVKAKLGALSYNTVRHRLAVLAKWHDLKSWPSPTET |
| | VAVKTLLRDARKAQARQGVSVRKKTAAVREPLEAMLATCTDGVRGLRDRALLLLAWSGGGRRRSEVVGLQ |
| | VGDVRQLDADTWLYALGVTKTETEGMRREKPLRGPAAQALAAWLAVAPAATGPLFRRLYRGGRVGTAGLS |
| | SDQVARIVQRRAKLAGLEGDWAAHSLRSGFVSEAGRQGVPLGEVMTMTEHRSVPTVMGYFQAGTLLGSRA |
| | TRLLALPLEVPDYPEE |
| 1299 | WP_033986789.1 |
| | MNNTIPLLSGDSPLLAVDRLDAEARAAAAAFVAAGTAANTVRSYRSALAYWAGWLQVRYGQTLEMGPLAD |
| | TVAVQFILDHLARPADGDWVHLLPPALDAALVDAGVKAKLGALRYNTVRHRLAVLAKWHDLKSWPSPTDS |
| | AAVKALLREARKAQARQGVSVRKKTAAVREPLEAMLATCSDGVRGLRDRALLLLAWSGGGRRRSEVVGLQ |
| | IGDVRQLDADTWLYSLGVTKTETEGMRREKPLRGPAAQALAAWLAVAPAATGPLFRRLYRGGRVGTAGLS |
| | NDQVARIVQRRAKLAGLEGDWAAHSLRSGFVSEAGRQGVPLGEVMAMTEHRSVPTVMGYFQAGTLLGSRA |
| | TRLLALPLEVPDYPEE |
| 1300 | WP_005752218.1 |
| | MQEQLDKYWNYLRIERQVSPHTLTNYQRQLYRIVDILAENGITSWQAVTPSIVRFILAQSNKDGLKERSL |
| | ALRLSVLRRFFTYLVQQQDINVNPATGVSAPKQNRHLPKNIDAEQVQQLLNNDSKEPIDIRDRAILELLY |
| | SSGLRLSELQSLNLNSINTRVREVRVMGKGNKERIVPFGRYASHAIQQWLKVRILFNPKDEALFVSQLGN |
| | RLTHRAIQQRLEVWGIKQGLSSHLNPHKLRHSFATHMLEASSDLRAVQELLGHSNLSTTQIYTHLNFQHL |
| | AEVYDSAHPRAKRKK |
| 1301 | WP_011271867.1 |
| | MKSYEKAIRQLQKNCSIQYPDEISDSLILQWRKRVVGQSIIEVTWNSYIRQLKTIFKFGIEKQLLPFTKN |
| | PFDGLFIREGKKKRKVYTSSDLKKLSFGITESKHLPSILRPLWFTKTIIMTFRYTAIRRSQLNKLRIKDV |
| | DLLNQVIHIPSEINKNHEYHILPISTTLYPYLKKLLTELSKLNQPVESQLFNINLFSNAVKRKGEKMTND |
| | QVSYIFKVISKYTGIISSPHRFRHTAATNLMKKPENLYIAKQLLGHKDVKVTLSYIEDNIDSIREYTELL |
| 1302 | WP_069481344.1 |
| | MITLKDAWNRYILLLQSLKKSAATMKQYNMDGQHFLSFAHEKNYLYVDHQFQELLLIYCHYLKETYSNIN |
| | TFNHKIATMRGFVDFIFLREWMEPFDYQHILQPRKRQKEALQVLTTKQIGQMANVWPTYFQYAKTVEHAW |
| | LARRNGCIVQVLMETGCKPAELVRMKWSHFQKEKSTLFIANQNGRREVKCSPILMDMLAHYKEETEAMHD |
| | KEVEEWVWVSEASMTKPITTKTVERIFQTMSKDIGKNVRATDLRYTVMQRAFQEEKTLEHIQQEMGYVRK |
| | WVLTERQQRFE |
| 1303 | WP_092837735.1 |
| | MPLPKPGNLPALQPEMLSDATAQAVEELMREGESANTLASYRSALRYWAAWFNLRYGQPITLPVPPAAVL |
| | QFIVDHAQRSSADGLLHELPPAIDAVLVQAGFKGKPGPMALNTLVHRIAVLSKTHQLKEVENPCQDAKIR |
| | DLLAKTRRAYGKRGDLPRKKDALTKDPLMAMLETCDLSTLKGLRDRALLLFAFASGGRRRSEVAGADMKH |
| | LRRHGVSSFTFVLAHSKTNQHAADRPENYKPIAGMAGEALQAWVEAARITEGPVFRRVLKGGRLAGALSP |
| | AAVRDIVKERARAAGLSEDYSAHSLRSGFVTEAASQNVPLADTMAMTGHRSVATVMGYFRSTGSSQAAHL |
| | LDPKAPPDRS |
| 1304 | WP_057202984.1 |
| | MRLPALKTHAALEPGVLSDMTALAVDQLMREGESANTLASYRSAVRYWAAWFNVRYGQPITLPLPPSAVL |
| | QFIVDHAQRTTAEGLAHELPQAIDAVLVDAGFKGKPGPMALSTLVHRVSVLSKAHQVRDMKNPCQDAQVR |
| | ELLSKTRRAYAKRGALPQKKNALTKDPLMAILATCDATTLKGLRDRALLLFAFASGGRRRSEVASAQMRH |
| | LQRSGPTSFVYTLAHSKTNQTGSDRPENHKPIQGMAGEALQAWLEATGITEGPIFRRVRKGGRLGEALSA |
| | AAVRDIVQERARAAGLPDVFSAHSLRSGFVTEAATQKVPMADTMAMTGHRSVASLLGYFRVSDASQAARL |
| | LEEEGPQA |
| 1305 | WP_057267549.1 |
| | MRLPALKTHAALEPGVLSDMTALAVDQLMREGESANTLASYRSALRYWAAWFNVRYGLPITLPLPPSAVL |
| | QFIVDHAQRTTAEGLAHELPQAIDAMLVKAGFKGKLGPMALSTLVHRVSVLSKAHQVRDMKNPCQDAQVR |
| | ELLSKTRRAYAKRGALPQKKNALTKDPLMAILATCDATTLKGLRDRALLLFAFASGGRRRSEVASAQIRH |
| | LRQSGPAAFVYTLAHSKTNQTGSDRPENHKPIQGMAGEALQAWLAATGITEGAIFRRVRKGGRLGEALSA |
| | AAVRDIVQERSRAAGLPDVFSAHSLRSGFVTEAATQKVPMADTMAMTGHRSVASLLGYFRVSDASQAARL |
| | LEEEDPQA |
| 1306 | WP_077019634.1 |
| | MAALTKTPSGTWKATIRRVGWPTVAKTFRTKRDAEDWARRTEDEMVRGVFIQRAPSEKTTVADALDRYER |
| | EIVPTKKASTQRREGARIRELKEHFGKYSLAAVTPDLVGRYRDDRLAQGKANNTVRLELALLGHLFNVAI |
| | KEWHIGLIFNPVSNIRKPRPGEGRNRRLSGREQATLLTAVDEHTNPMLGWIVRLAIETGMRQSEILGLRR |
| | GQVDLERRVVRLTDTKNNDARTVPLTKLAASVLQSALANPVRPIDTDLVFFGEPGRDKKRRAYQFTKVWN |
| | GIKKRTGLVDFRFHDLRHEAVSRLVEAGLSDQEVASISGHKSMQMLRRYTHLRAEELVGKLDALSAAR |
| 1307 | WP_083768887.1 |
| | MIECFWVYFTNRREPLFDGLSSVEEFISHLESERHFSNNTTAAYKNDILQFHDWLQGKDHINSWAAVTSS |
| | DIQDYLLYLKGNQDRAYAPSTQARKMAAIKSFFQFLVAKSVVDQNPASDLISPRVQKYWPKAISVQEVNM |
| | LLAAASDSETPEGIRDRAMLEVLYRTGLRVSELVSLNVDDINLDESHLKCIGRGKTRKVPLSQPAVDVLK |
| | LYLERSRPLLVRGQDEQALFVNHRGQRLTRQGFWLILKAYASEAGIKGITPHTLRHSFAAHMIDGGIDLR |
| | QVQEWLGHASITTTQVYRQIKSNSHSEKIIDIKSREERIPEEVAK |
| 1308 | ACZ42745.1 |
| | MFDGLSSVEEFISHLESERHFSNNTTAAYKNDILQFHDWLQGKDHINSWAAVTSSDIQDYLLYLKGNQDR |
| | AYAPSTQARKMAAIKSFFQFLVAKSVVDQNPASDLISPRVQKYWPKAISVQEVNMLLAAASDSETPEGIR |
| | DRAMLEVLYRTGLRVSELVSLNVDDINLDESHLKCIGRGKTRKVPLSQPAVDVLKLYLERSRPLLVRGQD |
| | EQALFVNHRGQRLTRQGFWLILKAYASEAGIKGITPHTLRHSFAAHMIDGGIDLRQVQEWLGHASITTTQ |
| | VYRQIKSNSHSEKIIDIKSREERIPEEVAK |
| 1309 | WP_059061637.1 |
| | MASIFKRKNKDGTTHWRAVIRVKGYPTVCNHFARKQEADDWAIDVERQIKQGQFNFSKHKNQHTFSELVD |
| | HFINNGALEHHRSAKDSLRHLNYWRERLGNYALVHLTPERLGKERLLLIETPTNRGEKRSSATVNRYMAT |
| | LSSVLSYACRQLRWIDDNPCFNLIKLKENPGRDRVLTQEEVQRLMAACRQSRNGYLYCIVLLAFTTGMRQ |
| | GEILSLTWNQIDFDNKLAHLKETKNGTPRSVPLVEAVIDELR |
| 1310 | WP_056974519.1 |
| | MASIIKRGKSYRVEISNYKHGKNKRISKTFKTKSEAQRWAMQNEIAKGNGVDLALRKDKFSDFYSNWIYL |
| | VKKNDVRSATFLNYTRTIPIVKKLFKNITLGELNDLVVQMKIDEYGETHSRKTTTELLLKIRTSLRYAYG |
| | RGLITSDFAGLIKTRGKELSKRNSALSISDFKKLRSYLLKHHEKDFYILVLLALETGARRGELLGLTNKD |
| | IFKYGISINRSISPSSSDTRLKTKRSKRNISINENVYDILKTVTEKSNGYLFSFDGFQQSAKLARLLKKL |
| | DIPKTTFHGLRDTHASFLFSNDNIRIDYISQRLGHSNLQTTMNYYLELMPEKKHLQDADALSLLDSL |
| 1311 | WP_003330882.1 |
| | MASFRKRGCTCEKKKCTCGAKWEYRIKYVDRQTGKTKEKSKGGFTSKKEAQLAAAEEELKINQFGFAENG |
| | NEVVLNYFSEWLEVFKKPNVKPITYSVQERNVRLNILPRWGKYRLKDITRTEYQKWINELRDHYSEGTVR |
| | RIHSIFSSAIHDAVHEFHIIRENPIQKIKIPKDVENTNRVQYFSKEQLEKFLNSLKTPQKNAKYKHSIQY |
| | YVLFSLMARTGIRIGEALALTWDDFNEKEKSISITKTLVYPLNSTPYISTPKSLKSVRIVKLDEQTVKLL |
| | KKHKINQNEVILRYKNYKASKDNVMFHQHDGRWLRTNVVREYFKEVCKRTDLPVLSPHALRHSHAVHLLE |
| | AGANIKYVSERLGHASTKVTADTYLHITEKIENEALELYSQYIKF |
| 1312 | WP_000876735.1 |
| | MKYNKTKYPNIYYYETAKGKRYYVRRSFFFRGKKREKSKSGLTTLPQARAALVELEQQIQEQELGINTNL |
| | TLDQYWDIYSEKRLSTGRWNDTSYYLNDNLYKNHIKAKFGSTLLKNLDRNEYELFIAEKLQNHTRYTVQT |
| | LNSSFMALLNDAVKNGNLLSNRLKGVFIGQSDIPAANKKVTLKEFKTWIAKAEEIMPKQFYALTYLTIFG |
| | LRRGEVFGLRPMDITQNDSGRAILHLRDSRSNQTLKGKGGLKTKDSERYVCLDDIGTDLIYYLIAEASKI |
| | KRKLGIIKEQHKDYITINEKGGLINPNQLNRNFNLVNEATGLHVTPHMMRHFFTTQSIIAGVPLEQLSQA |
| | LGHTKVYMTDRYNQVEDELAEATTDLFLSHIR |
| 1313 | WP_019821568.1 |
| | MPNKKSSRRKKFERKARNFFNFHYFGLGKNKKEAKGKLRSQSTLFRHVETAAFIQERMGASMLIDITPOM |
| | ALDYLSSRVGKVCSKMLANERRVLERIVYLHEPERRLYIEEKLDPREWVNRAYTHEQIHQIMTHQTPENQ |
| | LATALCFTAGLRVQELLTLQRFDEASASKDRKWRSDLFSGLQGEKYVVKGKGGLYRAVMIPHCLAKKLER |
| | HRLSEPRQIKDRKCTITNRYNITGGKKFTDAFSQLSKRVLGWSHGAHGLRYTYAQDRLNRSIPDKSYEEK |
| | LEIISQELGHFRKEITPHYLHRGTCS |
| 1314 | WP_011239395.1 |
| | MNLDDMLPALASNVAVMDPDALDPLTQQAVDEILAEGTSANTDVSYRTALRYWAAWFALRYRKPLKFPVP |
| | VPAVIQFIVDHAQRSTPGGLRCDLPDSLDEVLVEKGYKAKLGPMALSTLNHRVSVLSSLHKRSPELENPC |
| | RSPAVRDLIARTRRSYAKRGERPKGKAALTRELLEQLVGTCDDSLKGLRDRAILLLGWASGGRRRSEIVS |
| | LRVEDLKRVGPDEFIFELGASKTNQSGTVKADDLKPVVGAAGSALADWLAATGLASGPLFRQIDKSGSLR |
| | GALSASAVRTIVRERCLLAGLDGDFSAHSLRSGFVTEAAKQLIPLGETMALTGHRSIPSVMRYFRAGSVT |
| | TSKAAKLFDEGDKTE |
| 1315 | WP_013695783.1 |
| | MSNIINKLNELEKETNLNLGSSKSLNTLRAYRSDFSDFKNFCSDLNLPYLPTHIKAVSLYMTHLSKSNKY |
| | STLKRRLASINVIHSLKGFHIDTKNPLIKDNLEGIKRKIGIYQNGKKPLLINNLHKIIDVIDYYKIQKYV |
| | RSTRDKAIILIGFSGGFRRSEIVNLKKNDLEFVEEGLKISLRRSKGDQYGEGMIKAIPYFNNKKYCAIIA |
| | LQDWLSARTNNNDLIFPYSDKTVSLILKKYLNIIGLDSRLYSGHSLRSGFATSTASHGADERSIMAMTGH |
| | KSTEMVRRYIKDSNLFKNNALNKLND |
| 1316 | YP_009125517.1 |
| | MASIRSVSRKDGTTFTQVRYRLNGKQTSTSFDDGAHAVEFKRMVEQLGAAKALEVLETTDAASRNFTLAG |
| | WLKHYLDHKTGVEKSTIYDYRKMVEKDITPVLGAIPLAALTAEDVAKWVQGLADKGLAGKTIANKHGFLS |
| | SALNVAASAGHIKANPAVGGAGLVAVPRTERAEMVFLTADQYAKLHDNMPLRWQPLVEFLVASGARWGEV |
| | TALRPSDVNRAEGTVRISRAWKRTYARGGYELGAPKTNKSRRTINVDTAVLDRLDYSGEWLFTNVRGGPV |
| | RGHNFHENHWQPALKKAGLDGLDVKPRIHDLRHTCASWLIAAGVPLPAIQQHLGHESIQVTIGVYGHLDR |
| | SSGRTVAAAIAAALGR |
| 1317 | WP_062041733.1 |
| | MGKTYDVRIWSVRQRKDRGQTSAELRWKTGETPHSQTFRTKTLAEGRRAELLRAAHAGEPFDESTGVPLS |
| | ELRQRNDVSWYQHAREYIEMKWQHSPGSTRRTLAEAMATVTPALVKDTKGMADATTVRTALYSWAFNVSR |
| | RDQDPPDEVAAVLAWFERKSLPTSALADRMQVRAALDTLTRKLDGTTAAASTIRRKRAIFHNALGYAVDA |
| | GRLTDNPLPQVQWKAPEQVAEELDPASVPDPRQALALLDAVRTQSPRGRRLVAFFGCMYYAAARPAEVIG |
| | LRLQDCDLPRRGWGTLQLRETRPRSGSAWTDSGEAHDRRGLKHRPRKAVRTVPIPPDLVNLLRWHVMAYG |
| | VAPDGRLFRTQRGGLIQDTGYGEVWAEARARALTPAQCASLLAKRPYDLRHAAVSTWLSSGVEPQEVAAR |
| | AGHSVAVLFRVYAKCLDGGAATANARIERALKNGS |
| 1318 | WP_044878438.1 |
| | MLLKFAYQDFLDDRRFKNTTEKNIRNYQTMLGAFVEYCIQHEVVSVEDITYNHVRQHLMECQERGNKAGS |
| | INTKIMRIRAFLNYMVECEVITKNPAKRVKMQKEDVKINVFTDEQIRQMLNFYRRIKQRDKSYVAYRDYM |
| | MIVTILGTGIRRGEIISLQWSDIDFVNQTIAVFGKSRRKDTLPITDKLSKELAAYQIFCKQHWGDLSDYV |
| | FVKRDNNQMTENALMLVFKYLGQKMNFKDVRVSAHTFRHTFCHRLAMSGMSAFAIQKLMRHQNIVVTMRY |
| | VAMWGNELREQNDKYNPLNSLNI |
| 1319 | KPU82353.1 |
| | MNKLVVDIKSLELDTLKNLSNAKADNTLRAYKADYRDFLEFCTKHSFKSMPTEPKIVALYLTHLSKYSKF |
| | STLKRRLASISVIHKLKGHYIDTKHPLIMENLLGIKRLRGSNQKAKKPLLINELKTIIDVIDKSKNKFLK |
| | KTRNKSLILLGFAGGFRRSELVSIDYDDIDFVSEGVKIFIKRSKTDQSGEGMIKAIPYFINEQYCPVKNL |
| | KNWINLSDIKTGKVFDISDKSVSLLIKKYAALAGLDEKKYSGHSLRSGFATSTAESGAEERNIMAMTGHK |
| | STQMVRRYIKEANLFKNNALKKLKV |
| 1320 | WP_048499202.1 |
| | MNKKTISQIVEFWKADKKMYVKKSTLSAYILLIENHLIPEFGSNSEIEEEQVQKFVFQKLEQGLSQKTVK |
| | DILIVLKMILKFGAKNKWIQFSPFQIQYPTVRENQQIEVLSRTHQKKVMNFIQEHFTFRNLGIYICLSSG |
| | IRIGEICALTWEDIDTDNGIIHIRKTIQRIYVIENGERRTELLLDSPKTKNSIREIPMSRELLRMLKPFK |
| | KIVNPTFFVLTNDSKPTEPRTYRSYYKNLMRQLEIPEIKFHGLRHSFATRCIESKCDYKTVSVLLGHSNI |
| | STTLNLYVHPNLEQKKKAIDQMFRALK |
| 1321 | YP_195916.1 |
| | MNALVSLDQMMVPAPPDGRKGRNRATSRSQLAAVDDRSAVLAWLARYTDSPATLASYRKEAERLLLWCVL |
| | QRGAALSDLTHEDLLLYQRFLADPQPAERWVMEPGQKPGRNSPRWRPFAGPLWASSLRQALSILNAMFSW |
| | LVEAGHLAGHPLALSRRKRRQAAPRVSRFLPEEHWDVVKAAIEAMPVGSERERLHASRCRWLFSLLYIGG |
| | LRVSEICDARMGGFFSRRGADGRERWWLRNHRQQAARPAWCRPRAILMTELMRYRKAHALSPLPLEGRRH |
| | AIGDDADRPGQAYGTSAIHELVKGVMQAAAAALRRRGSDFGAAAAHLEQASTHWIRHTAGSHLSEKVDLK |
| | VVRDNLGHANISTTSIYLHTEDDARHDATAAGHRVGWRSP |
| 1322 | WP_013397105.1 |
| | MNALVSLDQMMVPAHLDGRKGRNRATSRSQLAAVDDRSAVLAWLARYTDSPATLASYRKEAERLLLWCVL |
| | QRGAALSDLTHEDLLLYQRFLADPQPAERWVMEPGQKPGRNSPRWRPFAGPLGPSSLRQALSILNAMFSW |
| | LVEAGHLAGNPLALSRRKRRQAAPRVSRFLPEEHWDVVKAAIEAMPVGSERERLHASRCRWLFSLLYIGG |
| | LRVSEICDARMGGFFSRRGADGRERWWLEITGKGSKTRLVPATGELMTELMRYRKAHALSPLPLEGEDMP |
| | LVMTLIAPVKPMARSAIHELVKGVMQAAAAALRRRGSDFGAAAAHLEQASTHWIRHTAGSHLSEKVDLKV |
| | VRDNLGHANISTTSIYLHTEDDARHDATAAGHRVGWRSP |
| 1323 | WP_057591291.1 |
| | MNTLVSLDRMMVPIHLDGSRGRNRASSRSQLAAVDDRSAVLAWLARYADSPATLSSYRKEAERLLLWCVL |
| | QRGAALSDLAHEDLLLYQRFLGDPQPAERWVMEPGQKPGRSSSRWRPFAGPLGPSSLRQALSILNAMFSW |
| | LVDAGYLAGNPLALSRRKRRQAAPRVSRFLPEEHWNVVKAAIEAMPVGGERERLHASRCRWLFSLLYIGG |
| | LRVSEICGASMGGFFSRRGSDGRERWWLEITGKGSKTRLVPATGELMSELMRYRKAHALSALPLEGEGTP |
| | LVMTLIAPIKPMARSAIHELVKGVMHAAAAALRQRGSDFEAAATHLEQASTHWIRHTAGSHLSEKVDLKV |
| | VRDNLGHANISTTSIYLHTEDDARHDATAAGHRVGWRSP |
| 1324 | WP_114070645.1 |
| | MKENTVSQNVQATSPNTQLPHVLVGKVADYVRKGLEGSDNTQRAYRSDVYYFIEWCRENGQSEFPATTPT |
| | LSAYVSHLADTHKWASINRKLAAIRKLHELNNVELPTNDRGFKAVMEGIKRTKGIRQKQAPAFQMNELKK |
| | VLRTMETETHAGMRDKSLILLGFAGAYRRSELVDLNIENVEFNEDGAIITLTKSKTNQYGEAEEKAFFYS |
| | PEASLCPIRNLKNWIMRLERTTGPLFVRVRKGDRLTTDRLNDMTVYTTVKKYLGEKYSAHSLRASFITIA |
| | KINGANDSEIMRQSKHKTSLMIQRYTRIEDIKKHNAATKLGL |
| 1325 | WP_120128527.1 |
| | MRRRPRFRGENAMEKADRYLNAGTRENTKKSYRAAIEHFEVTWGGYLPTTGDGIVRYLAEYADQHAISTL |
| | KQRLAALAQWHITQGFPDPTKTPNVRQMIKGIRVIHPARVKQAAPLLLTHLERAINWLENEAAAAQARND |
| | YKVLLRHRRSIAMVLVGFWRGFRGDELTRLTVENTQAYSGEGITFYLPYTKGDRQHEGTTFETPALKTLC |
| | PVEAYLNWITVAGIATGPVFRRIDRWGNLSDKAIQPHSLVPMLRRIFREAGLPEDLYSSHSMRRGFATWA |
| | SANGWDIKALMSYVGWKDMKSALRYVDSSVSFGGLAVRSASARLSNP |
| 1326 | WP_014786680.1 |
| | MTESTEIALWVSQEPETASQAPGLTPAQLQLRQMVLDSVTSPHSRRNYAKALDLLFAFAASRPLTRALLL |
| | EFRTSMEDLAPSTVNVRLAAVRKLVSEARKNGMLSHEDAANLTDIPNVKEKGTRLGNWLTKEQARELLGV |
| | PDRSTLKGKRDYAILALLVGCALRRRELASLTVEDIQMRENRWVIIDLVGKGGRVRTVAIPVWVKKGIDA |
| | WQAAGSIEKGPLLRSVSKGGKIGESLSDWAIWSVVTEAAKEIGIERFGAHDLRRTCAKLCRKAGGDLEQI |
| | KFLLGHSSIQTTERYLGSEQEIAIAVNDSLGL |
| 1327 | WP_065653736.1 |
| | MSNKSIKKIMIAESGAAISTTLSSSSRQFLENTLAQATKRGYAADLKIFFAWAEAHQTAAIPATAETIAN |
| | FLADQASGILSVWLRQESQLINGRPVSVATLRRRLAAIKYAHKLNKIEPSPTDTAEVRETLKGIRRTLGA |
| | KPNAKSALMSQDIQLLIKYIPETITGQRDRAILLLGFAGALRRSELTSLELSDIEVQENGMLVYIRSSKT |
| | DQEQQGQVIGIARSENKANCPVGAIEQWLQSSMILSGPIFRRIFANGKIAITTLSDRTIYNIVKNYCQLA |
| | GLDASRFGAHSLRRGFVTSAAKAKVDPFRIMAVTRHKRLETVKRYVDEANLISDYPGADLLK |
| 1328 | WP_082304040.1 |
| | MSNKSIKKIMIAESGAAISTTLSSSSRQFLENTLAQATKRGYAADLKIFFSLGSEAHQTAAIPATAETIA |
| | NFLADQASGILSVWLRQESQLINGRPVSVATLRRRLAAIKYAHKLNKIEPSPTDTAEVRETLKGIRRTLG |
| | AKPNAKSALMSQDIQLLIKYIPETITGQRDRAILLLGFAGALRRSELTSLELSDIEVQENGMLVYIRSSK |
| | TDQEQQGQVIGIARSENKANCPVGAIEQWLQSSMILSGPIFRRIFANGKIAITTLSDRTIYNIVKNYCQL |
| | AGLDASRFGAHSLRRGFVTSAAKAKVDPFRIMAVTRHKRLETVKRYVDEANLISDYPGADLLK |
| 1329 | WP_076729031.1 |
| | MTLPATLAARARAFADEALSENSRRAYRADWQHYADWCRTHDLEPLPAGPEQVASYLTSMAETHKRATIE |
| | RRLVTIGQAHKLQGLPWVPAHPAVRAALRGMFRRYGRPKKQAAALGVPETLQIVAACEGTVAALRDRALF |
| | LMSFAGAFRRSEIARIRFEDVAFREGAVDVFLPQSKGDQEGEGTIVTVLAGENVATCPVAALRRWLKAAP |
| | TENHIFRAVRADGTVMEAGLHPDSIGRIVQKRAAEAGLVAGPRERISAHGFRAGFITEAYKRGSRDEEIM |
| | SHSRHRDLKTMRGYVRRAKLSDAHPGRNLGL |
| 1330 | WP_012329841.1 |
| | MELDAADPAPGPSRDSFAAPVPFADALPPGLELLIERLEQHARAARGAFADNTLRALAADSRIFAAWCRE |
| | AGRAMLPATPETVAAFIDAQAETKARATVERYRSSVAALHRAAGLQNPCADEIVRLAVKRMNRAKGRRQK |
| | QAEPLNRTSIARMLEVKTPGRLHRRVTEAKREVPLIALRNAALVAVAYDTLLRRSELVSLYIGDLQKGAD |
| | GSGTVLVRRSKADQEGEGAIKYLAPDTVEHIDAWLAAAQLTSGPLFRPLTKGGQVGAGALGAGEVARVFR |
| | EVATAAGLKLARLPSGHSTRVGATQDMFAAGFELLEVMQAGSWKTPAMPARYGERLRAQRGAARKLATLQ |
| | NRA |
| 1331 | KIU27889.1 |
| | MTLPATLAARARAFADEALSENSRRAYRADWQHYADWCRTHDLEPLPAGPEQVASYLTSMAETHKRATIE |
| | RRLVTIGQAHKLQGLPWIPAHPAVRAALRGMFRRYGRPKKQAAALGVPETLQIVAACEGTVAALRDRALF |
| | LMSFAGAFRRSEIARIRFEDVAFREGAVDVFLPQSKGDQEGEGTIVTVLAGENVATCPVAALRRWLKAAP |
| | TENHIFRAVRADGTVMEAGLHPDSIGRIVQKRAAEAGLVAGPRERISAHGFRAGFITEAYRRGSRDEEIM |
| | SHSRHRDLKTMRGYVRRAKLSDAHPGRKLGL |
| 1332 | WP_029361746.1 |
| | MTLPATLAARARAFADEALSENSRRAYRADWQHYADWCRTHDLKPLPAGPEQVASYLTSMAETHKRATIE |
| | RRLVTIGQAHKLQGLPWIPAHPAVRAALRGMFRRYGRPKKQAAALGVPETLQIVAACEGTVAALRDRALF |
| | LMSFAGAFRRSEIARIRFEDVAFREGAVDVFLPQSKGDQEGEGTIVTVLAGENVATCPVAALQRWLKAAP |
| | TENHIFRAVRADGTVMEAGLHPDSIGRIVQKRAAEAGLVAGPRERISAHGFRAGFITEAYKRGSRDEEIM |
| | SHSRHRDLKTMRGYVRRAKLSDAHPGRNLGL |
| 1333 | WP_012329856.1 |
| | MTLPATLAARARAFADEALSENSRRAYRADWQHYADWCRTHDLEPLPAGPEQVASYLTSMAETHKRATIE |
| | RRLVTIGQAHKLQGLPWIPAHPAVRAALRGMFRRYGRPKKQAAALGVPETLQIVAACEGTVAALRDRALF |
| | LMSFAGAFRRSEIARIRFEDVAFREGAVDVFLPQSKGDQEGEGTIVTVLAGENVATCPVAALRRWLKAAP |
| | TENHIFRAVRADGTVMEAGLHPDSIGRIVQKRAAEAGLVAGPRERISAHGFRAGFITEAYRRGSRDEEIM |
| | SHSRHRDLKTMRGYVRRAKLSDAHPGRNLGL |
| 1334 | WP_012010452.1 |
| | MNDQLSDFIHFMTVERGLSENTIVSYKRDLQNYLSFLMTHEQLSDIKDVTRLHIIHYLKQLKEEGKSSKT |
| | SVRHLSSIRSFHQFLLREKVTKDDPSWNIETQKTERKLPKVLSLGEVEKLLDTPNQHTPFDYRDKAMLEL |
| | LYATGIRVSEMLDLTLADVHLTMGFIRCFGKGRKERIVPIGEAAASAIEEYLEKGRGKLLKKQPADALFL |
| | NHHGKKMSRQGFWKNLKKRALEAGIQKELTPHTLRHSFATHLLENGADLRAVQEMLGHADISTTQIYTHV |
| | TKTRLKDVYHKFHPRA |
| 1335 | WP_085361167.1 |
| | MTSVPVLADAVSLPATIAPDLAAAVSYAKAEKAPATRRAYETDFRLFRTYCEEKAASSLPALPETVAAYL |
| | AHGVQEGAKASTLGRRLAAIRYAHKLASLPTPTDSEAVKATLRGIRRTIGAAKVKKAPAVASRIKAMVAA |
| | CPSTIAGKRDRALLLLGFGGAFRRSELVALDVEHIEETSEGLLILIAKSKTDQDAEGVTIAVARGSAETC |
| | PVVALRDWLDAAGIDAGPVFRPINKAGVVSAERLTDQSVALIVKAYARRVGLDAGVFSGHSLRRGFLTSS |
| | AAAGKSIFRMKDVSRHKSVDTLAGYIQEAELFKEHAGAGLL |
| 1336 | WP_007858208.1 |
| | MEKTGREITQELLSGFCIHLEESGYAKATVNKYKADLMQYILFLEGAPVCEEGLSRYREYLEQQYRTSSA |
| | NSKIAAVNAFFKSVGWEYLIPALEPGESLPVMGEELTLSEYRQLLKEAKQQGNLRLYYLIQILSSTKINI |
| | SEHRYVTVEAVSRGYMVIPRGKRSRVIFIPDRLRRQILTYCKKQEIQSGPVFVNWKGTPLDRSNVHKYLK |
| | RLSQNAGVDPEKVNPRSLTRVVEFSSAVYMLDEKMAGEVQDP |
| 1337 | WP_046027227.1 |
| | MDVSNNTNQPISATETRLELTEIASSTQATAEAFIAAGTAANTVRSYRSALAYWEAWLHLRYDRALGDGA |
| | LPAPVVVQFIVDHLARPTPDGTWHHLLPPNIDLALCQTRVKGKPGPLAFNTVSHRLAVLAKWHKLQHWDN |
| | PCAASAVVTLLREAGKAQVRQGVGVRKKTAMTREPLQAMLATCTDGLRGVRDKALLLLAWSGGGRRRSEV |
| | VNLQVGDVRKLDDDTWLYTLGATKTDTGGVRREKPLRGPAAQALSAWLAVAPADCGPLFRRMYKGNKVGV |
| | APLSADQVARIVQRRAKLADLEGDWAAHSLRSGFVTEAGRQGVPLGEVMAMTEHRSVATVMGYFQTGSLL |
| | SSRATELLPKADSKEQGE |
| 1338 | OUV98802.1 |
| | MNKLTTDLKLLHEETLNNLRSSKANNTLRAYKSDFKDFGIFCAKHGLNALPTEPKIVSLYITYLSKNSKI |
| | STLRRRLVSISMVHKLKGHYLDTKNPVIVENLMGIRRVKGSIQKGKKPILIKHLKS1INIINDQKIDEIK |
| | KLRDKSIILIGFGGGFRRTELISIDYEDLEFVPEGLKINIKRSKTDQFGEGMIKGLPLFINEVYCPVSNL |
| | RKWLEVSKIKSGPIFTRFSKGLSLTNKRLTDQSVVLLIKEYLKLAGIENTNFAGHSLRSGFATVAAESGA |
| | DERSIMAMTGHKTTQMVRRYIKEANIFKNNALNKVKI |
| 1339 | WP_075500861.1 |
| | MNELTTELKSLHEATLNNLKSSKANNTLRAYKSDFKDFGIFCAKHGLKTLPSEPKIVSLYLTHLSKNSKI |
| | STLRRRLVSISMVHKLKGHYLDTKHPIIVENLMGIRRVKGSIQKGKKPILISHLKSIINVIDEQKIENIK |
| | KFRDKSLILIGFGGGFRRTELISIDHEDLEFVPEGLKITIRKSKTDQLGEGMIKGLPYFTNETYCPIVNL |
| | KKWLEISKIKSGPIFRRFSKGLSLTDKRLTDQSVVLLMKEYLKLAGIENKNFAGHSLRSGFATVAADSGA |
| | DERSIMAMTGHKSTQMVRRYIREANIFKNNALNKVKI |
| 1340 | WP_011906504.1 |
| | MILMPQDDPALNVIPAGLSPEDDFLPVLAGGELPLSPARAYLLSLNSPRSRQTMASFLNIVAGMLGAASL |
| | ETCSWGSLRRHHVMGVTELLRDTGRATATVNTYLSALKGVAKEAWMLKLMDVESFQHIRAVRNLRGSRLP |
| | RGRALPAEEIGKLFAVCEADATYLGVRDAALLGVILGCGLRRSETVGLSLSDVVTHERALRVLGKGNKER |
| | LAYMPAGTWQRLQTWIDQVRGEAAGPLFTRIRRFDTLTNDRLTDQAVYHILQMRQRQAQIERCAPHDLRR |
| | TFATAMLDNGEDLITVKDAMGHASVTTTQQYDRRGEERLRQARDRLNLT |
| 1341 | WP_014269099.1 |
| | MIASAPFTLCKLSKNDRIYWYALFRDPQTGKRTNKKSVEKLRKELGIQSTQPIKRRDEAILICKQALDAG |
| | LLFGKKTPTTLFDYLSLFFDWEKSPYVEKRNLLDPGSLSQDYISTRQNLVSNHVLPLIHHNLLLSVVTTR |
| | YMEQLQLSLVKKGKLSHATVNICMQAVTMAVREAQRAGLIDASVSIALRPLKCTHRMRGILSEDELSNFM |
| | QYLKTSGEKRMYLACLLSLLTGMRSGELRGLHASSISSGLITVEFAYANKAGLKEPKGKKTRLVPCPAFL |
| | CEELLLLGRSNPFGNGNDLVFWSRRTGSYVSSHYFSEKLQGALVRSKVLEKQEILDRNITFHSLRHMANT |
| | LLRGSVDEHVLRMTIGHSSEQLSDLYTHLSQRGLKSVELAQQNNILPLLGENRE |
| 1342 | WP_002328898.1 |
| | MKNTEIYQKIDTIILHMPDYIKDFVQDREDKDQSPRTTLEYLKNYKLFYEWLLSESIVPDTSITSIRHLT |
| | AYDLNLYKSHLKRRAKENVKKDTTKAKLEDNNNLGLSTSTINRNITALKVLFKYLSKSSNNPLGKPYLED |
| | NPMDQVATITDKTTLAARANAIEKKLFLDEDTQNYLDYIANEYKNTLSKRALIYYHRDVERDLAINALIL |
| | GSGLRLSEVVNINLDDLSLDKNNVVVTRKGNKRDAVNIAAFAMEYLANYLAIRKERYKVTENEKALFLAI |
| | YQGEAKRISGIAIERMVAKYSKGFRVQVSPHKLRHTLATRLYQQTNSLVLTAQQLGHSSTNTTTLYTHID |
| | NAATIDALNSL |
| 1343 | WP_051279402.1 |
| | MTTLTPQPSFSGVPAELQKSFEAAIGYLRAQLAPATLRAYQSDFRIFCDWCERFKLATTPATPETLALFL |
| | SDQADSGVAAVTLERRLASIRYVHQMKELPSPTDHPLVRGTLTGIRRIHGTLPRNQKEPILDHQVFRMLQ |
| | LTPDTLLGLRDRAIIALGFAGAFRRSELAALDVSDLKFDQAGNLVCLIRRGKTDQGGSGFEKPILNGRRL |
| | QPVTHLKAWLSTAGIDEGAVFRRVDWAGAATEQRLSAQWMARVVKNYANQIGLGFTEFGAHSLRSGFITS |
| | AGERDVQLYKIMEVTGQKDPRTVLRYLRRANLFKDHAGEDFL |
| 1344 | WP_058002297.1 |
| | MLDRLKDFIHFMVVEKGLSKNTIVSYERDLKSYLTYMVKVEQIQSLNEITRIHIVHFLHHLKQQGKSAKT |
| | LARHVASIRSFHQFLLREKVTENDPSVHIETPQTERSLPKVLSLTEVEALLDAPSEKGPLPLRDKAMLEL |
| | LYATGIRVSELINLNLDDLHLTMGFVRCIGKGNKERIIPIGKTATVVLEEYIKDGRPKLSSKQRQTEALE |
| | LNHHGNRLTRQGFWKILKGLAKKANIEKELTPHTLRHSFATHLLENGADLRAVQEMLGHADISTTQIYTH |
| | VTKTRLKDVYAKHHPRA |
| 1345 | WP_014080879.1 |
| | MKLPNSYGSVIKLGGKRRKPYAVRISKLVEDDTGKVKRKYTYLAYFSKPEMAYTYLAEYNSGAVVPEHMK |
| | YSDSPTFAEMYEKWKKYRKSLKNQISDSTWRNYEIAFHHFSELHDRKFISIRTNDLQQCLNAYNHKSQTT |
| | ISSMRAVLKAMYQYAKLNEYIDRDLTEGLVYEWTNSTEQIHDRYSDEEIKTLWSKLYEINNVDIILIMIY |
| | TGLRPTELLEIQTENVHLDEKYMVGGMKTEAGKDRIIPLNDKIIPLVKNRYDPNKKYLINNKFGNHYTYG |
| | TYMNGNFNTCMGKLKMKHLPHDGRHTFASLMDSAGANDVCIKLIMGHSMKNDTTKGTYTHKTLEELLTEV |
| | NKI |
| 1346 | WP_034465437.1 |
| | MATEKITKRIVDALKAPKPSRDGVKVREHFVWDRELRGFGVQVMPSGLKSFVIQYRTPEGRNRRAVIGRY |
| | GLMTVEEARKLAHEKLVAVSKGVDPVAEEAKAAGLLTVAEVCDWYLAEAEAGRILGRRRRPIKPSTLAMD |
| | RSRIEAHIKPLLGRRQVASLKLGDVEGAQADIAAGKTSKPRAGSRGGATTGGDGVAARTMSTLHSIFEHA |
| | VRLGKIEANPAKGVRRLASAPRERRLSRSEIERLGKTLRAAAQEGEHPTGLAAIRFLLLTGFRRMEALGL |
| | QRTWLDEEECAIRFPDTKSGAQIRVIGQAAIDLLLDQPKTKSPFFFPADWGEGHFIGVVRVLDRVCQKAG |
| | LADITPHTLRHTYASLAGDLGFSELTIAALLGHSARGVTQRYVHIDEALRMTADQVADEMADLLDGRATP |
| | SRSRSSRRGRSERKLEATGA |
| 1347 | WP_015045988.1 |
| | MAKSGQARVPTAEQQQHLFQVIQEHRHPEKNTAIMQISFKLGLRAQEIALLQIKEVAKLNSLGSGFKLLE |
| | VMSLPAAYTKGADAMNRSKTVYQRRTVSFDVETFNKIIKQVEILAKSGAAVNPEDFYPPLKKHKGKSRDL |
| | PMVDGSLREALTNYIQMRIAKGEVLKPSSPLFITQKGGSYSPNTLQEHMALMLRDWAGIEKASSHSGRRA |
| | LITHIIHKQRKSVKIAQKIAGHVNPSTTLIYEDPPEAVLEDALNDLN |
| 1348 | WP_125440493.1 |
| | MENSSLLPVVVPTRSHSLVELPPSVARYVEAGLHGAENTKRGYAADLRSFQDYCEHHQVLHLPAEVTTVA |
| | GYVSQMADRGMKLATIRRHVAAIAKLHQLAGQPSPTGHEALQVVLDGIARLVGKRQRQAPAFTVAELKQS |
| | IRAMDVTTPTGLRDRALLLLGFAGAFRRSELVALNVEDVELTRQALVIHLRQSKTNQYGLEEDKAVFYSP |
| | SADFCPVRAVQEWIESLGRTSGPLFTRMSRGTQVRPAQPGQHRLTDQSVNDLVQRHLGISYSAHSLRASF |
| | VTIAVEAGQSNKAIKNQTKQKTDAMIERYARLDDVKRFNAAQYLGL |
| 1349 | TDN36797.1 |
| | MENPSSLPVIVPTRSHSLVEMPASVGRYVEAGLQGAANTKRGYAADLRSFEDYCQHHQLSYLPADVSTVA |
| | GYVSQLADRGKKYATIRRHVAAIAKLHQLAGQPSPTSHEALGVVLDGVARVHGKRQRQAPAFTVAELKQA |
| | IRALDLSTPTGLRDRALLLLGFAGAFRRSELVALNVEDVELTRLALVIHLRRSKTNQYGEEEDKAVFYAP |
| | SADYCPVRAVQDWLAVLARPAGPLFTRMSRGTSRRPAQPGTARLSDOSVNDLVQRHLGSSYTAHSLRASF |
| | VTVAVEAGQSNKAIKNQTKQKTDAMIERYARLDDVKRFNAAQYLGL |
| 1350 | WP_133659153.1 |
| | MPASVGRYVEAGLQGAANTKRGYAADLRSFEDYCQHHQLSYLPADVSTVAGYVSQLADRGKKYATIRRHV |
| | AAIAKLHQLAGQPSPTSHEALGVVLDGVARVHGKRQRQAPAFTVAELKQAIRALDLSTPTGLRDRALLLL |
| | GFAGAFRRSELVALNVEDVELTRLALVIHLRRSKTNQYGEEEDKAVFYAPSADYCPVRAVQDWLAVLARP |
| | AGPLFTRMSRGTSRRPAQPGTARLSDQSVNDLVQRHLGSSYTAHSLRASFVTVAVEAGQSNKAIKNQTKQ |
| | KTDAMIERYARLDDVKRFNAAQYLGL |
| 1351 | OUW60929.1 |
| | MKSLVTDLKSLELETLKNLKNSKADNTLRAYESDFKDFAAFCKSNGFSSLPTEPRILALYLTHLSVNSKY |
| | STLKRRLASISVIHRLKGHYIDTKHPLIIENLLGIKRRKGSSQKSKKPILISDLKLIIKAIDQSELKYLK |
| | KLRNKALILTGFSGGFRRSELVAIEHEDIEFVSEGVKIYVKRSKTDQSGEGMIKAIPYFDNEDFCPVTNL |
| | KNWISQGNIQNGKIFNISDKNVVLIIKKFAGLAGLDQNKYAGHSLRSGFATSTAESGAEERSIMSMTGHK |
| | TTOMVRRYIKEANLFKNNALNKIKL |
| 1352 | WP_008916347.1 |
| | METKQINPLIEHFLDTIWLEQDLAENTLASYRIDLQLLDKWLEANELNLENVQSIDLQSFLAERIESGYK |
| | AASSARLLSSIRRLFQYFYREKIRLDDPSAVIAAPKIPQRLPKDLSEQQVEDLLNAPATEDPLELRDKAM |
| | LEVLYACGLRVSELVGLTFSDISLRQGVIRVVGKGDKERLVPLGEEAIYWIEKYIQEGRPDLLKGKASDV |
| | LFPSKRGTKMTRQTFWHRIKHYAVIANIDSESLSPHVLRHAFATHLLNHGADLRVVQMLLGHSDLSTTQI |
| | YTHVATERLRTLHEQHHPRG |
| 1353 | WP_016800355.1 |
| | MRKTVPILTDFVTINSFVEKLNSKATIEIIDELTGHQYSHNSLLGIYSDWNRYHAFCTKHRINTLPASIT |
| | AVRRFLETESNDRKYASLKRYTATLSLLHTVLNFANPIKHRQVRFTLLHLQAQMAGDAKQTNAMTSAHLT |
| | ELNMLLSHQKANLKEVRDIAIYNVMFECALKRSELKALKMNDIESYDEGYQITIKDSAYKLSQVASVALQ |
| | RWLSFTGSEDELPMFRAIDKHENIRLQPLDDSSIYRILRRASDILGLADNHHFSGNSIRVGAAQELSKQG |
| | LKVREIQDFGRWLSPAMPAQYVGYTGTAESEKMKFKAIVPWQ |
| 1354 | WP_029203706.1 |
| | MSIRNLKDGSTKPWLCECYPNGRTGKRVRKKFTTKGEAKAFELHTMKEIDDKPWMGSKTDHRRMSELLDT |
| | WWTIHGHTLKSGKQARELIAKTIEELGNPIASHLKERDYLDYRAARIPYRGKNKSIKISPTTHNTELIYL |
| | KGMFKKLIKYNQWKYPNPLEAIETIKTSEKNLAYLTKPQIEEFLVNLKNFNRVITVSIPQLIVISKICLA |
| | TGARISEALTLTRSQVAEFKLTYTETKGKRNRSVPISPALYQEILDIAVSDHEIFNTSYKDAWRYIKKAL |
| | PEHVPSGQATHVLRHTFASHFMMNKGDILVLQRILGHTKIEQTMAYSHFAPEHLIQAVHLNPLEN |
| 1355 | WP_030064747.1 |
| | MTEIEHYTPAAPPAVRQLSPEAQAALAAGRADSTRRAYAEDRSAYLAWCAERGEQPLPASQDLLVEYVTH |
| | LTLTPRPRTGRPSAPSSLERMLSAITTMHAELDLPKPVTKGARTVIAGYKHKLALDKKPGGKQRQVKPAL |
| | PPALRKMLDALDRDTLIGKRDAAMLLLGYSAATRSSELVGLDIGEPVECDEGYLVSIYRVKMKKFTESAI |
| | PYGKNPATCPVRALRSLIAAMREAGRTEGPLFVRIDRHGRIAPPMVRHGKPIGDPSGRLTADAASDVIER |
| | LAEAAGFMGRWRGHSLRRGFATAAQRGGAPMVRVARQGGWADNSTSLARYFDEGDPWEDNPVTGL |
| 1356 | WP_048474244.1 |
| | MPRSDSQPESPVVAYPGWFTDFLDDRVIRKPSPHTTKAYRQDFEAIATLVAGQAEDVVNLEAAALDKDTL |
| | RAAFAVYARTHSAASIRRCWSTWNTLCTYLFTAELLGANPMPLIGRPKVPKSLPKSYSDNTVTGLVTAID |
| | ADTGSARDSDWPERDRAIVFTALLAGPRAEELIRADIGDVRRTDDGGGVLHVRGKGNKDRRIPFGKELLD |
| | VIEQYLESRVVRFPPARRRVPDSDTLSRFSSNAPLFVGVDGERITRGTLQYRILRAFKRAGINSERPAGA |
| | LVHGLRHTFATELANAHVSVYTLMKLLGHESMVTSQRYVDGAGTETRSATDKNPLYRFLSPRTEYSNQPV |
| | DSRGVQGS |
| 1357 | WP_109314041.1 |
| | MSTHAPYLPAASPALSVEDQEALTDLYVRGTPANTLRAYERDLLYVTAWKTARFDLALRWPESEATALAF |
| | ILDHARDLSDAPSDDHSRQVAEVLIAQGLRKSLACPAPSTLDRRIASWLAFHRMKNLESPFGSPQVNQAR |
| | SKARRAAARPPTPKSAHPITRDILELLLATCRGSRRDCRDRAILILGWASGGRRRSEITGLMFEDVSLKE |
| | FGEKSLVWISLLETKTTAKGKTPPLVLKGRAALALVHWIEVGQIKNGPLFRPVSKADRVLKRRLSPDGIY |
| | QIVKHRLRLAGLPEDFASPHGLRSGFLTQAALDGAPIQTAMRLSLHRSMAQAQKYYDDVDVAENPATDLL |
| | G |
| 1358 | WP_029224390.1 |
| | MSIRNLKDGSTKPWLCECYPNGRTGKRVRKKFTTRGEAKAFELHTMKEIDDKPWMGSKPDHRRMSELLDA |
| | WWTIHGHTLKSGKQARELIAKTIEELGNPIASHLKERDYLDYRAARIPYRGKNKSIKISPTTHNTELIYL |
| | KGMFKKLIKYNQWKYPNPLEAIETIKTSEKNLAYLTKPQIEEFLVNLKNFNRVITVSIPQLIVISKICLA |
| | TGARISEALTLTRSQVAEFKLTYTETKGKRNRSVPISPALYQEILDIAVSDHEIFNTSYKDAWRYIKRAL |
| | PEHVPSGQATHVLRHTFASHFMMNKGDILVLQRILGHTKIEQTMTYSHFAPEHLIQAVHLNPLEN |
| 1359 | WP_010646715.1 |
| | MSTVQAISDKRVVKKAEKYLKRHHDEVYWLIWRIGIETGLRITDITKLSYDNINFESGEVTVIESKGTLA |
| | RQARARHKVLKSVKNELLNYYKRDHAKLLSVYVCDYRNIVDLVPRSWKHSIEVRLEEATKSAPVKKRVAY |
| | LSSRTLTALKKRRKLWLGKDSGLIFSRATLASNRAKRQRGVISRQACWRVFSCLSCCIDELRQHKIGCHS |
| | LRKIFARHLYHSSDMDIGLVATIIGHQSVSTTLRYIGISDEDTKRAQLRLFDYFFA |
| 1360 | WP_021710415.1 |
| | MSIRNLKDGSKKPWLCECYPYGRTGKRVRKRFTTKGEAKAFELHTMKEIDDKPWMGIKPDNRRMSELLET |
| | WWTIHGHTLKSGKQARDLISKTIEELGNPIACQFKERDYLAYRAARIPYRGKNKSIEISPTTHNLELIYL |
| | KGMFKKLIKYNQWKYPNPLEAIEPIKTSEKHLAYLTKPQIEEFFDNLQNCNRVIKASIPQIIVIAKICLA |
| | TGARISEALTLTRTQITELKLTYTDTKGKRNRSVPISPSLYQEILDIAVSDHDIFNTSYKDAWRYIKRAL |
| | PEHVPNGQATHVLRHTFASHFMMNKGDILVLQRILGHTKIEQTMAYSHFAPEHLIQAVHLNPLEN |
| 1361 | WP_011999282.1 |
| | MSVRNLKDGSTKPWICECYPNGRAGKRVRKKFATKGEAKAFELHTMKEIDDKPWMGIKPDNRRMSELLEN |
| | WWTIHGHTLKSGKQAKDLISKTIEELGNPIACQFKERDYLAYRAARTPYRGKNKSIEISPTTHNLELIYL |
| | KGMFKKLIKYNQWKYPNPLEAIEPIKTSEKHLAYLTKPQIDEFFDELQNCKRVIKASIPQIIVIAKICLA |
| | TGARISEALTLTRTQITEFKLTYTDTKGKRNRSVPISPSLYQEILDIAVSDHDIFNTSYKDAWRYIKRAL |
| | PEHVPNGQATHVLRHTFASHFMMNKGDILVLQRILGHTKIEQTMAYSHFAPEHLMQAVHLNPLEN |
| 1362 | WP_050649239.1 |
| | MSVRNLKDGSTKPWICECYPNGRTGKRVRKKFATKGEAKAFELHTMKEIDDKPWMGIKPDHRRMSELLDT |
| | WWNIHGHTLKSGKQARDLIAKTIEELGNPIACQFKERDYLAYRAARIPYRGKNKSIEISPTTHNLELIYL |
| | KGMFKKLIKYNQWKHPNPVESIEPIRTSEKNLAYLTKPQIEEFLFNLKNFNRVITVSIPQLIVISKICLA |
| | TGARISEALTLTRSQVAEFKLTYTETKGKRNRSVPISPALYHEILDIAVNDHKIFDTTYKDAWRYIKRAL |
| | PNHVPSGQATHVLRHTFASHFMMNKGDILVLQRILGHTKIEQTMAYSHFAPEHLMQAVHLNPLEN |
| 1363 | WP_051941091.1 |
| | MIPQDQPLEDTKQGSTLPSAGLEPAAQQAVRELLREGESTNTRNSYQSAMRYWAAWHALRFERQMQLPLD |
| | VPCVLQFIIDHALRQTGAGLASEMPAHMDRALVEAGYKAREGPLSHNTLVHRMAVLSKAHQVHGLANPCQ |
| | DGAVRELMSRTRKAYARRGEQPAKKDALTRDLLEQLLQTCDDSLRGRRDRALLLFAWSSGGRRRSEVAGA |
| | DMRHLRAVGPQEFIYTLAHSKTNQSGRDAPENHKPVTGRAAQALADWLRAAAIQEGPIFRRIRKGGHVGE |
| | PLSPAAVRDIVKQRCALAGVEGDFSAHSLRSGFVTEAGRQNVPLPDTMALTGHSSVNTVLGYFRADSALS |
| | NRAARLLDAGDDDAAAAAQGSGRPQS |
| 1364 | WP_065347010.1 |
| | MGTITTRKRADGSQSYTAQIRLKEGGQIIYSEAQTFSRKVLASEWLRRREYELEQERASGQALHKKVSVG |
| | ELLRDYVSAAENVTEWGRSKKADIARVQASGLADLQATKLTVQDLMGYAKKRRTEDEAGPATVLNDMVWL |
| | RQVFLHASAARGIDAPLQVLDRAKSELLRTRVIAKPAQRSRRLLPEEEAKLLEHFSSRDGRASIPMSDIM |
| | QFALLTARRQEEICRLRWVDVDFEKGVAWLDDVKHPRMKKGNRRCFRVLNAAADIIKSQSREEGVEFVFP |
| | YNNRSVGAAFTRACHVLGIEDLHFHDLRHEATSRLFEKGYSIQEVAQFTLHESWATLKRYTHLRPENVQE |
| | R |
| 1365 | WP_049681475.1 |
| | MNDLTNFNHLTSEQYLTQLQNKLEHRHLLDEHRNLSLSDSSEQDFLELFFSEKVFTPDKEFSPHTIRAYR |
| | SDAKTLLQFLMEHSLSFRNIGFPEVKVYNKYIKEKYAPKSAIRKLEFFRRLLDFGYETQFYKAHLSTWIS |
| | KPTSKKGHYIIEETRLEAEQTRVQVRELNQKDAEYLISCFPKIVKANTNREQLEKRNLLIGYLLYTTGLR |
| | ASELVSLNWGSFRYNRQGHLYADVIGKGKKPRSIPVKDETIELLFDYRKSLGESVEINPEDVNPLFFALY |
| | NKKEPCEHKKRLTYPSLYKIVKEAVHLAGKNSKVSPHWFRHTFVTMLLENDVPLAVVKDWAGHSDISTTN |
| | IYLERVNQDNTHVYLNKVNVFK |
| 1366 | WP_025315261.1 |
| | MAVLTDYKINASKSKAKEYTLKDGNGLFLNIHPNGSKYWLFRFSWNGKQTRMSFGTYPTVDIKQARYLCE |
| | QANFKLLSGIDPRLKENPTIDPVDEVLDEEPKCTFAQFAQHWLEFKMKKLNAKPSKDKKNNGRGSTEIQI |
| | RRAFTNDIFPVLKDKSIHKVTRNDLLCIIRKVEKRGALSVAEKIRSWLDEIFRYAVVTEGLEINPAADLD |
| | IASLPYRRNNRYPFIDVSELPELLVKLSTYQGSRLTILGLRLLLLTGVRTGELRFSEAWQFDLKNALWRI |
| | PASDVKQLQQVIEKVDNRVPDYIVPLSRQALDIVKELLSYHMRGQRYLIANRTNPLEAMSENTLNQALKN |
| | MGFKRRLCTHGIRHTISTALNDLKYDKDFIEAQLSHSDTNKVRATYNHAQYIEPRREMMQEWADLLDKWE |
| | QEVLDKINNK |
| 1367 | WP_038069793.1 |
| | MSENNEKSSSSAPNGSSVNEDNERDHRDGDALSLPSFVAGSGTLDRLVDTARDYARAAASDKTLKAYAKD |
| | WAHFARWCRMKGAEPLPPSPEMIGLYLADLASGSGLSPALSVSTIERRLSGLGWNYAQRGFTLDRKNRHI |
| | ATVLAGIKRKHARPPVQKEAILAEDILAMVATLAFDLRGLRDRAILLLGYAGGLRRSEIVSLDVHKDDTP |
| | DSGGWIEIFDKGALLTLNAKTGWREVEIGRGSKDQTCPVHALEQWLHFAKIDFGPIFVGTSRDGKRALET |
| | RLNDKHVARLIKRTVLDAGIRSDLPEKDRLALFSGHSLRAGLASSAEVDERYVQKQLGHASAEMTRRYQR |
| | RRDRFRVNLTKAAGL |
| 1368 | WP_006861039.1 |
| | MDKNQLTYHEQVKVDNTLRMREILKTMPGFARDYFRAIEPTTSTRTRISYAYDIRVFFQFLLEENPSLRG |
| | KEMTDITLDILDKIKPVDIEEYLEYLKVYQSEDGLKTNGERALKRKMVALRGFYAYYFKREMIKTNPTLL |
| | VDMPKIHDKAIVRLDTDETASLLDYIEHAGDSLSGQKKVYWEKTKRRDLALVTLLLGTGIRVSECVGLDI |
| | GDVDFKNNGIKVVRKGGNEMVVYFGDEVEKALRDYLEERCGITPVAGSENALFLSTQRKRIGVQAVENLV |
| | KKYARQITTTKKITPHKLRSTYGTSLYQETNDIYLVADVLGHKDVNTTKKHYAAMDDQRRRSAASAVHLR |
| | EP |
| 1369 | WP_102369017.1 |
| | MSELDRYLNAATRDNTRRSYRAAIEHFEVNWGGFLPATSDSVARYLVAHAGVLSVNTLKLRLSALAQWHT |
| | SQGFPDPTKAPVVRKVLKGIRALHPAQEKQAEPLQLQHLEQVIQFLEQEGHDARGAEDHPRWLRAKRDAA |
| | LILLGFWRGFRSDELCRLNIEHVQAVPGSGITLYLPRSKSDRENIGRTYQTPALLRLCPVQAYSEWLSAS |
| | ALVRGPVFRGIDRWGNLGEEGLHANSVIPLLRQALERAGIAADQYTSHSLRRGFATWAHRSGWDLKSLMT |
| | YVGWKDMKSAMRYVEATPFLGMTRASLE |
| 1370 | WP_003212574.1 |
| | MSELDRYLNAATRDNTRRSYRAAIEHFEVNWGGFLPATSDSVARYLVAHAGVLSVNTLKLRLSALAQWHT |
| | SQGFPDPTKAPVVRKVLKGIRALHPAQEKQAEPLQLQHLEQVIQFLEQEGHDARGAEDHPRWLRAKRDAA |
| | LILLGFWRGFRSDELCRLNIEHVQAVPDSGITLYLPRSKSDRENIGRTYQTPALLRLCPVQAYSEWLSAS |
| | ALVRGPVFRGIDRWGNLGEEGLHANSVIPLLRQALERAGIAADQYTSHSLRRGFATWAHRSGWDLKSLMT |
| | YVGWKDMKSAMRYVEATPFLGMTRASLE |
| 1371 | WP_102604909.1 |
| | MSELDRYLNAATRDNTRRSYRAAIEHFEANWGGFLPATSDSVARYLVAHAGVLSVNTLKLRLSALAQWHT |
| | SQGFPDPTKAPVVRKVLKGIRALHPAQEKQAEPLQLQHLEQVIQFLEQEGHDARRAEDHPRWLRAKRDAA |
| | LILLGFWRGFRSDELCRLNIEHVQAVPGSGITLYLPRSKSDRENIGRTYQTPALLRLCPVQAYSEWLSAS |
| | ALVRGPVFRGIDRWGNLGEEGLHANSVIPLLRQALERAGIAADQYTSHSLRRGFATWAHRSGWDLKSLMT |
| | YVGWKDMKSAMRYVEATPFLGMTRASLE |
| 1372 | WP_008432517.1 |
| | MSELDRYLNAATRDNTRRSYRAAIEHFEVNWGGFLPATSDSVARYLVAHAGVLSVNTLKLRLSALAQWHT |
| | SQGFPDPTKAPVVRKVLKGIRALHPAQEKQAEPLQLQHLEQVIQFLEQEGHDARRAEDHPRWLRAKRDAA |
| | LILLGFWRGFRSDELCRLNIEHVQAVPGSGITLYLPRSKSDRENIGRTYQTPALLRLCPVQAYSEWLSAS |
| | ALVRGPVFRGIDRWGNLGEEGLHANSVIPLLRQALERAGIAADQYTSHSLRRGFATWAHRSGWDLKSLMT |
| | YVGWKDMKSAMRYVEATPFLGMTRASLE |
| 1373 | WP_002892342.1 |
| | MKQETLMKNINGLLEIMPWYVKEYYQAKLVIPYSYKTLYEYLKEYRRFFEWLIRDHEKLGKTARYADYDT |
| | IADVHIDELAHLPKSIIEAYFVYLRENTERRSISEVSIVRTKDALSSLFKYLTQETEDDEGEPYFYRNVM |
| | VKVKIKKPKDTLASRADNMKEKLFLNDTQSFLDYIDNEHEKKISKRAQVSFVKNKERDLAVIALLLSTGV |
| | RLSELVNLDMQDVNLATRTITVIRKGGKKDVVNIAPFGIPYIERYLEIRKGRYAASDSDKAFFLTTQNKV |
| | PARLGTRSVELLVKKLSTAYGKPTTPHKLRHTLATRLYEQTKDSLLVSQQLGHKGTAMVEVYAHVAAETT |
| | KEALSDL |
| 1374 | WP_002887164.1 |
| | MSKQKDKYLALKRQLPDIIDEYISYLQVDVEEPSPKMVERLSVIQKFLNSYAITIDKEGASLSLTDLEKL |
| | PREFVQNYLANLRLKPAGKRFILYTLAAFWNYLTNTSFTIERGMPLFYRNVFNEWKIVYKESYHNIIYSE |
| | SKKKTILYTQEELEGLLDFMANSYVTTLPTQKKADNWEKEKERNIAIFAIIIGTGASTQEVVNLTVRDID |
| | MRKKGIWVVRNNEKQFIRFLPFTIPYIAPFVKERRGRWDLDPSIPPLFLTMLKKPMGRNTIGHLAKNIGH |
| | AYGKVITPSILKDSHASIVYKETGDIKKVAEIQGYSLDKNHLIRFID |
| 1375 | WP_070578346.1 |
| | MSKQKDKYLALRRQLPDIIDEYISYLQVDVEESSPKMVERLSVIQKFLNSYAITIDKEGASLSLTDLEKL |
| | PREFVQNYLANLRLKPAGKRFILYTLAAFWSFLTNTSFTVERGMPLFYRNVFDEWKIVYKESYHNIIYSE |
| | SKKNTILYTQQELESLLDFMANSYVTTLPTQKKADNWEKEKERNIAIFAIIIGTGASTQEVVNLTVRDID |
| | MRKKGIWVVRNNEKQFIRFLPFTIPYIAPFVKERRGRWDLDPSIPSLFLTMLKKPMGRNTIGHLAKNIGH |
| | AYGKAIAPSILKDSHASIVYKETGDIKKVAEIQGYSLDKNHLIRFID |
| 1376 | WP_011530252.1 |
| | MSVQPGTALQLASKWSRPENRRREGLRAAHTQDADTLIDLLNTYIRLKSSRKGRTSALTLKAYAESVRQF |
| | LAFTGPPESPSRALNQLSAEDFEVWLLHLQEAGLKPNTIKRHLYGVRNLMKALVWANVLKADPSAGVSPP |
| | TDPTPAHAKKRALTQAQMRALLALPGELHPEDSVQASRDALLLALGGTLGLRAAEIVGLDLADVDLATGT |
| | LTVRGKGGKTRVVPLPAGVKALLQRWLPARQTVNPKVPALLVSLSSLNRGGRLSTDGARFIAHAYYRQLG |
| | LPPEMWGLHTLRRTAGTHLYRATRDLHVVADLLGHASVTTSAIYAKMDADVRREAVEALERLQQEGSAAV |
| | QPSRIEQQEDAQQQGGQVA |
| 1377 | WP_005834081.1 |
| | MNQEDVRVSFYLKKSEADEQGECPIMGRLNVGKYSEAAFSMKMTAPESAWLSGRATGKSARSREINRQLD |
| | EIRASALSIYQDLFALREKVSAEEVKCILLGMAYGQETLVAFFLSFIKKFEKKVGINREESTATSYKYAC |
| | GQLMQFLNKEYNLSDIPFTALDRSFIDKYDLYLRTDCQLSAGTILLLTTQLMTVIRKAKSAGILTSNPFA |
| | GYEAERPAREIKYLTEHELERIMSTPLHNRKLYHIRDLFLFSCFTGIPYGDMCRLSDEDLVAVEDGTLWI |
| | KTSRKKTKISYEVPLLDIPLYILEKYRDAAPEGKLLPMYSNSELNNALKTIADLCGIKQRLVFHQARHTS |
| | ATTVLLSNGVPLETVSKILGHERISTTQIYAHVTDDKVENDTRMLDAKIAERFSVAI |
| 1378 | WP_100294115.1 |
| | MTYPDISGSAYSSLQTVFDAQLNSRARRFLRSAKADSTLNAYQADTRIFVFWCQLHGVEPLQASHHHIMN |
| | FLADQADGVLADWVWLDKAEGKGELRHGEPRKPATLVRRLAGIRYAFKQKGIHPMPTEHPDIKEMMRGIV |
| | RLGDNHKRKTGALTLEPLAKVLDGIDTHDLAGLRDHTLLLLMFSGALRRSEAARIEVSDLDFVGQGIRLR |
| | LKPSKHQLHETEIALIPGKHYCPVLALNNWLKKSRINSGPLFRRMNRWGQITPDPLGPQGINLMIKRRTG |
| | HSIDYLYVSGHSLRRGFITSAVTAGKPMNKIIEVTRHKDMRTLQEYFDDAHKFADHALDGLL |
| 1379 | WP_041234271.1 |
| | MAYPSLSNPAHQSLQTVFDAQLNSRARRFLRSAKADSTLNAYQADTRIFVFWCQLHGLDPLQTTHHDIMN |
| | FLADQADGVLADWIWLDKEEGKGELRNGEPRKPATLVRRLAGIRYAFKQKGIHPMPTEHPEIKEMMRGIV |
| | RLGDNRKHKTGALTLQPLTQVLDGIDTGDLAGLRDHTLLLLMFSGALRRSEAARIEVSDLDFVGQGIRLR |
| | LKPSKHQLHETEIALIPGKQYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1380 | WP_041202099.1 |
| | MAYPSLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFMGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1381 | WP_088868973.1 |
| | MAYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDSGDLAGLRDHTLLLLMFSGALRRSEAARIEVSDLDFVRQGIRLR |
| | LKPSKHQLHETEIALIPGKHYCPVSALKKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1382 | WP_069554870.1 |
| | MAYPSLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTRDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1383 | WP_103252006.1 |
| | MAYPSLSNPAHQSLQTVFDAQLNSRARRFLRSAKADSTLNAYQADTRIFVFWCQLHGLDPLQTTHHNIMN |
| | FLADQADGVLADWVWLDKEEGKGELRNGEPRKPATLVRRLAGIRYAFKQKGIHPMPTEHPEIKEMMRGIV |
| | RLGDNRKHKTGALTLQPLTQVLDGIDTGDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1384 | WP_127005624.1 |
| | MAYPTLSNPAHQSLQTVFDAQLNSRARRFLRSAKAVSTLNAYQADTRIFVFWCQLHGLDPLQTTHHDIMN |
| | FLADQADGVLAAWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGLRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFADHALDGLL |
| 1385 | SIQ01063.1 |
| | MAYPTLSNPAYQSLQTVFDAQLNSRARRFLRSAKAVSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGLRLR |
| | LKPSKHQLHETEIALIPGKHYCPVSALQNWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1386 | WP_100645880.1 |
| | MAYPTLSNPAHQSLQTVFDAQLNSRARRFLLSAKADSTLNAYQADTRIFVFWCQLHGLDPLQTTHHDIMN |
| | FLADQADGVLADWIWLDKEEGKGELRNGEPRKPATLVRRIAGIRYAFKQKGIHPMPTEHPEIKEMMRGIV |
| | RLGDNRKRKTGALTLQPLTQVLDGIDTRDLAGLRDHTLLLLMFSGALRRSEAARIEVSDLDFMGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1387 | WP_100653772.1 |
| | MTYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPLPTEHPEIKEMMRGIV |
| | RLGDNRKRKTGALTLQPLTQVLDGIDTSDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1388 | WP_041915408.1 |
| | MAYPSLSNPAHQSLQTVFDAQLNSRARRFLRSAKADSTLNAYQADTRIFVFWCQLHGLDPLQTTHHDIMN |
| | FLADQADGVLANWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALIPGKQYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1389 | WP_129504075.1 |
| | MAYPTLSNPAHQSLQTVFDAQLNSRARRFLRSAKADSTLNAYQADTRIFVFWCQLHGLDPLQTTHHDIMN |
| | FLADQADGVLADWVWLDKEEGNGELRNGEPRKPATLVRRLAGIRYAFRQKGIHPMPTEHPEIKEMMRGIV |
| | RLGDNRKRKTGALTLQPLTQVLDGIDTGDLAGLRDHTLLLLMFSGALRRSEAARIEVSDLDFVGQGLRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEITRHKDMRTLQEYFDDAHKFSDHALDGLL |
| 1390 | WP_094698459.1 |
| | MNYPRISNPVQQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVNDLDFVGQGIRLR |
| | LKPSKHQLHETEIALVPGKQYCPVSALARWLKQSRINEGALFRRMNRWGQLTQEPLGPQGINLMIKRRTG |
| | QAIDDLQVSGHSLRRGFITSAVTAGKPMKKIIEVTRHKDIRTLQEYFDDAHKFSDHALDGLL |
| 1391 | WP_106886783.1 |
| | MAYPTISPPAHQSLQTVFDPALNSRARRFLRSAKADSTLNAYQADTRIFVFWCQLHGLDPLQTSHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLTRVLDEIDTTNLAGLRDHTLLLLMFSGALRRSEAARIEVSDLDFVGQGIRLR |
| | LKPSKHQLHETEIALIPGKQYCPVTALSRWLKASRISQGPLFRRMTRWGQLTAEPLGPQGINLMIKRRTG |
| | QVIDDLYVSGHSLRRGFITSAVTAGKPMNKIIEVTRHKDMRTLQEYFDDAHKFSDHALAGLL |
| 1392 | WP_017785358.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1393 | WP_100858303.1 |
| | MTYPSISNPVHQSLQTVFDPQLNSRARRFLRSAKADSTLNAYEADTRIFVYWCQLQQLDPLQTTHHDIMN |
| | FLADQADGILADWVWLDKREGKGELRNGEPRKPATLVRRLAGIRYAFKQKGIHPMPTEHAEIKEMMRGIV |
| | RLGDNRKRKTGALTLQPLTQVLDEIDTSNLAGLRDHTLLLLMFSGALRRSEAARIEVSDLDFVGQGIRLR |
| | LKPSKHQLHETEIALIPGRHHCPVSALRRWLQKSRINEGPLFRRMNRWGQLMPDPLGPQGINLMIKRRTG |
| | QVIDSLYVSGHSLRRGFITSAVTAGKPMNKIIEVTRHKDMRTLQEYFDDAHKFSDHALDGLL |
| 1394 | WP_123246139.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLKPLACVLDEIDASNLAGLRDYTLLLLMFSGALRRSEAARIEVDDLDFVGQGIRLR |
| | LKPSKHQLHETEIALVPGKQYCPVSALARWLKQSRIHEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1395 | WP_043162717.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLARVLDEIDTGNLAGLRDYTLLLLMFSGALRRSEAARIEVDDLDFVGLGIRLR |
| | LKPSKHQLHETEIALVPGKQYCPVSALARWLKQSRISEGALFRRINRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1396 | WP_124249452.1 |
| | MNYPRISNPVOQPLQSVFDPQLNSRARRFLRSAKADSTLNAYQADTRIFVFWCQLHELDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1397 | WP_096119502.1 |
| | MAYPTVLPPVYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLGRVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSDPLGPQGINLMIKRRTG |
| | QAIDDLYVSGHSLRRGFITSAVTAGKPMNKIIEVTRHKDMRTLQEYFDDAHKFSDHALDGLL |
| 1398 | WP_084202652.1 |
| | MTHSTFPSPAQHSLQAVFDSQLNSRARRFLRSAKAGSTLNAYQADTRIFVFWCQLHGLDPLQSTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLTPLACVLDEIDTNSLAGLRDYTLLLLMFSGALRRSEAARIEVDDLQFVGQGIRLR |
| | LKPSKHQLHESEIALIPGQHYCPVSALQCWLKKSRIEAGPLFRRMNRWGQLTADPLGPQGINLMIKRRTG |
| | QAIDDLHVSGHSLRRGFITSAVTAGKPMNKIIEVTRHKDMRTLQEYFDDAHKFSDHALAGLL |
| 1399 | WP_039215813.1 |
| | MNYPHIQVQTQQALQSVFDPQLNSRARRFLRSAKADSTLNAYQADTRIFVCWCQLHELDPLQTTHHDIMN |
| | FLADQADGILADWVWLDKEEGKGELRNGEPRKPATLVRRLAGIRYAFKQKGIHPMPTEHSEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSSISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1400 | WP_124251491.1 |
| | MNYPHIQAQTQQALQSVFDPQLNSRARRFLRSAKADSTLNAYQADTRIFVFWCQLHGLDPLQTTHHDIMN |
| | FLADQADGILADWIWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1401 | WP_025201727.1 |
| | MNYPHIQAQTQQALQSVFDPQLNSRARRFLRSAKADSTLNAYQADTRIFVFWCQLHGLDPLQTTHHHIMN |
| | FLADQADGILADWIWLDKEEGKGELRNGEPRKPATLVRRLAGIRYAFKQKGIHPMPTEHAEIKEMMRGIV |
| | RLGDNRKRKTGALTLKPLACVLDVIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1402 | WP_125729907.1 |
| | MAYPTVSPPVYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDDAHKFSDHALDGLL |
| 1403 | WP_043122983.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLVLMFSGALRRSEAARIEVDDLNFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1404 | WP_073350284.1 |
| | MNYPHIQSQTQQALQSVFDPQLNSRARRFLRSAKADSTLNAYQADTRIFVFWCQLHGLDPLQTTHHHIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMPEPLGPQGINLMIKRRTG |
| | QAIDDLQVSGHSLRRGFITSAVTAGKPMNKIIEVTRHKDIRTLQEYFDDAHKFSDHALDGLL |
| 1405 | WP_103470761.1 |
| | MAYPTLSPSAHQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSSLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSDPLGPQGINLMIKRRTG |
| | QAIDDLYVSGHSLRRGFITSAVTAGKPMNKIIEVTRHKDMRTLQEYFDDAHKFSDHALDGLL |
| 1406 | WP_043134801.1 |
| | MAYPTLAPSAHQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDDAHKFSDHALDGLL |
| 1407 | WP_125606695.1 |
| | MAYPTVSPPVCQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLHVSGHSLRRGFITSAVTAGKPMNKIIEVTRHKDMRTLQEYFDDAHKFSDHALDGLL |
| 1408 | WP_098984054.1 |
| | MAYPTLAPSAHQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSSLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAINDLYVSGHSLRRGFITSAVTAGKPMNKIIEVTRHKDMRTLQEYFDDAHKFSDHALDGLL |
| 1409 | WP_101149134.1 |
| | MNYPHIQAQTQQALQSVFDPQLNSRARRFLRSAKADSTLNAYQADTRIFVCWCQLHGLDPLQTTHHHIMN |
| | FLADQADGILADWVWLDKEEGRGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIAMVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1410 | WP_087755718.1 |
| | MAYPTVSPPIYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSDPLGPQGINLMIKRRTG |
| | QVIDDLHVSGHSLRRGFITSAVTAGKPMNKIIEVTRHKDMRTLQEYFDDAHKFSDHALDGLL |
| 1411 | WP_080891334.1 |
| | MNYPHIQAQTQQALQSVFDPQLNSRARRFLRGAKADSTLNAYQADTRIFVFWCQLHGLDPLLTTHHHIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSTLARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1412 | WP_111587863.1 |
| | MAYPTVSPPVYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKSIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSDPLGPQGINLMIKRRTG |
| | QAIDDLYVSGHSLRRGFITSAVTAGKPMNKIIEVTRHKDMRTLQEYFDDAHKFSDHALDGLL |
| 1413 | ABO90113.1 |
| | MAYPTVSPPVYQSLQTVFDPQLNSRARRFLRSAKAVSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLGRVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | HRRSLCQWPQPATGIHHLGRHRRQAHEQDH |
| 1414 | WP_103243121.1 |
| | MNYPRLQNPVQQSLQSVFDPQLNSRARRFLRSAKADSTLNAYQADTRIFVSWCQLHGLEPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGTRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSEHALDGLL |
| 1415 | WP_124243812.1 |
| | MNYPRLQNPVQQSLQSVFDPQLNSRARRFLRSAKADSTLNAYQADTRIFVSWCQLHGLDPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDAIDTSNLAGLRDYTLLLLMFSGALRRSEAARIEVDDVQFVGQGIRLR |
| | LKPSKHQLHESEIALIPGTRYCPVSALQQWLKRSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1416 | WP_042878486.1 |
| | MNYPRLQNPVQQSLQSVFDPQLNSRARRFLRSAKADSTLNAYQADTRIFVSWCQLHGLDPLQTTHHDIMN |
| | FLADQADGILANWVWLDKEEGKGELRNGEPRKPATLVRRLAGIRYAFKQKGIHPMPTEHAEIKEMMRGIV |
| | RLGNNRKRKTGALTLKPLACVLDEIDTSNLAGLRDYSLLLLMFSGALRRSEAARIEVNDVQFVGQGIRLR |
| | LKPSKHQLHESEIALIPGTRYCPVSALQQWLKKSRIAEGPLFRRMNRWGQLMADPLGPQGINLMIKRRTG |
| | QAIDDLHVSGHSLRRGFITFAVTAGKPMNKIIEVTRHKDMRTLQEYFDDAHKFSDHALDGLL |
| 1417 | WP_005347025.1 |
| | MGRPRKSDTWLPPRVYRGKSAFEFHPRSGGAIRLAPLAATQSAVWAAYEHMMAEQDGDTIKRLVHEFFES |
| | ADFNDLSATTQKDYRKYSIPVIKVFGGMDPARVESPHIRKYMDKRGQNSKVQANREKAFFSRVFRWAYER |
| | GKVKSNPCQGVRQFKEKARTRYITDLEFQAVMDAARPAVRVAMELSYLCAARKGDVLAMRWSQVGEEGIT |
| | IQQSKTSKIQIKAWSPRLIAAIEQAKQLAGSVVRSSYVICKPNGTPYTDNGFNAAWREAVLTAREQTGWP |
| | MDFTFHDIKAKAISDVEGSSRDKQRISGHKTEAQVAAYDRSIEVVPAVDSVKKR |
| 1418 | WP_042062922.1 |
| | MQTVFDAQLNSRARRFLRSAKADSTLNAYQADTRIFVFWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTHDLSGLRDHTLLLLMFSGALRRSEAARIEVSDLDFVGQGIRLRLKPSKHQLHETEI |
| | ALIPGKHYCPVSALQNWLRKSRISEGPLFRRMNRWGQLMTEPLGPQGINLMIKRRTGQAIDDLYVSGHSL |
| | RRGFITSAVTAGKPMNKIIEVTRHKDMRTLQEYFDDAHKFSDHALDGLL |
| 1419 | WP_042055087.1 |
| | MQTVFDAQLNSRARRFLRSAKADSTLNAYQADTRIFVFWCQLHGLDPLQTTHHDIMNFLADQADGVLADW |
| | VWLDKEEGKGKLRNGEPRKPATLVRRLAGIRYAFKQKGIHPMPTEHPEIKEMMRGIVRLGDNRKRKTGAL |
| | TLQPLTQVLDGIDTGDLAGLRDHTLLLLMFSGALRRSEAARIEVSDLDFVGQGIRLRLKPSKHQLHETEI |
| | ALIPGKQYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1420 | WP_075113648.1 |
| | MQTVFDAQLNSRARRFLRSAKADSTLNAYQADTRIFVFWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTHDLAGLRDHTLLLLMFSGALRRSEAARIEVSDLDFVGQGIRLRLKPSKHQLHETEI |
| | ALIPGKHYCPVSALQNWLHKSRISEGPLFRRMNRWGQLMAEPLGPQGINLMIKRRTGQAIDNLYVSGHSL |
| | RRGFITSAVTAGKPMNKIIEVTRHKDMRTLQEYFDDAHKFSEHALDGLL |
| 1421 | WP_069526884.1 |
| | MQTVFDAQLNSRARRFLRSAKADSTLNAYQADTRIFVFWCQLHGLDPLQTTHHDIMNFLADQADGVLADW |
| | IWLDKEEGKGELRNGEPRKPATLVRRLAGIRYAFKQKGIHPMPTEHPEIKEMMRGIVRLGDNRKRKTGAL |
| | TLQPLTQVLDGIDTHDLAGLRDHTLLLLMFSGALRRSEAARIEVSDLDFVGQGIRLRLKPSKHQLHETEI |
| | ALIPGKHYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1422 | WP_050547838.1 |
| | MQTVFDAQLNSRARRFLRSAKADSTLNAYQADTRIFVFWCQLHGLDPLQTTHHDIMNFLADQADGVLADW |
| | IWLDKEGGKGELRNGEPRKPATLVRRLAGIRYAFKQKGIHPMPTEHPEIKEMMRGIVRLGDNRKRKTGAL |
| | TLQPLIQVLDGIDTNDLAGLRDHTLILLMFSGALRRSEAARIEVSDLDFMGQGIRLRLKPSKHQLHETEI |
| | ALIPGKHYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1423 | WP_076491768.1 |
| | MQTVFDAQLNSRARRFLRSAKAVSTLNAYQADTRIFVFWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTGDLAGLRDHTLLLLMFSGALRRSEAARIEVSDLDFVGQGLRLRLKPSKHQLHETEI |
| | ALIPGKHYCPVSALQNWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1424 | SQH59660.1 |
| | MQSVFDPQLNSRARRFLRSAKADSTLNAYQADTRIFVSWCQLHGLDPLQTTHHDIMNFLADQADGILANW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTSNLAGLRDYTLLLLMFSGALRRSEAARIEVNDVQFVGQGIRLRLKPSKHQLHESEI |
| | ALIPGTRYCPVSALQQWLKKSRIAEGPLFRRMNRWGQLMADPLGPQGINLMIKRRTGQAIDDLHVSGHSL |
| | RRGFITSAVTAGKPMNKIIEVTRHKDMRTLQEYFDDAHKFSDHALDGLL |
| 1425 | WP_071910168.1 |
| | MQTVFDAQLNSRARRFLRSAKANSTLNAYQADTRIFVFWCQLHGLDPLQTTHHDIMNFLADQADGVLADW |
| | VWLNKEEGRGELRNGEPRKPATLVRRLAGIRYAFKQKGIHPMPTEHPEIKEMMRGIVRLGDNRKRKTGAL |
| | TLQPLTQVLDGIDTNDLAGLRDHTLLLLMFSGALRRSEAARIEVSDLDFVGQGIRLRLKPSKHQLHETEI |
| | ALIPGKHYCPVAAIGNWLKKSRINEGPLFRRMNRWGQLTPDPLGPQGINLMIKRRTGQAIDDLYVSGHSL |
| | RRGFITSAVTAGKPMNKIIEVTRHKDMRTLQEYFDDAHKFSDHALDGLL |
| 1426 | 0FC44115.1 |
| | MQSVFDPQLNSRARRFLRSAKADSTLNAYQADTRIFVCWCQLHELDPLQTTHHDIMNFLADQADGILADW |
| | VWLDKEEGKGELRNGEPRKPATLVRRLAGIRYAFKQKGIHPMPTEHSEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | ALVPGKQYCPVSALARWLKQSSISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1427 | AHV35191.2 |
| | MQSVFDPQLNSRARRFLRSAKADSTLNAYQADTRIFVFWCQLHGLDPLQTTHHHIMNFLADQADGILADW |
| | IWLDKEEGKGELRNGEPRKPATLVRRLAGIRYAFKQKGIHPMPTEHAEIKEMMRGIVRLGDNRKRKTGAL |
| | TLKPLACVLDVIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1428 | EKB28734.1 |
| | MQSVFDPQLNSRARRFLRSAKADSTLNAYQADTRIFVFWCQLHGLDPLQTTHHDIMNFLADQADGILADW |
| | VWLDKEEGKGELRNGDPRKPATLVRRLAGIRYAFKQKGIHPMPTEHAEIKEMMRGIVRLGDNRKRKTGAL |
| | TLQPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1429 | OCA67852.1 |
| | MQSVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTGNLAGLRDYTLLVLMFSGALRRSEAARIEVDDLNFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1430 | KMK90327.1 |
| | MQSVFDPQLNSRARRFLRSAKADSTLNAYQADTRIFVFWCQLHGLDPLQTTHHDIMNFLADQADGILADW |
| | VWLDKEEGKGELRNGDPRKPATLVRRLAGIRYAFKQKGIHPMPTEHAEIKEMMRGIVRLGDNRKRKTGAL |
| | TQQPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1431 | APJ17493.1 |
| | MQSVFDPQLNSRARRFLRSAKADSTLNAYQADTRIFVFWCQLHGLDPLQTTHHHIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMPEPLGPQGINLMIKRRTGQAIDDLQVSGHSL |
| | RRGFITSAVTAGKPMNKIIEVTRHKDIRTLQEYFDDAHKFSDHALDGLL |
| 1432 | WP_059167796.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLAKVLDEIDTSNLAGLRDHTLLLLMFSGALRRSEAARIEVSDLDFVGQGIRLRLKPSKHQLHETEI |
| | ALIPGKQYCPVSALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1433 | PKD25755.1 |
| | MQSVFDPQLNSRARRFLRSAKADSTLNAYQADTRIFVCWCQLHGLDPLQTTHHHIMNFLADQADGILADW |
| | VWLDKEEGRGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | AMVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1434 | WP_052101192.1 |
| | MQAVFDPQLNSRARRFLRSAKADSTLNAYEADTRIFVYWCQLQQLDPLQTTHHDIMNFLADQADGILADW |
| | VWLDKQEGKGELRNGEPRKPATLVRRLAGIRYAFKQKGIHPMPTEHAEIKEMMRGIVRLGDNRKRKTGAL |
| | TLQPLIRVLDDIDTSTLAGLRDHTLLLLMFSGALRRSEAARIEVSDLDFVGQGIRLRLKPSKHQLHETEI |
| | ALIPGKQYCPVSALANWLKKSRIGEGPLFRRMNRWGQLMPEPLGPQGINLMIKRRTGQVIDDLYVSGHSL |
| | RRGFITSAVTAGKPMNKIIEVTRHKDMRTLQEYFDDAHKFSDHALDGLL |
| 1435 | WP_052159026.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLARVLDEIDTSNLAGLRDHTLLLLMFSGALRRSEAARIEVSDLDFVGQGIRLRLKPSKHQLHETEI |
| | ALIPGKQYCPVSALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1436 | AGM44110.1 |
| | MQSVFDPQLNSRARRFLRSAKADSTLNAYQADTRIFVFWCQLHGLDPLQTTHHHIMNFLADQADGILADW |
| | VWLDKEEGRGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEITRHKDIRTLQEYFDDAHKFSDHALDGLL |
| 1437 | WP_042654758.1 |
| | MQAVFDPALNNRARRFLRSAKADSTLNAYQADTRIFVFWCQLHGLDPLQTSHHDIMNFLADQADGILADW |
| | VWLDREEGKGELRNGEPRKPATLVRRLAGIRYAFKQKGIHPMPTEHAEIKEMMRGIVRLGDNRKRKTGAL |
| | TLQPLTRVLDEIDTSNLAGLRDHTLLLLMFSGALRRSEAARIEVSDLDFVGQGIRLRLKPSKHQLHETEI |
| | ALIPGKQHCPVSALSRWLKASRLSQGPLFRRMTRWGQLTADPLGPQGINLMIKRRTGQAIDDLYVSGHSL |
| | RRGFITSAVTAGKPMNKIIEVTRHKDMRTLQEYFDDAHKFSDHALEGLL |
| 1438 | WP_042638308.1 |
| | MQAVFDPQLNSRARRFLRSAKADSTLNAYEADTRIFVYWCQLQQLDPLQTSHHDIMNFLADQADGILADW |
| | VWLDKQEGKGELRNGEPRKPATLVRRLAGIRYAFKQKGIHPMPTEHAEIKEMMRGIVRLGDNRKRKTGAL |
| | TLQPLIRVLDDIDTSNLAGLRDHTLLLLMFSGALRRSEAARIEVSDLDFVGQGIRLRLKPSKHQLHETEI |
| | ALIPGKQYCPVSALANWLKKSRIGEGPLFRRMNRWGQLMPEPLGPQGINLMIKRRTGQVIDDLYVSGHSL |
| | RRGFITSAVTAGKPMNKIIEVTRHKDMRTLQEYFDDAHKFSDHALDGLL |
| 1439 | WP_046400708.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLARVLDEIDTSNLAGLRDHTLLLLMFSGALRRSEAARIEVSDLDFVGQGIRLRLKPSKHQLHETEI |
| | ALIPGKQYCPVSALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDYAHKFSDHALDGLL |
| 1440 | ARW82171.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLARVLDEIDTSNLAGLRDHTLLLLMFSGALRRSEAARIEVSDLDFVGQGIRLRLKPSKHQLHETEI |
| | ALIPGKQYCPVSALHTWLKKSRIGEGALFRRMNRWGQLMSDPLGPQGINLMIKRRTGQVIDDLHVSGHSL |
| | RRGFITSAVTAGKPMNKIIEVTRHKDMRTLQEYFDDAHKFSDHALDGLL |
| 1441 | WP_042467353.1 |
| | MQTVFDPQLNSRARRFLRSAKAVSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLGRVLDEIDTSNLAGLRDHTLLLLMFSGALRRSEAARIEVSDLDFVGQGIRLRLKPSKHQLHETEI |
| | ALIPGKQYCPVSALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1442 | WP_051163765.1 |
| | MQTVFDPQLNSRARRFLRSAKAVSTLNAYQADTRIFVFWCQLHWLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLGRVLDEIDTSNLAGLRDHTLLLLMFSGALCRSEAARIEVSDLDFVGQGIRLRLKPSKHQLHETEI |
| | ALIPGKQYCPVSALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1443 | KOG94732.1 |
| | MQSVFDPQLNSRARRFLRSAKADSTLNAYQADTRIFVSWCQLHGLDPLQTTHHDIMNFLADQADGILANW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTSNLAGLRDYTLLLLMFSGALRRSEAARIEVNDVQFVGQGIRLCLKPSKHQLHESEI |
| | ALIPGTRYCPVSALQQWLKKSRIAEGPLFRRMNRWGQLMTDPLGPQGINLMIKRRTGQAIDDLHVSGHSL |
| | RRGFITSAVTAGKPMNKIIEVTRHKDMRTLQEYFDDAHKFSDHALDGLL |
| 1444 | EKB19089.1 |
| | MRSAKADSTLNAYQADTRIFVFWCQLHGLDPLQTTHHDIMNFLADQADGVLADWIWLDKEEGKGELRNGE |
| | PRKPATLVRRLAGIRYAFKQKGIHPMPTEHPEIKEMMRGIVRLGDNRKHKTGALTLQPLTQVLDGIDTGD |
| | LAGLRDHTLLLLMFSGALRRSEAARIEVSDLDFVGQGIRLRLKPSKHQLHETEIALIPGKQYCPVSALQK |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1445 | EKB18370.1 |
| | MRSAKADSTLNAYQADTRIFVFWCQLHGLDPLQTTHHDIMNFLADQADGVLADWVWLDKEEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFMGQGIRLRLKPSKHQLHETEIALIPGKHYCPVSALQK |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1446 | WP_082032588.1 |
| | MRSAKADSTLNAYQADTRIFVFWCQLHGLDPLQTTHHDIMNFLADQADGVLADWIWLDKEEGKGELRNGE |
| | PRKPATLVRRLAGIRYAFKQKGIHPMPTEHPEIKEMMRGIVRLGDNRKRKTGALTLQPLTQVLDGIDTHD |
| | LAGLRDHTLLLLMFSGALRRSEAARIEVSDLDFVGKGIRLRLKPSKHQLHETEIALIPGKHHCPVSALQK |
| | WLHKSRISEGALFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1447 | AEB50024.1 |
| | MRSAKADSTLNAYQADTRIFVFWCQLHGLDPLQTTHHDIMNFLADQADGVLANWVWLDKEEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFVGQGIRLRLKPSKHQLHETEIALIPGKQYCPVSALQK |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1448 | EQC05143.1 |
| | MRSAKADSTLNAYQADTRIFVFWCQLHGLDPLQTTHHDIMNFLADQADGILADWVWLDKEEGKGELRNGE |
| | PRKPATLVRRLAGIRYAFKQKGIHPMPTEHAEIKEMMRGIVRLGDNRKRKTGALTLQPLARVLDEIDTSN |
| | LAGLRDHTLLLLMFSGALRRSEAARIEVSDLDFVGQGIRLRLKPSKHQLHETEIALIPGKQYCPVSALHT |
| | WLKKSRIGEGALFRRMNRWGQLMSEPLGPQGINLMIKRRTGQAIDDLYVSGHSLRRGFITSAVTAGKPMN |
| | KIIEVTRHKDMRTLQEYFDYAHKFSDHALDGLL |
| 1449 | RAJ07841.1 |
| | MRSAKADSTLNAYQADTRIFVFWCQLHGLDPLQTTHHDIMNFLADQADGILADWVWLDKEEGKGELRNGE |
| | PRKPATLVRRLAGIRYAFKQKSIHPMPTEHAEIKEMMRGIVRLGDNRKRKTGALTLQPLARVLDEIDTSN |
| | LAGLRDHTLLLLMFSGALRRSEAARIEVSDLDFVGQGIRLRLKPSKHQLHETEIALIPGKQYCPVSALHT |
| | WLKKSRIGEGALFRRMNRWGQLMSDPLGPQGINLMIKRRTGQAIDDLYVSGHSLRRGFITSAVTAGKPMN |
| | KIIEVTRHKDMRTLQEYFDDAHKFSDHALDGLL |
| 1450 | WP_113739560.1 |
| | MAFPTLSNPAHQSLQTVFDAQLNSRARRFLRSAKADSTLNAYQADTRIFVFWCQLHRLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTHDLAGLRDHTLLLLMFSGALRRSEAARIEVTDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1451 | WP_061520510.1 |
| | MAENVNFFLKLFVEYLQIEKNYSQYTIVNYVNSIEEFEMFLHTQNINGMKEAAYHDVRIFLTEAYEKGLS |
| | RKTISKKISALRSFYKFLMREKLVEENPFQLVHLPKQEKRIPKFLYQKELEELFAVSDKSQPSGMRDQAL |
| | LELLYATGMRVSECCLLTVSDLDLFMDTVLVHGKGKKQRYIPFGSYAREALELYINSGRQCLLEKAKEPH |
| | DVLFVNQRGGPLTARGIRYILSGLVKKASGTLHIHPHMLRHTFATHLLNEGADLRSVQELLGHSNLSSTQ |
| | IYTHVSKEMLRNTYMSHHPRAFKEN |
| 1452 | WP_006951358.1 |
| | MIIKRNIIFTLESRKKDGILIIENVPIRMRVNFASKRIEFTTGYRIDAAKWDADKQRVKNGCSNKLKQSA |
| | SEINASLLGYYTKIQEIFKKFEVKEIMPTQEQIKEAFNALHKPIKEEVKPKKSTPNAFYKVFNEFVRDCG |
| | RQNDWTDSTYEKFAAVKNHLMNFHDELTFDFFDEKGLNDYVTYLRDVKEMRNSTIGKQLSFLKWFLRWAF |
| | KKGIHQNNAYDSYKPKLKSTQKKIIFLTWEELNRLREFEIPTSKQALDRVRDVFLFQCFTGLRYSDVFNL |
| | RRSDIKGDHIEVTTVKTSDSLIIELNNHSKAILDKYKDVAFEDDKVLPVITNQKMNDYLKELAELAGIDE |
| | PVRQTYYRGNERIDEVTPKYALLGTHAGRRTFICNALALGIPPQVVMKWTGHSDYKAMKPYIDIADDIKA |
| | NAMSKFNQL |
| 1453 | WP_040065515.1 |
| | MAYPSLSNPAHQSLQTVFDAQLNSRARRFLRSAKADSTLNAYQADTRIFVFWCQLHGLDPLQTTHHDIMN |
| | FLADQADGVLADWIWLDKEEGKGELRNGEPRKPATLVRRLAGIRYAFKQKGIHPMPTEHPEIKEMMRGIV |
| | RLGDNRKHKTGALTLQPLTQVLDGIDTGDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1454 | WP_101531573.1 |
| | MAYPTLSNPAHQSLQTVFDAQLNSRARRFLRSAKADSTLNAYQADTRIFVFWCQLHGLDPLQTTHHDIMN |
| | FLADQADGVLADWVWLDKEEGNGELRNGEPRKPATLVRRLAGIRYAFRQKGIHPMPTEHPEIKEMMRGIV |
| | RLGDNRKRKTGALTLQPLTQVLDGIDTGDLAGLRDHTLLLLMFSGALRRSEAARIEVSDLDFVGQGLRLR |
| | LKPSKHQLHETEIALILGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1455 | WP_041235050.1 |
| | MAYPSLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALILGKHYCPVSALQKWLHKSRISEGPLFRRMNRWGQLLAEPLGPQGINLMIKRRTG |
| | QTIDDLYVSGHSLRRGFITSAVTAGKPMNKIIEVTRHKDMRTLQEYFDDAHKFSDHALDGLL |
| 1456 | WP_082038647.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLTQEPLGPQGINLMIKRRTG |
| | QAIDDLQVSGHSLRRGFITSAVTAGKPMNKIIEVTRHKDIRTLQEYFDDAHKFSDHALDGLL |
| 1457 | WP_108588231.1 |
| | MAYPSLSNPAHQSLQTVFDAQLNSRARRFLRSAKADSTLNAYQADTRIFVFWCQLHGLDPLQTTHHDIMN |
| | FLADQADGVLADWIWLDKEEGKGELRNGEPRKPATLVRRLAGIRYAFKQKGIHPMPTEHPEIKEMMRGIV |
| | RLGDNRKHKTSALTLQPLTQVLDGIDTGDLAGLRDHTLLLLMFSGALRRSEAARIEVSDLDFMGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1458 | KRV94096.1 |
| | MAYPTLSNPAHQSLQTVFDAQLNSRARRFLRSAKADSTLNAYQTDTRIFVFWCQLHELEPLKTTHHDIMN |
| | FLADQADGVLADWVWLDKDEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1459 | WP_099359435.1 |
| | MNYPRILNPVQQPLQSVFDPQLNSRARRFLRSAKADATLNAYQADTRIFVFWCQLHGLDPLQTTHHDIMN |
| | FLADQADGILADWIWLDKEEGKGELRNGEPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1460 | WP_120414255.1 |
| | MTYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTNDLAGLRDHTLILLMFSGALRRSEAARIEVSDLDFMGQGIRLR |
| | LKPSKHQLHETEIALIPGKHYCPVSALQKWLHKSRISEGALFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1461 | WP_101347286.1 |
| | MAFPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGINPMPTEHPEIKEMMRGIV |
| | RLGDNRKRKTGALTLQPLTRVLDGIDTTNLAGLRDHTLLLLMFSGALRRSEAARIEVSDLDFVGQGIRLR |
| | LKPSKHQLHETEIALIPGKHHCPVSALQHWLRKSRISEGHLFRRMNRWGQLMTDPLGPQGINLMIKRRTG |
| | QAIDDLYVSGHSLRRGFITSAVTAGKPMNKIIEVTRHKDMRTLQEYFDDAHKFSDHALDGLL |
| 1462 | WP_106843696.1 |
| | MAYPSLSNPAHQSLQTVFDAQLNSRARRFLRSAKADSTLNAYQADTRIFVFWCQLHGIDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALIPGKHYCPVSALQNWLRKSRISEGPLFRRMNRWGQLMAEPLGPQGINLMIKRRTG |
| | QAIDDLYVSGHSLRRGFITSAVTAGKPMNKIIEVTRHKDMRTLQEYFDDAHKFSDHALDGLL |
| 1463 | WP_124242906.1 |
| | MSHPSISGSAHQSLQTVFDAQLNSRARRFLRSAKADSTLNAYQTDTRIFVFWCQLHGLDPLQTTHHDIMN |
| | FLADQADGVLANWVWLNKEEGRGELRNGEPRKPATLVRRLAGIRYAFKQKGIHPMPTEHPEIKEMMRGIV |
| | RLGDNRKRKTGALTLQPLTQVLDGIDTNDLAGLRDHTLLLLMFSGALRRSEAARIEVSDLDFVGQGIRLR |
| | LKPSKHQLHETEIALIPGKHYCPVAAIGNWLKKSRINEGPLFRRMNRWGQLTPDPLGPQGINLMIKRRTG |
| | QAIDDLYVSGHSLRRGFITSAVTAGKPMNKIIEVTRHKDMRTLQEYFDDAHKFSDHALDGLL |
| 1464 | WP_041202700.1 |
| | MAYPSLSNPAHQSLQTVFDAQLNSRARRFLRSAKADSTLNAYQADTRIFVFWCQLHGLDPLQTTHHDIMN |
| | FLADQADGVLANWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALIPGKQYCPVSALQNWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1465 | WP_123173050.1 |
| | MTYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTRDLAGLRDHTLLLLMFSGALRRSEAARIEVSDLDFVGQGIRLR |
| | LKPSKHQLHETEIALIPGKHYCPVSALQNWLSKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALNGLL |
| 1466 | WP_107682950.1 |
| | MAYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTGDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1467 | WP_128821547.1 |
| | MAYPTLSSPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTHDLAGLRDHTLLLLMFSGALRRSEAARIEVSDLDFVGQGIRLR |
| | LKPSKHQLHETEIALIPGKHYCPVSALQNWLRKSRINEGPLFRRMNRWGQLMAEPLGPQGINLMIKRRTG |
| | QAIDDLYVSGHSLRRGFITSAVTAGKPMNKIIEVTRHKDMRTLQEYFDDAHKFSDHALDGLL |
| 1468 | WP_082180660.1 |
| | MNYPRLQNRVQQSLQSIFDPQLNSRARRFLRSAKADSTLNAYQADTRIFVSWCQLHGLDPLQTTHHDIMN |
| | FLADQADGVLANWVWLDKEEGKGELRNGEPRKPATLVRRLAGIRYAFKQKGIHPMPTEHAEIKEMMRGIV |
| | RLGDNRKRKTGALTLKPLACVLDEIDTCNLAGLRDYTLLLLMFSGALRRSEAARIEVNDVQFVGQGIRLR |
| | LKPSKHQLHESEIALIPGTRYCPVAALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1469 | WP_082029942.1 |
| | MAYPTISPPAHQSLQTVFDPALNSRARRFLRSAKADSTLNAYQADTRIFVFWCQLHGLDPLQTSHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLTRVLDEIDTSTLAGLRDHTLLLLMFSGALRRSEAARIEVSDLDFVGQGIRLR |
| | LKPSKHQLHETEIALIPGKHYCPVTALGRWLKASRISQGPLFRRMTRWGQLTADPLGPQGINLMIKRRTG |
| | QVIDDLYVSGHSLRRGFITSAVTAGKPMNKIIEVTRHKDMRTLQEYFDDAHKFSDHALAGLL |
| 1470 | WP_081013237.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRIHEGALFRRMNRWGQLTQEPLGPQGINLMIKRRTG |
| | QAIDDLQVSGHSLRRGFITSAVTAGKPMNKIIEVTRHKDIRTLQEYFDDAHKFSDHALDGLL |
| 1471 | WP_024941785.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDLDFVGQGIRLR |
| | LKPSKHQLHETEIALVPGKQYCPVSALARWLKQSRIHEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1472 | WP_065017596.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLNFVGQGIRLR |
| | LKPSKHQLHETEIALVPGKQYCPVSALARWLKQSRIHEGALFRRMNRWGQLTQEPLGPQGINLMIKRRTG |
| | QAIDDLQVSGHSLRRGFITSAVTAGKPMNKIIEVTRHKDIRTLQEYFDDAHKFSDHALDGLL |
| 1473 | WP_042889028.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLKPLACVLDEIDTSTLAGLRDYTLLLLMFSGALRRSEAARIEVDDLDFVGQGIRLR |
| | LKPSKHQLHETEIALVPGKQYCPVSALARWLKQSRIHEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1474 | WP_111910613.1 |
| | MAYPTISQPVOQSLQTVFDPALNSRARRFLRSAKADSTLNAYQADTRIFVFWCQLHGLDPLQTSHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSTLAGLRDHTLLLLMFSGALRRSEAARIEVGDLDFVGQGIRLR |
| | LKPSKHQLHETEIALIPGKQYCPVTALSRWLKASRISQGPLFRRMTRWGQLTAEPLGPQGINLMIKRRTG |
| | QVIDDLYVSGHSLRRGFITSAVTAGKPMNKIIEVTRHKDMRTLQEYFDDAHKFSDHALAGLL |
| 1475 | WP_126881846.1 |
| | MNYPRISNPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLQPLACVLDEIDTGNLAGLRDYTLLLLMFSGALRRSEAARIEVDDLDFVGQGIRLR |
| | LKPSKHQLHETEIALVPGKQYCPVSALARWLKQSRIHEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1476 | WP_017779021.1 |
| | MNYPRISSPVOQPLQSVFDPQLNSRARRFLRSAKADSTLNAYQADTRIFVFWCQLHGLDPLQTTHHDIMN |
| | FLADQADGILADWVWLDKEEGKGELRNGDPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRIHEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1477 | WP_080768865.1 |
| | MNYPHIQAQTQQALQSVFDPQLNSRARRFLRSAKADSTLNAYQADTRIFVFWCQLHGLDPLQTTHHDIMN |
| | FLADQAEGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLVGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDNAHKFSDHALDGLL |
| 1478 | WP_080973138.1 |
| | MNYPHIQPQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLF |
| 1479 | WP_024944768.1 |
| | MNYPHIQAQTQQALQSVFDPQLNSRAKRFLRSAKADSTLNAYQADTRIFVFWCQLHGLDPLQTTHHDIMN |
| | FLADQADGILADWIWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1480 | WP_106552588.1 |
| | MNYPHIQAQAQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1481 | WP_113995002.1 |
| | MNYTHIQAQTQQALQSVFDPQLNNRARRFLRSAKADSTLNAYQADTRIFVCWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1482 | WP_130632356.1 |
| | MNYPHIQAQTKQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1483 | WP_113721656.1 |
| | MAYPTVSPPVYQSLQTVFDPLLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLGRVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDDAHKFSDHALDGLL |
| 1484 | WP_088846217.1 |
| | MAYPTVSPPVYQSLQTVFDPQLNSRARRFLRSAKAVSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLGRVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDDAHKFSDHGVLGIFPSKVT |
| 1485 | WP_076360755.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLASVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMPEPLGPQGINLMIKRRTG |
| | QAIDDLQVSGHSLRRGFITSAVTAGKPMNKIIEVTRHKDIRTLQEYFDDAHKFSDHALDGLL |
| 1486 | WP_131730694.1 |
| | MAYPTVSPPIYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLAKVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDDAHKFSDHALDGLL |
| 1487 | WP_103243980.1 |
| | MATTPAVTDMPDTGSAPAIRTAVTPQDDHNHAARHRVFLAAATSDNTRQAYRSAVKHYLDWGGVLPANEP |
| | AVIRYLVRYADTLNPRTLALRLTALSQWHVHQGFADPAATPTVRKTLAGIARTNGRPKKKAKALPIEDLE |
| | LIVANLASLGTLKAARDNALLQVGFFGGFRRSELVGIKVDHITWEAQGITLTLPRSKTDQTGEGVAKAIP |
| | YSAGPCCPATALRTWLDAAGVASGPVFRSISKWGVVGADRLNPASVNTILAGAAQLAKLGYVPELSSHSL |
| | RRGMATSAHRAGAEFRDIKKQGGWRHDGTVQGYIEEAGLFEENAAGSLLRSRTRTSG |
| 1488 | WP_081304608.1 |
| | MNYQQ1QAQTHQALQSVFDPQLNSRARRFLRSAKADSTLNAYQADTRIFVFWCQLHGLDPLQTTHHHIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1489 | WP_118881229.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEERKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1490 | WP_029300882.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLAYVLDEIDTGNLAGLRDYTLLLLMFSGALRRSEAARIEVGDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1491 | WP_102988785.1 |
| | MNYPHIQAQTQQALQSVFDPQLNSRARRFLRSAKADSTLSAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1492 | WP_034523632.1 |
| | MAYPTVSPPVYQSLQTVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMSEPLGPQGINLMIKRRTG |
| | QAIDDLYVSGHSLRRGFITSAVTAGKPMNKIIEVTRHKDMRTLQEYFDYAHKFSDHALDGLL |
| 1493 | WP_011706113.1 |
| | MNYPDIQAQTQQALQSVFDPQLNSRARRFLRSAKADSTLNAYQADTRIFVFWCQLHGLDPLQTTHHHIMN |
| | FLADQADGILADWVWLDKEEGRGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1494 | WP_081086191.1 |
| | MNYPHIQAQTQQALQSVFDPQLNSRARRFLRSAKADSTLNAYQADTRIFVFWCQLHGLDPLQTTHHHIMN |
| | FLADQADGILADWVWLDKEEGRGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIAMVPGKQYCPVSALARWLKQSRISEGALFRRMNRWGQLMQEPLGPQGINLMIKRRTG |
| | QAIDDLQVSGHSLRRGFITSAVTAGKPMNKIIEVTRHKDIRTLQEYFDDAHKFSDHALDGLL |
| 1495 | WP_045789855.1 |
| | MNYPHIQAQTQQALQSVFDPQLNSRARRFLRSAKADSTLNAYQADTRIFVFWCQLHGLDPLLTTHHHIMN |
| | FLADQADGILADWIWLDKEEGKGELRNGEPRKPATLVRRLAGIRYAFKQKGIHPMPTEHAEIKEMMRGIV |
| | RLGDNRKRKTGALTLKPLACVLDEIDTGNLAGLRDYTLLLLMFSGALRRSEAARIEVDDLDFVGQGIRLR |
| | LKPSKHQLHETEIALVPGKQYCPVSALARWLKQSRISEGALFRRMNRWGQLMPEPLGPQGINLMIKRRTG |
| | QAIDDLQVSGHSLRRGFITSAVTAGKPMNKIIEVTRHKDIRTLQEYFDDAHKFSDHALDGLL |
| 1496 | WP_101617448.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDEIDIGNLAGLRDYTLLLLMFSGALRRSEAARIEVDDLDFVGQGIRLR |
| | LKPSKHQLHETEIALVPGKQYCPVSALALWLKQSRISEGALFRRMNRWGQLMQEPLGPQGINLMIKRRTG |
| | QAIDDLQVSGHSLRRGFITSAVTAGKPMNKIIEITRHKDIRTLQEYFDDAHKFSDHALDGLL |
| 1497 | WP_099993215.1 |
| | MAYPTVSPPVYQSLQTVFDPQLNSRARRFLRSAKADSTLNAYQADTRIFVFWCQLHMLDPLQTTHHDIMN |
| | FLADQADGILADWVWLDKEAGKGELRNGEPRKPATLVRRLAGIRYAFKQKGIHPMPTEHAEIKEMMRGIV |
| | RLGDNRKRKTGALTLQPLARVLDEIDTSNLAGLRDHTLLLLMFSGALRRSEAARIEVSDLDFVGQGIRLR |
| | LKPSKHQLHETEIALIPGKQYCPVSALHTWLKKSRIGEGALFRRMNRWGQLMPDPLGPQGINLMIKRRTG |
| | QAIDDLYVSGHSLRRGFITSAVTAGKPMNKIIEVTRHKDMRTLQEYFDDAHKFSDHALDGLL |
| 1498 | WP_104455933.1 |
| | MNYPRLQNPVOQSLOSVFDPQLNSRARRFLRSAKADSTLNAYQADTRIFVSWCQLHGLDPLQTTHHDIMN |
| | FLADQADGILANWVWLDKEEGKGELRNGKPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGTRYCPVSALQQWLKKSRIAEGPLFRRMNRWGQLMTDPLGPQGINLMIKRRTD |
| | QAIDDLHVSGHSLRRGFITSAVTAGKPMNKIIEVTRHKDMRTLQEYFDDAHKFSDHALDGLL |
| 1499 | WP_042863872.1 |
| | MNYPSLQNPVOQSLOSVFDPQLNSRARRFLRSAKADSTLNAYQADTRIFVSWCQLHGLDPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVNDVQFVGQGIRLR |
| | LKPSKHQLHESEIALIPGTRYCPVSALQQWLKKSRIAEGPLFRRMNRWGQLMTDPLGPQGINLMIKRRTG |
| | QAIDDLHVSGHSLRRGFITSAVTAGKPMNKIIEVTRHKDMRTLQEYFDDAHKFSDHALDGLL |
| 1500 | WP_041205782.1 |
| | MNYPSLQNPVOQSLOSVFDPQLNSRARRFLRSAKADSTLNAYQADTRIFVSWCQLHGLEPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGTRYCPVSALQQWLKRSRIAEGPLFRRMNRWGQLMADPLGPQGINLMIKRRTG |
| | QGIDDLHVSGHSLRRGFITSAVTAGKPMNKIIEVTRHKDMRTLQEYFDDAHKFSDHALDGLL |
| 1501 | WP_043152710.1 |
| | MNYPRLQNPVOQSLOSVFDPQLNSRARRFLRSAKADSTLNAYQADTRIFVSWCQLHGLEPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDAIDTSNLAGLRDYTLLLLMFSGALRRSEAARIEVNDVQFVGQGIRLR |
| | LKPSKHQLHESEIALIPGIRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1502 | WP_103858936.1 |
| | MNYPRLQNPVOQSLOSVFDPQLNSRARRFLRSAKADSTLNAYQADTRIFVSWCQLHGLDPLQTTHHDIMN |
| | FLADQADGVLANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGIRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1503 | WP_124239332.1 |
| | MAYPTFSNPAHQSLQTIFDAQLNSRARRFLRSAKADSTLNAYQADTRIFVFWCQLHGLDPLQITHHDIMN |
| | FLADQADGVLADWVWLDKEEGKGELRNGEPRKPATLVRRLAGIRYAFQQKGIHPMPTEHPEIKEMMRGIV |
| | RLGDNRKRKTGALTLQPLARVLSGIDTSTLAGLRDYTLLLLMFSGALRRSEAARIEVSDLDFVGQGIRLR |
| | LKPSKHQLHETEIALVPGKQYCPVQGLQHWLEKSRIKEGALFRRMNRWGQLTEEPLGPQGINQMIKRRTG |
| | QAIDDLYVSGHSLRRGFITSAVTAGKPMNKIIEVTRHKDMRTLQEYFDDANKFSDHALDGLL |
| 1504 | WP_103261885.1 |
| | MNYPSLQNPVOQSLOSVFDPQLNSRARRFLRSAKADSTLNAYQADTRIFVSWCQLHGLDPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTCNLAGLRDYTLLLLMFSGALRRSEAARIEVNDVQFVGQGIRLR |
| | LKPSKHQLHESEIALIPGTRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1505 | WP_103260130.1 |
| | MNYPRLQNPVOQSLOSVFDPQLNSRARRFLRSAKADSTLNAYQADTRIFVSWCQLHGLDPLQTTHHDIMN |
| | FLADQADGVLANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGTRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1506 | WP_111809297.1 |
| | MAYPTVSPPVYQSLQTVFDPQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLIQVLNGIDTHDLAGLRDHTLLLLMFSGALRRSEAARIEVSDLDFVGQGIRLR |
| | LKPSKHQLHETEIALIPGKHYCPVSALQNWLHKSRISEGPLFRRMNRWGQLMAEPLGPQGINLMIKRRTG |
| | QAIDDLYVSGHSLRRGFITSAVTAGKPMNKIIEVTRHKDMRTLQEYFDDAHKFSDHALDGLL |
| 1507 | WP_081331871.1 |
| | MNYPRLQNPVOQSLOSVFDPQLNSRARRFLRSAKADSTLNAYQADTRIFVSWCQLHGLEPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVDDVQFVGQGIRLR |
| | LKPSKHQLHESEIALIPGTRYCPVSALQQWLKRSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1508 | WP_041215162.1 |
| | MNYPRLQNPVOQSLOSVFDPQLNSRARRFLRSAKADSTLNAYQADTRIFVSWCQLHGLDPLQTTHHDIMN |
| | FLADQADGILANWVWLDKEEGKGELRNGEPRKPATLVRRLAGIRYAFKQKGIHPMPTEHAEIKEMMRGIV |
| | RLGDNRKRKTGALTLKPLACVLDEIDTSNLAGLRDYTLLLLMFSGALRRSEAARIEVNDVQFVGQGIRLR |
| | LKPSKHQLHESEIALIPGTRYCPVSALQQWLKKSRIAEGPLFRRMNRWGQLMADPLGPQGINLMIKRRTG |
| | QAIDDLHVSGHSLRRGFITSAVTAGKPMNKIIEVTRHKDMRTLQEYFDDAHKFSDHALDGLL |
| 1509 | WP_126623323.1 |
| | MAYPTVSPPVHQSLQAVFDPALNNRARRFLRSAKADSTLNAYQADTRIFVFWCQLHGLDPLQTSHHDIMN |
| | FLADQADGILADWVWLDREEGKGELRNGEPRKPATLVRRLAGIRYAFKQKGIHPMPTEHAEIKEMMRGIV |
| | RLGDNRKRKTGALTLQPLTRVLDEIDTSNLAGLRDHTLLLLMFSGALRRSEAARIEVSDLDFVGQGIRLR |
| | LKPSKHQLHETEIALIPGKQHCPVSALSRWLKASRLSQGPLFRRMTRWGQLTADPLGPQGINLMIKRRTG |
| | QAIDDLYVSGHSLRRGFITSAVTAGKPMNKIIEVTRHKDMRTLQEYFDDAHKFSDHALEGLL |
| 1510 | WP_050490004.1 |
| | MQTVFDAQLNSRARRFLRSAKADSTLNAYQADTRIFVFWCQLHGLDPLQTTHHDIMNFLADQADGVLADW |
| | VWLNKEEGRGELRNGEPRKPATLVRRLAGIRYAFKQKGIHPMPTEHPEIKEMMRGIVRLGDNRKRKTGAL |
| | TLQPLTQVLDGIDTNDLAGLRDHTLLLLMFSGALRRSEAARIEMSDLDFVGQGIRLRLKPSKHQLHETEI |
| | ALIPGKHYCPVAAIGNWLKKSRINEGPLFRRMNRWGQLTPDPLGPQGINLMIKRRTGQAIDGLYVSGHSL |
| | RRGFITSAVTAGKPMNKIIEVTRHKDMRTLQEYFDDAHKFSDHALDGLL |
| 1511 | WP_042030957.1 |
| | MQTVFDAQLNSRARRFLRSAKADSTLNAYQADTRIFVFWCQLHGLDPLQTTHHDIMNFLADQADGVLADW |
| | VWLNKEEGRGELRNGEPRKPATLVRRLAGIRYAFKQKGIHPMPTEHPEIKEMMRGIVRLGDNRKRKTGAL |
| | TLQPLTQVLDGIDTNNLAGLRDHTLLLLMFSGALRRSEAARIEVSDLDFVGQGIRLRLKPSKHQLHETEI |
| | ALIPGKHYCPVAAIGNWLKKSRINEGPLFRRMNRWGQLTPDPLGPQGINLMIKRRTGQAIDDLYVSGHSL |
| | RRGFITSAVTAGKPMNKIIEVTRHKDMRTLQEYFDDAHKFSDHALDGLL |
| 1512 | WP_042083230.1 |
| | MQTVFDTQLNSRARRFLRSAKADSTLNAYQADTRIFVCWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTGDLAGLRDHTLLLLMFSGALRRSEAARIEVSDLDFVGQGIRLRLKPSKHQLHETEI |
| | ALIPGKHYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1513 | WP_064340028.1 |
| | MQTVFDAQLNSRARRFLRSAKADSTLNAYQADTRIFVFWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDIGDLAGLRDHTLLLLMFSGALRRSEAARIEVSDLDFVGQGIRLRLKPSKHQLHETEI |
| | ALIPGKHYCPVSALQNWLRKSRISEGPLFRRMNRWGQLMTEPLGPQGINLMIKRRTGQAIDDLYVSGHSL |
| | RRGFITSAVTAGKPMNKIIEVTRHKDMRTLQEYFDDAHKFSDHALDGLL |
| 1514 | WP_041980781.1 |
| | MQTVFDAQLNSRARRFLRSAKADSTLNAYQADTRIFVFWCQLHGLDPLQTTHHDIMNFLADQADGVLANW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTGDLAGLRDHTLLLLMFSGALRRSEAARIEVSDLDFVGQGIRLRLKPSKHQLHETEI |
| | ALIPGKQYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1515 | WP_042655814.1 |
| | MQTVFDAQLNSRARRFLRSAKADSTLNAYQADTRIFVFWCQLHGLDPLQTTHHDIMNFLADQADGVLADW |
| | VWLDKEEGKGELRNGEPRKPATLVRRLAGIRYAFKQKGIHPMPTEHPEIKEMMRGIVRLGDNRKRKTGAL |
| | TLQPLTQVLDGIDTHDLAGLRDHTLLLLMFSGALRRSEAARIEVSDLDFVGQGIRLRLKPSKHQLHETEI |
| | ALIPGKHYCPVSALQNWLRKSRINEGPLFRRMNRWGQLMAEPLGPQGINLMIKRRTGQAIDDLYVSGHSL |
| | RRGFITSAVTAGKPMNKIIEVTRHKDMRTLQEYFDDAHKFSDHALDGLL |
| 1516 | WP_052447116.1 |
| | MQTVFDAQLNSRARRFLRSAKADSTLNAYQADTRIFVFWCQLHGLDPLQTTHHDIMNFLADQADGVLADW |
| | IWLDKEEGKGELRNGEPRKPATLVRRLAGIRYAFKQKGIHPMPTEHPEIKEMMRGIVRLGDNRKHKTGAL |
| | TLQPLTQVLDGIDTGDLAGLRDHTLLLLMFSGALRRSEAARIEVSDLDFVGQGIRLRLKPSKHQLHETEI |
| | ALIPGKHYCPVSALQKWLHKSRISEGPLFRRMNRWGQLMAEPLGPQGINLMIKRRTGQTIDDLYVSGHSL |
| | RRGFITSAVTAGKPMNKIIEVTRHKDMRTLQEYFDDAHKFSDHALDGLL |
| 1517 | PHS84353.1 |
| | MQSVFDPQLNSRARRFLRSAKADATLNAYQADTRIFVFWCQLHGLDPLQTTHHDIMNFLADQADGILADW |
| | IWLDKEEGKGELRNGEPRKPATLVRRLAGIRYAFKQKGIHPMPTEHAEIKEMMRGIVRLGDNRKRKTGAL |
| | TLQPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1518 | WP_042037844.1 |
| | MQTVFDAQLNSRARRFLRGAKADSTLNAYQADTRIFVFWCLLHGLDPLQTTHHDIMNFLADQADGVLADW |
| | VWLDKEEGKGELRNGEPRKPATLVRRLAGIRYAFKQKGIHPMPTEHAEIKEMMRGIVRLGDNRKRKTGAL |
| | TLQPLTQVLDGIDTTALAGLRDHTLLLLMFSGALRRSEAARIEVSDLDFVGQGIRLRLKPSKHQLHETEI |
| | ALIPGKHYCPVSALQNWLRKSRISDGPLFRRMNRWGQLMTEPLGPQGINLMIKRRTGQTIDDLYVSGHSL |
| | RRGFITSAVTAGKPMNKIIEVTRHKDMRTLQEYFDDAHKFSDHALDGLL |
| 1519 | OEG05223.1 |
| | MQSVFDPQLNSRARRFLRSAKADSTLNAYQADTRIFVSWCQLHGLEPLQTTHHDIMNFLADQADGILANW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTSNLAGLRDYTLLLLMFSGALRRSEAARIEVDDVQFVGQGIRLRLKPSKHQLHESEI |
| | ALIPGTRYCPVSALQQWLKRSRIAEGPLFRRMNRWGQLMADPLGPQGINLMIKRRTGQAIDDLHVSGHSL |
| | RRGFITSAVTAGKPMNKIIEVTRHKDMRTLQEYFDDAHKFSDHALDGLL |
| 1520 | KLV47629.1 |
| | MQSIFDPQLNSRARRFLRSAKADSTLNAYQADTRIFVSWCQLHGLDPLQTTHHDIMNFLADQADGVLANW |
| | VWLDKEEGKGELRNGEPRKPATLVRRLAGIRYAFKQKGIHPMPTEHAEIKEMMRGIVRLGDNRKRKTGAL |
| | TLKPLACVLDEIDTCNLAGLRDYTLLLLMFSGALRRSEAARIEVNDVQFVGQGIRLRLKPSKHQLHESEI |
| | ALIPGTRYCPVAALQQWLKKSRIAEGPLFRRMNRWGQLMADPLGPQGINLMIKRRTGQAIDDLHVSGHSL |
| | RRGFITSAVTAGKPMNKIIEVTRHKDMRTLQEYFDDAHKFSDHALDGLL |
| 1521 | AXV34415.1 |
| | MQSVFDPQLNSRARRFLRSAKADSTLNAYQADTRIFVFWCQLHGLDPLQTTHHDIMNFLADQADGILADW |
| | VWLDKEERKGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1522 | OCA59831.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLGRVLDEIDTSNLAGLRDHTLLLLMFSGALRRSEAARIEVSDLDFVGQGIRLRLKPSKHQLHETEI |
| | ALIPGKQYCPISALHTWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1523 | SUU28072.1 |
| | MQSVFDPQLNSRARRFLRSAKADSTLNAYQADTRIFVFWCQLHGLDPLQTTHHHIMNFLADQADGILADW |
| | VWLDKEEGRGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | ALVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1524 | KWR69035.1 |
| | MQSVFDPQLNSRARRFLRSAKADSTLNAYQADTRIFVFWCQLHGLDPLQTTHHHIMNFLADQADGILADW |
| | VWLDKEEGRGELRNGEPRKPATLVRRLAGIRYAFKQKGIHPMPTEHAEIKEMMRGIVRLGDNRKRKTGAL |
| | TLKPLACVLDEIDTGNLAGLRDYTLLLLMFSGALRRSEAARIEVDDLDFVGQGIRLRLKPSKHQLHETEI |
| | AMVPGKQYCPVSALARWLKQSRISEGALFRRMNRWGQLMQEPLGPQGINLMIKRRTGQAIDDLQVSGHSL |
| | RRGFITSAVTAGKPMNKIIEVTRHKDIRTLQEYFDDAHKFSDHALDGLL |
| 1525 | WP_052449173.1 |
| | MQTVFDPQLNSRARRFLRSAKADSTLNAYQADTRIFVFWCQLHGLDPLQTTHHDIMNFLADQADGILADW |
| | VWLDKEEGKGELRNGEPRKPATLVRRLAGIRYAFKQKGIHPMPTEHAEIKEMMRGIVRLGDNRKRKTGAL |
| | TLQPLARVLDEIDTSSLAGLRDHTLLLLMFSGALRRSEAARIEVSDLDFVGQGIRLRLKPSKHQLHETEI |
| | ALIPGKQYCPVSALHSWLKKSRIGEGALFRRMNRWGQLMSEPLGPQGINLMIKRRTGQAIDDLYVSGHSL |
| | RRGFITSAVTAGKPMNKIIEVTRHKDMRTLQEYFDDAHKFSDHALDGLL |
| 1526 | WP_050717134.1 |
| | MQAVFDPQLNSRARRFLRSAKADSTLNAYEADTRIFVYWCHLQQLDPLQTTHHDIMNFLADQADGILADW |
| | VWLDKQEGKGELRNGEPRKPATLVRRLAGIRYAFKQKGVHPMPTEHAEIKEMMRGIVRLGDNRKRKTGAL |
| | TLQPLTQVLDEIDTNNLAGLRDHTLLLLMFSGALRRSEAARIEVSDLEFVGQGVRLRLKPSKHQLHETEI |
| | ALIPGKHHCPVRALQNWLKKSRISEGPLFRRMNRWGQLMPDPLGPQGINLMIKRRTGQVIDSLYVSGHSL |
| | RRGFITSAVTAGKPMNKIIEVTRHKDMRTLQEYFDDAHKFSDHALDGLL |
| 1527 | OJW69670.1 |
| | MQSIFDPQLNSRARRFLRSAKADSTLNAYQADTRIFVSWCQLHGLDPLQTTHHDIMNFLADQADGILANW |
| | VWLDKEEGKGELRNGEPRKPATLVRRLAGIRYAFKQKGIHPMPTEHAEIKEMMRGIVRLGNNRKRKTGAL |
| | TLKPLACVLDEIDTSNLAGLRDYTLLLLMFSGALRRSEAARIEVNDVQFVGQGIRLCLKPSKHQLHESEI |
| | ALIPGTRYCPVSALQQWLKKSRIAEGPLFRRMNRWGQLMTDPLGPQGINLMIKRRTGQAIDDLHVSGHSL |
| | RRGFITSAVTAGKPMNKIIEVTRHKDMRTLQEYFDDAHKFSDHALDGLL |
| 1528 | VEG96551.1 |
| | MFDPALNNRARRFLRSAKADSTLNAYQADTRIFVFWCQLHGLDPLQTSHHDIMNFLADQADGILADWVWL |
| | DREEGKGELRNGEPRKPATLVRRLAGIRYAFKQKGIHPMPTEHAEIKEMMRGIVRLGDNRKRKTGALTLQ |
| | PLTRVLDEIDTSNLAGLRDHTLLLLMFSGALRRSEAARIEVSDLDFVGQGIRLRLKPSKHQLHETEIALI |
| | PGKQHCPVSALSRWLKASRLSQGPLFRRMTRWGQLTADPLGPQGINLMIKRRTGQAIDDLYVSGHSLRRG |
| | FITSAVTAGKPMNKIIEVTRHKDMRTLQEYFDDAHKFSDHALEGLL |
| 1529 | WP_084202279.1 |
| | MRSAKADSTLNAYQADTRIFVFWCQLHGLDPLQTTHHDIMNFLADQADGVLADWVWLDKEEGKGELRNGE |
| | PRKPATLVRRLAGIRYAFKQKGIHPMPTEHPEIKEMMRGIVRLGDNRKRKTGALTLQPLTQVLDGIDISD |
| | LAGLRDHTLLLLMFSGALRRSEAARIEVSDLDFVGQGIRLRLKPSKHQLHETEIALIPGKHYCPVSALQN |
| | WLRKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1530 | WP_080741249.1 |
| | MRSAKADSTLNAYQADTRIFVFWCQLHGLDPLQTTHHDIMNFLADQADGVLADWVWLDKEEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFLGQGLRLRLKPSKHQLHETEIALIPGKHYCPVSALQN |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1531 | EKB22195.1 |
| | MRSAKADSTLNAYQADTRIFVFWCQLHGLDPLQTTHHDIMNFLADQADGVLADWVWLDKEEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFVGQGIRLRLKPSKHQLHETEIALILGKHYCPVSALQK |
| | WLHKSRISEGPLFRRMNRWGQLLAEPLGPQGINLMIKRRTGQTIDDLYVSGHSLRRGFITSAVTAGKPMN |
| | KIIEVTRHKDMRTLQEYFDDAHKFSDHALDGLL |
| 1532 | WP_081042909.1 |
| | MRSAKADSTLNAYQTDTRIFVFWCQLHELEPLKTTHHDIMNFLADQADGVLADWVWLDKDEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFVGQGIRLRLKPSKHQLHETEIALIPGKHYCPVSALQK |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1533 | EKB14410.1 |
| | MRSAKADSTLNAYQADTRIFVFWCQLHGLDPLQTTHHDIMNFLADQADGVLANWVWLDKEEGKGELRNGE |
| | PRKPATLVRRLAGIRYAFKQKGIHPMPTEHPEIKEMMRGIVRLGDNRKRKTGALTLQPLTQVLDGIDTGD |
| | LAGLRDHTLLLLMFSGALRRSEAARIEVSDLDFVGQGIRLRLKPSKHQLHETEIALIPGKQYCPVSALQN |
| | WLHKSRISEGPLFRRMNRWGQLMAEPLGPQGINLMIKRRTGQTIDDLYVSGHSLRRGFITSAVTAGKPMN |
| | KIIEVTRHKDMRTLQEYFDDAHKFSDHALDGLL |
| 1534 | ANT70015.1 |
| | MRSAKADSTLNAYQADTRIFVFWCQLHGLDPLQTTHHHIMNFLADQADGILADWVWLDKEEGKGELRNGE |
| | PRKPATLVRRLAGIRYAFKQKGIHPMPTEHAEIKEMMRGIVRLGDNRKRKTGALTLKPLACVLDEIDTGN |
| | LAGLRDYTLLLLMFSGALRRSEAARIEVDDLDFVGQGIRLRLKPSKHQLHETEIALVPGKQYCPVSALAR |
| | WLKQSRISEGALFRRMNRWGQLMQEPLGPQGINLMIKRRTGQAIDDLQVSGHSLRRGFITSAVTAGKPMN |
| | KIIEVTRHKDIRTLQEYFDDAHKFSDHALDGLL |
| 1535 | EHI53752.1 |
| | MRSAKAVSTLNAYQADTRIFVFWCQLHGLDPLQTTHHDIMNFLADQADGILADWVWLDKEEGKGELRNGE |
| | PRKPATLVRRLAGIRYAFKQKGIHPMPTEHAEIKEMMRGIVRLGDNRKRKTGALTLQPLGRVLDEIDTSN |
| | LAGLRDHTLLLLMFSGALRRSEAARIEVSDLDFVGQGIRLRLKPSKHQLHETEIALIPGKQYCPVSALHT |
| | WLKKSRIGEGALFRRMNRWGQLMSEPLGPQGINLMIKRRTGHRRSLCQWPQPATGIHHLGRHRRQAHEQD |
| | H |
| 1536 | WP_045972172.1 |
| | MAAELNKLSDKKLKNLHGKERDNIEFFADGAGLSAKASKVGGISWVFTYRLDGKKLNRLTIGRYPDMSLK |
| | LARDMRDKCRNWLASGKDPKLQFDLTMQESLKPVTVKEAMEYWIENYAKDSRENIDKHVSQLKKHIYPYI |
| | GNMALADCETRYWLQCFDRTKKTAPVGAGYILQMCKQALKFCRVRRFAISNALDDLTISDVGRKQSKGKR |
| | YLEDNELSQLWQSLNTNMYLPYYSNLLRILIVFGCRSQEARLSKWSEWDFDSMLWTVPKENSKSDDKIIR |
| | PIPECLKPFLEKLIYQNHKSGYLLGELKSPESVSQCGRNIWRRLEHGEEWSLHDLRRTFATKLNDMGIAP |
| | HIVEQLLGHALPGIMAIYNKSQYLPEKLDALNKWCERLDVLAGNYENVVILKAVQ |
| 1537 | WP_073614059.1 |
| | MQHLPAPIHHARDIAQLPVAIDYPAALALRQMSMVHDELPKYLLAPEVSALLHYVPDLRRKMLLATLWNT |
| | GARINEALALTRGDFSLTPPYPFVQLATLKQRTEKAARTAGRMPAGQQTHRLVPLSDTWYVSQLQTMVAT |
| | LKIPMERRNKRTGRTEKARIWEVTDRTVRTWIGEAVAAAAADGVTFSVPVTPHTFRHSYAMHMLYAGIPL |
| | KVLQSLMGHKSISSTEVYTKVFALDVAARHRVQFLMPESDAVTMLKNRQA |
| 1538 | WP_060594881.1 |
| | MLSSGITVRAAGDGFLDSIRSPNTRRSYAIAVDKTTARLGEARPLANVADDEIGETLETLWGEAAVNTWN |
| | ARRAAVASWLAWCREHGHLAPAVPAWVKRSTPPDSATPVRSRTAIDRLITRRDIDLRDKTLWRMLYETCA |
| | RTEELLQVNIEDLDLAGRCCPVKSKGAKPRTRRRGAAHHEYAHELVYWDAGTARLLPRLTKGRTRGPLFV |
| | THRRPGPRKAVADRDICPHTGLARLSYGQARALLDAATATNGPGTGWDLHELRHSGLTHLGEAGASLLEL |
| | MAKSRHRKPDNLRRYFKPSPAAMRGITSLLGPDRGHR |
| 1539 | WP_061770812.1 |
| | MTGKRKNSDDNWMPPRVYRGRSAYEFKHHNGSVIRLCSLDCTQSVVWAEYEKYIEQQKDNDTFKKLVGRF |
| | LISAEFTDLAFETQKDYHKYARKLIPVFGDVQPNDIRPEHVQRYMDKRGLKSKTQANREKTFMSRVFGWG |
| | YERGYVKGNPCKGVRQYKEKSRERYITDAEYAAVFAAAPDMVRAVMELAYLCCARQRDVLALTRNQILED |
| | GIYICQGKTGAKQIKAWSDRLRAAVALADSVPISTGIASAYVIHQQNGNRYTRDGFNSKWRNAKLAAKAA |
| | NPAMNIDFTFHDLKAKGISDLEGSLVEKQAISGHKNSSQTAIYDRKVKIVPVVGNQKK |
| 1540 | WP_075938737.1 |
| | MAKHFYLRDNQRIQNNSLLSSGSKELFTLEKSHSTIDAYESDWSDFCDWCNYRGIYFFPATPETIVNYIH |
| | DLSAYAKANTIARRVSALSENFTAGGLIKDNPCLSPLVKAAMKGIRRKIGTYQQGKSPLLKEDLEAIVOM |
| | MDIKDLTQHLDKTVLVIGFMGAFRRSELSSIRYEDVHFVRQGIEIFIPRSKADQEGEGNIVALPNLTQKE |
| | LCPVTTLKSWLSRTKITSGPVFRSPTKTGKLRKNALSDOMVNRIVKRWAEKIGLDPADYGAHSLRHGFAT |
| | SAALAGIEERRIMQQTRHHSVEMVRHYINEADRFEHNPLRDMFSAK |
| 1541 | ETI84668.1 |
| | MAKHFYLRDNQRIQNNSLLSSGSKELFTLEKSHSTIDAYESDWSDFCDWCSYRGIHYFPATPETIVNYIH |
| | DLSAYAKANTIARRVSALSENFTAGGLIKDNPCLSPLVKAAMKGIRRKIGTYQQGKSPLLKEDLEAIVOM |
| | MDIKDLTQHLDKTVLVIGFMGAFRRSELSSIRYEDVHFVRQGIEIFIPRSKADQEGEGNIVALPNLTQKE |
| | LCPVTTLKSWLSRTKITSGPVFRSPTKTGKLRKNALSDQMVNRIVKRWAEKIGLDPADYGAHSLRHGFAT |
| | SAALAGIEERRIMQQTRHHSVEMVRHYINEADRFEHNPLRDMFSSK |
| 1542 | WP_099738455.1 |
| | MSNKHSTISVAGKSHTRKTVKNITNRITKNKIVKSGSVQEYMDASQAGATKRAYGSDLRHFLAHGGAMPC |
| | TPKRLAKYLAESANDGLAVATLERRVTAIHKAHVDQKHGSPAHSEIVRQVMQGIRRTLGTKQRQVKPLTK |
| | DDLLPALETIESVHMPVRAARDRAILLIGFASAMRRSELVGVCVEHLTFSPAGLEIELPVSKTDQEQHGR |
| | TVFIPRANGSHCPVTALMCWLKTAGIRTGHVFRSVNRYDGIATQGLTPQSVALIVKGAMAQAGADARIFS |
| | GHSLRAGYCTTAAEQGLPSWQIRMQTGHKSDVTLARYIRKSDWQKAQSLL |
| 1543 | WP_066013827.1 |
| | MPSETEKSTSAPSAEHEVSRGDDRGHKESTSIALPAHVAGSGALDRLVDTARAYARAAASDNTLRAYAKD |
| | WAHFTRWCRMKGTDPLPPSPDIIGLYLADLASGSGALPSASRPLSVSTIERRLSGLTWNCAQRGFSLDRK |
| | NRHIAAVLAGIRRKHARPPVQKAAILAEDIVAMVATLPYDLRGLRDRAILFLGYAGGLRRSEIVSLDVHK |
| | DDTPESGGWIEILDKGALLTLNAKTGWREVEIGRGSTDQTCPVHALEQWLHFAKIDFGPVFVSTSRDGKC |
| | AYETRLNDKHVARLIKRTVLHAGIRPDLPETERLALFSGHSLRAGLASSAEVDERHVQKHLGHASAEMTR |
| | RYQRRRDRFRVNLTKAAGL |
| 1544 | WP_006120890.1 |
| | MMTASLPSLPGEYFQHTSRLPVAIDYPAALALRQMAAQLDDYPKYLLAPEVSALLHYIPDLYRKTLVDTL |
| | WNSGARINEALALGRTDFLLQPPYPFVQLATLKQRTEKSARTAGRAPAGSQAHRLVPLSDVNYVSQLEMM |
| | VATLKIPLERRNKRTGRTEKARIWEVTDRTVRTWLAEAVDAAAADGVTFSVPVTPHTFRHSYAMHMLYNG |
| | IPLKVLQSLLGHRSISSTEIYTKVFALDVAARHRVQFHMPGADAVAMIKGC |
| 1545 | PQV52181.1 |
| | MPNRLMPIARTDRRQLTAAEFHQLANVPPEAEWFGNLDNPRTRRAYQVDLRDFMAFIGIARPDEFRTVTR |
| | AHVLAWRKHLEARQLSGATIRRKLAALSSLFDYLCERNAVSLNPVAGVKRPKNNGNEGKTPALGDHQARA |
| | LLDAPDPVTLKGKRDRAMLAVLLYHGLRREELCLLKVRDIHDRRGTPHLRIHGKGSKLRYVPLHPASAER |
| | LHTYLESAGHDTVPDAPLFQPIRKTGTAITADGVYKCVLAWAVHAKIAVEGFGVHSLRATAATNALDHEA |
| | DIAKVQEWLGHANIATTRLYDRRKQRPEDSPTFKVAY |
| 1546 | WP_105508122.1 |
| | MPIARTDRRQLTAAEFHQLANVPPEAEWFGNLDNPRTRRAYQVDLRDFMAFIGIARPDEFRTVTRAHVLA |
| | WRKHLEARQLSGATIRRKLAALSSLFDYLCERNAVSLNPVAGVKRPKNNGNEGKTPALGDHQARALLDAP |
| | DPVTLKGKRDRAMLAVLLYHGLRREELCLLKVRDIHDRRGTPHLRIHGKGSKLRYVPLHPASAERLHTYL |
| | ESAGHDTVPDAPLFQPIRKTGTAITADGVYKCVLAWAVHAKIAVEGFGVHSLRATAATNALDHEADIAKV |
| | QEWLGHANIATTRLYDRRKQRPEDSPTFKVAY |
| 1547 | EJT85494.1 |
| | MSDAERYQQAARRASTARRYAQAIEHFEGEWGGLLPASSASVVRYLAAFGPQLSASTLRTHLAALAQWHQ |
| | RRGFVDPTKAAQVRDTLRGIQALHPQPVKQAPALQLKVLEASIEGLSADLHSALPVLRLRAARDQALILL |
| | GFWRAFRGDELCRLQAEHVRIEAGEGMQLFLPSSKTDRDNRGRNLTMPALKRLCPVQATEQWLLLSGIEQ |
| | GPLFRGIDRWGHINAQPLNANSVSRLLRRALLRSGIEAEGYSAHSLRRGFATWASRNQWSTEALMAYVGW |
| | RDVQSAARYIESHAPFGEWAR |
| 1548 | WP_035412914.1 |
| | MEEEMRNEVEIREAAALAFADSRERLLAVLEDDHLNMEALSDLEIFELFWATELFPIYSQKSPHTKRAYK |
| | QDLDYILRFFVTKTQGVKQLTILNLHEYLKDVHDQYAPRTVKRRNAMLRRFLRFLHVNDYHARDLSLQVK |
| | DQMKPEPLRREIDFDEMEAIALAFRHTVKQKKNRELLQLRNETMGYLLLTTGMRASELLSLQFNQIYQSK |
| | EFNYIEIKGKREKWRRIPLSEKTYYLLKYLNEKLISENILNPYVCFNINSTFSSITYETLRLITHEAAKV |
| | MGSEGNTPHWFRRSFITKMLTNNSPLIEVMKLSGHESITTTNKYLQDLKNERTINLPYN |
| 1549 | WP_005331670.1 |
| | MNYVKIYTKTYRDNSARYIELPSLLIEQDGETKVFEQLLKYQIKYSHKSKTWHNKLIQSVSLLFDYMNAN |
| | PNNYVSAKDFFELFAEAIYSGTIDEEGNDPSGLYWLPKKAQTANTLLSSLSDFSDWLYINYKTEQLNPWR |
| | EATRYEERLKYMALINRSERSFLGHLDDIHDISETAKTVRNVVTHKNPYAVRNGTKAFPEDKIEKLLREG |
| | FKKTRKGYELDLIDGYNWRDIAITILMHYGGLRHSEPFHLWVQDVIPDPEDPDMAIVRIYHPSEGKAPHD |
| | FKNPSTGKYVTDRASYLKLKYGLIPRNQYASSNKRFAGWKNPRLDDEDNMYMNVYWFPREAGYVFMYVWK |
| | KYLQQRIRYGIKDTHPFAFVSFDPRYLGEMMPPRTQTEAHNKACESIGLEISKFNGTTNHGHRHAYGQRM |
| | KNAGIDKKVRQVAMHHKSEESQNVYTEPTVTEVTNALSLATYSLNKGIALPMKSEISSWYEEEKKLAKKY |
| | MMRKK |
| 1550 | WP_010736891.1 |
| | MAQIKPYKKQDGTINYMFDFYVGINPKTGKKQKTTRRGFKTEEEASLALAELSLLVATEQYVPKKHHTFN |
| | EIFTLWYKQYCNLVKESTAVTAKCEFKYAILPKFQEMRIQDITPIYCQQIVNEWYKDKPKRASRFIYYFN |
| | RVMEHAMFLELIYQNPMANVKKTVTNLNLNHYEEFSNFFTKEELIEFLRFTKENFDSERYAFFRTLAFTG |
| | LRKGEAFALTWEDVNFDEKYLEVTKTVAKGDRNKILIHPPKTKAGFRRISLDNATLESLKTWREKQAIKF |
| | GLPKINPNQIIFSNYKNTYLESGITKEWFLTIQRLYRKKTGKEIKTITMHGFRHTHASLLYKANIPIKEA |
| | QERLGHSNVKTTLNIYTHLSKDQRDKTANRFAEFMNEI |
| 1551 | WP_010752316.1 |
| | MAKFEQYKKKNGEKAWKFQAYLGINPETGKPVKTTRRNFKTQREAKLALARLQSEYEDNLLKNDKPKTYK |
| | DVYDLWMTEYKRTVRGSTLLKTERIFKNHVLEELGDIYISEITPIKIQKLMDKWANKYDTAPKMMNYTGL |
| | VFKYAVRFGIIESNPTDAIRKPKRRKKATVEEPFYDKKQLKLFLDELYNQPNLKIQAFFRLLAMTGMRKQ |
| | EAGALEWRDIDFKAKTVNIYKAVTRTANGLEIDTTKTVGSSRIISIDQGTLDKLLEWKEAVLPPSDEWLV |
| | FGHSSAKNPHDIMSLDTSRKWLLNIQDQMDKKQKKKLPRITVHGFRHTQASLLIEMGASLKEVQFRLGHE |
| | DIQTTMNTYAHVSKLAKEQLADKFNKFIDL |
| 1552 | PKP94160.1 |
| | MVDRRTVDLVVQDVRGLVGPAVLLDTELVAAAVRGWSHNTRRAFRSDLCLWGDWCRRQRVAPASADAGVV |
| | AAWIRALAGMDPSGETVRAMATIERYVVNVGWAYRMAGLDDPTAAPLVRLEKKAARKHLGVRQRQARAIR |
| | FKGDIADFDSPASGVCLAHLLKACRRDLLGFRDEALLRTAYDSAGRRSELVAIDVDHIEGPDGQGAGTLF |
| | IPTSKTDRQGEGAYAYLAPSTMTAIARWREAGHIDRGALFRRVETHFDGSVAGVGRAALHPNSITLIYKR |
| | LIRAAHAKKLLGAMGEAELERWVSAVSSHSIRVGVAQDNFAADESLPAIMQAYRWRDPRTVMRYGAKLAP |
| | KSGASARMAKRFSES |
| 1553 | WP_014953267.1 |
| | MSNLTTDLKAIQEETLLNLKASKSNNTIRAYKSDFHDFGLFCVKNGFKSLPSNPKTVSLYLTYLATKNMK |
| | ISTIKRRLVSIAVVHKMKGHYLDNKHPSIIENLLGIKRRKGIKQKGKKPLLINNLKQIINVIDENNSSEI |
| | KIYRDRSIILLGFGGGFRRNELVSLNFDDLDFVNEGLKVSIRKSKTDQYGEGSIKALPYFDNPQYCPVKS |
| | LQKWLEISKIKEEAIFRKFHKGTKISNIRLSDQSVALLIKYYLNKAGIDSSDYSGHSLRSGFATSAAEAG |
| | AEERSIMEMTGHKSTEMVRRYIKEANLFKNNALNKIKL |
| 1554 | WP_065997227.1 |
| | MRGIRTISNSADVLRRAEALDALDAVLPFDRREFLAEILSDDDVETLRHLAREGIGENSMRALASDLAYL |
| | EAWCRAATSDPLPWPAPEALLLKFLAHHLWDPARRETDPTHGMPADVSAALMQAGLLRAKGPHSPSTVRR |
| | RLSSWSTLTQWRGLQGKFNAPRLRNATKLAVRASLRPRHRKSAKAVTADVLGALLKACAGDRLVDVRDRA |
| | LLMVAFASGGRRRSEVSSLRIAQLMEQDPVPADPDNPHSALLPCVSIHLGRTKTTEADDSAFVLLIGRPV |
| | AALDDWLARAGITEGAVFRRIDRWGHLERRALTPQAVNLILKRRILQCGLDPQEFSAHGLRAGFLTEAAR |
| | RGIPLPEAMQQSQHRSVQQASRYYNDAERRHGKAARLIV |
| 1555 | WP_015241550.1 |
| | MAKTPPSPSEDVHRRAEELDALDAILPFDRRDQLAALLTDDDVETLKHLASEGMGENTLRALASDLGYLE |
| | AWCRLATGAPLPWPAPEALLLKFVAHHLWDPVKRAEDPAHGMPADVEAGLRAERLLRSPGPHAPGTVQRR |
| | LTSWSILTRWRGLTGAFAAPSLKSTLRLAVRASARPRQRKSKKAVTVDILAKLLQACAGDRLVDLRDHAL |
| | LLTAFASGGRRRSEVAALRVEDLTDEEPVRADPSDKNSPPLPCLSIRLGRTKTTTADENEHVLLIGRPVA |
| | ALKTWLAEAQIKDGPVFRRIDQWGNIDRRALTPQSVNLILKARCEQAGLDPALFSAHGLRSGYLTEAANR |
| | GIPLPEAMQQSLHKSVTQAASYYNNAERKNGRAARLIV |
| 1556 | WP_113480034.1 |
| | MAKTTPSDAIHRRAEELDALDSILPFDRRDQLASLLTDDDVVTLKHLAGEGMGDNTLRALASDLGYLEAW |
| | CQLAIGGPLPWPAPESLLLKFVAHHLWDPVKRAEDADHGMPADVEAGLRDSRLLRAKGPHAPDTVRRRLT |
| | SWSVLTRWRGLTGAFNGPSLKSALRLAVRASARPRQRKSKKAVTADILAKLLQACAGERLVDLRDRALLL |
| | TAFASGGRRRSEIAGLRVADLVDEEAVRADPNDANSPRLPCLSIRLGRTKTTTSDDDEHVVLIGRPVVAL |
| | KHWFEQANVKDGPVFRRIDQWGNIDRRALTPQSVNLILKTRCKQAGLDPALFSAHGLRSGYLTEAANRGI |
| | PLPEAMQQSLHKSVTQAARYYNDSERKQGRAARLMI |
| 1557 | WP_104840046.1 |
| | MIKQHRTADSNSQALHRRAEELDALDAILPFDRRDQLAALLTDDDVATLKHLASEGMGGNTLKALASDLG |
| | YLEAWCRLATGSPLPWPAPEALLLKFVAHHLWDPVKRAEEPAHGMPADVEAGLRCEGLLRAKGPHAPGTV |
| | RRRLTSWSILTRWRGLSGAFGAPSLKSALRLAVKASSRPRQRKSKNAVTGDVLAKLLATCAGDRLVNRRD |
| | MALLLTAFASGGRRRSEVAGLRVEDLNDDEPVHADPADKTSPPLPCLSIRLGRTKTTTSDDDEHVLLIGR |
| | PVAALKRWLEDAGIKDGPVFRRIDQWGNVDRRALTPQSINLILKTRCKQAGLDPVLFSAHGLRSGYLTEA |
| | ANRGIPLPEAMQQSLHKSVTQAASYYNNAERKNGRAARLI1 |
| 1558 | PZN95492.1 |
| | MPGSPASPPKIGDLAVARINGGQPDIAEPDMETGAAAPATLISARLEALVETATGYAKAASSENTRAAYA |
| | KDWRHFSSWCRREGLEPLPPSSQVIGLYISACAAGEPKRGLPSLSVATIERRLSGLGWNFNQRGQPMDRA |
| | DRHISTVLAGIRRKHAKPPRQKEAVLGDDLLAMIATLGHDLRGLRDRAILLLGFAGGLRRSEIVGLDVVR |
| | DENSDGAGWIEIYADKGVLVTLRGKTGWREVEVGRGSSDHTCPVVALETWVRFGRIARGPLFRRIFKDNK |
| | TVDVERLSDKHVARLVKQTALEAGVRSDLPEGERALLFAGHSLRSGLASSAEIEERYVQKHLGHASAEMT |
| | RKYQRRRDRFRTNLTKASGL |
| 1559 | WP_057795742.1 |
| | MDSETEKSTSAPSGAVDDARGDESEQEAPNSIALPAHVAGSGTLDRLVDTARDYARAAASENTLKAYAKD |
| | WAHFARWCRMKGAEPLPPSPEMIGLYLADLASGSGPSPALAVSTIDRRLSGLAWNYAQRGFTLDRENRHI |
| | ATVLAGIKRKHARPPVQKAAILAEDILAMVATLPFDLRGLRDRAILLIGYAGGLRRSEIVSLDVGKDDTP |
| | DSGGWVEILEKGALLTLNAKTGWREVEIGRGSKKQSCPVHALEQWLHFARIDFGPVFVGTSRDGKRASET |
| | RLNDKHVARLIKRTALGAGIRADLPEKDRLALFSGHSLRAGLASSAEVDERYVQKQLGHASAEMTRRYQR |
| | RRDRFRVNLTKAAGL |
| 1560 | WP_089423562.1 |
| | MPSETEKSTSAPSDTNKDAPLDERDQKESDSIALPTHVAGSGTLDRLVYTARDYARAAASENTLKAYAKD |
| | WAHFARWLRMKGADPLPPSPEMIGLYLADLASGSGPSASQSASRPLSVSTIERRLSGLAWNYTQRGFTLD |
| | RNNRHVATVLAGIKRKHARPPVQKEAILAEDILVMVATLPYDLRGLRDRAILLLGYAGGLRRSEIVSLDV |
| | HKDDTPDSGGWIEIFDKGALLTLNAKTGWREVEIGRGSKDQTCPVHALERWLHFAKIDFGPVFVGSSRDG |
| | KRPSDTRLNDKHVARLIKRTVLNAGIRSELPEKERLALFSGHSLRAGLASSAEVDERYVQKHLGHASAEM |
| | TRRYQRRRDRFRVNLTKAAGL |
| 1561 | WP_023721997.1 |
| | MPDIVDVVDIISTEMGRGEASDEAPFHALRPAPGLPAHLERLADRARDYVDAASSANTRRAYASDWKHFC |
| | AWARRQHLEVLPPDPQTVGLYITACASGKVTGDKKPNSVATIERRLSSLAWNYTQRGEPLDRKDRHIATV |
| | LAGIRKSHAKPSVQKEAILPEDLIAMLQTLDRGTLRGLRDRAMLLLGFAGGLRRSEIVGLDVGRDQTDDG |
| | RGWVEILDKGALVTLRGKTGWREVEIGRGSADATCPVVALQTWLKLARIAHGPLFRRVTGQGKAVGAERL |
| | NDQEVARLVKRAALATGVRGDLSEGERQQKFAGHSLRAGLASSAEVDERYVQKQLGHASAEMTRKYQRRR |
| | DRFRVNLTKASGL |
| 1562 | WP_066052221.1 |
| | MERAVNEFASYLRNDKHSSENTVLSYIRDLRGFTEFMRVCGVSDALMVNYTNVMSYIYELQSKKKAGATV |
| | SRNIASIRAFYNYLIRQGAITDNPAANLELPKIEKKMPGILTLDKVEQLLEQPQGVDPKGIRDKAMLELL |
| | YATGIRVSELISLKVSDVNLPLEYIRCGVERKSRIIPIGSQAKAALRKYIEKGRSRMILADDEEMLFVNC |
| | NGKPMTRQGFWKIIKCYAKKAGIDEEITPHMLRHSFAAHLIENGADLKSVQEMLGHSDISSTQIYVKLTN |
| | QKLKSVYAKAHPRA |
| 1563 | WP_047138903.1 |
| | MRRTVLTYDVRVYSIETRKDRPKPYRLHWLVGDRKHSKSYTLRAQADGRRSELMTAARKGEQFDRDTGLP |
| | VSELRAQRGSVTWYQHTRAYIDRKWAAAPAKSRKNYADALATITPALVKAAKGRPDAALLRAALYGWAYN |
| | RNRWDLTPPEEIAAALAWVQKNSLPVSELEEAKTVRAALDALSLKLDGTPAAPRTARRKRACLSEVLGLA |
| | VEEKYFTVPVNPITTVKWTPPKSVEEVDPDSVANPRQVRALLRAVREQGPRGAHLEAFFGCLYYASMRPA |
| | EATALTLAQCHLPASGWGTLTLRKGAVRAGRGWTNDGSAHEARHLKARAEKDSRPVPIPQHFVRQLRQHV |
| | AVHGTAPDGRLFRTNRGGLLQETGYGEVWAAARQSALTEPEAASLLARRPYDLRHAGVSFWLSSGVDPME |
| | CARRAGHSVAVLLRVYAKVLARTQERANKRIEEAMRAWNEPE |
| 1564 | WP_005824123.1 |
| | MKKSTLSQQLFSQYFSDWVATYKEGAIRLVTMKKYRSTLHWIETLAPKLRVGDLSRITYQKLLNDYAQTH |
| | ERQTTMDFHHQLKGAILDAVDEGLLDTDPTRKAIIKGKAPKSKKIKYLNQFELQALLNSLELEQKINWDW |
| | FILLVAKTGMRFSEALAITPEDFDFAHQTLQVNKTWNYKEKGGFSPTKNRSSIRKIQLDWQTVIQFSQLI |
| | KDLPPDKPIFPCDTAIYNSTINGMLARICRKAKVSTISIHGLRHTHASLLLFAGVSIASVARRLGHSSMT |
| | TTQHTYLHIIQELENQDTDIVMRHLAGLC |
| 1565 | WP_000817856.1 |
| | MKREILLERIDKLKQIMPWYVLEYYQSKLAVPYSFTTLYEYLKEYDRFFSWVLESGISNADKMSDIPLSV |
| | LENMSKKDMEAFILYLRERPLLNANTTKQGVSQTTINRTLSALSSLYKYLTEEVENDLGEPYFYRNVMKK |
| | VSTKKKKETLAARAENIKQKLFLGDETEGFLTYIDQEYPQQLSNRALSSFNKNKERDLAIIALLLASGVR |
| | LSEAVNLDLRDLNLKMMVIDVTRKGGKRDSVNVAAFAKPYLENYLAIRNQRYKTEKTDTALFLTLYRGVP |
| | NRIDASSVEKMVAKYSEDFKVRVTPHKLRHTLATRLYDATKSQVLVSHQLGHASTQVTDLYTHIVNDEQK |
| | NALDSL |
| 1566 | WP_015217782.1 |
| | MLINRNGQAKVLTKSEIQQLFHNGFKSSRDKALFAVAFYTACRISEARKMFIIDAFYDGKVRDEIIIRKA |
| | HSKGKQGTRSIPTHPNLKKILQEYYDNSAKLVEMKKMIGDWSEKSFNCEGKIIINLAHQCPRCQFAGIFK |
| | NGVCNNQQRYKCKKCRHEFFERELPKTDLASENSSAIEFDPLGVTCSTLYGFLLEKSDNPFLFPGRRSKG |
| | YISLRNAMSIFVYAFDKLGIDGASTHSCRRTALTMMHREGVILKVLQEISGHKDLGALQKYLEVSEEQAR |
| | AAINIL |
| 1567 | WP_070726079.1 |
| | MSEDLSIVPASNATPTVSTQLARASAKVAGFLETGLQGAANTERAYTSDLKSYGAFCEHHGFVALPADVE |
| | TLTEYVAFLATEKPEPTLGDGREKKKGQQPLTRPHSLATIKRHLAAIRKAHQLAGHRLPATLDALNIVME |
| | GIARTLGKRQDQAQAFTVEELKQAILRIDLETSAGLRDRALLLLGFSGAFRRSELVDLNIEQLEFTERAL |
| | LVHLAKSKTNQYGAVEDKAIFYAPNADFCPVRCLRTWLNLLGWTTGPLFVKIPRAAPGQMAAPSDKRLSD |
| | ISINKLVQKRLGPAYSAHSLRVSFVTVAVLNGQSHKAIKNQTKQKTDAMIERYTQLNNVVSYNAAQALGL |
| 1568 | WP_000059622.1 |
| | MSLTDAKIRTLKPSDKPFKVSDSHGLYLLVKPGGSRHWYLKYRISGKESRIALGAYPAISLSDARQQREG |
| | IRKMLALNINPVQQRAAERGSRTPEKVFKNVALAWHKSNRKWSQNTADRLLASLNNHIFPVIGNLPVSEL |
| | KPRHFIDLLKGIEEKGLLEVASRTRQHLSNIMRHAVHQELIDTNPAANLGGVTTPPVRRHYPALPLERLP |
| | ELLERIGAYHQGRELTRHAVLLMLHVFIRSSELRFARWSEIDFTNRVWTIPATREPIIGVRYSGRGAKMR |
| | MPHIVPLSEQSIAILKQIKDITGNNELIFPGDHNPYKPMCENTVNKALRVMGYDTKKDICGHGFRAMACS |
| | ALMESGLWAKDAVERQMSHQERNTVRMAYIHKAEHLEARKAMMQWWSDYLEACRESYAPPYTIGKNKFIP |
| 1569 | WP_015369806.1 |
| | MAISKGAKRTDGLESADQVKLVVEEIAKKSQTVADLFLLGVETQLRGVDMRSWRWVELDIGSKVLRITOD |
| | KTKEAVEVELTETAREVLKARYNERGENVYVFQNDSNRSKGKPISRSKIHAEIQYAVDKLKMRGLLPQDA |
| | VISMHSSRKTIASIAHAQGEDLEVISKMLGHRSTEHTRAYLGITQAKVDALRTKYSTGIKRVTLR |
| 1570 | WP_013058885.1 |
| | MEFVKDVLNSFLEYLQIEKNYSKYTVDCYEKDIGIFMSFMQEEQIQNLOSVTYADARLFLTRLYEKQYSK |
| | RSMSRKISCLRTFYRYLNREELVEDNPFALVTLPKKEERNPRFLYEEEIVKLFQMNDLTTPLGQRNQSLL |
| | ELLYATGIRVSECASIKLSDIDFSLQTLLVYGKGKKQRYVPFGCYAKGALRVYIDNGRKLLLKKAPSDTH |
| | SLFLNYKGTPLTDRGIRLVIDQLVKKTAENIHISPHVLRHTFATHMLNEGADLRTVQEMLGHEHLSTTQI |
| | YTHVTKDRLKAVYMNHHPRA |
| 1571 | WP_013058263.1 |
| | MKKNSLEVIPLIDDFSQWLIESGKSDNTIKTYRAVLNQFHEWLLSEGRHLDQVTKNNVQTYMINLESNNK |
| | SASTIEKAFVTISVFARFLEKPEIVQNIERKRKEKNNEVVPQSLEASELDRLLSEVKQQGNLRDIAIVYT |
| | LLHTGVRVSEICALNHKDVEINKSDGFLIIRNAKGCKKRFVPLSTEARNSLKKYIDSLDSNHEALFVSNE |
| | DRRMSPRTVQYMLKKYNVNPHKLRHTFCHELVKKGIDIATVAELAGHSDVNVTKRYLKSSTRDLENAITQ |
| | TFL |
| 1572 | WP_056922110.1 |
| | MLPRIGGLRLRELTTPVVDRFVLDVYQDVGAATARTCRSIVSGALSLAVRQGAIAANPARELERLEGTRA |
| | KEPRALTSEEQAKWFMGMTGDQVAVRQDLVDFSAFLLATGLRIGEALAVLWTEVDLDTGALTVTSTLIRV |
| | TGQGLLRKTTKSKAGQRALLLPTWCVAMLRRRSEVGVAPDEPIFATVDGRFRDPRNVSRQLADARDRLGF |
| | GWVTSHTWRKTMATILDGGGASPRMIADQLGHSRVSMSLDFYLGRRSVDPRVLAALEAVDPRRFTLESGG |
| | QSGGSVAQGEGT |
| 1573 | WP_054448037.1 |
| | MTRYPKRGKGARWTVKELEAVPAEWAGDHLADGDGLTGEIRIQRGTMAVVWRYAYRLGDKVKRFYCGSWP |
| | ERTLDEIRTARNKARADIKAGRNPSAVRDLEKAQAREAVAAESAAVTAAEEAATTDALSVREMYKSWLES |
| | GVKRADGNTEVMRIVEKDVLPLIGDTAVRSIREKDIERVIRSIVGRGCNRLAEVTFQILGQMFHWAEKRQ |
| | PWRKLLSEGNPVELVELGVLLADDYDPDNVRERVLPPIEIVELQTRYRELEEQYLTSDDKRKRKPPSEAL |
| | QAVSWICLSTLCRIGELHLTEIAHLDLREGTWFIPKANVKGRKSQKRDHLVFLSPFAIKHFETLVSLAGS |
| | SRWLLPSRDNDAEVDQPMYKQAFTKQIKDRQAMFNGKSKARRASDNSLVLGKGQSGNWTPHDLRRTGSTI |
| | MESLGIDPNIIDRCQNHAIHTGKNRVRRHYQLYDYADEKQAAWAKLGEYLERLLSGAMAPAELQKRLTTK |
| | QLLAA |
| 1574 | WP_010744610.1 |
| | MDEQITEYLHYLSIERGLSDNTRISYQRDLHQYLSFLNDQGVTDWQAVDRYTVVAFLTSLTEAGKASTTI |
| | TRMISSLRRFHQFLRQERYTDHDPMQHIDSPKKAQKLPQTLSLTEVERLIAAPDTTTDLGIRDRAILEVM |
| | YATGLRVSELIGLRLGDIHLEMGLLQTIGKGDKERIVPLGDYAIHWLERYLSEVRPLLTKKTPNEMFLFV |
| | NNHGHGMSRQGIWKNLKQYVIKAEITKDVTPHTLRHSFATHLLENGADLRTVQELLGHADISTTQIYTHI |
| | TKRRMTEVYKEFFPRA |
| 1575 | WP_016179937.1 |
| | MDEQITEYLHFLTIERGLSENTRVSYQRDLHQYLSFLSEQGVTEWQAVDRYIVVAFLANLTEAGKASTTI |
| | TRMISSLRRFHQFLRQERYTDHDPMQHIDSPKKAQKLPQTLSLAEVERLIAAPDTTTDLGIRDRAILEVM |
| | YATGLRVSELIGLKLGDIHLEMGLLQTVGKGDKERIVPLGDYAIHWLERYLTEVRPLLTKKTPNVMFLFV |
| | NNHGHGMSRQGIWKNLKQYVIKAEIMKDVTPHTLRHSFATHLLENGADLRTVQELLGHADISTTQIYTHI |
| | TKRRMTEVYKEFFPRA |
| 1576 | WP_049220444.1 |
| | MDEQITEYLHFLTIERGLSENTRVSYQRDLYQYLSFLSEQGVTEWQAVDRYIVVAFLANLTEAGKASTTI |
| | TRMISSLRRFHQFLRQERYTDHDPMQHIDSPKKAQKLPQTLSLAEVGRLIAAPDTTTDLGIRDRAILEVM |
| | YATGLRVSELIGLKLGDIHLEMGLLQTVGKGDKERIVPLGDYAIHWLERYLTEVRPLLTKKTPNVMFLFV |
| | NNHGHGMSRQGIWKNLKQYVIKAEIMKDVTPHTLRHSFATHLLENGADLRTVQELLGHADISTTQIYTHI |
| | TKRRMTEVYKEFFPRA |
| 1577 | WP_088932358.1 |
| | MDEQITEYLHYLSIERGLSENTRISYQRDLQQYLSFLTDQGVSEWQAVDRYMVVSFLTNLTEAGKASTTI |
| | TRMISSLRRFHQFLRQERYTDHDPMQHIDSPKKAQKLPQTLSLAEVERLIATPDTTTDLGIRDRAILEVM |
| | YATGLRVSELIGLRLGDIHLEMGLLQTVGKGDKERIVPLGDYAIHWLERYLAEVRPILTKKTPNETFLFV |
| | NNHGHGLSRQGIWKNLKQYVIKAEIMKDVTPHTLRHSFATHLLENGADLRTVQELLGHADISTTQIYTHI |
| | TKRRMTEVYKEFFPRA |
| 1578 | WP_021268046.1 |
| | MAIRCYEKDGKKLYQVYVNARSKTDRKLRVQKTVSDLKSLSLARREENRINQELGKKLTELEGLCDTWES |
| | VIDKWEHEARSGFLGTYNPATIMDHVASLRNWTKSWLKTPASELGKANGRDLVKRMTNAEKSISFIKKVK |
| | NTVNLVYNFGIEEGLIKGVHQSPVYGIKLHHKKEKVPDILTLEEIKQFLYEARRQEHPWYPIWATALLTG |
| | MRSGELYALEWNDVDFENEIVRVSKSFNKRTNEIKSTKAGYWRNVPMSPELKELFISLKSSSKDKFVLPR |
| | FNDWRRGDQSKILKMFLIGNGLPKIKFHALRACFATQLLAKGTPAAIVMKICGWRDLKTMELYIRVAGVD |
| | EKGATDCLSILPSEVDVADNVVSLFHS |
| 1579 | WP_051517528.1 |
| | MTLIAQSTQAALDPIQLVLDSVTSPLTKAAYKKALTDFFVWWEEQGRPPLSKAVVQRHVALLVEQGLSPS |
| | SINVRLSALRKLVREAADNGLLGAFEAETIARVKGVKQQGRRSGTWLSKAQAQALLLAPDTTTLRGLRDR |
| | AILAVLLGCGLRRSELVGLTFAHLQQREGRWVILDLTGKHGRTRTVPMPAWCKAAVDAWTRTAGLSTGHV |
| | FRPTAPRGEHVLARQRLSHEAVALIVRKYGRQLGHNHLTPEDLEGVRLAPHDLRRTFAKLAHKGGAPIDQ |
| | IQLSLGHASIQTTEVYLGVDQDLESAPCDVLGLSLKGG |
| 1580 | WP_100251739.1 |
| | MTLPATLAARARAFADEALSENSRRAYRADWQHYARWCGGHDLAPLPAGPEQVASYLTSMAETHKRATIE |
| | RRLVTIGQAHKLQGLPWVPAHPAVRAALRGMFRRYGRPKKQAAALGVPETLRIVAACEGTVAALRDRALF |
| | LLSFAGAFRRSEVARIRHEDLAFRDGAVDVFLPHSKGDQDGEGTVVTVLAGGSPATCPIAALRRWLQAAP |
| | ADGYVFRAVRADGTVMDDGLHPDSVGRIVQKRAAEAGLVAGPRERISAHGFRAGFITEAYRRGSRDEEIM |
| | AHSRHRDLKTMRGYVRRAKLADAHPGRNLGL |
| 1581 | WP_020094536.1 |
| | MTLPATLAARARAFADEALSENSRRAYRADWQHYARWCSGHDLAPLPAGPEQVASYLTSMAETHKRATIE |
| | RRLVTIGQAHKLQGLPWVPAHPAVRAALRGMFRRYGRPKKQAAALGVPETLRIVAACEGTVAALRDRALF |
| | LLSFAGAFRRSEVARIRHEDLAFRDGAVDVFLPHSKGDQDGEGTVVTVLAGGSPATCPVAALRRWLQAAP |
| | ADGYVFRAVRADGTVMDDGLHPDSVGRIVQKRAAEAGLVAGPRERISAHGFRAGFITEAYRRGSRDEEIM |
| | AHSRHRDLKTMRGYVRRAKLADAHPGRNLGL |
| 1582 | WP_103985118.1 |
| | MTLPATLAARARAFADEALSENSRRAYRADWQHYARWCGGHDLAPLPAGPDQVASYLTSMAETHKRATIE |
| | RRLVTIGQAHKLQGLPWIPAHPAVRAALRGMFRRYGRPKKQAAALGVPETLRIVAACEGTVAALRDRALF |
| | LLSFAGAFRRSEVARIRHEDLAFRDGAVDVFLPHSKGDQDGEGTVVTVLAGGSPATCPIAALRRWLQAAP |
| | ADGYVFRAVRADGTVMDDGLHPDSVGRIVQKRAAEAGLVAGPRERISAHGFRAGFITEAYRRGSRDEEIM |
| | AHSRHRDLKTMRGYVRRAKLADAHPGRNLGL |
| 1583 | WP_014350944.1 |
| | MTEDTGALALPDPVRAQLRRGVRSVLVDTAALREVRQRFADDQAATLARYLEASQSANTVRAYRTDWIAW |
| | TAWCAAEGRQALPADALDVAVYLAAAADARTDDGAPAFAPATLERKSAAIAAVHAANGLPSPTRSDVVRL |
| | TLRGIRRTRRARPVRKRPILLHTLEQLLDGLPAPGWPTEPARRRDTLALLIGFAGALRRSELAALRVGDV |
| | HVTQDHTTGEPVLLIHLPTSKTDPTGITEQRVALPRGTRPHTCPVCAFADWIALLAVYTSAPGRLREQLT |
| | AAPQPDPNIHRCHGFTGLPPALLPDQPLFPAVTRHGGIGSTPISGRAIAELVKRYAARAGLDPALFSGHS |
| | LRAGFATQAALGGAADREIMRQGRWSNPRTVHRYIRTANPLDDNAVTKLGL |
| 1584 | WP_024545567.1 |
| | METSLAQPSPFSVPTDNPDILSQLLENQKSPHTWRAYKKDIRDFFRFVADANEPTPILIEALLKLEQPQA |
| | LALVLRYKNHLRDVRCLKEATINRRLAALKALVRLANQLGQCRYTLDGIRGEKVIHYRDTTGVSQNIYRQ |
| | ILKMPDQSTTKGKRDYAILRLLWDNALRRNEVVQTNLGDLDLERRSLDILGKGKGNQKEQITLSRATVTA |
| | LESWLTVRPGPKEKNQPLFVALDRAHQGHRLTGTAIYQLVRSTARAAGVQKVLSPHRIRHAGITAALDAT |
| | NGDVRKVQKFSRHADLNTLMIYDDNRRDVQGEITDLLAGLI |
| 1585 | WP_022614960.1 |
| | MTNLKKSNPFKNRVVRRADGISKNANEKALKKRSALSEAPSFKHYRKMLDTIYLYNPVLSLLFEMQSLTG |
| | LRYSDASTLIRNDFYDEVTGNFKPHFEFTPLKTYSLALDRIKNKDKNNSSSDDIEAKARNEAILTIFTND |
| | RIREVIDEVEELNGHIDSQFLFASEHVFSGGNPISIQYANRLLKRLHVDHPDLGFKETGTHSWRKYFATS |
| | MVELNGANLVQVQALLGHRDVNTTAKYVSKKKSDLQELIMQMKTEAA |
| 1586 | WP_071974181.1 |
| | MTRNDEKLRPEPPNAATTDGHNADGAALTLPAHVAGSGTLDRLVDTARDYARAAASENTLKAYAKDWAHY |
| | TRWCRMKGTEPLPPAPEMIGLYLADLAAGSGPSPSQAAHRPLSVSTIERRLSGLAWNVAQRGFTLDRRNR |
| | HIATVLAGIRRRHARPPVQKEAILADDIRAMVATLPHDLRGLRDRAILLLGYAGGLRRSEIVSLDVHKDD |
| | TPDSGGWIEIFDKGALLTLDAKTGWREVEIGRGSRDQTCPVHALEQWLHFAKIDFGPVFTGTSRDGRRAL |
| | DTRLNDKHVARLIKRTVLDAGIRSDLPDQERLKLFSGHSLRAGLASSAEVDERYVQKQLGHASAEMTRRY |
| | QRRRDRFRVNLTKAAGL |
| 1587 | WP_009557265.1 |
| | MPKRRAERGTVQFNNCNGSLRLLWTYQGERYSLALGLRNTPYHQKLANDRALWLTREIQYGRFNLEKLDQ |
| | YREFLRGENVSLSELPTVKAPPLSQLWQQYLEVRNLGKSPSTIRQYNWVTRHIDRLPTKDTRQPQAILDA |
| | IAKLSPDVQKRLLTQFCACAKWAQKSGLLTDNPFLGAAAAVKLPQRGTVEDEIHPFSRAERDQIIQAFRN |
| | DLHYQHYANLVAFLFFTGARPSEVVPLQWGHVKANYILFEKSRVDTVTGYQTKQGLKTQNCRRFPVNEQL |
| | RAILVGMERSDDESLVFPSVKGTYIQWNNFTNRAWKSVLSKLPEIEYRNPYQMRHSFVSHCRSLNVPSIQ |
| | VAEWIGNSVEMVDRVYAQVTESHSVPLL |
| 1588 | WP_069855669.1 |
| | MSSSRSVPAPATWENGVAAAAPPVLTDAMTARITESMAASRAESTTRAYASAWRRFEGWCTANGHVALPA |
| | HPASVAAYLVDAADTFTPDGERAYAPATFSKWIAAISHVHGRSGHTSPTTHETVRATLSGIRRSYASAGD |
| | RPRKQRAPLLVSDIVTMVTVARDSVTAWASEVLERRDSALLLMGFAGAFRRSELVGLNCGDVVVHRLDGL |
| | HIRLRKSKTDQDGDGAIRALPFTNSHTSCPPCAALRWWELVAAHERGGRAALIRTLRNAPAFDGHVCRGA |
| | LPKISPHAPFFRAIAKNGNLSTTALSAAAVHGAVRRRAGAAGYDESLVAALGGHSLRAGFVTQAFRNGAD |
| | AHAIMRQTGHKTPAMLEVYARENAPLIGNAVTDIGL |
| 1589 | WP_085421389.1 |
| | MTRIVDQNPENYPQEHSAASDSTADSADVSAPGASAGLPSPLPDANAGLPAHLQDLSDRARSYVEAASSA |
| | NTRKAYASDWKHFAAWCRRQNLSPLPPDPQVVGLYITACASGTAERGMKQNSVSTIERRLAAIGWNCSQR |
| | GMPLDRRDRAIATVMAGIRNRHAAPPRQKEAILPEDLIAMLETLDRGTLRGLRDRAMLLIGFAGGLRRSE |
| | ITGLDLGRDQTDDGRGWIEIFEKGLLVMLRGKTGWREVEIGRGSSDATCPVAAVETWIRFAKLAKGPLFR |
| | RVTSGGKDVGPDRLNDQEVARLVKKTALAAGVRGDLSEGGRAEKFAGHSLRAGLASSAEVDERYVQKQLG |
| | HASAEMTRRYQRRRDRFRVNLTKAAGL |
| 1590 | WP_062446129.1 |
| | MFPETISAVLQGASDRLVLAARSPATLRAYRTDWVAFVAWCSAQNVTALPAQPETVSAWIASRLEQGRKA |
| | GTLARGVAAVSCAHELAGFEGFSRSRVVQDALRGMRRTLGTAPTRKAPATVDLLRRMLDVQPNTLIGLRN |
| | RALLALGFAGALRRSELATLEVGDLVPQEGGALLTLRRSKTDPDGAGQTIGILNGSTIRALDHLAAWCEA |
| | ARITSGRLFRSVDRHGRTGESLSDRSVARIIKTAAEAVGLDPERFSGHSLRAGFITSGAEAGADALLIAE |
| | TSRHQSLDVLRTYVRRASLLKAHAGQRFL |
| 1591 | WP_008726205.1 |
| | MTATPKLQPNHTLDLFEKYLVARNKSPNTICVYRYAVEQFYHLYPQLTPRNLQLYKVYLLEHYKPQTVNL |
| | RIRALNCFMEYRQTSITPITMIKIQQKTYLDKIISQADYEYLKRKLVENEEFTYYFIVRLITTTGVRVSE |
| | LITFQIEDIDRGHKDIYSKGNKMRRIYVPTQLGIEFKQWFQHIGRRSGHLFLNRFGSPLSPSGIRAQFKV |
| | FAARYHLDPEVMYPHSFRHRFAKNFIEKCGDITLLSDLLGHESIETTRIYLRRSSSEQYRIINKVVDW |
| 1592 | WP_054528982.1 |
| | MNADAPEPPAQPSPAAALPVPFPDPFVAEVVEDVRDLVGAGVRLDAELVSAAVRGWSDNTRRAFRSDLTV |
| | WGDWCRRHGVVPARATPSHVAAFIRALSGIDPSAEEIRAMATIERYVSYIGRAYRLAGLPDPTSGELITF |
| | EKKAARKKRGVRQRQARAIRFKGDIADFDSPASGVCLAHLLKAVRRDEMGLRDEALMRVAYDVAARRSEV |
| | VAIDVDHIHGPDAQGAGALFIPSSKTDQEGEGAWGYLSPATMKAIARWREAARIDKGPLFRRIETHFDGS |
| | IAAIGTKRLHPNSINLIYKRLVQRAFDKKLLGPMSEAEVARWVAAVSSHSLRVGVAQDNFAAREPLPAIM |
| | QAYRWRDPKTVLRYGAQLAVKSGAAARMAARVNES |
| 1593 | KPL69881.1 |
| | MPVPFPDPFVAEVVEDVRDLVGAGVRLDAELVSAAVRGWSDNTRRAFRSDLTVWGDWCRRHGVVPARATP |
| | SHVAAFIRALSGIDPSAEEIRAMATIERYVSYIGRAYRLAGLPDPTSGELITFEKKAARKKRGVRQRQAR |
| | AIRFKGDIADFDSPASGVCLAHLLKAVRRDEMGLRDEALMRVAYDVAARRSEVVAIDVDHIHGPDAQGAG |
| | ALFIPSSKTDQEGEGAWGYLSPATMKAIARWREAARIDKGPLFRRIETHFDGSIAAIGTKRLHPNSINLI |
| | YKRLVQRAFDKKLLGPMSEAEVARWVAAVSSHSLRVGVAQDNFAAREPLPAIMQAYRWRDPKTVLRYGAQ |
| | LAVKSGAAARMAARVNES |
| 1594 | SEM26217.1 |
| | MLSGMAENIEKSSSEAANVSSSNDDNERDRQDGEALSLPSSVAGSGALDRLVETARDYARAAASENTLKA |
| | YAKDWTHFARWCRMKGAEPLPPSPEMIGLYLADLASGSGPSSTLSVSTIDRRLSGLAWNYAQRGFTLDRK |
| | NRHIATVLAGIKRKHARPPAQKEAILAEDILAMVATLPYDLRGLRDRAILLIGYAGGLRRSEIVSLDVGK |
| | DNTPNSGGWIEILENGVILTLNAKTGWREVEIGRGSSEQTCPVHALEQWLHFAKIDFGPVFVRTSRDGKK |
| | ALEARLSDKHVARLIKRTVLDAGIRSDLPEKDRLALFSGHSLRAGLASSAEVDERYVQKQLGHASAEMTR |
| | RYQRRRDRFRVNLTKAAGL |
| 1595 | WP_106165551.1 |
| | MASETERSTSARSDELDDAPLDERDQRNSNYIALPSHVAASGALDRLVDTARNYARAAASDNTLKAYAKD |
| | WAHFARWCRMKGAEPLPPAPEMIGLYLADLASGSGPSPSRSASRSLSVSTIDRRLSGLGWNFAQRGFTLN |
| | RKNRHIATVLAGIKRKHARPPVQKAAILAEDILAMVATLPFDLRGLRDRAILLLGYAGGLRRSEIVSLDV |
| | HKDDTPDSSGWIEIMEKGALLTLNAKTGWREVEICRGSKDQTCPVHALEQWLRFAKIDFGPVFVGTSRDG |
| | KRALETRLNDKHVARLIKRTVLDAGIRSDLPDSERLALFSGHSLRAGLASSAEVDERYVQKQLGHASAEM |
| | TRRYQRSRDRFRVNLTKAAGL |
| 1596 | WP_008335838.1 |
| | MPSETEKSSSTPSDELNDARVDERAREESDDIALPSHVAGSGTLDRLVDTARDYARAAASDNTLKAYAKD |
| | WAHFTHWSRMKGAEPLPPSTEMVGLYLADLASGSGLSPALSVSTIDRRLSGLAWNYAQRGFTLDRKNRHI |
| | ATVLAGIKRKHARPPVQKEAILAEDILAMVATLPYDLRGLRDRAILLVGYAGGLRRSEIVSLDVHKDDTP |
| | GSGGWIEIFDKGALLTLNAKTGWREVEIGRGSKEQTCPVHALKQWLDFAKIDFGPVFVGTSRDGKRTSET |
| | RLNDKHVARLIKRTVLDAGIRSELPEQERMALFSGHSLRAGLASSAEVDERFVQKHLGHTSAEMTRRYQR |
| | RRDRFRVNLTKAAGL |
| 1597 | WP_029069676.1 |
| | MVNPMESHLTSTHTGPLAFPSEHDVLRLVDHSRSDNTHRTYDVGVRSWARFASTYSYQAFPADPAEVALW |
| | LSALFDEGKSTATAKTYLQSLRDHHRERGSSALNDIEGLRRVMQGIQRLNRERDARKARALSPTELMMLV |
| | GQSRMSGTLRGTRDTAWWLLCTSLGLRYSDAAILERRDIRFVEEKGAVVTLRFSKTDQFARGTDLALARA |
| | RFAHVDPVMALTDLLKALPEDPHTPVFQSVLKSNRWSGRSLTNTGLNKAIRRLADDTGINGERLTAHSAR |
| | VTFATNAYAAGIDESAIAITGRWKSLSVQRSYRRVDDESLFDKRSTASYWLEETLSR |
| 1598 | WP_011886969.1 |
| | MAGSIEKRGKNSYRLVYSMGFDANGKRIKRTKTVHVKTKKEAEKELAKFIAEIEAGEYIKPAKMSLSDFI |
| | QLWRDNYAEKQLSPKTFETYNNYINTRIIPQLGHLQLADIKPIHLIRFLNNLKKDETRLDGKKGSLSEAT |
| | INYYRRILKNIFNRAVEWKFLQVNPAEKLPKEKEDIGKGDVYDENETRLLLKCLEKEDLKWRLYFTLALT |
| | CGLRKGELLALQWEDIDLESGTLYVKHSLSYTKEKGFFLKEPKSKKSKREIAIPSFVLPLLKKYKNVRLR |
| | EKEKLQDEWEGGNYNFVFATWNGKPHHHSYPRTKWERFLKRNNLRYIRPHDLRHTSATIMLNNGVNYKTV |
| | SERLGHSSTRITFDFYVHRTKEADRSAAECFDNQFGA |
| 1599 | WP_047821448.1 |
| | MPLTDTRVRQLKHTGKPTGDKYTDSRSLHLLVKEAGKYWRMSYRFDGKQRTLALGVYPSVTLAKARQLRD |
| | QARQLLSEGVDPVEAKRRDKVAKESAAKHSFEAVARDLLKLRACSLAPSTIRKNTAWLEKNVFPEMGMMP |
| | ISKIEPRDVLFMLRKIEARGAIESTHKIRQLCGQVFRFAVASGLASRDVTFDLRDALPSVPEVHYAAITE |
| | PKQAAALMRSISNYSGHPYSRAALRLAPLFFVRPGVLRAAEWSEFDLDRGVWFIPATKMKIRQPHIVPLA |
| | RQAVGILRSMHQLTGHAKYVFPSIRAKDRCMSENTINAALRAMGYSKDMMTGHGFRAMARTILDEVLGER |
| | VDLIEHQLAHAVRDANGRAYNRTTHLPARIEMMQRWADYLDQIALPQATS |
| 1600 | WP_047825138.1 |
| | MPNFTPIDLGPSAPEEITSSPSGRAHVKMYRLADDAITEATTDIELAREFIAHGELSAKSIQNSQKELYR |
| | FLTWCREEARKTLVQLNVADLNAYKDFLKNPPPEWISRTKWPRSDPRYRPFTGPLSDPSRRQAMIAVKGL |
| | MGFAEQTGYLRRNPGALVRNVRAPSASRITRYLTQNAIALALQTVSARPADTPAAFRRRARDRFLLIAFA |
| | HTGARLNEIVSASMGSIYTEGNGRWWLDVLGKGNKPRRLPVPPDMLEAFQSYRQAFELLPQSSRTDRTPL |
| | VLSSRSRELARITDEAAAEAIKAVFADAARAADAQGDQDTAATLRQASAHWLRHSMLTNHANNGVQLKTL |
| | QDTAGHANIATTAAYLHKTDNERHDEIIRSANGNGIL |
| 1601 | WP_116546838.1 |
| | MALSQHQQALSQLQSFNALPLHLRSMATAHQQFTRYAEDSYSANTLRMVDFAEKHWAHWLAKQTDLPAEC |
| | WHEQQLLLYPIWPDILCRYIDELSESMSLNSVQTYINLLNFKNKKLGFPSLLQHTHVQWAMRRATNRALD |
| | AGEQIGQAQPFRLHDLELLLQIFADTDDPKLMRDLLLVWIAYESLLRESELVNIRCNDLLPGRQYSIRVR |
| | KTKTTKTLEDNEVLLSEPCSQWLHRYMTHFGLPLSSSGYLFRRLKKNGELFHSEENCKKLSGRTVDDIFR |
| | FFYWQIDPDARAELQNSIHAADASRYQTWTGHSARVGAAIDLFVYGASVHEIMRLGRWRNDQTVMRYIRR |
| | VSMQELPMNRMVTERLKR |
| 1602 | WP_086904734.1 |
| | MSKSIIHYSTGGSAPSRSSGIASNFTSSDKQIDTPFFEESSLPQSVHSDFFNAAAETEYE1SINTRRVYR |
| | TSFGLFEQYCATHQLQSLPADPRSIISFIGHQKELLQASSGTQLSKQTLTTRLAAIRYYHIQAGFPSPTE |
| | HPLVIRVMRGLSRNHHRQVQDYDQQPIMYDEVELLIQAIEQQPHPLLRSRDKAIIQLGLQGGFRRSELAN |
| | LKVQYLSFMRDKLKVRLPFSKSNQQGLREWKNLPDSEPFAAYNAVKDWLNESKITEGHLFRSISRDGKTL |
| | RPYQVSDNVTSKSSLIRNSGFLNGDDIYRIIKQYCLKAGLPAQYYGAHSLRSGCVTQLHENNKDTLYIMA |
| | RTGHTDPRSLRHYLKPKED |
| 1603 | WP_133181036.1 |
| | MSKSLNHYFAGDNTPTRISGMASTITPVYKQTDSPFFEESSLPQSVHSDFFNAAAETEYEISSNTRRVYR |
| | TSFGLFEQYCATHQLQSLPADPRSIISFIGHQKELLQASNGTQLSKQTLITRLAAVRYYHIQAGFPSPTE |
| | HPQVIRVMRGLSRNHHRQVQDYDQQPIMYDEVELLIQAIEQQPHPLLRTRDKAIIQLGLQGGFRRSELAN |
| | LKVQYLSFMRDKLKVRLPFSKSNQQGLREWKNLPESEPFAAYNAIKDWLHESKITEGHLFRSISRDGKSL |
| | RPYQVSDKVTSKSSLVRNSGFLNGDDIYRIIKQYCVKAGLPAQYYGAHSLRSGCVTQLHENNKDTFYIMA |
| | RTGHTDPRSLRHYLKPKED |
| 1604 | WP_109285990.1 |
| | MSKSIIHYSTGGSAPSRSSGIASNFTASDKKMDTPFFEESSLPQSVHSDFFNAAAETEYEISINTRRVYR |
| | TSFGLFEQYCVAHQLQSLPADPRSIISFIGHQKELLQASSGTQLSKQTLTTRLAAIRYYHIQAGFPSPTE |
| | HPLVIRVMRGLSRNHHRQVQDYDQQPIMYDEVELLIQAIEQQPHPLLRSRDKAIIQLGLQGGFRRSELAN |
| | LKVQYLSFMRDKLKVRLPFSKSNQQGLREWKNLPDSEPFAAYNAVKDWLKESQITDGHLFRSISRDGKTL |
| | RPYQISDKVTCKSSLVRNSGFLNGDDIYRIIKQYCVKAGLPSQYYGAHSLRSGCVTQLHENNKDTLYIMA |
| | RTGHTDPRSLRHYLKPKED |
| 1605 | WP_113940403.1 |
| | MSKMIRTNSNAQNNTNVTNERVTGSDHHNNNRAEQPRFFEETFLPQSVRSDYLSAAEETEYEISANTRRV |
| | YNTSFSLFSRYCAEHQLQALPADPRSVISFIGYQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALNAVKNWLSLANIEDGHLFRSLSRDGK |
| | YLRPYQIVEHHSEANSSLHKNSGFLTGDDIYRIIKKYCTKAGLPAKFYGAHSLRSGCVTQLHENDKDHLY |
| | IMARTGHTDPRSLRHYLKPRD |
| 1606 | ACK46586.1 |
| | MSKMIRTNSNAQNNTNISNERVIGSGHHHNNRAEQPRFFEESFLPQSVRSDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | KLRPYQMKNRHSGSNSLLNKNSGFLTGDDIYRIIKKYCTKAGLPAKFYGAHSLRSGCVTQLHENNKDHLY |
| | IMARTGHTDPRSLRHYLKPRD |
| 1607 | AEG11408.1 |
| | MSKMIRTNSNAQNNANISNEIATGSGHHHNNRAEQPRFFEETFLPQSVRSDYLSAAEETEYEISVNTRRV |
| | YNTSFNVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVHYVSFLRNKLKVRLPYSKSNQQGQREWKDLPDHEPFAALDAVKNWLSLANIEDGHLFRSLSRDGK |
| | NLRPYQMKDRHSGSSSLLNKNSGFLTGDDIYRIIKKYCTKAGLPAKFYGAHSLRSGCVTQLHENNKDHLY |
| | IMARTGHTDPRSLRHYLKPRD |
| 1608 | WP_081248413.1 |
| | MSRMIRTNINAQNNTNISNERVIGSGHHHNNRAEQPRFFEESFLPQSVRSDYLSAAEETEYEISVNTRRV |
| | YNTSFSVFSRYCAEHQLQALPADPRSVISFIGHQRELIQESTGVQLSRQTLTTRLAAIRYHHIQAGFHSP |
| | TEHPLVIRVMRGLSRNQSRHVSDYDQQPIMYDEVEMLIQAIDEQVQPLTRARDRAIIQLGLQGGFRRSEL |
| | ADIRVQYVSFLRNRLRVRLPYSRSNQQGQREWRDLPDHEPFAALDAVRNWLSLANIEDGHLFRSLSRDGR |
| | NLRPYQMRDRHSGSSSLLNRNSGFLTGDDIYRIIRRYCTRAGLPARFYGAHSLRSGCVTQLHENNRDHLY |
| | IMSRTGHTDPRSLRHYLRPRD |
| 1609 | WP_012277158.1 |
| | MNSEQQCPRQVPSLNEQEHALGHFSGGLTNGHSTQHAPSQNPNERFFQEQQLPISILDDYRSAASETQYE |
| | ISDNTRRVYRSSFAIFRNYCDQHNLSALPADPRSVISFIGHQREIYQERSGHQLSRQTINTRLAAIRFFH |
| | IQAAHHSPTEHPLVIRVMRGLMRNQYRQISDYDQQPITYDELEMLLDVIERQPQQLTRLRDRAILQLGLQ |
| | GGFRRSELAEIRVEHISFLRERLRVRVPYSRSNQQGQREWRDLPRQELFSAYEAVQQWLDATRIRQGHLF |
| | RSLSRDGNSVRDYQITQARMGRGFLRGDDIYQMIRRYCDRAGLNSRFYGAHSLRSGCVTQLHENDRDHLY |
| | IMARTGHTDPRSLRHYLRPRD |
| 1610 | WP_012586824.1 |
| | MASYSIQRRERADGTVRHRCLVRVRRNGRILYTEQRTFTRYAAAEAWGRDRVIDIESNGFATEDTAPITL |
| | GSIISRALTDENIDSSIGRSRRFCLRLLSDCDIARLNLTDIRPHHIIDHCRLRRSAGTGPSTIAVDVSVI |
| | RWLLRIARSNFGHEVSQISVIEAYDALYSQDLIARSGRRSRRPTTDEIERLRVGLAARADQRAAHIPYID |
| | LLDFSILSCMRIGEVCRITWDDVDEAQRAVIVRDRRDPRRRAGNHMLVPLLGGAWEILQRQPRNDARVFP |
| | YNERSVTAGFQRVRNELGIEDLRYHDLRREGASRLFERGYSIDEVAQVTGHRNINTLWQVYTELFPRRLH |
| | DRDC |
| 1611 | WP_081729030.1 |
| | MTGSDHHNNNRAEQPHFFEETFLPQSVRSDYLSAAEETEYEISANTRRVYNTSFSLFSRYCAEHQLQALP |
| | ADPRSVISFIGHQKELIQESTGVQLSKQTLTTRLAAIRYHHIQAGFHSPTEHPLVIRVMRGLSRNQSRHV |
| | SDYDQQPIMYDEVELLIQAIDEQVQPLTRARDKAIIQLGLQGGFRRSELADIKVQYVSFLRNKLKVRLPY |
| | SKSNQQGQREWKDLPDHEPFAALSAVKNWLSLANIEDGHLFRSLSRDGKYLRPYQIVEHHSEANSSLHKN |
| | SGFLTGDDIYRIIKKYCTKAGLPAKFYGAHSLRSGCVTQLHENDKDHLYIMARTGHTDPRSLRHYLKPRD |
| 1612 | KZK70296.1 |
| | MIGSGHHHNNRAEQPRFFEESFLPQSVRSDYLSAAEETEYEISVNTRRVYNTSFSVFSRYCAEHQLQALP |
| | ADPRSVISFIGHQKELIQESTGVQLSKQTLTTRLAAIRYHHIQAGFHSPTEHPLVIRVMRGLSRNQSRHV |
| | SDYDQQPIMYDEVEMLIQAIDEQVQPLTRARDKAIIQLGLQGGFRRSELADIKVQYVSFLRNKLKVRLPY |
| | SKSNQQGQREWKDLPDHEPFAALDAVKNWLSLANIEDGHLFRSLSRDGKNLRPYQMKDRHSGSSSLLNKN |
| | SGFLTGDDIYRIIKKYCTKAGLPAKFYGAHSLRSGCVTQLHENNKDHLYIMSRTGHTDPRSLRHYLKPKD |
| 1613 | WP_012154534.1 |
| | MANSTKQLTATQVSNAKPKEKEYNLADGRGLSLRVKTGGSKFWLLNYTRPVTQKRANLGLGTYPDVPLAE |
| | ARKRREAARELLAQGIDPQHHQQQQKAAIKTDAENTLKSVTNAWFEIKKQKVSENHGQKLYRRLELYLFP |
| | ALGGTPISVLTAPQVIQVLKPAEAKGNIETCKRVISWLNEVMTFAVNTGLIHSNPLIGIAAAFGVPEKRQ |
| | MPTLKPAELPEFIEALTYSSIKKTTRCLIEIQLHTMTRPAEAAKAKWTEIDFDKQLWTIPAERMKMKREH |
| | IIPLTPQVISLLNRMHEISGDLEYIFPADRNKHHHTNTETANMAIKRMGYKGRLVAHGLRALASTTLNEQ |
| | GFDAELIEVSLAHVDKNTVRAAYNRADYIERRRELMCWWSEHVQITPNQLNSVITQQLLK |
| 1614 | ABV87414.1 |
| | MLDLTSLLQIKAKDLKMNSEQNFPEIEGFSQIEDSDLIENAPQEVAIVDGESALTRFNSGLAESRTSQFD |
| | HNEKFFKEQQLPISILDDYKSAAGETQYEISANTRRVYRSSFTIFKNYCDQHNLSPLPADPRSVISFIGH |
| | QKELYQEKNGHQLSKQTINTRLAAIRFFHIQAALHSPTEHPLVIRVMRGLMRNQYRHVSDYDQQPITYDE |
| | LEMLLAVIDQQPKELTRLRDKAILQLGLQGGFRRSELAEVRIEHISFLREKLKVRVPYSKSNQQGQREWK |
| | DLPKQELFSAYDAVQQWLDATKIKQGHLFRSLSRDGNSVREYQITQEKIGKGFLKGDDIYQMIKKYCDKA |
| | GLNSRFYGAHSLRSGCVTQLHENDKDHLYIMARTGHTDPRSLRHYLKPKD |
| 1615 | WP_011622713.1 |
| | MSKSIIHYSTGGNAPSRSSGIASNFTSSDKOMDTPFFEESSLPOSVHSDFFNAAAETEYEISINTRRVYR |
| | TSFGLFEQYCTAHQLQSLPADPRSIISFIGHQKELLQASSGTQLSKQTLTTRLAAIRYYHIQAGFPSPTE |
| | HPLVIRVMRGLSRNHHRQVQDYDQQPIMYDEVELLLQAIEQQPHPLLRSRDKAIIQLGLQGGFRRSELAN |
| | LKVQYLSFMRDKLKVRLPFSKSNQQGLREWKNLPDSEPFAAYNAVKDWLNESKITEGHLFRSISRDGKTL |
| | RPYQVSDNVTSKSSLIRNSGFLNGDDIYRIIKQYCLKAGLPAQYYGAHSLRSGCVTQLHENNRDTLYIMA |
| | RTGHTDPRSLRHYLKPKED |
| 1616 | WP_051714141.1 |
| | MSKTNRFYPIDVNQQSVGVNTHLTKKLTQADNAFFEESALPQSVHNDFYNAAAETEFEISSNTRRVYQTS |
| | FSLFAQYCLEHRLQSLPTDPRSVISFIGHQKELLMADTGMQLSKQTLTTRLAAIRYYHIQAGFPSPTEHP |
| | LVLRVMRGLSRNHNRRVQDYDQQPIMYDDVELLLQAVEQQPHPLLRSRDKAIIQLGLQGGFRRSELANLK |
| | VQYLSFMRDKLKVRLPFSKSNQQGLREWKNLPDSEPFAAYHAVKAWLHESQISDGHLFRSISRDGKTLRP |
| | YQVKDNNKSNTTFNRNSGFLNGDDIYRIIKQYCVKAGLPAQYYGAHSLRSGCVTQLHENNKDTLYIMART |
| | GHTDPRSLRHYLKPKED |
| 1617 | WP_077751411.1 |
| | MNKLSINQNHRQQVTGDKSFFEEQELPISIFDDFKSAASETEYEVAPNTRRVYRSSFNIFTQYCQHHGLN |
| | NLPADPRSVISFIGHQKEQVHKKTGAQFSKQTITTRLAAIRFYHIQAGFHSPTEHPLVIRVMRGLSRNKH |
| | RVITDYDQQPIMYDELELLLQTIDKQGQELTKARDKAIIQLGFQGGFRRSELAEIQVKHINFLRNKLKVR |
| | LPYSKSNQQGHREWKDLPGSELFSAFGAVKHWLDVSQLSQGHLFRSLSRDGQSLRPYSVVNQANLNTDEN |
| | PPQLNRGFLRGDDIYQMIKKYCSKAGLSPEFYGAHSLRSGCVTQLHENDKDHLYIMARTGHTDPRSLRHY |
| | LKPKD |
| 1618 | WP_013051410.1 |
| | MNKLSINQFNRPAITSDKSFFQEQELPISILDDFKSAASETEYEVADNTRRVYRSSFNIFTEYCQHHGLN |
| | HLPADPRSVISFIGHQKEQVHHRTGMQFSKQTITTRLAAIRFYHIQAGFHSPSEHPLVIRVMRGLSRNKH |
| | RLTSDYDQQPIMYEELELLLQTIDKQEQELTRARDKAIIQLGFQGGFRRSELAEIQVNHVNFLRNKLKVR |
| | LAYSKSNQQGHKEWKDLPESEQFSAFSAVRHWLEVSQLTQGHLFRSLTRDGQRLRPYSVASRVNLNSHDN |
| | LPQVNRGFLRGDDIYQMIKKYCRKAGLSPEFYGAHSLRSGCVTQLHENDKDHLYIMARTGHTDPRSLRHY |
| | LKPKD |
| 1619 | WP_115334556.1 |
| | MSKMIRTNSNAQNNANISNERATGSDHHHNNRVEQPRFFEETFLPQSVRSDYLSAAEETEYEISVNTRRV |
| | YNTSFNVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | NLRPYQMKDRHCGSSSLLNKNSGFLTGDDIYRIIKKYCTKAGLPAKFYGAHSLRSGCVTQLHENNKDHLY |
| | IMARTGHTDPRSLRHYLKPKD |
| 1620 | WP_126491884.1 |
| | MSKMIRTNSNAQNNANISNERVKESDHHHNNRAEQPRFFEESFLPQSVRSDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQMQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | NLRPYQMKDRHSGSSSLLNKNSGFLTGDDIYRIIKKYCTKAGLPAKFYGAHSLRSGCVTQLHENNKDHLY |
| | IMARTGHTDPRSLRHYLKPKD |
| 1621 | WP_020912617.1 |
| | MSRKHISPISNKVSSTSSNNDFYQEAELPISMLNDFESAAKETRYEISNNTRRVYRSSFGIFKAYCDAHG |
| | RSSIPADPRTVISFIGHQKDFYQAKSGHQLSTQTINSRLAAIRFYHIQSGTPSPTEHPLVTRVMRGLMRN |
| | HTRIVSDYDQQPIMYEELEILIQAIENQSQPLTQKRDKAIILLGFQGGFRRSELANIKVNHLSFLRDKLK |
| | VRLPYSKSNQQGQREWKVLPKGETFSAYEPIKDWLNAAKIKEGHLFRSLTRDGRYIRDYQVLDANSGKGF |
| | LRGDDIYQLIKRYCNKADLDPKFYGAHSLRSGCVTQLHENNKDHLYIMGRTGHTDPRSLNHYLKPND |
| 1622 | WP_088211152.1 |
| | MSKSIIHYSTGGSAPSRSSGITSNITSSDKQMDPPFFEESSLPQSVHSDFFNAAAETEYEISINTRRVYR |
| | TSFGLFEQYCATHQLQSLPADPRSIISFIGHQKELLQASNGTQLSKQTLTTRLAAIRYYHIQAGFPSPTE |
| | HPLVIRVMRGLSRNHHRQVQDYDQQPIMYDEVELLIQAIEQQPHPLLRLRDKAIIQLGLQGGFRRSELAN |
| | LKVQYLSFMRDKLKVRLPFSKSNQQGLREWKNLPDSEPFAAYNAVKDWLHESKITEGHLFRSISRDGKTL |
| | RPYQVSDKVTSKSSLVRNSGFLNGDDIYRIIKQYCLKAGLPAQYYGAHSLRSGCVTQLHENNKDTLYIMA |
| | RTGHTDPRSLRHYLKPKED |
| 1623 | WP_011626197.1 |
| | MSKSIIHYSTGGSAPSRSSGIASNITASDKKMDTPFFEESSLPQSVHSDFFNAAAETEYEISINTRRVYR |
| | TSFGLFEQYCATHQLQSLPADPRSIISFIGHQKELLQASSGTQLSKQTLTTRLAAIRYYHIQAGFPSPTE |
| | HPLVIRVMRGLSRNHHRQVQDYDQQPIMYDEVELLIQAIEQQPHPLLRLRDKAIIQLGLQGGFRRSELAN |
| | LKVQYLSFMRDKLKVRLPFSKSNQQGLREWKNLPDSEPFAAYNAVKDWLKESQITDGHLFRSISRDGKTL |
| | RPYQISDNVTCKSSLVRNSGFLNGDDIYRIIKQYCVKAGLPSQYYGAHSLRSGCVTQLHENNKDTLYIMA |
| | RTGHTDPRSLRHYLKPKED |
| 1624 | WP_011072365.1 |
| | MSKSIQIYTADDSHSHQAVGISANLTKPFTQGDKTFFEESSLPQSVHADFYNAASETEYEISNNTRRVYR |
| | ISFSFFEQYCLEHNLQSLPADPRSIISFIGHQKELLQASTGMQLSKQTLTTRIAAIRFYHIQAGFPTPTE |
| | HPQVIRVMRGLSRNHHRLVQDYDQQPIMYDEVELLIQAVDQQPHPLLRLRDKAIIQLGLQGGFRRSELAN |
| | LKVHYLSFMRDKLKVRLPFSKSNQQGLREWKSLPDSEPFAAYHAVKSWLNESQITDGHLFRSISRDGKTL |
| | RPYHVNDNSKPKSTFSRNSGFLNGDDIYRIIKQYCLKAGLPAQYYGAHSLRSGCVTQLHENNKDILYIMA |
| | RTGHTDPRSLRHYLKPKED |
| 1625 | WP_069455445.1 |
| | MSKTNRFYPIDVNQQPVGVNTHLTKNLTQAGNAFFEESALPQSVHNDFYNAAAETEFEISSNTRRVYQTS |
| | FSLFAQYCLEHRLQSLPTDPRSVISFIGHQKELLMADTGMQLSKQTLTTRLAAIRYYHIQAGFPSPTEHP |
| | LVLRVMRGLSRNHNRRVQDYDQQPIMYDEVELLLQAVEQQPHPLLRSRDKAIIQLGLQGGFRRSELANLK |
| | VQYLSFMRDKLKVRLPFSKSNQQGLREWKNLPDSEPFAAYHAVKAWLHESQISDGHLFRSISRDGKTLRP |
| | YQVKDNNKSNTTFNRNSGFLNGDDIYRIIKQYCVKAGLPAQYYGAHSLRSGCVTQLHENNKDTLYIMART |
| | GHTDPRSLRHYLKPKED |
| 1626 | WP_050991348.1 |
| | MSKTNRFYPIDVNQQPVGVNTHLTKKLTQADNAFFEESALPQSVHNDFYNAAAETEFEISSNTRRVYQTS |
| | FSLFAQYCLEHRLQSLPTDPRSVISFIGHQKELLMADTGMQLSKQTLTTRLAAIRYYHIQAGFPSPTEHP |
| | LVLRVMRGLSRNHNRRVQDYDQQPIMYDEVELLLQAVEQQPHPLLRSRDKAIIQLGLQGGFRRSELANLK |
| | VQYLSFMRDKLKVRLPFSKSNQQGLREWKNLPDSEPFAAYHAVKAWLNESQISDGHLFRSISRDGKTLRP |
| | YQVKDNNKSNTTFNRNSGFLNGDDIYRIIKQYCVKAGLPAQYYGAHSLRSGCVTQLHENNKDTLYIMART |
| | GHTDPRSLRHYLKPKED |
| 1627 | WP_055647363.1 |
| | MSKTNRFYPIDVNQQPVGVNTHLTKKLTQADNAFFEESALPQSVHNDFYNAAAETEFEISSNTRRVYQTS |
| | FSLFAQYCLEHRLQSLPTDPRSVISFIGHQKELLMADTGMQLSKQTLTTRLAAIRYYHIQAGFPSPTEHP |
| | LVLRVMRGLSRNHNRRVQDYDQQPIMYDEVELLLQAVEQQPHPLLRSRDKAIIQLGLQGGFRRSELANLK |
| | VQYLSFMRDKLKVRLPFSKSNQQGLREWKNLPDSEPFAAYHAVKAWLHESQISDGHLFRSISRDGKTLRP |
| | YQVKDNNKSNTTFNRNSGFLNGDDIYRIIKQYCVKAGLPAQYYGAHSLRSGCVTQLHENNKDTLYIMART |
| | GHTDPRSLRHYLKPKED |
| 1628 | WP_112352796.1 |
| | MNKLSINQYHPRQVTSDKSFFEETELPISILDDFKSAASETEYELAPNTRRVYRASFNIFTQYCQHHGLS |
| | NLPADPRAVISFIGHQKEQVQQKTGMQFSKQTITTRLAAIRFYHIQAGFHSPTEHPLVIRVMRGLSRNKH |
| | RLTKDYDQQPIMYDELELLLQTIDKQGQELTRARDKAIIQLGFQGGFRRSELADIQVNHINFMRKKLKVR |
| | LAYSKSNQQGHKEWKDLPESELFSAFSAVKHWLQVSQLTQGHLFRSLSRDGQRLRPYSVANKSSVDSYAN |
| | PPQVNRGFLRGDDIYQMIKKYCAKAGLSPEFYGAHSLRSGCVTQLHENDKDHLYIMARTGHTDPRSLRHY |
| | LKPKD |
| 1629 | WP_105252541.1 |
| | MSKMIRTNSNAQNNTNVTNERVTGSDHHNNNRAEQPRFFEETFLPQSVRSDYLSAAEETEYEISANTRRV |
| | YNTSFSLFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALNAVKNWLSLANIEDGHLFRSLSRDGK |
| | YLRPYQIVEHHSEANSSLHKNSGFLTGDDIYRIIKKYCTKAGLPAKFYGAHSLRSGCVTQLHENDKDHLY |
| | IMARTGHTDPRSLRHYLKPRD |
| 1630 | WP_012089273.1 |
| | MSKMIRTNSNAQNNANISNERATGSDHHHNNRAEQPRFFEESFLPQSVRSDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDVVKNWLSLANIEDGHLFRSLSRDGK |
| | NLRPYQMKDRHCGSSSLLNKNSGFLTGDDIYRIIKKYCTKAGLPAKFYGAHSLRSGCVTQLHENNKDHLY |
| | IMARTGHTDPRSLRHYLKPKD |
| 1631 | WP_071939473.1 |
| | MSKMIRTNSNAQNNTNVSNERANESGHHHNNRAEQTRFFEETFLPQSVRSDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | NLRPYQMKDRHSGSSSLLNKNSGFLTGDDIYRIIKKYCTKAGLPAKFYGAHSLRSGCVTQLHENNKDHLY |
| | IMARTGHTDPRSLRHYLKPKD |
| 1632 | WP_014358005.1 |
| | MSKMIRTNSNAQNNTNISNERATGSGHHHNNRAEQPRFFEETFLPQSVRNDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | NLRPYQMKDRHSGSSSLLNKNSGFLTGDDIYRIIKKYCTKAGLPAKFYGAHSLRSGCVTQLHENNKDHLY |
| | IMARTGHTDPRSLRHYLKPKD |
| 1633 | WP_106650561.1 |
| | MSKIIRTNTNAQNNTYMSNERATESEHHQNNRAEQPRFFEESFLPQSVRSDYLSAAEETEYEISVNTRRV |
| | YNTSFSVFSRYCAEHQLQVLPADPRSVISFIGHQKELIQESTGVQLSKQTLTTRLAAIRYHHIQAGFHSP |
| | TEHPLVIRVMRGLSRNQSRYVSDYDQQPIMYDEVEMLIQAIDEQEQPLTRARDKAIIQLGLQGGFRRSEL |
| | ADIKVQYVSFLRNKLKVRLPYSKSNQQGLREWKDLPDHEPFAALDAVKNWLSLANIEDGHLFRSLSRDGK |
| | NLRPYQMKDRHSGASSLLNKNSGFLTGDDIYRIIKKYCTKAGLPARFYGAHSLRSGCVTQLHENNKDHLY |
| | IMARTGHTDPRSLRHYLKPKD |
| 1634 | WP_076411519.1 |
| | MSKLTQHLPNSFVSNNGHQQKLTEDNLFFEEQALPISILDDFKSAASETQYEISYNTRRAYQTSFNIFSR |
| | YCEQHGLNTLPADPRSVISFIGQQKELINQKTGAQLSKQTLTTRLAAIRFFHIQAGFHSPTEHPLVLRVM |
| | RGLSRNQLRVTSDYDQQPILYDELELLIQTIDNQKQTLTKARDKAIIQLGFQGGFRRSELASIQVSHVNF |
| | LRNKLKVRLAYSKSNQQGHKEWKDLPEAEPFSAMSAVKLWLDESQIKQGHLFRSLSRDGESLRPYFQAKS |
| | DLDQDAGVQKNSGFLRGDDIYQIIRKYCHKAGLSSDLYGAHSLRSGCVTQLHENDKDHLYIMGRTGHTDP |
| | RSLRHYLKPKD |
| 1635 | WP_012325003.1 |
| | MNKMTPFQTGSLLSRPANTEEKQFYEERELPLSILDDYKSAASETEYEISANTRRAYTSSFSLFSNYCSE |
| | HRLNTLPADPRTVISFIGYQKELIQSRSGAQLSRQTLTSRLAAIRYFHIQAGYHSPTEHPLVIRVMRGLS |
| | RNKQRTVSDYDQQPIMYDELEMLLNVIELQPHAITRARDKAIIQLGFQGGFRRSELADIRVNHLSFLRDK |
| | LKVRLPYSKSNQQGQREWKNLPQSEPFAAFDAVKHWLTVSKIQDGHLFRSLTRDGRQVRDYSVATQGIES |
| | KKRNSGFLRGDDIYQMIRKYCTKAGLSHEFYGAHSLRSGCVTQLHENDKDHLYIMARTGHTDPRSLKHYL |
| | KPKD |
| 1636 | WP_101090209.1 |
| | MSGKRISPISNKALKTVSDDGFYQEHELPLSILNDFESAAKETRYEISHNTRRVYQSSFGIFVTYCESHG |
| | LSSLPADPRSVISFIGHQKDIYQANSGHQLSTQTINSRLAAIRFFHIQSGSPSPTEHPLVIRVMRGLMRN |
| | QNRTVADYDQQPIMYDELELLIQTIDERNQNLTKKRDKAILQLGFQGGFRRSELANIKVNHLSFLRDKLK |
| | VRLPYSKSNQQGQREWKVLPKEEPFSAFDAVKEWLSAAEIKEGHLFRSLTRDGNQIRDYQITDTNLGKGF |
| | LRGDDIYQLIKRYCNKAGLDPQYYGAHSLRSGCVTQLHENKKDHLYIMGRTGHTDPRSLNHYLKPNE |
| 1637 | WP_115136967.1 |
| | MNNQVPEQYHQESNLPSSILDDFHNAAAETEFEVSANTRRNYATSFSIFQDYCQHHGMSALPADPRAVIS |
| | FIGHQKDLYLESGVQLSKATLISRLAAIRFYHLQAGFRTPTDHPMLLRIMRGISRNQYRQQAHYDQQPIM |
| | YTELSRLLSAVDSQQSALLKMRDKALITLGFQGGFRRSELASLQTQHLTFLHDRLRVRLAFSKSNQQGGK |
| | EWKDLPYSEQFAAADYVRRWLEISQLSSGHLFRSISRCGKFTRPYERKMPGSSGRNSGFLNGDDVYRTVR |
| | KYCKIAGLGESWFGAHSLRSGCVTQLHENDKDTLYIMGRTGHTDPRSLRHYLKPK |
| 1638 | WP_064791349.1 |
| | MNKMTPFQTGSLLSRPANTEEKQFYEERELPLSILDDYKSAASETEYEISANTRRAYTSSFSLFSNYCSE |
| | HRLNTLPADPRTVISFIGYQKELIQSRSGAQLSRQTLTSRLAAIRYFHIQAGYHSPTEHPLVIRVMRGLS |
| | RNKQRTVSDYDQQPIMYDELEMLLNVIEQQPHAITRARDKAIIQLGFQGGFRRSELADIRVNHLSFLRDK |
| | LKVRLPYSKSNQQGQREWKNLPQSEPFAAFDAVKHWLTVSKIQDGHLFRSLTRDGRQVRDYSVATQGIES |
| | KKRNSGFLRGDDIYQMIRKYCTKAGLSHEFYGAHSLRSGCVTQLHENDKDHLYIMARTGHTDPRSLKHYL |
| | KPKD |
| 1639 | WP_012142588.1 |
| | MSKSACHTINSILTPNTSIVPSGTNGNSNASDEKFFEETQLPLSILDDYKSAASETEYEISENTRRVYTS |
| | SYAIFNRYCLEHGLSPLPADPRSVISFIGHQKESIQQSSGAQLSRQTLTSRLAAIRYHHIQAGFHSPTEH |
| | PLVIRVMRGLSRNKYRKVADYDQQPIMYDELEMLIDVINQQPQPMTRARDKAIIQLGFQGGFRRSELADI |
| | QVNHLSFLRNKLKVRLPYSKSNQQGQREWKDLPQTEPFAAFDAVKEWIEVSKIKQGHLFRSISRDGSQIR |
| | PYSVSDTTNRKINQTSMDNEELPLSRSNRNCGFLRGDDIYQMIKKYCARSGLSPEFYGAHSLRSGCVTQL |
| | HENDKDHLYIMARTGHTDPRSLRHYLKPKD |
| 1640 | WP_126520563.1 |
| | MVPSGTNGNRNASDEQFFEETQLPLSILDDYKSAASETEYEISENTRRVYTSSYAIFNRYCLEHGLSPLP |
| | ADPRSVISFIGHQKESIQQSSGAQLSRQTLTSRLAAIRYHHIQAGFHSPTEHPLVIRVMRGLSRNKYRKV |
| | ADYDQQPIMYDELEMLIDVINQQPQPMTRARDKAIIQLGFQGGFRRSELADIQVNHLSFLRNKLKVRLPY |
| | SKSNQQGQREWKDLPQTEPFAAFDAVKEWIEVSKIKQGHLFRSISRDGSQIRPYSVSDITNRKINQTSMD |
| | AKEHSLPRLNRNSGFLRGDDIYQMIKKYCARSGLSPEFYGAHSLRSGCVTQLHENDKDHLYIMARTGHTD |
| | PRSLRHYLKPKD |
| 1641 | WP_108946565.1 |
| | MKGQIQFNQALVSQQHVDNDSSEKFFQEQQLPISILDDFKSAASETQYEISANTRRVYQSSFAIFKSYCE |
| | LHNLSALPADPRSVISFIGHQKEVYQEKSGHQLSKQTINTRLAAIRFFHIQAAHHSPTEHPLVIRVMRGL |
| | MRNQYRHTSDYDQQPITYDELEMLLAVIDQQPQQLTRLRDKAILQLGLQGGFRRSELAEVKIEHISFLRD |
| | KLKVRVPYSKSNQQGQREWKDLPKHEDFSAYDAVQHWLDATKLKQGHLFRSLSRDGNSIRDYQITQGKNG |
| | KGFLKGDDIYQMIKKYCDKAGLNSRFFGAHSLRSGCVTQLHENDKDHLYIMARTGHTDPRSLRHYLKPKD |
| | GYS |
| 1642 | WP_037411215.1 |
| | MKGQIQFNQALVSQQQVDSDSSEKFFQEQQLPISILDDFKSAASETQYEISANTRRVYQSSFAIFKSYCE |
| | LHNLSALPADPRSVISFIGHQKEVYQEKSGHQLSKQTINTRLAAIRFFHIQAAHHSPTEHPLVIRVMRGL |
| | MRNQYRHTSDYDQQPITYDELEMLLAVIDQQPQQLTRLRDKAILQLGLQGGFRRSELAEVKIEHISFLRD |
| | KLKVRVPYSKSNQQGQREWKDLPKHEDFSAYDAVQHWLDATKLKQGHLFRSLSRDGNSIRDYQITQGKNG |
| | KGFLKGDDIYQMIKKYCDKAGLNSRFFGAHSLRSGCVTQLHENDKDHLYIMARTGHTDPRSLRHYLKPKD |
| 1643 | 01040422.1 |
| | MDKYISRFTNYLKVEKNYSGHTVKNYLVDLKAFKGFAQDTDIAKIDHLFLRRYLASMRSSGYSKRTIARK |
| | LATLRSFFRFLCTDGYLKDNPISGISTPKLDKKLPIFLDVDTVFRLLESPGRDISGLRDRAIMETLYSTG |
| | IRVSELAGLKMENVDFIGEVIKVFGKGRKERMIPIGNKAVNSIRAYMDERGRLGIDRKELFLNKSKRPLS |
| | IRGIRRVIDKHIKNTSAKEHVSPHTLRHSFATHLLDRGADLRSIQELLGHMNLSTTQIYTHVTTERLKSV |
| | YDKTHPRA |
| 1644 | WP_047914882.1 |
| | MNIEKIARKGKPTVEKRTKQDGSISYRYTGYYLGIDEVTRKKVNATITGQTLKELDRNMIKARLDFERNG |
| | HTKKEQLQITLFSELAEEWFVSYKLITSSENTNNRVRGYLDTYIIPRFGDYLPDKIKPIDVQKWVNECAA |
| | KARQVAAEGRRAKKGEAKDFGAALYKLRDIFDYGITNFGLKKNPATTVQVPPKPKENKVKVKVLHDDELK |
| | IWLKHLSSLPNNQANRRFKLICETLLASGIRINELLALTIDDLNFETSELDINKTLMWKAADKKTGIKGK |
| | VICKPSPKSDAGCRKVDVPPKILERLKAWHDEVSERFEKIGLDKPSLIFPTVYGAYMCDRNERTTLKKQL |
| | TACGLPLYGFHIFRHTHASLLLNAGTNWKELQVRMGHKSIATTMDLYAELAPKKKAEAVNIYLDKIDELT |
| | A |
| 1645 | WP_010729268.1 |
| | MPTKLSNGKYKTNLRYPKRFKEITGIASEKYQKTFPNRQLAIKAENDMKKKIEKVLREENANSLELKGKI |
| | TFKKFYESKWLPRYELGQTIRSNRPPSDITISNTKDIFRLHILPMFGEYAMNYLNINTEIISDELTKKSK |
| | EYANIKIIKGYVRSMFDIAEILNYIEFNRTTKIIQSITAPKKNALEEKRIQEGKQALSSKELTNWIEAVN |
| | DDFNNHLLTFHDYTLFMITLYLGDRKSETYALQWKYIDFEKQTVRLKHTLDKYQRKKFTKGRKDTVIQVP |
| | EVVMTLLSEWKSVQADQLLKLKIKQTLDQYLFTYTKPSGEVNCPVHADYLNYRINSIKRRHPDLVHLSPH |
| | KLRHTYATIARQGGADMNQISNALTHSDISTTKIYVNTPDIVDKAVFEAFQRGLNKCD |
| 1646 | WP_003171984.1 |
| | MRSEDIPLFLKTSYQYNYIYYIFFKALLNTGMRKGEAAALQWKDINLKEHTIIISKTLDFTAKTKEELFG |
| | dtktftskrtimipkslvdellahkkwqnanklvlqdayeheldlvfsrvdgkflpkstlfnafsrilkk |
| | ANLPRLEIHSLRHTHAVLLLESGASMKYIQDRLGHKSIEITSNVYSHISDKINKDSISGFEAYMNNVLG |
| 1647 | WP_033660184.1 |
| | MRSEDIPLFLKTSYQYNYIYYIFFKALLNTGMRKGEAATLQWKDINLKEHTITISKTLDFTAKTKEELFG |
| | dtktftskrtimipktlvdellahkkwqnanklvlqdayeheldlvfsrvdgnflpkstlfnafsrilkk |
| | ANLPRLEIHSLRHTHAVLLLESGASMKYIQDRLGHKSIEITANVYSHISDKINKDSISGFEAYMNNVLG |
| 1648 | WP_002076880.1 |
| | MCSSYQRAPDLVKTSYQYNYIYYIFFKALLNTGMRKGEAAALQWKDINLKEHTITISKTLDFTAKTKEEL |
| | FGDTKTFTSKRTIMIPKSLVDELLEHKKWQNANKLVLQDAYEHELDLVFSRVDGNFLPKSTLFNAFSRIL |
| | KKANLPRLEIHSLRHTHAVLLLESGASMKYIQDRLGHKSIEITANVYSHISDKINKDSISGFEAYMNNVL |
| | G |
| 1649 | WP_016115818.1 |
| | MASFRKYQTKDGAKWLYKIYTTIDPKTGKKKQTTKRGFKTKKEAQLHAAKAETELSNGTFIEDKNVMIST |
| | FLNDWLITYKKGKVRNHTYNLHKTAINKHIVPFFGSYKVFDITPSLCQKFVNHLLEEGYSENSVKNYTAP |
| | LKGALLKAVDLQLIQQTPFRGIVIAKSDTEDKKIKHLEGQEVNTFVQKLKDTEPHYFSLFFTLLHTGIRK |
| | GEALALRWDDIDLEEGTISIRHTFTYDYKNLDNLFAKPKTKASYRTIILADFLIQILKNHKLEQNKCKLK |
| | LGGLYHDLSLVFARENGLPYPKSTLQRAMTRILKKANVTNITIHGLRHTHAVLLLDAGYSMKEVQERLGH |
| | DSIQITSDIYAHISKEMNKKSLNKYEAFAKRNLL |
| 1650 | WP_011736163.1 |
| | MPKRIAPLSDLQVRNAKPKEKQVTLFDGGGLYLLITPTGGKLWRLKYSLFGKEKLLALGTYPEISLADAR |
| | QRREDARKQVANGIDPGEVKKAQKVSSGEGDENSFEVIAREWHGKFLLNKSESYRDKMLSNFERDVFPWI |
| | GRVAVKNLKAPELLSALRRIEGRGALETAHRTRSACSQVLRYAVATGRAERDCASDLIGALPPYKKGHRA |
| | ALTDPKEVAPLLRAIDDYQGTFPVKCALKLAPLLFVRPGELRKAEWSEVDFKATEWRIPGDKMKMKNDHI |
| | VPLASQAVEILKELYPLTGHSKFLFPSPRSPLRPMSDNAILSALRRMGFEKDEMSGHGFRAMARTILDEV |
| | LHVRPDFIEHQLAHAVRDPNGRAYNRTAHLAERKKMMQAWADYLDDLKTRNGI |
| 1651 | WP_044402340.1 |
| | MAKVTVRKETGKLVMDFTYCNVRCREQTALPDTLQNRKRVEAVLEKIKKALKNGTFQYRDYFPESALASR |
| | FDQATTVDAGKAMQSPVNSPSPLFQDFATQWFKEHEIEWRRSHIRSLRSTLDGRLIPHFGQKVVSSITKS |
| | DILAYRATLAKVKGRGDKEGLSPKRINEIIGTLCQIIDEAADRFEFTTPTTNIKRLRVRKVDVDPFSLQD |
| | VQSILATVRADYRNYFTVRFFTGMRTGEVHGLKWRYVDFERRLIRVRETVVLGEDEYTKTDGSQRDIQMS |
| | QPVVEALTKQYEVTGKLSDYVFCNLMGAPLDNKNFTDRVWYPLLRHLGLTERRPYQMRHTAATLWLASGE |
| | APEWIARQLGHTSTEMLFRVYSRYVPNLTRQDGSAMERLLASRLATGKVLRMDRAHLQQVGDSNLFAEAG |
| | GSERATMPVPKPRGVAVGALERARTNWSRTSQDITLPERHAGEDPQPPPPGAMRTHVRRLNPLHA |
| 1652 | WP_008400148.1 |
| | MAKGSVRKKGKKWYYRFYVEDASGNLVQKECVGTESKSETEKLLRQAMDDYEKKKFVAKAENLTVGQLLD |
| | VWAEEELKTGTLSNGTVENYLGTIRNIKKHPLAERKLKNVTSEHLQSFFDLLSFGGVHPDGKERKGYSKD |
| | YIHSFSAVMQQSFRFAVFPKQYITFNPMQYIKLRYQTDEVDLFSDEDMDGNIQPISREDYERLLAYLQKK |
| | NPAAILPIQIAYYAGLRIGEACGLAWQDVNLEEQCLTIRRSIRYDGSKRKYIIGPTKRKKVRIVDFGDTL |
| | VEIFRNARKEQLKNRMQYGELYHTNYYKEVKEKNRVYYEYYCLDRTEEVPADYKEISFVCLRPDGCLELP |
| | TTLGTVCRKVAKTLEGFEGFHFHQLRHTYTSNLLANGAAPKDVQELLGHSDVSTTMNVYAHSTRDAKRKS |
| | VRLLDKVVGND |
| 1653 | WP_056871537.1 |
| | MSAYINSKISNWEMFMLHQQSLGKPTTLNIAIGYFKGNAEVTLFGFWEEQLRLWEYEKKPATIKSYKSTL |
| | NILRHFNNKLNFGDLTYDCIQKFDLYLRKERNNATNGCFVKHKCLKHMIKESISKGFMDKSPYEHFKVRS |
| | TKGTRMFLTIDEVNAIDDLQISKDNTFLQKSKDLFLFSCFTGLRYSDVVNLTWGNIKQNPDRIEIKIIKT |
| | EKPLLVPLISKAKDILNKYSKLTIKTDSLKALPQQANQVVNRNLKEIMMLAGIKKSISFHCARHSFASNL |
| | VEMNTPILYVKDLLGHQKIEQTMIYAKSIVGNLFDSMNNLNEKYHHVNKHVG |
| 1654 | WP_002990881.1 |
| | MSTKIWQNTLQSYIHYLKLERGLAENSIESYELDLLKFVQFLSHSEIEVAPKNVTPAHVNEFVYQLSTVL |
| | APTSQARIISGLKSFFTFLLVDGQIEKAPTDLLEIPKLGRKLPEVLAMEEIDALLATLDLSTNEGYRNKV |
| | MLELLYSCGLRVSELVNLRLSDLFFEEGFIRVIGKGSKHRFVPIDPDTMELIIMYKQSIRNHMQVKKEDT |
| | DIVFLNRRGGRLTRAMIFTIVKQAAQEANIQKNVSPHSFRHSFATYLLENGADIRMIQLMLGHESILTTE |
| | IYTHISREKLKGVMDRYHPRSRQ |
| 1655 | WP_041890631.1 |
| | MNITLKQRKLPSGRISLLIEYTKGVEVTSTGKKKYIREFENLKLFLHGAPNSPKERKENKEALQMAENIL |
| | AIRQSENLRGKYGIKNKHKGQRCFLDFFLEKTEEKYESPKNYGNWTASFLHLKRCISPTLTFDEVDDDFL |
| | KRVREYIDKKALTKSKLPLSLNSKYSYLNKFRAALRLAFEEGYLTINYAQKVKSFKQAESQREYLTFNEV |
| | QRLVETDCKYEVLKRAFLFSCLTGLRWSDINKLVWSEVRDEDDVCRVIYRQEKTEGVEYLYISKQARELL |
| | GERESLNQLVFTNLKYSAIYNNEIVRWCNRAGIHKHITFHSARHTNAVLLLENGADIYTVSKRLGHREIR |
| | TTQIYAKIIDSKMKEASELIPELKFGE |
| 1656 | WP_011279365.1 |
| | MSRDRAVQQRQPKALAVDDEPAYMTEFRQAMLARGLATRTRNAYVRDLRSCELTNHAALTRWQPEDVLCC |
| | LSILTQDGKTPRTQARMLSSLRQFYLWMIASNLREDNPCERIKSPKLGRPLPKDLAEADVDNLLAAPDSS |
| | TALGLRDKAMLEVLYACGLRVSELVNLSLEQVNLNSGWLQITGKGNKTRLVPLGEYASDALEDYLTHGRG |
| | DLIAHLKAGNCQAVFLTAQGGYMTRQNFWYLLKKYAKVASIDKALSPHTLRHAFATHLLNHGADLRSVQL |
| | LLGHSNLSTTQIYTHVATARLQKLHAEHHPRG |
| 1657 | YP_O09221649.1 |
| | MNAFIRKRNKNYVVYLEFRDDESGKRKQKNMGAFDKKRDANKRLAEVKDSIYKDSFLVPNEITLAGFLLD |
| | FLEKYKDNISASTYKSYIAICKNHINPSIGKYRLQELRNIHIQNYIDDLAGNLNPQTIKVHINVLRLAIK |
| | RAYRIKLIKENIIDGIESPRIKKFKNEIYDKEHMLKLLEVAKGTNLELPISLAIGLGLRLSEVLGLTWDN |
| | IDFDENTITVNKITSRLDGSVILKEPKTESSVRKIFAPIELMNLLKNYRLEQNKKLLRSIVRNEYNLLFF |
| | DRKGNPIAEDVMSKKFRKFLENNDLPHIRFHDLRHSHVTLLINSKVPIKVISERVGHSNINTTLNVYSHV |
| | LKEMDKEASDRISENLFKAN |
| 1658 | WP_076384767.1 |
| | MDKAQRYLTAGTRENTRKSYRAAVEHFEVTWGGYLPATAEGIVRYLAEYAETLSLSTLRQRLAALAQWHV |
| | SQGFPDPTKAPHVRQMLKGIRVVHPTRQKQAAPLQLRHLEKAVNWLNSKAADAVESGDYRALMRYRRDAA |
| | LLLIGFWRGFRSDELARLQVEDTQAEAGIGITFYLPYTKADRDHQGSTFHTPALKKLCPVEAYINWITVA |
| | GMTRGPVFRKLDRWGNLSEKGFKSTSLIPLLRRILEEAGIPAQSYSSHSMRRGFATWASANGWDIKGLMS |
| | YVGWKDMKSALRYVDASNSFGGLAAFSPGRIDHDDPESSS |
| 1659 | WP_017135669.1 |
| | MVEVASKADRYLEANIRENTSKSYAAALTHFEVTWGGYLPTTTESVVRYIAEYADQLALSTLKQRLAALA |
| | NWHQSNGFPDPTKAPKVRQLLKGIRAVHPVQQKQAAPLALLHLEKAVAYLEDEVAQARAVGNMAALLKGT |
| | RDIALLTIGFWRGFRGDELARLTIENTHAERYVGIRFYLGSTKGDRQNIGREYKTPSLSKLCPVEAYLTW |
| | IEAAGLTRGGVFRAIDRWGNISDCPIAAHSLIPLLRDTLDRCGLPSEIYSAHSIRRGFATWAASSGWDIK |
| | TLMEYVGWSDMKSALRYVEPAQQFGGLIRKLEG |
| 1660 | WP_102605325.1 |
| | MTANNKDEGVPSILFGERAAQARTHGTLATPEQLAQQHQRFLAAATSDNTRRTYRSAIRHFQAWGGGLPC |
| | DEALVIRYLLAFAEVLNPRTLALRLTALGQWHRYQGFPDPAASATVRKTLRGIERVNGRPRQKAKALLLG |
| | DLELIVAHLDTLEGLAALRDSALLQVGYFGAFRRSELVTLEVRDLQWEREGLRITLPRSKTDQEGEGLER |
| | AIPYGDSLCCPAKALRSWLEAAQIEQGPLFRRISRWGVVGKVALYEGSVNSILAARAGAAGLLYVPEMSS |
| | HSLRRGLATSAYRAGADFLEIKRQGGWRHDATVHSYIEEARAFEENAAGSLLRRKPST |
| 1661 | WP_002827782.1 |
| | MKLPKGIDLMPSGKYKATASIGSGNTRKRKSKSFTTVSDAKAWLLEMNADMHSGTTYVGDDAKITDAYNE |
| | WVATFVTSKVSPATEKGYYFTGKILAKYCEGWLVKTLDRRHCQKLFNQLIADNYTKNTIKKIKVHVGKYC |
| | RSLVTEGVIKRNPMQEIDIRGARLGKDANQKFISISQYKQLLQALKQRPISQMTPYTMVILVILCTGMRV |
| | SEAIDLRQDDLDEIKLTLRVDSSYSRTVHDSKAPKTKNSYRTIPIPKFLLQRLREWRFEQNRLLMLNGHR |
| | NHEQHLFITKFGNVPDASSVNYYVQKLEHTICQIPVGQTTSTHSLRHTYASYLLSREGGNQSLQYVANVL |
| | GDTQAMVQEVYAHLMPEEKASQANVVRDALEVI |
| 1662 | WP_069552141.1 |
| | MLNVLNITDQLPLVDETLLEPHFLALNAQEAAAAFIAAGTAANTVRSYRSALAYWSAWLQLRYGQALGDA |
| | PLPVAVAVQFVLDHLARPLADGAWAHLLPPSIDTALVAAGIKARLGPLAFNTVSHRLAVLGKWHRIKGWD |
| | SPSEASVLKTLLREARKAQSRQGVNVRKKSAIVLEPLQALLATCTDGARGVRDRALLLLAWSGGGRRRSE |
| | VVGLQVSDVRQLDADTWLYALGTTKTDTTGVRREKPLRGQAAQALAAWLAAAPAESGPLFRRLYKGGKIG |
| | TSGLSADQVARIVQRRAQLAGLEGDWAAHSLRSGFVTEAGRQGVPLGEVMAMTEHRSVTTVMGYFQAGAL |
| | LESRASQLYGEPPPAEVLINKPKVEAD |
| 1663 | AZE17458.1 |
| | MKDYPTLFGRYWNYNTLNVIDITDQLPLVDEIPLDPHALALNAQEAAAAFIAAGTAANTVRSYRSALAYW |
| | SAWLQLRYGQTLGDAALPPTVAVQFVVDHLARPLANGNWTHLLPPSVDAALVAARVKAKPGPLAYNTVSH |
| | RLAVLGKWHRLNGWDSPTEAPALKTLLRDARKAQSRQGITVRKKTAVVVEPLQALLATCSDGVRGVRDRA |
| | LLLLAWSGGGRRRSEVVGLQIGDVRRLDADTWLYALGATKTDTGGIRREKPLRGPAAQALTAWLTVAPAD |
| | DGPLFRRLFKGGKVGTQGLSADQVARIVQRRAQLAGLDGDWAAHSLRSGFVTEAGRQGVPLGEVMAMTEH |
| | RSVSTVMGYFQAGSMLSSRATCLLEDEERHRSDQNA |
| 1664 | SDY43398.1 |
| | MKDYPTLFGQYWNYNTLDVIDITDQLPLVDEMPLDPHALALNAQEAAAAFIAAGTAANTVRSYRSALAYW |
| | SAWLQLRYGQALGDAPLPPTVAVQFVVDHLARPLADGNRAHLLPPSVDAALVAARVKAKPGPLAYNTVSH |
| | RLAVLGKWHRLNAWNSPTEAPALKTLLRDARKAQSRQGITVRKKTAVVVEPLQALLATCTDGVRGVRDRA |
| | LLLLAWSGGGRRRSEVVGLQIGDIRKLDADTWLYALGATKTDTGGVRREKPLRGPAAQALTTWLAAAPAE |
| | SGPLFRRLHKGGKVGATGLSADQVARIVQRRAQLAGLEGDWAAHSLRSGFVTEAGRQGVPLGEVMAMTEH |
| | RSVSTVMGYFQAGSLLGSRATQLLGPTQIASEAALEQTAGSTVFCEPTMTSTGD |
| 1665 | AZD92641.1 |
| | MNVIDITDQLPLVDEIPLDPHALALNAQEAAAAFIAAGTAANTVRSYRSALAYWSAWLQLRYGQTLGDAA |
| | LPPTVAVQFVVDHLARPLANGNWTHLLPPSVDAALVAARVKAKPGPLAYNTVSHRLAVLGKWHRLNGWDS |
| | PTEAPALKTLLRDARKAQSRQGITVRKKTAVVVEPLQALLATCSDGVRGVRDRALLLLAWSGGGRRRSEV |
| | VGLQIGDVRRLDADTWLYALGATKTDTGGIRREKPLRGPAAQALTAWLTVAPADDGPLFRRLFKGGKVGT |
| | QGLSADQVARIVQRRAQLAGLDGDWAAHSLRSGFVTEAGRQGVPLGEVMAMTEHRSVSTVMGYFQAGSML |
| | SSRATCLLEDEERHRSDQNA |
| 1666 | WP_082143226.1 |
| | MGYWRIIPCHNPFNRQCTCHQYEKAPMSDLDRYLNAATRDNTRRSYRAAIEHFEVSWGGFLPATSDAVAR |
| | YLVAHAGVLAVNTLKLRLSALAQWHTSQGFPDPTKAPVVRKVLKGIRAVHPVREKQAEPLQLKHLEQVVA |
| | FLETDALQASATQDPPRLLRAKRDTALILLGFWRGFRSDELCRLSIEHVQAVPGAGISLYLPRSKSDRDN |
| | LGRTYQTPALLRLCPVQAYSEWLSASALVRGPVFRGIDRWGNLGEEGLHPNSVIPLLRQALERAGIPAEQ |
| | YTSHSLRRGFATWAHRSGWDLKSLMSYVGWNDMKSAMRYVEATPFLGMTLATPALI |
| 1667 | WP_110623642.1 |
| | MNVLDITDQLSLVNETSLHPQFLALNAQEAAAAFIAAGTAANTVRSYRSALAYWSAWLQLRYGHVLGDAP |
| | LPAAVAVQFVVDHLARPTADGEWVHLLPASIDAALITAKVKAKPGAQAYNTVCHRLAVLGKWHRLNSWDS |
| | PTEVPALKSLLREARKAQSRQGLSVRKKTAIVLEPLQALLATCTDGLRGQRDRALLLLAWSGGGRRRSEV |
| | VNLQISDVRQLDTDTWLYTLGATKTDTGGIRREKPLRGPAAEALTAWLKAAPAQSGPLFRRMYKGDKVGA |
| | TGLSADQVARIVQRRAKLAGLDGDWAAHSLRSGFVTEAGRQGVPLGEVMAMTEHRSVSSVMGYFQAGALL |
| | ESRATTLLKSSTVGDEGPLKSLYVGANEDAEHP |
| 1668 | RIA35947.1 |
| | MNVLNITEYLSLANETQLDLHSLAINAQEAAAAFIASGTAANTLRSYRSALAYWSAWLQLRYGQALGDAA |
| | LPSSVAVQFVVDHLARPTADGGWAHLLPPTIDAALVAARVKAKLGPLAYNTVSHRLAVLGKWHRINGWGS |
| | PTETVALKALMREARKAQSRHGVSVRKKTAIILEPLQALLATCTDGVRGVRDRALLLLAWSGGGRRRSEV |
| | VGLQIGDVRRLDADTWLYALGTTKTDTGGLRREKPLRGPAALALAAWLEVAPAESGPLFRRIYRGGKVGP |
| | QGLSADQVARIVQRRAQLAGLDGDWAAHSLRSGFVTEAGRQSVPLGEVMAMTEHRSVTTVMNYFQAGSLL |
| | SSQASQLLGPAVGATASAERSDSDSSP |
| 1669 | AZC51718.1 |
| | MNVIDITDQLPLVDEMPLDPHVLALNAQEAAAAFIAAGTAANTVRSYRSALAYWSAWLQLRYGQVLGDAP |
| | LPPAVAVQFIVDHLARPEAGGSWTHLLPPSIDAALVTARVKAKVGPLAYSTVSHRLAVLAKWHRLKDWDN |
| | PGDAPAVKTLLREARKTQTRQGVNVRKKTAIVLEPLQAMLATCTDGVRGVRDRALLLLAWSGGGRRRSEV |
| | IGLLVEDLRRLDANTWLYALGATKTDTGGVRREKPLQGPVAQALAAWLAAAPASSGPLFRRLYKGGRVGS |
| | AGLSGDQVARIVKRRAALAGLDGDWAAHSLRSGFVTEAGRQGVPLGEVMAMTEHRSVSTVMGYFQAGSLL |
| | GSRASQLLPITQEDDGGNSELLTTGDSH |
| 1670 | WP_003452352.1 |
| | MNVLNITEHLSLANETQLDLHSLAINAQEAAAAFIAAGTAANTLRSYRSALAYWSAWLQLRYGQALGDAA |
| | LPSSVAIQFVVDHLARPTAGGGWVHLLPPTIDAALVAARVKAKLGPLAYNTVSHRLAVLGKWHRINGWGS |
| | PTETAALKALLREARKAQSRHGVSVRKKTAIILEPLQALLATCTDGVRGIRDRALLLLAWSGGGRRRSEV |
| | VGLQIGDVRRLDADTWLYALGTTKTDTGGLRREKPLRGPAALALAAWLEVAPAESGPLFRRIYRGGKVGT |
| | QGLSADQVARIVQRRAQLAGLDGDWAAHSLRSGFVTEAGRQSVPLGEVMAMTEHRSVTTVMNYFQAGSLL |
| | SSQASQLLGPAVGATASEERSDSDRSP |
| 1671 | WP_108099739.1 |
| | MNVLEITQQLPLSDEPLLEPHLLAESAQEAAKAFIAGGTAANTVRSYQSALTYWSAWLRLRYGVALGDKA |
| | LPAELVIQFIVDHLARPLEDGSWTHLLPASIDAALVAARVKAKPGPLAHSTVSHRLAVLSKWHRLNDWDS |
| | PVEMPAVKTLLRDARKAQVRQGITVRKKTAVVAEPLQAMLATCTDGVRGIRDRSLLLLTWSGGGRRRSEV |
| | VAMQIGDVRALDADTWLYALGATKTDSSGARREKPLRGQAAVALAEWLAVAPADSGPLFRRMFKGDKVST |
| | LGLSTDQVARIVKRRAKLADLDGNWAAHSLRSGFVTEAGRQGVPLAEVMAMTEHRSVGTVMGYFQVGTLL |
| | NSRATTLLAEPLTPPDQREHEHG |
| 1672 | WP_110637560.1 |
| | MNNTDALQARFDNPLALHEIADTTRAAAEAFIAAGTAVNTVRSYRSALAYWAAWLRLRYGRALGDGALPP |
| | EVAVQFIVDHLARPNADGTWSHLLPANVDAALVAAGVKGKLGALAFSTVSHRLAVVAKWHRLKDWDNPCE |
| | AAAVKTLLREARKAQARQGMAVRKKTAVVLEPLQRMLTTCTDGVRGIRDRALLLLAWSGGGRRRSEVVGL |
| | QIEDLRRLDTDTWLYALGATKTETSGIRREKPLRGPAAQALAAWLAIAPAVSGPLFRRLYKGGKVGTAAL |
| | SADQVARIVQRRAQLAGLEGDWAAHSLRSGFVTEAGRQGVPLGEVMAMTEHRSVNTVTGYFQAGAMLSSR |
| | ATCLLGDEELHRPDQNA |
| 1673 | WP_045217896.1 |
| | MNNTILPLHGEIAPLAVDRLDAEARAAAAAFVAAGTAANTVRSYRSALAYWAGWLQLRYRRHLEDGALPE |
| | AVAVQFLVDHLARPVEGDWQQLLPPALDAALVESGVKAKPGPLSYNTVRHRLAVLAKWHDLKSWPSPTDS |
| | AAVKTLLREARKAQSRQGVSVRKKTAAVREPLEAMLATCTDGVRGLRDRALLLLAWSGGGRRRSEVVGLQ |
| | IGDVRPLDADTWLYALGATKTKTEGVRRELPLRGSAAQALTEWLAAAPATTGPLFRRVYKGGRVGTDELS |
| | GDQVARIVKRRAVLAGLPGDWAAHSLRSGFVSEAGRQGVPLGEVMAMTEHRSIPTVMGYFQAGTLLNSRA |
| | AHLLALPLNTQADASKSSETRQA |
| 1674 | WP_128325317.1 |
| | MNNTLPLDGLPNTPLALHGLADSTRAAAEAFISAGTAANTVRSYQSALSYWSAWLQLRYRRSLGDGALPP |
| | DVAVQFIVDHLARPDGDGNWSQLLPPQLDAALVAAGVKGKLGALAFSTVSHRLAVLAKWHRLNAWDNPCE |
| | ASAVKTLLREARKAQARQGVALRKKTAVVLEPLQAMLATCSDGVRGIRDRALLLLAWSGGGRRRSEVVGL |
| | QVEDLRRLDADTWLYALGVTKTDTGGVRREKPLQGPAAHALQGWLEAAPARSGPLFRRLYKGGRVGSAGL |
| | SGDQVARIVKRRAALAGLEGDWAAHSLRSGFVTEAGRQGVPLGEVMAMTEHRSVNTVMGYFQAGSLLGSR |
| | AANLMGNESVDTERAPDHTTTHTKPNH |
| 1675 | OWK92550.1 |
| | MNNTESLQPSIDTPLAPHELAASTRAAAEAFIAAGTAANTVRSYQSALAYWSAWLRLRYRRALGDAALPP |
| | EVAVQFIVDHLARPGADGGWSHLLPADLDAALVVMGVKGKLGALAFSTVSHRLAVLAKWHRLKQWDNPTE |
| | TPAVKTLLREARKAQVRQGVAQRKKTAVVLEPLQAMLATCSDGVRGVRDRALLLLAWSGGGRRRSEVIGL |
| | QIEDLRRLDADTWLYTLGATKTDTGGVRREKPLQGPAAQALSAWLEAAPACRGPLFRRLYKGGRVAPHGL |
| | SGDQVARIVKRRAAMAGLDGDWAAHSLRSGFVTEAGRQGVPLGEVMAMTEHRSVSTVMGYFQAGALLDSR |
| | ASKLLGSTPGASEPPPE |
| 1676 | WP_024717480.1 |
| | MNNTESLQPSIDTPLAPHELAASTRAAAEAFIAAGTAANTVRSYQSALAYWSAWLRLRYRRALGDAALPP |
| | EVAVQFIVDHLARPGADGGWSHLLPADLDAALVAMGVKGKLGALAFSTVSHRLAVLAKWHRLKQWDNPTE |
| | TPAVKTLLREARKAQVRQGVAQRKKTAVVLEPLQAMLATCSDGVRGVRDRALLLLAWSGGGRRRSEVIGL |
| | QIEDLRRLDADTWLYTLGATKTDTGGVRREKPLQGPAAQALSAWLEAAPACRGPLFRRLYKGGRVAPHGL |
| | SGDQVARIVKRRAAMAGLDGDWAAHSLRSGFVTEAGRQGVPLGEVMAMTEHRSVSTVMGYFQAGALLDSR |
| | ASKLLGSTPGASEPPPE |
| 1677 | WP_101293615.1 |
| | MNNTDPFEVLPIAPLALHGLADSTQAAAEAFIAAGTAANTVRSYRSALAYWAAWLQLRYGRAIGDGALPS |
| | DVAVQFIVDHLARPDADGDWSQLLPAQLDAALVAAGVKGKLGALAFNTVNHRLAVLAKWHRLNDWDNPCE |
| | APTVKTLLREARKAQARQGVALRKKTAMVLEPLQAMLATCTDGVRGVRDRALLLLAWSGGGRRRSEVTAL |
| | RVEDLRRLDADTWLYALGATKTDTGGVRREKPLRGPAAQALNAWLAAAPASSGPLFRRLYKGGRVGSASL |
| | SGDQVARIVKRRAQLAGLEGDWAAHSLRSGFVTEAGRQGVPLGEVMAMTEHRSVTTVMGYFQAGALLESR |
| | ASLLFGESPVAETVNEAPPTDVQT |
| 1678 | WP_031642620.1 |
| | MQPSIDTPLAPHELAASTRAAAEAFIAAGTAANTVRSYQSALAYWSAWLRLRYRRALGDAALPPEVAVQF |
| | IVDHLARPGADGGWSHLLPADLDAALVVMGVKGKLGALAFSTVSHRLAVLAKWHRLKQWDNPTETPAVKT |
| | LLREARKAQVRQGVAQRKKTAVVLEPLQAMLATCSDGVRGVRDRALLLLAWSGGGRRRSEVIGLQIEDLR |
| | RLDADTWLYTLGATKTDTGGVRREKPLQGPAAQALSAWLEAAPACRGPLFRRLYKGGRVAPHGLSGDQVA |
| | RIVKRRAAMAGLDGDWAAHSLRSGFVTEAGRQGVPLGEVMAMTEHRSVSTVMGYFQAGALLDSRASKLLG |
| | STPGASEPPPE |
| 1679 | WP_042948796.1 |
| | MQPSIDTPLAPHELAASTRAAAEAFIAAGTAANTVRSYQSALAYWSAWLRLRYRRALGDAALPPEVAVQF |
| | IVDHLARPGADGGWSHLLPADLDAALVAMGVKGKLGALAFSTVSHRLAVLAKWHRLKQWDNPTETPAVKT |
| | LLREARKAQVRQGVAQRKKTAVVLEPLQAMLATCSDGVRGVRDRALLLLAWSGGGRRRSEVIGLQIEDLR |
| | RLDADTWLYTLGATKTDTGGVRREKPLQGPAAQALSAWLEAAPASRGPLFRRLYKGGRVAPHGLSGDQVA |
| | RIVKRRAAMAGLDGDWAAHSLRSGFVTEAGRQGVPLGEVMAMTEHRSVSTVMGYFQAGALLDSRASKLLG |
| | STPGASEPPPE |
| 1680 | WP_103326070.1 |
| | MSELDRYLQAATRDNTRRSYRAAIEHFEVQWGGFLPATAEGVARYLAAYAGELSINTLKLRLSALAQWHN |
| | SQGFVDPTKAPVVRQVLKGIRAVHPAQEKQAVPLQLQDLERVASWLDEQASQALAERCQAGLLRARRDRA |
| | LILLGFWRGFRSDELCRVQVEHVQAHAGSGISLYLPRSKGDRDNLGRTYSTPALQRLCPVQAYIEWINTA |
| | ALVRGPVFRAIDRWGNLGEQGLHANSVIPLLRQVLEQAGIAAECYTSHSLRRGFATWAQRSGWDLKSLMA |
| | YVGWKDLKSAMRYVEAEPFAGMAQLREKAVAP |
| 1681 | WP_076449657.1 |
| | MLDWKVALEALDGAYSDATMRAYFADVQAYVSWCDETVCDPLPGSVAQICAFIEDQGRNKAPSTVRRRLY |
| | AIRKVHRLLGLPDPTEDEAINLALRRVRRANPVRPQQARGLNASDLERFLAVQPKTPWGLRNAAMLALGY |
| | ELMTRRSELIALRDSDLELRSDGTLRVLIRRSKADQEGQGRLAFTSVKTADRVRFWQEWRGCVTDWLFCP |
| | IYQGQPIDRGLSDTTVKTVIKTAAKRAGFPPEDVRAFSGHSMRVGAAQDLLKRGFDTSAIMRAGGWKSVS |
| | VLARYLEVAEQNVWEM |
| 1682 | WP_074635693.1 |
| | MPQIIERKRKDGSTAYVAQINIRRNGKWAHRESRTFDKHSSASAWFKKRMKEITAAGADLTAINSKGRTL |
| | STAIDRYITESVKEIGRTKAQVLRSIREYDIASMNCNDIQSHDIVQFAKELGATRTPATVGNYLSHLGAI |
| | FAVARPAWGIPLDQQAMKDAFVVCNRLGITGKAKRRDRRPTLDELDALLTMFEDKHRRRPNSLPMHRVVG |
| | FALFSTRRQEEITRVAWKGLDQTHNRVFIKDMKHPGDKVGNDVWYDLPAPAINIAMAMPRKKPLVFPYHS |
| | DTISAAFTRACKVLEIEDLRFHDLRHEGVTRLFETGETIPQVAAVSGHRSWSSLQRYTHIKQTGDKYEDW |
| | KWLQRLTTSN |
| 1683 | WP_034633966.1 |
| | MGVILMKVITLLTKEGKTRYMLLDHNNEPVQPVLHYLKFKDNSGASRNTLRSFSYHLKLFFEFLEQINKD |
| | YRDIGIDEMADFIRWLQNPHQDVKVSPIFPKQPIRKAKTVNIIINTVLGFYDYLMRHEDYSIQLTERLKK |
| | QVPGSRKGFKSFLHHINKNKSFTSHILKLKVPKQQPKTLSKDQIALIMNACVNMRDLFLIQLLWESSMRI |
| | GEALTLWLEDFEVDARKIHIRDRGELSNLAEIKTVCSPRSIDVSEDLINMYFDYIAQFHTDEVDTNHVFI |
| | KLTGENKGQPLEYTDVVALVQRLRKKTGIYFTPHMLRHTSLTELRKAGWRDEHLMNRAGHAHIQTTMQMY |
| | IHPSDEDIRKDWENAQDRMKINKENKENNE |
| 1684 | WP_012549223.1 |
| | MATVAIEKVIGKKAIKYKARVRLTSNRKRIFEQSKTFTKESEAKNWATKLAKQLNKSGVPTEKQKTILIG |
| | DLITKYLIDPVTSASIGRSKYAVLSRLRAYDIALIQADLLTAHDLINHCRVRKEESTHPLPQTIYHDITY |
| | LKSVIDVAEPMFGYIANTKAHHDAIPTLVRYDLIGRSQRRERRPTNKELVTMEQGLTRRQSHRCANIPLV |
| | DIEHLSIMTCMRLGEITRITWDDVDFKASTLTIRDRKDPRNKHGNNCIIPLYQKVKEIIERQPKVGTLIF |
| | PYKKESIGAAWQRVCKEEGIEDLHYHDLRAEGACQLFERGLNIVEVSKITGHKDINVLNNVYLRLGISEI |
| | HHNLSS |
| 1685 | WP_O16110451.1 |
| | MEFDIIKINNQTSLKQIEKYKKRFANILSLWNDNVLDEAELRKETNERDDKQTYEGFSDEEILYYYLNRQ |
| | THFDKEKRIKDNSRTLYARDLSQFYFFIKQSKEFLQQDVKDYEEGYMWGNLRKRHIRSYQKWLSQEAISY |
| | QSNQRYKPSTVSRKLGIIRSFLKWLYEIQYIQDPLHVEILSTTVAKLHKPKRDLSYEEVKQLLNYYKGNE |
| | INYALVSFLATTGLRIAEVAHAKWKDIEYDSVRNRYYLRVDTKGDDERIVSINKEIFQRIISFRIRRRLT |
| | IDLGNQDGGTIFQTKNHTAYRENYLSQYITKIIKDTGLPFTKNIRITPHFFRHFYVQYLYDYKGLPPHLI |
| | AAAVGHKDDRTTKENYLKQRLTKDSDAGNLIGENEF |
| 1686 | WP_048658860.1 |
| | MILKKNANYYSSLAPNQVFDSERKAMQLEKRLALKLERECAGKDFRTLDELITLWYRMHGKTLRDHIRLR |
| | KSLYRISERLGNPIASDFTSKDFAHYREQRSIEVTTTTINREHAYLRAMFNELERLGVIEFENPLIKIRQ |
| | FKEREKELRYLAHDEIARLLESCQTFSNQSLSFIVKICLATGARWGEAESLKPSQIKNNQITFLNTKSSK |
| | NRTVPINKTLYDELTALESISEERMFLNSLSAFRKAVAEAKIDLPKGQMTHVLRHTFASHYVMSGGNIVK |
| | LRDVLGHSEITTTMRYAHLAPEHLEETLTLNPLNQHQNTTDS |
| 1687 | WP_069945392.1 |
| | MKYHPITDTIELQALQRDILDSQEFKSVYPTLSEYIDSFEQQGIPAKNDLKQLLNFLVTGLSNAKGTQSR |
| | FRNEAERFTLFCWHERGKSVLDIKLEDIKLYIDWIWSPPKNLIADTTISSRFKYRSNSDIRVVNPEWRPF |
| | VHRTPKANRKIESLIAPGKASSIKHAYKLSQTSLRNSYASLNIFFKWLIDAELVMRNYLADAKKNCKYLI |
| | KGKIYQPPHTFDDEVWDIFIQCLTDAADENPKFEIHRFVVLSLKVLFLRISELSSRDYYTPLFNHFRPDP |
| | SNEGWVLHVVGKGKKERVVTVPDSYIENVLGRYRESMDLAPLPRIDEDTPILPSTKTGKPLKQDSVNNIV |
| | EEAFDLVISTLMKSGKKQQALDIAGASSHWLRHTGATQALDELNETMLAEELGHASVKTTVEIYVAPAHR |
| | DRIRKGSQRKL |
| 1688 | WP_085070731.1 |
| | MNELTTDLKLLHEATLNNLKNSKANNTLRAYKSDFKDFGAFCAKNGLNSLPTEPKIVSLYLTHLSKNSKI |
| | STLRRRLVSISMVHKMKGHYLDTKHPIIVENLMGIRRVKGSIQRGKKPLLINHLKLLIDTINEQKTEEIK |
| | KFRDKSLILIGFGGGFRRTELISIDHEDLEFVPEGLKITIKKSKTDQYGEGMIKGIPYFSTENYCPVKNL |
| | NKWLEISKIKSGPIFRRFSKGLSLTDKRLTDQSVVLLMKEYLNLAGIENTNFAGHSLRSGFATVAAESGA |
| | DERSIMAMTGHKTTQMVRRYIREANIFKNNALNKVKF |
| 1689 | OCW82643.1 |
| | MNEITTDLKSLHEATLNNLKSSKANNTLRAYKSDFKDFGAFCAKHGLNPLPTEPKIVSLYLTHLSKNTKI |
| | STLRRRLVSISMVHRLKGHYLDTKHPIIVENLMGIRRIKGSIQKGKKPLLINHLKLIINVINEQKTEEIK |
| | KLRDKSIILIGFGGGFRRTELISIDHEDLEFVSEGLKITIKRSKTDQFGEGMIKGLPYFDNEIYCPVTNL |
| | QKWLEISKIKSGPIFRRFSKGLSLTDKRLTDQSVVLLMKEYLKLAGIENKNFAGHSLRSGFATVAADSGA |
| | DERSIMAMTGHKTTQMVRRYIREANIFKNNALNKIKV |
| 1690 | WP_037412868.1 |
| | MAPFLGPNAKGPKIEGPKMAKIAKKLTDTEIKNTKPAEKEINLFDGDGLMLRIAPLSKGGKKNWYFRYAV |
| | PVTKKRTKMSLGTYPHLTLAKARALRDEYLSLLANGIDPQIHNNDKANALKDATEHTLQAVARKWLDEKV |
| | KTSGISPDHAEDIWRSLERNIFPGLGNVPIKEIRPKLLKQHLDPIEQRGVLETLRRIISRLNEIFRWAAT |
| | EELIEFNPADNLGHRFSKPKKQNMPALPPNELPRFMLAISNASIRLETRLLIEWQLLTWVRPGEAVRARW |
| | SDIDEDNRFWNIPGEFMKMKRPHKIPLSKEAMRILESIKPISGHREWVFPSIKAPLNHMHEQTANAAIIR |
| | MGFGGELVAHGMRSIARTAAEESGKFRTEVLESALAHTKNNEIIAAYNRSEYLAERTELMQWWGDYVQAQ |
| | KYKAIAA |
| 1691 | WP_076591309.1 |
| | MNNVDHYLHAATRENTRKSYQAAVRHFEVEWGGFLPATANSVARYLADHAELLSANTLRQRLAALGQWHI |
| | DQGFPDPTKAPIVRNVFRGIRASHPSQEKQAKPLLLAEVEQVATSLSAFAAQAQEKGDRSLSLRLKRNNA |
| | LLLIGFWRGFRADELTRLAVESISVVPGEGMICYLPHTKGDRQYRGTPFKVPALAKLCPVSAYQDWQNSA |
| | QLTDGPVFRAIDRWGHVGERGMHVDSIGPLLRSILSENGVVSSELYSSHSLRRGFANWAISSGWDIKTLM |
| | SYVGWKDVQSAARYVDAADPFGNHLLSSAS |
| 1692 | WP_013525333.1 |
| | MAPIVDLSRENRPEESKAPSHSTTAIASTSPPSPPADDLPDIVDIVMEMAQAPCEPQNAPPLPAHLEGLA |
| | ERARDYVEAASSANTRRAYAADWKHFCAWARRQHLDVLPPDPQVVGLYITACASGKVTGDKKPNAVSTIE |
| | RRLSSITWNFSQRGQPLDRKDRHIATVLAGIRNTHASPPRQKEAILTEDLIAMLETLDRGTLRGLRDRAM |
| | LLLGFAGGLRRSEIVGLDVARDQTQDGRGWIEVLDKGVLVALRGKTGWREVEIGRGSSDATCPVVAVQTW |
| | LKLARVGHGPLFRRVTGNGKAVAAERLNDQEVARLVKRTALAAGVRGDLSEGDRAEKFSGHSLRAGLASS |
| | AEVDERYVQKQLGHTSAEMTRRYQRRRDRFRVNLTRASGL |
| 1693 | WP_127402674.1 |
| | MAPIVDLSRENRPEESKAPSHSTTAIASTSPPSPPADDLPDIVDIVMEMAQAPCEPQNAPPLPAHLEGLA |
| | ERARDYVEAASSANTRRAYAADWKHFCAWARRQHLDVLPPDPQVVGLYITACASGKVTGDKKPNAVSTIE |
| | RRLSSITWNFSQRGQPLDRKDRHIATVLAGIRTTHASPPRQKEAILTEDLIAMLETLDRGTLRGLRDRAM |
| | LLLGFAGGLRRSEIVGLDVARDQTQDGRGWIEVLDKGVLVALRGKTGWREVEIGRGSSDATCPVVAVQTW |
| | LKLARVGHGPLFRRVTGNGKAVAAERLNDQEVARLVKRTALAAGVRGDLSEGDRAEKFSGHSLRAGLASS |
| | AEVDERYVQKQLGHTSAEMTRRYQRRRDRFRVNLTRASGL |
| 1694 | WP_066605681.1 |
| | MTPALSERWRQHLALDRRRSVHTVRAYVATAERLIAFLEQHRGEGVSPATLAHIDQAELRAFLASRRTDG |
| | IGNLSAARELSAVRGFLRFVGGDDARVPLLKGPRVKRGLPRPISPDEAVALAQDIAETAREGWIGARDWA |
| | VLLLLYGAGLRIGEAMGLHGDILPLGDTLRVTGKRGKTRIVPLLPQVRAAIDAYVDACPYPPARDQPLFR |
| | GARGGPLSPALIRRAVQGARGRLGLSDRTTPHALRHSFATHLLGRGADLRSLQELLGHASLSSTQVYTQV |
| | DAAHLLDIYRNAHPRA |
| 1695 | WP_080957039.1 |
| | MLISPELGSIFSQWGFFAVREEGSPMTDPADRYLRPVQRDSTQRRYQGVLRYFEQGWGACLPASGDTVVR |
| | YLVEHAESLSSSTLGLHLAALAQWHHSHGFDDPTKNAQVRQVLRSIRAQXPRLVKQAEPLPLIELERCVT |
| | GLQQRIASDHPVVRLRASRDQALILMGFWRAFRADELCRLRVEHNALCRGKQLEVFLGSSKTDREYRGQV |
| | VLLPALKRLCPVQAYEDWLAISELQEGPVFRPINQWGHISPLGLKPDGVTYVLREAFACSGLDGAAYTGH |
| | SLRRGFATWANNDSWTTKQLMDYVGWRDVKSAMRYIDTTAPFGDLRR |
| 1696 | KKX62373.1 |
| | MTDPADRYLRPVQRDSTQRRYQGVLRYFEQGWGACLPASGDTVVRYLVEHAESLSSSTLGLHLAALAQWH |
| | HSHGFDDPTKNAQVRQVLRSIRAQXPRLVKQAEPLPLIELERCVTGLQQRIASDHPVVRLRASRDQALIL |
| | MGFWRAFRADELCRLRVEHNALCRGKQLEVFLGSSKTDREYRGQVVLLPALKRLCPVQAYEDWLAISELQ |
| | EGPVFRPINQWGHISPLGLKPDGVTYVLREAFACSGLDGAAYTGHSLRRGFATWANNDSWTTKQLMDYVG |
| | WRDVKSAMRYIDTTAPFGDLRR |
| 1697 | WP_040041154.1 |
| | MPKLQPKQLEAQRAGDNGKTLRDDGGLFGRVRAKADGTVSISFYYRYRFDGKLKDYACGTWPRESLSKIR |
| | TTRDAAKLLVKQHIDPSSHQKVAKQDAKDAVTARLAEIERQKSEALTFQDLFDTWLLDGVRRADGNAELK |
| | RSFNADVLPKLGKKQIKELTEHDLRGVLRAIVTRGANRTAVVMRNNLTQMFVWAEKRQPWRKLLVDGNPM |
| | DLIEIEKVVSTDYDMNNRRERLLAEEEIRELHDIFQRMQAAYDAAPKKRTAAQPVEKTTQCAIWIMLATL |
| | CRVGETSKARWEHINFDTGEWFIPQDDTKGKRSELTVFLSDFALDQFRQLYKLTGHSEWCFPAKNREGHV |
| | CEKSISKQVGDRQCRFKKGKDGNPRKPMKRRRHDDTLVLANGKNGAWTPHDMRRTGATMMQSLGIALDI1 |
| | DRCQNHVLEGSKVRRHYLHHDYAPEKREAWRLLGEQLTLILSASTHNKAPQQSSQREAPSRSH |
| 1698 | WP_004691481.1 |
| | MSTKLKNGQSRYQSKAKVVTIKKFQLSTLRKAADRLNDAAFEKHGDAYLEFPVIIQGDGNPSEIFNLYLL |
| | KKLEQTIQYDFKTFASIAHQLVDFQRFLEDEQLDCLKFHKLKQLNAIFKYRTRLIEQANAGLISASSARG |
| | RINAVVNFYRFLVTEDLVDHQRYGLPFQDVYKYIAVDNEFGARRKMAIKSHDLAIHVPAKAQNSEAILDG |
| | GELSPLTVEEQAVVLKALQKSSLEYQLMFYLALFTGARLQTICTLRIKCLFNRESDNHGFIRLPVGAGTG |
| | VDTKFQKPMTLLIPHWLALDLKIYINSEQAQQRRQKSNYADSDENYVFLTKLGTPFYTSKAEQQELTDKI |
| | KASDSFGARLKLYEGEAVRSYLKGVLLPEIRLIDPQFQSFKFHDLRASFGMNLLESQLQHLPEGHSAMTA |
| | VEYVQARMGHRNISTTLQYLNYKSRLQWRNKIQHEYESSLMKYVMSSVNPVGDFS |
| 1699 | WP_049006636.1 |
| | MTDKTKLVAISRTDDISALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQDDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1700 | WP_104460435.1 |
| | MTDKTKLVAISRTDDISALDALKLLRFRRYNTARSQLRVTSVWSAWCARHGLTPFPVTAVDVERYINGLN |
| | GSVKMATISHFIACLSSVNSSLGFPDFRNVLIKALVQVWRTRENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1701 | WP_004186933.1 |
| | MTDKTKLVAISRTDDISALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1702 | WP_094320139.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTDTFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1703 | WP_032435650.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKISR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1704 | WP_014386529.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1705 | WP_017901102.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1706 | WP_110204872.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHI |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDL |
| 1707 | WP_004197571.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSQGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1708 | WP_087728582.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLKQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1709 | WP_032413233.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDL |
| 1710 | WP_096903742.1 |
| | MTDKTKLVAIARTDDMSALEALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1711 | WP_130953238.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDKRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1712 | VGI65087.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKTVTGQALPFLISDLNILRRSLHK |
| | SNDLRDIRDLAMIWVGFETLLRNVEIRRIKTGDLKWQNDTSCYLLDVMRTKTSLSSNLTFQLSPQCSQHV |
| | RRLIETVEYIDTETFGHRFLFQPVNIHTNRYFPSTSSKLSRGKSIDRMLVKTGFSERLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1713 | WP_085353366.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFQDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLKNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1714 | WP_080922991.1 |
| | MTDKTKLVAISRTDDMSALDALKLLRFRRYHTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1715 | WP_115793642.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SXDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1716 | WP_085354469.1 |
| | MTDKTKLVAISRTDDMSALDALKLLRFRRYNTARSQLRVTSVWSAWCARHGLTPFPVTAVDVERYINGLN |
| | GSVKMATISHFIACLSSVNSSLGFQDFRNVLIKALVQVWRARGNEKKNVTGQGLPFLISDLNILRRSLHK |
| | SDDLRDIRDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLKNESKVSR |
| | EDVGMLSSNSLNQAFARLWGIAGKVGDSNRQSGRYRTWTGHSVRVGGAIELFKAGYSLEKITEMGNWSDP |
| | KMVFRYIRGYLASEKAMVSFMRNHLDDI |
| 1717 | WP_126123982.1 |
| | MTDKTKLVAIARTDDMSALEALKLLRFRRYNTARSQLRVTSVWSAWCARHGLTPFPVTAVDVERYINGLN |
| | GSVKMATISHFIACLSSVNSSLGFPDFRNVLIKALVQVWRARENEKKIVTGQALPFLISDLNILRRSLHK |
| | SDDLRDIQDLAMIWVGFETLLRNVEIRRIKTGDLKWQNDTSCYLLDVMRTKTSLSSNLTFQLSPQCSQHV |
| | RRLIETVEYTDTETFGHRFLFQPVNIHTNRYFPSTSSKLSRGKSIDRMLVKAGFSEGLLTQLQNESKVSR |
| | EDVGMLSSNSLNQAFARLWGIAGKVSDSNRQSGRYRTWTGHSVRVGGAIELFKAGYSLEKITEMGNWSDP |
| | KMVFRYIRGYLASEKAMVSFMRNHLDDI |
| 1718 | WP_107947608.1 |
| | MSKMIRTNSNAQNNANISNERATGSDHHHNNRAEQPRFFEESFLPQSVRSDYLSAAEETEYEISVNTRRV |
| | YNTSFSVFSRYCAEHQLQALPADPRSVISFIGHQKELIQESTGVQLSKQTLTTRLAAIRYHHIQAGFHSP |
| | TEHPLVIRVMRGLSRNQSRHVSDYDQQPIMYDEVEMLIQAIDEQVQPLTRARDKAIIQLGLQGGFRRSEL |
| | ADIKVQYVSFLRNKLKVRLPYSKSNQQGQREWKDLPDHEPFAALDAVKNWLSLANIEDGHLFRSLSRDGK |
| | NLRPYQMKDRHSGSSSLLNKNSGFLTGDDIYRIIKKYCTKAGLPAKFYGAHSLRSGCVTQLHENNKDHLY |
| | IMARTGHTDPRSLRHYLKPKD |
| 1719 | WP_083915996.1 |
| | MTQLPAVSLADTYAREALSKATARAYRADWNHFLDWCESREVSGLPATVQTICDYLASMAETHARATIER |
| | RVVTIAQAHKIKGLPWVSGQPRIRATLRGMFRLHGRPQVKSAAIEVDELRAILSSMPASTVGLRDRAIFL |
| | LTFAGAMRRSEVARLRRQDVVIGKDGLRILVSRSKSDQVGEGHVLAIPRGANMATCPVEALTRWLRAAPA |
| | DDAIFRSIRADGTVLDHPLHPNSIGEIVKRCAARAGVSATSPNERISAHGLRAGCITSLYRKGVSDEAIM |
| | GHSRHRDLKTMRGYVRRGKLMTESPAKELGL |
| 1720 | YP_003856919.1 |
| | MASLRTSSRKDGSTYTSVLYRLNGKQTSTSFDDPVQAVEFKRMVEQLGAAKALEVIETTDAAARHYTLSE |
| | WLRHYLDHKTGVEKSTIYDYEKVVAKDIDPALGPIPLAALTGDDIAKWVQALADRGLKGKTISNKHGFLS |
| | SALNAAVRAGRIPGNPAAGARLPRTEKAEMVFLTREQYAKLHDNITLPWQPLVEFLVASGARWGEVVALR |
| | PSDVNRDASTVRISRASKRTYEKGSYALGAPKTLKSRRTINVDASVLGKLDYSGEYLFTNTVGNPVRHNN |
| | FHANVWQPALKRAGLDVKPRVHDLRHTCASWLIAAGVPLPAIRDHLGHESIKITVDTYGHLDRSSGQIVA |
| | AAIAAQLDPARG |
| 1721 | WP_132978117.1 |
| | MTSVDRYLEAATRTNTRRGYRSAIRHFEEVWGGFLPATADAVARYLADHAESLALNTLRLRLAALSQWHL |
| | EQGFPDPTKASVVRKVLKGIGELHPAREQQARPLQIEELARLDDWLAARIQESANHDEQAARRRATRDRA |
| | LILLGFWRAFRGDELNRLRVEHIQVIPGEGMTLYLPRTKTDHSARGSEFKVPALSRLCPVDAYLDWISLT |
| | QLTEGPVFRRINRWGAVGDEALHPNSLIPLLRKRFVEAGLAMPEHYSGHSLRRGFASWANANHWDVKSLM |
| | DYVGWKDMKSALRYIERPDAFGRERIERALAQT |
| 1722 | WP_048220040.1 |
| | MNIKRFQFNSGESYSILLDDDLLPMYYPNLFVTLYHRNRSDTANTCYKEFEHIKLFYEIMDILDIDIENR |
| | CKRGVFLERNEVEGITGLAKYHSAILKEVNFTFLNNSLKKKKPSPGKIEGARFSPVINKENLVSSKTCYN |
| | RLTTFANYIGWLENMLFHSTQADTKHLFIVLRPKRKERINIIDNHAVKVVDLLDKNGVKIPLENSDYNDD |
| | YRSLSENQLAQIFEVVKVENNRNPWQRSDVRFRNQLLIHLLSSTGMRRGEVIRIKITDLGRSTTTGRYYL |
| | LVRVGEDMEDKRINKPSAKTSGRRVPLHQNLYHMIEEYIIFHRSKITNVEKTPYLLVAHSSGRNQKGDNG |
| | LSLVSVNKICLQISVVVGFTVHPHMFRHTWNDRFSKHVENLIREGKTTEAKAESDRRKLMGWSSESEMGA |
| | RYARKYEEERAIKTGLKLQDKSYNQEDDSQ |
| 1723 | WP_002351552.1 |
| | MPTKLSNGKYKTNLRYPKKFREITGITSEKYQKVFPTRQLAIKAENAMKRKIETVLREENANSLELKGKI |
| | KFKEFYESKWLPRYELGQTTRSKRAPSYVTISNTKDIFRLHILPMFGEYAMNYLNSNTEIISDELTKKSK |
| | EYANIKIIKGYVRSMFDIAEILNYIEFNRTTKIIQCITVPKKIALEERRIREGNQALSSEELIAWIEAVN |
| | TDLNNHLLTLHDYTLFMLTLYLGDRKSETYALQWKYIDFEKQTVRLKHALDKYQKKKFTKGRKDTVIQVP |
| | EIVMALLSKWKSVQAEQLLRLKINQTPDQYLFTYTKPSGEVNCPVHADYLNYRINSIKRRHPELAHLSPH |
| | KLRHTYATIARQGGANMNQISNALTHSDISTTKIYVNTPDVVDKTVFEAFQKGLKN |
| 1724 | ORE41776.1 |
| | MSDVGRYMAAGRRKNTVRSYESGLRHYEVEWKGLLPATVDNVCQYLATYAAELSVPTLKHRLAALSKWHK |
| | ENRFTDPTKDSRVGQVLRGIKAVHPHTPQQAAALGIADLARVVQVLECAAAEANESGDRKTLLQQKRDLA |
| | FLLIGFWRGFRGNELCNLQVQNVHAERGIGLEIVVHGSKGDRANDGVTFSTPALPKLCPVGAYLDWIEAA |
| | ELKEGPVFRSISRWGVVGESALSLTNVSKLLREILERGGLDASKYSSHSLRHGFAHWAANHGWDLAETMD |
| | YVGWSDPKSALRYMPKKTPFERMANSAYSPPAIEHQSKRLK |
| 1725 | WP_012729869.1 |
| | MAALTKTPSGTWKATIRRVGWPTAAKTFRTKRDAEDWARRTEDEMVRGVFIQRAPSEKTTVADALDRYER |
| | EIVPTKKASTQRREGARIRELKANFGKYSLAAVTPDLVSRYRDDRLARGKANNTVRLELALLGHLFNVAI |
| | KEWHIGLIFNPVANIRKPSPGEGRNRRLSESEQAKLLATVDQHSNPMLGWIVRLAIETGMRQSEILGLRR |
| | GQVDLERRVVRLTDTKNNAARTVPLTKLAALVLQNALGNPVRPIDTDLVFFGEPGRDGKRRAYQFTKVWN |
| | GLKKRTGLIDFRFHDLRHEAVSRLVEAGLTDQEVASISGHKSMQMLRRYTHLRAEDLVSKLDAFASSRR |
| 1726 | WP_103422207.1 |
| | MTYPTLSNPAHQSLQTVFDAQLNSRARRFLRSAKADSTLNAYQADTRIFVFWCQLHGLDPLQTTHHDIMN |
| | FLADQADGVLADWVWLDKEEGKGELRNGEPRKPATLVRRLAGIRYAFKQKGIHPMPTEHPEIKEMMRGIV |
| | RLGDNRKRKTGALTLQPLTQVLDGIDTNDLAGLRDHTLLLLMFSGALRRSEAARIEVSDLDFVGQGIRLR |
| | LKPSKHQLHETEIALIPGKHYCPVSALQKWLHKSRISEGPLFRRMNRWGQLMAEPLGPQGINLMIKRRTG |
| | QTIDDLYVSGHSLRRGFITSAVTAGKPMNKIIEVTRHKDMRTLQEYFDDAHKFSDHALDGLL |
| 1727 | WP_085734974.1 |
| | MNYPHIQAQTQQALQSVFDPQLNSRARRFLRSAKADSTLNAYQADTRIFVFWCQLHGLDPLQTTHHDIMN |
| | FLADQADGILADWVWLDKEEGKGELRNGEPRKPATLVRRLAGIRYAFKQKGIHPMPTEHAEIKEMMRGIV |
| | RLGDNRKRKTGALTLKPLACVLDQIDTGNLAGLRDYTLLLLMFSGALRRSEAAKIEVDDLDFVGQGIRLR |
| | LKPSKHQLHETEIALVPGKQYCPVSALARWLKQSRISEGALFRRMNRWGQLMPEPLGPQGINLMIKRRTG |
| | QAIDDLQVSGHSLRRGFITSAVTAGKPMNKIIEVTRHKDIRTLQEYFDDAHKFSDHALDGLL |
| 1728 | WP_048667503.1 |
| | MSHSNLPTRKQSQVTLSHFQSTEKTLQQWEEQKEKLSRFKLNFPDLTDSFDPDWTSLPPVLTHLREFEEH |
| | RGGLSTHTLRMLAFVIRKWDVYCKSKEAYSFPIHAPVLLIWFKELKLSQNIKINTLKQYRAQLSLFHKIM |
| | NTDDITKLPVIATFFKSLPKDEMEITGSQVIELQAKPFRKHHLTHLMEVWGNKKRAVPFRDLAVLTLSYG |
| | TLLREGEVGKIRKKHIKFLENGDLNIERVTSKTTISPEPKRLTGRFSSIVKCYLDTYCTCLDDEDFVFCW |
| | LTVKGSRPAGYRQTPMSGMTIDRIYQRAHEVLLESGDIVISGDGHRDVWSGHSSRVGALQDGYHAGLSLT |
| | QLIQLGDWKSNEMVLRYLRGLDNDMSPNVLLQKK |
| 1729 | WP_076499665.1 |
| | MKKLDLKNDCIGKNPIIRIEFPYDFELKELVKQFPGCNWDIKKKVWWVSYADNRLTELITFFKGKVWLDY |
| | SQLKKVEIPKAQPVLPPLIASLEIEINKFEDWMRNKRYSESTIKTYKDAVIIFLRFLENKAIVEIENEDL |
| | EKFNKEYILARGYSSSYQNQVVNGIKLFFQNRRGIKFNPEIVYRPKREKLLPNVLSKEEVKSILDSHQNL |
| | KHRAMLCLIYSCGLRRGELLNLKIMDIDSKRNILIIRQTKGKKDRVVPLSPKIIELLREYFKAYKPTIYL |
| | FEGHKSQGKYSEKSLESVLKQALVKSGIKKPVTLHWLRHSYATHLLERGTDLRHIQELLGHNSSKTTEIY |
| | THVSIRSLQKVWSPFDDL |
| 1730 | WP_045829269.1 |
| | MKDISTIPSLAQPAASLALPIHLAQQAADAVRELLAEAAADNTTRSYASALRYWAGWHAARYGVDMTLPV |
| | PEATVLQFVVDHVVRRSSDGELVWELPPSVDQALVAAGLKAKRGPWTLATVRHRVAVLSTAHRLKHVTNP |
| | CEQPAIRTVLSRAARAAVKRGERPRKKTAITIAELEAMLATCDDSIEGLRDRALLCFGFASGGRRRSEVA |
| | AADLRDLRRIGSQGFIYRLEHSKTQQAGVTPTSTPDKPVLDRAARALEAWLAAAGIIEGAIFRRLWKQRV |
| | GPALSPAAVGEIVQRRARLAGLEGDFGGHSLRSGFVTEASRQGVALPAIMQLTEHRSVSSVIGYFQMGGA |
| | TENPAARLLEDG |
| 1731 | KJV34819.1 |
| | MREPRAALATSVDQVLTRLASWSTDFAAGSASATVKAVRSDWAQYLVWCDTSGNSPLPASVLQLEAFLVD |
| | AIDRGRKRATVDRYLYTVGLVHAAAGLPSPSKDPDWSVKWKKLTRRLKTTGGHMRKQAAELDMGGVSAIL |
| | ATLGDSPRDLRDAALLSLASDTLCRESELVAIEVAHLHLNRRRNTWSLHVPFSKTNQDGESPDFRFVSQE |
| | TIVRVRAWQATSGITEGALFRPVGGRPKLAGDASPALLPQEVARIFRRRAKAAGLEGAAAISGHSARIGS |
| | ANDLAENGATSTQIQQAGGWKTERMVTHYTRKSLAGRGAMQDLRRTDPAKTSD |
| 1732 | WP_073285721.1 |
| | MSTVPVVPPSPASTSLAHLAEQTARYVEAGLQGAPNTARAYAGDWRRFTAWCTEHGQVALPASVDTLAGF |
| | VTHLAEAGKKVATIQRHCAAISKAHALRDLASPTDDKKFKVLLEGISRVKGTRQKQAPAFSLANFKRTVK |
| | HIDASTPGGLRDRVILLLGFTGAFRRSELSALDLEDLTFSDDGLTIDLKRSKTNQLGEAEEKAIFYSPDP |
| | SLCPIRTLQAWMRMLGRTAGPVLVSLRKGGRLTERRLTDVHLNKIVQRHLGPKFTAHSLRASFVTVAKLN |
| | GADDSEVMNQTKHKTSSMIRRYTRLDNVRQHNAAQKLGL |
| 1733 | WP_125423373.1 |
| | MSTVPVVPPSPTSTGLAHLAEQTARYVEAGLQGAPNTARAYAGDWRRFSAWCTEHGQVALPASVDTLAGF |
| | VTHLAEAEKKVATIQRHCAAISKAHALRDLASPTDDKKFKVLLEGISRLKGTRQKQAPAFSLASFKRTVK |
| | HIDASTPAGLRDRVILLLGFTGAFRRSELSALDLEDLTFSDEGLTIDLKRSKTNQLGEAEEKAIFYSPDP |
| | SLCPIRTLQTWLRMLGRTTGPVLVSLRKGGRLTERRLTDVHLNKIVQRHLGPKFTAHSLRASFVTVAKLN |
| | GADDSEVMNQTKHKTSSMIRRYTRLDNVRQHNAAQKLGL |
| 1734 | WP_035560163.1 |
| | MSSVPVAPASPSSVGLAHLTEHTTRYVEAGLQGAPNTARAYAGDWRRFTTWCTAHGQVALPASVETLAGF |
| | VTHLAEAGKKVSTIQRSCAAISKAHALRDLASPTDDKKFKVLMDGISRLKGVRQKQAPAFSLASFKRTIK |
| | HIDATTPAGLRDRVILLLGFTGAFRRSELSALDLDDLTFSEDGLTINLKRSKTNQLGEAEEKAIFYSPDP |
| | TLCTIRTLQAWLRLLERTSGPILVSFRKGGRLTDRRLTDVHLNKIVQRHLGPKFTAHSLRASFVTVAKLN |
| | GADDSEVMNQTKHKTSEMIRRYTRLDNVRQHNAAQKLGL |
| 1735 | WP_111480623.1 |
| | MAHLTEHTTKYVEAGLQGAPNTARAYAGDWRRFTEWCTAHAQVALPASVDTLAGFVTHLAEAGKKVSTIQ |
| | RSCAAISKAHALRDLASPTDDKKFKVLMDGISRLKGVRQKQAPAFSLASFKRTLKHLDATTPAGLRDKVI |
| | LLLGFTGAFRRSELSALDMDDLSFSEDGLTINLKRSKTNQLGGAEEKAIFYSPDPALCPIRTLQAWLRLL |
| | ERTSGPILVSFRKGGRLTDRRLTDVHLNKIVQRHLGPKFTAHSLRASFVTVAKLNGADDSEVMNQTKHKT |
| | SEMIRRYTRLDNVRQHNAAQKLGL |
| 1736 | WP_125440609.1 |
| | MAHLTEHTTRYVEAGLQGAPNTARAYAGDWRRFTEWCTTHGQVALPASVETLAGFVTHLAETGKKVSTIQ |
| | RSCAAISKAHALRDLASPTDDKKFKVLMEGISRLKGVRQKQAPAFSLASFKRTLKHLDASTPAGLRDKVI |
| | LLLGFTGAFRRSELSALDLDDLTFSEEGLTINLKRSKTNQLGQAEEKAIFYSPDPALCPIRTLQAWLRLL |
| | ERTSGPILVSFRKGSRLTDRRLTDVHLNKIVQRHLGPKFTAHSLRASFVTVAKLNGADDSEVMNQTKHKT |
| | SEMIRRYTRLDNVRQHNAAQKLGL |
| 1737 | WP_065235645.1 |
| | MRKALMEHALNLLNDKNDKNRKFGVDKVFHPLDVHFDTNSLSAWLSFYFQVHVKGAPEKTVQAKQKDLSK |
| | FLNFFQVEVGHDQVDNWTPAVSKQFQRSLCNTISEKTGKAYKATSINRTMATIRHAGRWLYQQRPLIAGD |
| | PLSGVKDLQTDAPEWNGLNSKQLMRLKAACEQRAKICTRKNQNALLETAVFYVLLGTGLRESELVSLNVH |
| | QYHSKGLHSVVRHKSKRVTTKIPLPQESREHLEHYLKSRVSEPDEPLFINRAGNRINTRNIFRICQRVLK |
| | QVLALLPENERFEFTPHKLRHTFLKKVTDKHGVHFAQELSGNVSIKEIFRYAKPSQDEMQETIEELFES |
| 1738 | WP_009408153.1 |
| | MVTFWLAGAGKSTLKLVVSSWGYEYPQMSGVRASDDPDSPMPGKENVPQAHKREAKARHARIIQLTELQL |
| | ATGLRASEARQITWADVTQDDHGVVWVTVRPEISKTKVGRTIPVLVPEVGKRLLARKGKDEELVIPQPNS |
| | GKPWDKAGVHKAVRDYYEGVAGTSEDLAFLKKIRSHAWRGALNTITAPHLRPDIRAAYFGHTEKVNARAY |
| | TDHVNVRPMMKAVEALTQRE |
| 1739 | WP_133288865.1 |
| | MATDGQDTEAGPGAGGANPPAQGRGSSTPPAPAAVPAVSGEPSEGTAAALPAAGREALDEALRYARAALS |
| | DNTRLAYAVDWQDFAAWCTAAGLAPLPAAAETVAAYLAALARTHAIATLRRRLVSIAKAHRVAGHTGFWA |
| | AHPVISETLRGITRTRGRPQRRAAALTTPDIRRLVATCGPDLAGLRDRALLLLGYAAALRRAELIAVEVE |
| | HLKFDAQGLRLHIPRSKGDQAGEGEELGIPRGQRRDTCPVRAIEAWMVASEAQYGPLFRKVNRWGGLEPG |
| | RLHPSSVRQILLRRAEQAGIAGTALEPISPHGLRAGFVTQAYKAGLRDEEIMQHSRHRDLRTMRRYVRRA |
| | KLLEDSPAKRLDL |
| 1740 | WP_011415080.1 |
| | MSDISPPSALPGSALAALETDLALALRAPNVAVDRELLAAYVEAAAPNSIRALRQDVEAFDLWCRRSDAR |
| | AFPATPGMVADWLKHRASEGAAPASLVRYKASIAKAHRLLDLDDPTKHEICRLAIAAHRRNVGSRQKQAR |
| | PLRFRGAVKDPVQATPRGIHIRAILGACDNTPTGLRNRALLSVAYDTGLRASELVAVAAEDIVEALDPDA |
| | RLLRIGRHKGDQEGEGSTAYLSPRSVQALQAWLHAADISEGPVFRRVIVRRYADRPARRRIDPNTISGRA |
| | IWDPRKFAAKAAVAARTEYNVGEKALHPGSVTPIVRGMIASAIDAGAFGDLDKEQARKLVAGFSAHSTRV |
| | GLNQDLFAIGETLAGIMDALRWKSPKMPLAYNRNLAAEAGAAGRLLSKLD |
| 1741 | YP_239821.1 |
| | MKTRCYDGKKWQYEFKHEGKRYRKKGFRTKREANSAGLDKLNELRQGFNFENNLTFEDYFKNWIETYKEN |
| | IVSENTFRHYRFTLKHIQRHKIGKVEISKINKQMYQKFINDFSENRAKETIRKTNGAIKSALEDAVYDGL |
| | IAKNPTYKITFKAGKTTKSEEEKYISLEEYKALKEYLKDKSSKSALALYIMICTGCRVSGVRSMKLEYIN |
| | EFRSELYIDEHKTDSSPRYVAVGKNDLRHIINVIKNSTISYDGYIFKDSGKLISINAINKTLKKACESLG |
| | INYITSHALRHTHCSYLLAKDISIYYISKRLGHKNISITTSIYSHLLEEKFSEEEQKTKNILDAM |
| 1742 | WP_018621639.1 |
| | METGESLPAVYSQITTDGQRLPGLNEQQQKAREFYESGLYGAPNSKKAYQSDVKQYLAWYHHKGYEALPS |
| | TSQALAEYMTELSTDKGYFTLQRRLASIAKYHRIHNLPSPTIHEQFKLFMKGVRREKTIRQKQAMAFTLD |
| | EFRQAVDSQPLTPTGLRNRLILLLGFTGAFRRQELVDINVENLECRSDGILITINHSKTNQDGVEEAKFV |
| | AKAKQEAYCPLRTLQQWLTLIGRAEGPLFVRIRKGERPTLDRLSDDYVNLLTKAAFGQYYSAHSMRASFV |
| | TISKDAGVDNRKIQNQTKHKTTOMIDRYDRRRDVIYQNASTELDL |
| 1743 | WP_026242320.1 |
| | MQNPPANMPEIADDHRDGDLPDSVDLVTETGASASPASARVEALVATATAYANAASSENTRSAYAKDFSH |
| | FTAWCRREGFEPLPPSSQVIGLYIGACASGSVVDTAAGKPKRTAPALSVATIERRLSGLAWNFTQRGMPM |
| | DRSDRHIATVLAGIRRKHGTPPRQKEAVLGEDIRAMINTLGHDLRGLRDRAILLLGFAGGLRRSEIVGLD |
| | IARDDKSDGHGWIEIFPDQGVLVTLRGKTGWRQVEVGRGASSETCPVAALESWIRFGRIARGPLFRRIFK |
| | DNKTVDVERLSDKHVARLVKQTALAAGVRSDLPEGERALLFSGHSLRAGLASSADIEERYVQKQLGHASA |
| | EMTRKYQRRRDRFRTNLTKASGL |
| 1744 | AVC45611.1 |
| | MPEIADDHRDGDLPDSVDLVTETGASASPASARVEALVATATAYANAASSENTRSAYAKDFSHFTAWCRR |
| | EGFEPLPPSSQVIGLYIGACASGSVVDTAAGKPKRTAPALSVATIERRLSGLAWNFTQRGMPMDRSDRHI |
| | ATVLAGIRRKHGTPPRQKEAVLGEDIRAMINTLGHDLRGLRDRAILLLGFAGGLRRSEIVGLDIARDDKS |
| | DGHGWIEIFPDQGVLVTLRGKTGWRQVEVGRGASSETCPVAALESWIRFGRIARGPLFRRIFKDNKTVDV |
| | ERLSDKHVARLVKQTALAAGVRSDLPEGERALLFSGHSLRAGLASSADIEERYVQKQLGHASAEMTRKYQ |
| | RRRDRFRTNLTKASGL |
| 1745 | WP_015494605.1 |
| | MLSHLVPLSRTGVKYPNVRQQGRSSIDPLEEKKTRRIEPTVADLASDWLDVHASGLKSEQAIRSLIGGDL |
| | VKAIGRMKVTDVRRRDVIEAVEAKATTAPRQAALMLSYARMLLDYATDRDIVRANPVAGLKPASIKVAGK |
| | RDPLKPVVRLRVLDAEEIKSMWVNVESCGLHLLTGLALKLVLVTGQRPGEVAGMHENEISGRLWTIPASR |
| | RGKTSTTQTVYLTDTALNIITVAKAELERLQGRRKGALSGYIFEAKGGSSITNSALPRGVQRSHEALGVK |
| | DNETWGHWTPHDLRRTMRTGLSACKIAPHIAELTIGHTKRGIVATYDQHTFDSERRDAMMAWELRLMTIV |
| | AGNNPDAIVDNVLKLEAKA |
| 1746 | WP_005610302.1 |
| | MPPKSHSQLNNEEPSTSEFSQDLSENTRKAYATDWALYMQWCRMQGIPPLSATPDHIARYLTEISASSGL |
| | SSASVRRRLAGLVWNYHQRGFRLDRNSPLIADALSEITTNEQCATVIKDAITPQEIRAMVATLPFDLRGL |
| | RDRAILLTGYIGGLGRSDLIGLDLHQYDTEGGTGWIELHSEGILLTIRSKTGWRKVQIACGNTDLTCPVY |
| | TLTKWLHFAKIRSGPAFVRTSRDDKRALSTRLNDRHIPRLVKSTILKAGIRAELPEKERLALFSGHSLKK |
| | GLSVSVDQSSGNQIEVNLTKAAGF |
| 1747 | WP_093220183.1 |
| | MTELDRYLQAATRDNTRRSYRAAIEHFEVTWGGFLPATADSVARYLVAHAGVLSINTLKLRLSALAQWHN |
| | SQGFADPTKAPVVRKVFKGIRALHPAQEKQAEPLQLQHLEQVVGRLEQEIQAAKAQADRPGLLRARRDLA |
| | LILLGFWRGFRSDELCRLQIEHVQAVAGSGITLYLPRSKSDRENLGKTFQTPALQRLCPVQAYIDWITEA |
| | ALVRGPVFRGIDRWGHLGEEGLHANSVIPLLRQALGRAGIAAEQYTSHSLRRGFATWAHQSGWDMKSLMG |
| | YVGWKDMKSAMRYIDASPFAGMALSAEKPVAAQIPNSSINTVG |
| 1748 | WP_065540814.1 |
| | MRRDIITRAMIEEFQNYLWEHEKAKLTIQKYISEIENLKEFLQGQPIGKSRLLEYRGQLQERYKARTVNA |
| | KLSAINAYLVFSGMEACKVKLLKIQHCSFIEENKELSEAEYRRLLSSAGKLKNKRMYYLLLTFGGTGIRV |
| | SELPFITVEAVRTGRADINLKGKNRTVILPKKLTDKLSRYAKEQGIHTGAVFCTSSGKKLDRSNICHDMK |
| | KLCKEANVDVHKVSPHNFRHLFARCFYAVHKNLAHLADILGHSSVETTRIYVQTSIREHERIISKMKLVV |
| 1749 | WP_044543906.1 |
| | MTPEPRALNDDESRYPGWFLAFMADRAVRKPSPHTLKAYRQDFIAIATQLAAAPDRVAYLTPDAITRDAM |
| | QAAFAAYADTHEAASIRRCWSNWNTLCDFLYTDDLITANPMPLIGRPKVPKSLPKGLGAETVSGLLEAIE |
| | ADSGSQRRSNWPERDAALVLTAVLAGLRADELLHADVGDIRATTEGGGVIQVRGKGNKDRRIPVEQGLIK |
| | VLECYLDSRAVRFPADTKRRGPSAGIGAWPATAALFVGSDGHRITRGVLQYRVLRAFKNAGLNGQRAAGA |
| | LVHALRHTFATELANSDVNVYMLMKLLGHESMVTSQRYVDGAGTQNRAAAAQNPLYGLIKQSREP |
| 1750 | WP_034396620.1 |
| | MDSSKPSPFPVPVIFDANRLQIQDVLESALAPKAHEAIKELMHEGESSNTRSSYQSAMRYWAAWHLLRLG |
| | QPMQLPLRASTVLQFIIDHAQRQSGAGLLHEMPPEIDEALVAAGYKGKKGAPSHSTLVHRMAVMSKAHQV |
| | HAMPNPCQDGAVKELMSRTRKAYARRGELPQKKEALTRDVLEELLASCDDSLRGLRDRALLLFAWASGGR |
| | RRSEVAGADMRFLRTVANGEFIYTLSHSKTNQSGTDAPENHKPVTGRAAIALKAWLDGARITEGPIFRRI |
| | RKGGHVAEPLTPAAVRNIVKERCALAGVEGDFSAHSLRSGFVTEAGRRNLPLADTMALTGHHSVNTVLGY |
| | FRADSALNNQAARMLDED |
| 1751 | WP_048444547.1 |
| | MDLDRADPTPESPLEALALPVPPAAGLPPTDEILIERLEGHARAAHGAFADNTVRAFAADSRIFAAWCRE |
| | AGRTMLPATPETIAAFIDAQAETKSRATVERYRSSIAALHRAAGLANPCADEIVRLAVKRMNRAKGRRQK |
| | QAEPLNRTSIDRMIAVKAVERLHRRVSEGEHGAPLIALRNIALVAVAYDTLLRRSELVSLSIEDLERGAD |
| | GSGTVLVRRSKADQEGEGAIKYLAPDTMAHVEAWLAAAGLESGPLFRPLTKSGKVGARALGDRDVARIYR |
| | ALASAAGLKIPRLPSGHSTRVGATQDMFAAGFELLEVMQAGSWKTPAMPARYGERLRAQRGAARKLATLQ |
| | NRA |
| 1752 | WP_003499734.1 |
| | MTTYVITAEMEILFGEYLEQDEKSKNTIEKYRRDLRKFVEYIDGEEVTKELVIGFKEYLVEHYAVNSVNS |
| | IIASLNRFMQFAGWQEFRVKQLKKQRQVYCPEEKELTKQEYFELIRTAKREGKEKIGLIIQTIGSTGIRI |
| | SELPSITVQAVKNGVAQVDCKGKNRQVLLPRKLLVKLMHYIRKEHIQCGPIFITKQGNPLDRSNIWKEMK |
| | KICRLAGVNEKKVFPHNLRHLFAYSFYQMEKDIAKLADLLGHSNINTTRIYIVSSGVEHRKQIEKMNLLL |
| 1753 | WP_001066953.1 |
| | MNNVIPLQNSPERVSLLPIAPGVDFATALSLRRMATSTGATPAYLLAPEVSALLFYMPDQRHHMLFATLW |
| | NTGMRIGEARMLTPESFDLNGVRPFVRILSEKVRARRGRPPKDEVRLVPLTDISYVRQMESWMITTRPRR |
| | REPLWAVTDETMRNWLKQAVRRAEADGVHFSIPVTPHTFRHSYIMHMLYHRQPRKVIQALAGHRDPRSME |
| | VYTRVFALDMAATLAVPFTGDGRDAAEILRTLPPLR |
| 1754 | WP_001066942.1 |
| | MNNVIPLQNSPERVSLLPIAPGVDFATALSLRRMATSTGATPAYLLAPEVSALLFYMPDQRHHMLFATLW |
| | NTGMRIGEARMLTPESFDLDGVRPFVRILSEKVRARRGRPPKDEVRLVPLTDISYVRQMESWMITTRPRR |
| | REPLWAVTDETMRNWLKQAVRRAEADGVHFSIPVTPHTFRHSYIMHMLYHRQPRKVIQALAGHRDPRSME |
| | VYTRVFALDMAATLAVPFTGDGRDAAEILRTLPPLR |
| 1755 | WP_015469749.1 |
| | MDHSLWIDFFDDLQNVRGRSKNTVMAYRRDLELYKKFTEKSKRVIEFFDFMKKEGLSTRSQARVISSVRT |
| | YLKFCESKGMKCPDLRELRPPKVKTGLPKAVSVEEFEKLFRACAVEGEARTARNQLTLLFLYGLGCRVSE |
| | LIGLSLHDFSPTERWVKVLGKGSKERLIPLTDTLYNALEEYLKNHRSELMMDNKSNAALLLNDRGHRPSR |
| | VDIWRWLASWSAKAGFDEPVHPHRFRHGCATALLEGGADLRSIQVMLGHASIQTTQVYTAVTTNTATKAI |
| | DEHHPLSKIKDFSG |
| 1756 | WP_012187369.1 |
| | MEETPEIQPPDAEKSTSAPSDSNNQAQRDERDQVSTDPIALPAHVAGSGTLDRLVDTARDYARASTAENT |
| | NKAYAADWKHFARWCRLKGTDPLPPSPEMIGLYLTDLAAPAKGTPALSVSTIERRLSGLAWNYAQRGFSL |
| | DRKDRHIANVLAGIKRRHARPPVQKEAILPEDILAMVATLPFDLRGLRDRAILLIGFAGGLRRSEVVSLD |
| | VSKDDTPDSSGWIDVFEDGAVLTLNAKTGWREVEIGRGSSEQTCPVHALEQWLHFAKIDFGPVFTRTSRD |
| | GKRAMDERLNDKHVARLIKKTVLKSGIRAELPENERLALFSGHSLRAGLASSAEVDERFVQKQLGHASAE |
| | MTRRYQRRRDRFRVNLTKAAGL |
| 1757 | WP_056515134.1 |
| | MATFRQRKDSWRVEVSVKGVRDSGTFDTKTQAKAWAAKRETELRDQANGKLPSFTLQNAIDRYVREVSIN |
| | KKAHYKEIARIKVFCKSYPNLCKKQISKITTDDLVQWRDSRLKEVQGATVRREATILSGIFTVAKKEWKW |
| | IYESPLTDLSMPPIAKSRDRRVTQDEIDRLCLAAMWDESTPTTSTQQTIIAFLLALETAMRAGEILTLTW |
| | DRVFLEERYVALDETKNGTKRNVPLSKRAVELIVHLKPLDKTMVFTCKRSSFSALWIDLKKKCKIEDLHF |
| | HDSRHEACTRLARKLDVLDLARMIGHKDLKSLMVYYNATATEIAHRLD |
| 1758 | WP_051472036.1 |
| | MADAETTPDLDVEVVDALPTVVPSHLPGELQDLLDAAREYADAATAPNTVKAYQSDWRGFTAWCAQHRLQ |
| | PLPADSMTVALYLTAMAKNGRKVTTIRRHTAAIARAHRDNGLPNPMWDPTAALVLEGIARTHRSAPKKKV |
| | ALLRDPMVQLIDRIETDTPAGLRDRALLLLGFALGLRRSELVQILIEDLSPNADGLTIRLATSKTDQTGH |
| | GHEFLLPYAEPGRPCPVRAIRAWLDHTGLTHGPLIRRLHRNGIPGEALSPQSVALIVKRRAKAAGLNPAD |
| | FAGHSLRAGFATQASRDGHRTEQITDVTRHRDRRTLDGYVRAGKGAEDVARVL |
| 1759 | WP_016391764.1 |
| | MGAQATRLSDLKVKAAKPKEKDYTLTDGNGLQMRVRINGSKLWNFNYIHPVTKKRINMGLGTFPEVSLAQ |
| | ARKRTVEAREIVAQGLDPKEQRDAERQAKKAATEHTFENVTTAWFELKKDSVTPAYAEDIWRSLTLHIFP |
| | DLGTTPISAINAPKVIDLLRPLETKGSLETVKRLTQRLNEVMTYGVNSGLIHANPLSGIRSVFKKPKKKN |
| | MAALPPDELKELMVAIANASIKRTTRCLIEWQLHTMTRPAEAATTRWADIDIEKKVWTIPAERMKKRRIH |
| | IVPLTDQALDLLEAIKPYSGHREYVFPADRNPRTHCNSQTANMALKRMGFEGRLVSHGMRSMASTILNEH |
| | AWDPELIEVALAHVDKDEVRSAYNRADYIERRRPMMKWWSEHIQEAATGNLSMSAVQNNRDRKVVSIR |
| 1760 | WP_052959163.1 |
| | MQLNTVYSYSVTEAQVYNSFDIDRASENCSAETVEYLKQCQALTGKQIISLRGDSSFDEAFMLFTRLSLL |
| | VTRRRPELGVHCILIHAMPVIGGMKVEDMNRLAINRLINALVLDGKLVQSRRVFSVIKQFLGWCEFQGI1 |
| | ESSPLATMSLNKVAGGAKPKPRERVLSDDELEKFWHMWDFAEVSESTRWAARFILCSARRPDEVLRARRD |
| | EFDLHKDVWNQGERNKSGRDHSLPISPIMRMCIDKMIDAAGDSEWLVPSPKTTAKPASKVMVAQASRRIM |
| | AKKYLSDALPEPFEIRDLRRTARSSLSRLNVDQDVARKIMNHSLEGIDRVYNRHDYMDRMIEAMLAYSDF |
| | LMEKCKINQ |
| 1761 | AGC72343.1 |
| | MTELATITTNTIQPIINLVCNAVTSDHTKRAYSRALTDFIAWHSSTGQQGFGKATVQAHVTALRDAGVSA |
| | SSINQRLTAIRKLAVELADNEVIDHSAAQAVGRVEGVRKEGKRLGNWLTKEQAQQLLTLQPIATVKGLRD |
| | RAILAVLLGCGLRREECTGLAVGNIQQREGRWVIVDLVGKRSKTRSVPMPAWCKYAIDAYLLAAAVTDGV |
| | LFRSVRRGDHITGQGMTAQAIFDVVKDYAKEIGVDVRPHDLRRTFAKLAHKGNAPIEQIQLSLGHSSVQT |
| | TERYIGVQQDLSSAPCDALGLRI |
| 1762 | WP_117316704.1 |
| | MSYLATSPIDGFVSVYKEAVNKYFKDIFTKLPHNTQRAYVSDFNEFAIFCEQEGLTGFNDSMQNNEDCIK |
| | RYVEVLCHSPLAYRTIKRRLSALSKFLGIAQLPNPIVNSVYLKDFVKLSLSQNEKYQLSSHQAVPLTVDI |
| | LEKINNNVIPDTLLEMRDLAIINLMFDGLLRADEVVRVRTEHINKRNNALLVLTSKSDQTGKGSYRFISN |
| | STVAMIDEYINEANYNKALQQERDSSDPRRINHGILFRRVSNRGHALLPYDEQLKGKHSPILEYSSIYRV |
| | WKRIADMAKVKENITPHSGRVGGAVSLAENGATLPEMQLAGGWHSPEMPGHYGQQAAVGKGGMAKLAQLK |
| | GR |
| 1763 | WP_020744756.1 |
| | MNTKPPKMATDLALRNEESNQIAHRESESLRHYLQAATSDNTRKAYRSAIRQFEKWGGRLPTDRDTVVRY |
| | LLARAESLNARTLDLHLTAISQWHHYQGVTDPVRDPLVRKTMEGIRRTHGQPKRKAKALRLEHIAQMVDY |
| | LRCLPDSKKKHRDIALVLTGFFGAFRRSELVAIQISDLNWEPEGLIIRLPRSKTDQQAAGLARALPFGTP |
| | GCCPATAIKAWMDSAEIDSGPLFRPANRWDQVTPRPLNPGAINDLLKTLGKACQFDFVPELSSHSFRRGL |
| | STSAARERVDFELIKKQGGWKSDATVWEYIEEGQQLSNNASLILMEKLVTLIDPV |
| 1764 | WP_017437096.1 |
| | MENSKIALQLFIEYLQIEKNYSQYTIVCYRQDIEQFFEFMNEQGIQHLHEVTYSDVRLYLTKLYGQKQSS |
| | RSISRKMSSLRSFYKFLLRERKVKENPFALAALPKKEQKIPNFLYPQELECLFHVNDVNTAIGQRNQALL |
| | ELLYATGVRISECCHIQLSDIDFSVSTILIHGKGSKQRYVPFGRFAKEALERYIRQGRRELLENAKTEHA |
| | YLFVNARGNPLTPRGARYILDEIVKKAALTQHISPHVLRHTFATHLLNEGADMRTVQELLGHAHLSSTQV |
| | YTHVTKDRLRHIYLHTHPRA |
| 1765 | WP_054292066.1 |
| | MRIVGGYRYQDHVLLLLDAQDAFFLARKPRKDSPHTTAAYRRDLSGITTLLAGTTGRPVEHLTIQDLTVQ |
| | ALRTAFGDFADGHAKSSVARAWSTWNQFLNFCVADGMLDGNPMGAVVRPKAPLPSPKPLRGEDTPERLIA |
| | AAAAGARKARDPWPERDVLVIALGLVAGLRSAEVRALQRCSIVGRPGEQRLHVQGKANRERSIPIEAPLE |
| | RVIGAYLASCEVRFPHQRFGPVSALLLDYQGKPIGRGALDYLVKTSYQWAGIRDQVPTGANLHALRHTFA |
| | TRLAEDGANAAEIMALLGHANLNTSQNYIEATGRERRAAAAGNRTYRALSGLEPGTTSPDS |
| 1766 | WP_012862144.1 |
| | MENKTVAMEFLDYLRYEKGSSENTLSSYKRDLNLFFSEVPKNFQSIEDEEVIEYVDKLSKTVKRNTVLRK |
| | IASIRAFYKFCYINKYITDNPTESLKNLKREFKLPEVLKLSEIKDIIDAIPNTPEGVRDKIIIKILVATG |
| | ARISEVLTLDIKDVENQDYEFIRVLGKGSKYRLIPIYSQLEEEIKAYIENDRKILVLERKEKESENKNKK |
| | KGHELEYKLFLGTRRENFWKRLKKYAKNAKIEKNVYPHIFRHSVATMLINNGADIRIVQEILGHVNISTT |
| | EIYTHVGKRELKEIYNKVKIGDEE |
| 1767 | WP_022684352.1 |
| | MGTDTAREERESVVAAAETALTPIAPFGDLPWAIVQMRAVEPIVVNEALIAAYQAASSPHSVRALRSDIE |
| | AFDAWCRRTQRIALPATPEMVADYLDARAGEGAKPASLGRYKASIAKVHQLLELKDPTQAPLVKLRLAAI |
| | RRRTGTAQKQARPLRFKGPVKDVERDQARGLNIRALLEACGDDLPGLRDRALLSAAYDTGLRASELVAVA |
| | VEHIVDALDPEARLLEIPRSKADQEGEGATAFISPRSVRAIAAWRAASGIAAGPLFRRVQVRRYKARLAD |
| | PGRPIASISGREAWDLRKTLPKRAMAARVEYDVGEAALHPGSIGPIWRRIIQRAFDRGALGDLTADDLVR |
| | LLKGISAHSTRVGLNQDLFASGEGLTGIMDALRWKSPRMPLAYNRNLAAEQGAAGRLMAKLG |
| 1768 | WP_076797908.1 |
| | MASIWERKKADGSTSFTVRWRNPKTRKQEGITFSTAAEAQTLKRLLDANDQSFEIAQHAMVKNQTKAPTV |
| | AAVIQEHIDLLVRPSVGTVHTYQTMLKLHIADVIGHIPVDKLDYRHVTHWIKSMQAKGRSPKTIKNNHAL |
| | IYGAMETAVMLRYRKDNPCQRVQLPSSEKAEDEARFLTHAEFGLILECMGERYKAFTEFLVMTGLRFGEA |
| | TAVTVGDIDLMSKPATMRINKAWKRGTNSEFYIGATKTGAGKRTVSLNPQLVEILVPLVASRPGSDLLFT |
| | TPKGERIIHKLYWHHYWVPAVAAAQARGLKKSPRIHDLRHTHASWLIQDGVSLFTVSRRLGHASTRTTEQ |
| | VYGHLMPQALQDAADAVERSAVIWRS |
| 1769 | WP_097452609.1 |
| | MGELVIPGGSGGFLRDIGTEYQEAAKNFMQFMNDQGAYAPNTLRDLRLVFHSWARWCNSRQRPWFPITPE |
| | MAREYLLQLHEADLASTTIDKHYAMLNMLLSQCGLPPLSDDKSVSLAMRRIRREAATEKGERTGQAIPLR |
| | WDDLKLLDVLLSRSERLVDLRNRAFLFVAYNTLMRMSEISRIRVGDLDQTGDTVTLHISHTKTITTAAGL |
| | DKVLSRCTTAVLNDWLEVSGLREHPDAVLFPPIHRSNKARITTTPLTAPAMEKIFSDAWGLLNKRDATPN |
| | KGRYRTWTGHSARVGAAIDMAEKQVSMVEIMQEGTWKKPETLMRYLRRSGASVGANSRLMDS |
| 1770 | WP_016262425.1 |
| | MGELVISGGSGGFLRDIGTEYQEAAKNFMQFMNDQGAYAPNTLRDLRLVFHSWARWCNSRQHPWFPITPE |
| | MAREYLLQLHEADLASTTIDKHYAMLNMLLSQCGLPPLSDDKSVSLAMRRIRREAATEKGERTGQAIPLR |
| | WDDLKLLDVLLSRSERLVDLRNRAFLFVAYNTLMRMSEISRIRVGDLDQTGDTVTLHISHTKTITTAAGL |
| | DKVLSRYTTAVLNDWLEVSGLREHPDAVLFPPIHRSNKARITTTPLTAPAMEKIFSDAWGLLNKRDATPN |
| | KGRYRTWTGHSARVGAAIDMAEKQVSMVEIMQEGTWKKPETLMRYLRRSGASVGANSRLMDS |
| 1771 | WP_077543356.1 |
| | MGELVISGGSGGFLRDIGTEYQEAAKNFMQFMNDQGAYAPNTLRDLRLVFHSWARWCNSRQRPWFPITPE |
| | MAREYLLQLHEADLASTTIDKHYAMLNMLLSQCGLPPLSDDKSVSLAMRRIRREAATEKGERTGQAIPLR |
| | WDDLKLLDVLLSRSERLVDLRNRAFLFVAYNTLMRMSEISRIRVGDLVQTGDTVTLHISHTKTITTAAGL |
| | DKVLSRYTTAVLNDWLEVSGLREHPDAVLFPPIHRSNKARITTTPLTAPAMEKIFSDAWGLLNKRDATPN |
| | KGRYRTWTGHSARVGAAIDMAEKQVSMVEIMQEGTWKKPETLMRYLRRSGASVGANSRLMDS |
| 1772 | WP_032152854.1 |
| | MGELVISGGSGGFLRDIGTEYQEAAKNFMQFMNDQGAYAPNTLRDLRLVFHSWARWCNSRQRPWFPITPE |
| | MAREYLLQLHEADLASTTIDKHYAMLNMLLSQCGLPPLSDDKSVSLAMRRIRREAATEKGERTGQAIPLR |
| | WDDLKLLDVLLSRSERLVDLRNRAFLFVAYNTLMRMSEISRIRVGDLDQRGDTVTLHISHTKTITTAAGL |
| | DKVLSRYTTAVLNDWLEVSGLREHPDAVLFPPIHRSNKARITTTPLTAPAMEKIFSDAWGLLNKRDATPN |
| | KGRYRTWTGHSARVGAAIDMAEKQVSMVEIMQEGTWKKPETLMRYLRRSGASVGANSRLMDS |
| 1773 | WP_013160348.1 |
| | MAGKRAFGQIDRLPSGNYRARYVGPDLVLHKAPHTFTNKSHAERWLLDEQDLISRDVWEAPEVRTAKPRA |
| | LTVGEWISKVIERRANRTRRPLAQTTIDLYRKDYRLRISETLCAVRLADLTPAMVATWWHALPDTPTQNA |
| | RAYALLRSAMSDAMEDELIERDPCRLKEAGKPTPAHTGEAITVPELFTYLEAVPESRRLPLMIAALCGLR |
| | SGEVRGLRRRDVDLKAGMLHVEQAVSRVRADNHRWEWRIAPPKTAAGVRTVALPSPVTDALRTWLKEAPV |
| | NGWDGLLFPATDGHSPMPGTVLRDAHVKGREAIGRQTLTIHDLRRTAATLAAQGGATTKELMRLLGHTTV |
| | SVAMLYQVADEERDRARAQRLTQQLREGQAGQ |
| 1774 | EHJ58476.1 |
| | MTALILVPMTTDPPEPLALPSPAAAPPDPSVAQVVEDVRDLVGAGIRLDQELVAAAVRGWSNNTRRAFCS |
| | DLKVWGDWCRRHGIAPVRATQSDVAAYIRALSGIDPSAEKVRAMATIERYVSYIGRAYRMAGLADPTAGE |
| | LVTLEKKAARKKRGVRQRQARAIRFKGDIADFDSPPSGVCLAHLIKAVRRDVMGLRDEALLRVAYDVGAR |
| | RSELVAIDVDHIHGPDAGGAGALFVPTSKTDQEGEGAWAYLSPATMKAIARWREAAHIDKGPLFRRIETH |
| | FDGSIAAIGTKRLHPNSITLLYKRLVQRAFDKKLLGPMSEAEVARWVAAVSSHSLRVGVAQDNFAARESL |
| | PAIMQAYRWRDPKTVLRYGAQLAAKSGASARMAVRVGE |
| 1775 | WP_039858563.1 |
| | MTTDPPEPLALPSPAAAPPDPSVAQVVEDVRDLVGAGIRLDQELVAAAVRGWSNNTRRAFCSDLKVWGDW |
| | CRRHGIAPVRATQSDVAAYIRALSGIDPSAEKVRAMATIERYVSYIGRAYRMAGLADPTAGELVTLEKKA |
| | ARKKRGVRQRQARAIRFKGDIADFDSPPSGVCLAHLIKAVRRDVMGLRDEALLRVAYDVGARRSELVAID |
| | VDHIHGPDAGGAGALFVPTSKTDQEGEGAWAYLSPATMKAIARWREAAHIDKGPLFRRIETHFDGSIAAI |
| | GTKRLHPNSITLLYKRLVQRAFDKKLLGPMSEAEVARWVAAVSSHSLRVGVAQDNFAARESLPAIMQAYR |
| | WRDPKTVLRYGAQLAAKSGASARMAVRVGE |
| 1776 | WP-053559035.1 |
| | MPTVVQLPAGKVLTVRAAADAFLDSLRNPNTVRSYGVGVGKTAERIGEARPLGSVADDEIGEALELLWGT |
| | AAVNTWNARRAAVLSWLGWCAEYGYDSPSVPAWTKRLAVPDSETPARSKMAVDRLIARREVHLREKTLWR |
| | MLYETCARAEEILGVNIEDLDLAARRCPVKAKGARSKARRRGQAREDFVLETVYWDAGTARLLPRLLKGR |
| | TRGPVFVTHRRPGPGKVVSPRDVCPDTGLARLSYGQARALLDERTAVHGPGTGWDLHEYRHSGLTELGVQ |
| | GASLLMLMAKSRHKKPENVRRYFHPSPEAISELTSLLAPGDGRR |
| 1777 | SEC15746.1 |
| | MSSDNEQNPRMPGMIQPAAPERPSNRVAASGNDGTSALKSGKPAGDHSDLPDLIDVVLAMDEEPPSPTRR |
| | SPALPSNVDPLVETARSYARAARSEATQRAYAADWRHFASWCRRSGFQPLPADPQVVGLYLTACASANPR |
| | PTVSTLERRLSGLSQGYSQRGDRLDRKDPHIAEVFAGIRRKHGRPPAQKEALLAEDILAMLETLPHDLRG |
| | LRDRAILLVGFAGGLRRSEIVGLDAGVDQTEDGNGWAELFDKGILITLRGKTGWREVEIGRGSADRSCPV |
| | EALRTWTRLARIAHGPLFRRIRGQKTVEDSRLNDRHIARLVKRTAFDAGLRPDLPEKERERLFSGHSLRA |
| | GLASSAEVDERFVQKQLGHTSAEMTRRYQRRRDRFRVNLTRASGL |
| 1778 | WP_090330126.1 |
| | MPGMIQPAAPERPSNRVAASGNDGTSALKSGKPAGDHSDLPDLIDVVLAMDEEPPSPTRRSPALPSNVDP |
| | LVETARSYARAARSEATQRAYAADWRHFASWCRRSGFQPLPADPQVVGLYLTACASANPRPTVSTLERRL |
| | SGLSQGYSQRGDRLDRKDPHIAEVFAGIRRKHGRPPAQKEALLAEDILAMLETLPHDLRGLRDRAILLVG |
| | FAGGLRRSEIVGLDAGVDQTEDGNGWAELFDKGILITLRGKTGWREVEIGRGSADRSCPVEALRTWTRLA |
| | RIAHGPLFRRIRGQKTVEDSRLNDRHIARLVKRTAFDAGLRPDLPEKERERLFSGHSLRAGLASSAEVDE |
| | RFVQKQLGHTSAEMTRRYQRRRDRFRVNLTRASGL |
| 1779 | WP_025031421.1 |
| | MPGMIQPAAPERPSNRVAASGNDGASALKSGKPAGDHSDLPDIIDVVLAMDEETPSPTRRSHALLSNVDP |
| | LVETARSYARAARSEATQRAYAADWRHFASWCRRSGFRPLPADPQVVGLYLSACASASPRPTVSTLERRL |
| | SGLSQGYSQRGDRLDRKDPHIAEVFAGIRRKHGRPPAQKEALLAEDILAMLETLPHDLRGLRDRAILLVG |
| | FAGGLRRSEIVGLDAGVDQTEDGNGWAELFDKGILITLRGKTGWREVEIGRGSADRSCPVEALRTWIRLA |
| | RIAHGPLFRRIRGQKTVEDSRLNDRHIARLVKRTAFDAGLRPDLPEKERERLFSGHSLRAGLASSAEVDE |
| | RFVQKQLGHTSAEMTHRYQRRRDRFRVNLTRASGL |
| 1780 | WP_070174536.1 |
| | MQSPFINAVYEFMMLKRYAKRTIQSYLVWIADFIRFHKYQHPKTMGDQEVSLYLTHLSVKRNLSASTQAS |
| | ALNALVFLYNKYLLQPLSKEMEFVNSGRKPKLPTVLTISEVQQLLTNIPERQSLPVSMLYGSGLRLMECV |
| | RLRVKDIDFDYRCVRIWQGKGGKHRVVTLSDTLIAPLKTQKEKVRHLLERDCSNPEFAGVWMPHQLAKKY |
| | KSANKSLEWQYLFPASKTSIDPESALRRRHHIDEKQLQRAVRQTAQEIGLQKSVTPHTLRHSFATHLLLK |
| | GADIRTVQEQLGHSDVRTTQIYTHILQRGGSAVVSPLESI |
| 1781 | WP_039328773.1 |
| | MTELTPFSGPLIPGDADMADRLREFVQDREAFSDNTWRQLLSVMRTGSRWADEHGRRFLPMTPADLRDYL |
| | LWLQATGRASSTITTHAALISMLHRNAGLVPPNRSPVVFRAVKRIHRTAVISGERAGQAVPFRIGDLLTL |
| | DKSWCFSDRLQQLRDLAFLHVAYATLLRVSELGRLRVRDISRAPDGRIVLDVAWTKTIVMTGGLIKGLGD |
| | LSSQRLTAWLTVSGLIAEPDAFIFGPVHRTNRALPATEKPLTTRALEDIFARAWQEAGPGQDAKPNKNRY |
| | RGWSGHSTRVGAAIDMATKKYSTAQIMQEGTWKKAETVMRYIRHVDAHAGAMVEFMDKHYSGN |
| 1782 | WP_105080092.1 |
| | MDDFRPHLPVSDAQLPGQADVVAQLREFVQDREAFSDNTWRQLLSVMRICAGWAKEYGRTFLPMTPECLR |
| | DYLMWLQANGRASSTIGTHLALISMLHRNAGLTPPHASPLVFRAMKKISRTAVVSGERTGQAIPFRLTDL |
| | LTLDARWSGSDSLQHQRDLAFLHVGYSTLLRVSELGRLRVRDVSHASDGRIVLDVGWTKTIVMTGGLIKG |
| | LGALSTQRLREWLNASGLINEPDAFIFSPVHRTNRAKINTDRPLSTRSMEDIFARAWHEAGPAADVKPNK |
| | NRYRRWSGHSARVGAAIDMATNKYSTAQIMQEGTWKKAETVMRYIRHVDAHSGAMVDFMDAHVGR |
| 1783 | WP_042596186.1 |
| | MSNREKQVYQARVERHKEKMPWYIIEYIEEKTNLSPTTLYGYLIEYEIFLQWLISSHLATSDGEVVTKIH |
| | EVPIETLEHLPLKQVKRFKSYLERQGNKTKAVIRTFSALKSLFNYLTSNTEDDNGECYFYRNVMAKMEIH |
| | KEKIDAAARAKEISEVIFHNNDDIKFMRFLSNEYEOMLQETAPGKLRFFKRDRERDIAILSLILGTGLRV |
| | SEVASLTISSINFRTRYIKVIRKGDKKSSILATQTALDDVQEYLKVRANRYKCPDDEDILFVTNYKGSYA |
| | QISVNAIQKLTEKYTRAYDEKKSPHKLRHTYATNHYNENKDLVLLANQMGHNSMETTSLYTNIDDTKRRA |
| | AIERLEQRQFEDTKEK |
| 1784 | WP_113233496.1 |
| | MPKPKALPSPADPVLARAEELDALDAILPFARRDQLAALLTDEDVATLKHLAKEGMGDNTLRALASDLGY |
| | LEAWCELSTGAPLPWPAPESLLLKFVAHHLWDPVERAEDPSHGMPDDVEMGLRAKGLLRADGPHAPDTVR |
| | RRLTSWSILTRWRGLTGSFTAPSLKSALRLAVRASARPRRRKSKKAVTGQILAKLLATCDGERLVDLRDR |
| | ALLLTAFASGGRRRSEVAGLRVEDLVDEEPVHADPRNAASPLLPCLTINLGRTKTMTAEDRVHVVLIGRP |
| | VEALKQWTEEARIDAGPVFRRIDQWGNIDRRALTPQSVNLILKTRCSQAGLDPELFSAHGLRSGYLTEAA |
| | NRGVPLQEAMQQSLHKSVAQAASYYNNAERKNGRAARLVI |
| 1785 | WP_110880404.1 |
| | MAKTTPSDAIHRRAEDLDALDSILPFDRRDQLAALLTDDDVATLKHLANEGMGDNTLRALTSDLGYLEAW |
| | CQLATGSPLPWPAPESLLLKFVAHHLWDPAKRAEDADHGMPADVEAGLRDSRLLRAKGPHAPDTVRRRLT |
| | SWSILTRWRGLTGTFNGPSLKSALRLAVRASARPRQRKSKKAVTADILAKLLQACAGDRLVDLRDQALLL |
| | TAFASGGRRRSEIAGLRVADLVDEEPVRADPNDANSPALPCLSIRLGRTKTTTSDDDEHVVLIGRPVVAI |
| | KHWLEQANVKDGPVFRRIDQWGNIDRRSLTPQSVNLILKTRCKQAGFDPALFSAHGLRSGYLTEAANRGI |
| | PLPEAMQQSLHKSVTQAARYYNDAERKQGRAARLMI |
| 1786 | WP_120019218.1 |
| | MPKNPARAGGRQSTELVARRAEALDALDSVLPFDRRDFLAGLLTDDDVATLRHLAKEGIGANSLRALASD |
| | LGYLEAWSLAATGFSLPWPAPEALLIKFVAHHLWDLAKRETDPAHGMPADVAATLKSQALLRTDGPHAPA |
| | TVRRRLSSWSTLTKWRGLRGKFNAPGLQSAIKLAVRASARPRGRKSKKAVTADILTALLKACAGDRLVDV |
| | RDRALLITAFASGGRRRSEMASLRFEQIVEEEPVPAEPKAPDSSDKLPCLSIRLGRTKTTQADSDAFVLL |
| | VGRPVLALKGWLERAGITEGAVFRGIDRWGNLEKRALTPQAVNLLLKRRIAEAGLDPQAFSAHGLRSGYL |
| | TETARRGIPLPEAMQQSQHRSVQQASNYYNDAERTLGRAARVI1 |
| 1787 | WP_069694292.1 |
| | MPSSQASPPKIDGMAVARINGEQPDIAEPVIEIGAAEPATLIPARLEALVETATGYAKAASSENTRAAYA |
| | KDWRHFSSWCRREGLEPLPPSSQVIGLYISACAAGEPKRGLPSLSVATIERRLSGLGWNFNQRGQPMDRA |
| | DRHISTVLAGIRRKHAKPPRQKEAVLGDDLLAMIATLGHDLRGLRDRAILLLGFAGGLRRSEIVGLDVVR |
| | DDNSDGAGWIEIYADKGVLVTLRGKTGWREVEVGRGSSDHTCPVVALESWVRFGRIARGPLFRRIFKDNK |
| | TVDVERLSDKHVARLVKQTVLEAGVRSDLPEGERALLFAGHSLRSGLASSAEIEERYVQKHLGHASAEMT |
| | RKYQRRRDRFRTNLTKASGL |
| 1788 | WP_092177345.1 |
| | MTSIALLSTETETRRALQLDALAGILPLERRDKLAHILTDDDVATLRHLAREGMGENSLRALASDLAYLE |
| | AWSLASTGSALPWPAPEALVLKFVAHHLWDPAQRESDPAHGMPAEVDEVLRAGDHLRSAGPHASSTVKRR |
| | LAHWATLHRWKGLESPLGTPAIRTSVRLAVRASAKPRRRKSKRAVTRDILDRLIATCQSDRLADTRDLAI |
| | LLVAFASGGRRRSEVARLRLEQLTLEADVPLDPDDPNSARLPCMAIALGRTKNSQADDDARVLLIGPPVE |
| | ALREWLERAAISKGAVFRAIDRWEAIDDRALTPQAINLILKRRCALAGLDPVAFSAHGLRSGYLTEAARN |
| | GVALPAAMRQSQHRSVQQAARYYNDADQALGKAARLAI |
| 1789 | WP_057193706.1 |
| | MTSTSLLSAETETRRALQLDALAGILPLERRDKLATILTDDDVATLRHLAKEGMGENSLRALASDLAYLE |
| | AWSFASTGSALPWPAPEALVLKFVAHHLWDPTQRESDPAHGMPAEVDAALRAGDHLRSEGPHAPSTVKRR |
| | LAHWATLHRWKGLESPLGTPAIRTSMRLAVRASAKPRRRKSKRAVTRDILDRLIATCQTDRLADTRDLAI |
| | LLVAFASGGRRRSEVARLRLEQLTLEADVPLDPDDPNSPRLPCMAIALGRTKTAQADDDARVLLIGPPVA |
| | ALREWIERAAIKTGAVFRAIDRWEAIDDRALTPQAINLILKRRCAMAGLEPIEFSAHGLRSGYLTEAART |
| | GVTLPAAMRQSQHRSVQQAARYYNDADQALGKAARLAI |
| 1790 | WP_133565315.1 |
| | MTTTALLSADSETRRALQLDALAAILPLERRDQLAKILTDDDVATLRHLAQEGLGENSLRALASDLAYLE |
| | AWSLASTGSALPWPAPEALVLKFVAHHLWDPAQRETDPAHGMPAEVDAVLRSGDHLRSDGPHAPSTVKRR |
| | LAHWATLHRWKGLMGPFAAPSLRTAMRLAVRASARPRRRKSQRAVTREILDRLLATCRSDRLSDTRDLAL |
| | LLTAFGSGGRRRSEIARLRVEQLSEEAPVPLDPEDLNSPRLPCLAITLGRTKTAMANDDARVLIVGPPVE |
| | ALREWLERANISKGAVFRAIDRWEGLSDRALTPQAVNLILKRRCAQAGLNPWEFSAHGLRSGYLTEAARN |
| | GVSLPAAMQQSQHRSVQQAASYYNEADRQLSKAARLAL |
| 1791 | KSV89580.1 |
| | MVSAVESPSPLPAHLEDLADRARGYVEAASSANTRKAYASDWKHFSAWCRRQNLAPLPPDPHVVGLYITA |
| | CASGTTERSVKANSVSTIERRLSAIAWNCTQRGQPLDRKDRAIATVMAGIRNRHAAPPRQKEAILPEDLI |
| | AMLETLERGTLRGLRDRAILLIGFAGGLRRSEITGLDLGRDQTEDGRGWIEILDKGLLLTLRGKTGWREV |
| | EIGRGSADTTCPLVAVETWIRFAKLAKGPLFRRVTGRGKDVGPDRLNDKAVARLVKSAALAAGLHGDLGE |
| | DERAARFSGHSLRAGLASSAEVDERHVQKQLGHASAEMTRKYQRRRDRFRVNLTKASGL |
| 1792 | WP_058323347.1 |
| | MAQIVAQNRKNHAKETSAPSDSTAHSGDDPALVSAVESPSPLPAHLEDLADRARGYVEAASSANTRKAYA |
| | SDWKHFSAWCRRQNLAPLPPDPHVVGLYITACASGTTERSVKANSVSTIERRLSAIAWNCTQRGQPLDRK |
| | DRAIATVMAGIRNRHAAPPRQKEAILPEDLIAMLETLERGTLRGLRDRAILLIGFAGGLRRSEITGLDLG |
| | RDQTEDGRGWIEILDKGLLLTLRGKTGWREVEIGRGSADTTCPLVAVETWIRFAKLAKGPLFRRVTGRGK |
| | DVGPDRLNDKAVARLVKSAALAAGLHGDLGEDERAARFSGHSLRAGLASSAEVDERHVQKQLGHASAEMT |
| | RKYQRRRDRFRVNLTKASGL |
| 1793 | WP_132665865.1 |
| | MMADNTNLNADMPRPAPSPSLPGHLQDLTDRARGYVEAASSANTRKAYASDWKHFAAWCRRSSLPLLPPH |
| | PQTIGLYITACSSGTAERGGKPNSVSTIGRRLSSLSWNYTQRGQQLDRKDRHIATVMAGIRNSHARPPVQ |
| | KEAVMAADIIAMIETLDRSTLRGMRDRAMLLVGYAGGLRRSEIVGLDVKADQTEDGRGWIEIFDKGMLVT |
| | LRGKTGWRQVEVGRGSSDATCPVVAVETWIRFAKLGHGPLFRRVTGQGKSIGAERLNDKEIARLVKRAVV |
| | AAGVRGDLSELERALKFSGHSLRAGLASSADVDERYVQKQLGHASAEMTRRYQRRRDRFRINLTKAAGL |
| 1794 | WP_069694293.1 |
| | MTSIALLSTESDTRRALQLDALAGILPLERRDQLAKILTDEDVATLRHLAREGMGENSLRALASDLAYLE |
| | AWSLASTGSALPWPAPEALALKFIAHHLWDPARRAEDFSHGMPADVEASLRAGDHLRSDGPHASSTVKRR |
| | LAHWATLHRWKGLESPLGTPAIRTGLRLAVRASAKPRRRKSKRAVTRDILDRLIATCQSDRLADSRDLAI |
| | LLVAFGSGGRRRSEVARLRLEQLTIEADVPLDPDDQDSARLPCMAIALGRTKNSQADDDARVLLIGPPVE |
| | ALREWLERAAISKGAVFRAIDRWEAIDDRALTPQAINLILKRRCAQAGLDPIAFSAHGLRSGYLTEAARN |
| | GVALPAAMRQSQHRSVQQAARYYNDADQALGKAARLAI |
| 1795 | RWE07715.1 |
| | MENPPSKPAKKDLPAADRPDGDLPDIVDLVMEMGRTAPRVPAHVEDLVETAKGYANAASSENTRDAYAKD |
| | WRHFTSSCRRTGFDPLPPDSKTIGLYISACARGEPKHGSPPLSVATIERRLSGLAWNFIQRGFVMDRADR |
| | HIATVLAGIRRKHAKPPRQKEAVLGDDLLAMIATLGHDLRSLRDRAILLLGFAGGLRRSEIVGLDVTREE |
| | TSDGAGWIEIFPDKGVLVTLRGKTGWREVEVGRGSSDLSCPVAALESWIRFGRIARGPLFRRIFKDNKTV |
| | DVGRLSDKHVARLVKKTALAAGVRSDLPEGERGLLFAGHSLRSGLASSAEIEERYVQKQLGHASAEMTRK |
| | YQRRRDRFRTNLTKASGL |
| 1796 | WP_011578806.1 |
| | MASHIDQNSENRTRHRSAQPQETPPGVDETAPAGSAEHDASIADDMPDIIDVVLEMGRAPEEPPDGAPSP |
| | ALPVVSTPRLPAHLDALADRARDYVEAASSSNTRRAYASDWKQFASWCRRQGVEMFPPDPQVVGLYIAAC |
| | ASGKATGDRKPNSVSTIERRLSALTWNYAQRGQPLDRKDRHIATVMAGIRNKHAAPPRQKEAVLPEDLIA |
| | MLETLDRGTLRGLRDRAMLLLGFAGGLRRSEVVGLDCGRDQTEDSSGWIEILDKGMLLRLRGKTGWREVE |
| | VGRGSSDTTCPVVALETWLKLARIAHGPLFRRVTGQGKKVGADRLNDQEVARLVKRTALAAGVRGDLPEG |
| | ERGMKFAGHSLRAGLASSAEVDERYVQKQLGHSSAEMTRKYQRRRDRFRVNLTKASGL |
| 1797 | RWD51833.1 |
| | MPSVDEARATASLVDQNPENRARRSSAQPQETATPVDQDAVDQTAAAPSAEPNGSSTDDLPDIIDVVMEM |
| | GRAPEQPSADAPSALPVVANARLPAHLDALADRARGYVEAASSANTRRAYASDWKQFASWCRRQGVEMFP |
| | PDPQVVGLYVTACASGKATGDKKPNSVSTIERRLSSLTWNYAQRGQPLDRKDRHIATVMAGIRNKHAAPP |
| | RQKEAVLPEDLIAMLETLDRGTLRGLRDRAMLLLGFGGGLRRSEVVGLDVGRDQTEDSSGWIEILDKGML |
| | LRLRGKTGWREVEVGRGSSDTTCPVVALETWLKLARIAHGPLFRRVTGQGKSVGADRLNDQEVARLVKRT |
| | ALAAGVRGDLPEGERGMMFAGHSLRAGLASSAEVDERYVQKQLGHTSAEMTRKYQRRRDRFRVNLTKASG |
| | L |
| 1798 | WP_096459680.1 |
| | MASLVDQNPEKHTQHGSAQPQETAPGVDQIAQAGSAGHDTPIADDLPDIIDVVLEMGRAPEEAPADTPSP |
| | LPVVANPRLPAHLDALAGRARDYVEAASSANTRRAYASDWKQFASWCRRQGVEMFPPDPQVVGLYITACA |
| | SGKAPGEKKPNSVSTIERRLSSLTWNYAQRGQPLDRKDRHIATVMAGIRNKHAAPPRQKEAVLPEDLIAM |
| | LETLDRGTLRGLRDRAMLLLGFAGGLRRSEVVGLDCGREQTEDSSGWIEILDKGMLLRLRGKTGWREVEV |
| | GRGSSDTTCPVVALETWLRLARIAHGPLFRRVTGQGKAVGADRLNDQEVARLVKRTALAAGVRGDLPEGE |
| | REKLFAGHSLRAGLASSAEVDERYVQKQLGHTSAEMTRRYQRRRDRFRVNLTKASGL |
| 1799 | RWD87033.1 |
| | MNQPTADDLPDIVDLVMEMGQPVRPPAHVEALVETAKGYAKAASSENTRNAYAKDWRHFTSWCRRQGFEP |
| | LPPDPKIIGLYISACAAGEPKHGAPALSVSTIERRLSGLAWNFTQRGFAIDRADRHISSVLAGIRRKHAK |
| | PPRQKEAVLSDDIKAMVNTLGHDLRSLRDRAILLLGFAGGLRRSEIVGLDVVRDDHSDGNGWIEFFPGQG |
| | VLVTLRGKTGWREVEVGRGASDQTCPVAALESWIRFGRIARGPLFRRIFKDNKTVDVERLSDKHVARLVK |
| | RTALAAGVRSDLPEGERAGLFSGHSLRAGLASSADIEERYVQKQLGHASAEMTRKYQRRRDRFRTNLTKA |
| | SGL |
| 1800 | WP_016210837.1 |
| | MSSKQIKKIMTAESRTEISTTLSSSSRQFLENTLAQATKRGYAADLKIFFAWAEAHQTAAIPATAETIAN |
| | FLADQASGVLSVWLRQESQLINGRPVSVATLRRRLAAIKYAHKLNKIEPSPTDTAEVRETLKGIRRTLGA |
| | KPNAKSALMSQDIQLLMRYIPETITGQRDRAILLLGFAGALRRSELTSLELSDIEVQENGMLVYIRSSKT |
| | DQEQQGQVIGIARSENKANCPVGAIEQWLQSSMILSGPIFRRIFANGKIAITTLSDRTIYNIVKNYCQLA |
| | GLDASRFGAHSLRRGFVTSAAKAKVDPFRIMAVTRHKRLETVKRYVDEANLINDYPGADLLK |
| 1801 | WP_073288106.1 |
| | MSEDLSLLPASDASHSLSHHLGRASAKVAGFLEAGLQGAANTERAYTSDLKSYVTFCEQHGFVAVPAEVE |
| | TITEYVAYLASEKVEPAPGGSRGKKKGQQPLTGPHALATIKRHLAAIRKAHQLAGHRLPATLDALNIVME |
| | GIARTLGKRQEQAQAFTVEELKQAIRRIDLETSAGLRDRALLLLGFAGAFRRSELVDLNIEQLEFTERAL |
| | LVHLAKSKTNQYRAVEDKAIFYAPNADYCPVRCLRAWLGLLGRTTGPLFVKIPRASPGQMAAPSDKRLSD |
| | ISINKLVQKRLGPDYSAHSLRVSFVTVAVLNGQSHKAIKNQTKQKTDAMIERYTQLNNVVSYNAAQALGL |
| 1802 | WP_092743158.1 |
| | MREDLTIVPASTVPPTVSTQLARASAKVASFLEVGLQGAANTERAYTSDLKSYVGFCERHGLRALPADVE |
| | TLTEYVAYLATEKPTPEPSDGGRGEKKKRKGQQPLTRPHSLATIKRHLAAIRKAHQLAGHRLPVTLDALN |
| | VVMEGIARTLGKRQDQAQAFTAEELKQAIRRIDLETSAGLRDRALLLLGFAGAFRRSELVELNIEQLEFT |
| | ERALLVHLAKSKTNQYGAVEDKAIFYAPTMDFCPVRCLRAWLNLLGRNTGPLFVKIPRATPGQMAAPSDK |
| | RLSDISINKLVQKRLGPAYSAHSLRVSFVTVAVLNGQSHKAIKNQTKQKTDAMIERYTQLNNVVSYNAAQ |
| | SLGL |
| 1803 | WP_026351576.1 |
| | MREDLSIVPASTVPPTVSTQLARASAKVAGFLEVGLQGAANTERAYTSDLKSYVGFCERHGLRALPADVE |
| | TLTEYVAYLATEKPIPEPGTGGRGEKKKRKGQQPLTRPHSLATIKRHLAAIRKAHQLAGHRLPATLDALN |
| | VVMEGIARTLGKRQDQAQAFTVEELKQAIRRIDLETSAGLRDRALLLLGFAGAFRRSELVELNIEQLEFT |
| | ERALLVHLTKSKTNQYGAVEDKAIFYAPTMDFCPVRCLRAWLNLLGRTTGPLFVKIPRAAAGQMAAASEK |
| | RLSDISINKLVQKRLGLGYSAHSLRVSFVTVAVLNGQSHKAIKNQTKQKTDAMIERYTQLNNVVSYNAAQ |
| | ALGL |
| 1804 | WP_089334212.1 |
| | MSEDLSLVASSPAGQSVGAQLARASAKVAGFLEVGLQGAANTERAYTSDLKSYVTFCEQHGFVAVPADVD |
| | TLTEYVAYLASEKPVSDTMGGGGKKKRKGQQPLTRPHSLATIKRHLAAIRKAHQLAGHRLPATLDALNIV |
| | MEGIARTLGKRQDQAPAFTVEELKQAIRRMDLETSAGLRDRALLLLGFAGAFRRSELVDLNIEQLDFTER |
| | ALLVHLAKSKTNQYGAVEDKAIFYAPNADYCPVRCLRAWLHLLGRTTGALFVKIPRAAPGQMAVPSDKRL |
| | SDISINKLVQKRLGPDYSAHSLRVSFVTVAVLNGQSHKAIKNQTKQKTDAMIERYTQLNNVVSYNAAQAL |
| | GL |
| 1805 | WP_086597010.1 |
| | MNEDLSLIPAANANQSISAQLARASAKVAGFLEAGLQGAANTERAYTADLKSYVAFCEQHGLQAVPADVD |
| | TLTEYVAYLASEKPEPAPGEGTRKKGQQPLTGPHALATIKRHLAAIRKAHQLAGYRLPATLDALNLVMEG |
| | ITRTLGKRQEQAQAFTVEELKQAIRRIDLDTSAGIRDRALLLLGFAGAFRRSELVELNIEQLEFTERALL |
| | VHLAKSKTNQYGAIEDKAIFYAPTMDYCPVRCLRAWLYLLGRTTGPLFVKIPRTIPGQLAVPSTKRLSDI |
| | SINKLVQKRLGPAYSAHSLRVSFVTTAVLNGQSHKAIKNQTKQKTDAMIEHYTQLHNVVSYNAAQALGL |
| 1806 | WP_092511277.1 |
| | MAGGLSLIDQEVVFIPDNPELNEDVLRNLHAFMKDKEAFADNTWQQLMKASRLWCQWCIGKGRPYLPVDA |
| | DYLRDYLWELHENGLAPATISNYAAMLNLLHRQAGLIPAGDSQKVKRILKKIHRVAIVHGEKAGQAIPFR |
| | IADLNQVDTAWQDSASLKERRNLAFLFVAYNTLLRISNLARLKVGDVTFNPDGSVMLHIGYTKTQVDGQG |
| | SIKALSPRASASLRHWLQVSGLIEHPDAYIFCRVHRSNQAIVATEKPMDEFNLSQVFSAAWSVVHGDKKA |
| | ARNKGRYATWTGHSARVGAAQDMTESGYSLAQIMHEGAWKKPETVLGYIRNIEAKKSVMIELVEGKS |
| 1807 | WP_055739375.1 |
| | MEDRLQDFIHFMVVEKGLSKNTILSYERDLKNYLLYIQKVEQITSLNDITRVHIVHFLHHLKKQGKSAKT |
| | LARHVASVRSFHQFLLREKATEHDPSVHIESPQIEKTLPKVLNLSEVEALLESPDEDSPLGIRDRAMLEL |
| | LYATGIRVSELIQLNLDDLHLTMGFIRCIGKGNKERIIPIGKTASNVIEKYISIGRPKLKSKQNSTEALF |
| | LNHHGNRLTRQGFWKILKGLAKKANIEKDLTPHTLRHSFATHLLENGADLRAVQEMLGHADISTTQIYTH |
| | VTKIRLKDVYSKHHPRA |
| 1808 | WP_058066517.1 |
| | MSSTTQAKASLEAELAKHPGIEIHGNSIRVVFMWRRRRYRETLGLPLTKANIKHAALLRAAVLHDIKIGT |
| | FDYGRHFPNSRNATNFSNTKDERLHALLERYKPLKAVDITTETQSRYFAALDICVDLLGGNRLGSILLPE |
| | DIQKLRVELIAGRATSTTNHYLATLAGFLNWCESNGYCRKGLAEACTRFTMTDRDPDPLTKSEFEALLDK |
| | GCLHPMDHAAITLAVYTGLRPGELCALAREDVDMANGLIHVNRSITSSGTFKLPKTGKKRTVMLFPPALE |
| | ACRVLLGIKHGIAPQKLAIELNRHESVQETVTPLLTPLVQARRKQINTWFVPTAWNTKWHNIQRRAKIRP |
| | RRPYQTRHTYACWCLVARGNLAFIAKQMGHKDFTMLVQVYAKWMDDESPNELSSIWAGMSR |
| 1809 | WP_002187515.1 |
| | MSISSNVILISDHRKKKYKKSLNNDSGSMFGKGLTDEMMSYLTKEFANPVSERAYRNRAIFLILSQTALR |
| | AKETVNLRFSDLLKAPTNETLARYVKKGGRIAYSVISESCLKSVQEYHSKFNLKSDYFFLSLPRRNQNWR |
| | SNLSTRGLQLIVNSWNVRTCSGRISRPHCFRHTAGTKLLETSGSIAAQLTLGHSSPIITSKYYTKRYYNA |
| | SMFLTWE |
| 1810 | WP_127622166.1 |
| | MIDNQRAARSDSQAVHRRAEELDALDAILPFDRRDQLSALLTDDDVATLKHLAQEGMGENTLRALSSDLG |
| | YLEAWCRLATGDPLPWPAPEALLLKFVAHHLWDPVKRAEDPAHGMPADVEAGLRAERLLRSPGPHAPGTV |
| | RRRLTSWSILTRWRGLAGAFGAPSLKSALRLAVRASARPRQRKSKKAVTVDILVQLLQACAGDRLVDVRD |
| | RALLLAAFASGGRRRSEVAALRVEDLADEELVRADPSDKTSPPLPCLSIRLGRTKTTTADENEHVLLIGR |
| | PVAALKTWLAEAQIKDGPVFRCIDRWGNIDRRALTPQSVNLILKARCEQAGLDPALFSAHGLRSGYLTEA |
| | ANRGIPLPEAMQQSLHKSVTQAASYYNNAERKNGRAARLIV |
| 1811 | WP_101200924.1 |
| | MTDLMSVSDISDETVRSQVLANLEEFKHDLLDDMASNTKRAYLSDFEHYLSFCLKHGLVSMSDDWRVTKD |
| | TIKTYFVSLMASDLKNASIKRKLSSIKFFIGIAELPDPFKHSKLLRDFITNKLKKKPAAQTQANPVTAEV |
| | LVALNETFNPLSLLDIRNKLLVNLAFDSLFRASNLAEIEVAHIDREHGSVFASYSKTDQEGQGSYGYISP |
| | KTIILLDEWLNASGITERFIFRTLSPKQTVQQKTMGYQAIYKTFKNFGGSRYLDNKISYSCHSTRVGATV |
| | SMTEQNRPLIKIIQAGNWKSERMAIRYGQRTNVAKGGMVDI1 |
| 1812 | WP_068331637.1 |
| | METDTALLANPVGHGLAHHTGAAARYVEAGLNGAPNTTRAYTAHLKRFGGWCAAHGHQPLPASVDALVGF |
| | CTHLAEAGKKVGTLQQHCAAISKAHAVRGVDSPTDDKQFKIFMDGVRRVHGVRQKQAPAFSLAQFKQLVR |
| | GLDTTTVAGLRDRAILLLGFTGAFRRSELTALNVQDLRFTEDCLVVSLGRSKTNQLGDYEEKAIFYSPES |
| | AVCPIRSLKAWLAQLERSEGPVFVMLRKGNRLTTNRLSDQTINTLVQRYLGAGYTAHSLRASFVTVAKLN |
| | GADDSKIMNQTKHKTSAMIRRYTRLDNVQQHNAAKELGL |
| 1813 | WP_023274785.1 |
| | MKIPKPRKRGDSFRIELMYEGRRISATRDTEKECEQWAALKLLEFKTGKAQEEKGIKPSFPFKKLCEKYF |
| | LEKGSKLKSSHVIRNKLDNLERITGELANKSIYDFKPNDIVRWRNRRVLEVKSSTALREFAMFSAIFTYA |
| | QKELFLIENNVWNTVVKPDKGKGRSQRISPEDQEKIFKRSKWDNETAPFYSQHYVGWSLLFALETAMRQG |
| | EILAMKRKDVRDGFIHLPITKNGESRDVPLSKEAKRLLSLLPVENDILVPVKVKTFKRTWIRMRDEAGLS |
| | HINFHDTRHEAITRMVRNRKLPVEVLAKITGHKTINILINTYYNPNYECKFFPRVSHDIHQ |
| 1814 | WP_018409463.1 |
| | MTKIDDDLESGGAPLAERPSAPHLAALSEKARDYARNARSDNTRRAYDADWRQFAAWLRRQGLDPLPPEP |
| | QTVGLYLTACMEGVLGREPVSVATLERRLAGICWHYRQCGAPLDTSDRHIATVLAGIRRAHSRPPLQKEA |
| | IFADELLAMLSVLEMDLRGIRDRAILAIGFSGGLRRSEIVGLDCGPDQTEDGAGWVEIFPPAGPGNEGGA |
| | VLQISGKTGWREVEIGRGSRPETCPVALLETWMRLGRISHGPLFRPIARKNGGVSSERLTDKHVARLVQK |
| | TALAAGIRGDLTEGERRLAFGGHSLRAGLASSAQIEEAHVQKHLGHASAEMTRRYQRKRDRFRVNLTKAA |
| | GL |
| 1815 | WP_010305236.1 |
| | MGYKIKKFIMSSGERGHLILDKETELPVYYQNLFLTENVRNRNATASTVEVVATNLLIFSNFLDSRKINI |
| | VERIEAKKYLSIAEINDLIRYAKQRFDKQKITNIRQMNKMFIAKRTFSYRIHVFSSYLKWLCILVHSTKG |
| | IHDRYEVDNFIESIKAYIPRKSSLNMNDRSDKSLDEGEIRILFNLLKVDGNNNPFQKDVQIRNRLIFSLL |
| | FNLGLRAGELLNLKIDDFDLRDNTLSIIRRHDSKEDRRSYQPLVKTGERVIPLSDELASDIFRYISDSRE |
| | KMTKRKKHNFLLVAHYTGKTAGEPLSISAYEKIISTLKRASPELSKLSGHRLRHSWNYIYSKEMDVSNLE |
| | FGRKKELRNYCQGWSKGSKMSENYNFKYISQQEKEVILRIYGSINKIISGA |
| 1816 | WP_008737017.1 |
| | MTTPLSVRAIESMRPGDSPRTDVGETQGLRVTCAKSGVRTFIYRYRSPETGKLVQLKLGHYPGLKLAEAR |
| | MQVVRMKELQRAGVCPKAQQERELAAQREEEEKARREQEAAAFLVADLIELYLTEVIEDRMIKDARTGKP |
| | KRVAGSRKPKGQAEVRRTLYNDPVRVLGDMPAGEVTRKHVVDLVRKILARGANVQAGNVLRELTAAYEYA |
| | IAMEKLPEDFANPAMLAKGSLRTARVKLSSEKGRRALSDEDLRALLAWLPGSGFSVTQKNVIRLTLWTGC |
| | RTGEVCEAEWRDVDLEKGVWHMRDSKNGAERYVQLSRQAVEFLRQLKLNGTTYVFPSSRSGRPIQQKSLS |
| | EAKWQLKHPEQVQNRRVYRPEQRWLTTIEDWSPHDLRRTVRTGLSRLGCPSEVAEAVLGHSRKGIEGTYD |
| | LHRYEDQCKVWLQKWADHLDTLLRQKG |
| 1817 | WP_006526094.1 |
| | MRQLRRLQRTKGYLRKTDDKHGREIVKPFIKSDFDEMVRCCLNHRDKHNPSSWKYRVWYRNYILLILGVN |
| | TGNRIETLIELTPRDIAGGQYTCKEMKTGKVQQFNMNADVYATVREYIERYNIQMNEYIFESRQGFKGYP |
| | ITRQQAWRIIKQLADEAGIKYPVACHSLRKSYGRWYWDSTHDLLTTQKLLMHESAAETMLYIMLEPSDIQ |
| | EVRESINHTEKWG |
| 1818 | WP_127657123.1 |
| | MPNLVTPRETNLDDEALEALSDLFVRGTPANTIRAYERDLAYITAWKMAAFGTDLAWPEEEAVAMRFVLD |
| | HSRDLQDISGDAARVAQSLISQGLRRSLECPAPSTLDRRIASWRSFHRMRNLPSPFDAPLIRQARSKARR |
| | AAGRPAAPKSANPITREIVDEMCAAAGPGLRGIRDRAILLLGWASGGRRRSEIATLRREDVDLGDFDSDG |
| | IVWLRLRDTKTTRQEQTPKLVLKGRAARAVTAWIDAAEIRDGALFRKIGTTGRPGTRALSPAGIGQIVKR |
| | CLEQSGRGADFASAHGLRSGFLTQAALDGAPLQAAMRLSLHKSAAQAQAYYGDVEITDNPATDLLDKS |
| 1819 | WP_071857225.1 |
| | MNQQEANRRMEEEIQFFPWFIQNYFRKKSGDQYSSITLYEYAKEYRRFLNWLIQESFSSADKISEVTLTE |
| | FANLWPEDLEFYKAHLVKAPKILKETTQKRLEENDQSLPLRQNATVQRGITALRSLFNYLNDAVDRNSGK |
| | PYLEHNMMAKVANVKDNKTMAERGAAIEKKLFLDEEAIDFLDYIEHRYIDTLETRQAITAFKKNQVRDLA |
| | IIALFLGTGMRLSELVNMNVQDLDLTSGEARVYRKGGKWDMVVISSIAMEFLTNYLAQRENLYQPAETET |
| | ALFLTRYRGKAKRIASGAVEAMVGKYSESFKIKISPHKLRHSVATQLYSKTNSLIQVAEQLGQSGTSATT |
| | VYTHIAGKQKRDAMNDLWT |
| 1820 | WP_107676128.1 |
| | MQNPPANTPKIDDSADSALPAGVELVVEMDAASRPARLEALVETATAYANAASSENTRDAYAKDWRHFTT |
| | WCRREGFEPMPPSSQVIGLYIGACASGDPKRNTPALSVATIERRLSGLAWNFTQRGIPMDRSNRHIATVL |
| | AGIRRKHAKPPRQKEAVLGEDIKAMVDTLGHHLRGLRDRAILLLGFAGGLRRSEIVGLDIVRDDHSDGHG |
| | WIEIFPSQGVLVTLRGKTGWRQVEVGRGASEQTCPVVALESWIRFGRIVRGPLFRRIFKDNKTVDVERLS |
| | DKHVARLVKQTALAAGVRSDLPEGERALLFAGHSLRAGLASSAEIEERYVQKQLGHASAEMTRKYQRRRD |
| | RFRTNLTKASGL |
| 1821 | WP_003132298.1 |
| | MTITKNKNGTWRVDISDGINPLTGIQGRHRKYDCKTKKEAIEYEAKYRLEELGEFKRKDKLSIDSLYALL |
| | KKEDVLRGNRQSTKDTQDSYYRIYVSKFFQNADMRLVKTSDIKAFRDWLIKTPSVKGGNLSASNINTIMI |
| | FVGKLFDISMMNDLRKDNPCKALKRLPQQHKEMFYYTPEQFKQFISLFDESEYHFQLLYKILMFTGARIG |
| | EALALTWEQINLEIGYIDIKSSAHYRKSKVTIAETKTTQSIRRIYIHKALIDELSKWKQRQFQLLIKYIS |
| | TPEQLQIYQNTPKVLTAPDVSNFKKEKLKKRAELINLKLIRNHDFRHSHAAFLISQGLRKGEGKDYLFFT |
| | LMKRLGHSSITTTINTYSHLFPTQQKEIANAFDDF |

In some embodiments, a recombinase polypeptide (e.g., comprised in a system or cell as described herein) comprises an amino acid sequence as listed in Table 2, or an amino acid sequence having at least 70%, 75%, 80%, 85%, 90%, 95%, 96%, 97%, 98%, 99%, or 100% sequence identity thereto, or having no more than 1, 2, 3, 4, 5, 6, 7, 8, 9, 10, 15, 20, 25, 30, 35, 40, 45, or 50 sequence alterations (e.g., substitutions, insertions, or deletions) relative thereto. In some embodiments, a recombinase polypeptide (e.g., comprised in a system or cell as described herein), or a portion thereof, has at least 70%, 75%, 80%, 85%, 90%, 95%, 96%, 97%, 98%, 99%, or 100% sequence identity to the amino acid sequence of a DNA binding domain, recombinase normal, N-terminal domain, and/or C-terminal domain of a recombinase polypeptide as listed in Table 2, or having no more than 1, 2, 3, 4, 5, 6, 7, 8, 9, or 10 sequence alterations (e.g., substitutions, insertions, or deletions) relative thereto. In some embodiments, a recombinase polypeptide (e.g., comprised in a system or cell as described herein) has one or more of the DNA binding activity and/or the recombinase activity of a recombinase polypeptide comprising an amino acid sequence as listed in Table 2, or an amino acid sequence having at least 70%, 75%, 80%, 85%, 90%, 95%, 96%, 97%, 98%, 99%, or 100% sequence identity thereto, or an amino acid sequence having no more than 1, 2, 3, 4, 5, 6, 7, 8, 9, 10, 15, 20, 25, 30, 35, 40, 45, or 50 sequence alterations (e.g., substitutions, insertions, or deletions) relative thereto.

In some embodiments, an insert DNA (e.g., comprised in a system or cell as described herein) comprises a nucleic acid recognition sequence as listed in column 2 or 3 of Table 1, or a nucleic acid sequence having at least 70%, 75%, 80%, 85%, 90%, 95%, 96%, 97%, 98%, or 99% identity thereto, or having no more than 1, 2, 3, 4, 5, 6, 7, or 8 sequence alterations (e.g., substitutions, insertions, or deletions) relative thereto. In some embodiments, an insert DNA (e.g., comprised in a system or cell as described herein) comprises one or more (e.g., both) parapalindromic sequences of a nucleic acid recognition sequence as listed in column 2 or 3 of Table 1, or a nucleic acid sequence having at least 70%, 75%, 80%, 85%, 90%, 95%, 96%, 97%, 98%, or 99% identity thereto, or having no more than 1, 2, 3, 4, 5, 6, 7, or 8 sequence alterations (e.g., substitutions, insertions, or deletions) relative thereto. In some embodiments, an insert DNA (e.g., comprised in a system or cell as described herein) comprises a spacer (e.g., a core sequence) of a nucleic acid recognition sequence as listed in column 3 of Table 1, or a nucleic acid sequence having at least 70%, 75%, 80%, 85%, 90%, 95%, 96%, 97%, 98%, or 99% identity thereto, or having no more than 1, 2, 3, 4, 5, 6, 7, or 8 sequence alterations (e.g., substitutions, insertions, or deletions) relative thereto. In certain embodiments, the insert DNA further comprises a heterologous object sequence.

In some embodiments, an insert DNA (e.g., comprised in a system or cell as described herein) comprises a nucleic acid recognition sequence as listed in column 2 or 3 of Table 1, or a nucleic acid sequence having at least 70%, 75%, 80%, 85%, 90%, 95%, 96%, 97%, 98%, or 99% identity thereto, or having no more than 1, 2, 3, 4, 5, 6, 7, or 8 sequence alterations (e.g., substitutions, insertions, or deletions) relative thereto, that is the cognate to a human recognition sequence (e.g., as listed in column 3 of Table 1, e.g., in the same row as that listing the nucleic acid recognition sequence in column 2), or a nucleic acid sequence having at least 70%, 75%, 80%, 85%, 90%, 95%, 96%, 97%, 98%, or 99% identity thereto, or having no more than 1, 2, 3, 4, 5, 6, 7, or 8 sequence alterations (e.g., substitutions, insertions, or deletions) relative thereto. In certain embodiments, the cognate human recognition sequence is located in the human genome at a position listed in column 4 of Table 1 (e.g., corresponding to the cognate human recognition sequence listed in the same row in column 3).

In some embodiments, an insert DNA or recombinase polypeptide used in a composition or method described herein directs insertion of a heterologous object sequence into a position having a safe harbor score of at least 3, 4, 5, 6, 7, or 8. In some embodiments, an insert DNA or recombinase polypeptide used in a composition or method described herein directs insertion of a heterologous object sequence into a genomic safe harbor site that is unique, with 1 copy in the human genome. By way of example, a unique site may be present at 1 copy in the haploid human genome, such that a diploid cell may comprise 2 copies of the site, situated on a homologous chromosome pair. As a further example, a unique site may be present at 1 copy in the diploid human genome, such that a diploid cell comprises 1 copy of the site, situated on only one chromosome of a homologous chromosome pair.

In some embodiments the three base pairs in the parapalindromic sequence directly adjacent to the core sequence (a “core adjacent motif”) comprise AAA, AGA, ATA, or TAA. In some embodiments, the core adjacent motif comprises at least one A (e.g., comprises 2 or 3 As). In some embodiments, the core adjacent motif is ANA or NAA (where N is any nucleotide). In some embodiments, a DNA recognition site described herein comprises a first core adjacent motif in the first parapalindromic sequence and a second core adjacent motif in the second parapalindromic sequence. In some embodiments, the first core adjacent motif and the second core adjacent motif have the same nucleotide sequence, and in other embodiments, the first core adjacent motif and the second core adjacent motif have different sequences.

In some embodiments, the DNA recognition sequence on the insert DNA has 0, 1, 2, 3, 4, 5, 6, 7, 8, 9, 10, 11, 12, 13, 14, 15 or more mismatches as compared to the human DNA recognition sequence. Without wishing to be bound by theory, it is contemplated that the mismatches between the DNA recognition sequences may, in some embodiments, bias recombinase activity towards integration over excision, for example, as described in Araki et al., Nucleic Acids Research, 1997, Vol. 25, No. 4, 868-872, incorporated herein by reference in its entirety. In some embodiments, the DNA recognition sequences on the insert DNA and/or the human DNA recognition sequences each comprise 1, 2, 3, 4, 5, 6, 7, 8, 9, 10, 11, 12, 13, 14, 15 or more mismatches as compared to the native recognition sequence recognized by the recombinase polypeptide. In certain embodiments, recombination between the insert DNA and the human DNA recognition sequence results in the formation of an integrated nucleic acid molecule comprising two recognition sequences flanking the integrated sequence (e.g., the heterologous object sequence). In certain embodiments, one or both of the two recognition sequences of the integrated nucleic acid molecule comprises 1, 2, 3, 4, 5, 6, 7, 8, 9, 10, 11, 12, 13, 14, 15 or more mismatches as compared to one or more of (e.g., one, two, or all three of): (i) the native recognition sequence, (ii) the recognition sequence on the insert DNA, and/or (iii) the human DNA recognition sequence. In some embodiments the mismatches are all present on the same parapalindromic sequence. In some embodiments the mismatches are present on different parapalindromic sequences. In embodiments, one or both of the two recognition sequences of the integrated nucleic acid molecule comprises 1, 2, 3, 4, 5, 6, 7, 8, 9, 10, 11, 12, 13, 14, 15 or more mismatches as compared to the native recognition sequence. In some embodiments the mismatches are present in the core sequence. It is contemplated that, in some embodiments, these differences between the recognition sequence(s) of the integrated nucleic acid molecule and the native recognition sequence, the insert DNA recognition sequence, and/or the human DNA recognition sequence result in reduced binding affinity between the recombinase polypeptide and the recognition sequences of the integrated nucleic acid molecule, compared to the recognition sequence(s) of the integrated nucleic acid molecule and the native recognition sequence.

In some embodiments, a human recognition sequence (e.g., a human DNA recognition sequence, e.g., as listed in column 3 of Table 1) is located in or near (e.g., within 1, 2, 3, 4, 5, 10, 15, 20, 30, 40, 50, 75, 100, 150, 200, 300, 400, 500, 600, 700, 800, 900, 1000, 2000, 3000, 4000, 5000, or 10,000 nucleotides of) a genomic safe harbor site. In some embodiments, the human recognition sequence is located at a position in the genome that meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA+/−25 kb); (vii) is not in a copy number variable region; (viii) is in open chromatin; and/or (ix) is unique, with 1 copy in the human genome. In some embodiments, a genomic location listed in column 4 of Table 1 is located in or near (e.g., within 1, 2, 3, 4, 5, 10, 15, 20, 30, 40, 50, 75, 100, 150, 200, 300, 400, 500, 600, 700, 800, 900, 1000, 2000, 3000, 4000, 5000, or 10,000 nucleotides of) a genomic safe harbor site. In some embodiments, a genomic location listed in column 4 of Table 1 is at a position in the genome that meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA+/−25 kb); (vii) is not in a copy number variable region; (viii) is in open chromatin; and/or (ix) is unique, with 1 copy in the human genome.

In embodiments, a cell or system as described herein comprises one or more of (e.g., 1, 2, or 3 of): (i) a recombinase polypeptide as listed in a single row of column 1 of Table 1 or 2, or an amino acid sequence having at least 70%, 75%, 80%, 85%, 90%, 95%, 96%, 97%, 98%, 99%, or 100% sequence identity thereto; (ii) an insert DNA comprising a DNA recognition sequence as listed in column 2 and the same row of Table 1, or a nucleic acid sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto, optionally wherein the insert DNA further comprises an object sequence (e.g., a heterologous object sequence); and/or (iii) a genome comprising a human DNA recognition sequence sequence as listed in column 3 and the same row of Table 1, or a nucleic acid sequence having at least 70%, 75%, 80%, 85%, 90%, 95%, 96%, 97%, 98%, or 99% identity thereto, or having no more than 1, 2, 3, or 4 sequence alterations (e.g., substitutions, insertions, or deletions) relative thereto; preferably wherein the human DNA recognition sequence is located in the genome at the location listed in column 4 and the same row of Table 1 corresponding to the listing of the human DNA recognition sequence.

In some embodiments, the protein component(s) of a Gene Writing™ system as described herein may be pre-associated with a template (e.g., a DNA template). For example, in some embodiments, the Gene Writer™ polypeptide may be first combined with the DNA template to form a deoxyribonucleoprotein (DNP) complex. In some embodiments, the DNP may be delivered to cells via, e.g., transfection, nucleofection, virus, vesicle, LNP, exosome, fusosome. Additional description of DNP delivery is found, for example, in Guha and Calos J Mol Biol (2020), which is herein incorporated by reference in its entirety.

In some embodiments, a polypeptide described herein comprises one or more (e.g., 2, 3, 4, 5) nuclear targeting sequences, for example a nuclear localization sequence (NLS). In some embodiments, the NLS is a bipartite NLS. In some embodiments, an NLS facilitates the import of a protein comprising an NLS into the cell nucleus. In some embodiments, the NLS is fused to the N-terminus of a Gene Writer described herein. In some embodiments, the NLS is fused to the C-terminus of the Gene Writer. In some embodiments, the NLS is fused to the N-terminus or the C-terminus of a Cas domain. In some embodiments, a linker sequence is disposed between the NLS and the neighboring domain of the Gene Writer.

In some embodiments, an NLS comprises the amino acid sequence MDSLLMNRRKFLYQFKNVRWAKGRRETYLC (SEQ ID NO: 1822), PKKRKVEGADKRTADGSEFESPKKKRKV (SEQ ID NO: 1823), RKSGKIAAIWKRPRKPKKKRKV KRTADGSEFESPKKKRKV (SEQ ID NO: 1824), KKTELQTTNAENKTKKL (SEQ ID NO: 1825), or KRGINDRNFWRGENGRKTR (SEQ ID NO: 1826), KRPAATKKAGQAKKKK (SEQ ID NO: 1827), or a functional fragment or variant thereof. Exemplary NLS sequences are also described in PCT/EP2000/011690, the contents of which are incorporated herein by reference for their disclosure of exemplary nuclear localization sequences.

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK (SEQ ID NO: 1828), wherein the spacer is bracketed. Another exemplary bipartite NLS has the sequence PKKKRKVEGADKRTADGSEFESPKKKRKV (SEQ ID NO: 1829). Exemplary NLSs are described in International Application WO2020051561, which is herein incorporated by reference in its entirety, including for its disclosures regarding nuclear localization sequences.

DNA Binding Domains

In some embodiments, a recombinase polypeptide (e.g., comprised in a system or cell as described herein), e.g., a tyrosine recombinase, comprises a DNA binding domain (e.g., a target binding domain or a template binding domain).

In some embodiments, a recombinase polypeptide described herein may be redirected to a defined target site in the human genome. In some embodiments, a recombinase described herein may be fused to a heterologous domain, e.g., a heterologous DNA binding domain. In some embodiments, a recombinase may be fused to a heterologous DNA binding domain, e.g., a DNA binding domain from a zinc finger, TAL, meganuclease, transcription factor, or sequence-guided DNA binding element. In some embodiments, a recombinase may be fused to a DNA binding domain from a sequence-guided DNA binding element, e.g., a CRISPR-associated (Cas) DNA binding element, e.g., a Cas9. In some embodiments, a DNA binding element fused to a recombinase domain may contain mutations inactivating other catalytic functions, e.g., mutations inactivating endonuclease activity, e.g., mutations creating an inactivated meganuclease or partially or completely inactivate Cas protein, e.g., mutations creating a nickase Cas9 or dead Cas9 (dCas9).

In some embodiments, a DNA binding domain comprises a Streptococcus pyogenes Cas9 (SpCas9) or a functional fragment or variant thereof. In some embodiments, the DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T1337I, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T1337I, T1337V, T1337F, T1337S, T1337N, T1337K, T1337R, T1337H, T1337Q, and T1337M, or corresponding amino acid substitutions thereto.

In some embodiments, the DNA binding domain comprises a Cas domain, e.g., a Cas9 domain. In embodiments, the DNA binding domain comprises a nuclease-active Cas domain, a Cas nickase (nCas) domain, or a nuclease-inactive Cas (dCas) domain. In embodiments, the DNA binding domain comprises a nuclease-active Cas9 domain, a Cas9 nickase (nCas9) domain, or a nuclease-inactive Cas9 (dCas9) domain. In some embodiments, the DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the DNA binding domain comprises a Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the DNA binding domain comprises an S. pyogenes or an S. thermophilus Cas9, or a functional fragment thereof. In some embodiments, the DNA binding domain comprises a Cas9 sequence, e.g., as described in Chylinski, Rhun, and Charpentier (2013) RNA Biology 10:5, 726-737; incorporated herein by reference. In some embodiments, the DNA binding domain comprises the HNH nuclease subdomain and/or the RuvC1 subdomain of a Cas, e.g., Cas9, e.g., as described herein, or a variant thereof. In some embodiments, the DNA binding domain comprises Cas12a/Cpf1, Cas12b/C2c1, Cas12c/C2c3, Cas12d/CasY, Cas12e/CasX, Cas12g, Cas12h, or Cas12i. In some embodiments, the DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the DNA binding domain comprises spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the DNA-binding domain comprises an amino acid sequence as listed in Table 3 below, or an amino acid sequence having at least 80%, 85%, 90%, 95%, 96%, 97%, 98%, or 99% sequence identity thereto. In some embodiments, the DNA-binding domain comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 differences (e.g., mutations) relative to any of the amino acid sequences described herein.

TABLE 3
Each of the Reference Sequences are incorporated by reference in their entirety

| Name | Amino Acid Sequence or Reference Sequence |
|---|---|
| Streptococcus pyogenes | |
| Cas9 | |
| Exemplary Linker | SGSETPGTSESATPES (SEQ ID NO: 1830) |
| Exemplary Linker Motif | (SGGS)ₙ (SEQ ID NO: 1831) |
| Exemplary Linker Motif | (GGGS)ₙ (SEQ ID NO: 1832) |
| Exemplary Linker Motif | (GGGGS)ₙ (SEQ ID NO: 1833) |
| Exemplary Linker Motif | (G)ₙ |
| Exemplary Linker Motif | (EAAAK)ₙ (SEQ ID NO: 1834) |
| Exemplary Linker Motif | (GGS)ₙ |
| Exemplary Linker Motif | (XP)ₙ |
| Cas9 from Streptococcus | NCBI Reference Sequence: NC_002737.2 and Uniprot |
| pyogenes | Reference Sequence: Q99ZW2 |
| Cas9 from Corynebacterium | NCBI Refs: NC_015683.1, NC_017317.1 |
| ulcerans | |
| Cas9 from Corynebacterium | NCBI Refs: NC_016782.1, NC_016786.1 |
| diphtheria | |
| Cas9 from Spiroplasma | NCBI Ref: NC_021284.1 |
| syrphidicola | |
| Cas9 from Prevotella | NCBI Ref: NC_017861.1 |
| intermedia | |
| Cas9 from Spiroplasma | NCBI Ref: NC_021846.1 |
| taiwanense | |
| Cas9 from Streptococcus | NCBI Ref: NC_021314.1 |
| iniae | |
| Cas9 from Belliella baltica | NCBI Ref: NC_018010.1 |
| Cas9 from Psychroflexus | NCBI Ref: NC_018721.1 |
| torquisI | |
| Cas9 from Streptococcus | NCBI Ref: YP_820832.1 |
| thermophilus | |
| Cas9 from Listeria innocua | NCBI Ref: NP_472073.1 |
| Cas9 from Campylobacter | NCBI Ref: YP_002344900.1 |
| jejuni | |
| Cas9 from Neisseria | NCBI Ref: YP_002342100.1 |
| meningitidis | |
| dCas9 (D10A and H840A) | |
| Catalytically inactive Cas9 | |
| (dCas9) | |
| Cas9 nickase (nCas9) | |
| Catalytically active Cas9 | |
| CasY | ((ncbi.nlm.nih.gov/protein/APG80656.1) |
| | >APG80656.1 CRISPR-associated protein CasY [uncultured |
| | Parcubacteria group bacterium]) |
| CasX | uniprot.org/uniprot/F0NN87; uniprot.org/uniprot/F0NH53 |
| CasX | >tr\|F0NH53\|F0NH53_SULIR CRISPR associated protein, Casx |
| | OS = Sulfolobus islandicus (strain REY15A) GN = SiRe_0771 |
| | PE = 4 SV = 1 |
| Deltaproteobacteria CasX | |
| Cas12b/C2c1 | ((uniprot.org/uniprot/T0D7A2#2) sp\|T0D7A2\|C2C1_ALIAG |
| | CRISPR- associated endonuclease C2c1 OS = Alicyclobacillus |
| | acido-terrestris (strain ATCC 49025/DSM 3922/CIP 106132/ |
| | NCIMB 13137/GD3B) GN = c2c1 PE = 1 SV = 1) |
| BhCas12b (Bacillus | NCBI Reference Sequence: WP_095142515 |
| hisashii) | |
| BvCas12b (Bacillus sp. V3- | NCBI Reference Sequence: WP_101661451.1 |
| 13) | |
| Wild-type Francisella | |
| novicida Cpf1 | |
| Francisella novicida Cpf1 | |
| D917A | |
| Francisella novicida Cpf1 | |
| E1006A | |
| Francisella novicida Cpf1 | |
| D1255A | |
| Francisella novicida Cpf1 | |
| D917A/E1006A | |
| Francisella novicida Cpf1 | |
| D917A/D1255A | |
| Francisella novicida Cpf1 | |
| E1006A/D1255A | |
| Francisella novicida Cpf1 | |
| D917A/E1006A | |
| SaCas9 | |
| SaCas9n | |
| PAM-binding SpCas9 | |
| PAM-binding SpCas9n | |
| PAM-binding SpEQR Cas9 | |
| PAM-binding SpVQR Cas9 | |
| PAM-binding SpVRER | |
| Cas9 | |
| PAM-binding SpVRQR | |
| Cas9 | |
| SpyMacCas9 | |

In some embodiments, the Cas polypeptide binds a gRNA that directs DNA binding. In some embodiments, the gRNA comprises, e.g., from 5′ to 3′ (1) a gRNA spacer; (2) a gRNA scaffold. In some embodiments:
(1) Is a Cas9 spacer of ˜18-22 nt, e.g., is 20 nt
(2) Is a gRNA scaffold comprising one or more hairpin loops, e.g., 1, 2, of 3 loops for associating the template with a nickase Cas9 domain. In some embodiments, the gRNA scaffold carries the sequence, from 5′ to 3′,

(SEQ ID NO: 1835)
GTTTTAGAGCTAGAAATAGCAAGTTAAAATAAGGCTAGTCCGTTATCAA
CTTGAAAAAGTGGGACCGAGTCGGTCC.

In some embodiments, a Gene Writing system described herein is used to make an edit in HEK293, K562, U20S, or HeLa cells. In some embodiment, a Gene Writing system is used to make an edit in primary cells, e.g., primary cortical neurons from E18.5 mice.

In some embodiments, a system or method described herein involves a CRISPR DNA targeting enzyme or system described in US Pat. App. Pub. No. 20200063126, 20190002889, or 20190002875 (each of which is incorporated by reference herein in its entirety) or a functional fragment or variant thereof. For instance, in some embodiments, a GeneWriter polypeptide or Cas endonuclease described herein comprises a polypeptide sequence of any of the applications mentioned in this paragraph, and in some embodiments a guide RNA comprises a nucleic acid sequence of any of the applications mentioned in this paragraph.

In some embodiments, the DNA binding domain (e.g., a target binding domain or a template binding domain) comprises a meganuclease domain, or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40(2):847-860 (2012), incorporated herein by reference in its entirety. In embodiments, the DNA binding domain comprises one or more modifications relative to a wild-type DNA binding domain, e.g., a modification via directed evolution, e.g., phage-assisted continuous evolution (PACE).

Inteins

In some embodiments, as described in more detail below, Intein-N may be fused to the N-terminal portion of a polypeptide (e.g., a Gene Writer polypeptide) described herein, e.g., at a first domain. In embodiments, intein-C may be fused to the C-terminal portion of the polypeptide described herein (e.g., at a second domain), e.g., for the joining of the N-terminal portion to the C-terminal portion, thereby joining the first and second domains. In some embodiments, the first and second domains are each independently chosen from a DNA binding domain and a catalytic domain, e.g., a recombinase domain. In some embodiments, a single domain is split using the intein strategy described herein, e.g., a DNA binding domain, e.g., a dCas9 domain.

In some embodiments, a system or method described herein involves an intein that is a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein inons.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem. 289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C (e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, Intein-N and intein-C may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]˜C. In some embodiments, an intein-C is fused to the N-terminus of the C-terminal portion of the split Cas9, i.e., to form a structure of N-[intein-C]˜[C-terminal portion of the split Cas9]-C. The mechanism of intein-mediated protein splicing for joining the proteins the inteins are fused to (e.g., split Cas9) is described in Shah et al., Chem Sci. 2014; 5(1):446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a Gene Writer polypeptide, e.g., as described herein, is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, a Gene Writer polypeptide (e.g., comprising a nickase Cas9 domain) is fused to intein-N and a polypeptide comprising a polymerase domainis fused to an intein-C.

Exemplary nucleotide and amino acid sequences of interns are provided below:

DnaE Intein-N DNA:
(SEQ ID NO: 1836)
TGCCTGTCATACGAAACCGAGATACTGACAGTAGAATATGGCCTTCTGCCAATCGGG
AAGATTGTGGAGAAACGGATAGAATGCACAGTTTACTCTGTCGATAACAATGGTAA
CATTTATACTCAGCCAGTTGCCCAGTGGCACGACCGGGGAGAGCAGGAAGTATTCG
AATACTGTCTGGAGGATGGAAGTCTCATTAGGGCCACTAAGGACCACAAATTTATG
ACAGTCGATGGCCAGATGCTGCCTATAGACGAAATCTTTGAGCGAGAGTTGGACCTC
ATGCGAGTTGACAACCTTCCTAAT
DnaE Intein-N Protein:
(SEQ ID NO: 1837)
CLSYETEILTVEYGLLPIGKIVEKRIECTVYSVDNNGNIYTQPVAQWHDRGEQEVFEYCL
EDGSLIRATKDHKFMTVDGQMLPIDEIFERELDLMRVDNLPN
DnaE Intein-C DNA:
(SEQ ID NO: 1838)
ATGATCAAGATAGCTACAAGGAAGTATCTTGGCAAACAAAACGTTTATGATATTGG
AGTCGAAAGAGATCACAACTTTGCTCTGAAGAACGGATTCATAGCTTCTAAT
Intein-C:
(SEQ ID NO: 1839)
MIKIATRKYLGKQNVYDIGVERDHNFALKNGFIASN
Cfa-N DNA:
(SEQ ID NO: 1840)
TGCCTGTCTTATGATACCGAGATACTTACCGTTGAATATGGCTTCTTGCCTATTGGAA
AGATTGTCGAAGAGAGAATTGAATGCACAGTATATACTGTAGACAAGAATGGTTTC
GTTTACACACAGCCCATTGCTCAATGGCACAATCGCGGCGAACAAGAAGTATTTGA
GTACTGTCTCGAGGATGGAAGCATCATACGAGCAACTAAAGATCATAAATTCATGA
CCACTGACGGGCAGATGTTGCCAATAGATGAGATATTCGAGCGGGGCTTGGATCTC
AAACAAGTGGATGGATTGCCA
Cfa-N Protein:
(SEQ ID NO: 1841)
CLSYDTEILTVEYGFLPIGKIVEERIECTVYTVDKNGFVYTQPIAQWHNRGEQEVFEYCL
EDGSIIRATKDHKFMTTDGQMLPIDEIFERGLDLKQVDGLP
Cfa-C DNA:
(SEQ ID NO: 1842)
ATGAAGAGGACTGCCGATGGATCAGAGTTTGAATCTCCCAAGAAGAAGAGGAAAGT
AAAGATAATATCTCGAAAAAGTCTTGGTACCCAAAATGTCTATGATATTGGAGTGGA
GAAAGATCACAACTTCCTTCTCAAGAACGGTCTCGTAGCCAGCAAC
Cfa-C Protein:
(SEQ ID NO: 1843)
MKRTADGSEFESPKKKRKVKIISRKSLGTQNVYDIGVEKDHNFLLKNGLVASN

Insert DNAs

In some embodiments, an insert DNA as described herein comprises a nucleic acid sequence that can be integrated into a target DNA molecule, e.g., by a recombinase polypeptide (e.g., a tyrosine recombinase polypeptide), e.g., as described herein. The insert DNA typically is able to bind one or more recombinase polypeptides (e.g., a plurality of copies of a recombinase polypeptide) of the system. In some embodiments the insert DNA comprises a region that is capable of binding a recombinase polypeptide (e.g., a recognition sequence as described herein).

An insert DNA may, in some embodiments, comprise an object sequence for insertion into a target DNA. The object sequence may be coding or non-coding. In some embodiments, the object sequence may contain an open reading frame. In some embodiments the insert DNA comprises a Kozak sequence. In some embodiments the insert DNA comprises an internal ribosome entry site. In some embodiments the insert DNA comprises a self-cleaving peptide such as a T2A or P2A site. In some embodiments the insert DNA comprises a start codon. In some embodiments the insert DNA comprises a splice acceptor site. In some embodiments the insert DNA comprises a splice donor site. In some embodiments the insert DNA comprises a microRNA binding site, e.g., downstream of the stop codon. In some embodiments the insert DNA comprises a polyA tail, e.g., downstream of the stop codon of an open reading frame. In some embodiments the insert DNA comprises one or more exons. In some embodiments the insert DNA comprises one or more introns. In some embodiments the insert DNA comprises a eukaryotic transcriptional terminator. In some embodiments the insert DNA comprises an enhanced translation element or a translation enhancing element. In some embodiments the insert DNA comprises a microRNA sequence, a siRNA sequence, a guide RNA sequence, a piwi RNA sequence. In some embodiments the insert DNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA). In some embodiments, the object sequence may contain a non-coding sequence. For example, the insert DNA may comprise a promoter or enhancer sequence. In some embodiments the insert DNA comprises a tissue specific promoter or enhancer, each of which may be unidirectional or bidirectional. In some embodiments the promoter is an RNA polymerase I promoter, RNA polymerase II promoter, or RNA polymerase III promoter. In some embodiments the promoter comprises a TATA element. In some embodiments the promoter comprises a B recognition element. In some embodiments the promoter has one or more binding sites for transcription factors.

In some embodiments the object sequence of the insert DNA is inserted into a target genome in an endogenous intron. In some embodiments the object sequence of the insert DNA is inserted into a target genome and thereby acts as a new exon. In some embodiments the insertion of the object sequence into the target genome results in replacement of a natural exon or the skipping of a natural exon. In some embodiments the object sequence of the insert DNA is inserted into the target genome in a genomic safe harbor site, such as AAVS1, CCR5, or ROSA26. In some embodiment the object sequence of the insert DNA is added to the genome in an intergenic or intragenic region. In some embodiments the object sequence of the insert DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments the object sequence of the insert DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments the object sequence of the insert DNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments the object sequence of the insert DNA can be, e.g., 1-50 base pairs (e.g., between 1-10, 10-20, 20-30, 30-40, or 40-50 base pairs).

In certain embodiments, an insert DNA can be identified, designed, engineered and constructed to contain sequences altering or specifying the genome function of a target cell or target organism, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, an insert DNA can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails.

The insert DNA may have some homology to the target DNA. In some embodiments the insert DNA has at least 3, 4, 5, 6, 7, 8, 9, 10 or more bases of exact homology to the target DNA or a portion thereof. In some embodiments, the insert DNA has at least 10, 15, 20, 25, 30, 40, 50, 60, 80, 100, 120, 140, 160, 180, 200 or more bases of at least 50%, 60%, 70%, 80%, 85%, 90%, 95%, 97%, 98%, 99% or 100% homology to the target DNA, or a portion thereof.

As an alternative to other methods of delivery described herein, in some embodiments, a nucleic acid (e.g., encoding a recombinase, or a template nucleic acid, or both) delivered to cells is designed as a minicircle, where plasmid backbone sequences not pertaining to Gene Writing™ are removed before administration to cells. Minicircles have been shown to result in higher transfection efficiencies and gene expression as compared to plasmids with backbones containing bacterial parts (e.g., bacterial origin of replication, antibiotic selection cassette) and have been used to improve the efficiency of transposition (Sharma et al. Mol Ther Nucleic Acids 2:E74 (2013)). In some embodiments, the DNA vector encoding the Gene Writer™ polypeptide is delivered as a minicircle. In some embodiments, the DNA vector containing the Gene Writer™ template is delivered as a minicircle. In some embodiments of such alternative means for delivering a nucleic acid, the bacterial parts are flanked by recombination sites, e.g., attP/attB, loxP, FRT sites. In some embodiments, the addition of a cognate recombinase results in intramolecular recombination and excision of the bacterial parts. In some embodiments, the recombinase sites are recognized by phiC31 recombinase. In some embodiments, the recombinase sites are recognized by Cre recombinase. In some embodiments, the recombinase sites are recognized by FLP recombinase. In some embodiments, minicircles are generated in a bacterial production strain, e.g., an E. coli strain stably expressing inducible minicircle assembling enzymes, e.g., a producer strain as according to Kay et al. Nat Biotechnol 28(12):1287-1289 (2010). Minicircle DNA vector preparations and methods of production are described in U.S. Pat. No. 9,233,174, incorporated herein by reference in its entirety.

In addition to plasmid DNA, minicircles can be generated by excising the desired construct, e.g., recombinase expression cassette or therapeutic expression cassette, from a viral backbone, e.g., an AAV vector. Previously, it has been shown that excision and circularization of the insert DNA sequence from a viral backbone may be important for transposase-mediated integration efficiency (Yant et al. Nat Biotechnol 20(10):999-1005 (2002)). In some embodiments, minicircles are first formulated and then delivered to target cells. In other embodiments, minicircles are formed from a DNA vector (e.g., plasmid DNA, rAAV, scAAV, ceDNA, doggybone DNA) intracellularly by co-delivery of a recombinase, resulting in excision and circularization of the recombinase recognition site-flanked nucleic acid, e.g., a nucleic acid encoding the Gene Writer™ polypeptide, or DNA template, or both. In some embodiments, the same recombinase is used for a first excision event (e.g., intramolecular recombination) and a second integration (e.g., target site integration) event. In some embodiments, the recombination site on an excised circular DNA (e.g., after a first recombination event, e.g., intramolecular recombination) is used as the template recognition site for a second recombination (e.g., target site integration) event.

Linkers

In some embodiments, domains of the compositions and systems described herein (e.g., the recombinase domain and/or DNA recognition domains of a recombinase polypeptide, e.g., as described herein) may be joined by a linker. A composition described herein comprising a linker element has the general form S1-L-S2, wherein S1 and S2 may be the same or different and represent two domain moieties (e.g., each a polypeptide or nucleic acid domain) associated with one another by the linker. In some embodiments, a linker may connect two polypeptides. In some embodiments, a linker may connect two nucleic acid molecules. In some embodiments, a linker may connect a polypeptide and a nucleic acid molecule. A linker may be a chemical bond, e.g., one or more covalent bonds or non-covalent bonds. A linker may be flexible, rigid, and/or cleavable. In some embodiments, the linker is a peptide linker. Generally, a peptide linker is at least 2, 3, 4, 5, 6, 7, 8, 9, 10 or more amino acids in length, e.g., 2-50 amino acids in length, 2-30 amino acids in length.

The most commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the other moieties. Examples of such linkers include those having the structure [GGS]^{≥1} or [GGGS]^{≥1} (SEQ ID NO: 1844). Rigid linkers are useful to keep a fixed distance between domains and to maintain their independent functions. Rigid linkers may also be useful when a spatial separation of the domains is critical to preserve the stability or bioactivity of one or more components in the agent. Rigid linkers may have an alpha helix-structure or Pro-rich sequence, (XP)n, with X designating any amino acid, preferably Ala, Lys, or Glu. Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in compositions described herein may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

In some embodiments the amino acid linkers are (or are homologous to) the endogenous amino acids that exist between such domains in a native polypeptide. In some embodiments the endogenous amino acids that exist between such domains are substituted but the length is unchanged from the natural length. In some embodiments, additional amino acid residues are added to the naturally existing amino acid residues between domains.

In some embodiments, the amino acid linkers are designed computationally or screened to maximize protein function (Anad et al., FEBS Letters, 587:19, 2013).

### Genomic Safe Harbor Sites

In some embodiments, a Gene Writer targets a genomic safe harbor site (e.g., directs insertion of a heterologous object sequence into a position having a safe harbor score of at least 3, 4, 5, 6, 7, or 8). In some embodiments the genomic safe harbor site is a Natural Harbor™ site. In some embodiments, a Natural Harbor™ site is derived from the native target of a mobile genetic element, e.g., a recombinase, transposon, retrotransposon, or retrovirus. The native targets of mobile elements may serve as ideal locations for genomic integration given their evolutionary selection. In some embodiments the Natural Harbor™ site is ribosomal DNA (rDNA). In some embodiments the Natural Harbor™ site is 5S rDNA, 18S rDNA, 5.8S rDNA, or 28S rDNA. In some embodiments the Natural Harbor™ site is the Mutsu site in 5S rDNA. In some embodiments the Natural Harbor™ site is the R2 site, the R5 site, the R6 site, the R4 site, the R1 site, the R9 site, or the RT site in 28S rDNA. In some embodiments the Natural Harbor™ site is the R8 site or the R7 site in 18S rDNA. In some embodiments the Natural Harbor™ site is DNA encoding transfer RNA (tRNA). In some embodiments the Natural Harbor™ site is DNA encoding tRNA-Asp or tRNA-Glu. In some embodiments the Natural Harbor™ site is DNA encoding spliceosomal RNA. In some embodiments the Natural Harbor™ site is DNA encoding small nuclear RNA (snRNA) such as U2 snRNA.

Thus, in some aspects, the present disclosure provides a method comprising comprises using a GeneWriter system described herein to insert a heterologous object sequence into a Natural Harbor™ site. In some embodiments, the Natural Harbor™ site is a site described in Table 4 below. In some embodiments, the heterologous object sequence is inserted within 20, 50, 100, 150, 200, 250, 500, or 1000 base pairs of the Natural Harbor™ site. In some embodiments, the heterologous object sequence is inserted within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of the Natural Harbor™ site. In some embodiments, the heterologous object sequence is inserted into a site having at least 70%, 75%, 80%, 85%, 90%, 95%, 96%, 97%, 98%, or 99% identity to a sequence shown in Table 4. In some embodiments, the heterologous object sequence is inserted within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of a site having at least 70%, 75%, 80%, 85%, 90%, 95%, 96%, 97%, 98%, or 99% identity to a sequence shown in Table 4. In some embodiments, the heterologous object sequence is inserted within a gene indicated in Column 5 of Table 4, or within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of the gene.

TABLE 4
Natural Harbor ™ sites. Column 1 indicates a retrotransposon that inserts into the
Natural Harbor ™ site. Column 2 indicates the gene at the Natural Harbor™ site. Columns 3
and 4 show exemplary human genome sequence 5’ and 3’ of the insertion site (for example, 250
bp). Columns 5 and 6 list the example gene symbol and corresponding Gene ID.

| | | | | Example | |
|---|---|---|---|---|---|
| Target | Target | | | Gene | Example |
| Site | Gene | 5' flanking sequence | 3' flanking sequence | Symbol | Gene ID |
| R2 | 28S | CCGGTCCCCCCCGC | GTAGCCAAATGCCT | RNA28SN1 | 106632264 |
| | rDNA | CGGGTCCGCCCCCG | CGTCATCTAATTAG | | |
| | | GGGCCGCGGTTCCG | TGACGCGCATGAAT | | |
| | | CGCGGCGCCTCGCC | GGATGAACGAGATT | | |
| | | TCGGCCGGCGCCTA | CCCACTGTCCCTAC | | |
| | | GCAGCCGACTTAGA | CTACTATCCAGCGA | | |
| | | ACTGGTGCGGACCA | AACCACAGCCAAG | | |
| | | GGGGAATCCGACTG | GGAACGGGCTTGGC | | |
| | | TTTAATTAAAACAA | GGAATCAGCGGGG | | |
| | | AGCATCGCGAAGGC | AAAGAAGACCCTGT | | |
| | | CCGCGGCGGGTGTT | TGAGCTTGACTCTA | | |
| | | GACGCGATGTGATT | GTCTGGCACGGTGA | | |
| | | TCTGCCCAGTGCTC | AGAGACATGAGAG | | |
| | | TGAATGTCAAAGTG | GTGTAGAATAAGTG | | |
| | | AAGAAATTCAATGA | GGAGGCCCCCGGCG | | |
| | | AGCGCGGGTAAAC | CCCCCCCGGTGTCC | | |
| | | GGCGGGAGTAACTA | CCGCGAGGGGCCCG | | |
| | | TGACTCTCTTAAG | GGGCGGGGTCCGCC | | |
| | | (SEQ ID NO: 1845) | G (SEQ ID NO: 1856) | | |
| R4 | 28S | GCGGTTCCGCGCGG | CGCATGAATGGATG | RNA28SN1 | 106632264 |
| | rDNA | CGCCTCGCCTCGGC | AACGAGATTCCCAC | | |
| | | CGGCGCCTAGCAGC | TGTCCCTACCTACT | | |
| | | CGACTTAGAACTGG | ATCCAGCGAAACCA | | |
| | | TGCGGACCAGGGG | CAGCCAAGGGAAC | | |
| | | AATCCGACTGTTTA | GGGCTTGGCGGAAT | | |
| | | ATTAAAACAAAGCA | CAGCGGGGAAAGA | | |
| | | TCGCGAAGGCCCGC | AGACCCTGTTGAGC | | |
| | | GGCGGGTGTTGACG | TTGACTCTAGTCTG | | |
| | | CGATGTGATTTCTG | GCACGGTGAAGAG | | |
| | | CCCAGTGCTCTGAA | ACATGAGAGGTGTA | | |
| | | TGTCAAAGTGAAGA | GAATAAGTGGGAG | | |
| | | AATTCAATGAAGCG | GCCCCCGGCGCCCC | | |
| | | CGGGTAAACGGCG | CCCGGTGTCCCCGC | | |
| | | GGAGTAACTATGAC | GAGGGGCCCGGGG | | |
| | | TCTCTTAAGGTAGC | CGGGGTCCGCCGGC | | |
| | | CAAATGCCTCGTCA | CCTGCGGGCCGCCG | | |
| | | TCTAATTAGTGACG | GTGAAATACCACTA | | |
| | | (SEQ ID NO: 1846) | CTC (SEQ ID NO: | | |
| | | | 1857) | | |
| R5 | 28S | TCCCCCCCGCCGGG | CCAAATGCCTCGTC | RNA28SN1 | 106632264 |
| | rDNA | TCCGCCCCCGGGGC | ATCTAATTAGTGAC | | |
| | | CGCGGTTCCGCGCG | GCGCATGAATGGAT | | |
| | | GCGCCTCGCCTCGG | GAACGAGATTCCCA | | |
| | | CCGGCGCCTAGCAG | CTGTCCCTACCTAC | | |
| | | CCGACTTAGAACTG | TATCCAGCGAAACC | | |
| | | GTGCGGACCAGGG | ACAGCCAAGGGAA | | |
| | | GAATCCGACTGTTT | CGGGCTTGGCGGAA | | |
| | | AATTAAAACAAAGC | TCAGCGGGGAAAG | | |
| | | ATCGCGAAGGCCCG | AAGACCCTGTTGAG | | |
| | | CGGCGGGTGTTGAC | CTTGACTCTAGTCT | | |
| | | GCGATGTGATTTCT | GGCACGGTGAAGA | | |
| | | GCCCAGTGCTCTGA | GACATGAGAGGTGT | | |
| | | ATGTCAAAGTGAAG | AGAATAAGTGGGA | | |
| | | AAATTCAATGAAGC | GGCCCCCGGCGCCC | | |
| | | GCGGGTAAACGGC | CCCCGGTGTCCCCG | | |
| | | GGGAGTAACTATGA | CGAGGGGCCCGGG | | |
| | | CTCTCTTAAGGTAG | GCGGGGTCCGCCGG | | |
| | | (SEQ ID NO: 1847) | CCC (SEQ ID NO: | | |
| | | | 1858) | | |
| R9 | 28S | CGGCGCGCTCGCCG | TAGCTGGTTCCCTC | RNA28SN1 | 106632264 |
| | rDNA | GCCGAGGTGGGATC | CGAAGTTTCCCTCA | | |
| | | CCGAGGCCTCTCCA | GGATAGCTGGCGCT | | |
| | | GTCCGCCGAGGGCG | CTCGCAGACCCGAC | | |
| | | CACCACCGGCCCGT | GCACCCCCGCCACG | | |
| | | CTCGCCCGCCGCGC | CAGTTTTATCCGGT | | |
| | | CGGGGAGGTGGAG | AAAGCGAATGATTA | | |
| | | CACGAGCGCACGTG | GAGGTCTTGGGGCC | | |
| | | TTAGGACCCGAAAG | GAAACGATCTCAAC | | |
| | | ATGGTGAACTATGC | CTATTCTCAAACTT | | |
| | | CTGGGCAGGGCGA | TAAATGGGTAAGAA | | |
| | | AGCCAGAGGAAAC | GCCCGGCTCGCTGG | | |
| | | TCTGGTGGAGGTCC | CGTGGAGCCGGGCG | | |
| | | GTAGCGGTCCTGAC | TGGAATGCGAGTGC | | |
| | | GTGCAAATCGGTCG | CTAGTGGGCCACTT | | |
| | | TCCGACCTGGGTAT | TTGGTAAGCAGAAC | | |
| | | AGGGGCGAAAGAC | TGGCGCTGCGGGAT | | |
| | | TAATCGAACCATCT | GAACCGAACGCC | | |
| | | AG (SEQ ID NO: 1848) | (SEQ ID NO: 1859) | | |
| R8 | 18S | GCATTCGTATTGCG | TGAAACTTAAAGGA | RNA18SN1 | 106631781 |
| | rDNA | CCGCTAGAGGTGAA | ATTGACGGAAGGGC | | |
| | | ATTCTTGGACCGGC | ACCACCAGGAGTGG | | |
| | | GCAAGACGGACCA | AGCCTGCGGCTTAA | | |
| | | GAGCGAAAGCATTT | TTTGACTCAACACG | | |
| | | GCCAAGAATGTTTT | GGAAACCTCACCCG | | |
| | | CATTAATCAAGAAC | GCCCGGACACGGAC | | |
| | | GAAAGTCGGAGGTT | AGGATTGACAGATT | | |
| | | CGAAGACGATCAG | GATAGCTCTTTCTC | | |
| | | ATACCGTCGTAGTT | GATTCCGTGGGTGG | | |
| | | CCGACCATAAACGA | TGGTGCATGGCCGT | | |
| | | TGCCGACCGGCGAT | TCTTAGTTGGTGGA | | |
| | | GCGGCGGCGTTATT | GCGATTTGTCTGGT | | |
| | | CCCATGACCCGCCG | TAATTCCGATAACG | | |
| | | GGCAGCTTCCGGGA | AACGAGACTCTGGC | | |
| | | AACCAAAGTCTTTG | ATGCTAACTAGTTA | | |
| | | GGTTCCGGGGGGAG | CGCGACCCCCGAGC | | |
| | | TATGGTTGCAAAGC | GGTCGGCGTCCC | | |
| | | (SEQ ID NO: 1849) | (SEQ ID NO: 1860) | | |
| R4- | tRNA- | | | TRD- | 100189207 |
| 2_SRa | Asp | | | GTC1-1 | |
| LIN25_ | tRNA- | | | TRE- | 100189384 |
| SM | Glu | | | CTC1-1 | |
| R1 | 28S | TAGCAGCCGACTTA | ACCTACTATCCAGC | RNA28SN1 | 106632264 |
| | rDNA | GAACTGGTGCGGAC | GAAACCACAGCCA | | |
| | | CAGGGGAATCCGAC | AGGGAACGGGCTTG | | |
| | | TGTTTAATTAAAAC | GCGGAATCAGCGG | | |
| | | AAAGCATCGCGAA | GGAAAGAAGACCC | | |
| | | GGCCCGCGGCGGGT | TGTTGAGCTTGACT | | |
| | | GTTGACGCGATGTG | CTAGTCTGGCACGG | | |
| | | ATTTCTGCCCAGTG | TGAAGAGACATGA | | |
| | | CTCTGAATGTCAAA | GAGGTGTAGAATAA | | |
| | | GTGAAGAAATTCAA | GTGGGAGGCCCCCG | | |
| | | TGAAGCGCGGGTAA | GCGCCCCCCCGGTG | | |
| | | ACGGCGGGAGTAA | TCCCCGCGAGGGGC | | |
| | | CTATGACTCTCTTA | CCGGGGCGGGGTCC | | |
| | | AGGTAGCCAAATGC | GCCGGCCCTGCGGG | | |
| | | CTCGTCATCTAATT | CCGCCGGTGAAATA | | |
| | | AGTGACGCGCATGA | CCACTACTCTGATC | | |
| | | ATGGATGAACGAG | GTTTTTTCACTGAC | | |
| | | ATTCCCACTGTCCC | CCGGTGAGGCGGG | | |
| | | T (SEQ ID NO: 1850) | GGG (SEQ ID NO: | | |
| | | | 1861) | | |
| R6 | 28S | CCCCCCGCCGGGTC | AAATGCCTCGTCAT | RNA28SN1 | 106632264 |
| | rDNA | CGCCCCCGGGGCCG | CTAATTAGTGACGC | | |
| | | CGGTTCCGCGCGGC | GCATGAATGGATGA | | |
| | | GCCTCGCCTCGGCC | ACGAGATTCCCACT | | |
| | | GGCGCCTAGCAGCC | GTCCCTACCTACTA | | |
| | | GACTTAGAACTGGT | TCCAGCGAAACCAC | | |
| | | GCGGACCAGGGGA | AGCCAAGGGAACG | | |
| | | ATCCGACTGTTTAA | GGCTTGGCGGAATC | | |
| | | TTAAAACAAAGCAT | AGCGGGGAAAGAA | | |
| | | CGCGAAGGCCCGCG | GACCCTGTTGAGCT | | |
| | | GCGGGTGTTGACGC | TGACTCTAGTCTGG | | |
| | | GATGTGATTTCTGC | CACGGTGAAGAGA | | |
| | | CCAGTGCTCTGAAT | CATGAGAGGTGTAG | | |
| | | GTCAAAGTGAAGA | AATAAGTGGGAGG | | |
| | | AATTCAATGAAGCG | CCCCCGGCGCCCCC | | |
| | | CGGGTAAACGGCG | CCGGTGTCCCCGCG | | |
| | | GGAGTAACTATGAC | AGGGGCCCGGGGC | | |
| | | TCTCTTAAGGTAGC | GGGGTCCGCCGGCC | | |
| | | C (SEQ ID NO: 1851) | CTG (SEQ ID NO: | | |
| | | | 1862) | | |
| R7 | 18S | GCGCAAGACGGAC | GGAGCCTGCGGCTT | RNA18SN1 | 106631781 |
| | rDNA | CAGAGCGAAAGCA | AATTTGACTCAACA | | |
| | | TTTGCCAAGAATGT | CGGGAAACCTCACC | | |
| | | TTTCATTAATCAAG | CGGCCCGGACACGG | | |
| | | AACGAAAGTCGGA | ACAGGATTGACAGA | | |
| | | GGTTCGAAGACGAT | TTGATAGCTCTTTCT | | |
| | | CAGATACCGTCGTA | CGATTCCGTGGGTG | | |
| | | GTTCCGACCATAAA | GTGGTGCATGGCCG | | |
| | | CGATGCCGACCGGC | TTCTTAGTTGGTGG | | |
| | | GATGCGGCGGCGTT | AGCGATTTGTCTGG | | |
| | | ATTCCCATGACCCG | TTAATTCCGATAAC | | |
| | | CCGGGCAGCTTCCG | GAACGAGACTCTGG | | |
| | | GGAAACCAAAGTCT | CATGCTAACTAGTT | | |
| | | TTGGGTTCCGGGGG | ACGCGACCCCCGAG | | |
| | | GAGTATGGTTGCAA | CGGTCGGCGTCCCC | | |
| | | AGCTGAAACTTAAA | CAACTTCTTAGAGG | | |
| | | GGAATTGACGGAA | GACAAGTGGCGTTC | | |
| | | GGGCACCACCAGG | AGCCACCCGAG | | |
| | | AGT (SEQ ID NO: | (SEQ ID NO: 1863) | | |
| | | 1852) | | | |
| RT | 28S | GGCCGGGCGCGACC | AACTGGCTTGTGGC | RNA28SN1 | 106632264 |
| | rDNA | CGCTCCGGGGACAG | GGCCAAGCGTTCAT | | |
| | | TGCCAGGTGGGGAG | AGCGACGTCGCTTT | | |
| | | TTTGACTGGGGCGG | TTGATCCTTCGATG | | |
| | | TACACCTGTCAAAC | TCGGCTCTTCCTAT | | |
| | | GGTAACGCAGGTGT | CATTGTGAAGCAGA | | |
| | | CCTAAGGCGAGCTC | ATTCACCAAGCGTT | | |
| | | AGGGAGGACAGAA | GGATTGTTCACCCA | | |
| | | ACCTCCCGTGGAGC | CTAATAGGGAACGT | | |
| | | AGAAGGGCAAAAG | GAGCTGGGTTTAGA | | |
| | | CTCGCTTGATCTTG | CCGTCGTGAGACAG | | |
| | | ATTTTCAGTACGAA | GTTAGTTTTACCCT | | |
| | | TACAGACCGTGAAA | ACTGATGATGTGTT | | |
| | | GCGGGGCCTCACGA | GTTGCCATGGTAAT | | |
| | | TCCTTCTGACCTTTT | CCTGCTCAGTACGA | | |
| | | GGGTTTTAAGCAGG | GAGGAACCGCAGG | | |
| | | AGGTGTCAGAAAA | TTCAGACATTTGGT | | |
| | | GTTACCACAGGGAT | GTATGTGCTTGGC | | |
| | | (SEQ ID NO: 1853) | (SEQ ID NO: 1864) | | |
| Mutsu | 5S | GTCTACGGCCATAC | TGAACGCGCCCGAT | RNA5S1 | 100169751 |
| | rDNA | CACCC (SEQ ID NO: | CTCGTCTGATCTCG | | |
| | | 1854) | GAAGCTAAGCAGG | | |
| | | | GTCGGGCCTGGTTA | | |
| | | | GTACTTGGATGGGA | | |
| | | | GACCGCCTGGGAAT | | |
| | | | ACCGGGTGCTGTAG | | |
| | | | GCTTT (SEQ ID NO: | | |
| | | | 1865) | | |
| Utopia/ | U2 | ATCGCTTCTCGGCC | TCTGTTCTTATCAGT | RNU2-1 | 6066 |
| Keno | snRNA | TTTTGGCTAAGATC | TTAATATCTGATAC | | |
| | | AAGTGTAGTA (SEQ | GTCCTCTATCCGAG | | |
| | | ID NO: 1855) | GACAATATATTAAA | | |
| | | | TGGATTTTTGGAGC | | |
| | | | AGGGAGATGGAAT | | |
| | | | AGGAGCTTGCTCCG | | |
| | | | TCCACTCCACGCAT | | |
| | | | CGACCTGGTATTGC | | |
| | | | AGTACCTCCAGGAA | | |
| | | | CGGTGCACCC (SEQ | | |
| | | | ID NO: 1866) | | |

### Additional Functional Characteristics for Gene Writers™

A Gene Writer as described herein may, in some instances, be characterized by one or more functional measurements or characteristics. In some embodiments, the DNA binding domain (e.g., target binding domain) has one or more of the functional characteristics described below. In some embodiments, the template binding domain has one or more of the functional characteristics described below. In some embodiments, the template (e.g., template DNA) has one or more of the functional characteristics described below. In some embodiments, the target site altered by the Gene Writer has one or more of the functional characteristics described below following alteration by the Gene Writer.

Gene Writer Polypeptide

DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from the Cre recombinase of bacteriophage P1. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 pM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

Template Binding Domain

In some embodiments, the template binding domain is capable of binding to a template DNA with greater affinity than a reference DNA binding domain. In some embodiments, the reference DNA binding domain is a DNA binding domain from the Cre recombinase of bacteriophage P1. In some embodiments, the template binding domain is capable of binding to a template DNA with an affinity between 100 pM-10 nM (e.g., between 100 pM-1 nM or 1 nM-10 nM). In some embodiments, the affinity of a DNA binding domain for its template DNA is measured in vitro, e.g., by thermophoresis, e.g., as described in Asmari et al. Methods 146:107-119 (2018) (incorporated by reference herein in its entirety). In some embodiments, the affinity of a DNA binding domain for its template DNA is measured in cells (e.g., by FRET or ChIP-Seq).

In some embodiments, the DNA binding domain is associated with the template DNA in vitro with at least 50% template DNA bound in the presence of 10 nM competitor DNA, e.g., as described in Yant et al. Mol Cell Biol 24(20):9239-9247 (2004) (incorporated by reference herein in its entirety). In some embodiments, the DNA binding domain is associated with the template DNA in cells (e.g., in HEK293T cells) at a frequency at least about 5-fold or 10-fold higher than with a scrambled DNA. In some embodiments, the frequency of association between the DNA binding domain and the template DNA or scrambled DNA is measured by ChIP-seq, e.g., as described in He and Pu (2010), supra.

Target Site

In some embodiments, after Gene Writing, the target site surrounding the integrated sequence contains a limited number of insertions or deletions, for example, in less than about 50% or 10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple insertion events, e.g., head-to-tail or head-to-head duplications, e.g., as determined by long-read amplicon sequencing of the target site, e.g., as described in Karst et al. (2020), supra. In some embodiments, the target site contains an integrated sequence corresponding to the template DNA. In some embodiments, the target site contains a completely integrated template molecule. In some embodiments, the target site contains components of the vector DNA, e.g., AAV ITRs. In some embodiments, e.g., when a template DNA is first excised from a viral vector by a first recombination event prior to integration, the target site does not contain insertions resulting from non-template DNA, e.g., endogenous or vector DNA, e.g., AAV ITRs, in more than about 1% or 10% of events, e.g., as determined by long-read amplicon sequencing of the target site, e.g., as described in Karst et al. (2020), supra. In some embodiments, the target site contains the integrated sequence corresponding to the template DNA.

In some embodiments, a Gene Writer described herein is capable of site-specific editing of target DNA, e.g., insertion of template DNA into a target DNA. In some embodiments, a site-specific Gene Writer is capable of generating an edit, e.g., an insertion, that is present at the target site with a higher frequency than any other site in the genome. In some embodiments, a site-specific Gene Writer is capable of generating an edit, e.g., an insertion in a target site at a frequency of at least 2, 3, 4, 5, 10, 50, 100, or 1000-fold that of the frequency at all other sites in the human genome. In some embodiments, the location of integration sites is determined by unidirectional sequencing. The incorporation of unique molecular identifiers (UMI) in the adapters or primers used in library preparation allows the quantification of discrete insertion events, which can be compared between on-target insertions and all other insertions to determine the preference for the defined target site.

In some embodiments, a Gene Writing system is used to edit a target DNA sequence that is present at a single location in the human genome. In some embodiments, a Gene Writing system is used to edit a target DNA sequence that is present at a single location in the human genome on a single homologous chromosome, e.g., is haplotype-specific. In some embodiments, a Gene Writing system is used to edit a target DNA sequence that is present at a single location in the human genome on two homologous chromosomes. In some embodiments, a Gene Writing system is used to edit a target DNA sequence that is present in multiple locations in the genome, e.g., at least 2, 3, 4, 5, 10, 20, 50, 100, 200, 500, 1000, 5000, 10000, 100000, 200000, 500000, 1000000 (e.g., Alu elements) locations in the genome.

In some embodiments, a Gene Writer system is able to edit a genome without introducing undesirable mutations. In some embodiments, a Gene Writer system is able to edit a genome by inserting a template, e.g., template DNA, into the genome. In some embodiments, the resulting modification in the genome contains minimal mutations relative to the template DNA sequence. In some embodiments, the average error rate of genomic insertions relative to the template DNA is less than 10⁻⁴, 10⁻⁵, or 10⁻⁶ mutations per nucleotide. In some embodiments, the number of mutations relative to a template DNA that is introduced into a target cell averages less than 1, 2, 3, 4, 5, 10, 20, 30, 40, 50, 60, 70, 80, 90, or 100 nucleotides per genome. In some embodiments, the error rate of insertions in a target genome is determined by long-read amplicon sequencing across known target sites, e.g., as described in Karst et al. (2020), supra, and comparing to the template DNA sequence. In some embodiments, errors enumerated by this method include nucleotide substitutions relative to the template sequence. In some embodiments, errors enumerated by this method include nucleotide deletions relative to the template sequence. In some embodiments, errors enumerated by this method include nucleotide insertions relative to the template sequence. In some embodiments, errors enumerated by this method include a combination of one or more of nucleotide substitutions, deletions, or insertions relative to the template sequence.

Efficiency of integration events can be used as a measure of editing of target sites or target cells by a Gene Writer system. In some embodiments, a Gene Writer system described herein is capable of integrating a heterologous object sequence in a fraction of target sites or target cells. In some embodiments, a Gene Writer system is capable of editing at least 1%, 2%, 5%, 10%, 20%, 30%, 40%, 50%, 60%, 70%, 80%, 90%, 95%, 96%, 97%, 98%, 99%, 99.5%, 99.9% or 100% of target loci as measured by the detection of the edit when amplifying across the target and analyzing with long-read amplicon sequencing, e.g., as described in Karst et al. (2020). In some embodiments, a Gene Writer system is capable of editing cells at an average copy number of at least 0.1, e.g., at least 0.1, 0.5, 1, 2, 3, 4, 5, 10, or 100 copies per genome as normalized to a reference gene, e.g., RPP30, across a population of cells, e.g., as determined by ddPCR with transgene-specific primer-probe sets, e.g., as according to the methods in Lin et al. Hum Gene Ther Methods 27(5):197-208 (2016).

In some embodiments, the copy number per cell is analyzed by single-cell ddPCR (sc-ddPCR), e.g., as according to the methods of Igarashi et al. Mol Ther Methods Clin Dev 6:8-16 (2017), incorporated herein by reference in its entirety. In some embodiments, at least 1%, e.g., at least 1%, 2%, 5%, 10%, 20%, 30%, 40%, 50%, 60%, 70%, 80%, 90%, 95%, 96%, 97%, 98%, 99%, 99.5%, 99.9% or 100%, of target cells are positive for integration as assessed by sc-ddPCR using transgene-specific primer-probe sets. In some embodiments, the average copy number is at least 0.1, e.g., at least 0.1, 0.5, 1, 2, 3, 4, 5, 10, or 100 copies per cell as measured by sc-ddPCR using transgene-specific primer-probe sets.

Additional Gene Writer Characteristics

In some embodiments, the Gene Writer system may result in complete writing without requiring endogenous host factors. In some embodiments, the system may result in complete writing without the need for DNA repair. In some embodiments, the system may result in complete writing without eliciting a DNA damage response.

In some embodiments, the system does not require DNA repair by the NHEJ pathway, homologous recombination repair pathway, base excision repair pathway, or any combination thereof. Participation by a DNA repair pathway can be assayed, for example, via the application of DNA repair pathway inhibitors or DNA repair pathway deficient cell lines. For example, when applying DNA repair pathway inhibitors, PrestoBlue cell viability assay can be performed first to determine the toxicity of the inhibitors and whether any normalization should be applied. SCR7 is an inhibitor for NHEJ, which can be applied at a series of dilutions during Gene Writer™ delivery. PARP protein is a nuclear enzyme that binds as homodimers to both single- and double-strand breaks. Thus, its inhibitors can be used in the test of relevant DNA repair pathways, including homologous recombination repair pathway and base excision repair pathway. The experiment procedure is the same with that of SCR7. Cell lines with deficient core proteins of nucleotide excision repair (NER) pathway can be used to test the effect of NER on Gene Writing™. After the delivery of the Gene Writer™ system into the cell, ddPCR can used to evaluate the insertion of a heterologous object sequence in the context of inhibition of DNA repair pathways. Sequencing analysis can also be performed to evaluate whether certain DNA repair pathways play a role. In some embodiments, Gene Writing™ into the genome is not decreased by the knockdown of a DNA repair pathway described herein. In some embodiments, Gene Writing™ into the genome is not decreased by more than 50% by the knockdown of the DNA repair pathway.

### Evolved Variants of Gene Writers

In some embodiments, the invention provides evolved variants of Gene Writers. Evolved variants can, in some embodiments, be produced by mutagenizing a reference Gene Writer, or one of the fragments or domains comprised therein. In some embodiments, one or more of the domains (e.g., the catalytic or DNA binding domain (e.g., target binding domain or template binding domain), including, for example, sequence-guided DNA binding elements) is evolved, One or more of such evolved variant domains can, in some embodiments, be evolved alone or together with other domains. An evolved variant domain or domains may, in some embodiments, be combined with unevolved cognate component(s) or evolved variants of the cognate component(s). e.g., which may have been evolved in either a parallel or serial manner.

In some embodiments, the process of mutagenizing a reference Gene Writer, or fragment or domain thereof, comprises mutagenizing the reference Gene Writer or fragment or domain thereof. In embodiments, the mutagenesis comprises a continuous evolution method (e.g., PACE) or non-continuous evolution method (e.g., PANCE). e.g., as described herein. In some embodiments, the evolved Gene Writer, or a fragment or domain thereof (e.g., a DNA binding domain, e.g., a target binding domain or a template binding domain), comprises one or more amino acid variations introduced into its amino acid sequence relative to the amino acid sequence of the reference Gene Writer, or fragment or domain thereof. In embodiments, amino acid sequence variations may include one or more mutated residues (e.g., conservative substitutions, non-conservative substitutions, or a combination thereof) within the amino acid sequence of a reference Gene Writer, e.g., as a result of a change in the nucleotide sequence encoding the gene writer that results in, e.g., a change in the codon at any particular position in the coding sequence, the deletion of one or more amino acids (e.g., a truncated protein), the insertion of one or more amino acids, or any combination of the foregoing. The evolved variant Gene Writer may include variants in one or more components or domains of the Gene Writer (e.g., variants introduced into a catalytic domain, DNA binding domain, or combinations thereof).

In some aspects, the invention provides Gene Writers, systems, kits, and methods using or comprising an evolved variant of a Gene Writer, e.g., employs an evolved variant of a Gene Writer or a Gene Writer produced or producible by PACE or PANCE. In embodiments, the unevolved reference Gene Writer is a Gene Writer as disclosed herein.

The term “phage-assisted continuous evolution (PACE),” as used herein, generally refers to continuous evolution that employs phage as viral vectors. Examples of PACE technology have been described, for example, in International PCT Application No. PCT/US 2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; international PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANC II)” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat Chem Biol. 13(12): 1261-1266 (2017), incorporated herein by reference in its entirety, Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to Gene Writers may be readily appreciated by the skilled artisan by reference to, inter alia, the foregoing references. Additional exemplary methods for directing continuous evolution of genome-modifying proteins or systems, e.g., in a population of host cells e.g., using phage particles, can be applied to generate evolved variants of Gene Writers. or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017: U.S. Pat. No. 9,394,537, issued Jul. 19, 2016: International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant Gene Writer, of a fragment or domain thereof, comprises: (a) contacting a population of host cells with a population of viral vectors comprising the gene of interest (the starting Gene Writer or fragment or domain thereof), wherein: (1) the host cell is amenable to infection by the viral vector; (2) the host cell expresses viral genes required for the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (b) contacting the host cells with a Mutagen, using host cells with mutations that elevate mutation rate (e.g., either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter), or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (dl) isolating a mutated version of the viral vector, encoding an evolved gene product (e.g., an evolved variant Gene Writer, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gIII but otherwise comprise gI, gII, gIV, gV, gVI, gVII, gVIII, gIX, and a gX. In some embodiments, the generation of infectious VSV particles involves the envelope protein VSV-G. Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500 at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml about 5-10⁷ cells/ml about 10⁸ cells/ml, about 5-10⁸ cells/ml, about 10⁹ cells/ml about 5·10⁹ cells/ml about 10¹⁰ cells/ml, or about 5·10¹⁰ cells/ml.

### Nucleic Acids

### Promoters

In some embodiments, one or more promoter or enhancer elements are operably linked to a nucleic acid encoding a Gene Writer polypeptide or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbomylase promoter and enhancer may be used to control expression of the ornithine transcarbomylase gene in a system or method provided by the invention for correcting ornithine transcarbomylase deficiencies. In some embodiments, the promoter is a promoter of Table 33 or a functional fragment or variant thereof.

Exemplary tissue specific promoters that are commercially available can be found, for example, at a uniform resource locator (e.g., invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR. In some embodiments, the 5° UTR comprises an intron. In other embodiments, these include composite promoters, which combine promoter elements of different origins or were generated by assembling a distal enhancer with a minimal promoter of the same origin. In some embodiments, a tissue-specific expression-control sequence(s) comprises one or more of the sequences in Table 2 or Table 3 of PCT Publication No. WO2020014209 (incorporated herein by reference in its entirety).

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (http://epd.epfl.ch//index.php).

TABLE 5
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 | pancreatic |
| promoter | acinar cells |
| Endoglin promoter | endothelial cells |
| fibronectin | differentiating cells, |
| promoter | healing tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs 1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb | Liver |
| promoter | |
| SV40/bAlb | Liver |
| promoter | |
| SV40/Cd3 | Leukocytes and platelets |
| promoter | |
| SV40/CD45 | hematopoeitic cells |
| promoter | |
| NSE/RU5′ | Mature Neurons |
| promoter | |

TABLE 6
Additional exemplary cell or tissue-specific promoters

| Promoter | Gene Description | Gene Specificity |
|---|---|---|
| APOA2 | Apolipoprotein A-II | Hepatocytes (from hepatocyte |
| | | progenitors) |
| SERPINA | Serpin peptidase inhibitor, clade A | Hepatocytes |
| 1 (hAAT) | (alpha-1 | (from definitive endoderm |
| | antiproteinase, antitrypsin), member 1 | stage) |
| | (also named alpha 1 anti-tryps in) | |
| CYP3A | Cytochrome P450, family 3, | Mature Hepatocytes |
| | subfamily A, polypeptide | |
| MIR122 | MicroRNA 122 | Hepatocytes |
| | | (from early stage embryonic |
| | | liver cells) |
| | | and endoderm |

Pancreatic specific promoters

| | | |
|---|---|---|
| INS | Insulin | Pancreatic beta cells |
| | | (from definitive endoderm stage) |
| IRS2 | Insulin receptor substrate 2 | Pancreatic beta cells |
| Pdx1 | Pancreatic and duodenal | Pancreas |
| | homeobox 1 | (from definitive endoderm stage) |
| A1x3 | Aristaless-like homeobox 3 | Pancreatic beta cells |
| | | (from definitive endoderm stage) |
| Ppy | Pancreatic polypeptide | PP pancreatic cells |
| | | (gamma cells) |

Cardiac specific promoters

| | | |
|---|---|---|
| Myh6 | Myosin, heavy chain 6, cardiac | Late differentiation marker of cardiac |
| (aMHC) | muscle, alpha | muscle cells (atrial specificity) |
| MYL2 | Myosin, light chain 2, regulatory, | Late differentiation marker of cardiac |
| (MLC-2v) | cardiac, slow | muscle cells (ventricular specificity) |
| ITNNl3 | Troponin I type 3 (cardiac) | Cardiomyocytes |
| (cTnl) | | (from immature state) |
| ITNNl3 | Troponin I type 3 (cardiac) | Cardiomyocytes |
| (cTnl) | | (from immature state) |
| NPPA | Natriuretic peptide precursor A (also | Atrial specificity in adult cells |
| (ANF) | named Atrial Natriuretic Factor) | |
| Slc8a1 | Solute carrier family 8 | Cardiomyocytes from early |
| (Ncx1) | (sodium/calcium exchanger), member | developmental stages |
| | 1 | |

CNS specific promoters

| | | |
|---|---|---|
| SYN1 | Synapsin I | Neurons |
| (hSyn) | | |
| GFAP | Glial fibrillary acidic protein | Astrocytes |
| INA | Internexin neuronal intermediate | Neuroprogenitors |
| | filament protein, alpha (a-internexin) | |
| NES | Nestin | Neuroprogenitors and ectoderm |
| MOBP | Myelin-associated oligodendrocyte | Oligodendrocytes |
| | basic protein | |
| MBP | Myelin basic protein | Oligodendrocytes |
| TH | Tyrosine hydroxylase | Dopaminergic neurons |
| FOXA2 | Forkhead box A2 | Dopaminergic neurons (also used as a |
| (HNF3 | | marker of endoderm) |
| beta) | | |

Skin specific promoters

| | | |
|---|---|---|
| FLG | Filaggrin | Keratinocytes from granular layer |
| K14 | Keratin 14 | Keratinocytes from granular |
| | | and basal layers |
| TGM3 | Transglutaminase 3 | Keratinocytes from granular layer |

Immune cell specific promoters

| | | |
|---|---|---|
| ITGAM | Integrin, alpha M (complement | Monocytes, macrophages, granulocytes, |
| (CD11B) | component 3 receptor 3 subunit) | natural killer cells |

Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Fer114 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Oct4 | POU class 5 homeobox 1 | Pluripotent cells |
| (POU5F1) | | (germ cells, ES cells, iPS cells) |
| NANOG | Nanog homeobox | Pluripotent cells |
| | | (ES cells, iPS cells) |
| Synthetic | Synthetic promoter based on a Oct-4 | Pluripotent cells (ES cells, iPS cells) |
| Oct4 | core enhancer element | |
| T | Brachyury | Mesoderm |
| brachyury | | |
| NES | Nestin | Neuroprogenitors and Ectoderm |
| SOX17 | SRY (sex determining region Y)-box | Endoderm |
| | 17 | |
| FOXA2 | Forkhead box A2 | Endoderm (also used as a marker of |
| (HNFJ | | dopaminergic neurons) |
| beta) | | |
| MIR122 | MicroRNA 122 | Endoderm and hepatocytes |
| | | (from early stage embryonic liver cells~ |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544; incorporated herein by reference in its entirety).

In some embodiments, a nucleic acid encoding a Gene Writer or template nucleic acid is operably linked to a control element. e.g., a transcriptional control element, such as a promoter. The transcriptional control element may, in some embodiment, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter, a neurofilament promoter (see, e.g., GenBank HUMNFL, L04147); a synapsin promoter (see, e.g., GenBank H UMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TIH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnR H promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter, a Ca2+-calmodulin-dependent protein kinase II-alpha (CamKIIa) promoter (see, e.g., Mayford et al. (1996) Proc. Nati. Acad. Sci. USA 93:13250; and Casanova et al. (2001) Genesis 31:37); a CMV enhancer/platelet-derived growth factor-β promoter (see. e.g., Liu et al. (2004) Gene Therapy 11:52-60); and the like.

Adipocyte-specific spatially restricted promoters include, but are not limited to, the aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see. e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to, control sequences derived from the following genes: myosin light chain-2, α-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584-591: Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to, an SM22α promoter (see, e.g., Akyürek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM22α promoter, within which lie two CArG elements, has been shown to mediate vascular smooth muscle cell-specific expression (see. e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

Nonlimiting Exemplary Cell-Specific Promoters

Cell-specific promoters known in the art may be used to direct expression of a Gene Writer protein. e.g., as described herein. Nonlimiting exemplary mammalian cell-specific promoters have been characterized and used in mice expressing Cre recombinase in a cell-specific manner. Certain nonlimiting exemplary mammalian cell-specific promoters are listed in Table 1 of U.S. Pat. No. 9,845,481, incorporated herein by reference.

In some embodiments, the cell-specific promoter is a promoter that is active in plants. Many exemplary cell-specific plant promoters are known in the art. See, e.g., U.S. Pat. Nos. 5,097,025; 5,783,393; 5,880,330; 5,981,727; 7,557,264; 6,291,666; 7,132,526; and 7,323,622; and U.S. Publication Nos. 2010/0269226; 2007/0180580; 2005/0034192; and 2005/0086712, which are incorporated by reference herein in their entireties for any purpose.

In some embodiments, a vector as described herein comprises an expression cassette. The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In certain embodiments, the promoter is a heterologous promoter. The term “heterologous promoter”, as used herein, refers to a promoter that is not found to be operatively linked to a given encoding sequence in nature. In certain embodiments, an expression cassette may comprise additional elements, for example, an intron, an enhancer, a polyadenylation site, a woodchuck response element (WRE), and/or other elements known to affect expression levels of the encoding sequence. A “promoter” typically controls the expression of a coding sequence or functional RNA. In certain embodiments, a promoter sequence comprises proximal and more distal upstream elements and can further comprise an enhancer element. An “enhancer” can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoter include, but are not limited to, the phosphoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter: adenovirus major late promoter (Ad MLP); a herpes simplex virus (HSV) promoter, a cytomegalovirus (CMV) promoter such as the CMV immediate early promoter region (CMVIE). SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, Calif.). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof is used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities. In some cases, the tissue-specific regulatory sequences bind tissue-specific transcription factors that induce transcription in a tissue specific manner. Various tissue-specific regulatory sequences (e.g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter. Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter: T cell receptor α-chain promoter, neuronal such as neuron-specific enolase (NSI) promoter (Andersen et al., Cell. Mol. Neurobiol., 13:503-15 (1993)), neurofilament light-chain gene promoter (Piccioli et al., Proc. Nati. Acad. Sci. USA. 88:5611-5 (1991)), and the neuron-specific vgf gene promoter (Piccioli et al., Neuron. 15:373-84 (1995)), and others. Additional exemplary promoter sequences are described, for example, in U.S. patent Ser. No. 10/300,146 (incorporated herein by reference in its entirety). In some embodiments, a tissue-specific regulatory element, e.g., a tissue-specific promoter, is selected from one known to be operably linked to a gene that is highly expressed in a given tissue, e.g., as measured by RNA-seq or protein expression data, or a combination thereof. Methods for analyzing tissue specificity by expression are taught in Fagerberg et al. Mol Cell Proteomics 13(2):397-406 (2014), which is incorporated herein by reference in its entirety.

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a gene writer and gene writer template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

In some embodiments, the sequence encodes an RNA with a hairpin. In some embodiments, the hairpin RNA is a guide RNA, a template RNA, shRNA, or a microRNA. In some embodiments, the first promoter is an RNA polymerase I promoter. In some embodiments, the first promoter is an RNA polymerase II promoter. In some embodiments, the second promoter is an RNA polymerase III promoter. In some embodiments, the second promoter is a U6 or H1 promoter. In some embodiments, the nucleic acid construct comprises the structure of AAV construct B1 or B2.

Without wishing to be bound by theory, multicistronic expression constructs may not achieve optimal expression levels as compared to expression systems containing only one cistron. One of the suggested causes of lower expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S. Kaftansis L. Vassaux G. Direct comparison of the insulating properties of no genetic elements in an adenoviral vector containing two different expression cassettes. Hum Gene Ther. 2004 October; 1510):995-1002: both references incorporated herein by reference for disclosure of promoter interference phenomenon). In some embodiments, the problem of promoter interference may be overcome, e.g., by producing multicistronic expression constructs comprising only one promoter driving transcription of multiple encoding nucleic acid sequences separated by internal ribosomal entry sites, or by separating cistrons comprising their own promoter with transcriptional insulator elements. In some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

### MicroRNAs

miRNAs and other small interfering nucleic acids generally regulate gene expression via target RNA transcript cleavage/degradation or translational repression of the target messenger RNA (mRNA), miRNAs may, in some instances, be natively expressed, typically as final 19-25 non-translated RNA products, miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Ser. No. 10/300,146, 22:25-25:48, incorporated by reference. In some embodiments, one or more binding sites for one or more of the foregoing mi RNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety).

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods. Epub Aug. 12, 2007; incorporated by reference herein in its entirety). In some embodiments, microRNA sponges, or other miR inhibitors, are used with the AAVs, microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

### 5′ UTR and 3′ UTR

In certain embodiments, a nucleic acid comprising an open reading frame encoding a Gene Writer polypeptide (e.g., as described herein) comprises a 5′ UTR and/or a 3′ UTR. In embodiments, a 5′ UTR and 3′ UTR for protein expression, e.g., mRNA (or DNA encoding the RNA) for a Gene Writer polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC (SEQ ID NO: 1867) and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 1868), e.g., as described in Richner et al. Cell 168(6): P1114-1125 (2017), the sequences of which are incorporated herein by reference.

In some embodiments, an open reading frame of a Gene Writer system, e.g., an ORF of an mRNA (or DNA encoding an mRNA) encoding a Gene Writer polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′ (SEQ ID NO: 1869). In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′ (SEQ ID NO: 1870). This combination of 5′ UTR and 3′ UTR has been shown to result in desirable expression of an operably linked ORF by Richner et al. Cell 168(6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g, a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### Viral Vectors and Components Thereof

Viruses are a useful source of delivery vehicles for the systems described herein, in addition to a source of relevant enzymes or domains as described herein, e.g., as sources of recombinases and DNA binding domains used herein, e.g., Cre recombinase, lambda integrase, or the DNA binding domains from AAV Rep proteins. Some enzymes may have multiple activities. In some embodiments, the virus used as a Gene Writer delivery system or a source of components thereof may be selected from a group as described by Baltimore Bacteriol Rev 35(3):235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host.

In some embodiments, virions used to deliver nucleic acid in this invention may also carry enzymes involved in the process of Gene Writing. For example, a virion may contain a recombinase domain that is delivered into a host cell along with the nucleic acid. In some embodiments, a template nucleic acid may be associated with a Gene Writer polypeptide within a virion, such that both are co-delivered to a target cell upon transduction of the nucleic acid from the viral particle. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome. In some embodiments, a viral genome may comprise two or more nucleic acid molecules, e.g., comprise a segmented genome. In some embodiments, a nucleic acid in a virion may be associated with one or proteins. In some embodiments, one or more proteins in a virion may be delivered to a host cell upon transduction. In some embodiments, a natural virus may be adapted for nucleic acid delivery by the addition of virion packaging signals to the target nucleic acid, wherein a host cell is used to package the target nucleic acid containing the packaging signals.

In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

RNAs (e.g., a gRNA or an mRNA, e.g., an mRNA encoding a GeneWriter) may also be produced as described herein. In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gRNA is produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered, e.g., be less than 37° C. (e.g., between 0-10 C, 10-20 C, or 20-30 C), to result in a higher proportion of full-length transcripts (see Krieg Nucleic Acids Res 18:6463 (1990), which is herein incorporated by reference in its entirety). In some embodiments, a protocol for improved synthesis of long transcripts is employed to synthesize a long RNA, e.g., an RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a Gene Writer polypeptide) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC (SEQ ID NO: 1871) and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 1872), or functional fragments or variants thereof, and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

In some embodiments, RNA segments may be connected using an Azide-Alkyne Huisgen Cycloaddition, reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitation, the use of halogens (F—, Br—, I—)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

By way of illustration, a tracrRNA is typically around 80 nucleotides in length. Such RNA molecules may be produced, for example, by processes such as in vitro transcription or chemical synthesis. In some embodiments, when chemical synthesis is used to produce such RNA molecules, they may be produced as a single synthesis product or by linking two or more synthesized RNA segments to each other. In embodiments, when three or more RNA segments are connected to each other, different methods may be used to link the individual segments together. Also, the RNA segments may be connected to each other in one pot (e.g., a container, vessel, well, tube, plate, or other receptacle), all at the same time, or in one pot at different times or in different pots at different times. In a non-limiting example, to assemble RNA Segments 1, 2 and 3 in numerical order, RNA Segments 1 and 2 may first be connected, 5′ to 3′, to each other. The reaction product may then be purified for reaction mixture components (e.g., by chromatography), then placed in a second pot, for connection of the 3′ terminus with the 5′ terminus of RNA Segment 3. The final reaction product may then be connected to the 5′ terminus of RNA Segment 3.

In another non-limiting example, RNA Segment 1 (about 30 nucleotides) is the target locus recognition sequence of a crRNA and a portion of Hairpin Region 1. RNA Segment 2 (about 35 nucleotides) contains the remainder of Hairpin Region 1 and some of the linear tracrRNA between Hairpin Region 1 and Hairpin Region 2. RNA Segment 3 (about 35 nucleotides) contains the remainder of the linear tracrRNA between Hairpin Region 1 and Hairpin Region 2 and all of Hairpin Region 2. In this example, RNA Segments 2 and 3 are linked, 5′ to 3′, using click chemistry. Further, the 5′ and 3′ end termini of the reaction product are both phosphorylated. The reaction product is then contacted with RNA Segment 1, having a 3′ terminal hydroxyl group, and T4 RNA ligase to produce a guide RNA molecule.

A number of additional linking chemistries may be used to connect RNA segments according to method of the invention. Some of these chemistries are set out in Table 6 of US20160102322A1, which is incorporated herein by reference in its entirety.

### Vectors

The disclosure provides, in part, a nucleic acid, e.g., vector, encoding a Gene Writer polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a Gene Writer polypeptide is integrated into a target cell genome (e.g., upon administration to a target cell, tissue, organ, or subject). In some embodiments, a vector encoding a Gene Writer polypeptide is not integrated into a target cell genome (e.g., upon administration to a target cell, tissue, organ, or subject). In some embodiments, a vector comprising a template nucleic acid (e.g., template DNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a Gene Writer polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

AAV Vectors

In some embodiments, the vector encoding a Gene Writer polypeptide described herein, a template nucleic acid described herein, or both, is an adeno-associated virus (AAV) vector, e.g., comprising an AAV genome. In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a protein described herein and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a Gene Writer described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific Gene Writing, the expression of the Gene Writer and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a Gene Writer, promoter, and transcription terminator can fit into a single viral vector. SpCas9 (4.1 kb) may, in some instances, be difficult to package into AAV. Therefore, in some embodiments, a Gene Writer is used that is shorter in length than other Gene Writers or base editors. In some embodiments, the Gene Writers are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol. 82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrhlO, AAVLK03, AV10, AAV11, AAV 12, rhlO, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 7.

TABLE 7
Viral delivery modalities

| Target Tissue | Vehicle | Reference |
|---|---|---|
| Liver | AAV (AAV8¹, AAVrh.8¹, | 1. Wang et al., Mol. Ther. 18, |
| | AAVhu.37¹, AAV2/8, | 118-25 (2010) |
| | AAV2/rh10², AAV9, AAV2, | 2. Ginn et al., JHEP Reports, |
| | NP40³, NP59^{2,3}, AAV3B⁵, | 100065 (2019) |
| | AAV-DJ⁴, AAV-LK01⁴, | 3. Paulk et al., Mol. Ther. 26, |
| | AAV-LK02⁴, AAV-LK03⁴, | 289-303 (2018). |
| | AAV-LK19⁴ | 4. L. Lisowski et al., Nature. |
| | Adenovirus (Ad5, HC-AdV⁶) | 506, 382-6 (2014). |
| | | 5. L. Wang et al., Mol. Ther. |
| | | 23, 1877-87 (2015). |
| | | 6. Hausl Mol Ther (2010) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV6¹, AAV-LK19² | 1. Petek et al., Mol. Ther. |
| | | (2010) |
| | | 2. L. Lisowski et al., Nature. |
| | | 506, 382-6 (2014). |
| HSCs | HDAd5/35⁺⁺ | Wang et al. Blood Adv (2019) |

In some embodiments, a pharmaceutical composition (e.g., comprising an AAV as described herein) has less than 10% empty capsids, less than 8% empty capsids, less than 7% empty capsids, less than 5% empty capsids, less than 3% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ pg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ pg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10 5 pg by 1.0×10¹³ vg, less than 8.0×10⁵ pg by 1.0×10¹³ vg or less than 6.8×10 5 pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP <85> (incorporated by reference in its entirety) is less than 1.0 EU/mL, less than 0.8 EU/mL or less than 0.75 EU/mL. In embodiments, the osmolarity of a pharmaceutical composition according to USP, for example, USP <785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 m per container, less than 1000 particles that are greater than 25 m per container, less than 500 particles that are greater than 25 m per container or any intermediate value. In embodiments, the pharmaceutical composition contains less than 10,000 particles that are greater than 10 m per container, less than 8000 particles that are greater than 10 m per container or less than 600 particles that are greater than 10 pm per container.

In one embodiment, the pharmaceutical composition has a genomic titer of 0.5 to 5.0×10¹³ vg/mL, 1.0 to 4.0×10³ vg/mL, 1.5 to 3.0×10¹ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10⁵ pg of residual DNA plasmid per 1.0×10¹³ vg, less than about 1.1×10⁵ pg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 μm in size per container, less than about 6000 particles that are >10 m in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 pg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between ±15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### Kits, Articles of Manufacture, and Pharmaceutical Compositions

In an aspect the disclosure provides a kit comprising a Gene Writer or a Gene Writing system, e.g., as described herein. In some embodiments, the kit comprises a Gene Writer polypeptide (or a nucleic acid encoding the polypeptide) and a template DNA. In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), Gene Writers, and/or Gene Writer systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a Gene Writer or a Gene Writing system, e.g., as described herein. In some embodiments, the pharmaceutical composition further comprises a pharmaceutically acceptable carrier or excipient. In some embodiments, the pharmaceutical composition comprises a template DNA.

### Chemistry, Manufacturing, and Controls (CMC)

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a Gene Writer™ system, polypeptide, and/or template nucleic acid (e.g., template DNA) conforms to certain quality standards. In some embodiments, a Gene Writer™ system, polypeptide, and/or template nucleic acid (e.g., template DNA) produced by a method described herein conforms to certain quality standards. Accordingly, the disclosure is directed, in some aspects, to methods of manufacturing a Gene Writer™ system, polypeptide, and/or template nucleic acid that conforms to certain quality standards, e.g., in which said quality standards are assayed. The disclosure is also directed, in some aspects, to methods of assaying said quality standards in a Gene Writer™ system, polypeptide, and/or template nucleic acid. In some embodiments, quality standards include, but are not limited to, one or more (e.g., 1, 2, 3, 4, 5, 6, 7, 8, 9, 10, 11, or 12) of the following:

(i) the length of the template DNA or the mRNA encoding the GeneWriter polypeptide, e.g., whether the DNA or mRNA has a length that is above a reference length or within a reference length range, e.g., whether at least 80, 85, 90, 95, 96, 97, 98, or 99% of the DNA or mRNA present is greater than 100, 125, 150, 175, or 200 nucleotides long;

(ii) the presence, absence, and/or length of a polyA tail on the mRNA, e.g., whether at least 80, 85, 90, 95, 96, 97, 98, or 99% of the mRNA present contains a polyA tail (e.g., a polyA tail that is at least 5, 10, 20, 30, 50, 70, 100 nucleotides in length);

(iii) the presence, absence, and/or type of a 5′ cap on the mRNA, e.g., whether at least 80, 85, 90, 95, 96, 97, 98, or 99% of the mRNA present contains a 5′ cap, e.g., whether that cap is a 7-methylguanosine cap, e.g., a O-Me-m7G cap;

(iv) the presence, absence, and/or type of one or more modified nucleotides (e.g., selected from pseudouridine, dihydrouridine, inosine, 7-methylguanosine, 1-N-methylpseudouridine (1-Me-P), 5-methoxyuridine (5-MO-U), 5-methylcytidine (5mC), or a locked nucleotide) in the mRNA, e.g., whether at least 80, 85, 90, 95, 96, 97, 98, or 99% of the mRNA present contains one or more modified nucleotides;

(v) the stability of the template DNA or the mRNA (e.g., over time and/or under a pre-selected condition), e.g., whether at least 80, 85, 90, 95, 96, 97, 98, or 99% of the DNA or mRNA remains intact (e.g., greater than 100, 125, 150, 175, or 200 nucleotides long) after a stability test;

(vi) the potency of the template DNA or the mRNA in a system for modifying DNA, e.g., whether at least 1% of target sites are modified after a system comprising the DNA or mRNA is assayed for potency;

(vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);

(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof;

(ix) the presence, absence, and/or type of one or more artificial, synthetic, or non-canonical amino acids (e.g., selected from ornithine, β-alanine, GABA, 6-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;

(x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;

(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or

(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:

(a) less than 1% (e.g., less than 0.5%, 0.4%, 0.3%, 0.2%, or 0.1%) DNA template relative to the RNA encoding the polypeptide, e.g., on a molar basis;

(b) less than 1% (e.g., less than 0.5%, 0.4%, 0.3%, 0.2%, or 0.1%) uncapped RNA relative to the RNA encoding the polypeptide, e.g., on a molar basis;

(c) less than 1% (e.g., less than 0.5%, 0.4%, 0.3%, 0.2%, or 0.1%) partial length RNAs relative to the RNA encoding the polypeptide, e.g., on a molar basis;

(d) substantially lacks unreacted cap dinucleotides.

### Exemplary Heterologous Object Sequences

In some embodiments, the systems or methods provided herein comprise a heterologous object sequence, wherein the heterologous object sequence or a reverse complementary sequence thereof, encodes a protein (e.g., an antibody) or peptide. In some embodiments, the therapy is one approved by a regulatory agency such as FDA.

In some embodiments, the protein or peptide is a protein or peptide from the THPdb database (Usmani et al. PLoS One 12(7):e0181748 (2017), herein incorporated by reference in its entirety. In some embodiments, the protein or peptide is a protein or peptide disclosed in Table 8. In some embodiments, the systems or methods disclosed herein, for example, those comprising Gene Writers, may be used to integrate an expression cassette for a protein or peptide from Table 8 into a host cell to enable the expression of the protein or peptide in the host. In some embodiments, the sequences of the protein or peptide in the first column of Table 8 can be found in the patents or applications provided in the third column of Table 8, incorporated by reference in their entireties.

In some embodiments, the protein or peptide is an antibody disclosed in Table 1 of Lu et al. J Biomed Sci 27(1):1 (2020), herein incorporated by reference in its entirety. In some embodiments, the protein or peptide is an antibody disclosed in Table 9. In some embodiments, the systems or methods disclosed herein, for example, those comprising Gene Writers, may be used to integrate an expression cassette for an antibody from Table 9 into a host cell to enable the expression of the antibody in the host. In some embodiments, a system or method described herein is used to express an agent that binds a target of column 2 of Table 9 (e.g., a monoclonal antibody of column 1 of Table 9) in a subject having an indication of column 3 of Table 9.

TABLE 8
Exemplary protein and peptide therapeutics.

| Therapeutic peptide | Category | Patent Number |
|---|---|---|
| Lepirudin | Antithrombins and Fibrinolytic | CA1339104 |
| | Agents | |
| Cetuximab | Antineoplastic Agents | CA1340417 |
| Dor se alpha | Enzymes | CA2184581 |
| Denileukin diftitox | Antineoplastic Agents | |
| Etanercept | Immunosuppressive Agents | CA2476934 |
| Bivalirudin | Antithrombins | US7582727 |
| Leuprolide | Antineoplastic Agents | |
| Peginterferon alpha-2a | Immunosuppressive Agents | CA2203480 |
| Alteplase | Thrombolytic Agents | |
| Interferon alpha-n1 | Antiviral Agents | |
| Darbepoetin alpha | Anti-anemic Agents | CA2165694 |
| Reteplase | Fibrinolytic Agents | CA2107476 |
| Epoetin alpha | Hematinics | CA1339047 |
| Salmon Calcitonin | Bone Density Conservation | US6440392 |
| | Agents | |
| Interferon alpha-n3 | Immunosuppressive Agents | |
| Pegfilgrastim | Immunosuppressive Agents | CA1341537 |
| Sargramostim | Immunosuppressive Agents | CA1341150 |
| Secretin | Diagnostic Agents | |
| Peginterferon alpha-2b | Immunosuppressive Agents | CA1341567 |
| Asparagi se | Antineoplastic Agents | |
| Thyrotropin alpha | Diagnostic Agents | US5840566 |
| Antihemophilic Factor | Coagulants and Thrombotic agents | CA2124690 |
| A kinra | Antirheumatic Agents | CA2141953 |
| Gramicidin D | Anti-Bacterial Agents | |
| Intravenous | Immunologic Factors | |
| Immunoglobulin | | |
| Anistreplase | Fibrinolytic Agents | |
| Insulin Regular | Antidiabetic Agents | |
| Tenecteplase | Fibrinolytic Agents | CA2129660 |
| Menotropins | Fertility Agents | |
| Interferon gamma-1b | Immunosuppressive Agents | US6936695 |
| Interferon alpha-2a, | | CA2172664 |
| Recombi nt | | |
| Coagulation factor VIIa | Coagulants | |
| Oprelvekin | Antineoplastic Agents | |
| Palifermin | Anti-Mucositis Agents | |
| Glucagon recombi nt | Hypoglycemic Agents | |
| Aldesleukin | Antineoplastic Agents | |
| Botulinum Toxin Type B | Antidystonic Agents | |
| Omalizumab | Anti-Allergic Agents | CA2113813 |
| Lutropin alpha | Fertility Agents | US5767251 |
| Insulin Lispro | Hypoglycemic Agents | US5474978 |
| Insulin Glargine | Hypoglycemic Agents | US7476652 |
| Collage se | | |
| Rasburicase | Gout Suppressants | CA2175971 |
| Adalimumab | Antirheumatic Agents | CA2243459 |
| Imiglucerase | Enzyme Replacement Agents | US5549892 |
| Abciximab | Anticoagulants | CA1341357 |
| Alpha-1-protei se inhibitor | Serine Protei se Inhibitors | |
| Pegaspargase | Antineoplastic Agents | |
| Interferon beta-1a | Antineoplastic Agents | CA1341604 |
| Pegademase bovine | Enzyme Replacement Agents | |
| Human Serum Albumin | Serum substitutes | US6723303 |
| Eptifibatide | Platelet Aggregation Inhibitors | US6706681 |
| Serum albumin iodo ted | Diagnostic Agents | |
| Infliximab | Antirheumatic Agents, Anti- | CA2106299 |
| | Inflammatory Agents, Non- | |
| | Steroidal, Dermatologic Agents, | |
| | Gastrointesti 1 Agents and | |
| | Immunosuppressive Agents | |
| Follitropin beta | Fertility Agents | US7741268 |
| Vasopressin | Antidiuretic Agents | |
| Interferon beta-1b | Adjuvants, Immunologic and | CA1340861 |
| | Immunosuppressive Agents | |
| Interferon alphacon-1 | Antiviral Agents and | CA1341567 |
| | Immunosuppressive Agents | |
| Hyaluronidase | Adjuvants, Anesthesia and | |
| | Permeabilizing Agents | |
| Insulin, porcine | Hypoglycemic Agents | |
| Trastuzumab | Antineoplastic Agents | CA2103059 |
| Rituximab | Antineoplastic Agents, | CA2149329 |
| | Immunologic Factors and | |
| | Antirheumatic Agents | |
| Basiliximab | Immunosuppressive Agents | CA2038279 |
| Muromo b | Immunologic Factors and | |
| | Immunosuppressive Agents | |
| Digoxin Immune Fab | Antidotes | |
| (Ovine) | | |
| Ibritumomab | | CA2149329 |
| Daptomycin | | US6468967 |
| Tositumomab | | |
| Pegvisomant | Hormone Replacement Agents | US5849535 |
| Botulinum Toxin Type A | Neuromuscular Blocking Agents, | CA2280565 |
| | Anti-Wrinkle Agents and | |
| | Antidystonic Agents | |
| Pancrelipase | Gastrointesti 1 Agents and Enzyme | |
| | Replacement Agents | |
| Streptoki se | Fibrinolytic Agents and | |
| | Thrombolytic Agents | |
| Alemtuzumab | | CA1339198 |
| Alglucerase | Enzyme Replacement Agents | |
| Capromab | Indicators, Reagents and | |
| | Diagnostic Agents | |
| Laronidase | Enzyme Replacement Agents | |
| Urofollitropin | Fertility Agents | US5767067 |
| Efalizumab | Immunosuppressive Agents | |
| Serum albumin | Serum substitutes | US6723303 |
| Choriogo dotropin alpha | Fertility Agents and Go dotropins | US6706681 |
| Antithymocyte globulin | Immunologic Factors and | |
| | Immunosuppressive Agents | |
| Filgrastim | Immunosuppressive Agents, | CA1341537 |
| | Antineutropenic Agents and | |
| | Hematopoietic Agents | |
| Coagulation factor ix | Coagulants and Thrombotic | |
| | Agents | |
| Becaplermin | Angiogenesis Inducing Agents | CA1340846 |
| Agalsidase beta | Enzyme Replacement Agents | CA2265464 |
| Interferon alpha-2b | Immunosuppressive Agents | CA1341567 |
| Oxytocin | Oxytocics, Anti-tocolytic Agents | |
| | and Labor Induction Agents | |
| Enfuvirtide | HIV Fusion Inhibitors | US6475491 |
| Palivizumab | Antiviral Agents | CA2197684 |
| Daclizumab | Immunosuppressive Agents | |
| Bevacizumab | Angiogenesis Inhibitors | CA2286330 |
| Arcitumomab | Diagnostic Agents | US8420081 |
| Arcitumomab | Diagnostic Agents | US7790142 |
| Eculizumab | | CA2189015 |
| Panitumumab | | |
| Ranibizumab | Ophthalmics | CA2286330 |
| Idursulfase | Enzyme Replacement Agents | |
| Alglucosidase alpha | Enzyme Replacement Agents | CA2416492 |
| Exe tide | Hypoglycemic Agents | US6872700 |
| Mecasermin | | US5681814 |
| Pramlintide | | US5686411 |
| Galsulfase | Enzyme Replacement Agents | |
| Abatacept | Antirheumatic Agents and | CA2110518 |
| | Immunosuppressive Agents | |
| Cosyntropin | Hormones and Diagnostic Agents | |
| Corticotropin | | |
| Insulin aspart | Hypoglycemic Agents and | US5866538 |
| | Antidiabetic Agents | |
| Insulin detemir | Antidiabetic Agents | US5750497 |
| Insulin glulisine | Antidiabetic Agents | US6960561 |
| Pegaptanib | Intended for the prevention of | |
| | respiratory distress syndrome | |
| | (RDS) in premature infants at high | |
| | risk for RDS. | |
| Nesiritide | | |
| Thymalphasin | | |
| Defibrotide | Antithrombins | |
| tural alpha interferon OR | | |
| multiferon | | |
| Glatiramer acetate | | |
| Preotact | | |
| Teicoplanin | Anti-Bacterial Agents | |
| Ca kinumab | Anti-Inflammatory Agents and | |
| | Monoclo 1 antibodies | |
| Ipilimumab | Antineoplastic Agents and | CA2381770 |
| | Monoclo 1 antibodies | |
| Sulodexide | Antithrombins and Fibrinolytic | |
| | Agents and Hypoglycemic Agents | |
| | and Anticoagulants and | |
| | Hypolipidemic Agents | |
| Tocilizumab | | CA2201781 |
| Teriparatide | Bone Density Conservation | US6977077 |
| | Agents | |
| Pertuzumab | Monoclo 1 antibodies | CA2376596 |
| Rilo cept | Immunosuppressive Agents | US5844099 |
| Denosumab | Bone Density Conservation | CA2257247 |
| | Agents and Monoclo 1 antibodies | |
| Liraglutide | | US6268343 |
| Golimumab | Antipsoriatic Agents and Monoclo | |
| | 1 antibodies and TNF inhibitor | |
| Belatacept | Antirheumatic Agents and | |
| | Immunosuppressive Agents | |
| Buserelin | | |
| Velaglucerase alpha | Enzymes | US7138262 |
| Tesamorelin | | US5861379 |
| Brentuximab vedotin | | |
| Taliglucerase alpha | Enzymes | |
| Belimumab | Monoclo 1 antibodies | |
| Aflibercept | Antineoplastic Agents and | US7306799 |
| | Ophthalmics | |
| Asparagi se erwinia | Enzymes | |
| chrysanthemi | | |
| Ocriplasmin | Ophthalmics | |
| Glucarpidase | Enzymes | |
| Teduglutide | | US5789379 |
| Raxibacumab | Anti-Infective Agents and | |
| | Monoclo 1 antibodies | |
| Certolizumab pegol | TNF inhibitor | CA2380298 |
| Insulin, isophane | Hypoglycemic Agents and | |
| | Antidiabetic Agents | |
| Epoetin zeta | | |
| Obinutuzumab | Antineoplastic Agents | |
| Fibrinolysin aka plasmin | | US3234106 |
| Follitropin alpha | | |
| Romiplostim | Colony-Stimulating Factors and | |
| | Thrombopoietic Agents | |
| Luci ctant | Pulmo ry surfactants | US5407914 |
| talizumab | Immunosuppressive agents | |
| Aliskiren | Renin inhibitor | |
| Ragweed Pollen Extract | | |
| Secukinumab | Inhibitor | US20130202610 |
| Somatotropin Recombi nt | Hormone Replacement Agents | CA1326439 |
| Drotrecogin alpha | Antisepsis | CA2036894 |
| Alefacept | Dermatologic and | |
| | Immunosupressive agents | |
| OspA lipoprotein | Vaccines | |
| Uroki se | | US4258030 |
| Abarelix | Anti-Testosterone Agents | US5968895 |
| Sermorelin | Hormone Replacement Agents | |
| Aprotinin | | US5198534 |
| Gemtuzumab ozogamicin | Antineoplastic agents and | US5585089 |
| | Immunotoxins | |
| Satumomab Pendetide | Diagnostic Agents | |
| Albiglutide | Drugs used in diabetes; alimentary | |
| | tract and metabolism; blood | |
| | glucose lowering drugs, excl. | |
| | insulins. | |
| Alirocumab | | |
| Ancestim | | |
| Antithrombin alpha | | |
| Antithrombin III human | | |
| Asfotase alpha | Enzymes Alimentary Tract and | |
| | Metabolism | |
| Atezolizumab | | |
| Autologous cultured | | |
| chondrocytes | | |
| Beractant | | |
| Bli tumomab | Antineoplastic Agents | US20120328618 |
| | Immunosuppressive Agents | |
| | Monoclo 1 antibodies | |
| | Antineoplastic and | |
| | Immunomodulating Agents | |
| C1 Esterase Inhibitor | | |
| (Human) | | |
| Coagulation Factor XIII A- | | |
| Subunit (Recombi nt) | | |
| Conestat alpha | | |
| Daratumumab | Antineoplastic Agents | |
| Desirudin | | |
| Dulaglutide | Hypoglycemic Agents; Drugs | |
| | Used in Diabetes; Alimentary | |
| | Tract and Metabolism; Blood | |
| | Glucose Lowering Drugs, Excl. | |
| | Insulins | |
| Elosulfase alpha | Enzymes; Alimentary Tract and | |
| | Metabolism | |
| Elotuzumab | | US2014055370 |
| Evolocumab | Lipid Modifying Agents, Plain; | |
| | Cardiovascular System | |
| Fibrinogen Concentrate | | |
| (Human) | | |
| Filgrastim-sndz | | |
| Gastric intrinsic factor | | |
| Hepatitis B immune | | |
| globulin | | |
| Human calcitonin | | |
| Human Clostridium tetani | | |
| toxoid immune globulin | | |
| Human rabies virus | | |
| immune globulin | | |
| Human Rho(D) immune | | |
| globulin | | |
| Hyaluronidase (Human | | US7767429 |
| Recombi nt) | | |
| Idarucizumab | Anticoagulant | |
| Immune Globulin Human | Immunologic Factors; | |
| | Immunosuppressive Agents; Anti- | |
| | Infective Agents | |
| Vedolizumab | Immunosupressive agent, | US2012151248 |
| | Antineoplastic agent | |
| Ustekinumab | Deramtologic agent, | |
| | Immunosuppressive agent, | |
| | antineoplastic agent | |
| Turoctocog alpha | | |
| Tuberculin Purified Protein | | |
| Derivative | | |
| Simoctocog alpha | Antihaemorrhagics: blood | |
| | coagulation factor VIII | |
| Siltuximab | Antineoplastic and | US7612182 |
| | Immunomodulating Agents, | |
| | Immunosuppressive Agents | |
| Sebelipase alpha | Enzymes | |
| Sacrosidase | Enzymes | |
| Ramucirumab | Antineoplastic and | US2013067098 |
| | Immunomodulating Agents | |
| Prothrombin complex | | |
| concentrate | | |
| Poractant alpha | Pulmo ry Surfactants | |
| Pembrolizumab | Antineoplastic and | US2012135408 |
| | Immunomodulating Agents | |
| Peginterferon beta-1a | | |
| Ofatumumab | Antineoplastic and | US8337847 |
| | Immunomodulating Agents | |
| Obiltoxaximab | | |
| Nivolumab | Antineoplastic and | US2013173223 |
| | Immunomodulating Agents | |
| Necitumumab | | |
| Metreleptin | | US20070099836 |
| Methoxy polyethylene | | |
| glycol-epoetin beta | | |
| Mepolizumab | Antineoplastic and | US2008134721 |
| | Immunomodulating Agents, | |
| | Immunosuppressive Agents, | |
| | Interleukin Inhibitors | |
| Ixekizumab | | |
| Insulin Pork | Hypoglycemic Agents, | |
| | Antidiabetic Agents | |
| Insulin Degludec | | |
| Insulin Beef | | |
| Thyroglobulin | Hormone therapy | US5099001 |
| Anthrax immune globulin | Plasma derivative | |
| human | | |
| Anti-inhibitor coagulant | Blood Coagulation Factors, | |
| complex | Antihemophilic Agent | |
| Anti-thymocyte Globulin | Antibody | |
| (Equine) | | |
| Anti-thymocyte Globulin | Antibody | |
| (Rabbit) | | |
| Brodalumab | Antineoplastic and | |
| | Immunomodulating Agents | |
| C1 Esterase Inhibitor | Blood and Blood Forming Organs | |
| (Recombi nt) | | |
| Ca kinumab | Antineoplastic and | |
| | Immunomodulating Agents | |
| Chorionic Go dotropin | Hormones | US6706681 |
| (Human) | | |
| Chorionic Go dotropin | Hormones | US5767251 |
| (Recombi nt) | | |
| Coagulation factor X | Blood Coagulation Factors | |
| human | | |
| Dinutuximab | Antibody, Immunosuppresive | US20140170155 |
| | agent, Antineoplastic agent | |
| Efmoroctocog alpha | Antihemophilic Factor | |
| Factor IX Complex | Antihemophilic agent | |
| (Human) | | |
| Hepatitis A Vaccine | Vaccine | |
| Human Varicella-Zoster | Antibody | |
| Immune Globulin | | |
| Ibritumomab tiuxetan | Antibody, Immunosuppressive | CA2149329 |
| | Agents | |
| Lenograstim | Antineoplastic and | |
| | Immunomodulating Agents | |
| Pegloticase | Enzymes | |
| Protamine sulfate | Heparin Antagonists, Hematologic | |
| | Agents | |
| Protein S human | Anticoagulant plasma protein | |
| Sipuleucel-T | Antineoplastic and | US8153120 |
| | Immunomodulating Agents | |
| Somatropin recombi nt | Hormones, Hormone Substitutes, | CA1326439, CA2252535, |
| | and Hormone Antagonists | US5288703, US5849700, |
| | | US5849704, US5898030, |
| | | US6004297, US6152897, |
| | | US6235004, US6899699 |
| Susoctocog alpha | Blood coagulation factors, | |
| | Antihaemorrhagics | |
| Thrombomodulin alpha | Anticoagulant agent, Antiplatelet | |
| | agent | |

TABLE 9
Exemplary monoclonal antibody therapies.

| mAb | Target | Indication |
|---|---|---|
| Muromonab-CD3 | CD3 | Kidney transplant rejection |
| Abeiximab | GPIIb/IIIa | Prevention of blood clots in angioplasty |
| Rituximab | CD20 | Non-Hodgkin lymphoma |
| Palivizumab | RSV | Prevention of respiratory syncytial virus |
| | | infection |
| Infliximab | TNFα | Crohn's disease |
| Trastuzumab | HER2 | Breast cancer |
| Alemtuzumab | CD52 | Chronic myeloid leukemia |
| Adalimumab | TNFα | Rheumatoid arthritis |
| Ibritumomab | CD20 | Non-Hodgkin lymphoma |
| tiuxetan | | |
| Omalizumab | IgE | Asthma |
| Cetuximab | EGFR | Colorectal cancer |
| Bevacizumab | VEGF-A | Colorectal cancer |
| Natalizumab | ITGA4 | Multiple sclerosis |
| Panitumumab | EGFR | Colorectal cancer |
| Ranibizumab | VEGF-A | Macular degeneration |
| Eculizumab | C5 | Paroxysmal nocturnal hemoglobinuria |
| Certolizumab | TNFα | Crohn's disease |
| pegol | | |
| Ustekinumab | IL-12/23 | Psoriasis |
| Canakinumab | IL-1β | Muckle-Wells syndrome |
| Golimumab | TNFα | Rheumatoid and psoriatic arthritis, ankylosing |
| | | spondylitis |
| Ofatumumab | CD20 | Chronic lymphocytic leukemia |
| Tocilizumab | IL-6R | Rheumatoid arthritis |
| Denosumab | RANKL | Bone loss |
| Belimumab | BLyS | Systemic lupus erythematosus |
| Ipilimumab | CTLA-4 | Metastatic melanoma |
| Brentuximab | CD30 | Hodgkin lymphoma, systemic anaplastic large |
| vedotin | | cell lymphoma |
| Pertuzumab | HER2 | Breast Cancer |
| Trastuzumab | HER2 | Breast cancer |
| emtansine | | |
| Raxibacumab | B. anthrasis PA | Anthrax infection |
| Obinutuzumab | CD20 | Chronic lymphocytic leukemia |
| Siltuximab | IL-6 | Castleman disease |
| Ramucirumab | VEGFR2 | Gastric cancer |
| Vedolizumab | α4β7 integrin | Ulcerative colitis, Crohn disease |
| Blinatumomab | CD19, CD3 | Acute lymphoblastic leukemia |
| Nivolumab | PD-1 | Melanoma, non-small cell lung cancer |
| Pembrolizumab | PD-1 | Melanoma |
| Idarucizumab | Dabigatran | Reversal of dabigatran-induced anticoagulation |
| Necitumumab | EGFR | Non-small cell lung cancer |
| Dinutuximab | GD2 | Neuroblastoma |
| Secukinumab | IL-17α | Psoriasis |
| Mepolizumab | IL-5 | Severe eosinophilic asthma |
| Alirocumab | PCSK9 | High cholesterol |
| Evolocumab | PCSK9 | High cholesterol |
| Daratumumab | CD38 | Multiple myeloma |
| Elotuzumab | SLAMF7 | Multiple myeloma |
| Ixekizumab | IL-17α | Psoriasis |
| Reslizumab | IL-5 | Asthma |
| Olaratumab | PDGFRα | Soft tissue sarcoma |
| Bezlotoxumab | Clostridium | Prevention of Clostridium difficile infection |
| | difficile enterotoxin B | recurrence |
| Atezolizumab | PD-L1 | Bladder cancer |
| Obiltoxaximab | B. anthrasis PA | Prevention of inhalational anthrax |
| Inotuzumab | CD22 | Acute lymphoblastic leukemia |
| ozogamicin | | |
| Brodalumab | IL-17R | Plaque psoriasis |
| Guselkumab | IL-23 p19 | Plaque psoriasis |
| Dupilumab | IL-4Rα | Atopic dermatitis |
| Sarilumab | IL-6R | Rheumatoid arthritis |
| Avelumab | PD-L1 | Merkel cell carcinoma |
| Ocrelizumab | CD20 | Multiple sclerosis |
| Emicizumab | Factor IXa, X | Hemophilia A |
| Benralizumab | IL-5Rα | Asthma |
| Gemtuzumab | CD33 | Acute myeloid leukemia |
| ozogamicin | | |
| Durvalumab | PD-L1 | Bladder cancer |
| Burosumab | FGF23 | X-linked hypophosphatemia |
| Lanadelumab | Plasma kallikrein | Hereditary angioedema attacks |
| Mogamulizumab | CCR4 | Mycosis fungoides or Sézary syndrome |
| Erenumab | CGRPR | Migraine prevention |
| Galcanezumab | CGRP | Migraine prevention |
| Tildrakizumab | IL-23 p19 | Plaque psoriasis |
| Cemiplimab | PD-1 | Cutaneous squamous cell carcinoma |
| Emapalumab | IFNγ | Primary hemophagocytic lymphohistiocytosis |
| Fremanezumab | CGRP | Migraine prevention |
| Ibalizumab | CD4 | HIV infection |
| Moxetumomab | CD22 | Hairy cell leukemia |
| pasudodox | | |
| Ravulizumab | C5 | Paroxysmal nocturnal hemoglobinuria |
| Caplacizumab | von Willebrand factor | Acquired thrombotic thrombocytopenic purpura |
| Romosozumab | Selerostin | Osteoporosis in postmenopausal women at |
| | | increased risk of fracture |
| Risankizumab | IL-23 p19 | Plaque psoriasis |
| Polatuzumab | CD79β | Diffuse large B-cell lymphoma |
| vedotin | | |
| Brolucizumab | VEGF-A | Macular degeneration |
| Crizanlizumab | P-selectin | Sickle cell disease |

### Applications

By integrating coding genes into a DNA sequence template, the Gene Writer system can address therapeutic needs, for example, by providing expression of a therapeutic transgene (e.g., comprised in an object sequence as described herein) in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, an object sequence (e.g., a heterologous object sequence) comprises a coding sequence encoding a functional element (e.g., a polypeptide or non-coding RNA, e.g., as described herein) specific to the therapeutic needs of the host cell. In some embodiments, an object sequence (e.g., a heterologous object sequence) comprises a promoter, for example, a tissue specific promotor or enhancer. In some embodiments, a promotor can be operably linked to a coding sequence.

In embodiments, the Gene Writer™ gene editor system can provide an object sequence comprising, e.g., a therapeutic agent (e.g., a therapeutic transgene) expressing, e.g., replacement blood factors or replacement enzymes, e.g., lysosomal enzymes. For example, the compositions, systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease. For example, the compositions, systems and methods described herein are useful to express, in a target human genome factor I, II, V, VII, X, XI, XII or XIII for blood factor deficiencies.

### Administration

The composition and systems described herein may be used in vitro or in vivo. In some embodiments the system or components of the system are delivered to cells (e.g., mammalian cells, e.g., human cells), e.g., in vitro or in vivo. The skilled artisan will understand that the components of the Gene Writer system may be delivered in the form of polypeptide, nucleic acid (e.g., DNA, RNA), and combinations thereof.

In some embodiments, the system and/or components of the system are delivered as nucleic acids. For example, the recombinase polypeptide may be delivered in the form of a DNA or RNA encoding the recombinase polypeptide. In some embodiments the system or components of the system (e.g., an insert DNA and a recombinase polypeptide-encoding nucleic acid molecule) are delivered on 1, 2, 3, 4, or more distinct nucleic acid molecules. In some embodiments the system or components of the system are delivered as a combination of DNA and RNA. In some embodiments the system or components of the system are delivered as a combination of DNA and protein. In some embodiments the system or components of the system are delivered as a combination of RNA and protein. In some embodiments the recombinase polypeptide is delivered as a protein.

In some embodiments the system or components of the system are delivered to cells, e.g. mammalian cells or human cells, using a vector. The vector may be, e.g., a plasmid or a virus. In some embodiments delivery is in vivo, in vitro, ex vivo, or in situ. In some embodiments the virus is an adeno associated virus (AAV), a lentivirus, an adenovirus. In some embodiments the system or components of the system are delivered to cells with a viral-like particle or a virosome. In some embodiments the delivery uses more than one virus, viral-like particle or virosome.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

In some embodiments, at least one component of a system described herein comprises a fusosome. Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see, for example, the sections relating to fusosome design, preparation, and usage in PCT Publication No. WO/2020014209, incorporated herein by reference in its entirety).

A Gene Writer system can be introduced into cells, tissues and multicellular organisms. In some embodiments the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some embodiments, a Gene Writer™ system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a Gene Writer™ system described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a Gene Writer™ system described herein described herein is administered by enteral administration (e.g. oral, rectal, gastrointestinal, sublingual, sublabial, or buccal administration). In some embodiments, a Gene Writer™ system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a Gene Writer™ system described herein is administered by topical administration (e.g., transdermal administration).

In some embodiments, a Gene Writer™ system as described herein can be used to modify an animal cell, plant cell, or fungal cell. In some embodiments, a Gene Writer™ system as described herein can be used to modify a mammalian cell (e.g., a human cell). In some embodiments, a Gene Writer™ system as described herein can be used to modify a cell from a livestock animal (e.g., a cow, horse, sheep, goat, pig, llama, alpaca, camel, yak, chicken, duck, goose, or ostrich). In some embodiments, a Gene Writer™ system as described herein can be used as a laboratory tool or a research tool, or used in a laboratory method or research method, e.g., to modify an animal cell, e.g., a mammalian cell (e.g., a human cell), a plant cell, or a fungal cell.

In some embodiments, a Gene Writer™ system as described herein can be used to express a protein, template, or heterologous object sequence (e.g., in an animal cell, e.g., a mammalian cell (e.g., a human cell), a plant cell, or a fungal cell). In some embodiments, a Gene Writer™ system as described herein can be used to express a protein, template, or heterologous object sequence under the control of an inducible promoter (e.g., a small molecule inducible promoter). In some embodiments, a Gene Writing system or payload thereof is designed for tunable control, e.g., by the use of an inducible promoter. For example, a promoter, e.g., Tet, driving a gene of interest may be silent at integration, but may, in some instances, activated upon exposure to a small molecule inducer, e.g., doxycycline. In some embodiments, the tunable expression allows post-treatment control of a gene (e.g., a therapeutic gene), e.g., permitting a small molecule-dependent dosing effect. In embodiments, the small molecule-dependent dosing effect comprises altering levels of the gene product temporally and/or spatially, e.g., by local administration. In some embodiments, a promoter used in a system described herein may be inducible, e.g., responsive to an endogenous molecule of the host and/or an exogenous small molecule administered thereto.

### Treatment of Suitable Indications

In some embodiments, a Gene Writer™ system described herein, or a component or portion thereof (e.g., a polypeptide or nucleic acid as described herein), is used to treat a disease, disorder, or condition. In some embodiments, the Gene Writer™ system described herein, or component or portion thereof, is used to treat a disease, disorder, or condition listed in any of Tables 10-15. In some embodiments, the Gene Writer™ system described herein, or component or portion thereof, is used to treat a hematopoietic stem cell (HSC) disease, disorder, or condition, e.g., as listed in Table 10. In some embodiments, the Gene Writer™ system described herein, or component or portion thereof, is used to treat a kidney disease, disorder, or condition, e.g., as listed in Table 11. In some embodiments, the Gene Writer™ system described herein, or component or portion thereof, is used to treat a liver disease, disorder, or condition, e.g., as listed in Table 12. In some embodiments, the Gene Writer™ system described herein, or component or portion thereof, is used to treat a lung disease, disorder, or condition, e.g., as listed in Table 13. In some embodiments, the Gene Writer™ system described herein, or component or portion thereof, is used to treat a skeletal muscle disease, disorder, or condition, e.g., as listed in Table 14. In some embodiments, the Gene Writer™ system described herein, or component or portion thereof, is used to treat a skin disease, disorder, or condition, e.g., as listed in Table 15.

Tables 10-15: Indications Selected for Trans Gene Writers to be Used for Recombinases

TABLE 10
HSCs

| Disease | Gene Affected |
|---|---|
| Adrenoleukodystrophy (CALD) | ABCD1 |
| Alpha-mannosidosis | MAN2B1 |
| Fanconi anemia | FANCA; FANCC; FANCG |
| Gaucher disease | GBA |
| Globoid cell leukodystrophy (Krabbe disease) | GALC |
| Hemophagocytic lymphohistiocytosis | PRF1; STX11; STXBP2; UNC13D |
| Malignant infantile osteopetrosis-autosomal | TCIRG1; Many genes implicated |
| recessive osteopetrosis | |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Pompe disease | GAA |
| Sickle cell disease (SCD) | HBB |
| Tay Sachs | HEXA |
| Thalassemia | HBB |

TABLE 11
Kidney

| Disease | Gene Affected |
|---|---|
| Congenital nephrotic syndrome | NPHS2 |
| Cystinosis | CTNS |

TABLE 12
Liver

| Disease | Gene Affected |
|---|---|
| Acute intermittent porphyria | HMBS |
| Alagille syndrome | JAG1 |
| Carbamoyl phosphate synthetase I deficiency | CPS1 |
| Citrullinemia I | ASS1 |
| Crigler-Najjar | UGT1A1 |
| Fabry | LPL |
| Familial chylomicronemia syndrome | GLA |
| Gaucher | GBE1 |
| GSD IV | GBA |
| Heme A | F8 |
| Heme B | F9 |
| HoFH | LDLRAP1 |
| Methylmalonic acidemia | Type Ia: BCKDHA |
| | Type Ib: BCKDHB |
| | Type II: DBT |
| MPS II | MMUT |
| MPS III | IDS |
| MPS IV | Type IIIa: SGSH |
| | Type IIIb: NAGLU |
| | Type IIIc: HGSNAT |
| | Type IIId: GNS |
| MPS VI | Type IVA: GALNS |
| | Type IVB: GLB1 |
| MSUD | ARSB |
| OTC Deficiency | OTC |
| Polycystic Liver Disease | PRKCSH |
| Pompe | GAA |
| Primary Hyperoxaluria 1 | AGXT (HAO1 or LDHA for CRISPR) |
| Progressive familial intrahepatic cholestasis type 1 | ATP8B1 |
| Progressive familial intrahepatic cholestasis type 2 | ABCB11 |
| Progressive familial intrahepatic cholestasis type 3 | ABCB4 |
| Propionic acidemia | PCCB; PCCA |
| Wilson's Disease | ATP7B |

TABLE 13
Lung

| Disease | Gene Affected |
|---|---|
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency | SFTPB |
| (pulmonary surfactant metabolism dysfunction 1) | |

TABLE 14
Skeletal muscle

| Disease | Gene Affected |
|---|---|
| Becker muscular dystrophy | DMD |
| Becker myotonia | CLCN1 |
| Bethlem myopathy | COL6A2 |
| Centronuclear myopathy, X-linked (motubular) | MTM1 |
| Congenital myasthenic syndrome | CHRNE |
| Duchenne muscular dystrophy | DMD |
| Emery-Dreifuss muscular dystrophy, AD | LMNA |
| Limb-girdle muscular dystrophy 2A | CAPN3 |
| Limb-girdle muscular dystrophy, type 2D | SGCA |

TABLE 15
Skin

| Disease | Gene Affected |
|---|---|
| Epidermolysis Bullosa Dystrophica Recessive | COL7A1 |
| (Hallopeau-Siemens) | |
| Epidermolysis Bullosa Junctional | LAMB3 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

In some embodiments, a Gene Writer™ system described herein, or a component or portion thereof (e.g., a polypeptide or nucleic acid as described herein), is used to treat a genetic disease, disorder, or condition. In some embodiments, a Gene Writer™ system described herein, or a component or portion thereof (e.g., a polypeptide or nucleic acid as described herein), is used to treat a subject (e.g., a human patient) diagnosed with a genetic disease, disorder, or condition. In some embodiments, the genetic disease, disorder, or condition is associated with a specific genotype, e.g., a heterozygous or homozygous genotype. In some embodiments, the genetic disease, disorder, or condition is associated with a specific mutation, e.g., substitution, deletion, or insertion, e.g., a nucleotide expansion. In some embodiments, the genetic disease, disorder, or condition is cystic fibrosis or ornithine transcarbamylase (OTC) deficiency. In some embodiments, a Gene Writer™ system described herein for use in treating a genetic disease, disorder, or condition comprises a heterologous object sequence comprising a functional (e.g., wildtype) copy of a gene for which the subject (e.g., human patient) is deficient (e.g., wholly or in a target population of cells). In some embodiments, the functional copy of a gene comprises a functional (e.g., wildtype) CFTR gene or OTC gene.

In some embodiments, a Gene Writer™ system described herein, or a component or portion thereof (e.g., a polypeptide or nucleic acid as described herein), is used to treat a subject (e.g., human patient) having a biomarker (e.g., associated with a disease, disorder, or condition, e.g., a genetic disease, disorder, or condition) at a level outside of a healthy range. In some embodiments, a Gene Writer™ system described herein, or a component or portion thereof (e.g., a polypeptide or nucleic acid as described herein), is used to treat a subject (e.g., a human patient) diagnosed as having a biomarker (e.g., associated with a disease, disorder, or condition, e.g., a genetic disease, disorder, or condition) at a level outside of a healthy range.

In some embodiments, the presence and/or level of the biomarker and/or the genotype of the subject (e.g., human patient) is determined before treatment using a Gene Writer™ system described herein, or a component or portion thereof (e.g., a polypeptide or nucleic acid as described herein). In some embodiments, the presence and/or level of the biomarker and/or the genotype of the subject (e.g., human patient) is determined after treatment using a Gene Writer™ system described herein, or a component or portion thereof (e.g., a polypeptide or nucleic acid as described herein). In some embodiments, the presence and/or level of the biomarker and/or the genotype of the subject (e.g., human patient) is determined before and after treatment using a Gene Writer™ system described herein, or a component or portion thereof (e.g., a polypeptide or nucleic acid as described herein).

In some embodiments, a Gene Writer™ system described herein, or a component or portion thereof (e.g., a polypeptide or nucleic acid as described herein) is administered responsive to a determination that a biomarker is present at a level outside of a normal and/or healthy range in a subject (e.g., a human patient). In some embodiments, a Gene Writer™ system described herein, or a component or portion thereof (e.g., a polypeptide or nucleic acid as described herein) is re-administered responsive to a determination that a biomarker is present at a level outside of a normal and/or healthy range in a subject (e.g., a human patient) after a first administration of the Gene Writer™ system described herein, or a component or portion thereof. In some embodiments, a Gene Writer™ system described herein, or a component or portion thereof (e.g., a polypeptide or nucleic acid as described herein) is administered responsive to a determination that a subject (e.g., a human patient), e.g., or a target cell population in the subject, has a genotype (e.g., associated with a disease, disorder, or condition). In some embodiments, a Gene Writer™ system described herein, or a component or portion thereof (e.g., a polypeptide or nucleic acid as described herein) is re-administered responsive to a determination that a subject (e.g., a human patient), e.g., or a target cell population in the subject, has a genotype (e.g., associated with a disease, disorder, or condition) after a first administration of the Gene Writer™ system described herein, or a component or portion thereof. In some embodiments, administration of a Gene Writer™ system described herein, or a component or portion thereof (e.g., a polypeptide or nucleic acid as described herein) continues or is repeated until a biomarker is present at a level within a normal and/or healthy range in the subject (e.g., a human patient). In some embodiments, administration of a Gene Writer™ system described herein, or a component or portion thereof (e.g., a polypeptide or nucleic acid as described herein) continues or is repeated until the subject (e.g., a human patient), e.g., or a target cell population in the subject, does not have the genotype (e.g., associated with a disease, disorder, or condition).

In some embodiments, a Gene Writer™ system described herein, or a component or portion thereof (e.g., a polypeptide or nucleic acid as described herein), is used to treat a disease, disorder, or condition prenatally (e.g., in a human subject in utero, e.g., an embryo or fetus). In some embodiments, a Gene Writer™ system described herein, or a component or portion thereof (e.g., a polypeptide or nucleic acid as described herein), is used to treat a disease, disorder, or condition postnatally, e.g., in a human infant, toddler, or child. In some embodiments, a Gene Writer™ system described herein, or a component or portion thereof (e.g., a polypeptide or nucleic acid as described herein), is used to treat a disease, disorder, or condition neonatally.

In some embodiments, the genotype of a subject (e.g., a human patient), e.g., or a target cell population in the subject, treated with a Gene Writer™ system described herein, or a component or portion thereof (e.g., a polypeptide or nucleic acid as described herein), remains stable as the subject develops. Stable in this context may refer to the absence of additional alterations in a subject's genotype (e.g., or a target cell population in the subject) after treatment with the Gene Writer™ system described herein, or a component or portion thereof (e.g., a polypeptide or nucleic acid as described herein) is complete. Stable in this context may additionally or alternatively refer to the persistence of an alteration to the subject's genotype made by a Gene Writer system described herein. Without wishing to be bound by theory, it may be desirable to avoid, prevent, or minimize additional alterations in the genotype of a subject besides those made by the Gene Writer system. Additionally or alternatively, it may be desirable that the alteration of the genotype of a subject (e.g., or a target cell population in the subject), persist after completion of treatment (e.g., for at least a selected time interval, e.g., indefinitely). In some embodiments, the genotype of a subject, e.g., or a target cell population in the subject, after the completion of treatment is the same as the genotype of the subject, e.g., or the target cell population in the subject, at a selected time interval after treatment, e.g., 1, 2, 3, 4, 5, 6, or 7 days, or 1, 2, 3, 4, 5, 6, 7, 8, 9, or 10 weeks, or 3, 4, 5, 6, 7, 8, 9, 10, or 11 months, or 1, 2, 3, 4, 5, 6, 7, 8, 9, or 10 years (e.g., indefinitely). In some embodiments, an alteration to the genotype of a subject, e.g., or a target cell population in the subject, made by the Gene Writer™ system described herein, or a component or portion thereof (e.g., a polypeptide or nucleic acid as described herein) persists for at least a selected time interval after treatment, e.g., 1, 2, 3, 4, 5, 6, or 7 days, or 1, 2, 3, 4, 5, 6, 7, 8, 9, or 10 weeks, or 3, 4, 5, 6, 7, 8, 9, 10, or 11 months, or 1, 2, 3, 4, 5, 6, 7, 8, 9, or 10 years (e.g., indefinitely).

### Plant-Modification Methods

Gene Writer systems described herein may be used to modify a plant or a plant part (e.g., leaves, roots, flowers, fruits, or seeds), e.g., to increase the fitness of a plant.

A. Delivery to a Plant

Provided herein are methods of delivering a Gene Writer system described herein to a plant. Included are methods for delivering a Gene Writer system to a plant by contacting the plant, or part thereof, with a Gene Writer system. The methods are useful for modifying the plant to, e.g., increase the fitness of a plant.

More specifically, in some embodiments, a nucleic acid described herein (e.g., a nucleic acid encoding a GeneWriter) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime-editing systems for precise genome editing, 2020, Plant Communications.

In one aspect, provided herein is a method of increasing the fitness of a plant, the method including delivering to the plant the Gene Writer system described herein (e.g., in an effective amount and duration) to increase the fitness of the plant relative to an untreated plant (e.g., a plant that has not been delivered the Gene Writer system).

An increase in the fitness of the plant as a consequence of delivery of a Gene Writer system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2×-fold, 5×-fold, 10×-fold, 25×-fold, 50×-fold, 75×-fold, 100×-fold, or more than 100×-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a Gene Writer system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents.

Accordingly, provided herein is a method of modifying a plant, the method including delivering to the plant an effective amount of any of the Gene Writer systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some s 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the Gene Writer systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the Gene Writer systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the Gene Writer system described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

B. Application Methods

A plant described herein can be exposed to any of the Gene Writer system compositions described herein in any suitable manner that permits delivering or administering the composition to the plant. The Gene Writer system may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the plant-modifying composition. Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the Gene Writer system is delivered to a plant, the plant receiving the Gene Writer system may be at any stage of plant growth. For example, formulated plant-modifying compositions can be applied as a seed-coating or root treatment in early stages of plant growth or as a total plant treatment at later stages of the crop cycle. In some instances, the plant-modifying composition may be applied as a topical agent to a plant.

Further, the Gene Writer system may be applied (e.g., in the soil in which a plant grows, or in the water that is used to water the plant) as a systemic agent that is absorbed and distributed through the tissues of a plant. In some instances, plants or food organisms may be genetically transformed to express the Gene Writer system.

Delayed or continuous release can also be accomplished by coating the Gene Writer system or a composition with the plant-modifying composition(s) with a dissolvable or bioerodable coating layer, such as gelatin, which coating dissolves or erodes in the environment of use, to then make the plant-modifying com Gene Writer system position available, or by dispersing the agent in a dissolvable or erodible matrix. Such continuous release and/or dispensing means devices may be advantageously employed to consistently maintain an effective concentration of one or more of the plant-modifying compositions described herein.

In some instances, the Gene Writer system is delivered to a part of the plant, e.g., a leaf, seed, pollen, root, fruit, shoot, or flower, or a tissue, cell, or protoplast thereof. In some instances, the Gene Writer system is delivered to a cell of the plant. In some instances, the Gene Writer system is delivered to a protoplast of the plant. In some instances, the Gene Writer system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the Gene Writer system is delivered to a plant embryo.

C. Plants

A variety of plants can be delivered to or treated with a Gene Writer system described herein. Plants that can be delivered a Gene Writer system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the Gene Writer system is delivered to a plant part, the plant part may be modified by the plant-modifying agent. Alternatively, the Gene Writer system may be distributed to other parts of the plant (e.g., by the plant's circulatory system) that are subsequently modified by the plant-modifying agent.

### Lipid Nanoparticles

The methods and systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the Gene Writer or template nucleic acid) can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

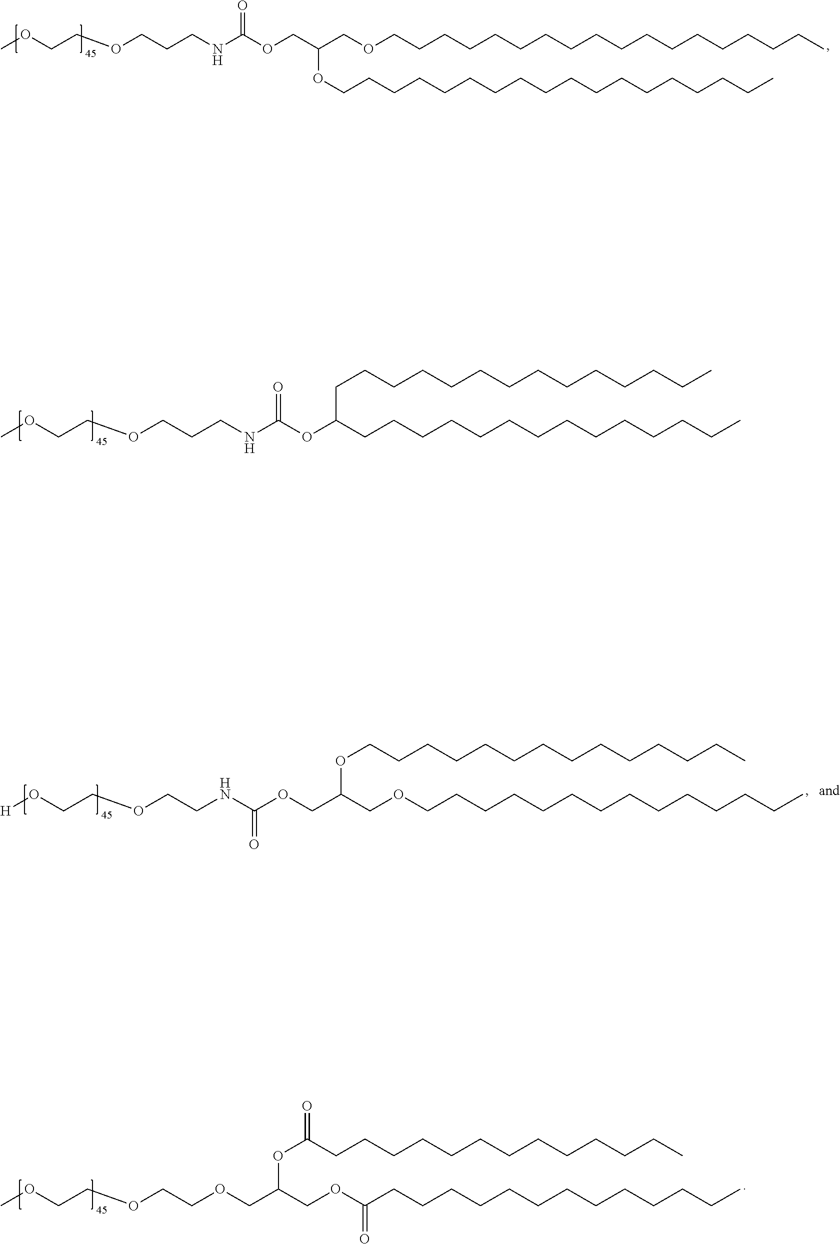

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a Gene Writer system may be prepared as a single LNP formulation, e.g., an LNP formulation comprises mRNA encoding for the Gene Writer polypeptide and an RNA template. Ratios of nucleic acid components may be varied in order to maximize the properties of a therapeutic. In some embodiments, the ratio of RNA template to mRNA encoding a Gene Writer polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a Gene Writer polypeptide. In some embodiments, the system may comprise more than two nucleic acid components formulated into LNPs. In some embodiments, the system may comprise a protein, e.g., a Gene Writer polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., Gene Writer polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of Gene Writer LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 10¹⁴ vg/kg.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jul. 19, 2019. When one gene or protein references a plurality of sequence accession numbers, all of the sequence variants are encompassed.

### EXAMPLES

The invention is further illustrated by the following examples. The examples are provided for illustrative purposes only and are not to be construed as limiting the scope or content of the invention in any way.

### Example 1: Delivery of a Gene Writer™ System to Mammalian Cells

This example describes a Gene Writer™ genome editing system delivered to a mammalian cell for site-specific insertion of exogenous DNA into a mammalian cell genome.

In this example, the polypeptide component of the Gene Writer™ system is a recombinase protein selected from Table 1, column 1, and the template DNA component is a plasmid DNA that comprises a target recombination site, e.g., as listed in a corresponding row of Table 1.

HEK293T cells are transfected with the following test agents:
1. Scrambled DNA control
2. DNA coding for the polypeptide described above
3. Template DNA described above
4. Combination of 2 and 3

After transfection, HEK293T cells are cultured for at least 4 days and then assayed for site-specific genome editing. Genomic DNA is isolated from each group of HEK293 cells. PCR is conducted with primers that flank the appropriate genomic locus selected from Table 1 column 4. The PCR product is run on an agarose gel to measure the length of the amplified DNA.

A PCR product of the expected length, indicative of a successful Gene Writing™ genome editing event that inserts the DNA plasmid template into the target genome, is observed only in cells that were transfected with the complete Gene Writer™ system of group 4 above.

### Example 2: Targeted Delivery of a Gene Expression Unit into Mammalian Cells Using a Gene Writer™ System

This example describes the making and using of a Gene Writer genome editor to insert a heterologous gene expression unit into the mammalian genome.

In this example, a recombinase protein is selected from Table 1, column 1. The recombinase protein targets the corresponding genomic locus listed in column 4 of Table 1 for DNA integration. The template DNA component is a plasmid DNA that comprises a target recombination site and gene expression unit. A gene expression unit comprises at least one regulatory sequence operably linked to at least one coding sequence. In this example, the regulatory sequences include the CMV promoter and enhancer, an enhanced translation element, and a WPRE. The coding sequence is the GFP open reading frame.

HEK293 cells are transfected with the following test agents:
1. Scrambled DNA control
2. DNA coding for the polypeptide described above
3. Template DNA described above
4. Combination of 2 and 3

After transfection, HEK293 cells are cultured for at least 4 days and assayed for site-specific Gene Writing genome editing. Genomic DNA is isolated from the HEK293 cells and PCR is conducted with primers that flank the target integration site in the genome. The PCR product is run on an agarose gel to measure the length of DNA. A PCR product of the expected length, indicative of a successful Gene Writing™ genome editing event, is detected in cells transfected with the test agent of group 4 (complete Gene Writer™ system).

The transfected cells are cultured for a further 10 days, and after multiple cell culture passages are assayed for GFP expression via flow cytometry. The percent of cells that are GFP positive from each cell population are calculated. GFP positive cells are detected in the population of HEK293 cells that were transfected with group 4 test agent, demonstrating that a gene expression unit added into the mammalian cell genome via Gene Writing genome editing is expressed.

### Example 3: Targeted Delivery of a Splice Acceptor Unit into Mammalian Cells Using a Gene Writer™ System

This example describes the making and use of a Gene Writing genome editing system to add a heterologous sequence into an intronic region to act as a splice acceptor for an upstream exon. Splicing into the first intron a new exon containing a splice acceptor site at the 5′ end and a polyA tail at the 3′ end will result in a mature mRNA containing the first natural exon of the natural locus spliced to the new exon.

In this example, a recombinase protein selected from Table 1, column 1. The recombinase protein targets the corresponding genomic locus listed in Table 1, column 4, for DNA integration. The template DNA codes for GFP with a splice acceptor site immediately 5′ to the first amino acid of mature GFP (the start codon is removed) and a 3′ polyA tail downstream of the stop codon.

HEK293 cells are transfected with the following test agents:
1. Scrambled DNA control
2. DNA coding for the polypeptide described above
3. Template DNA described above
4. Combination of 2 and 3

After transfection, HEK293 cells are cultured for at least 4 days and assayed for site-specific Gene Writing genome editing and appropriate mRNA processing. Genomic DNA is isolated from the HEK293 cells. Reverse transcription-PCR is conducted to measure the mature mRNA containing the first natural exon of the target locus and the new exon. The RT-PCR reaction is conducted with forward primers that bind to the first natural exon of the target locus and with reverse primers that bind to GFP. The RT-PCR product is run on an agarose gel to measure the length of DNA. A PCR product of the expected length is detected in cells transfected with the test agent of group 4, indicative of a successful Gene Writing genome editing event and a successful splice event. This result would demonstrate that a Gene Writing genome editing system can add a heterologous sequence encoding a gene into an intronic region to act as a splice acceptor for the upstream exon.

The transfected cells are cultured for a further 10 days and, after multiple cell culture passages, are assayed for GFP expression via flow cytometry. The percent of cells that are GFP positive from each cell population are calculated. GFP positive cells are detected in the population of HEK293 cells that were transfected with group 4 test agent, demonstrating that a gene expression unit added into the mammalian cell genome via Gene Writing genome editing is expressed.

### Example 4: Specificity of Gene Writing in Mammalian Cells

This example describes a Gene Writer™ genome system delivered to a mammalian cell for site-specific insertion of exogenous DNA into a mammalian cell genome and a measurement of the specificity of the site-specific insertion.

In this example, Gene Writing is conducted in HEK293T cells as described in any of the preceding Examples. After transfection, HEK293T cells are cultured for at least 4 days and then assayed for site-specific genome editing. Linear amplification PCR is conducted as described in Schmidt et al. Nature Methods 4, 1051-1057 (2007) using a forward primer specific to the template DNA that will amplify adjacent genomic DNA. Amplified PCR products are then sequenced using next generation sequencing technology on a MiSeq instrument. The MiSeq reads are mapped to the HEK293T genome to identify integration sites in the genome.

The percent of LAM-PCR sequencing reads that map to the target genomic site is the specificity of the Gene Writer.

The number of total genomic sites that LAM-PCR sequencing reads map to is the number of total integration sites.

### Example 5: Efficiency of Gene Writing in Mammalian Cells

This example describes Gene Writer™ genome system delivered to a mammalian cell for site-specific insertion of exogenous DNA into a mammalian cell genome, and a measurement of the efficiency of Gene Writing.

In this example, Gene Writing is conducted in HEK293T cells as described in any of the preceding Examples. After transfection, HEK293T cells are cultured for at least 4 days and then assayed for site-specific genome editing. Digital droplet PCR is conducted as described in Lin et al., Human Gene Therapy Methods 27(5), 197-208, 2016. A forward primer binds to the template DNA and a reverse primer binds on one side of the appropriate genomic locus selected from Table 1 column 4, thus a PCR amplification is only expected upon integration of target DNA. A probe to the target site containing a FAM fluorophore and is used to measure the number of copies of the target DNA in the genome. Primers and HEX-fluorophore probe specific to a housekeeping gene (e.g. RPP30) are used to measure the copies of genomic DNA per droplet.

The copy number of target DNA per droplet normalized to the copy number of house keeping DNA per droplet is the efficiency of the Gene Writer.

### Example 6: Determination of Copy Number of a Recombinase in a Cell

The following example describes the absolute quantification of a recombinase on a per cell basis. This measurement is performed using the AQUA mass spectrometry based methods, e.g., as accessible at the following uniform resource locator (URL):https://www.sciencedirect.com/science/article/pii/S1046202304002087?via%3Dihub

Following delivery of the recombinase and DNA template to the cells, the recombination is allowed to proceed for 24 hours after which the cells are quantified and then quantified by this MS method. This method involves two stages.

In the first stage, the amino acid sequence of the recombinase is examined, and a representative tryptic peptide is selected for analysis. An AQUA peptide is then synthesized with an amino acid sequence that exactly mimics the corresponding native peptide produced during proteolysis. However, stable isotopes are incorporated at one residue to allow the mass spectrometer to differentiate between the analyte and internal standard. The synthetic peptide and the native peptide share the same physicochemical properties including chromatographic co-elution, ionization efficiency, and relative distributions of fragment ions, but are differentially detected in a mass spectrometer due to their mass difference. The synthetic peptide is next analyzed by LC-MS/MS techniques to confirm the retention time of the peptide, determine fragment ion intensities, and select an ion for SRM analysis. In such an SRM experiment, a triple quadrupole mass spectrometer is directed to select the expected precursor ion in the first scanning quadrupole, or Q1. Only ions with this one mass-to-charge (m/z) ratio are directed into the collision cell (Q2) to be fragmented. The resulting product ions are passed to the third quadrupole (Q3), where the m/z ratio for single fragment ion is monitored across a narrow m/z window.

The second stage involves quantification of the recombinase from cell or tissue lysates. A quantified number of cells or mass of tissue is used to initiate the reaction and is used to normalize the quantification to a per cell basis. Cell lysates are separated prior to proteolysis to increase the dynamic range of the assay via SDS-PAGE, followed by excision of the region of the gel where the recombinase migrates. In-gel digestion is performed to obtain native tryptic peptides. In-gel digestion is performed in the presence of the AQUA peptide, which is added to the gel pieces during the digestion process. Following proteolysis, the complex peptide mixture, containing both heavy and light peptides, is analyzed in an LC-SRM experiment using parameters determined during the first stage.

The results of the mass spectrometry-based quantification is converted to a number of proteins loaded to determine the number of recombinases per cell.

### Example 7: Copy Number of DNA Inside Cell

### Q-FISH

The following example describes the quantification of delivered DNA template on a per cell basis. In this example the DNA that the recombinase is integrating contains a DNA-probe binding site. Following delivery of the recombinase and DNA template to the cells, the recombination is allowed to proceed for 24 hours, after which the cells are quantified and are prepared for quantitative fluorescence in situ hybridization (Q-FISH). Q-FISH is conducted using FISH Tag DNA Orange Kit, with Alex Fluor 555 dye (ThermoFisher catalog number F32948). Briefly, a DNA probe that binds to the DNA-probe binding site on the DNA template is generated through a procedure of nick translation, dye labeling, and purification as described in the Kit manual. The cells are then labeled with the DNA probe as described in the Kit manual. The cells are imaged on a Zeiss LSM 710 confocal microscope with a 63× oil immersion objective while maintained at 37 C and 5% CO2. The DNA probe is subjected to 555 nm laser excitation to stimulate Alexa Flour. A MATLAB script is written to measure the Alex Fluor intensity relative to a standard generated with known quantities of DNA. Using this method, the amount of template DNA delivered to a cell is determined.

qPCR

The following example describes the quantification of delivered DNA template on a per cell basis. In this example the DNA that the recombinase is integrating contains a DNA-probe binding site. Following delivery of the recombinase and DNA template to the cells, the recombination is allowed to proceed for 24 hours after which the cells are quantified, and cells are prepared for quantitative PCR (qPCR). qPCR is conducted using standard kits for this protocol, such as the ThermoFisher TaqMan product (https://www.thermofisher.com/us/en/home/life-science/pcr/real-time-pcr/real-time-pcr-assays-search.html). Briefly, primers are designed that specifically amplify a region of the delivered template DNA as well as probes for the specific amplicon. A standard curve is generated by using a serial dilution of quantified pure template DNA to correlate threshold Ct numbers to number of DNA templates. The DNA is then extracted from the cells being analyzed and input into the qPCR reaction along with all additional components per the manufacturer's directions. The samples are than analyzed on an appropriate qPCR machine to determine the Ct number, which is then mapped to the standard curve for absolute quantification. Using this method, the amount of template DNA delivered to a cell is determined.

### Example 8: Intracellular Ratio of DNA: Recombinase

The following example describes the determination of the ratio of recombinase protein to template DNA cell in the target cells. Following delivery of the recombinase and DNA template to the cells, the recombination is allowed to proceed for 24 hours after which the cells are quantified, and cells are prepared quantification of the recombinase and of the template DNA as outlined in the above examples. These two values (recombinase per cell and template DNA per cell) are then divided (recombinase per cell/template DNA per cell) to determine the bulk average ratio of these quantities. Using this method, the ratio of recombinase to template DNA delivered to a cell is determined.

### Example 9: Activity in Presence of DNA-Damage Response Inhibiting Agents—Activity in Presence of NHEJ Inhibitor

The following example describes the assaying of activity of the recombinase protein in the presence of inhibitors of non-homologous end joining to highlight the lack of dependence on the expression of the proteins involved in these pathways for activity of the recombinase. Briefly, the assay outlined to determine efficiency of recombinase activity outlined in the example above is performed. However, in this case two separate experiments are performed.

In experiment 1, 24 hours after delivery of the recombinase and Template DNA, 1 μM of the NHEJ inhibitor Scr7 (https://www.sigmaaldrich.com/catalog/product/sigma/sml1546?lang=en&region=US) is added to the cell growth media to inhibit this pathway. All other elements of the protocol are identical.

In experiment 2, the cells are manipulated identically as in experiment 1 but no inhibitor is added to the media. Both experiments are analyzed for efficiency per the example above and the % inhibited activity relative to uninhibited activity is determined.

### Example 10: Activity in Presence of DNA-Damage Response Inhibiting Agents—Activity in Presence of HDR Inhibitor

The following example describes the assaying of activity of the recombinase protein in the presence of inhibitors of homologous recombination to highlight the lack of dependence on the expression of the proteins involved in these pathways for activity of the recombinase. Briefly, the assay outlined to determine efficiency of recombinase activity outlined in the example above is performed. However, in this case, two separate experiments are performed.

In experiment 1: 24 hours after delivery of the recombinase and Template DNA, 1 μM of the HR inhibitor B02 (https://www.selleckchem.com/products/b02.html) is added to the cell growth media to inhibit this pathway. All other elements of the protocol are identical.

In experiment 2: the cells are manipulated identically as in experiment 1 but no inhibitor is added to the media. Both experiments are analyzed for efficiency per the example above and the % inhibited activity relative to uninhibited activity is determined.

### Example 11: Percentage of Nuclear Versus Cytoplasmic Recombinase

The following example describes the determination of the ratio of recombinase protein in the nucleus vs the cytoplasm of target cells. 12 hours following delivery of the recombinase and DNA template to the cells as described herein, the cells are quantified and prepared for analysis. The cells are split into nuclear and cytoplasmic fractions using the following standard kits, following manufacturer directions: NE-PER Nuclear and Cytoplasmic Extraction by ThermoFisher. Both the cytoplasmic and nuclear fractions are kept and then put through the mass spec based recombinase quantification assay outlined in the example above. Using this method, the ratio of nuclear recombinase to cytoplasmic recombinase in the cells is determined.

### Example 12: Delivery to Plant Cells

This example illustrates a method of delivering at least one recombinase to a plant cell wherein the plant cell is located in a plant or plant part. More specifically, this example describes delivery of a Gene Writing recombinase and its template DNA to a non-epidermal plant cell (i.e., a cell in a soybean embryo), in order to edit an endogenous plant gene (i.e., phytoene desaturase, PDS) in germline cells of excised soybean embryos. This example describes delivery of polynucleotides encoding the delivered transgene through multiple barriers (e.g., multiple cell layers, seed coat, cell walls, plasma membrane) directly into soybean germline cells, resulting in a heritable alteration of the target nucleotide sequence, PDS. The methods described do not employ the common techniques of bacterially mediated transformation (e.g., by Agrobacterium sp.) or biolistics.

Plasmids are designed for delivery of recombinase and a single template DNA targeting the endogenous phytoene desaturase (PDS) in soybean (Glycine max). It will be apparent to one skilled in the art that analogous plasmids are easily designed to encode other recombinases and template DNA sequences, optionally including different elements (e. g., different promoters, terminators, selectable or detectable markers, a cell-penetrating peptide, a nuclear localization signal, a chloroplast transit peptide, or a mitochondrial targeting peptide, etc.), and used in a similar manner.

In a first series of experiments, these vectors are delivered to non-epidermal plant cells in soybean embryos using combinations of delivery agents and electroporation. Mature, dry soybean seeds (cv. Williams 82) are surface-sterilized as follows. Dry soybean seeds are held for 4 hours in an enclosed chamber holding a beaker containing 100 milliliters 5% sodium hypochlorite solution to which 4 milliliters hydrochloric acid are freshly added. Seeds remain desiccated after this sterilization treatment. The sterilized seeds are split into 2 halves by manual application of a razor blade and the embryos are manually separated from the cotyledons. Each test or control treatment is carried out on 20 excised embryos. The following series of experiments is then performed.

Experiment 1: A delivery solution containing the vectors (100 nanograms per microliter of each plasmid) in 0.01% CTAB (cetyltrimethylammonium bromide, a quaternary ammonium surfactant) in sterile-filtered milliQ water is prepared. Each solution is chilled to 4 degrees Celsius and 500 microliters are added directly to the embryos, which are then immediately placed on ice in a vacuum chamber and subjected to a negative pressure (2×10″3 millibar) treatment for 15 minutes. Following the chilling/negative pressure treatments, the embryos are treated with electric current using a BTX-Harvard ECM-830 electroporation device set with the following parameters: 50V, 25 millisecond pulse length, 75 millisecond pulse interval for 99 pulses.
Experiment 2: conditions identical to Experiment 1, except that the initial contacting with delivery solution and negative pressure treatments are carried out at room temperature.
Experiment 3: conditions identical to Experiment 1, except that the delivery solution is prepared without CTAB but includes 0.1% Silwet L-77™ (CAS Number 27306-78-1, available from Momentive Performance Materials, Albany, N.Y). Half (10 of 20) of the embryos receiving each treatment undergo electroporation, and the other half of the embryos do not.
Experiment 4: conditions identical to Experiment 3, except that several delivery solutions are prepared, where each further includes 20 micrograms/milliliter of one single-walled carbon nanotube preparation selected from those with catalogue numbers 704113, 750530, 724777, and 805033, all obtainable from Sigma-Aldrich, St. Louis, Mo. Half (10 of 20) of the embryos receiving each treatment undergo electroporation, and the other half of the embryos do not.
Experiment 5: conditions identical to Experiment 3, except that the delivery solution further includes 20 micrograms/milliliter of triethoxylpropylaminosilane-functionalized silica nanoparticles (catalogue number 791334, Sigma-Aldrich, St. Louis, Mo. Half (10 of 20) of the embryos receiving each treatment undergo electroporation, and the other half of the embryos do not.
Experiment 6: conditions identical to Experiment 3, except that the delivery solution further includes 9 micrograms/milliliter branched polyethylenimine, molecular weight −25,000 (CAS Number 9002-98-6, catalogue number 408727, Sigma-Aldrich, St. Louis, Mo.) or 9 micro grams/milliliter branched polyethylenimine, molecular weight −800 (CAS Number 25987-06-8, catalogue number 408719, Sigma-Aldrich, St. Louis, Mo.). Half (10 of 20) of the embryos receiving each treatment undergo electroporation, and the other half of the embryos do not.
Experiment 7: conditions identical to Experiment 3, except that the delivery solution further includes 20% v/v dimethylsulf oxide (DMSO, catalogue number D4540, Sigma-Aldrich, St. Louis, Mo.). Half (10 of 20) of the embryos receiving each treatment undergo electroporation, and the other half of the embryos do not.
Experiment 8: conditions identical to Experiment 3, except that the delivery solution further contains 50 micromolar nono-arginine (RRRRRRRRR, SEQ ID NO:1873). Half (10 of 20) of the embryos receiving each treatment undergo electroporation, and the other half of the embryos do not.
Experiment 9: conditions identical to Experiment 3, except that following the vacuum treatment, the embryos and treatment solutions are transferred to microcentrifuge tubes and centrifuged 2, 5, 10, or 20 minutes at 4000×g. Half (10 of 20) of the embryos receiving each treatment undergo electroporation, and the other half of the embryos do not.
Experiment 10: conditions identical to Experiment 3, except that following the vacuum treatment, the embryos and treatment solutions are transferred to microcentrifuge tubes and centrifuged 2, 5, 10, or 20 minutes at 4000×g.
Experiment 11: conditions identical to Experiment 4, except that following the vacuum treatment, the embryos and treatment solutions are transferred to microcentrifuge tubes and centrifuged 2, 5, 10, or 20 minutes at 4000×g.
Experiment 12: conditions identical to Experiment 5, except that following the vacuum treatment, the embryos and treatment solutions are transferred to microcentrifuge tubes and centrifuged 2, 5, 10, or 20 minutes at 4000×g.

After the delivery treatment, each treatment group of embryos is washed 5 times with sterile water, transferred to a petri dish containing ½ MS solid medium (2.165 g Murashige and Skoog medium salts, catalogue number MSP0501, Caisson Laboratories, Smithfield, Utah), 10 grams sucrose, and 8 grams Bacto agar, made up to 1.00 liter in distilled water), and placed in a tissue culture incubator set to 25 degrees Celsius. After the embryos have elongated, developed roots and true leaves have emerged, the seedlings are transferred to soil and grown out. Modification of all endogenous PDS alleles results in a plant unable to produce chlorophyll and having a visible bleached phenotype. Modification of a fraction of all endogenous PDS alleles results in plants still able to produce chlorophyll; plants that are heterozygous for an altered PDS gene will are grown out to seed and the efficiency of heritable genome modification is determined by molecular analysis of the progeny seeds.

### Example 13: Assessment of Gene Writer Activity in Human Cells by Episomal Reporter Inversion Assay

This example describes a reporter assay for Gene Writer activity in human cells. Specifically, the reporter assay involves the co-delivery of an inactive reporter plasmid and a second plasmid bearing a tyrosine recombinase that may activate an inverted GFP gene on the reporter plasmid.

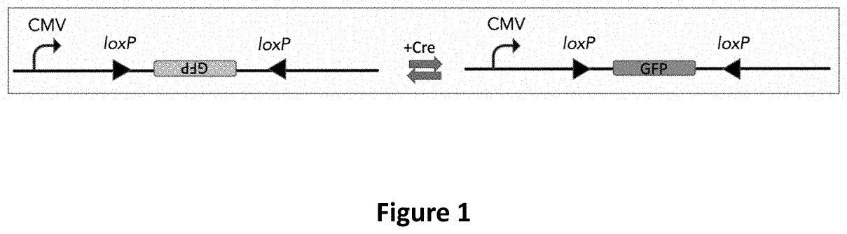

In this example, a Gene Writer and a reporter were delivered to HEK293T cells. The delivery comprised two plasmids: 1) the recombinase expression plasmid encoding a recombinase sequence (e.g., a recombinase from Table 1, recombinase sequence from Table 2) driven by the mammalian CMV promoter, and 2) the reporter plasmid comprising a CMV promoter upstream of a recombinase target site flanked inverted EGFP sequence (e.g., an inverted EGFP sequence flanked by a pair of recognition sites from Column 2 or 3 of Table 1, in inverted orientation relative to each other). Tyrosine recombinases that were discovered as described elsewhere herein and that recognize palindromic sequences with homology to the human genome, comprising up to 3 mismatches, were selected for activity testing on both their natural sequences (e.g., natural sequences as discovered in bacteria, e.g., as describe in Column 2 of Table 1) as well as the corresponding human genome sequence (containing up to 3 mismatches, e.g., as described in Column 3 of Table 1). The presence of a cognate recombinase results in inversion of the EGFP sequence and allows EGFP expression driven by the CMV promoter, e.g., as shown in the schematic in FIG. 1.

Approximately 120,000 HEK293T cells were either co-transfected with recombinase expressing plasmid and inverted GFP reporter plasmid at a 1:3 recombinase:reporter plasmid molar ratio using TransIT-293 Reagent (Mirusbio), or transfected similarly with reporter plasmid alone as a negative control. Two days after transfection, recombinase activity was measured using flow cytometry to determine the percentage of EGFP positive cells. Results of flow cytometry analysis are provided in Table 16, and show that a recombinase with activity in human cells resulted in an increase in the percentage of EGFP positive cells over the negative control (reporter plasmid only).

### Example 14: Assessment of Gene Writer Activity in Human Cells by Integration at Endogenous Genomic Loci

This example describes an integration assay for Gene Writer activity in human cells. Specifically, the assay involves the co-delivery of an insert DNA plasmid comprising a heterologous object sequence and a recombinase recognition site and a second plasmid bearing a tyrosine recombinase for catalyzing the integration of the insert DNA plasmid into the genome.

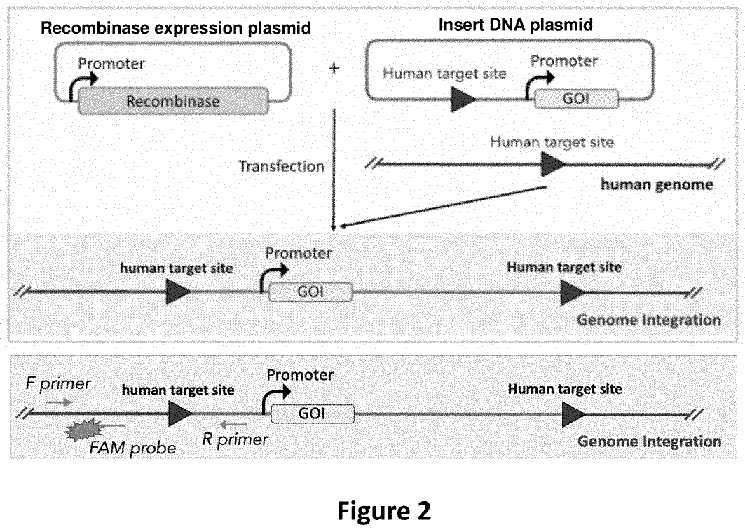

In this example, a Gene Writer and a sequence of interest were delivered to HEK293T cells. The delivery comprised two plasmids: 1) the recombinase expression plasmid harboring a recombinase sequence (e.g., a recombinase from Table 1, recombinase sequence from Table 2) driven by the mammalian CMV promoter, and 2) the insert DNA plasmid comprising a CMV promoter upstream of a gene of interest (e.g., a GFP sequence) and a native recombinase recognition site (e.g., a sequence of Column 2 of Table 1) or a recombinase recognition site matching a sequence in the human genome, e.g., a sequence in the human genome with homology to the native recognition site (e.g., a sequence of Column 3 of Table 1), with three or fewer mismatches. An example integration reaction is shown in FIG. 2.

Approximately 120,000 HEK293T cells were either co-transfected with recombinase expressing plasmid and insert DNA plasmid at a 1:3 recombinase:insert DNA plasmid molar ratio using TransIT-293 Reagent (Mirusbio), or transfected similarly with reporter plasmid alone as a negative control. At 2-5 days post-transfection, recombinase-mediated genome integration was measured using Droplet Digital PCR (ddPCR). The percentage of cells undergoing successful integration was approximated by calculating the average genomic copy number of insert DNA integrants normalized to an RPP30 reference control. Results of ddPCR analysis are provided in Table 16, and shows that a recombinase able to integrate the insert DNA plasmid into the human genome resulted in an increase in the average number of integration events per genome over the negative control (reporter plasmid only).

### Example 15: Inversion and Integration Assay Data

Recombinases from Table 1 or 2 were tested in human cells using an episomal reporter inversion (Example 13) or genomic integration (Example 14) assay and the data is shown in Table 16. Column 2 indicates the accession of recombinase proteins as listed in Tables 1 and 2. For the episomal assay, inversion activity is shown as % of GFP+ cells as measured by flow cytometry, where Column 4 indicates inversion activity using the natural recognition sites (Column 2 of Table 1) and Column 6 indicates inversion activity using the closest matching human site (Column 3 of Table 1), with Columns 3 and 5 displaying the respective background GFP in the absence of recombinase. For the genomic integration assay, integration activity measured by ddPCR is expressed as % of cells estimated by the average copies of integrated insert DNA vector per genome copy and is shown in Column 7. Of the exemplary recombinases listed in Table 16, at least 34 showed activity above background using the closest matching human site in the episomal reporter inversion assay. Of these, at least 21 showed activity that was at least twice the background level using the closest matching human site. Of the exemplary recombinases listed in Table 16 that were tested by genomic integration assay, at least 17 showed activity at the closest matching site in the human genome. NT=Not Tested

TABLE 16
Recombinase activity in human cells.

| | | 3. | 4. | 5. | 6. | |
|---|---|---|---|---|---|---|
| 1. | 2. | GFP Neg | GFP+ Rec | GFP Neg | GFP+ Rec | 7. |
| Recombinase | Protein_ID | (Natural) | (Natural) | (Human) | (Human) | Integration |
| Rec1 | WP_010497271.1 | 8.22 | 9.77 | 7.25 | 7.965 | NT |
| Rec2 | WP_006717173.1 | 0.2565 | 0.133 | 2.27 | 0.88 | NT |
| Rec3 | WP_006718580.1 | 0.2565 | 0.096 | 2.27 | 0.75 | NT |
| Rec4 | WP_006719234.1 | 0.2565 | 0.1335 | 2.27 | 0.55 | NT |
| Rec5 | WP_109859198.1 | 0.265 | 0.195 | 2.27 | 0.545 | NT |
| Rec2 | WP_006717173.1 | 0.265 | 0.27 | 2.27 | 0.76 | NT |
| Rec6 | WP_006717195.1 | 0.2565 | 0.135 | 2.27 | 0.705 | NT |
| Rec7 | WP_005715799.1 | 0.2565 | 0.108 | 2.27 | 0.78 | NT |
| Rec8 | WP_017740000.1 | 0.264 | 0.1645 | 0.165 | 0.047 | NT |
| Rec9 | WP_017744257.1 | 0.264 | 0.1325 | 0.165 | 0.05 | NT |
| Rec10 | WP_017746151.1 | 0.264 | 0.1675 | 0.165 | 0.047 | NT |
| Rec11 | WP_038150996.1 | 0.07465 | 0.02366 | 1.8875 | 3.065 | NT |
| Rec12 | WP_038150898.1 | 0.07465 | 0.031 | 1.8875 | 2.715 | NT |
| Rec13 | WP_126045042.1 | 5.795 | 4.465 | 4.085 | 9.235 | NT |
| Rec14 | WP_061329756.1 | 1.04 | 1.18 | 4.085 | 1.715 | NT |
| Rec15 | XP_012333305.1 | 2.178 | 5.905 | 3.435 | 7.24 | NT |
| Rec16 | WP_120166565.1 | 4.755 | 7.985 | 6.21 | 11.42 | NT |
| Rec17 | WP_073025039.1 | 4.4 | 8.625 | 3.355 | 68.4 | 0.15 |
| Rec18 | WP_007635552.1 | 1.255 | 0.202 | 3.355 | 1.045 | NT |
| Rec19 | WP_058958135.1 | 0.0065 | 63.65 | 6.21 | 54.8 | 0.86 |
| Rec20 | WP_090967054.1 | 4.93 | 70.65 | 6.21 | 63.55 | 0.54 |
| Rec21 | WP_010365336.1 | 4.91 | 4.75 | 3.355 | 0.98 | NT |
| Rec22 | WP_016392893.1 | 1.66 | 1.45 | 10.985 | 11.49 | NT |
| Rec23 | WP_047824597.1 | 0.81 | 43.3 | 0.188665 | 9.96 | 0 |
| Rec24 | WP_046407494.1 | 2.34 | 17.1 | 6.505 | 11.7 | NT |
| Rec25 | WP_003712523.1 | 3.3 | 4.845 | 0.1935 | 0.275 | NT |
| Rec26 | WP_005027658.1 | 3.98 | 4.115 | 1.475 | 2.05 | NT |
| Rec27 | WP_021170377.1 | 6.51 | 62.7 | 1.2395 | 45.2 | 0.87 |
| Rec28 | WP_015169902.1 | 6.76 | 10.165 | 3.705 | 4.62 | NT |
| Rec29 | WP_089415106.1 | 1.305 | 36.85 | 2.215 | 30.9 | 0.28 |
| Rec30 | WP_022624268.1 | 1.305 | 32.1 | 2.215 | 32.35 | 0.27 |
| Rec31 | WP_046103089.1 | 1.305 | 21.3 | 2.215 | 5.185 | 0.25 |
| Rec32 | WP_069027120.1 | 6.6 | 60.05 | 2.215 | 38.25 | 0.14 |
| Rec33 | WP_010671927.1 | 6.6 | 50.95 | 2.215 | 28.3 | 0.09 |
| Rec34 | WP_109653747.1 | 6.6 | 50.65 | 2.215 | 28.65 | 0.24 |
| Rec35 | WP_134161939.1 | 6.6 | 51.95 | 2.215 | 34.15 | 0.63 |
| Rec36 | WP_111534863.1 | 6.6 | 44.2 | 2.215 | 28.25 | 0.26 |
| Rec37 | WP_128085508.1 | 6.6 | 40 | 2.215 | 15.85 | 0.36 |
| Rec38 | WP_115764642.1 | 6.6 | 44.45 | 2.215 | 30.8 | 0.06 |
| Rec39 | WP_11H38305.1 | 6.6 | 42 | 2.215 | 14.845 | 0.33 |
| Rec82 | WP_056773790.1 | 5.03 | 59 | 3.47 | 5.625 | NT |
| Rec83 | WP_033768926.1 | 5.425 | 65.2 | 3.47 | 4.43 | NT |
| Rec142 | WP_048474244.1 | 4.4 | 20.25 | 0.9 | 0.325 | NT |
| Rec338 | PKP94160.1 | 12.9 | 39.1 | 1.345 | 25.05 | 0.09 |
| Rec349 | WP_047138903.1 | 2.655 | 3.105 | 1.815 | 1.17 | NT |
| Rec432 | WP_016115818.1 | 10.65 | 7.59 | 3.03 | 1.015 | NT |
| Rec476 | WP_037412868.1 | 10.255 | 9.875 | 3.995 | 3.94 | NT |
| Rec480 | WP_066605681.1 | 0.49 | 0.375 | 0.65 | 0.245 | NT |
| Rec483 | WP_040041154.1 | 3.015 | 3.625 | 1.45 | 1.33 | NT |
| Rec507 | WP_132978117.1 | 13.7 | 21.4 | 7.485 | 6.63 | NT |
| Rec521 | WP_111480623.1 | 7.83 | 57.3 | 8.22 | 7.42 | NT |
| Rec522 | WP_125440609.1 | 7.83 | 29.265 | 7.115 | 8.435 | NT |
| Rec523 | WP_065235645.1 | 9.44 | 46.5 | 4.3 | 2.39 | NT |
| Rec554 | WP_076797908.1 | 3.02 | 2.495 | 5.76 | 3.55 | NT |
| Rec555 | WP_097452609.1 | 1.23 | 47 | 9.2 | 10.525 | NT |
| Rec589 | WP_026351576.1 | NT | NT | 5.945 | 36.65 | 0.12 |
| Rec590 | WP_092743158.1 | NT | NT | 5.945 | 27.45 | NT |

### Example 16: Dual AAV Delivery of Tyrosine Recombinase and Template DNA to Mammalian Cells

This example describes the use of a tyrosine recombinase based Gene Writer system for the targeted integration of a template DNA into the human genome. More specifically, a recombinase, e.g., a tyrosine recombinase with an amino acid sequence from Table 1 or 2, and a template DNA comprising the associated recognition site, e.g., a sequence from Column 2 or 3 of Table 1, are co-delivered to HEK293T cells as separate AAV viral vectors to insert DNA precisely and efficiently in a mammalian cell genome comprising a cognate recognition site, e.g., a sequence from Column 3 of Table 1.

Two transgene configurations are assessed to determine the integration, stability, and expression using different AAV insert DNA formats: 1) template comprising a single recognition site that utilizes formation of double-stranded circularized DNA following AAV transduction in the cell nucleus; or 2) template comprising two same orientation recognition sites flanking the desired insert sequence, e.g., two copies of a recognition sequence from Column 2 or Column 3 of Table 1 in the same orientation, that can first be excised from the AAV genome by the recombinase for circularization followed by integration into the mammalian genome.

Adeno-associated viral vectors encoding a recombinase or the corresponding recognition site-containing insert DNA are generated based on the pAAV-CMV-EGFP-WPRE-pA viral backbone (Sirion Biotech), but with replacement of the CMV promoter with the EFla promoter. pAAV-Ef1a-Recombinase-WPRE-pA is generated using a human codon optimized recombinase (GenScript). pAAV-Stuffer insert DNA constructs additionally contain either a 500 bp stuffer sequence between the 5′ AAV2 ITR sequence and Ef1a promoter, or a 500 bp stuffer sequence proximal to the 5′ terminal AAV2 ITR sequence and a 500 bp stuffer sequence proximal to the 3′ AAV2 ITR sequence. The above listed AAV vectors are packaged into AAV2 serotype (Sirion Biotech) at a 10¹³ total vg scale.

HEK293T cells are seeded in a 48-well plate format at 40,000 cells/well. 24 h later, cells are transduced with either the AAV comprising the recombinase expression vector and the AAV comprising the insert DNA vector, or the AAV comprising the insert DNA vector alone (negative control). On days 3 and 7 post-transduction, genomic DNA is extracted to assess the efficiency of integration using dual AAV delivery of a tyrosine recombinase and an insert DNA vector comprising its recognition site. Integration events are assessed via ddPCR to quantify average integration events (copies/genome) across the cell population to estimate the fraction of cells successfully edited.

### Example 17: In Vitro Combination mRNA and AAV Delivery of a Gene Writing Polypeptide and Template DNA for Site-Specific Integration in Human Cells

This example describes use of a Gene Writer system for the site-specific insertion of exogenous DNA into the mammalian cell genome. More specifically, a recombinase, e.g., a tyrosine recombinase with an amino acid sequence from Table 1 or 2, and a template DNA comprising the associated recognition site, e.g., a sequence from Column 2 or 3 of Table 1, are introduced into HEK293T cells. In this example, the recombinase is delivered as mRNA encoding the recombinase, and the template DNA is delivered via AAV.

HEK293T cells are seeded in a 48-well plate format at 40,000 cells/well. 24 h later, cells are transduced with either mRNA encoding the recombinase polypeptide and an AAV comprising the insert DNA vector, or the AAV comprising the insert DNA vector alone (negative control). The timing of delivery is assessed by the following conditions: 1) mRNA delivery of recombinase and AAV delivery of template DNA on the same day, 2) mRNA delivery of recombinase 24 h prior to AAV delivery of template DNA, 3) AAV delivery of template DNA 24 h prior to mRNA delivery of recombinase. Genomic DNA is extracted three days post-transfection of mRNA and post-transduction of AAV to assess the efficiency of integration. Integration efficiency is assessed via ddPCR to quantify average integration events (copies/genome) across the cell population to estimate the fraction of cells successfully edited.

### Example 18: Ex Vivo Combination mRNA and AAV Delivery of a Gene Writing Polypeptide and Template DNA to HSCs for the Treatment of Beta-Thalassemia and Sickle Cell Disease

This example describes delivery of mRNA encoding a recombinase and AAV template DNA into C34+ cells (hematopoietic stem and progenitor cells) in order to write an actively expressed 7-globin gene cassette to treat genetic mutations that lead to beta-thalassemia and sickle cell disease.

In this example, AAV6 is used to deliver the template DNA. More specifically, the AAV6 template DNA includes, in order, 5′ ITR, a recombinase recognition site, e.g., a sequence from Column 2 or 3 of Table 1, a pol II promoter, e.g., the human β-globin promoter, a human fetal 7-globin coding sequence, a poly A tail and 3′ITR. Considering the maximum volume limit of electroporation reagents, recombinase mRNA and the AAV6 template are co-delivered into CD34 cells via different conditions, e.g.: 1) AAV6 template and recombinase mRNA are co-electroporated; 2) recombinase mRNA is electroporated 15 mins prior to AAV6 insert DNA transduction.

After electroporation/transduction, cells are incubated in CD34 maintenance media for 2 days. Then, ˜10% of the treated cells are harvested for genomic DNA isolation to determine integration efficiency. The rest of the cells are transferred to erythroid expansion and differentiation media. After ˜20 days differentiation, three assays are performed to determine the integration of 7-globin after erythroid differentiation: 1) a subset of cells is stained with NucRed (Thermo Fisher Scientific) to determine the enucleation rate; 2) a subset of the cells is stained with fluorescein isothiocyanate (FITC)-conjugated anti-γ-globin antibody (Santa Cruz) to determine the percentage of fetal hemoglobin positive cells; 3) a subset of the cells is harvested for HPLC to determine 7-globin chain expression.

### Example 19: Ex Vivo Delivery of a Gene Writer Polypeptide and Circular DNA Template for Generating CAR-T Cells

This example describes delivery of a Gene Writing system as a deoxyribonucleoprotein (DNP) to human primary T-cells ex vivo for the generation of CAR-T cells, e.g., CAR-T cells for treating B-cell lymphoma.

The Gene Writer polypeptide, e.g., recombinase, e.g., recombinase with a sequence from Table 1 or Table 2, is prepared and purified for use directly in its active protein form. For the template component, minicircle DNA plasmids that lack plasmid backbone and bacterial sequences are used in this example, e.g., prepared as according to a method of Chen et al. Mol Ther 8(3):495-500 (2003), wherein a recombination event is first used to excise these extraneous plasmid maintenance functions to minimize plasmid size and cellular response. The first recombination event may be performed by flanking the desired vector sequence with cognate recognition sites positioned in the same orientation, such that in vitro recombination with the cognate recombinase results in the formation of a minicircle template DNA comprising a single copy of the recombinase recognition site and desired sequence for integration, which is purified from the remaining plasmid vector. Template DNA minicircles comprise, in order, a recombinase recognition site, e.g., a sequence from Column 2 or 3 of Table 1, a pol II promoter, e.g., EF-1, a human codon optimized chimeric Antigen Receptor (including an extracellular ligand binding domain, a transmembrane domain, and intracellular signaling domains), e.g., the CD19-specific Hu19-CD828Z (Genbank MN698642; Brudno et al. Nat Med 26:270-280 (2020)) CAR molecule, and a poly A tail. The template DNA is first mixed with purified recombinase protein and incubated at room temperature for 15-30 mins to form DNP complexes. Then, the DNP complex is nucleofected into activated T cells. Integration by the Gene Writer system is assayed using ddPCR for molecular quantification, and CAR expression is measured by flow cytometry.

### Example 20: Production of mRNA Encoding a Gene Writer Polypeptide

This example describes the generation of a recombinase encoding mRNA by in vitro transcription from a DNA vector. The mRNA template plasmid includes the T7 promoter followed by a 5′UTR, the recombinase coding sequence, a 3′ UTR, and ˜100 nucleotide long poly(A) tail. The plasmid is linearized by enzymatic restriction resulting in blunt end or 5′ overhang downstream of poly(A) tail and used for in vitro transcription (IVT) using T7 polymerase (NEB). Following IVT, the RNA is treated with DNase I (NEB). After buffer exchange, enzymatic capping is performed using Vaccinia capping enzyme (NEB) and 2′-O-methyltransferase (NEB) in the presence of GTP and SAM (NEB). The capped RNA is purified and concentrated using silica columns (for example, Monarch® RNA Cleanup kit) and buffered by 2 mM sodium citrate pH 6.5.

### Example 21: Unidirectional Sequencing Assay for Determination of Integration Site

This example describes performance of unidirectional sequencing to determine the sequence of an unknown integration site with an unbiased profile of genome wide specificity. Integration experiments are performed as in previous examples by using a Gene Writing system comprising a recombinase and a template DNA for insertion. The recombinase and insert DNA plasmids are transfected into 293T cells. Genomic DNA is extracted at 72 hours post transfection and subjected to unidirectional sequencing according to the following method. First, a next generation library is created by fragmentation of the genomic DNA, end repair, and adaptor ligation. Next, fragmented genomic DNA harboring template DNA integration events is amplified by two-step nested PCR using forward primers binding to template specific sequence and reverse primers binding to sequencing adaptors. PCR products are visualized on a capillary gel electrophoresis instrument, purified, and quantified by Qubit (ThermoFisher). Final libraries are sequenced on a Miseq using 300 bp paired end reads (Illumina). Data analysis is performed by detecting the DNA flanking the insertion and mapping that sequence back to the human genome sequence, e.g., hg38.

### Example 22: Use of Dual AAV Vector for the Treatment of Cystic Fibrosis in CFTR Mouse Model

This example describes delivery of a Gene Writing system as a dual AAV vector system for the treatment of cystic fibrosis in a mouse model of disease. Cystic fibrosis is a lung disease that is caused by mutations in the CFTR gene, which can be treated by the insertion of the wild-type CFTR gene into the genome of lung cells, such as cells found in the respiratory bronchioles and columnar non-ciliated cells in the terminal bronchiole.

A Gene Writing polypeptide, e.g., comprising a sequence of Table 1 or Table 2, and a template DNA comprising a cognate recombinase recognition site, e.g., a sequence from Column 2 or 3 of Table 1, are packaged into AAV6 capsids with expression of the polypeptide driven by the CAG promoter, the combination of which has been shown to be effective for high level transduction and expression in murine respiratory epithelial cells according to the teachings of Halbert et al. Hum Gene Ther 18(4):344-354 (2007).

AAV preparations are co-delivered intranasally to CFTR gene knockout (Cftr^{tm1Unc}) mice (The Jackson Labs) using a modified intranasal administration, as described previously (Santry et al. BMC Biotechnol 17:43 (2017)). Briefly, AAVs are packaged, purified, and concentrated comprising either a recombinase expression cassette or template DNA, comprising the CFTR gene under the control of a pol II promoter, e.g., CAG promoter, and a cognate recombinase recognition site. In some embodiments, the CFTR expression cassette is flanked by the recombinase recognition sites. Prepared AAVs are each delivered at a dose ranging from 1×10¹⁰-1×10¹² vg/mouse using a modified intranasal administration to the CFTR knockout mouse. After one week, lung tissue is harvested and used for genomic extraction and tissue analysis. To measure integration efficiency, CFTR gene integration is quantified using ddPCR to determine the fraction of cells and target sites containing or lacking the insertion. To assay expression from successfully integrated CFTR, tissue is analyzed by immunohistochemistry to determine expression and pathology.

### Example 23: Method of Treating Ornithine Transcarbamylase Deficiency Through the Introduction of Transiently Expressed Integrase

This example describes the treatment of ornithine transcarbamylase (OTC) deficiency by the delivery and expression of an mRNA encoding a Gene Writer polypeptide, e.g., a recombinase sequence from Table 1 or Table 2, along with the delivery of an AAV providing the template DNA for integration. OTC deficiency is a rare genetic disorder that results in an accumulation of ammonia due to not having efficient breakdown of nitrogen. The accumulation of ammonia leads to hyperammonemia that can be debilitating and in severe cases lethal. The AAV template comprises a wild-type copy of the human OTC gene under the control of a pol II promoter, e.g., ApoE.hAAT, and a cognate recombinase recognition site, e.g., a sequence from Column 2 or 3 or Table 1. In some embodiments, the OTC expression cassette is flanked by the recombinase recognition sites.

In this example, LNP formulation of recombinase mRNA follows the formulation of LNP-INT-01 (methods taught by Finn et al. Cell Reports 22:2227-2235 (2018), incorporated herein by reference) and template DNA is formulated in AAV2/8 (methods taught by Ginn et al. JHEP Reports (2019), incorporated herein by reference). Briefly, OTC deficiency is restored by treating neonatal Spf^{ash} mice (The Jackson Lab) by injecting LNP formulations (1-3 mg/kg) containing the recombinase mRNA and AAV (1×10¹⁰-1×10¹² vg/mouse) containing the template DNA via the superficial temporal facial vein (Lampe et al. J Vis Exp 93:e52037 (2014)). The Spf^{ash} mouse has some residual mouse OTC activity which, in some embodiments, is silenced by the administration of an AAV that expresses an shRNA against mouse OTC as previously described (Cunningham et al. Mol Ther 19(5):854-859 (2011), the methods of which are incorporated herein by reference). OTC enzyme activity, ammonia levels, and orotic acid are measured as previously described (Cunningham et al. Mol Ther 19(5):854-859 (2011)). After 1 week, mouse livers are harvested and used for gDNA extraction and tissue analysis. The integration efficiency of hOTC is measured by ddPCR on extracted gDNA. Mouse liver tissue is analyzed by immunohistochemistry to confirm hOTC expression.

### Example 24: Use of a Gene Writing to Integrate a Large Payload into Human Cells

This example describes the recombinase-mediated integration of a large payload into human cells in vitro.

In this example, the Gene Writer polypeptide component comprises an mRNA encoding a recombinase, e.g., a recombinase sequence of Table 1 or Table 2, and a template DNA comprising: a cognate recombinase recognition site, e.g., a sequence of Column 2 or 3 of Table 1; a GFP expression cassette, e.g., a CMV promoter operably linked to EGFP; and stuffer sequence to bring the total plasmid size to approximately 20 kb.

Briefly, HEK293T cells are co-electroporated with the recombinase mRNA and large template DNA. After three days, integration efficiency and specificity are measured. In order to measure efficiency of integration, droplet digital PCR (ddPCR) is performed on genomic DNA e.g., as described by Lin et al. Hum Gene Ther Methods 27(5):197-208 (2016), using primer-probe sets that amplify across the junction of integration, e.g., with one primer annealing to the template DNA and the other to an appropriate flanking region of the genome, such that only integration events are quantified. Data are normalized to an internal reference gene, e.g., RPP30, and efficiency is expressed as the average integration events per genome across the population of cells. To measure specificity, integration events in genomic DNA are assessed by unidirectional sequencing to determine genome coordinates, as described in Example 21.

### Example 25: Use of a Gene Writing to Integrate a Bacterial Artificial Chromosome into Human Embryonic Stem Cells Ex Vivo

This example describes the recombinase-mediated integration of a bacterial artificial chromosome (BAC) into human embryonic stem cells (hESCs).

BAC vectors are capable of maintaining extremely large (>100 kb) DNA payloads, and thus can carry many genes or complex gene circuits that may be useful in cellular engineering. Though there has been demonstration of their integration into hESCs (Rostovskaya et al. Nucleic Acids Res 40(19):e150 (2012)), this was accomplished using transposons that lack sequence specificity in their integration patterns. This Example describes sequence-specific integration of large constructs.

In this example, a BAC engineered to carry the desired payload further comprises a recombinase recognition sequence, e.g., a sequence of Column 2 or 3 from Table 1, that enables recognition by the Gene Writer polypeptide, e.g., a recombinase, e.g., a recombinase with a sequence of Table 1 or Table 2. An approximately 150 kb BAC is introduced into hESCs by electroporation or lipofection as per the teachings of Rostovskaya et al. Nucleic Acids Res 40(19):e150 (2012). After three days, integration efficiency and specificity are measured. In order to measure efficiency of integration, droplet digital PCR (ddPCR) is performed on genomic DNA e.g., as described by Lin et al. Hum Gene Ther Methods 27(5):197-208 (2016), using primer-probe sets that amplify across the junction of integration, e.g., with one primer annealing to the template DNA and the other to an appropriate flanking region of the genome, such that only integration events are quantified. Data are normalized to an internal reference gene, e.g., RPP30, and efficiency is expressed as the average integration events per genome across the population of cells. To measure specificity, integration events in genomic DNA are assessed by unidirectional sequencing to determine genome coordinates, as described in Example 21.

1. (canceled)

2. A system for modifying DNA comprising:
a) a recombinase polypeptide comprising an amino acid sequence selected from SEQ ID NO: 1241, SEQ ID NO: 1249, or comprising an amino acid sequence of Table 1 or 2, or an amino acid sequence having at least 70% identity thereto, or a nucleic acid encoding the recombinase polypeptide; and
b) an insert DNA comprising:
(i) a human first parapalindromic sequence and a human second parapalindromic sequence of Table 1 that bind to the recombinase polypeptide of (a).

3. A eukaryotic cell comprising the recombinase polypeptide of claim 7, or a nucleic acid encoding the recombinase polypeptide.

4. A eukaryotic cell comprising:
(i) a DNA recognition sequence, said DNA recognition sequence comprising a first parapalindromic sequence and a second parapalindromic sequence,
wherein each parapalindromic sequence is about 10-30, 12-27, or 10-15 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, 4, 5, 6, 7, or 8 sequence alterations relative thereto,
wherein said DNA recognition sequence further comprises a core sequence of about 5-10 nucleotides, and wherein the core sequence is situated between the first and second parapalindromic sequences; and
(ii) a heterologous object sequence;
wherein:
(a) the DNA recognition sequence is located within 1, 2, 3, 4, 5, 6, 7, 8, 9, 10, 15, 20, 25, 30, 40, 50, 60, 70, 80, 90, or 100 nucleotides of the heterologous object sequence; and/or
(b) the DNA recognition sequence and the heterologous objet sequence are extrachromosomal.

5. A method of modifying the genome of a eukaryotic cell comprising contacting the cell with:
a) a recombinase polypeptide comprising an amino acid sequence selected from SEQ ID NO: 1241, SEQ ID NO: 1249, or comprising an amino acid sequence of Table 1 or 2, or a sequence having at least 70%, 75%, 80%, 85%, 90%, 95%, 96%, 97%, 98%, or 99% identity thereto, or a nucleic acid encoding the recombinase polypeptide; and
b) an insert DNA comprising:
(i) a DNA recognition sequence that binds to the recombinase polypeptide of (a), said DNA recognition sequence comprising a first parapalindromic sequence and a second parapalindromic sequence, wherein each parapalindromic sequence is about 10-30, 12-27, or 10-15 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, or 4 sequence alterations relative thereto,
wherein said DNA recognition sequence further comprises a core sequence of about 5-10 nucleotides, and wherein the core sequence is situated between the first and second parapalindromic sequences, and
(ii) a heterologous object sequence,
thereby modifying the genome of the eukaryotic cell.

6. A method of inserting a heterologous object sequence into the genome of a eukaryotic cell comprising contacting the cell with:
a) a recombinase polypeptide comprising an amino acid sequence selected from Rec27 SEQ ID NO: 1241, SEQ ID NO: 1249, or comprising an amino acid sequence of Table 1 or 2, or a sequence having at least 70%, 75%, 80%, 85%, 90%, 95%, 96%, 97%, 98%, or 99% identity thereto, or a nucleic acid encoding the polypeptide; and
b) an insert DNA comprising:
(i) a DNA recognition sequence that binds to the recombinase polypeptide of (a), said DNA recognition sequence comprising a first parapalindromic sequence and a second parapalindromic sequence, wherein each parapalindromic sequence is about 10-30, 12-27, or 10-15 nucleotides, and the first and second parapalindromic sequences together comprise the parapalindromic region of a nucleotide sequence of Table 1, and
wherein said DNA recognition sequence further comprises a core sequence of about 5-10 nucleotides, and wherein the core sequence is situated between the first and second parapalindromic sequences, and
(ii) a heterologous object sequence,
thereby inserting the heterologous object sequence into the genome of the eukaryotic cell.

7. An isolated recombinase polypeptide comprising an amino acid sequence selected from SEQ ID NO: 1249, or comprising an amino acid sequence of Table 1 or 2 other than SEQ ID NO: 1241, or a sequence having at least 70%, 75%, 80%, 85%, 90%, 95%, 96%, 97%, 98%, or 99% identity thereto.

8. An isolated nucleic acid encoding the recombinase polypeptide of claim 7.

9. An isolated nucleic acid comprising:
(i) a DNA recognition sequence, said DNA recognition sequence comprising a first parapalindromic sequence and a second parapalindromic sequence, wherein each parapalindromic sequence is about 10-30, 12-27, or 10-15 nucleotides, and the first and second parapalindromic sequences together comprise the parapalindromic region of a nucleotide sequence of Table 1, and
said DNA recognition sequence further comprises a core sequence of about 5-10 nucleotides, wherein the core sequence is situated between the first and second parapalindromic sequences, and
(ii) a heterologous object sequence.

10. A method of making a recombinase polypeptide, the method comprising:
a) providing a nucleic acid encoding a recombinase polypeptide according to claim 7, and
b) introducing the nucleic acid into a eukaryotic cell under conditions that allow for production of the recombinase polypeptide,
thereby making the recombinase polypeptide.

11. A method of making an insert DNA that comprises a DNA recognition sequence and a heterologous sequence, comprising:
a) providing a nucleic acid comprising:
(i) a DNA recognition sequence that binds to a recombinase polypeptide according to claim 7, said DNA recognition sequence comprising a first parapalindromic sequence and a second parapalindromic sequence, wherein each parapalindromic sequence is about 10-30, 12-27, or 10-15 nucleotides, and the first and second parapalindromic sequences together comprise the parapalindromic region of a nucleotide sequence of Table 1, and
said DNA recognition sequence further comprises a core sequence of about 5-10 nucleotides, wherein the core sequence is situated between the first and second parapalindromic sequences, and
(ii) a heterologous object sequence, and
b) introducing the nucleic acid into a eukaryotic cell under conditions that allow for replication of the nucleic acid,
thereby making the insert DNA.

12. An isolated eukaryotic cell comprising a heterologous object sequence stably integrated into its genome at a genomic location listed in column 2 or 3 of Table 1.

13. The system of claim 2, wherein:
the insert DNA is a double-stranded DNA; and/or
the insert DNA comprises:
a DNA recognition sequence that binds to the recombinase polypeptide of (a),
said DNA recognition sequence comprising the first parapalindromic sequence and the second parapalindromic sequence, wherein each parapalindromic sequence is about 10-30, 12-27, or 10-15 nucleotides, and the first and second parapalindromic sequences together comprise the parapalindromic region of a nucleotide sequence of Table 1, or a nucleotide sequence having at least 70%, 75%, 80%, 85%, 90%, 95%, 96%, 97%, 98%, or 99% identity thereto, or having no more than 1, 2, 3, 4, 5, 6, 7, 8 sequence alterations relative thereto, and
said DNA recognition sequence further comprising a core sequence of about 5-10 nucleotides, wherein the core sequence is situated between the first and second parapalindromic sequences.

14. The system of claim 2, wherein the insert DNA comprises a heterologous object sequence.

15. The system of claim 2, wherein the recombinase polypeptide is selected from a recombinase listed in Table 16, or an amino acid sequence having at least 70%, 75%, 80%, 85%, 90%, 95%, 96%, 97%, 98%, or 99% identity thereto.

16. The system of claim 2, wherein the recombinase polypeptide of (a) and the insert DNA of (b) are in separate containers or admixed.

17. The system of claim 2, wherein the recombinase polypeptide comprises at least one insertion, deletion, or substitution relative to the amino acid sequence of Table 1 or 2.

18. The system of claim 2, wherein the recombinase polypeptide comprises a truncation at the N-terminus, C-terminus, or both of the N- and C-termini relative to the amino acid sequence of Table 1 or 2.

19. The system of claim 2, wherein the recombinase polypeptide comprises a nuclear localization sequence.

20. The system of claim 14, which results in an insert frequency of the heterologous object sequence into the genome of at least about 0.1%.
